(19) United States
(12) Patent Application Publication
(10) Pub. No.: US 2005/0266432 A1
(43) Pub. Date: Dec. 1, 2005

(54) Haplotype markers for diagnosing susceptibility to immunological conditions

(75) Inventors: Arnold Oliphant, Poway, CA (US); Sarah Murray, Carlsbad, CA (US)

Correspondence Address: FOLEY & LARDNER, P.O. BOX 80278, SAN DIEGO, CA, 92138-0278, US

(73) Assignee: Illumina, Inc.

(21) Appl. No.: 11/069,908

(22) Filed: Feb. 28, 2005

Related U.S. Application Data
(60) Provisional application No. 60/547,823, filed on Feb. 26, 2004.

(51) Int. Cl. C12Q 1/68

(52) U.S. Cl. 435/6

(57) ABSTRACT

The invention provides a method of identifying a SNP haplotype that correlates with an HLA type, MHC allele or susceptibility to a particular disease or condition including the steps of (a) providing the identity of the nucleotide for each of a set of single nucleotide polymorphism (SNPs) in the major histocompatability complex (MHC) region in a population of individuals; (b) providing the HLA type, MHC allele, or susceptibility to the particular disease or condition for the individuals; and (c) identifying a SNP haplotype in the population that correlates with the HLA type, MHC allele or susceptibility to the particular disease or condition, wherein the SNP haplotype includes the SNPs in the MHC region. The invention further provides a method for predicting an HLA type, MHC allele or susceptibility to a particular disease or condition of an individual including the steps of (a) providing the identity of a plurality of SNPs in one or more nucleic acids from the individual that correlate with an HLA type, MHC allele or susceptibility to a particular disease or condition; and (b) predicting the HLA type, MHC genotype or susceptibility to a particular disease or condition for the individual based on a SNP haplotype comprising the plurality of SNPs. The invention also provides SNPs and oligonucleotide probes that are useful for identifying haplotypes associated with a particular disease or condition or with the presence of particular protein variants involved in immunological recognition. The SNPs and probes are also useful for predicting HLA-type, HLA genotype or susceptibility to a particular disease or condition.

### CROSS-REFERENCE TO RELATED APPLICATIONS

This application claims priority of U.S. Provisional Application Ser. No. 60/547,823, the entire contents of which are hereby incorporated by reference thereto.

### FIELD OF THE INVENTION

The present invention relates generally to the field of genetic analysis. In particular the method relates to determining HLA-type and to predicting susceptibility to immunological or inflammatory conditions.

### BACKGROUND OF THE INVENTION

The following description of the background of the invention is provided simply as an aid in understanding the invention and is not admitted to describe or constitute prior art to the invention.

The major histocompatability complex (MHC) is a large genomic region that contains a series of linked genes that have critical roles the presentation of antigens to T-cells and in the recognition and discrimination between “self” and “non-self” molecules by T-cells. In humans, the MHC spans approximately 4 Mb and is located on the short arm of chromosome 6. The most extensively studied genes in the human MHC are the genes encoding the human leukocyte antigen (HLA) proteins. These proteins anchor in cell membranes and present antigenic peptides to the T lymphocytes resulting in the initiation of specific immune responses. The HLA proteins are encoded by two classes of genes, HLA class I and HLA class II. Class I HLA genes include HLA-A, HLA-B and HLA-C, and class II HLA genes include HLA-DR, HLA-DQ, HLA-DQB1, and HLA-DP.

Allelic variations in MHC genes have been shown to have associative or causal relationships with relative risk or predisposition to hundreds of diseases, especially diseases with immunological or inflammatory origin. Accordingly the identification of MHC alleles, or HLA-type, in an individual can be a powerful tool for predicting the individual's susceptibility to certain diseases and can also be used in disease diagnosis. Examples of diseases that have been implicated with MHC allelic variation include inflammatory bowel disease (IBD), ulcerative colitis, Crohn's disease, rheumatoid arthritis, diabetes, type I diabetes mellitus (IDDM), myasthenia gravis, vitiligo, Graves' disease, Hashimoto's disease, Addison's disease, autoimmune gastritis and autoimmune hepatitis, rheumatoid disease, systemic lupus erythematosus (SLE), progressive systemic sclerosis and variants, polymyositis, dermatomyositis, pernicious anemia including some of autoimmune gastritis, primary biliary cirrhosis, autoimmune thrombocytopenia, Sjogren's syndrome, multiple sclerosis (MS), Reiter's syndrome and psoriasis.

HLA molecules play a central role in the recognition and subsequent rejection of transplanted tissues. As such, HLA-typing is used to identify favorable donor-recipient pairs for grafting bone marrow and for solid organ transplantation. The numerous alleles of HLA genes in the population also make HLA typing useful for paternity testing.

Currently, HLA typing is performed using serological or molecular methods which are laborious and/or expensive. Serological-based HLA typing has traditionally been the predominant method for donor-recipient tissue matching for tissue transplantation. Serological detection of HLA class I and II antigens has been accomplished using a complement mediated lymphocytotoxicity test with purified T or B lymphocytes. This procedure is predominantly used for matching HLA-A and -B loci, and currently is often used only as an initial step in HLA-typing.

Molecular-based tissue typing can often be more accurate than serologic testing. Low resolution molecular methods such as SSOP (sequence specific oligonucleotide probes) methods, in which PCR products are tested against a series of oligonucleotide probes, can be used to identify HLA antigens, and currently these methods are the most common methods used for Class II-HLA typing. High resolution techniques such as SSP (sequence specific primer) methods which utilize allele specific primers for PCR amplification can identify specific MHC alleles, however these methods can be extremely laborious and expensive.

### SUMMARY OF THE INVENTION

The present invention provides methods and compositions for identifying genetic markers associated with HLA alleles and alleles of other loci in the MHC region. The methods are generally useful for identifying genetic markers for alleles associated with the expression of proteins involved in immunological recognition. Such methods are useful for predicting the presence of alleles associated with an immunological or inflammatory condition. In particular embodiments, the present invention pertains to the identification of SNPs in the major histocompatibility complex (MHC) that correlate with HLA alleles that are transplantation or disease related. The invention also pertains to methods for identifying SNPs in the major histocompatibility complex (MHC) that correlate to alleles that are causal to or associated with an immunological or inflammatory condition.

The invention provides a method of identifying a SNP haplotype that correlates with an HLA type. The method can include the steps of (a) providing the identity of the nucleotide for each of a set of single nucleotide polymorphisms (SNPs) in the major histocompatability complex (MHC) region in a population of individuals; (b) providing the HLA type for the individuals; and (c) identifying a SNP haplotype in the population that correlates with the HLA type, wherein the SNP haplotype includes the SNPs in the MHC region.

The invention further provides a method for predicting the HLA type of an individual. The method can include the steps of (a) providing the identity of a plurality of SNPs in one or more nucleic acids from the individual that correlate with an HLA type; and (b) predicting the HLA type of the individual based on a SNP haplotype comprising the plurality of SNPs.

In related aspects, the invention provides a method of identifying a SNP haplotype that predicts an HLA type. The method can include the following steps (a) determining the identity of the nucleotide for each of a set of single nucleotide polymorphisms (SNPs) in the major histocompatability complex (MHC) region in a population of individuals; (b) determining the HLA type for said individuals; and (c) identifying a SNP haplotype by correlating SNP genotypes with HLA type in said population, wherein an SNP haplotype that predicts HLA type is identified. In other related aspects the invention provides a method for identifying genetic markers for predicting HLA alleles. The method can involve (a) determining the identity of the nucleotide for each of a set of single nucleotide polymorphism (SNPs) in the major histocompatability complex (MHC)in a population of individuals typed for transplantation related or disease related HLA alleles; and (b) identifying one or more SNP genotypes in the population that correlate with particular HLA transplantation related or disease related alleles. In another aspect the invention provides a method for determining the HLA type of a patient. The method involves (a) determining in nucleic acids from the patient the genotype for each of a panel of SNPs that correlate with transplantation related or disease related HLA alleles; and (b) determining the HLA alleles that correlate with the identified SNP genotypes wherein the SNPs that correlate with transplantation related or disease related HLA alleles are identified using the method described above.

Also provided is a method of identifying a SNP haplotype that correlates with an allelic variant of an MHC gene. The method can include the steps of (a) providing the identity of the nucleotide for each of a set of single nucleotide polymorphisms (SNPs) in the major histocompatability complex (MHC) region in a population of individuals, wherein the set of SNPs includes a SNP that is outside of the translated region(s) of an MHC gene; (b) providing the presence or absence of the allelic variant of an MHC gene in the individuals; and (c) identifying a SNP haplotype in the population that correlates with the allelic variant of an MHC gene, wherein the SNP haplotype includes the set of SNPs in the MHC region.

The invention also provides a method for determining the presence or absence of an allelic variant of an MHC gene in an individual. The method can include the steps of (a) providing the identity of a plurality of SNPs in one or more nucleic acids from the individual that correlate with MHC genotype, wherein the set of SNPs includes a SNP that is outside of the translated region(s) of the MHC gene; and (b) determining the MHC genotype of the individual based on a SNP haplotype including the plurality of SNPs.

The invention further provides a method for identifying a SNP haplotype that correlates with susceptibility to a disease. The method can include the steps of (a) providing the identity of the nucleotide for each of a set of single nucleotide polymorphism (SNPs) in the major histocompatability complex (MHC) region in a population of individuals; (b) providing susceptibility to the disease for the individuals; and (c) identifying a SNP haplotype in the population that correlates with the susceptibility to the disease, wherein the SNP haplotype includes the SNPs in the MHC region.

Also provided is a method for determining the susceptibility of an individual to a disease. The method can include the steps of (a) providing the identity of a plurality of SNPs in one or more nucleic acids from the individual that correlate with susceptibility to the disease; and (b) determining the susceptibility of the individual to the disease based on a SNP haplotype including the plurality of SNPs.

In yet another aspect, the invention provides a method for predicting whether a patient is a carrier of an allele associated with an immunological or inflammatory condition, said method comprising (a) evaluating linkage disequilibrium (LD) for at least one SNP in a region of the major histocompatability complex and at least one HLA allele associated with or causal to a immunological or inflammatory condition in a population of individuals, wherein said population comprises individuals that are carriers of said HLA alleles and individuals that are not carriers of said HLA alleles; (b) determining the LD for the at least one SNP and the at least one HLA allele in said patient to and correlating said LD to the results of step (a) to predict whether said patient carries one or more of said HLA alleles.

In related aspects the invention provides a method for identifying genetic markers for predicting the presence of alleles associated with an immunological or inflammatory condition. The method can include (a) determining the identity of the nucleotide for each of a set of single nucleotide polymorphism (SNPs) in the major histocompatability complex (MHC) in a population of individuals, wherein said population comprises individuals known to carry an allele causal to or associated with an immunological or inflammatory condition and individuals wherein said allele is absent; (b) identifying one or more SNP genotypes in the population that correlate with the presence or absence of said allele in said individuals to identify a one or more SNP genotypes that correlate with said allele causal to or associated with an immunological or inflammatory condition. In certain aspects the invention provides a method for predicting the presence of alleles associated with an immunological or inflammatory condition in a patient by (a) determining in nucleic acids from the patient the genotype for each of a panel of SNPs that correlate with alleles associated with an immunological or inflammatory condition; and (b) determining the alleles alleles associated with an immunological or inflammatory condition that correlate with the identified SNP genotypes.

The invention also provides a haplotype map; that has one or more SNPs wherein said SNPs are in the MHC region between the RFP gene and the MLN gene of human chromosome 6. The SNPs can span the MHC region at an average density of at least SNPs per 15 kb of genomic DNA, and wherein the SNPs include genotypes that together can correlate with a specific HLA-type. It will be understood that the density of SNPs can be greater in or around a particular translated region of an MHC gene.

In another aspect, the invention provides a SNP haplotype for predicting HLA type, HLA genotype, or susceptibility of an individual to a disease or condition such as an immunological or inflammatory condition. The SNP haplotype can include at least 4 SNPs in the MHC region In yet another aspect the invention provides a set of oligonucleotide probes for detecting one or more SNPs of a SNP haplotype. The set of SNPs can include at least 4 SNPs in a the MHC region.

The invention also provides a set of SNPs for HLA-typing or for predicting susceptibility of an individual to an immunological or inflammatory condition. The set of SNPs of the invention may comprise at least about 4, or at least about 10, or at least about 100, or at least about 1,000, or more than 1, 000 SNPs in a region of the major histocompatability complex (MHC). In some preferred embodiments the SNPs of the set of SNPs of the invention cover the MHC region at a an average density of at least at least 1 SNP locus per 15 kb of genomic DNA or 1 locus/10 kb, 1 locus/8 kb, 1 locus/5 kb, 1 locus/2 kb, 1 locus/1.5 kb, 1 locus/1.2 kb, 1 locus/1 kb, 1 locus/ 0.5 kb or higher. In certain aspects a set of SNPs of the present invention comprise at least about 10, at least about 50, at least about 100, at least about 500 or more than 500 SNPs of the SNPs listed on Table 1.

In other aspects the invention provides a set of oligonucleotides for detecting a set of SNPs. The oligonucleotides of the present invention may comprise one or more oligonucleotides selected from the group consisting of the oligonucleotides shown on Table 2. In certain embodiments the set of oligonucleotides of the invention may comprise at least about 10, at least about 50, at least about 100, at least about 500, at least about 1000, at least about 2000 or more than 2000 of the oligonucleotides shown on Table 2.

As used herein, the terms “single nucleotide polymorphism” or “SNP” are intended to mean a location of genomic DNA sequence variation in a population of individuals, wherein the location is occupied by one of at least two different nucleotides. The term is used herein consistent with its meaning in the related art. Typically, a SNP is binary having two variants in a population such as G/A, G/T, G/C, A/T, A/C or C/T. However, a SNP can have more than two variants such as three or four variants. The location of a SNP in a genome sequence can be identified, for example, according to a sequence, flanking one or both sides of the SNP, that is the same or similar for individuals that have different SNPs. The flanking sequence can be any length sufficient to identify the SNP including, for example, at least about 10, 25, 50, 75, 100, 125, 150 or 200 nucleotides. A SNP can also be located according to coordinates in a particular genome sequence such as a genome build available from the National Center for Biotechnology Information (NCBI) or other source. A SNP can be located in a coding or non-coding region of a gene.

The term “haplotype” as used herein, refers to a set of alleles for two or more linked loci on a chromosome that tend to be inherited together in an individual or population of individuals. The loci can be, for example, SNPs or other genetic markers, such as RFLPs, that are commonly inherited. SNPs that are more uniquely associated with a particular haplotype are known in the art as “tag SNPs.”

As used herein, the term “MHC haplotype” refers to a set of alleles for two or more loci in the MHC region that tend to be inherited together. An “HLA haplotype” is understood to include HLA alleles and, because HLA alleles are found in the MHC region, is a species of an MHC haplotype. A “SNP haplotype” as used herein, refers to a set of SNPs for two or more linked loci on a chromosome that tend to be inherited together.

As used herein, the term “disease related MHC alleles” refers to genetic alleles in the MHC region that are associated with or causal to particular diseases or conditions. As set forth in further detail below, MHC alleles can be associated with an immunological condition, autoimmune disease, or schizophrenia among other conditions.

The term “allelic variant” as used herein, refers to a specific nucleotide or sequence variation at a genetic locus. Examples of such nucleotide variations can include mutations, deletions, duplications or SNPs. In certain embodiments an allelic variant of the invention may be in the promoter region or in the coding region of a gene. In some embodiments, an allelic variant of the invention are in a gene in the MHC region. Accordingly, a genetic locus useful in the invention can be within, for example, a gene, exon, intron, transcribed region, translated region, untranslated region or region of a genome having one or more other annotations or functions. In certain preferred embodiments, an allelic variant of the invention may be associated with a specific phenotype. In preferred embodiments an allelic variant of an MHC gene of the invention may be associated with or causal to a specific phenotype, for example, a histocompatibility phenotype or a phenotype associated with susceptibility to disease. An allelic variant of an MHC gene may be a disease related MHC allele. In some embodiments an allelic variant of an MHC gene may be an HLA haplotype. In other embodiments an allelic variant of an MHC gene may be a “histocompatibility related allele”. As used herein, the term “histocompatibility related allele” refers to alleles that are associated with or causal to recognition of immunologic similarity (or identity). Such recognition mediates, for example, heterograft rejection, successful homograft transplantation, and blood transfusion compatibility. HLA alleles or MHC alleles can contribute to the histocompatibility between individuals and may be used to determine favorable donor-recipient pairs for tissue transplants or blood transfusions.

The term “HLA-type” refers to the complement of HLA antigens present on the cells of an individual. An individual's HLA-type may be used to predict favorable donor-recipient pairs for tissue transplant or blood transfusion or may be used as an indicator of the individual's susceptibility to certain diseases or conditions. In particular, an individual's HLA serotype can be used to predict compatibility between a blood transfusion donor and recipient. An HLA-type can be determined according to the proteins expressed from particular alleles of genes in the MHC region; for example an HLA-type can refer to specific HLA class I proteins or HLA class II proteins. In certain embodiments, genes that may be represented in an HLA-type include one or more genes selected from the group consisting of HLA-A, HLA-B, HLA-Cw, HLA-DR, HLA-DQ and HLA-DP. Terminology for specific HLA-types is usually expressed in accordance with reports released by the World Heath Organization Committee on Nomenclature.

The term “HLA gene” as used herein, refers to a genomic nucleotide sequence that expresses an HLA class I or HLA class II proteins. Class I HLA genes include HLA-A, HLA-B and HLA-C, and class II HLA genes include HLA-DR, HLA-DQ, HLA-DQB1, and HLA-DP. The genes include a coding region which is a portion of the genomic sequence that is transcribed into mRNA and translated into a protein product. The genes further include portions of the genomic sequence that regulate expression of particular protein products.

The term “linkage disequilibrium” as used herein, refers to a pattern of inheritance wherein alleles at genetically linked loci are transmitted together in individuals more frequently than would be expected by chance. When the observed frequencies of haplotypes in a population does not agree with haplotype frequencies predicted by multiplying together the frequency of individual genetic markers in each haplotype then linkage disequilibrium is said to occur. Also known in the art as “allelic association,” linkage disequilibrium is generally a function of the distance on a chromosome between loci. For example, when alleles at two distinctive loci occur in gametes more frequently than expected given the known allele frequencies and recombination fraction between the two loci, the alleles are said to be in linkage disequilibrium.

The term “haplotype map” as used herein, refers to a collection of genetic loci that are useful for describing common patterns of genetic variation. A haplotype map usually comprises specific genetic loci that are representative of certain haplotypes. Haplotype maps can show the extent of linkage disequilibrium in a specific region of the genome. A haplotype map can be used to identify chromosome regions that have SNPs that are strongly associated and to identify chromosomal regions where associations among SNPs are weak. Thus a haplotype map can be useful for identifying specific SNPs in regions of a chromosome with strong SNP associations that can be used to predict certain haplotypes.

### DETAILED DESCRIPTION OF THE INVENTION

The present invention relates to methods of identifying a haplotype of SNPs or other genetic markers that correlates with proteins involved in immunological recognition such as proteins encoded by the MHC region including, but not limited to, HLA proteins. Different variants of proteins involved in immunological recognition, such as HLA proteins, can be associated with different genetic markers such as SNPs. Thus, in particular embodiments, a method of the invention can be used to predict the HLA type of an individual based on correlation to a haplotype. A correlation identified in a method of the invention can allow unique identification of a particular HLA type such that it can be distinguished from other HLA types. Thus, a haplotype identified in a method of the invention provides a surrogate marker for HLA type allowing a genetic analysis to be used to augment or replace traditional HLA typing methods. Similarly, the methods can be used to correlate a haplotype with other phenotypes and a genetic analysis can be used to augment or replace traditional methods of determining the phenotype. Exemplary phenotypes include, but are not limited to those set forth in further detail below.

The invention further provides a method of identifying a haplotype that correlates with an HLA genotype. A haplotype identified in a method of the invention need not be limited to alleles for loci that are located in the coding region of an MHC region. Thus, an advantage of the invention is that one or more alleles that are outside of the coding region of an MHC gene can be used to predict an HLA genotype. Because an HLA genotype can be indicative of HLA type or other phenotypes such as predisposition to disease or tissue compatibility, the methods provide for increased accuracy in diagnostic or prognostic applications. Furthermore, the invention can be used to identify a haplotype that correlates with predisposition to disease or tissue compatibility whether or not an associated HLA genotype is known.

A method for correlating a haplotype with a particular genotype or phenotype can be carried out using a population of individuals. Sequence information for one or more individuals can be provided in a method of the invention by carrying out known sequencing methods using genetic material obtained from the individual. Alternatively or additionally, sequence information can be provided in the form of an information storage medium such as a computer readable memory, hard copy (such as paper and other written media), a computer network or database. Similarly, phenotype information for one or more individuals can be provided in the form of an information storage medium or by carrying out known methods of determining phenotype by evaluating the individual. For example, HLA type can be determined using methods described in the background section above. For each individual the genotype or phenotype can be determined using a diagnostic or research method.

A haplotype that has been correlated with a particular genotype or phenotype is useful for predicting whether an individual, who was not necessarily included in the population for which the correlation was identified, has the phenotype or genotype. Thus, the invention provides a method of predicting a genotype or phenotype for an individual by determining the presence of a particular haplotype in the genome of the individual. Exemplary genotypes that can be predicted in an individual include, without limitation, MHC genotype or HLA genotype and exemplary phenotypes include, but are not limited to, histocompatibility, susceptibility to a particular disease or condition, the presence of particular MHC proteins or the presence of particular HLA proteins.

The present invention also relates to methods for identifying genetic markers that correlate with the presence of alleles associated with immunological or inflammatory conditions. The present invention provides a haplotype map for predicting whether a patient has an allele associated with immunological or inflammatory conditions. In certain aspects the invention provides a set of SNPs for predicting susceptibility of an individual to immunological or inflammatory conditions. Exemplary SNPs are listed in Table 1. The invention also provides a set of oligonucleotide probes. The oligonucleotide probes are particularly useful for identifying HLA alleles and HLA-haplotypes. Exemplary probes are listed in Table 2.

The invention provides a method of diagnosing or predicting susceptibility to an immunological condition or inflammatory condition by determining the presence or absence of a haplotype, wherein the presence of the haplotype is diagnostic or predictive of the immunological condition or inflammatory condition. Typically, the haplotype will have at least one SNP. The SNP can be located in the MHC region. The SNP can be located at a genomic location outside of an HLA gene.

The invention further provides a method of determining HLA-type by determining the presence or absence of a haplotype, wherein the presence of the haplotype is associated with the HLA-type. Typically, the haplotype will have at least one SNP. The SNP can be located in the MHC region. The SNP can be located at a genomic location outside of an HLA gene.

Also provided is a method of identifying a diagnostic haplotype for an immunological condition or inflammatory condition. The method includes the steps of (a) evaluating linkage disequilibrium for at least one SNP and at least one HLA allele in a population of individuals, wherein the HLA allele is associated with an immunological condition or inflammatory condition; and (b) determining that the at least one SNP is in linkage disequilibrium with the at least one HLA allele, thereby identifying a diagnostic haplotype for the immunological condition or inflammatory condition, wherein the diagnostic haplotype has the at least one SNP and the at least one HLA allele. The SNP can be located in the MHC region. The SNP can be located in a gene other than an HLA gene.

An HLA allele useful in a method of the invention can be an allele of a class I gene including, for example, HLA-A, HLA-B or HLA-C. An HLA allele useful in a method of the invention can be an allele of a class II gene including, for example, HLA-DRB 1, HLA-DQA1, HLA-DQB1, HLA-DPA1 or HLA-DPB1.

A haplotype useful in a method of the invention can include one or more of the single nucleotide polymorphisms (SNPs) listed in Table 1. Table 1 shows a list of single nucleotide polymorphism (SNP) loci that can be used in various combinations to diagnose or predict susceptibility to an immunological condition. All loci were identified on Homo sapiens chromosome 6 as identified in genome build version 33 of a diploid set of chromosomes (available from the NCBI, the website of which is located on the worldwide web at ncbi.nlm.nih.gov) The first column indicates the name of the SNP from dbSNP, the version of dbSNP being listed in column 3. The coordinates locating each SNP on chromosome 6 (build 33) are listed in column 2.

A candidate list of SNPs for use in the methods of the invention may be selected by mining known public databases (dbSNP;http://www.ncbi.nlm.nih.gov/projects/SNP/) for SNPs in the MHC region. In certain embodiments the SNPs are selected from the MHC region defined by the region flanked by genes Ret Finger Protein (RFP) to Motilin (MLN) designated by the NCBI genome assembly build 33 coordinates from 28,933,330 to 33,773,208. A region that is useful in the invention can include sequences flanking the MHC region, if desired, including, for example, the region that spans 10 kb past the RFP and MLN genes which is from the build 33 coordinates of 28,923,330 to 33,783,208. Those skilled in the art will recognize that smaller regions within these coordinates or larger regions including these coordinates or portions of both can be used in the invention in accordance with the methods exemplified for the region flanked by the RFP and MLN genes. In certain preferred embodiments of the invention, a subset of all SNPs in a particular region, such as the MHC region may be identified and selected for the method based on 2 different criteria; (1) SNPs within 10 kb of coding sequence to all genes in the MHC region may be chosen with higher density of SNPs chosen around the HLA genes, and (2) other densely spaced SNPs (1 SNP every 1-2 kb) can be chosen for uniform spacing throughout the MHC region. In preferred embodiments, these two criteria are used to choose SNPs as candidates (or a “database” of candidate SNPs) to determine haplotypes in the MHC region.

A haplotype useful in the invention can include at least 1, 2, 3, 4, 5, 10, 20, 30, 40, 50, 75, 100, 200, 400, 600, 800, 1000, 1500, 2000, or more SNPs including, for example, one or more of those listed in Table 1 or otherwise disclosed herein. A haplotype can further include one or more other genetic loci such as a deletion, insertion, mutation, rearrangement or the like. A particularly useful genetic locus that can be included in a haplotype is a causal allele. A causal allele is a mutation or polymorphism in a gene that renders an individual susceptible to a particular condition. It will be understood, however, that a haplotype useful for diagnosing or predicting susceptibility to a particular condition need not include a causal variant, so long as the haplotype is associated with the condition or a symptom of the condition.

In particular embodiments, genetic loci of a haplotype, such as SNPs, can be distributed at an average density of, for example, at least about 1 locus/15 kb, 1 locus/10 kb, 1 locus/8 kb, 1 locus/5 kb, 1 locus/2 kb, 1 locus/1.5 kb, 1 locus/1.2 kb, 1 locus/1 kb, 1 locus/0.5 kb or higher.

This disclosure describes methods for building a high density single nucleotide polymorphism (SNP) haplotype map in the major histocompatibility complex (MHC) region. Accordingly, this disclosure provides a haplotype map having at least one SNP in the MHC region. The haplotype map can include a SNP that occurs in a known human leukocyte-antigen (HLA) gene. Alternatively or additionally, the haplotype map can include a SNP that occurs outside of a sequence for a known HLA gene. For example, a SNP useful for a haplotype map of the invention can occur in a sequence that flanks an HLA gene. An advantage of the SNP haplotype map is that it includes “surrogate” markers to known HLA alleles. A haplotype disclosed herein or identified by a method disclosed herein can be used for diagnosis of immune conditions or inflammatory conditions. A haplotype can also be used to identify one or more causative alleles for a disease or condition, thereby aiding in identification of a useful therapy to treat the disease or condition.

In some embodiments of the invention, selected SNPs may be genotyped in various control populations to determine haplotype structure. Haplotype structures can be determined using various publicly available analysis packages such as Haploview (Bioinformatics. Jan. 15, 2005;21(2):263-5) from either parent-offspring trios or unrelated individuals. Haplotype blocks can be defined using the methods known in the art, such as those described in Gabriel et al. (Science. Jun. 21, 2002;296(5576):2225-9). The various control populations can vary by ethnicity as it is known that haplotype structure and diversity varies by ethnic group. The number of individuals included in a population for identifying a genetic correlation can be chosen according to statistical and genetic methods known in the art. The number can be, for example, at least about 2, 10, 25, 50, 100, 200, 300, 400, 500, 1000 or more individuals.

In the invention HLA typing may be performed by serological or DNA based typing methods. In preferred embodiments, four-digit HLA types for HLA genes such as HLA-A, HLA-B, HLA-C, HLA-DRB1, DMB1, DQA1, DQB1, and DPB1 are determined by hybridization-based molecular typing methods. Single-block or multi-block SNP haplotypes can be correlated with HLA genotypes. One method for correlating HLA genotypes with SNP haplotypes is the “Leave-one-out” cross-validation as described by Walsh et al. (Am J Hum Genet. September 2003;73(3):580-90). Using this method, one can assess the predictability of an HLA allele with a SNP haplotype. Conversely, one can assess the predictability of a SNP haplotype with an HLA allele.

The methods of the present invention are useful for diagnosing or predicting susceptibility to a variety of immunological conditions including, for example, an autoimmune disease. As used herein, the term “autoimmune disease” means a disease resulting from an immune response against a self tissue or tissue component, including both self antibody responses and cell-mediated responses. The term autoimmune disease, as used herein, encompasses organ-specific autoimmune diseases, in which an autoimmune response is directed against a single tissue, such as Crohn's disease and ulcerative colitis, Type 1 diabetes mellitus (IDDM), myasthenia gravis, vitiligo, Graves' disease, Hashimoto's disease, Addison's disease and autoimmune gastritis and autoimmune hepatitis. The term autoimmune disease also encompasses non-organ specific autoimmune diseases, in which an autoimmune response is directed against a component present in several or many organs throughout the body. Such autoimmune diseases include, for example, rheumatoid disease, systemic lupus erythematosus (SLE), progressive systemic sclerosis and variants, polymyositis and dermatomyositis. Additional autoimmune diseases include pernicious anemia including some of autoimmune gastritis, primary biliary cirrhosis, autoimmune thrombocytopenia, Sjogren's syndrome, multiple sclerosis (MS) Reiter's disease and psoriasis. One skilled in the art understands that the methods of the invention can be applied to these or other autoimmune diseases, as desired. Exemplary autoimmune diseases include, but are not limited to, inflammatory bowel disease (IBD) Crohn's disease, rheumatoid arthritis or diabetes.

In certain embodiments SNP haplotypes determined to predict HLA genotypes can be used in association studies of various disease phenotypes such as inflammatory or auto-immune disorders. Association studies include such methods as case/control studies and family-based association studies including the transmission disequilibrium test (TDT) or pedigree disequilibrium test (PDT). Other applications of this method include the use of SNP haplotypes as a diagnostic marker for known HLA alleles (genotypes).

A haplotype can be diagnostic or predictive of a particular stage or symptom of an immunological condition. In this regard, an autoimmune reaction is directed against the brain in multiple sclerosis and the gut in Crohn's disease. In other autoimmune diseases such as systemic lupus erythematosus, affected tissues and organs may vary among individuals with the same disease. Ultimately, damage to certain tissues by the immune system may be permanent, as with destruction of insulin-producing cells of the pancreas in Type 1 diabetes mellitus. Accordingly, a haplotype can be diagnostic or predictive of tissue damage.

In a number of autoimmune diseases including, for example, Graves, disease (GD), Rheumatoid Arthritis (RA), myasthenia gravis, insulin-resistant diabetes (Type I), antibodies to cell membrane receptors lead to anti-receptor hypersensitivity reactions that alter cellular function as a result of the binding of antibody to membrane receptors, which can have a stimulatory or a blocking effect. Similarly, in some diabetic patients with extreme insulin resistance, antibodies to insulin receptors have been shown that prevent the binding of insulin to its receptor. An important aspect of Graves' disease concerns the presence in most patients of detectable IgGs directed against the thyroid-stimulating hormone receptor (TSHR). These antibodies can possess stimulatory or blocking activities, and depending on which type predominates, result in either thyroid gland over- or under-activity. Accordingly, a haplotype can be diagnostic or predictive of anti-receptor hypersensitivity reactions.

The methods of the invention can also be used to diagnose or predict susceptibility to an inflammatory condition. Exemplary conditions include, without limitation, inflammatory lung diseases such as adult respiratory distress syndrome, asthma, emphysema, chronic bronchitis, cystic fibrosis, or interstitial lung disease such as interstitial pneumonitis, idiopathic fibrosis and interstitial fibrosis.

The methods of the present invention can also be used for diagnosing or predicting susceptibility to schizophrenia.

A variety of symptoms that are indicative of the above-described conditions or diseases are well known in the art as described, for example, in The Merck Manual of Diagnosis and Therapy 16^{th} Ed., Edited by Berkow, Published by Merck and Co., Inc., Rahway N.J. (1992). A haplotype disclosed herein or identified by a method disclosed herein can be diagnostic or predictive of one or more of such symptoms.

A method of the invention can include a step of obtaining material containing one or more target nucleic acids having a desired SNP such as one or more of the SNP loci set forth in Table 1. As used herein, the term “material” means any biological matter from which a target nucleic acid can be prepared or isolated. For example, the term material encompasses whole blood, plasma, saliva or other bodily fluid or tissue that contains nucleic acid. A preferred material is whole blood, which can be obtained readily by non-invasive means and used to prepare genomic DNA for detection using a method described herein. Other materials are set forth below in further detail. As used herein, the term “nucleic acid” means a polynucleotide such as deoxyribonucleic acid (DNA) or ribonucleic acid (RNA). The term can include either single-stranded or double-stranded nucleic acid. Total genomic DNA, fragments thereof or amplified representations thereof are particularly useful nucleic acids with which to practice a method of the invention.

Accordingly, the term “amplified representative” is intended to mean a nucleic acid copy in which the proportion of each sequence in the copy relative to all other sequences in the copy is substantially the same as the proportions in the nucleic acid template. When used in reference to a population of genome fragments, for example, the term is intended to mean a population of genome fragments in which the proportion of each genome fragment to all other genome fragments in the population is substantially the same as the proportion of its sequence to the other genome fragment sequences in the genome. Substantial similarity between the proportion of sequences in an amplified representation and a template genomic DNA means that at least 60% of the loci in the representation are no more than 5 fold over-represented or under-represented. In such representations at least 70%, 80%, 90%, 95% or 99% of the loci can be, for example, no more than 5, 4, 3 or 2 fold over-represented or under-represented. A nucleic acid included in the term can be DNA, RNA or an analog thereof. The number of copies of each nucleic acid sequence in an amplified representative population can be, for example, at least. 2, 5, 10, 25, 50, 100, 1000, 1×10⁴, 1×10⁵, 1×10⁶, 1×10⁷, 1×10⁸ or 1×10¹⁰ fold more than the template or more.

A target nucleic acid such as genomic DNA can be isolated from material including, for example, one or more cells, bodily fluids or tissues. Known methods can be used to obtain a bodily fluid such as blood, sweat, tears, lymph, urine, saliva, semen, cerebrospinal fluid, feces or amniotic fluid. Similarly known biopsy methods can be used to obtain cells or tissues such as buccal swab, mouthwash, surgical removal, biopsy aspiration or the like. A target nucleic acid having one or more SNP can also be obtained from one or more cell or tissue in primary culture, in a propagated cell line, a fixed archival sample, forensic sample or archeological sample.

Exemplary cell types from which a target nucleic acid can be obtained in a method of the invention include, without limitation, a blood cell such as a B lymphocyte, T lymphocyte, leucocyte, erythrocyte, macrophage, or neutrophil; a muscle cell such as a skeletal cell, smooth muscle cell or cardiac muscle cell; germ cell such as a sperm or egg; epithelial cell; connective tissue cell such as an adipocyte, fibroblast or osteoblast; neuron; astrocyte; stromal cell; kidney cell; pancreatic cell; liver cell; or keratinocyte. A cell from which target nucleic acid is obtained can be at a particular developmental level including, for example, a hematopoietic stem cell or a cell that arises from a hematopoietic stem cell such as a red blood cell, B lymphocyte, T lymphocyte, natural killer cell, neutrophil, basophil, eosinophil, monocyte, macrophage, or platelet. Other cells include a bone marrow stromal cell (mesenchymal stem cell) or a cell that develops therefrom such as a bone cell (osteocyte), cartilage cells (chondrocyte), fat cell (adipocyte), or other kinds of connective tissue cells such as one found in tendons; neural stem cell or a cell it gives rise to including, for example, a nerve cells (neuron), astrocyte or oligodendrocyte; epithelial stem cell or a cell that arises from an epithelial stem cell such as an absorptive cell, goblet cell, Paneth cell, or enteroendocrine cell; skin stem cell; epidermal stem cell; or follicular stem cell. Generally any type of stem cell can be used including, without limitation, an embryonic stem cell, adult stem cell, or pluripotent stem cell.

A cell from which a target nucleic acid sample is obtained for use in the invention can be a normal cell or a cell displaying one or more symptoms of a particular inflammatory condition or immunological condition such as an autoimmune disease. Those skilled in the art will know or be able to readily determine methods for isolating target nucleic acid, such as gDNA, from a cell, fluid or tissue using methods known in the art such as those described in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998).

A method of the invention can further include steps of isolating a particular type of cell or tissue. Exemplary methods that can be used in a method of the invention to isolate a particular cell from other cells in a population include, but are not limited to, Fluorescent Activated Cell Sorting (FACS) as described, for example, in Shapiro, Practical Flow Cytometry, 3rd edition Wiley-Liss; (1995), density gradient centrifugation, or manual separation using micromanipulation methods with microscope assistance. Exemplary cell separation devices that are useful in the invention include, without limitation, a Beckman JE-6 centrifugal elutriation system, Beckman Coulter EPICS ALTRA computer-controlled Flow Cytometer-cell sorter, Modular Flow Cytometer from Cytomation, Inc., Coulter counter and channelyzer system, density gradient apparatus, cytocentrifuge, Beckman J-6 centrifuge, EPICS V dual laser cell sorter, or EPICS PROFILE flow cytometer. A tissue or population of cells can also be removed by surgical techniques.

A gDNA or other target nucleic acid can be prepared for use in a method of the invention by lysing a cell that contains the nucleic acid. Typically, a cell is lysed under conditions that substantially preserve the integrity of the nucleic acid. In particular, exposure of a cell to alkaline pH can be used to lyse a cell in a method of the invention while causing relatively little damage to gDNA. Cells lacking a cell wall either naturally or due to enzymatic removal can also be lysed by exposure to osmotic stress. Other conditions that can be used to lyse a cell include exposure to detergents, mechanical disruption, sonication, heat, pressure differential such as in a French press device, or Dounce homogenization.

In particular embodiments of the invention, a crude cell lysate containing a target nucleic acid such as gDNA can be directly replicated, amplified or detected without further isolation of the target nucleic acid. Alternatively, a target nucleic acid can be further isolated from other cellular components prior to replication, amplification or detection. Accordingly, a method of the invention can be carried out on purified or partially purified target nucleic acid. Genomic DNA can be isolated using known methods including, for example, liquid phase extraction, precipitation, solid phase extraction, chromatography and the like. Such methods are often referred to as minipreps and are described for example in Sambrook et al., supra, (2001) or in Ausubel et al., supra, (1998) or available from various commercial vendors including, for example, Qiagen (Valencia, Calif.) or Promega (Madison, Wis.).

A method of the invention can include a step of amplifying a nucleic acid target including, for example, a step of whole genome amplification. Two exemplary approaches to whole genome amplification that can be used in the invention include the use of some form of randomly-primed amplification or creation of a genomic representation amplifiable by universal PCR. Exemplary techniques for randomly-primed amplification include, without limitation, those based upon PCR, such as PEP-PCR or DOP-PCR or those based upon strand-displacement amplification such as random-primer amplification. An exemplary method of creating genomic representations amplifiable by universal PCR is described, for example, in Lucito et al., Proc. Nat'l. Acad. Sci. USA 95:4487-4492 (1998). One implementation of genomic representations is to create short genomic inserts (for example, 30-2000 bases) via restriction digestion of gDNA, and add universal PCR tails by adapter ligation. Further methods of amplification are described, for example, in U.S. Ser. No. 10/600,634, filed Jun. 20, 2003.

A variety of molecular methods useful for determining the presence or absence of one or more SNPs that make up a haplotype are well known in the art. One skilled in the art understands that, where the presence or absence of a particular haplotype is to be determined, the SNPs of the haplotype can be detected by any combination of molecular methods.

In particular embodiments, a haplotype can be determined by detecting a plurality of SNPs in a target nucleic acid sample using an array. As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in Butte, Nature Reviews Drug Discov. 1:951-60 (2002) or U.S. Pat Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

In particular embodiments, probes useful in detecting target nucleic acids can be attached to particles that are arrayed or otherwise spatially distinguished. Exemplary particles include microspheres or beads. It will be understood that particles such as microspheres or beads can be spherical or approximately spherical but need not be perfectly spherical. Rather solid phase particles having other shapes including, but not limited to, cylinders disks, plates, chips, slivers or irregular shapes can be used. In addition, particles used in the invention can be porous, thus increasing the surface area available for attachment or detection of molecules such as probes or targets. Particle sizes can range, for example, from nanometers such as about 100 nm beads, to millimeters, such as about 1 mm beads, with particles of intermediate size such as at most about 0.2 micron, 0.5 micron, 5 micron or 200 microns being useful. The composition of the beads can vary depending, for example, on the application of the invention or the method of synthesis. Typically, useful particles consist of a substantially non-compressible or inelastic material compared to a biological cell such as plastics, ceramics, glass, polystyrene, methylstyrene, acrylic polymers, paramagnetic materials, thoria sol, carbon graphite, titanium dioxide, latex, Teflon™, cross-linked dextrans such as Sepharose™, cellulose, or nylon. However, if desired a biological cell or similarly compressible particle such as a cross-linked micelle can be used as a solid phase support in the invention. Other suitable bead compositions include, but are not limited to, those used in peptide, nucleic acid and organic moiety synthesis or others described, for example, in Microsphere Detection Guide from Bangs Laboratories, Fishers Ind.

Several embodiments including the use of array-based detection in the invention are exemplified below for beads or microspheres. Exemplary bead-based arrays that can be used in the invention include, without limitation, those in which beads are associated with a solid support such as those described in U.S. Pat. No. 6,355,431 B1, US 2002/0102578 and PCT Publication No. WO 00/63437. Beads can be located at discrete locations, such as wells, on a solid-phase support, whereby each location accommodates a single bead. Alternatively, discrete locations where beads reside can each include a plurality of beads as described, for example, in U.S. patent application Nos. 2004/0263923, 2004/0233485, US 2004/0132205, or US 2004/0125424. Beads can be associated with discrete locations via covalent bonds or other non-covalent interactions such as gravity, magnetism, ionic forces, van der Waals forces, hydrophobicity or hydrophilicity. However, the sites of an array of the invention need not be discrete sites. For example, it is possible to use a uniform surface of adhesive or chemical functionalities that allows the attachment of particles at any position. Thus, the surface of an array substrate can be modified to allow attachment or association of microspheres at individual sites, whether or not those sites are contiguous or non-contiguous with other sites. Thus, the surface of a substrate can be modified to form discrete sites such that only a single bead is associated with the site or, alternatively, the surface can be modified such that a plurality of beads populates each site.

Beads or other particles can be loaded onto array supports using methods known in the art such as those described, for example, in U.S. Pat. No. 6,355,431. In some embodiments, for example when chemical attachment is done, particles can be attached to a support in a non-random or ordered process. For example, using photoactivatible attachment linkers or photoactivatible adhesives or masks, selected sites on an array support can be sequentially activated for attachment, such that defined populations of particles are laid down at defined positions when exposed to the activated array substrate. Alternatively, particles can be randomly deposited on a substrate. In embodiments where the placement of probes is, random, a coding or decoding system can be used to localize and/or identify the probes at each location in the array. This can be done in any of a variety of ways, for example, as described in U.S. Pat. No. 6,355,431 or WO 03/002979. A further encoding system that is useful in the invention is the use of diffraction gratings as described, for example, in U.S. patent application Nos. US 2004/0263923, US 2004/0233485, US 2004/0132205, or US 2004/0125424.

An array of beads useful in the invention can also be in a fluid format such as a fluid stream of a flow cytometer or similar device. Exemplary formats that can be used in the invention to distinguish beads in a fluid sample using microfluidic devices are described, for example, in U.S. Pat. No. 6,524,793. Commercially available fluid formats for distinguishing beads include, for example, those used in xMAP™ technologies from Luminex or MPSS™ methods from Lynx Therapeutics.

Any of a variety of arrays known in the art can be used in the present invention. For example, arrays that are useful in the invention can be non-bead-based. A particularly useful array is an Affymetrix® GeneChip® array. GeneChip® arrays can be synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. Pat. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Applications Nos. PCT/US99/00730 (International Publication No. WO 99/36760) and PCT/US01/04285.

A spotted array can also be used in a method of the invention. An exemplary spotted array is a CodeLink™ Array available from Amersham Biosciences. CodeLink™ Activated Slides are coated with a long-chain, hydrophilic polymer containing amine-reactive groups. This polymer is covalently crosslinked to itself and to the surface of the slide. Probe attachment can be accomplished through covalent interaction between the amine-modified 5′ end of the oligonucleotide probe and the amine reactive groups present in the polymer. Probes can be attached at discrete locations using spotting pens. Useful pens are stainless steel capillary pens that are individually spring-loaded. Pen load volumes can be less than about 200 nL with a delivery volume of about 0.1 nL or less. Such pens can be used to create features having a spot diameter of, for example, about 140-160 μm. In a preferred embodiment, nucleic acid probes at each spotted feature can be 30 nucleotides long. However, probes having other lengths such as those set forth elsewhere herein can also be attached at each spot.

An array that is useful in the invention can also be manufactured using inkjet printing methods such as SurePrint™ Technology available from Agilent Technologies. Such methods can be used to synthesize oligonucleotide probes in situ or to attach pre-synthesized probes having moieties that are reactive with a support surface. A printed microarray can contain 22,575 features on a surface having standard slide dimensions (about 1 inch by 3 inches). Typically, the printed probes are 25 or 60 nucleotides in length. However, probes having other lengths such as those set forth elsewhere herein can also be printed at each location.

An exemplary high density array is an array of arrays or a composite array having a plurality of individual arrays that is configured to allow processing of multiple samples. Such arrays allow multiplex detection. Exemplary composite arrays that can be used in the invention, for example, in multiplex detection formats are described in U.S. Pat. No. 6,429,027 and US 2002/0102578. In particular embodiments, each individual array can be present within each well of a microtiter plate. Thus, depending on the size of the microtiter plate and the size of the individual array, very high numbers of assays can be run simultaneously; for example, using individual arrays of 2,000 probes and a 96 well microtiter plate, 192,000 assays can be performed in parallel; the same number of probes in each well of a 384 microtiter plate yields 768,000 simultaneous assays, and in a 1536 microtiter plate gives 3,072,000 assays.

In some embodiments, solid-phase attached probes, such as those having sequences set forth elsewhere herein, can be synthesized by sequential addition of monomer units directly on a solid support such as a bead or slide surface. Methods known in the art for synthesis of a variety of different chemical compounds on solid supports can be used in the invention, such as methods for solid-phase synthesis of peptides, organic moieties, and nucleic acids. Alternatively probes can be synthesized first, and then covalently attached to a solid support, for example, via reactive functional groups. Functionalized solid supports can be produced by methods known in the art or, if desired, obtained from any of several commercial suppliers. Exemplary surface chemistries that are useful in the invention include, but are not limited to, amino groups such as aliphatic and aromatic amines, carboxylic acids, aldehydes, amides, chloromethyl groups, hydrazide, hydroxyl groups, sulfonates or sulfates. If desired, a probe can be attached to a solid support via a chemical linker. Such a linker can have characteristics that provide, for example, stable attachment, reversible attachment, sufficient flexibility to allow desired interaction with a target molecule to be detected, or to avoid undesirable binding reactions. Further exemplary methods that can be used in the invention to attach polymer probes to a solid support are described in U.S. patent application Ser. No. 10/651,568 or Ser. No. 10/739,959; WO 01/41918; WO 04/001646; Pease et al., Proc. Natl. Acad. Sci. USA 91(11):5022-5026 (1994); Khrapko et al., Mol Biol (Mosk) (USSR) 25:718-730 (1991); Stimpson et al., Proc. Natl. Acad. Sci. USA 92:6379-6383 (1995) or Guo et al., Nucleic Acids Res. 22:5456-5465 (1994).

Very high density arrays are useful in the invention including, for example, those having from about 10,000,000 array locations/cm² to about 2,000,000,000 array locations/cm² or from about 100,000,000 array locations/cm² to about 1,000,000,000 array locations/cm². High density arrays can also be used including, for example, those in the range from about 100,000 array locations/cm² to about 10,000,000 array locations/cm² or about 1,000,000 array locations/cm² to about 5,000,000 array locations/cm². Moderate density arrays useful in the invention can range from about 10,000 array locations/cm² to about 100,000 array locations/cm², or from about 20,000 array locations/cm² to about 50,000 array locations/cm². Low density arrays are generally less than 10,000 particles/cm² with from about 1,000 array locations/cm² to about 5,000 array locations/cm² being useful in particular embodiments. Very low density arrays having less than 1,000 array locations/cm², from about 10 array locations/cm² to about 1000 array locations/cm², or from about 100 array locations/cm² to about 500 array locations/cm² are also useful in some applications.

A solid-phase support used in an array of the invention can be made from any material that can be modified to contain discrete individual sites or to attach a desired probe. In embodiments where arrays of particles are used, a material that is capable of attaching or associating with one or more type of particles can be used. Useful supports include, but are not limited to, glass; modified glass; functionalized glass; plastics such as acrylics, polystyrene and copolymers of styrene and other materials, polypropylene, polyethylene, polybutylene, polyurethanes, Teflon, or the like; polysaccharides; nylon; nitrocellulose; resins; silica; silica-based materials such as silicon or modified silicon; carbon; metal; inorganic glass; optical fiber bundles, or any of a variety of other polymers. Useful supports include those that allow optical detection, for example, by being translucent to energy of a desired detection wavelength and/or do not themselves appreciably fluoresce at particular detection wavelengths.

The surface of a solid-phase support can include a plurality of individual arrays that are physically separated from each other. For example, physical separation can be due to the presence of assay wells, such as in a microtiter plate. Other barriers that can be used to physically separate array locations include, for example, gaskets, raised barriers, channels, hydrophobic regions that will deter flow of aqueous solvents or hydrophilic regions that will deter flow of apolar or hydrophobic solvents.

Arrays that are physically separated from each other provide separate assay locations. An assay location can include an array of probes and provide a vessel for holding a fluid such that the fluid contacts the probes. For example, an assay location containing a multiplex HLA allele detection reaction can be contacted with an array of probes under hybridization conditions set forth herein or known in the art. Similarly, a wash fluid or fluid containing other reagents or analytes described herein can be contacted with an array of probes when placed in an assay location. An assay location can be enclosed, if desired, for example, to form a hybridization chamber. Exemplary enclosures include, without limitation, a cassette, enclosed well, or a slide surface enclosed by a gasket or membrane or both. Further exemplary enclosures that are useful in the invention are described in WO 02/00336, U.S. patent application Pub. 02/0102578 or the references cited previously herein in regard to different types of arrays.

In a particular embodiment, an array support can be an optical fiber bundle or array, as is generally described in U.S. Ser. No. 08/944,850, U.S. Pat. No. 6,200,737; WO 98/40726, and WO 98/50782. Also useful in the invention is a preformed unitary fiber optic array having discrete individual fiber optic strands that are co-axially disposed and joined along their lengths. A distinguishing feature of a preformed unitary fiber optic array compared to other fiber optic formats is that the fibers are not individually physically manipulable; that is, one strand generally cannot be physically separated at any point along its length from another fiber strand.

Any of a variety of conditions can be used to hybridize probes with target nucleic acids having SNPs. In particular embodiments, the hybridization conditions can support modification or replication of the probe, target or both. Accordingly, the presence of a particular SNP can be determined based on a detectable property of a target nucleic acid bearing the SNP, a probe that binds to the target nucleic acid or both.

In particular embodiments, SNPs can be detected based on the presence of the probe, SNP bearing target or both in a hybrid, without subsequent modification of the hybrid species. For example, a pre-labeled gDNA fragment having a particular SNP can be identified based on presence of the label at a particular array location where a nucleic acid complement of the SNP resides.

Alternatively, arrayed nucleic acid probes can be modified while hybridized to target nucleic acids, thereby allowing detection. Such embodiments include, for example, those utilizing allele-specific oligonucleotide hybridization, allele-specific primer extension (ASPE), single base extension (SBE), oligonucleotide ligation amplification (OLA), rolling circle amplification (RCA), extension ligation (GoldenGate™), invader technology, probe cleavage or pyrosequencing as described in U.S. Pat. No. 6,355,431 B1 or U.S. Ser. No. 10/177,727. Thus, the invention can be carried out in a mode wherein an immobilized probe is modified instead of a target nucleic acid captured by a probe. Alternatively, detection can include modification of the target nucleic acid while hybridized to probes. Exemplary modifications include those that are catalyzed by an enzyme such as a polymerase. A useful modification can be incorporation of one or more nucleotides or nucleotide analogs to a primer hybridized to a template strand, wherein the primer can be either the probe or target in a probe-target hybrid. Such a modification can include replication of all or part of a primed template.

In one embodiment, a haplotype is associated with a particular condition with an odds ratio of at least 5 and a lower 95% confidence limit of greater than 1. Such an odds ratio can be, for example, at least 5.5, 6.0, 6.5, 7.0, 7.5 or 8.0 with a lower 95% confidence interval limit of greater than 1, such as an odds ratio of at least 8 with a 95% confidence interval of 1.55 to 100. In some cases, it is known that a confidence interval can be higher than 100. Methods for determining an odds ratio are well known in the art (see, for example, Schlesselman et al., Case Control Studies: Design, Conduct and Analysis Oxford University Press, New York (1982).

In another embodiment, a SNP or haplotype is associated with a condition, such as an autoimmune disease, with a p value of equal to or less than 0.0097. As used herein the term “p value” is synonymous with “probability value.” The expected p value for the association between a random haplotype (or allele) and disease is 1.00. A p value of less than 0.05 indicates that haplotype and disease do not appear together by chance but are influenced by positive factors. A disease-associated haplotype or disease-associated allele can be associated with an immunological or inflammatory disease with, for example, a p value of 0.04, 0.03, 0.02, 0.01, 0.009, 0.008, 0.007, 0.006, 0.005, 0.004, 0.003, 0.002 or 0.001, or with a p value of 0.00095, 0.0009, 0.00085 or 0.0008. It is recognized that, in some cases, p values may need to be corrected, for example, to account for factors such as sample size (number of families, cases or controls), genetic heterogeneity (linked forms and unlinked forms within the particular disease), clinical heterogeneity, or analytical approach (parametric or nonparametric method).

The invention also provides a composition including a plurality of nucleic acid probes specific for at least one SNP, wherein at least one of the oligonucleotides has a sequence that is complementary to the at least one SNP. In particular embodiments, the plurality of SNPs can include one or more of the SNPs listed in Table 1. Table 2 lists exemplary probes that are useful for detecting the SNPs listed in Table 1 using, for example, a GoldenGate™ assay format. The composition can further include a nucleic acid probe that is specific for another genetic locus such as a causal variant or one or more others set forth above.

A nucleic acid probe that is complementary to a SNP locus can have any length sufficient to specifically hybridize to a desired SNP in a genomic DNA sample. Exemplary lengths include at least about 10, 15, 18, 20, 23, 25, 30, 35, 40, 45, 50, 55, 60, 65, 70, 75, 80, 85, 90, 95 or 100 nucleotides or more. A nucleic acid probe useful in the invention can have a sequence that is complementary to sequences flanking one or both sides of a SNP locus in a gDNA or amplification product derived from a gDNA. In particular embodiments, a SNP can be identified by hybridization of two or more probes. For example, an oligo ligation assay or GoldenGate™ assay can utilize probes that hybridize to separate portions of the sequence around or over a SNP such that identification of the SNP is based on ligation of the two probes as described, for example, in U.S. Pat. No. 6,355,431 B1 and U.S. Ser. No. 10/177,727. Similarly, PCR methods can be carried out using amplification primers that hybridize to sequences that flank the desired SNP and occur on opposite strands. Thus, a SNP probe of the invention can have 10, 15, 18, 20, 23, 25, 30, 35, 40, 45, 50, 55, 60, 65, 70, 75, 80, 85, 90, 95 or 100 nucleotides that are complementary to a flanking sequence of a SNP.

A nucleic acid useful in the present invention will generally contain phosphodiester bonds, and can include, for example, DNA or RNA. If desired to suit a particular application, polynucleotide analogs having alternate backbones can be used, including, for example, phosphoramide (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 1 11:2321 (1989), O-methylphophoroamidite linkages (see Eckstein, Oligonucleotides and Analogues: A Practical Approach, Oxford University Press), or peptide nucleic acid linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other polynucleotide analogs include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. polynulceotides containing one or more carbocyclic sugars can also be used in the invention (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. If desired, these modifications of the ribose-phosphate backbone can be included in a polynucleotide to facilitate the addition of labels, or to increase the stability and half-life of such molecules in physiological environments.

A nucleic acid useful in the invention can be an isolated nucleic acid prepared using methods well known in the art such that it is removed from at least one coexisting biological component. A nucleic acid can also be synthesized using any of a variety of methods well known in the art including, for example, a method described in U.S. Pat. Nos. 6,121,054 and 6,663,832; WO 00/44491 and U.S. Ser. No. 10/651568.

A nucleic acid can be single stranded, double stranded or contain portions of both double stranded and single stranded sequence. A nucleic acid can be DNA, such as genomic DNA (gDNA) or copy DNA (cDNA); RNA such as messenger RNA (mRNA), transfer RNA (tRNA), ribosomal RNA (rRNA), antisence RNA (aRNA) or RNA inhibitor (RNAi); or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, or the like. Particular embodiments utilize isocytosine and isoguanine as is generally described in U.S. Pat. No. 5,681,702.

In particular embodiments, a nucleic acid probe useful in the invention can include a detection moiety. A detection moiety can be a primary label that is directly detectable or secondary label that can be indirectly detected, for example, via direct or indirect interaction with a primary label. Exemplary primary labels include, without limitation, an isotopic label such as a naturally non-abundant radioactive or heavy isotope; chromophore; luminophore; fluorophore; calorimetric agent; magnetic substance; electron-rich material such as a metal; electrochemiluminescent label such as Ru(bpy)32+; or moiety that can be detected based on a nuclear magnetic, paramagnetic, electrical, charge to mass, or thermal characteristic. Fluorophores that are useful in the invention include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dye, phycoerythin, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066. Labels can also include enzymes such as horseradish peroxidase or alkaline phosphatase or particles such as magnetic particles or optically encoded nanoparticles.

Exemplary secondary labels are binding moieties. A binding moiety can be attached to a nucleic acid to allow detection or isolation of the nucleic acid via specific affinity for a receptor. Specific affinity between two binding partners is understood to mean preferential binding of one partner to another compared to binding of the partner to other components or contaminants in the system. Binding partners that are specifically bound typically remain bound under the detection or separation conditions described herein, including wash steps to remove non-specific binding. Depending upon the particular binding conditions used, the dissociation constants of the pair can be, for example, less than about 10⁻⁴, 10⁻⁵, 10⁻⁶, 10⁻⁷, 10⁻⁸, 10⁻⁹, 10⁻¹⁰, 10⁻¹¹, or 10⁻¹² M⁻¹.

Exemplary pairs of binding moieties and receptors that can be used in the invention include, without limitation, antigen and immunoglobulin or active fragments thereof, such as FAbs; immunoglobulin and immunoglobulin (or active fragments, respectively); avidin and biotin, or analogs thereof having specificity for avidin such as imino-biotin; streptavidin and biotin, or analogs thereof having specificity for streptavidin such as imino-biotin; carbohydrates and lectins; and other known proteins and their ligands. It will be understood that either partner in the above-described pairs can be attached to a nucleic acid and detected or isolated based on binding to the respective partner. It will be further understood that several moieties that can be attached to a nucleic acid can function as both primary and secondary labels in a method of the invention. For example, strepatvidin-phycoerythrin can be detected as a primary label due to fluorescence from the phycoerythrin moiety or it can be detected as a secondary label due to its affinity for anti-streptavidin antibodies, as set forth in U.S. Pat. No. 6,203,989.

In a particular embodiment, the secondary label can be a chemically modifiable moiety. In this embodiment, labels having reactive functional groups can be incorporated into a nucleic acid. The functional group can be subsequently covalently reacted with a primary label. Suitable functional groups include, but are not limited to, amino groups, carboxy groups, maleimide groups, oxo groups and thiol groups.

Binding moieties can be particularly useful when attached to primers or nucleotide triphosphates used for replication or amplification of a gDNA because the resulting population of genome fragments can be attached to an array via said binding moieties. Furthermore, binding moieties can be useful for separating replicated or amplified genome fragments from other components of an amplification reaction, concentrating the genome fragments, or detecting one or more members of a population of genome fragments when bound to capture probes on an array.

A binding moiety, detection moiety or any other useful moiety can be attached to a nucleic acid such as an amplified genome fragment using methods known in the art. For example, a primer used to amplify a nucleic acid can include the moiety attached to a base, ribose, phosphate, or analogous structure in a nucleic acid or analog thereof. In particular embodiments, a moiety can be incorporated using modified nucleosides that are added to a growing nucleotide strand, for example, during a replication, amplification or detection step. Nucleosides can be modified, for example, at the base or the ribose, or analogous structures in a nucleic acid analog.

### EXAMPLES

### Example 1

### SNP Selection

This example describes identification of candidate SNPs in the MHC region for use in creating a haplotype map. This example also describes design of probe nucleic acids useful for detecting SNPs in the MHC region.

The physical region for the MHC region was determined to be the region of chromosome 6 that is flanked by the genes RFP to MLN. Based on the build33 genome assembly the region spanned from 28,933,330 bp (RFP start) to 33,773,208 bp (MLN end). This region was extended by adding the 10 kb flanking on either side so the region used to find SNPs spanned from 28,923,330 bp to 33,783,208 bp of chromosome 6. The dbSNP database (build 116) was queried for all SNPs in the 28,924,391 bp to 33,389,103 bp region. All SNPs that were classified as two-hit, JSNP, Illumina-verified, or Perlegen-verified were added to the first SNP panel. A second SNP panel was created based on a refined search using the criteria of the first search and further requiring that included SNPs were either in or within 5 kb of coding sequence in genes in the MHC region (defined as 28,923,330 bp to 33,783,208 bp). The SNPs were evaluated using proprietary oligo design software as follows. Candidate SNPs first underwent an informatic screen for suitability for assay design. SNPs were excluded from assay design if they are located near or within palindromic sequences, GT- or AT-rich regions or repeats similar to sequences elsewhere in the genome. SNPs with design score of >0.40 on the best strand and >0.10 on the worst stand were maintained in the second SNP panel. The second SNP panel is listed in Table 1.

Three nucleic acid probes were designed for each SNP locus that passed screening. There were two 5′ allele-specific oligonucleotides (ASOs), designed to hybridize upstream of the SNP, with the 3′ base of each being allele-specific. The third oligonucleotide is located 3′ of the two ASOs, and is a locus-specific oligonucleotide (LSO). All three oligonucleotide sequences also contain universal PCR primer sites. The LSO contains a unique address sequence that is complementary to a capture sequence on the array. The nucleic acids probes were designed for use in the GoldenGate™ assay, the details of which are known in the art (see, for example, U.S. Pat. No. 6,355,431 B1 and U.S. Ser. No. 10/177,727). The allele-specific and locus-specific portions of the ASOs and LSOs, respectively are listed in Table 2.

### Example 2

### Major Histocompatibility Complex specialty SNP Probe Panel

This example describes development of a SNP panel in the 5 Mb region of chromosome 6 encompassing the major histocompatibility complex (MHC). A high-density map in this 5 Mb region is developed using both a randomly spaced and an exon-centric SNP approach. These SNPs are genotyped in individuals with known HLA types. Haplotypes derived from the SNP panel loci are tested to demonstrate predictive power for known HLA alleles, particularly rare HLA alleles. The resulting set of SNP probes can allow researchers to determine HLA alleles by elucidating the associated SNP haplotypes.

An important point is that the classical HLA loci represent a minority of genes found in the MHC region. Therefore, it is likely that disease-causing mutations will actually reside outside one of the classical HLA genes. Therefore, since almost every immunological and inflammatory disorder is studied in this genomic region, the panel of SNP probes will also help discriminate between causal alleles and variation that is in linkage disequilibrium (LD) with causal alleles.

Nucleic acid probe sets are developed including probes that are complementary to sequences in the 5 Mb region of the MHC region on human chromosome 6. The probes are selected to hybridize to approximately 1 SNP every 1.3 kb. SNP lists for the probe panel are listed in Table 1.

Several plates are genotyped. These 2 plates include the CEPH hapmap plate (30 trios see Dausset et al. Centre d'etude du polymorphisme humain (CEPH): collaborative genetic mapping of the human genome. Genomics 6:575-577 (1990)), and a second plate with additional pedigree members from the hapmap plate plus other CEPH pedigree members. HLA typing for the samples is done using molecular typing methods. The plates have HLA typing, information about the specific HLA genes (HLA-A, HLA-B, HLA-C, HLA-DRB1, HLA-DMB1, HLA_DQA1, HLA_DQB1, HLA-DPA1, HLA-DPB1), 2 TAP genes (TAP 1, TAP2), and 10 STRs. A third plate consists of samples with known HLA typing HLA-A, -B, -C, DRB1 (SP reference panel and consanguineous reference panel). The remaining plates are derived from disease and population sample collections as outlined in Table 3 below. In order to observe at least 10 copies of each HLA allele with a population frequency >5%, it is estimated that 4000 samples would need to be genotyped. Since the sample collection has not been typed for all HLA genes, it is estimated that 4700-5000 total samples would need to be genotyped in order to detect rare HLA alleles. These samples represent a population based sampling in 3 disease collections (SLE, IDDM, MS) and multiple populations from 3 ethnic groups (Caucasian, African-American, Asian).

| Disease | No. | No. | Total | HLA typing | | | | |
|---|---|---|---|---|---|---|---|---|
| phenotype | trios | case/control | no. indiv. | HLA-A | HLA-B | HLA-C | DRB1 | DQB1 |
| MS | 500 | 500 | 2000 | | | | + | + |
| IDDM | 500 | | 1500 | + | + | + | + | + |
| SLE | 250 | | 750 | | | | + | + |
| Population | | | 1000 | + | + | + | + | + |
| sample | | | | | | | | |
| Total | | | 5250 | | | | | |

A similar method is performed in which several plates are genotyped. These plates include the CEPH HapMap plate (30 trios see Dausset et al. Centre d'etude du polymorphisme humain (CEPH): collaborative genetic mapping of the human genome. Genomics 6:575-577 (1990)), and a second plate with additional pedigree members from the hapmap plate plus other CEPH pedigree members. HLA typing is known for the samples. A third plate includes the Han Chinese (Beijing)/Japanese (Tokyo) HapMap plate and the fourth plate includes the Yoruba (Ibadan, Nigeria) HapMap plate. HLA types for all plates are done using molecular typing methods. The plates have HLA typing, information about the specific HLA genes (HLA-A, HLA-B, HLA-C, HLA-DRB1, HLA-DMB1, HLA_DQA1, HLA_DQB1, HLA-DPA 1, HLA-DPB 1).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by particular embodiments and optional features, modification and variation of the inventions embodied therein and herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

TABLE 1

| | | | Final | Final | Final Score |
|---|---|---|---|---|---|
| | | dbSNP | Best | Score | (best > 0.4, |
| SNP Name | Coordinate | Version | Score | Worst | worst > 0.1) |
| rs2395256 | 32723376 | 111.2 | 0.985 | 0.731 | 0.985 |
| rs1894411 | 32786387 | 100.2 | 0.985 | 0.706 | 0.985 |
| rs2621414 | 32736850 | 100.2 | 0.979 | 0.465 | 0.979 |
| rs1264298 | 30945235 | 110.1 | 0.975 | 0.693 | 0.975 |
| rs1264307 | 30941897 | 106.1 | 0.974 | 0.838 | 0.974 |
| rs241454 | 32566901 | 100.2 | 0.967 | 0.858 | 0.967 |
| rs3134996 | 32631405 | 103.2 | 0.967 | 0.78 | 0.967 |
| rs2294882 | 32138272 | 111.2 | 0.965 | 0.579 | 0.965 |
| rs2001100 | 32154245 | 103.2 | 0.962 | 0.817 | 0.962 |
| rs2273017 | 32108387 | 103.2 | 0.96 | 0.763 | 0.96 |
| rs3817969 | 32387138 | 107.2 | 0.958 | 0.72 | 0.958 |
| rs761189 | 32088616 | 113.2 | 0.956 | 0.828 | 0.956 |
| rs3129736 | 32456710 | 103.2 | 0.956 | 0.718 | 0.956 |
| rs3763350 | 32780534 | 107.2 | 0.955 | 0.608 | 0.955 |
| rs3135374 | 32405081 | 103.2 | 0.954 | 0.773 | 0.954 |
| rs2294880 | 32138479 | 100.2 | 0.95 | 0.776 | 0.95 |
| rs397081 | 32218641 | 87.2 | 0.948 | 0.881 | 0.948 |
| rs3021302 | 32625764 | 101.2 | 0.948 | 0.767 | 0.948 |
| rs3129871 | 32432111 | 103.2 | 0.948 | 0.668 | 0.948 |
| rs365053 | 32222014 | 80.2 | 0.947 | 0.914 | 0.947 |
| rs1264308 | 30650745 | 110.1 | 0.947 | 0.856 | 0.947 |
| rs3806156 | 32144455 | 107.2 | 0.947 | 0.834 | 0.947 |
| rs2253588 | 30960365 | 100.2 | 0.947 | 0.74 | 0.947 |
| rs2074512 | 30940059 | 106.1 | 0.945 | 0.832 | 0.945 |
| rs2076536 | 32365137 | 113.2 | 0.945 | 0.628 | 0.945 |
| rs1988563 | 32524496 | 113.2 | 0.944 | 0.792 | 0.944 |
| rs2253705 | 30960893 | 111.2 | 0.941 | 0.708 | 0.941 |
| rs1894412 | 32505586 | 113.2 | 0.939 | 0.745 | 0.939 |
| rs2071554 | 32778091 | 98.2 | 0.938 | 0.841 | 0.938 |
| rs2395264 | 32506052 | 113.2 | 0.938 | 0.812 | 0.938 |
| rs2071290 | 32164276 | 98.1 | 0.938 | 0.719 | 0.938 |
| rs885912 | 30235364 | 100.1 | 0.937 | 0.616 | 0.937 |
| rs2073717 | 31182949 | 103.2 | 0.935 | 0.809 | 0.935 |
| rs3873444 | 32453481 | 113.2 | 0.933 | 0.78 | 0.933 |
| rs261949 | 30040838 | 108.2 | 0.933 | 0.777 | 0.933 |
| rs2187818 | 32166325 | 113.2 | 0.93 | 0.839 | 0.93 |
| rs2071550 | 32725728 | 111.2 | 0.929 | 0.729 | 0.929 |
| rs1573649 | 32732825 | 98.2 | 0.927 | 0.722 | 0.927 |
| rs2395110 | 31986633 | 113.2 | 0.926 | 0.794 | 0.926 |
| rs2073046 | 32106831 | 98.2 | 0.924 | 0.773 | 0.924 |
| rs2073047 | 32106656 | 98.2 | 0.92 | 0.87 | 0.92 |
| rs743862 | 32401404 | 113.2 | 0.92 | 0.814 | 0.92 |
| rs2074488 | 31299739 | 98.2 | 0.92 | 0.698 | 0.92 |
| rs2072895 | 29462615 | 100.1 | 0.916 | 0.615 | 0.916 |
| rs2076537 | 32088392 | 103.2 | 0.915 | 0.672 | 0.915 |
| rs2072110 | 30130934 | 100.2 | 0.914 | 0.637 | 0.914 |
| rs13501 | 32786937 | 100.2 | 0.914 | 0.609 | 0.914 |
| rs2072896 | 29461898 | 98.2 | 0.912 | 0.651 | 0.912 |
| rs2581 | 32975848 | 110.2 | 0.911 | 0.772 | 0.911 |
| rs2023472 | 30136622 | 113.2 | 0.911 | 0.753 | 0.911 |
| rs2006165 | 32723575 | 111.2 | 0.91 | 0.888 | 0.91 |
| rs2395533 | 32619446 | 111.2 | 0.91 | 0.866 | 0.91 |
| rs2269703 | 30719523 | 100.2 | 0.909 | 0.582 | 0.909 |
| rs259919 | 30086297 | 100.2 | 0.908 | 0.69 | 0.908 |
| rs1265760 | 32092629 | 113.2 | 0.907 | 0.614 | 0.907 |
| rs241428 | 32805590 | 100.2 | 0.903 | 0.891 | 0.903 |
| rs3763316 | 32147503 | 107.2 | 0.9 | 0.867 | 0.9 |
| rs8084 | 32181792 | 108.2 | 0.883 | 0.81 | 0.883 |
| rs1042337 | 32906461 | 103.2 | 0.881 | 0.743 | 0.881 |
| rs2269713 | 30911885 | 100.2 | 0.878 | 0.798 | 0.878 |
| rs2239524 | 31189391 | 103.2 | 0.874 | 0.776 | 0.874 |
| rs3115569 | 31994896 | 103.2 | 0.873 | 0.817 | 0.873 |
| rs241448 | 32567442 | 110.2 | 0.871 | 0.634 | 0.871 |
| rs2071475 | 32553144 | 100.2 | 0.87 | 0.862 | 0.87 |
| rs365066 | 32976704 | 106.2 | 0.87 | 0.738 | 0.87 |
| rs2022059 | 31927246 | 100.1 | 0.865 | 0.796 | 0.865 |
| rs2071474 | 32553339 | 100.2 | 0.863 | 0.685 | 0.863 |
| rs3129745 | 32458086 | 103.1 | 0.859 | 0.854 | 0.859 |
| rs2213572 | 32721372 | 96.1 | 0.852 | 0.72 | 0.852 |
| rs2286656 | 30960370 | 100.2 | 0.849 | 0.846 | 0.849 |
| rs2070121 | 32552311 | 96.2 | 0.846 | 0.736 | 0.846 |
| rs3117098 | 32384271 | 103.2 | 0.844 | 0.811 | 0.844 |
| rs926594 | 32076127 | 113.1 | 0.842 | 0.5 | 0.842 |
| rs2621393 | 32525973 | 100.2 | 0.84 | 0.479 | 0.84 |
| rs7192 | 32431163 | 106.2 | 0.838 | 0.688 | 0.838 |
| rs14004 | 32178466 | 110.2 | 0.838 | 0.646 | 0.838 |
| rs3134943 | 32167507 | 106.1 | 0.832 | 0.728 | 0.832 |
| rs2213585 | 32183907 | 113.2 | 0.83 | 0.771 | 0.83 |
| rs2072894 | 29778275 | 98.2 | 0.83 | 0.55 | 0.83 |
| rs2286655 | 30960734 | 111.2 | 0.822 | 0.743 | 0.822 |
| rs886420 | 30650394 | 110.1 | 0.816 | 0.784 | 0.816 |
| rs2074504 | 30590962 | 110.2 | 0.811 | 0.758 | 0.811 |
| rs2301270 | 32720950 | 100.2 | 0.81 | 0.682 | 0.81 |
| rs3763310 | 32146860 | 107.2 | 0.809 | 0.459 | 0.809 |
| rs241446 | 32567724 | 110.2 | 0.808 | 0.774 | 0.808 |
| rs3129876 | 32178769 | 103.2 | 0.805 | 0.799 | 0.805 |
| rs1043483 | 30954527 | 106.2 | 0.805 | 0.553 | 0.805 |
| rs2286405 | 30033793 | 100.2 | 0.803 | 0.766 | 0.803 |
| rs2284175 | 30645903 | 100.2 | 0.802 | 0.764 | 0.802 |
| rs1362122 | 30937862 | 113.1 | 0.801 | 0.763 | 0.801 |
| rs1265155 | 30914452 | 110.2 | 0.796 | 0.749 | 0.796 |
| rs2267641 | 30926017 | 100.1 | 0.793 | 0.653 | 0.793 |
| rs2647089 | 32452325 | 100.2 | 0.785 | 0.591 | 0.785 |
| rs2240619 | 29746211 | 98.2 | 0.784 | 0.749 | 0.784 |
| rs1061808 | 32156293 | 106.2 | 0.776 | 0.712 | 0.776 |
| rs2249099 | 30140074 | 100.2 | 0.775 | 0.538 | 0.775 |
| rs1264309 | 30936707 | 106.2 | 0.773 | 0.748 | 0.773 |
| rs3763305 | 32140245 | 107.2 | 0.771 | 0.724 | 0.771 |
| rs2073724 | 31190514 | 98.2 | 0.771 | 0.658 | 0.771 |
| rs2582 | 32975998 | 100.2 | 0.765 | 0.727 | 0.765 |
| rs2074484 | 30130552 | 113.2 | 0.765 | 0.589 | 0.765 |
| rs3134945 | 32166238 | 103.1 | 0.737 | 0.6 | 0.737 |
| rs2269418 | 31950877 | 100.2 | 0.734 | 0.711 | 0.734 |
| rs3749968 | 32054719 | 107.2 | 0.73 | 0.461 | 0.73 |
| rs2856705 | 32441713 | 100.2 | 0.72 | 0.697 | 0.72 |
| rs2071283 | 32214936 | 98.2 | 0.719 | 0.718 | 0.719 |
| rs2239518 | 30926890 | 98.1 | 0.712 | 0.468 | 0.712 |
| rs2240804 | 30981694 | 111.1 | 0.711 | 0.641 | 0.711 |
| rs2076525 | 32141373 | 98.1 | 0.704 | 0.606 | 0.704 |
| rs3806157 | 32144558 | 107.2 | 0.693 | 0.47 | 0.693 |
| rs2187823 | 32465266 | 113.2 | 0.436 | 0.385 | 0.436 |
| rs3129858 | 32171277 | 103.2 | 0.969 | 0.71 | 0.969 |
| rs3135396 | 32429995 | 113.2 | 0.953 | 0.896 | 0.953 |
| rs1736917 | 29860267 | 100.2 | 0.929 | 0.727 | 0.929 |
| rs3213485 | 32719506 | 106.2 | 0.928 | 0.907 | 0.928 |
| rs184003 | 32170042 | 96.1 | 0.915 | 0.625 | 0.915 |
| rs2647086 | 32451748 | 100.2 | 0.909 | 0.81 | 0.909 |
| rs2235383 | 29758753 | 98.2 | 0.903 | 0.895 | 0.903 |
| rs3817970 | 32386989 | 107.2 | 0.884 | 0.836 | 0.884 |
| rs2213574 | 32486594 | 96.2 | 0.87 | 0.79 | 0.87 |
| rs2858857 | 32626458 | 100.2 | 0.862 | 0.778 | 0.862 |
| rs2227139 | 32184216 | 113.2 | 0.854 | 0.783 | 0.854 |
| rs709052 | 31381985 | 113.2 | 0.842 | 0.811 | 0.842 |
| rs2239519 | 30859614 | 103.2 | 0.841 | 0.816 | 0.841 |
| rs3844313 | 32636608 | 111.2 | 0.838 | 0.5 | 0.838 |
| rs2076534 | 32388166 | 103.2 | 0.828 | 0.722 | 0.828 |
| rs2074490 | 31299245 | 98.2 | 0.828 | 0.625 | 0.828 |
| rs1049033 | 29568397 | 106.2 | 0.826 | 0.758 | 0.826 |
| rs206019 | 31950998 | 87.2 | 0.818 | 0.816 | 0.818 |
| rs1265157 | 30913023 | 100.2 | 0.813 | 0.464 | 0.813 |
| rs1044043 | 32787395 | 108.2 | 0.793 | 0.787 | 0.793 |
| rs1615839 | 29830956 | 113.2 | 0.784 | 0.755 | 0.784 |
| rs2071555 | 32906566 | 98.2 | 0.779 | 0.729 | 0.779 |
| rs241441 | 32568054 | 110.2 | 0.763 | 0.689 | 0.763 |
| rs1624278 | 29861283 | 111.2 | 0.701 | 0.538 | 0.701 |
| rs2395195 | 32218746 | 114.2 | 0.44 | 0.318 | 0.44 |
| rs915667 | 29863096 | 110.2 | 0.425 | 0.327 | 0.425 |
| rs1964995 | 32468893 | 113.2 | 0.411 | 0.305 | 0.411 |
| rs3117226 | 33059106 | 103.2 | 0.997 | 0.664 | 0.997 |
| rs2858333 | 32451842 | 100.2 | 0.995 | 0.714 | 0.995 |
| rs3025644 | 29338681 | 102.2 | 0.994 | 0.647 | 0.994 |
| rs3129939 | 32107523 | 113.2 | 0.993 | 0.706 | 0.993 |
| rs2534655 | 31531601 | 100.2 | 0.993 | 0.603 | 0.993 |
| rs2144425 | 29407525 | 96.2 | 0.992 | 0.795 | 0.992 |
| rs2523969 | 29709535 | 100.2 | 0.99 | 0.684 | 0.99 |
| rs2746149 | 29206110 | 100.2 | 0.99 | 0.645 | 0.99 |
| rs1265048 | 31142215 | 87.2 | 0.99 | 0.535 | 0.99 |
| rs2535276 | 31145178 | 100.2 | 0.99 | 0.535 | 0.99 |
| rs3129267 | 33091567 | 103.2 | 0.989 | 0.688 | 0.989 |
| rs3095314 | 30860389 | 113.2 | 0.986 | 0.745 | 0.986 |
| rs2844720 | 30246475 | 100.1 | 0.986 | 0.738 | 0.986 |
| rs2294476 | 33091746 | 100.2 | 0.985 | 0.869 | 0.985 |
| rs2394517 | 29137239 | 113.2 | 0.985 | 0.658 | 0.985 |
| rs916571 | 29972713 | 103.2 | 0.984 | 0.685 | 0.984 |
| rs3129941 | 32108443 | 103.2 | 0.984 | 0.622 | 0.984 |
| rs3817659 | 31375600 | 107.2 | 0.983 | 0.847 | 0.983 |
| rs2596449 | 31497109 | 100.2 | 0.983 | 0.8 | 0.983 |
| rs176250 | 32937553 | 106.2 | 0.983 | 0.763 | 0.983 |
| rs6457116 | 29998423 | 116.1 | 0.983 | 0.76 | 0.983 |
| rs2524070 | 31303823 | 100.2 | 0.982 | 0.772 | 0.982 |
| rs2857178 | 32523545 | 100.2 | 0.982 | 0.748 | 0.982 |
| rs3218822 | 30651094 | 106.1 | 0.982 | 0.741 | 0.982 |
| rs3817981 | 32386695 | 107.2 | 0.981 | 0.584 | 0.981 |
| rs1610735 | 29783756 | 110.2 | 0.98 | 0.801 | 0.98 |
| rs2394737 | 29913757 | 100.2 | 0.979 | 0.659 | 0.979 |
| rs2239530 | 29922873 | 98.2 | 0.978 | 0.815 | 0.978 |
| rs4415152 | 32718594 | 111.2 | 0.978 | 0.767 | 0.978 |
| rs4434496 | 32449977 | 111.2 | 0.978 | 0.733 | 0.978 |
| rs3130602 | 32973654 | 103.2 | 0.977 | 0.864 | 0.977 |
| rs211456 | 33390833 | 100.2 | 0.977 | 0.794 | 0.977 |
| rs3091282 | 33058645 | 103.2 | 0.977 | 0.784 | 0.977 |
| rs4959036 | 29706893 | 111.2 | 0.975 | 0.637 | 0.975 |
| rs2524103 | 31293908 | 111.2 | 0.974 | 0.815 | 0.974 |
| rs45855 | 31960238 | 100.2 | 0.974 | 0.688 | 0.974 |
| rs3130404 | 30003008 | 103.2 | 0.974 | 0.682 | 0.974 |
| rs211453 | 33331582 | 111.2 | 0.974 | 0.675 | 0.974 |
| rs3135375 | 32405049 | 103.2 | 0.973 | 0.482 | 0.973 |
| rs3129767 | 32365732 | 103.2 | 0.972 | 0.824 | 0.972 |
| rs7744593 | 32705896 | 116.1 | 0.972 | 0.824 | 0.972 |
| rs2734979 | 29593919 | 100.1 | 0.972 | 0.763 | 0.972 |
| rs3094149 | 29908738 | 103.2 | 0.971 | 0.823 | 0.971 |
| rs2517723 | 29686026 | 100.2 | 0.971 | 0.639 | 0.971 |
| rs1632995 | 29834587 | 89.2 | 0.971 | 0.604 | 0.971 |
| rs4248143 | 29706649 | 111.2 | 0.97 | 0.769 | 0.97 |
| rs8807 | 33034298 | 116.2 | 0.97 | 0.714 | 0.97 |
| rs3130745 | 29211171 | 103.2 | 0.97 | 0.649 | 0.97 |
| rs2858331 | 32452034 | 100.2 | 0.969 | 0.733 | 0.969 |
| rs2596503 | 31379562 | 100.2 | 0.968 | 0.828 | 0.968 |
| rs2517529 | 31140748 | 100.2 | 0.968 | 0.822 | 0.968 |
| rs761188 | 32088730 | 113.2 | 0.967 | 0.876 | 0.967 |
| rs2295664 | 31691850 | 100.2 | 0.967 | 0.795 | 0.967 |
| rs2523504 | 31569582 | 100.2 | 0.967 | 0.748 | 0.967 |
| rs7381625 | 32735736 | 116.1 | 0.966 | 0.906 | 0.966 |
| rs407161 | 29581677 | 100.2 | 0.966 | 0.761 | 0.966 |
| rs3900268 | 29584138 | 108.2 | 0.966 | 0.753 | 0.966 |
| rs3117123 | 32087146 | 111.2 | 0.966 | 0.699 | 0.966 |
| rs211455 | 33329969 | 111.2 | 0.966 | 0.69 | 0.966 |
| rs7451341 | 29496370 | 116.1 | 0.966 | 0.639 | 0.966 |
| rs3095312 | 31151640 | 110.2 | 0.966 | 0.6 | 0.966 |
| rs6932266 | 32486900 | 116.1 | 0.965 | 0.864 | 0.965 |
| rs2295120 | 33062216 | 100.2 | 0.965 | 0.797 | 0.965 |
| rs458679 | 33243940 | 108.2 | 0.965 | 0.792 | 0.965 |
| rs763028 | 32723050 | 111.2 | 0.965 | 0.702 | 0.965 |
| rs1360 | 32621541 | 106.2 | 0.965 | 0.634 | 0.965 |
| rs2734574 | 31552616 | 100.2 | 0.965 | 0.62 | 0.965 |
| rs1548515 | 30448119 | 113.2 | 0.965 | 0.501 | 0.965 |
| rs1610736 | 29783781 | 110.2 | 0.964 | 0.766 | 0.964 |
| rs1573648 | 32733006 | 98.2 | 0.964 | 0.665 | 0.964 |
| rs3807035 | 30105616 | 107.2 | 0.964 | 0.636 | 0.964 |
| rs3763354 | 32780332 | 107.2 | 0.964 | 0.615 | 0.964 |
| rs2855437 | 33131677 | 100.1 | 0.964 | 0.548 | 0.964 |
| rs3117008 | 33088947 | 103.2 | 0.963 | 0.753 | 0.963 |
| rs3823342 | 29683825 | 107.2 | 0.962 | 0.887 | 0.962 |
| rs2254618 | 31486078 | 111.2 | 0.962 | 0.834 | 0.962 |
| rs1541270 | 30164530 | 113.2 | 0.962 | 0.805 | 0.962 |
| rs3817962 | 32139071 | 107.2 | 0.962 | 0.792 | 0.962 |
| rs3763313 | 32147228 | 107.2 | 0.962 | 0.647 | 0.962 |
| rs1611651 | 29909636 | 110.2 | 0.961 | 0.849 | 0.961 |
| rs2516713 | 29998004 | 100.2 | 0.961 | 0.829 | 0.961 |
| rs3869070 | 30085039 | 108.2 | 0.961 | 0.785 | 0.961 |
| rs7743412 | 32626494 | 116.1 | 0.961 | 0.692 | 0.961 |
| rs1541267 | 30164770 | 113.2 | 0.96 | 0.737 | 0.96 |
| rs2853977 | 31150062 | 110.2 | 0.96 | 0.73 | 0.96 |
| rs3891156 | 29759498 | 113.2 | 0.959 | 0.797 | 0.959 |
| rs2394607 | 29140274 | 100.2 | 0.959 | 0.769 | 0.959 |
| rs1028411 | 29138154 | 111.2 | 0.959 | 0.735 | 0.959 |
| rs2022065 | 30182232 | 103.2 | 0.958 | 0.921 | 0.958 |
| rs2294748 | 29563620 | 100.2 | 0.958 | 0.756 | 0.958 |
| rs6933541 | 32363760 | 116.1 | 0.958 | 0.668 | 0.958 |
| rs1419638 | 29423517 | 113.2 | 0.958 | 0.551 | 0.958 |
| rs13395 | 29684102 | 101.2 | 0.957 | 0.849 | 0.957 |
| rs64036 | 29298025 | 110.2 | 0.956 | 0.772 | 0.956 |
| rs727939 | 32619634 | 108.2 | 0.956 | 0.731 | 0.956 |
| rs10751 | 32904063 | 103.2 | 0.956 | 0.717 | 0.956 |
| rs3869065 | 29758232 | 113.2 | 0.956 | 0.667 | 0.956 |
| rs3094061 | 30091947 | 103.2 | 0.955 | 0.848 | 0.955 |
| rs419434 | 32980132 | 80.2 | 0.955 | 0.755 | 0.955 |
| rs429916 | 32971969 | 80.2 | 0.955 | 0.706 | 0.955 |
| rs3130603 | 32982055 | 113.2 | 0.955 | 0.684 | 0.955 |
| rs397144 | 29948685 | 80.2 | 0.955 | 0.613 | 0.955 |
| rs3873332 | 30956789 | 113.2 | 0.955 | 0.499 | 0.955 |
| rs2735052 | 29472322 | 111.2 | 0.954 | 0.95 | 0.954 |
| rs3134759 | 30985111 | 103.2 | 0.954 | 0.792 | 0.954 |
| rs2571430 | 30010216 | 100.2 | 0.954 | 0.772 | 0.954 |
| rs4713367 | 30527580 | 111.2 | 0.954 | 0.756 | 0.954 |
| rs2523774 | 29580336 | 100.2 | 0.954 | 0.737 | 0.954 |
| rs2233969 | 31141238 | 100.2 | 0.954 | 0.633 | 0.954 |
| rs522162 | 31690675 | 113.2 | 0.954 | 0.55 | 0.954 |
| rs3763324 | 32432687 | 107.2 | 0.953 | 0.875 | 0.953 |
| rs1033568 | 29028722 | 113.2 | 0.953 | 0.85 | 0.953 |
| rs1632984 | 29835963 | 111.2 | 0.953 | 0.814 | 0.953 |
| rs1362060 | 29142170 | 113.2 | 0.953 | 0.807 | 0.953 |
| rs7754443 | 31313555 | 116.1 | 0.953 | 0.776 | 0.953 |
| rs2395351 | 33101874 | 100.2 | 0.953 | 0.633 | 0.953 |
| rs2267644 | 32218584 | 100.1 | 0.953 | 0.61 | 0.953 |
| rs29232 | 29382189 | 102.2 | 0.953 | 0.526 | 0.953 |
| rs1633085 | 29486755 | 113.2 | 0.952 | 0.863 | 0.952 |
| rs7743506 | 32626391 | 116.1 | 0.952 | 0.84 | 0.952 |
| rs1737009 | 29820484 | 89.2 | 0.952 | 0.819 | 0.952 |
| rs2844606 | 31307723 | 100.2 | 0.952 | 0.734 | 0.952 |
| rs2394173 | 29835729 | 111.2 | 0.952 | 0.675 | 0.952 |
| rs4392759 | 30179479 | 113.2 | 0.951 | 0.938 | 0.951 |
| rs3132528 | 31192375 | 103.2 | 0.951 | 0.857 | 0.951 |
| rs3130782 | 30685601 | 103.2 | 0.951 | 0.848 | 0.951 |
| rs379221 | 30010522 | 100.2 | 0.951 | 0.827 | 0.951 |
| rs7195 | 32432022 | 108.2 | 0.951 | 0.795 | 0.951 |
| rs7383433 | 32780065 | 116.1 | 0.951 | 0.696 | 0.951 |
| rs1632854 | 30746407 | 89.2 | 0.951 | 0.677 | 0.951 |
| rs5007259 | 32398570 | 113.2 | 0.95 | 0.834 | 0.95 |
| rs3130377 | 30094151 | 103.1 | 0.95 | 0.759 | 0.95 |
| rs1419632 | 29144004 | 113.2 | 0.95 | 0.716 | 0.95 |
| rs3819721 | 32798211 | 107.2 | 0.95 | 0.55 | 0.95 |
| rs2071363 | 33034582 | 98.2 | 0.949 | 0.87 | 0.949 |
| rs2233966 | 31141665 | 113.2 | 0.949 | 0.854 | 0.949 |
| rs2621416 | 32736659 | 100.2 | 0.949 | 0.821 | 0.949 |
| rs1736912 | 29860522 | 98.2 | 0.949 | 0.793 | 0.949 |
| rs3763347 | 32816388 | 111.2 | 0.949 | 0.768 | 0.949 |
| rs1610613 | 29776202 | 111.2 | 0.949 | 0.557 | 0.949 |
| rs3135373 | 32405129 | 103.2 | 0.948 | 0.904 | 0.948 |
| rs2523946 | 30002326 | 100.2 | 0.948 | 0.889 | 0.948 |
| rs3873374 | 31310603 | 113.2 | 0.948 | 0.826 | 0.948 |
| rs17034 | 32567278 | 110.2 | 0.948 | 0.818 | 0.948 |
| rs2073149 | 29427860 | 98.2 | 0.948 | 0.754 | 0.948 |
| rs2073150 | 29427678 | 98.2 | 0.948 | 0.646 | 0.948 |
| rs3117007 | 33089187 | 103.2 | 0.947 | 0.8 | 0.947 |
| rs3094207 | 31152212 | 110.2 | 0.947 | 0.731 | 0.947 |
| rs3748082 | 33284574 | 107.2 | 0.946 | 0.834 | 0.946 |
| rs423808 | 30009503 | 100.2 | 0.946 | 0.814 | 0.946 |
| rs445150 | 29709403 | 100.2 | 0.946 | 0.748 | 0.946 |
| rs3734841 | 30030063 | 107.2 | 0.945 | 0.918 | 0.945 |
| rs3132523 | 31200664 | 103.2 | 0.945 | 0.894 | 0.945 |
| rs3997854 | 32453672 | 108.2 | 0.945 | 0.762 | 0.945 |
| rs2071482 | 32810113 | 111.1 | 0.945 | 0.727 | 0.945 |
| rs2071469 | 32778198 | 100.2 | 0.945 | 0.702 | 0.945 |
| rs2736155 | 31375957 | 113.2 | 0.945 | 0.654 | 0.945 |
| rs2524198 | 30192260 | 100.2 | 0.945 | 0.582 | 0.945 |
| rs560505 | 32032528 | 83.2 | 0.944 | 0.817 | 0.944 |
| rs2894252 | 32364952 | 113.2 | 0.944 | 0.758 | 0.944 |
| rs3094220 | 31143793 | 103.2 | 0.944 | 0.745 | 0.944 |
| rs3763355 | 32780297 | 107.2 | 0.943 | 0.888 | 0.943 |
| rs3823312 | 29707755 | 107.2 | 0.943 | 0.83 | 0.943 |
| rs4999342 | 32467580 | 113.2 | 0.943 | 0.764 | 0.943 |
| rs2517403 | 30837767 | 100.2 | 0.943 | 0.742 | 0.943 |
| rs4987086 | 31313315 | 113.2 | 0.943 | 0.641 | 0.943 |
| rs3819294 | 31384241 | 107.2 | 0.943 | 0.623 | 0.943 |
| rs909728 | 29490319 | 110.2 | 0.942 | 0.903 | 0.942 |
| rs2294477 | 33100435 | 103.2 | 0.942 | 0.8 | 0.942 |
| rs3117229 | 33057516 | 103.2 | 0.942 | 0.708 | 0.942 |
| rs3909178 | 31145020 | 113.2 | 0.942 | 0.692 | 0.942 |
| rs3116985 | 33102037 | 103.2 | 0.942 | 0.573 | 0.942 |
| rs3095320 | 31151704 | 103.2 | 0.941 | 0.795 | 0.941 |
| rs241445 | 32567829 | 110.2 | 0.941 | 0.752 | 0.941 |
| rs3828896 | 31234249 | 107.2 | 0.94 | 0.913 | 0.94 |
| rs6914995 | 33042191 | 116.1 | 0.94 | 0.841 | 0.94 |
| rs3815086 | 30174546 | 107.2 | 0.94 | 0.677 | 0.94 |
| rs3132708 | 29910075 | 103.2 | 0.939 | 0.82 | 0.939 |
| rs6457644 | 32707694 | 116.1 | 0.939 | 0.744 | 0.939 |
| rs6914849 | 33042162 | 116.1 | 0.938 | 0.805 | 0.938 |
| rs4151655 | 31687743 | 110.1 | 0.938 | 0.804 | 0.938 |
| rs6457327 | 31134836 | 116.1 | 0.938 | 0.698 | 0.938 |
| rs2647087 | 32451806 | 100.2 | 0.938 | 0.626 | 0.938 |
| rs3830060 | 32404728 | 107.2 | 0.937 | 0.876 | 0.937 |
| rs915653 | 31427805 | 113.2 | 0.937 | 0.747 | 0.937 |
| rs2524059 | 31310115 | 100.2 | 0.937 | 0.686 | 0.937 |
| rs6912673 | 29566597 | 116.1 | 0.937 | 0.647 | 0.937 |
| rs4713218 | 29428411 | 111.2 | 0.936 | 0.901 | 0.936 |
| rs3135372 | 32405258 | 103.2 | 0.936 | 0.898 | 0.936 |
| rs3807034 | 30105157 | 107.2 | 0.936 | 0.788 | 0.936 |
| rs2158285 | 30101785 | 100.2 | 0.936 | 0.77 | 0.936 |
| rs2735061 | 29756449 | 110.2 | 0.936 | 0.746 | 0.936 |
| rs204995 | 31925042 | 113.1 | 0.936 | 0.674 | 0.936 |
| rs2596501 | 31379963 | 100.2 | 0.935 | 0.915 | 0.935 |
| rs2256902 | 29711130 | 110.2 | 0.935 | 0.878 | 0.935 |
| rs2844586 | 31376773 | 113.2 | 0.935 | 0.797 | 0.935 |
| rs2293861 | 31769783 | 100.2 | 0.935 | 0.764 | 0.935 |
| rs1233408 | 29579546 | 87.2 | 0.935 | 0.687 | 0.935 |
| rs7382794 | 32735599 | 116.1 | 0.934 | 0.806 | 0.934 |
| rs3830061 | 32622186 | 107.2 | 0.934 | 0.778 | 0.934 |
| rs2524074 | 31303324 | 100.2 | 0.934 | 0.703 | 0.934 |
| rs3131025 | 29461240 | 111.2 | 0.934 | 0.675 | 0.934 |
| rs1034324 | 30139370 | 113.2 | 0.933 | 0.76 | 0.933 |
| rs3130655 | 30594468 | 113.1 | 0.933 | 0.585 | 0.933 |
| rs4713324 | 30440987 | 111.2 | 0.932 | 0.82 | 0.932 |
| rs3129888 | 32431243 | 103.2 | 0.932 | 0.799 | 0.932 |
| rs2071366 | 33034074 | 98.2 | 0.931 | 0.918 | 0.931 |
| rs2074479 | 30101798 | 98.2 | 0.931 | 0.868 | 0.931 |
| rs2516312 | 31310193 | 111.2 | 0.931 | 0.856 | 0.931 |
| rs1737078 | 29495697 | 111.2 | 0.931 | 0.748 | 0.931 |
| rs3734854 | 31139642 | 107.2 | 0.931 | 0.735 | 0.931 |
| rs6901541 | 32468022 | 116.1 | 0.931 | 0.735 | 0.931 |
| rs3916766 | 32452402 | 108.2 | 0.931 | 0.636 | 0.931 |
| rs6919972 | 33035624 | 116.1 | 0.93 | 0.859 | 0.93 |
| rs6911639 | 32979625 | 116.1 | 0.93 | 0.831 | 0.93 |
| rs6921314 | 29974620 | 116.1 | 0.93 | 0.622 | 0.93 |
| rs3135351 | 32163702 | 113.2 | 0.929 | 0.885 | 0.929 |
| rs3129299 | 32902268 | 110.2 | 0.929 | 0.854 | 0.929 |
| rs7745235 | 29975730 | 116.1 | 0.929 | 0.808 | 0.929 |
| rs3819285 | 31384496 | 107.2 | 0.929 | 0.467 | 0.929 |
| rs1233493 | 29520669 | 113.2 | 0.928 | 0.808 | 0.928 |
| rs2066744 | 32719090 | 94.2 | 0.928 | 0.763 | 0.928 |
| rs1367728 | 33036262 | 111.2 | 0.927 | 0.808 | 0.927 |
| rs2853960 | 31294818 | 100.2 | 0.927 | 0.808 | 0.927 |
| rs4151672 | 31690588 | 110.1 | 0.927 | 0.794 | 0.927 |
| rs3891157 | 29759197 | 113.2 | 0.927 | 0.765 | 0.927 |
| rs2394255 | 30010203 | 113.2 | 0.927 | 0.517 | 0.927 |
| rs3128967 | 33057544 | 103.2 | 0.926 | 0.903 | 0.926 |
| rs3130106 | 30132651 | 103.2 | 0.926 | 0.895 | 0.926 |
| rs1059231 | 33280972 | 106.2 | 0.926 | 0.884 | 0.926 |
| rs356966 | 30033385 | 100.2 | 0.926 | 0.873 | 0.926 |
| rs2394605 | 29140075 | 100.2 | 0.926 | 0.813 | 0.926 |
| rs2295118 | 33062339 | 116.2 | 0.926 | 0.767 | 0.926 |
| rs2050191 | 32364691 | 113.2 | 0.926 | 0.752 | 0.926 |
| rs427417 | 32916022 | 113.2 | 0.926 | 0.744 | 0.926 |
| rs3828808 | 32630674 | 107.2 | 0.926 | 0.685 | 0.926 |
| rs7383005 | 33092283 | 116.1 | 0.925 | 0.884 | 0.925 |
| rs6940585 | 31143210 | 116.1 | 0.925 | 0.709 | 0.925 |
| rs2294745 | 29292910 | 100.2 | 0.925 | 0.607 | 0.925 |
| rs3128854 | 29202453 | 103.2 | 0.924 | 0.876 | 0.924 |
| rs1633037 | 29513367 | 89.2 | 0.924 | 0.794 | 0.924 |
| rs3132550 | 31149814 | 110.2 | 0.924 | 0.773 | 0.924 |
| rs1610708 | 29833403 | 100.2 | 0.924 | 0.596 | 0.924 |
| rs2844766 | 30001419 | 100.2 | 0.923 | 0.756 | 0.923 |
| rs4713447 | 30933721 | 111.2 | 0.923 | 0.74 | 0.923 |
| rs4151671 | 31977724 | 110.1 | 0.923 | 0.71 | 0.923 |
| rs733840 | 29489040 | 111.2 | 0.923 | 0.591 | 0.923 |
| rs3198005 | 32806627 | 105.2 | 0.922 | 0.874 | 0.922 |
| rs2523814 | 29907528 | 100.2 | 0.922 | 0.759 | 0.922 |
| rs7755580 | 32708007 | 116.1 | 0.922 | 0.726 | 0.922 |
| rs2071364 | 33034348 | 98.2 | 0.922 | 0.661 | 0.922 |
| rs2894239 | 31986553 | 113.2 | 0.921 | 0.853 | 0.921 |
| rs3117230 | 33068623 | 103.2 | 0.921 | 0.825 | 0.921 |
| rs1628578 | 29466260 | 89.2 | 0.921 | 0.8 | 0.921 |
| rs362531 | 29592911 | 102.2 | 0.921 | 0.491 | 0.921 |
| rs6899851 | 33035560 | 116.1 | 0.92 | 0.817 | 0.92 |
| rs4084091 | 31162227 | 113.2 | 0.92 | 0.792 | 0.92 |
| rs2735097 | 29686059 | 100.2 | 0.92 | 0.777 | 0.92 |
| rs1264698 | 30127934 | 87.2 | 0.92 | 0.754 | 0.92 |
| rs2071789 | 28974628 | 98.2 | 0.92 | 0.712 | 0.92 |
| rs3093544 | 31312537 | 103.2 | 0.92 | 0.688 | 0.92 |
| rs3132130 | 30088246 | 103.2 | 0.92 | 0.659 | 0.92 |
| rs3093669 | 31605443 | 111.2 | 0.92 | 0.42 | 0.92 |
| rs2092305 | 33101636 | 113.2 | 0.919 | 0.909 | 0.919 |
| rs3128927 | 33067276 | 116.2 | 0.919 | 0.882 | 0.919 |
| rs1633009 | 29828244 | 89.2 | 0.919 | 0.878 | 0.919 |
| rs6914878 | 29825606 | 116.1 | 0.919 | 0.878 | 0.919 |
| rs8337 | 29294434 | 106.2 | 0.919 | 0.84 | 0.919 |
| rs2286712 | 31555706 | 100.2 | 0.919 | 0.674 | 0.919 |
| rs1633086 | 29781002 | 113.2 | 0.919 | 0.634 | 0.919 |
| rs2245514 | 30203213 | 100.1 | 0.919 | 0.421 | 0.919 |
| rs1265086 | 31170702 | 100.2 | 0.918 | 0.77 | 0.918 |
| rs466384 | 33247364 | 106.1 | 0.917 | 0.916 | 0.917 |
| rs3117225 | 33059158 | 103.2 | 0.917 | 0.746 | 0.917 |
| rs209473 | 32916322 | 110.2 | 0.917 | 0.642 | 0.917 |
| rs4151664 | 31691631 | 110.1 | 0.917 | 0.58 | 0.917 |
| rs909967 | 29567394 | 100.2 | 0.916 | 0.915 | 0.916 |
| rs1633013 | 29820514 | 89.2 | 0.916 | 0.897 | 0.916 |
| rs4151657 | 31688298 | 110.1 | 0.916 | 0.849 | 0.916 |
| rs1419689 | 30141346 | 88.2 | 0.916 | 0.746 | 0.916 |
| rs3757333 | 30089400 | 107.2 | 0.916 | 0.385 | 0.916 |
| rs1264541 | 30448054 | 113.1 | 0.915 | 0.789 | 0.915 |
| rs7992 | 31688925 | 106.2 | 0.915 | 0.767 | 0.915 |
| rs2523926 | 30748573 | 111.1 | 0.915 | 0.725 | 0.915 |
| rs3823419 | 31161827 | 107.2 | 0.915 | 0.498 | 0.915 |
| rs2735003 | 29579392 | 100.2 | 0.914 | 0.864 | 0.914 |
| rs3817965 | 32138684 | 111.2 | 0.914 | 0.803 | 0.914 |
| rs2076540 | 32088033 | 111.1 | 0.914 | 0.668 | 0.914 |
| rs3998799 | 29753205 | 108.1 | 0.914 | 0.666 | 0.914 |
| rs2428510 | 29593785 | 100.1 | 0.913 | 0.853 | 0.913 |
| rs3094187 | 31187786 | 103.2 | 0.913 | 0.839 | 0.913 |
| rs2076484 | 29294761 | 98.2 | 0.913 | 0.781 | 0.913 |
| rs3763349 | 32801645 | 107.2 | 0.913 | 0.776 | 0.913 |
| rs2523512 | 31565528 | 100.1 | 0.913 | 0.466 | 0.913 |
| rs2517439 | 30713687 | 100.2 | 0.912 | 0.818 | 0.912 |
| rs1419634 | 29140427 | 113.2 | 0.912 | 0.805 | 0.912 |
| rs241438 | 32568377 | 110.2 | 0.912 | 0.804 | 0.912 |
| rs2244839 | 31497087 | 111.2 | 0.912 | 0.676 | 0.912 |
| rs3135033 | 32951899 | 103.2 | 0.912 | 0.547 | 0.912 |
| rs3135388 | 32183808 | 111.2 | 0.911 | 0.906 | 0.911 |
| rs3807033 | 30104744 | 107.2 | 0.911 | 0.805 | 0.911 |
| rs4713446 | 30933597 | 111.2 | 0.91 | 0.905 | 0.91 |
| rs3115608 | 29915513 | 110.2 | 0.91 | 0.839 | 0.91 |
| rs1736994 | 29829167 | 92.2 | 0.91 | 0.736 | 0.91 |
| rs6913309 | 32365629 | 116.1 | 0.91 | 0.722 | 0.91 |
| rs6905408 | 29696267 | 116.1 | 0.91 | 0.675 | 0.91 |
| rs3128917 | 33061443 | 103.2 | 0.909 | 0.829 | 0.909 |
| rs6934041 | 32724333 | 116.1 | 0.909 | 0.772 | 0.909 |
| rs6916248 | 33090117 | 116.1 | 0.908 | 0.879 | 0.908 |
| rs5007263 | 32398451 | 113.2 | 0.908 | 0.796 | 0.908 |
| rs3819300 | 31381055 | 113.2 | 0.908 | 0.741 | 0.908 |
| rs2071471 | 32778060 | 98.2 | 0.908 | 0.681 | 0.908 |
| rs2894179 | 30837429 | 101.2 | 0.907 | 0.856 | 0.907 |
| rs2239705 | 31572126 | 100.2 | 0.907 | 0.708 | 0.907 |
| rs2301220 | 33031831 | 100.2 | 0.907 | 0.686 | 0.907 |
| rs2272904 | 33129032 | 100.2 | 0.907 | 0.601 | 0.907 |
| rs6935418 | 29566695 | 116.1 | 0.906 | 0.889 | 0.906 |
| rs5021450 | 32709454 | 116.2 | 0.906 | 0.841 | 0.906 |
| rs2857103 | 32792819 | 110.1 | 0.906 | 0.838 | 0.906 |
| rs2853953 | 31294813 | 113.2 | 0.906 | 0.833 | 0.906 |
| rs757259 | 30176314 | 113.2 | 0.906 | 0.785 | 0.906 |
| rs3135391 | 32181744 | 103.2 | 0.906 | 0.749 | 0.906 |
| rs2523606 | 31381640 | 110.2 | 0.906 | 0.475 | 0.906 |
| rs1632962 | 29454566 | 89.2 | 0.905 | 0.866 | 0.905 |
| rs3135353 | 32163634 | 113.2 | 0.905 | 0.821 | 0.905 |
| rs3130235 | 33090053 | 103.2 | 0.905 | 0.799 | 0.905 |
| rs2394606 | 29140107 | 100.2 | 0.904 | 0.903 | 0.904 |
| rs2734997 | 29580403 | 100.2 | 0.904 | 0.838 | 0.904 |
| rs3998794 | 29473437 | 108.2 | 0.904 | 0.836 | 0.904 |
| rs2844463 | 31385925 | 100.2 | 0.904 | 0.82 | 0.904 |
| rs7452233 | 31237155 | 116.1 | 0.904 | 0.805 | 0.904 |
| rs6910598 | 29907379 | 116.1 | 0.904 | 0.771 | 0.904 |
| rs3129008 | 29908952 | 103.2 | 0.904 | 0.656 | 0.904 |
| rs1150741 | 30090822 | 110.2 | 0.904 | 0.647 | 0.904 |
| rs7774452 | 32725437 | 116.1 | 0.904 | 0.618 | 0.904 |
| rs6911724 | 30085973 | 116.1 | 0.903 | 0.887 | 0.903 |
| rs2294479 | 33091060 | 116.2 | 0.903 | 0.884 | 0.903 |
| rs417374 | 29289230 | 100.2 | 0.903 | 0.808 | 0.903 |
| rs1633030 | 29516552 | 89.2 | 0.903 | 0.796 | 0.903 |
| rs2024679 | 29289670 | 113.2 | 0.903 | 0.793 | 0.903 |
| rs3116999 | 33098613 | 103.2 | 0.903 | 0.779 | 0.903 |
| rs3765605 | 29746752 | 107.2 | 0.903 | 0.766 | 0.903 |
| rs2844603 | 31310146 | 100.2 | 0.903 | 0.682 | 0.903 |
| rs2621419 | 32735797 | 100.2 | 0.903 | 0.618 | 0.903 |
| rs1362073 | 29451165 | 111.2 | 0.903 | 0.39 | 0.903 |
| rs2072634 | 31688049 | 110.1 | 0.902 | 0.884 | 0.902 |
| rs213208 | 33170725 | 96.2 | 0.902 | 0.825 | 0.902 |
| rs3956934 | 29819357 | 108.2 | 0.902 | 0.786 | 0.902 |
| rs3117227 | 33058213 | 103.2 | 0.902 | 0.772 | 0.902 |
| rs4713215 | 29426558 | 111.2 | 0.902 | 0.746 | 0.902 |
| rs1632942 | 29567398 | 100.2 | 0.902 | 0.735 | 0.902 |
| rs3129886 | 32430093 | 103.2 | 0.901 | 0.897 | 0.901 |
| rs2071798 | 32485349 | 98.2 | 0.901 | 0.877 | 0.901 |
| rs2523586 | 31386184 | 100.2 | 0.901 | 0.85 | 0.901 |
| rs3117234 | 33066972 | 103.2 | 0.901 | 0.846 | 0.901 |
| rs2071473 | 32553362 | 100.2 | 0.901 | 0.527 | 0.901 |
| rs3094041 | 30132238 | 103.2 | 0.901 | 0.512 | 0.901 |
| rs2596472 | 31199725 | 100.2 | 0.9 | 0.87 | 0.9 |
| rs2213570 | 32723076 | 96.2 | 0.9 | 0.822 | 0.9 |
| rs1736922 | 29756480 | 110.2 | 0.9 | 0.812 | 0.9 |
| rs3129269 | 33099061 | 103.2 | 0.9 | 0.791 | 0.9 |
| rs6920457 | 33035846 | 116.1 | 0.899 | 0.893 | 0.899 |
| rs4711229 | 30527502 | 111.2 | 0.899 | 0.879 | 0.899 |
| rs1799964 | 31313066 | 111.2 | 0.899 | 0.836 | 0.899 |
| rs4959037 | 29707065 | 111.2 | 0.899 | 0.762 | 0.899 |
| rs1611652 | 29909922 | 100.2 | 0.899 | 0.748 | 0.899 |
| rs2071354 | 33037453 | 98.2 | 0.899 | 0.726 | 0.899 |
| rs1737012 | 29818929 | 89.2 | 0.898 | 0.805 | 0.898 |
| rs1988564 | 32524439 | 113.2 | 0.898 | 0.794 | 0.898 |
| rs2071790 | 28974354 | 103.2 | 0.898 | 0.78 | 0.898 |
| rs465151 | 33248068 | 98.1 | 0.898 | 0.775 | 0.898 |
| rs1265759 | 32093150 | 100.2 | 0.898 | 0.721 | 0.898 |
| rs2239701 | 32806569 | 110.2 | 0.898 | 0.678 | 0.898 |
| rs3129883 | 32429654 | 103.2 | 0.898 | 0.635 | 0.898 |
| rs2032502 | 28969362 | 113.2 | 0.898 | 0.549 | 0.898 |
| rs3778612 | 29821020 | 107.2 | 0.898 | 0.512 | 0.898 |
| rs2246871 | 31540049 | 100.2 | 0.897 | 0.894 | 0.897 |
| rs2239707 | 31296077 | 100.1 | 0.897 | 0.862 | 0.897 |
| rs2734916 | 29950506 | 111.2 | 0.897 | 0.794 | 0.897 |
| rs2301753 | 30100029 | 100.2 | 0.897 | 0.761 | 0.897 |
| rs2735042 | 29518131 | 100.2 | 0.897 | 0.742 | 0.897 |
| rs3179375 | 29683795 | 110.2 | 0.897 | 0.619 | 0.897 |
| rs3129869 | 32176428 | 110.2 | 0.897 | 0.536 | 0.897 |
| rs7143 | 32610390 | 52.2 | 0.896 | 0.895 | 0.896 |
| rs2068204 | 33060165 | 103.2 | 0.896 | 0.847 | 0.896 |
| rs2745411 | 29289200 | 100.2 | 0.896 | 0.779 | 0.896 |
| rs462093 | 33248795 | 96.2 | 0.896 | 0.7 | 0.896 |
| rs3817966 | 32138604 | 107.2 | 0.895 | 0.861 | 0.895 |
| rs2256919 | 30001147 | 113.2 | 0.895 | 0.832 | 0.895 |
| rs3957010 | 29703758 | 108.2 | 0.895 | 0.812 | 0.895 |
| rs1610710 | 29834298 | 100.2 | 0.895 | 0.807 | 0.895 |
| rs2735067 | 30011489 | 113.2 | 0.895 | 0.719 | 0.895 |
| rs3128963 | 33057227 | 103.2 | 0.895 | 0.654 | 0.895 |
| rs7382938 | 33091783 | 116.1 | 0.895 | 0.624 | 0.895 |
| rs3828901 | 31234476 | 107.2 | 0.894 | 0.858 | 0.894 |
| rs3131043 | 30529224 | 103.2 | 0.894 | 0.822 | 0.894 |
| rs2107197 | 29590452 | 103.1 | 0.893 | 0.89 | 0.893 |
| rs3106190 | 33282076 | 103.1 | 0.893 | 0.675 | 0.893 |
| rs1633102 | 29469579 | 89.2 | 0.892 | 0.87 | 0.892 |
| rs3117120 | 32087561 | 103.2 | 0.892 | 0.817 | 0.892 |
| rs4713610 | 33100656 | 111.2 | 0.892 | 0.799 | 0.892 |
| rs2076522 | 32141936 | 98.1 | 0.892 | 0.785 | 0.892 |
| rs2853937 | 31311861 | 111.2 | 0.892 | 0.559 | 0.892 |
| rs887464 | 30916678 | 110.2 | 0.891 | 0.768 | 0.891 |
| rs2074466 | 29470738 | 98.2 | 0.891 | 0.735 | 0.891 |
| rs7382649 | 32777623 | 116.1 | 0.891 | 0.712 | 0.891 |
| rs7452209 | 31375933 | 116.1 | 0.891 | 0.676 | 0.891 |
| rs5007238 | 29709673 | 113.2 | 0.891 | 0.547 | 0.891 |
| rs757262 | 30175727 | 113.2 | 0.89 | 0.847 | 0.89 |
| rs1611407 | 29951708 | 89.2 | 0.89 | 0.81 | 0.89 |
| rs1655937 | 29666008 | 111.2 | 0.89 | 0.8 | 0.89 |
| rs2269704 | 30718091 | 100.2 | 0.89 | 0.729 | 0.89 |
| rs2734331 | 31701109 | 110.2 | 0.89 | 0.683 | 0.89 |
| rs3998178 | 32625987 | 111.2 | 0.889 | 0.829 | 0.889 |
| rs885914 | 29973525 | 100.2 | 0.889 | 0.767 | 0.889 |
| rs6925041 | 32471768 | 116.1 | 0.888 | 0.881 | 0.888 |
| rs441126 | 32973962 | 80.2 | 0.888 | 0.781 | 0.888 |
| rs2394604 | 29140023 | 100.2 | 0.888 | 0.736 | 0.888 |
| rs2074502 | 30445036 | 98.2 | 0.887 | 0.817 | 0.887 |
| rs3815100 | 31295822 | 107.2 | 0.887 | 0.807 | 0.887 |
| rs1632932 | 29862995 | 98.2 | 0.887 | 0.773 | 0.887 |
| rs2076524 | 32141441 | 98.1 | 0.887 | 0.758 | 0.887 |
| rs7759127 | 31300296 | 116.1 | 0.886 | 0.877 | 0.886 |
| rs3765606 | 29746708 | 107.2 | 0.886 | 0.84 | 0.886 |
| rs7381988 | 31305994 | 116.1 | 0.886 | 0.836 | 0.886 |
| rs2071479 | 32551869 | 100.2 | 0.886 | 0.741 | 0.886 |
| rs4986978 | 31311158 | 113.2 | 0.886 | 0.67 | 0.886 |
| rs2071472 | 32778035 | 100.2 | 0.885 | 0.846 | 0.885 |
| rs7349863 | 29461284 | 116.1 | 0.885 | 0.783 | 0.885 |
| rs3093665 | 31604072 | 103.1 | 0.885 | 0.732 | 0.885 |
| rs3132132 | 32903414 | 103.2 | 0.885 | 0.614 | 0.885 |
| rs3823318 | 29489336 | 107.2 | 0.884 | 0.816 | 0.884 |
| rs2106072 | 29924121 | 108.2 | 0.884 | 0.724 | 0.884 |
| rs3130654 | 30593833 | 113.2 | 0.883 | 0.749 | 0.883 |
| rs2857130 | 32777950 | 100.2 | 0.882 | 0.812 | 0.882 |
| rs1264300 | 30943996 | 100.2 | 0.882 | 0.78 | 0.882 |
| rs909727 | 29490657 | 113.2 | 0.882 | 0.742 | 0.882 |
| rs4151670 | 31686290 | 110.2 | 0.882 | 0.698 | 0.882 |
| rs1419637 | 29423556 | 113.2 | 0.881 | 0.88 | 0.881 |
| rs2532934 | 30955751 | 100.2 | 0.881 | 0.722 | 0.881 |
| rs1632981 | 29836257 | 100.2 | 0.88 | 0.876 | 0.88 |
| rs7383287 | 32784620 | 116.1 | 0.88 | 0.729 | 0.88 |
| rs2071477 | 32818761 | 96.2 | 0.88 | 0.661 | 0.88 |
| rs2071652 | 29406075 | 98.2 | 0.879 | 0.81 | 0.879 |
| rs7756516 | 32725485 | 116.1 | 0.879 | 0.793 | 0.879 |
| rs2734981 | 29592450 | 110.2 | 0.879 | 0.781 | 0.879 |
| rs3823339 | 29683726 | 107.2 | 0.879 | 0.772 | 0.879 |
| rs7770557 | 30089903 | 116.1 | 0.879 | 0.727 | 0.879 |
| rs1054175 | 29915189 | 110.2 | 0.879 | 0.689 | 0.879 |
| rs3131786 | 30962671 | 103.2 | 0.878 | 0.712 | 0.878 |
| rs2844686 | 30713730 | 100.2 | 0.878 | 0.382 | 0.878 |
| rs1611653 | 29907725 | 89.2 | 0.877 | 0.876 | 0.877 |
| rs2242655 | 31686133 | 98.2 | 0.877 | 0.861 | 0.877 |
| rs969891 | 32087172 | 113.2 | 0.877 | 0.773 | 0.877 |
| rs2023609 | 30246792 | 113.2 | 0.877 | 0.734 | 0.877 |
| rs3129882 | 32429047 | 103.2 | 0.877 | 0.73 | 0.877 |
| rs3132553 | 31146003 | 113.2 | 0.877 | 0.662 | 0.877 |
| rs2233956 | 31142011 | 110.2 | 0.876 | 0.872 | 0.876 |
| rs3128930 | 33068654 | 116.2 | 0.876 | 0.822 | 0.876 |
| rs3130560 | 31161237 | 110.2 | 0.876 | 0.818 | 0.876 |
| rs1042141 | 31144272 | 106.2 | 0.876 | 0.766 | 0.876 |
| rs2256543 | 29708591 | 100.2 | 0.876 | 0.744 | 0.876 |
| rs2071362 | 33034721 | 116.2 | 0.876 | 0.659 | 0.876 |
| rs4711313 | 32973128 | 116.2 | 0.876 | 0.365 | 0.876 |
| rs916568 | 29972810 | 103.2 | 0.875 | 0.818 | 0.875 |
| rs4988889 | 32629485 | 116.2 | 0.874 | 0.596 | 0.874 |
| rs3132524 | 31200546 | 103.2 | 0.873 | 0.778 | 0.873 |
| rs1033569 | 29028527 | 113.2 | 0.873 | 0.752 | 0.873 |
| rs4713242 | 29488978 | 111.2 | 0.873 | 0.745 | 0.873 |
| rs3095334 | 30513471 | 103.2 | 0.873 | 0.555 | 0.873 |
| rs2844666 | 30747539 | 100.2 | 0.872 | 0.825 | 0.872 |
| rs2517671 | 29998375 | 102.2 | 0.872 | 0.798 | 0.872 |
| rs6903130 | 32733777 | 116.1 | 0.872 | 0.682 | 0.872 |
| rs2179919 | 33060709 | 103.2 | 0.872 | 0.604 | 0.872 |
| rs2233984 | 31140070 | 108.2 | 0.871 | 0.583 | 0.871 |
| rs3117574 | 31495988 | 103.2 | 0.871 | 0.315 | 0.871 |
| rs356969 | 29747903 | 110.2 | 0.87 | 0.669 | 0.87 |
| rs3128972 | 33060221 | 103.2 | 0.869 | 0.834 | 0.869 |
| rs4084093 | 30743923 | 108.2 | 0.869 | 0.588 | 0.869 |
| rs805262 | 31687568 | 113.2 | 0.869 | 0.517 | 0.869 |
| rs2269555 | 29027396 | 100.2 | 0.868 | 0.867 | 0.868 |
| rs3095298 | 31143738 | 103.2 | 0.868 | 0.419 | 0.868 |
| rs189865 | 32915792 | 100.2 | 0.867 | 0.853 | 0.867 |
| rs3130991 | 31151124 | 103.2 | 0.867 | 0.832 | 0.867 |
| rs1061235 | 29684056 | 108.2 | 0.867 | 0.829 | 0.867 |
| rs3823421 | 31164932 | 107.2 | 0.867 | 0.683 | 0.867 |
| rs4079877 | 28969930 | 108.2 | 0.866 | 0.81 | 0.866 |
| rs909968 | 29567362 | 113.2 | 0.866 | 0.806 | 0.866 |
| rs3218807 | 30936643 | 106.2 | 0.866 | 0.692 | 0.866 |
| rs760293 | 31382535 | 113.2 | 0.865 | 0.693 | 0.865 |
| rs5026743 | 32465722 | 113.2 | 0.864 | 0.731 | 0.864 |
| rs2213586 | 32183851 | 113.2 | 0.864 | 0.619 | 0.864 |
| rs3132709 | 29907479 | 103.2 | 0.863 | 0.764 | 0.863 |
| rs805287 | 31737414 | 113.2 | 0.863 | 0.754 | 0.863 |
| rs391755 | 32218460 | 100.1 | 0.863 | 0.686 | 0.863 |
| rs3765604 | 29746782 | 107.2 | 0.862 | 0.757 | 0.862 |
| rs1265087 | 31170630 | 100.2 | 0.862 | 0.647 | 0.862 |
| rs516535 | 32935725 | 108.2 | 0.862 | 0.538 | 0.862 |
| rs1632883 | 29686751 | 89.2 | 0.862 | 0.501 | 0.862 |
| rs7740076 | 29836080 | 116.1 | 0.862 | 0.467 | 0.862 |
| rs10885 | 31663429 | 108.2 | 0.862 | 0.448 | 0.862 |
| rs1042149 | 31143766 | 110.2 | 0.861 | 0.807 | 0.861 |
| rs3129859 | 32171696 | 103.2 | 0.861 | 0.791 | 0.861 |
| rs362546 | 29567273 | 100.2 | 0.861 | 0.776 | 0.861 |
| rs1633057 | 29497781 | 89.2 | 0.861 | 0.6 | 0.861 |
| rs3807032 | 30104568 | 107.2 | 0.861 | 0.564 | 0.861 |
| rs3130212 | 33067377 | 103.2 | 0.86 | 0.817 | 0.86 |
| rs423639 | 32981157 | 80.2 | 0.86 | 0.813 | 0.86 |
| rs3135000 | 32631272 | 103.1 | 0.859 | 0.717 | 0.859 |
| rs1264620 | 30030114 | 100.2 | 0.858 | 0.839 | 0.858 |
| rs1264703 | 29836174 | 100.2 | 0.858 | 0.521 | 0.858 |
| rs1465651 | 32902831 | 103.2 | 0.856 | 0.646 | 0.856 |
| rs7194 | 32431963 | 108.2 | 0.855 | 0.833 | 0.855 |
| rs213210 | 33168539 | 111.2 | 0.855 | 0.823 | 0.855 |
| rs2256028 | 31149956 | 110.2 | 0.855 | 0.765 | 0.855 |
| rs762815 | 32724430 | 111.2 | 0.855 | 0.712 | 0.855 |
| rs3868542 | 30916597 | 111.2 | 0.854 | 0.806 | 0.854 |
| rs5021449 | 32709373 | 116.2 | 0.854 | 0.794 | 0.854 |
| rs1632880 | 29978431 | 89.1 | 0.854 | 0.786 | 0.854 |
| rs7774954 | 32725757 | 116.1 | 0.854 | 0.717 | 0.854 |
| rs4713520 | 32282183 | 111.2 | 0.854 | 0.543 | 0.854 |
| rs6456895 | 29102443 | 116.1 | 0.852 | 0.808 | 0.852 |
| rs463302 | 33237411 | 94.1 | 0.852 | 0.717 | 0.852 |
| rs1265100 | 31166137 | 113.1 | 0.852 | 0.647 | 0.852 |
| rs3828875 | 31149926 | 107.2 | 0.852 | 0.62 | 0.852 |
| rs6917517 | 31143167 | 116.1 | 0.851 | 0.779 | 0.851 |
| rs3888777 | 30590533 | 108.2 | 0.851 | 0.683 | 0.851 |
| rs7769979 | 32725140 | 116.1 | 0.851 | 0.393 | 0.851 |
| rs3129931 | 32106273 | 103.1 | 0.85 | 0.834 | 0.85 |
| rs3868086 | 31543593 | 108.2 | 0.85 | 0.815 | 0.85 |
| rs7773068 | 32707800 | 116.1 | 0.85 | 0.798 | 0.85 |
| rs3778638 | 30862882 | 107.2 | 0.85 | 0.753 | 0.85 |
| rs3130843 | 28970766 | 103.2 | 0.85 | 0.726 | 0.85 |
| rs3885028 | 29832891 | 108.2 | 0.85 | 0.592 | 0.85 |
| rs3757327 | 30088207 | 107.2 | 0.85 | 0.541 | 0.85 |
| rs29225 | 29643493 | 102.1 | 0.849 | 0.806 | 0.849 |
| rs885937 | 29831634 | 113.2 | 0.849 | 0.797 | 0.849 |
| rs3129702 | 30003543 | 103.2 | 0.849 | 0.795 | 0.849 |
| rs3128968 | 33057700 | 103.2 | 0.849 | 0.782 | 0.849 |
| rs4713327 | 30444845 | 111.1 | 0.849 | 0.735 | 0.849 |
| rs987870 | 33035945 | 86.2 | 0.849 | 0.529 | 0.849 |
| rs7772410 | 31387604 | 116.1 | 0.848 | 0.821 | 0.848 |
| rs3998790 | 29488738 | 111.2 | 0.848 | 0.74 | 0.848 |
| rs733841 | 29783219 | 92.2 | 0.848 | 0.74 | 0.848 |
| rs6916716 | 30445949 | 116.1 | 0.848 | 0.577 | 0.848 |
| rs2395313 | 33060783 | 103.1 | 0.847 | 0.836 | 0.847 |
| rs7744001 | 32626696 | 116.1 | 0.847 | 0.827 | 0.847 |
| rs3757329 | 30089218 | 107.2 | 0.847 | 0.799 | 0.847 |
| rs6933135 | 29761679 | 116.1 | 0.847 | 0.629 | 0.847 |
| rs4713217 | 29432180 | 111.2 | 0.846 | 0.815 | 0.846 |
| rs3130198 | 33056903 | 103.2 | 0.846 | 0.679 | 0.846 |
| rs5027459 | 32720595 | 113.2 | 0.845 | 0.818 | 0.845 |
| rs2747430 | 29711023 | 100.2 | 0.844 | 0.843 | 0.844 |
| rs4248158 | 31313291 | 111.2 | 0.844 | 0.809 | 0.844 |
| rs2301227 | 33035261 | 100.2 | 0.844 | 0.775 | 0.844 |
| rs3668 | 32610448 | 36.2 | 0.844 | 0.42 | 0.844 |
| rs1233426 | 29565959 | 100.2 | 0.843 | 0.737 | 0.843 |
| rs3094093 | 30740762 | 103.2 | 0.842 | 0.809 | 0.842 |
| rs422951 | 32214410 | 113.2 | 0.842 | 0.56 | 0.842 |
| rs909729 | 29783592 | 94.2 | 0.842 | 0.491 | 0.842 |
| rs2534673 | 31236248 | 110.2 | 0.841 | 0.772 | 0.841 |
| rs6928482 | 32626859 | 116.1 | 0.841 | 0.764 | 0.841 |
| rs707938 | 31788196 | 110.2 | 0.841 | 0.716 | 0.841 |
| rs2844521 | 31139722 | 113.2 | 0.841 | 0.712 | 0.841 |
| rs3868075 | 30938573 | 113.2 | 0.84 | 0.789 | 0.84 |
| rs7766843 | 32456464 | 116.1 | 0.839 | 0.817 | 0.839 |
| rs4151651 | 31686372 | 110.2 | 0.839 | 0.804 | 0.839 |
| rs2394609 | 29460425 | 100.2 | 0.839 | 0.772 | 0.839 |
| rs2239805 | 32182133 | 103.2 | 0.839 | 0.678 | 0.839 |
| rs5007260 | 32398516 | 113.2 | 0.838 | 0.829 | 0.838 |
| rs2073044 | 32109743 | 98.2 | 0.838 | 0.474 | 0.838 |
| rs3765608 | 29746524 | 107.2 | 0.837 | 0.804 | 0.837 |
| rs769176 | 31606370 | 111.2 | 0.837 | 0.726 | 0.837 |
| rs7773694 | 32707900 | 116.1 | 0.836 | 0.823 | 0.836 |
| rs3128935 | 32973851 | 103.2 | 0.836 | 0.804 | 0.836 |
| rs2853950 | 31295483 | 100.2 | 0.835 | 0.736 | 0.835 |
| rs494620 | 31609471 | 113.2 | 0.835 | 0.603 | 0.835 |
| rs1633089 | 29778704 | 98.2 | 0.834 | 0.806 | 0.834 |
| rs2267642 | 30860223 | 100.2 | 0.834 | 0.806 | 0.834 |
| rs2259571 | 31642510 | 100.2 | 0.834 | 0.69 | 0.834 |
| rs1557608 | 30179784 | 113.2 | 0.834 | 0.667 | 0.834 |
| rs2530708 | 30712449 | 111.1 | 0.833 | 0.768 | 0.833 |
| rs7769930 | 30089601 | 116.1 | 0.833 | 0.408 | 0.833 |
| rs2097274 | 32613469 | 111.2 | 0.832 | 0.696 | 0.832 |
| rs422878 | 29593400 | 110.2 | 0.831 | 0.778 | 0.831 |
| rs6457643 | 32705984 | 116.1 | 0.831 | 0.675 | 0.831 |
| rs7381617 | 31547789 | 116.1 | 0.831 | 0.566 | 0.831 |
| rs3129270 | 33098870 | 103.2 | 0.83 | 0.626 | 0.83 |
| rs1632914 | 29658004 | 89.2 | 0.828 | 0.665 | 0.828 |
| rs3129808 | 30106312 | 103.2 | 0.828 | 0.565 | 0.828 |
| rs4713604 | 32973152 | 116.2 | 0.828 | 0.439 | 0.828 |
| rs241436 | 32568633 | 110.2 | 0.827 | 0.784 | 0.827 |
| rs3132555 | 31143716 | 113.2 | 0.827 | 0.437 | 0.827 |
| rs2517461 | 30957390 | 100.2 | 0.827 | 0.433 | 0.827 |
| rs5021448 | 32709272 | 113.2 | 0.827 | 0.347 | 0.827 |
| rs4122198 | 29516351 | 111.2 | 0.826 | 0.729 | 0.826 |
| rs2227134 | 32721678 | 96.2 | 0.826 | 0.511 | 0.826 |
| rs4713605 | 32979375 | 111.2 | 0.825 | 0.823 | 0.825 |
| rs1265158 | 30911499 | 100.2 | 0.825 | 0.769 | 0.825 |
| rs3135382 | 32402917 | 103.2 | 0.825 | 0.593 | 0.825 |
| rs3129701 | 30003430 | 103.2 | 0.824 | 0.8 | 0.824 |
| rs2244546 | 31494553 | 111.2 | 0.824 | 0.729 | 0.824 |
| rs2395175 | 32430808 | 113.2 | 0.823 | 0.815 | 0.823 |
| rs2844793 | 30141263 | 110.1 | 0.823 | 0.786 | 0.823 |
| rs2395327 | 33070364 | 116.2 | 0.823 | 0.77 | 0.823 |
| rs1265074 | 31177039 | 100.2 | 0.823 | 0.743 | 0.823 |
| rs3117228 | 33057882 | 103.2 | 0.823 | 0.676 | 0.823 |
| rs429479 | 29143078 | 113.2 | 0.823 | 0.608 | 0.823 |
| rs1634720 | 30747941 | 111.2 | 0.822 | 0.808 | 0.822 |
| rs3095302 | 30863824 | 103.2 | 0.822 | 0.616 | 0.822 |
| rs423023 | 32208035 | 113.2 | 0.821 | 0.786 | 0.821 |
| rs6456880 | 29025797 | 116.1 | 0.821 | 0.771 | 0.821 |
| rs1065461 | 31191308 | 113.2 | 0.821 | 0.736 | 0.821 |
| rs6923313 | 31300678 | 116.1 | 0.821 | 0.632 | 0.821 |
| rs1367730 | 33059561 | 103.2 | 0.821 | 0.61 | 0.821 |
| rs1800629 | 31601712 | 116.2 | 0.82 | 0.778 | 0.82 |
| rs2240070 | 30132297 | 98.2 | 0.82 | 0.756 | 0.82 |
| rs520803 | 32214630 | 96.1 | 0.82 | 0.716 | 0.82 |
| rs3734836 | 29750831 | 107.2 | 0.82 | 0.664 | 0.82 |
| rs3132468 | 31534222 | 103.1 | 0.819 | 0.662 | 0.819 |
| rs3094159 | 29901861 | 103.2 | 0.818 | 0.734 | 0.818 |
| rs3180665 | 31665730 | 113.1 | 0.818 | 0.668 | 0.818 |
| rs2518027 | 31495195 | 113.1 | 0.817 | 0.719 | 0.817 |
| rs1362068 | 29512866 | 110.2 | 0.817 | 0.448 | 0.817 |
| rs1264542 | 30447711 | 113.1 | 0.816 | 0.743 | 0.816 |
| rs2223638 | 32095398 | 113.2 | 0.816 | 0.322 | 0.816 |
| rs2281390 | 33061116 | 103.2 | 0.815 | 0.752 | 0.815 |
| rs1265094 | 31170680 | 100.2 | 0.815 | 0.726 | 0.815 |
| rs2523994 | 30131102 | 100.2 | 0.815 | 0.675 | 0.815 |
| rs2857129 | 32778159 | 113.2 | 0.815 | 0.605 | 0.815 |
| rs886403 | 30728376 | 110.1 | 0.814 | 0.809 | 0.814 |
| rs2213565 | 32707975 | 113.2 | 0.814 | 0.803 | 0.814 |
| rs1419658 | 29921685 | 108.2 | 0.814 | 0.794 | 0.814 |
| rs2022109 | 32899182 | 94.2 | 0.814 | 0.783 | 0.814 |
| rs3128928 | 33067600 | 103.2 | 0.814 | 0.737 | 0.814 |
| rs707913 | 31145590 | 111.2 | 0.814 | 0.643 | 0.814 |
| rs2027856 | 32428473 | 113.2 | 0.814 | 0.535 | 0.814 |
| rs2747454 | 29425871 | 100.2 | 0.813 | 0.777 | 0.813 |
| rs3132552 | 31146072 | 113.2 | 0.813 | 0.742 | 0.813 |
| rs3130989 | 31150143 | 110.2 | 0.813 | 0.517 | 0.813 |
| rs3817964 | 32138754 | 107.2 | 0.812 | 0.811 | 0.812 |
| rs154971 | 32903135 | 100.2 | 0.812 | 0.735 | 0.812 |
| rs4713326 | 30444714 | 111.2 | 0.812 | 0.678 | 0.812 |
| rs3132499 | 30978678 | 103.2 | 0.811 | 0.764 | 0.811 |
| rs2395318 | 33067683 | 103.2 | 0.81 | 0.79 | 0.81 |
| rs3823343 | 29683869 | 107.2 | 0.81 | 0.769 | 0.81 |
| rs2071463 | 32805941 | 110.2 | 0.81 | 0.747 | 0.81 |
| rs2857770 | 29405474 | 100.2 | 0.809 | 0.784 | 0.809 |
| rs4248154 | 30773374 | 113.2 | 0.808 | 0.797 | 0.808 |
| rs3132595 | 30671158 | 103.2 | 0.808 | 0.749 | 0.808 |
| rs3749970 | 29113580 | 107.2 | 0.807 | 0.803 | 0.807 |
| rs541862 | 31687709 | 110.1 | 0.807 | 0.772 | 0.807 |
| rs2075582 | 31556433 | 103.2 | 0.807 | 0.769 | 0.807 |
| rs2854048 | 31961875 | 110.1 | 0.807 | 0.769 | 0.807 |
| rs1557607 | 30179409 | 113.2 | 0.807 | 0.751 | 0.807 |
| rs1362070 | 29513057 | 110.2 | 0.807 | 0.708 | 0.807 |
| rs2621337 | 32778185 | 113.2 | 0.807 | 0.692 | 0.807 |
| rs3807030 | 30094479 | 107.2 | 0.806 | 0.798 | 0.806 |
| rs2596502 | 31379784 | 100.2 | 0.806 | 0.786 | 0.806 |
| rs2076530 | 32383302 | 98.2 | 0.806 | 0.238 | 0.806 |
| rs885932 | 29903534 | 111.2 | 0.805 | 0.79 | 0.805 |
| rs2517598 | 30141041 | 110.1 | 0.805 | 0.714 | 0.805 |
| rs2524079 | 31301478 | 100.2 | 0.805 | 0.677 | 0.805 |
| rs4286818 | 30843042 | 111.2 | 0.805 | 0.601 | 0.805 |
| rs4151650 | 31685648 | 110.2 | 0.805 | 0.552 | 0.805 |
| rs2523933 | 29703050 | 100.2 | 0.804 | 0.795 | 0.804 |
| rs194675 | 32899155 | 113.2 | 0.804 | 0.744 | 0.804 |
| rs3130615 | 31534317 | 103.1 | 0.804 | 0.688 | 0.804 |
| rs2050188 | 32365686 | 113.2 | 0.804 | 0.53 | 0.804 |
| rs2395173 | 32430641 | 113.2 | 0.804 | 0.144 | 0.804 |
| rs1573299 | 30177167 | 113.2 | 0.803 | 0.784 | 0.803 |
| rs417812 | 32975528 | 80.2 | 0.803 | 0.709 | 0.803 |
| rs1633029 | 29517063 | 89.2 | 0.803 | 0.694 | 0.803 |
| rs7454158 | 32779436 | 116.1 | 0.803 | 0.643 | 0.803 |
| rs2516670 | 30205757 | 100.2 | 0.803 | 0.585 | 0.803 |
| rs4151652 | 31686704 | 110.1 | 0.802 | 0.77 | 0.802 |
| rs5026741 | 32465623 | 113.2 | 0.802 | 0.707 | 0.802 |
| rs2158286 | 30101752 | 100.2 | 0.802 | 0.686 | 0.802 |
| rs1051790 | 31149714 | 110.2 | 0.802 | 0.667 | 0.802 |
| rs3828799 | 32630598 | 107.2 | 0.801 | 0.756 | 0.801 |
| rs1737005 | 29823614 | 89.2 | 0.801 | 0.681 | 0.801 |
| rs3132946 | 31960785 | 103.2 | 0.801 | 0.676 | 0.801 |
| rs3132467 | 31534547 | 103.2 | 0.801 | 0.664 | 0.801 |
| rs2076542 | 32087884 | 111.1 | 0.801 | 0.661 | 0.801 |
| rs3828882 | 31150378 | 107.2 | 0.8 | 0.766 | 0.8 |
| rs3094717 | 30446824 | 103.2 | 0.8 | 0.627 | 0.8 |
| rs3094214 | 31149148 | 113.2 | 0.8 | 0.604 | 0.8 |
| rs2734984 | 29591589 | 100.2 | 0.799 | 0.79 | 0.799 |
| rs3131018 | 30914340 | 110.2 | 0.799 | 0.762 | 0.799 |
| rs419957 | 29568207 | 100.2 | 0.799 | 0.728 | 0.799 |
| rs1737075 | 29497470 | 89.2 | 0.799 | 0.72 | 0.799 |
| rs3765607 | 29746598 | 107.2 | 0.799 | 0.65 | 0.799 |
| rs1800683 | 31598752 | 113.2 | 0.799 | 0.51 | 0.799 |
| rs3763315 | 32147411 | 107.2 | 0.798 | 0.791 | 0.798 |
| rs2187824 | 32458588 | 96.2 | 0.798 | 0.665 | 0.798 |
| rs6457041 | 29823581 | 116.1 | 0.798 | 0.586 | 0.798 |
| rs2285797 | 30180892 | 100.2 | 0.797 | 0.794 | 0.797 |
| rs2621336 | 32778512 | 100.2 | 0.797 | 0.792 | 0.797 |
| rs4713614 | 33129434 | 111.2 | 0.797 | 0.77 | 0.797 |
| rs4428528 | 32449831 | 113.2 | 0.797 | 0.746 | 0.797 |
| rs2071365 | 33034235 | 98.2 | 0.797 | 0.67 | 0.797 |
| rs2246638 | 30010265 | 100.2 | 0.797 | 0.627 | 0.797 |
| rs1264697 | 30128325 | 100.2 | 0.796 | 0.781 | 0.796 |
| rs2280773 | 31993248 | 100.1 | 0.796 | 0.757 | 0.796 |
| rs4248169 | 32723342 | 111.2 | 0.795 | 0.781 | 0.795 |
| rs1632941 | 29567443 | 100.2 | 0.795 | 0.761 | 0.795 |
| rs1610653 | 29831851 | 100.2 | 0.795 | 0.749 | 0.795 |
| rs6911894 | 29566450 | 116.1 | 0.795 | 0.739 | 0.795 |
| rs3778614 | 29823881 | 107.2 | 0.795 | 0.709 | 0.795 |
| rs2523619 | 31376893 | 113.2 | 0.795 | 0.7 | 0.795 |
| rs3213483 | 32721365 | 111.2 | 0.795 | 0.655 | 0.795 |
| rs2857713 | 31311314 | 113.2 | 0.794 | 0.736 | 0.794 |
| rs2072632 | 31692233 | 110.1 | 0.794 | 0.629 | 0.794 |
| rs2074469 | 29470395 | 98.2 | 0.793 | 0.786 | 0.793 |
| rs2071876 | 32719183 | 103.2 | 0.793 | 0.768 | 0.793 |
| rs3819686 | 31149590 | 107.2 | 0.793 | 0.758 | 0.793 |
| rs1049414 | 32719364 | 103.2 | 0.793 | 0.723 | 0.793 |
| rs1048412 | 30093660 | 110.2 | 0.793 | 0.721 | 0.793 |
| rs2269420 | 31930910 | 100.1 | 0.792 | 0.735 | 0.792 |
| rs7750641 | 31190117 | 116.1 | 0.792 | 0.718 | 0.792 |
| rs2647027 | 32636584 | 108.2 | 0.792 | 0.43 | 0.792 |
| rs3815095 | 30863965 | 107.2 | 0.791 | 0.78 | 0.791 |
| rs3130999 | 30861955 | 110.2 | 0.791 | 0.667 | 0.791 |
| rs3134940 | 32169562 | 103.1 | 0.791 | 0.168 | 0.791 |
| rs743400 | 31377869 | 86.2 | 0.79 | 0.658 | 0.79 |
| rs4711187 | 29138345 | 111.2 | 0.79 | 0.641 | 0.79 |
| rs2395038 | 31231209 | 103.2 | 0.79 | 0.547 | 0.79 |
| rs2239689 | 31801041 | 98.2 | 0.789 | 0.754 | 0.789 |
| rs995185 | 29591593 | 110.2 | 0.789 | 0.666 | 0.789 |
| rs3830041 | 31962096 | 107.1 | 0.788 | 0.717 | 0.788 |
| rs2523945 | 30002707 | 110.2 | 0.788 | 0.677 | 0.788 |
| rs3130099 | 33283628 | 103.1 | 0.788 | 0.602 | 0.788 |
| rs3130374 | 30092094 | 103.2 | 0.787 | 0.644 | 0.787 |
| rs2524272 | 29453158 | 100.2 | 0.786 | 0.783 | 0.786 |
| rs3093558 | 31606500 | 103.2 | 0.786 | 0.694 | 0.786 |
| rs7656 | 32398715 | 108.2 | 0.786 | 0.619 | 0.786 |
| rs3093992 | 31549874 | 103.2 | 0.786 | 0.484 | 0.786 |
| rs2428509 | 29590571 | 100.1 | 0.785 | 0.673 | 0.785 |
| rs2071292 | 31926705 | 98.1 | 0.785 | 0.601 | 0.785 |
| rs107822 | 33177022 | 108.1 | 0.785 | 0.513 | 0.785 |
| rs3734860 | 31161268 | 107.2 | 0.785 | 0.254 | 0.785 |
| rs4151667 | 31972775 | 110.2 | 0.784 | 0.602 | 0.784 |
| rs887466 | 30914269 | 110.2 | 0.784 | 0.55 | 0.784 |
| rs2857096 | 32912512 | 100.2 | 0.784 | 0.404 | 0.784 |
| rs3130976 | 31142746 | 110.2 | 0.783 | 0.599 | 0.783 |
| rs6910473 | 31201636 | 116.1 | 0.782 | 0.673 | 0.782 |
| rs1633012 | 29821245 | 113.2 | 0.782 | 0.627 | 0.782 |
| rs2523399 | 29470837 | 100.2 | 0.781 | 0.773 | 0.781 |
| rs2071786 | 28974989 | 96.2 | 0.781 | 0.268 | 0.781 |
| rs3823359 | 30002676 | 107.2 | 0.78 | 0.714 | 0.78 |
| rs400488 | 30003417 | 100.2 | 0.78 | 0.696 | 0.78 |
| rs2239697 | 32553654 | 98.2 | 0.78 | 0.66 | 0.78 |
| rs4959041 | 30139153 | 111.2 | 0.78 | 0.592 | 0.78 |
| rs1042434 | 33029570 | 116.2 | 0.78 | 0.563 | 0.78 |
| rs3819275 | 31149472 | 107.2 | 0.78 | 0.538 | 0.78 |
| rs381218 | 32970802 | 80.2 | 0.779 | 0.762 | 0.779 |
| rs29230 | 29638845 | 98.2 | 0.779 | 0.738 | 0.779 |
| rs2301226 | 33036043 | 116.2 | 0.778 | 0.771 | 0.778 |
| rs742870 | 33077715 | 111.2 | 0.778 | 0.758 | 0.778 |
| rs6905862 | 31188444 | 116.1 | 0.777 | 0.741 | 0.777 |
| rs3129938 | 32107252 | 103.2 | 0.777 | 0.691 | 0.777 |
| rs1736939 | 29564143 | 101.2 | 0.777 | 0.47 | 0.777 |
| rs4548006 | 32719166 | 113.2 | 0.777 | 0.461 | 0.777 |
| rs2857767 | 29405026 | 100.2 | 0.776 | 0.704 | 0.776 |
| rs241442 | 32567925 | 110.2 | 0.776 | 0.508 | 0.776 |
| rs3093671 | 31605661 | 111.2 | 0.775 | 0.712 | 0.775 |
| rs2857433 | 30181405 | 100.2 | 0.775 | 0.646 | 0.775 |
| rs1800453 | 32808388 | 96.2 | 0.775 | 0.539 | 0.775 |
| rs1049628 | 30927919 | 108.2 | 0.774 | 0.724 | 0.774 |
| rs2769 | 31380634 | 101.2 | 0.773 | 0.568 | 0.773 |
| rs3763295 | 31646775 | 107.2 | 0.772 | 0.765 | 0.772 |
| rs3763338 | 28956863 | 116.2 | 0.772 | 0.761 | 0.772 |
| rs6910733 | 29761908 | 116.1 | 0.772 | 0.712 | 0.772 |
| rs3830037 | 31962377 | 107.1 | 0.771 | 0.649 | 0.771 |
| rs2858330 | 32429472 | 100.2 | 0.77 | 0.494 | 0.77 |
| rs1076711 | 32071131 | 86.2 | 0.77 | 0.438 | 0.77 |
| rs2272874 | 29467003 | 100.2 | 0.769 | 0.768 | 0.769 |
| rs762324 | 30516617 | 111.2 | 0.769 | 0.752 | 0.769 |
| rs2061952 | 32779129 | 111.2 | 0.769 | 0.731 | 0.769 |
| rs4647195 | 31311715 | 111.2 | 0.769 | 0.691 | 0.769 |
| rs2523400 | 29470542 | 100.2 | 0.769 | 0.643 | 0.769 |
| rs1041981 | 31311542 | 111.2 | 0.769 | 0.616 | 0.769 |
| rs1367729 | 33059715 | 103.2 | 0.769 | 0.445 | 0.769 |
| rs2523943 | 30003309 | 100.2 | 0.768 | 0.661 | 0.768 |
| rs1233421 | 29563952 | 100.2 | 0.768 | 0.624 | 0.768 |
| rs4988888 | 32629726 | 116.2 | 0.768 | 0.544 | 0.768 |
| rs4713260 | 29666407 | 111.1 | 0.767 | 0.743 | 0.767 |
| rs3095227 | 31549743 | 103.2 | 0.767 | 0.71 | 0.767 |
| rs2596538 | 31139390 | 110.2 | 0.767 | 0.624 | 0.767 |
| rs1480382 | 32735686 | 88.2 | 0.767 | 0.519 | 0.767 |
| rs2229699 | 31607537 | 98.2 | 0.767 | 0.505 | 0.767 |
| rs2276447 | 31384819 | 100.2 | 0.766 | 0.761 | 0.766 |
| rs2074511 | 30950188 | 111.2 | 0.766 | 0.702 | 0.766 |
| rs2285795 | 30182114 | 100.2 | 0.766 | 0.625 | 0.766 |
| rs2523959 | 29710736 | 110.2 | 0.766 | 0.624 | 0.766 |
| rs5020983 | 30000760 | 113.2 | 0.766 | 0.624 | 0.766 |
| rs2071353 | 33037322 | 98.2 | 0.766 | 0.559 | 0.766 |
| rs1610646 | 29823338 | 98.2 | 0.765 | 0.759 | 0.765 |
| rs3117035 | 33079243 | 103.2 | 0.765 | 0.691 | 0.765 |
| rs2071470 | 32778168 | 100.2 | 0.765 | 0.663 | 0.765 |
| rs376892 | 32974334 | 80.2 | 0.765 | 0.631 | 0.765 |
| rs1631950 | 29566179 | 89.2 | 0.765 | 0.466 | 0.765 |
| rs206781 | 32716890 | 106.2 | 0.764 | 0.763 | 0.764 |
| rs2071592 | 31574059 | 103.2 | 0.764 | 0.641 | 0.764 |
| rs4713274 | 29708251 | 111.2 | 0.764 | 0.628 | 0.764 |
| rs4959119 | 32616486 | 111.2 | 0.764 | 0.438 | 0.764 |
| rs3129744 | 32458051 | 103.1 | 0.763 | 0.522 | 0.763 |
| rs1737086 | 29779654 | 110.2 | 0.762 | 0.715 | 0.762 |
| rs2523960 | 29710710 | 110.2 | 0.762 | 0.708 | 0.762 |
| rs5020984 | 30000734 | 113.2 | 0.762 | 0.708 | 0.762 |
| rs3093542 | 31311451 | 103.2 | 0.762 | 0.62 | 0.762 |
| rs2127680 | 32811444 | 113.2 | 0.761 | 0.744 | 0.761 |
| rs2844508 | 31495218 | 113.1 | 0.761 | 0.729 | 0.761 |
| rs3093554 | 31608088 | 103.2 | 0.761 | 0.701 | 0.761 |
| rs1268928 | 31139705 | 110.2 | 0.761 | 0.697 | 0.761 |
| rs2294881 | 32138361 | 111.2 | 0.761 | 0.664 | 0.761 |
| rs7773149 | 32707608 | 116.1 | 0.76 | 0.745 | 0.76 |
| rs2524197 | 30192375 | 111.2 | 0.76 | 0.593 | 0.76 |
| rs1419635 | 29423916 | 113.2 | 0.76 | 0.535 | 0.76 |
| rs7755774 | 32470680 | 116.1 | 0.759 | 0.702 | 0.759 |
| rs3131001 | 30862289 | 110.2 | 0.758 | 0.568 | 0.758 |
| rs1610676 | 29853952 | 89.2 | 0.757 | 0.739 | 0.757 |
| rs6901686 | 29778245 | 116.1 | 0.757 | 0.682 | 0.757 |
| rs3094200 | 30863890 | 103.2 | 0.756 | 0.741 | 0.756 |
| rs2523705 | 31222438 | 100.2 | 0.756 | 0.696 | 0.756 |
| rs885948 | 31201360 | 113.2 | 0.756 | 0.634 | 0.756 |
| rs2009658 | 31309002 | 103.2 | 0.755 | 0.683 | 0.755 |
| rs2517754 | 29667438 | 111.2 | 0.755 | 0.614 | 0.755 |
| rs2596499 | 31380181 | 100.2 | 0.755 | 0.518 | 0.755 |
| rs929156 | 29910457 | 111.2 | 0.755 | 0.419 | 0.755 |
| rs2076312 | 33134883 | 100.2 | 0.754 | 0.554 | 0.754 |
| rs3093666 | 31604414 | 103.1 | 0.753 | 0.736 | 0.753 |
| rs3132520 | 31203843 | 103.2 | 0.753 | 0.701 | 0.753 |
| rs7773175 | 31300267 | 116.1 | 0.753 | 0.628 | 0.753 |
| rs3116956 | 33130518 | 103.2 | 0.753 | 0.557 | 0.753 |
| rs3823417 | 31161695 | 107.2 | 0.752 | 0.644 | 0.752 |
| rs1265092 | 31168144 | 100.2 | 0.751 | 0.528 | 0.751 |
| rs204889 | 31856849 | 87.2 | 0.751 | 0.495 | 0.751 |
| rs910320 | 33068431 | 103.2 | 0.75 | 0.746 | 0.75 |
| rs2239886 | 30441458 | 98.2 | 0.75 | 0.724 | 0.75 |
| rs2158279 | 29428702 | 113.2 | 0.75 | 0.705 | 0.75 |
| rs463260 | 33249683 | 87.1 | 0.749 | 0.708 | 0.749 |
| rs2523713 | 30234061 | 110.2 | 0.749 | 0.441 | 0.749 |
| rs2072915 | 33163529 | 116.2 | 0.748 | 0.712 | 0.748 |
| rs2187578 | 31376229 | 113.2 | 0.748 | 0.347 | 0.748 |
| rs2246626 | 31537860 | 100.2 | 0.747 | 0.736 | 0.747 |
| rs2517672 | 29708020 | 110.2 | 0.747 | 0.334 | 0.747 |
| rs2076489 | 29638889 | 98.2 | 0.746 | 0.738 | 0.746 |
| rs1264322 | 30628652 | 100.2 | 0.745 | 0.727 | 0.745 |
| rs2523970 | 29709230 | 100.2 | 0.744 | 0.737 | 0.744 |
| rs2073722 | 31190449 | 98.2 | 0.744 | 0.599 | 0.744 |
| rs6915224 | 32487588 | 116.1 | 0.744 | 0.51 | 0.744 |
| rs209130 | 28930350 | 96.2 | 0.743 | 0.686 | 0.743 |
| rs5017092 | 32719034 | 113.2 | 0.743 | 0.578 | 0.743 |
| rs3130552 | 31142933 | 110.2 | 0.743 | 0.511 | 0.743 |
| rs2247720 | 29592160 | 100.2 | 0.742 | 0.69 | 0.742 |
| rs3807036 | 30105703 | 107.2 | 0.742 | 0.367 | 0.742 |
| rs1049622 | 30629615 | 102.2 | 0.741 | 0.66 | 0.741 |
| rs2179915 | 33067181 | 96.2 | 0.741 | 0.456 | 0.741 |
| rs6930217 | 29566220 | 116.1 | 0.74 | 0.7 | 0.74 |
| rs3093553 | 31608237 | 111.1 | 0.74 | 0.504 | 0.74 |
| rs6457597 | 32470203 | 116.1 | 0.74 | 0.44 | 0.74 |
| rs241437 | 32568441 | 110.2 | 0.739 | 0.641 | 0.739 |
| rs5026740 | 32465511 | 113.2 | 0.739 | 0.632 | 0.739 |
| rs15604 | 33260414 | 116.1 | 0.739 | 0.548 | 0.739 |
| rs2071800 | 32484900 | 98.2 | 0.738 | 0.732 | 0.738 |
| rs2844522 | 31139458 | 111.2 | 0.738 | 0.722 | 0.738 |
| rs2516409 | 31522143 | 111.2 | 0.738 | 0.657 | 0.738 |
| rs7770592 | 29458172 | 116.1 | 0.737 | 0.651 | 0.737 |
| rs444013 | 29294404 | 111.2 | 0.736 | 0.735 | 0.736 |
| rs2844846 | 29777275 | 113.2 | 0.736 | 0.701 | 0.736 |
| rs3757346 | 30860895 | 107.2 | 0.736 | 0.692 | 0.736 |
| rs3134929 | 32211848 | 111.1 | 0.736 | 0.689 | 0.736 |
| rs401775 | 31701895 | 110.1 | 0.736 | 0.638 | 0.736 |
| rs6924267 | 32468059 | 116.1 | 0.736 | 0.413 | 0.736 |
| rs2517673 | 29708000 | 110.2 | 0.736 | 0.33 | 0.736 |
| rs1736980 | 29834450 | 89.2 | 0.735 | 0.677 | 0.735 |
| rs1611203 | 29824925 | 89.2 | 0.734 | 0.594 | 0.734 |
| rs7768484 | 29916363 | 116.1 | 0.733 | 0.713 | 0.733 |
| rs7741100 | 29830694 | 116.1 | 0.733 | 0.636 | 0.733 |
| rs2294884 | 32138016 | 100.2 | 0.733 | 0.612 | 0.733 |
| rs2308935 | 33029500 | 100.2 | 0.733 | 0.594 | 0.733 |
| rs2282850 | 33268551 | 100.2 | 0.732 | 0.666 | 0.732 |
| rs2073154 | 29427252 | 98.2 | 0.732 | 0.654 | 0.732 |
| rs1265095 | 31170430 | 100.2 | 0.731 | 0.661 | 0.731 |
| rs3095317 | 30859429 | 103.2 | 0.731 | 0.555 | 0.731 |
| rs3778630 | 30862086 | 107.2 | 0.731 | 0.421 | 0.731 |
| rs3869090 | 30648016 | 111.1 | 0.73 | 0.644 | 0.73 |
| rs415929 | 32215059 | 113.2 | 0.73 | 0.566 | 0.73 |
| rs2747431 | 29711081 | 100.2 | 0.729 | 0.671 | 0.729 |
| rs2428507 | 30000064 | 101.2 | 0.729 | 0.549 | 0.729 |
| rs2295666 | 31689094 | 100.2 | 0.728 | 0.727 | 0.728 |
| rs1233427 | 29564521 | 100.2 | 0.728 | 0.645 | 0.728 |
| rs7774644 | 32725643 | 116.1 | 0.728 | 0.597 | 0.728 |
| rs3095309 | 31152278 | 110.1 | 0.726 | 0.5 | 0.726 |
| rs5872 | 31696571 | 106.1 | 0.725 | 0.71 | 0.725 |
| rs1033500 | 32333512 | 113.2 | 0.725 | 0.649 | 0.725 |
| rs3763306 | 32140400 | 111.2 | 0.724 | 0.522 | 0.724 |
| rs635688 | 32936576 | 110.1 | 0.724 | 0.44 | 0.724 |
| rs4151669 | 31685902 | 110.2 | 0.721 | 0.597 | 0.721 |
| rs2076527 | 32383700 | 98.2 | 0.72 | 0.404 | 0.72 |
| rs3178162 | 30941911 | 106.1 | 0.719 | 0.643 | 0.719 |
| rs241449 | 32567410 | 110.2 | 0.719 | 0.533 | 0.719 |
| rs4435981 | 32975065 | 111.2 | 0.717 | 0.67 | 0.717 |
| rs6456897 | 29103831 | 116.1 | 0.716 | 0.684 | 0.716 |
| rs3828923 | 29658590 | 107.2 | 0.716 | 0.682 | 0.716 |
| rs2179920 | 33060321 | 103.2 | 0.716 | 0.616 | 0.716 |
| rs3218806 | 30646422 | 106.2 | 0.716 | 0.574 | 0.716 |
| rs2076175 | 29778609 | 98.2 | 0.716 | 0.542 | 0.716 |
| rs3871246 | 30916189 | 113.2 | 0.715 | 0.594 | 0.715 |
| rs2281388 | 33061565 | 100.2 | 0.714 | 0.628 | 0.714 |
| rs3763314 | 32147375 | 107.2 | 0.714 | 0.269 | 0.714 |
| rs6913173 | 32636337 | 116.1 | 0.713 | 0.698 | 0.713 |
| rs3873408 | 31496591 | 113.2 | 0.711 | 0.629 | 0.711 |
| rs3997995 | 31380339 | 113.2 | 0.709 | 0.57 | 0.709 |
| rs2523924 | 30745982 | 100.2 | 0.708 | 0.611 | 0.708 |
| rs2239517 | 30925928 | 113.1 | 0.708 | 0.359 | 0.708 |
| rs3094225 | 31173872 | 103.2 | 0.707 | 0.696 | 0.707 |
| rs3869117 | 30976681 | 113.2 | 0.707 | 0.63 | 0.707 |
| rs3734837 | 30141233 | 107.1 | 0.707 | 0.519 | 0.707 |
| rs2524180 | 30196888 | 100.2 | 0.705 | 0.604 | 0.705 |
| rs2844804 | 30002509 | 110.2 | 0.704 | 0.537 | 0.704 |
| rs2206618 | 32095301 | 113.2 | 0.704 | 0.508 | 0.704 |
| rs406424 | 29709781 | 113.2 | 0.703 | 0.679 | 0.703 |
| rs3830038 | 31962338 | 107.1 | 0.703 | 0.444 | 0.703 |
| rs1048709 | 31685693 | 110.2 | 0.701 | 0.595 | 0.701 |
| rs3093661 | 31602439 | 103.2 | 0.701 | 0.553 | 0.701 |
| rs2252856 | 30954243 | 100.1 | 0.701 | 0.52 | 0.701 |
| rs2857177 | 32523597 | 100.2 | 0.7 | 0.662 | 0.7 |
| rs9968 | 29563475 | 106.2 | 0.698 | 0.669 | 0.698 |
| rs365052 | 29710915 | 80.2 | 0.698 | 0.455 | 0.698 |
| rs1800624 | 31923144 | 96.1 | 0.697 | 0.624 | 0.697 |
| rs2856715 | 32621706 | 100.2 | 0.697 | 0.47 | 0.697 |
| rs3130444 | 31290938 | 103.2 | 0.696 | 0.669 | 0.696 |
| rs2071551 | 32731026 | 100.2 | 0.696 | 0.532 | 0.696 |
| rs2301221 | 33031662 | 100.2 | 0.696 | 0.446 | 0.696 |
| rs6927322 | 31149785 | 116.1 | 0.696 | 0.376 | 0.696 |
| rs7768179 | 30034379 | 116.1 | 0.695 | 0.657 | 0.695 |
| rs6913646 | 32724397 | 116.1 | 0.695 | 0.588 | 0.695 |
| rs2256820 | 29710864 | 110.2 | 0.693 | 0.68 | 0.693 |
| rs4713410 | 30753459 | 111.2 | 0.692 | 0.652 | 0.692 |
| rs872956 | 33069078 | 103.2 | 0.692 | 0.475 | 0.692 |
| rs4959077 | 31534364 | 111.2 | 0.691 | 0.671 | 0.691 |
| rs2071282 | 32214970 | 98.2 | 0.691 | 0.628 | 0.691 |
| rs1264328 | 30620900 | 100.2 | 0.691 | 0.614 | 0.691 |
| rs3918149 | 32707130 | 108.2 | 0.69 | 0.368 | 0.69 |
| rs2157051 | 32429381 | 103.2 | 0.69 | 0.156 | 0.69 |
| rs7750514 | 30034401 | 116.1 | 0.689 | 0.594 | 0.689 |
| rs3128964 | 33057265 | 103.2 | 0.689 | 0.459 | 0.689 |
| rs6912877 | 30011455 | 116.1 | 0.688 | 0.664 | 0.688 |
| rs1541268 | 30164163 | 113.2 | 0.687 | 0.259 | 0.687 |
| rs6906175 | 31429861 | 116.1 | 0.683 | 0.299 | 0.683 |
| rs2074506 | 30951282 | 113.2 | 0.681 | 0.612 | 0.681 |
| rs2239523 | 30860265 | 103.2 | 0.68 | 0.676 | 0.68 |
| rs2242665 | 31610067 | 103.2 | 0.68 | 0.662 | 0.68 |
| rs3131300 | 31922691 | 103.2 | 0.679 | 0.654 | 0.679 |
| rs465223 | 33361168 | 111.2 | 0.679 | 0.559 | 0.679 |
| rs3819299 | 31384121 | 107.2 | 0.676 | 0.613 | 0.676 |
| rs2844524 | 31139196 | 100.2 | 0.676 | 0.506 | 0.676 |
| rs6915440 | 32221961 | 116.1 | 0.675 | 0.623 | 0.675 |
| rs1614309 | 29516642 | 89.2 | 0.674 | 0.472 | 0.674 |
| rs2076183 | 29462061 | 98.2 | 0.673 | 0.66 | 0.673 |
| rs3763311 | 32146933 | 107.2 | 0.673 | 0.586 | 0.673 |
| rs3097650 | 33058887 | 103.2 | 0.673 | 0.167 | 0.673 |
| rs2743945 | 29513015 | 111.2 | 0.672 | 0.664 | 0.672 |
| rs1737087 | 29779007 | 111.2 | 0.672 | 0.42 | 0.672 |
| rs1611165 | 29854685 | 100.2 | 0.67 | 0.378 | 0.67 |
| rs3828913 | 31236553 | 107.2 | 0.67 | 0.213 | 0.67 |
| rs3763312 | 32147105 | 107.2 | 0.669 | 0.551 | 0.669 |
| rs2269711 | 30912370 | 103.2 | 0.669 | 0.51 | 0.669 |
| rs2243873 | 31922252 | 111.1 | 0.669 | 0.475 | 0.669 |
| rs2072108 | 31170818 | 98.2 | 0.668 | 0.624 | 0.668 |
| rs7770882 | 29428082 | 116.1 | 0.668 | 0.371 | 0.668 |
| rs1736935 | 29565201 | 110.2 | 0.667 | 0.293 | 0.667 |
| rs3823420 | 31161879 | 107.2 | 0.666 | 0.621 | 0.666 |
| rs2071571 | 32934044 | 98.2 | 0.666 | 0.455 | 0.666 |
| rs3093726 | 31605470 | 111.1 | 0.666 | 0.416 | 0.666 |
| rs973233 | 33132731 | 108.2 | 0.664 | 0.636 | 0.664 |
| rs1150772 | 30445385 | 100.2 | 0.664 | 0.564 | 0.664 |
| rs1264301 | 30943589 | 100.2 | 0.664 | 0.496 | 0.664 |
| rs367398 | 32217754 | 92.1 | 0.663 | 0.601 | 0.663 |
| rs241451 | 32567237 | 110.2 | 0.663 | 0.521 | 0.663 |
| rs2076528 | 32383680 | 98.2 | 0.662 | 0.407 | 0.662 |
| rs3115615 | 29910358 | 103.2 | 0.662 | 0.388 | 0.662 |
| rs2228088 | 31602286 | 98.2 | 0.66 | 0.593 | 0.66 |
| rs2269611 | 33285335 | 100.2 | 0.66 | 0.475 | 0.66 |
| rs408036 | 32975715 | 100.2 | 0.659 | 0.494 | 0.659 |
| rs3130998 | 30861703 | 110.2 | 0.657 | 0.515 | 0.657 |
| rs2022537 | 32072667 | 113.2 | 0.656 | 0.632 | 0.656 |
| rs2239520 | 31149724 | 103.2 | 0.656 | 0.468 | 0.656 |
| rs4993139 | 32629579 | 116.2 | 0.656 | 0.339 | 0.656 |
| rs3094215 | 31145487 | 111.2 | 0.655 | 0.619 | 0.655 |
| rs1063379 | 32716791 | 86.2 | 0.654 | 0.575 | 0.654 |
| rs259942 | 29785925 | 86.2 | 0.654 | 0.563 | 0.654 |
| rs3734861 | 31161286 | 107.2 | 0.654 | 0.302 | 0.654 |
| rs2499 | 29684300 | 108.2 | 0.653 | 0.595 | 0.653 |
| rs4713250 | 29866097 | 111.2 | 0.652 | 0.132 | 0.652 |
| rs2269426 | 31847256 | 100.2 | 0.651 | 0.529 | 0.651 |
| rs887467 | 30912422 | 100.2 | 0.649 | 0.636 | 0.649 |
| rs2269712 | 30911967 | 103.2 | 0.648 | 0.577 | 0.648 |
| rs2302396 | 31152095 | 103.2 | 0.645 | 0.551 | 0.645 |
| rs2008560 | 32724522 | 111.2 | 0.644 | 0.374 | 0.644 |
| rs1480383 | 32735647 | 88.2 | 0.643 | 0.451 | 0.643 |
| rs3129997 | 30718284 | 103.2 | 0.642 | 0.391 | 0.642 |
| rs2517588 | 29975472 | 111.2 | 0.639 | 0.441 | 0.639 |
| rs465474 | 33360594 | 98.2 | 0.637 | 0.626 | 0.637 |
| rs3218812 | 30938568 | 113.1 | 0.636 | 0.535 | 0.636 |
| rs2523791 | 29866076 | 100.2 | 0.633 | 0.124 | 0.633 |
| rs3828912 | 31236484 | 107.2 | 0.632 | 0.47 | 0.632 |
| rs2517444 | 30712534 | 111.1 | 0.632 | 0.145 | 0.632 |
| rs2257347 | 30953046 | 110.1 | 0.631 | 0.605 | 0.631 |
| rs364950 | 32977343 | 80.2 | 0.63 | 0.557 | 0.63 |
| rs2006164 | 32723777 | 111.2 | 0.63 | 0.544 | 0.63 |
| rs2857172 | 32525780 | 100.2 | 0.629 | 0.611 | 0.629 |
| rs3873243 | 29830196 | 111.2 | 0.629 | 0.327 | 0.629 |
| rs3763327 | 32184587 | 107.2 | 0.628 | 0.599 | 0.628 |
| rs1061801 | 33283785 | 106.2 | 0.627 | 0.601 | 0.627 |
| rs4713519 | 32033020 | 111.2 | 0.627 | 0.418 | 0.627 |
| rs909722 | 29833296 | 100.2 | 0.625 | 0.355 | 0.625 |
| rs2213569 | 32723140 | 113.2 | 0.621 | 0.617 | 0.621 |
| rs644827 | 31609199 | 110.2 | 0.62 | 0.608 | 0.62 |
| rs3763341 | 32972402 | 107.2 | 0.618 | 0.502 | 0.618 |
| rs2254287 | 33136669 | 100.2 | 0.617 | 0.614 | 0.617 |
| rs769178 | 31606195 | 111.2 | 0.617 | 0.587 | 0.617 |
| rs2270190 | 31141392 | 100.2 | 0.615 | 0.541 | 0.615 |
| rs986521 | 33128867 | 100.2 | 0.615 | 0.533 | 0.615 |
| rs206775 | 32725391 | 87.2 | 0.611 | 0.243 | 0.611 |
| rs241447 | 32567508 | 110.2 | 0.61 | 0.604 | 0.61 |
| rs2157336 | 32156818 | 103.2 | 0.61 | 0.49 | 0.61 |
| rs1044507 | 32189540 | 98.1 | 0.608 | 0.582 | 0.608 |
| rs3094204 | 30862750 | 110.2 | 0.608 | 0.566 | 0.608 |
| rs1611613 | 29916688 | 113.2 | 0.608 | 0.232 | 0.608 |
| rs1736927 | 29566873 | 108.2 | 0.607 | 0.598 | 0.607 |
| rs2855438 | 33131561 | 100.1 | 0.607 | 0.551 | 0.607 |
| rs3819284 | 31384521 | 107.2 | 0.607 | 0.502 | 0.607 |
| rs6913471 | 32365714 | 116.1 | 0.605 | 0.468 | 0.605 |
| rs969892 | 32087069 | 113.2 | 0.605 | 0.327 | 0.605 |
| rs2516507 | 31221971 | 100.2 | 0.602 | 0.462 | 0.602 |
| rs2071552 | 32807983 | 110.1 | 0.602 | 0.368 | 0.602 |
| rs375256 | 32977316 | 100.2 | 0.601 | 0.367 | 0.601 |
| rs2074465 | 29470901 | 98.2 | 0.599 | 0.572 | 0.599 |
| rs3130712 | 30980268 | 103.2 | 0.599 | 0.455 | 0.599 |
| rs2286654 | 30670992 | 111.2 | 0.596 | 0.577 | 0.596 |
| rs1265089 | 31169428 | 100.2 | 0.596 | 0.31 | 0.596 |
| rs915652 | 31519900 | 100.2 | 0.595 | 0.495 | 0.595 |
| rs362513 | 29299076 | 111.2 | 0.595 | 0.44 | 0.595 |
| rs3736710 | 33215306 | 107.2 | 0.594 | 0.516 | 0.594 |
| rs3130662 | 30774530 | 103.2 | 0.594 | 0.472 | 0.594 |
| rs2524056 | 31311508 | 113.2 | 0.594 | 0.331 | 0.594 |
| rs2395039 | 31231276 | 103.2 | 0.593 | 0.422 | 0.593 |
| rs683208 | 32708658 | 113.2 | 0.592 | 0.364 | 0.592 |
| rs1264320 | 30631758 | 106.2 | 0.591 | 0.566 | 0.591 |
| rs2074492 | 31302189 | 96.2 | 0.591 | 0.537 | 0.591 |
| rs4524656 | 32613832 | 113.2 | 0.591 | 0.532 | 0.591 |
| rs3130934 | 31189394 | 103.2 | 0.591 | 0.476 | 0.591 |
| rs1264457 | 30518794 | 100.2 | 0.591 | 0.422 | 0.591 |
| rs2242667 | 31554285 | 100.1 | 0.591 | 0.388 | 0.591 |
| rs7739536 | 29585928 | 116.1 | 0.589 | 0.47 | 0.589 |
| rs409715 | 32221751 | 80.2 | 0.589 | 0.395 | 0.589 |
| rs3828914 | 31236577 | 107.2 | 0.589 | 0.21 | 0.589 |
| rs2272991 | 29298357 | 102.2 | 0.587 | 0.49 | 0.587 |
| rs1362061 | 29141920 | 113.2 | 0.587 | 0.398 | 0.587 |
| rs3132377 | 28958067 | 103.1 | 0.587 | 0.377 | 0.587 |
| rs743771 | 32970291 | 113.2 | 0.586 | 0.521 | 0.586 |
| rs2523612 | 31379875 | 100.2 | 0.585 | 0.37 | 0.585 |
| rs4713220 | 29138253 | 111.2 | 0.583 | 0.58 | 0.583 |
| rs2301224 | 33031434 | 100.2 | 0.583 | 0.487 | 0.583 |
| rs3106191 | 33273203 | 103.2 | 0.581 | 0.267 | 0.581 |
| rs3749982 | 32633364 | 107.2 | 0.58 | 0.499 | 0.58 |
| rs1610678 | 29854145 | 89.2 | 0.58 | 0.117 | 0.58 |
| rs2524055 | 31311529 | 113.2 | 0.578 | 0.322 | 0.578 |
| rs2073721 | 31190423 | 98.2 | 0.577 | 0.414 | 0.577 |
| rs3132496 | 30979368 | 103.2 | 0.577 | 0.121 | 0.577 |
| rs2071653 | 29406686 | 103.2 | 0.575 | 0.567 | 0.575 |
| rs7772982 | 29403593 | 116.1 | 0.575 | 0.554 | 0.575 |
| rs2524094 | 31299349 | 100.2 | 0.575 | 0.469 | 0.575 |
| rs2252863 | 30954227 | 100.1 | 0.573 | 0.508 | 0.573 |
| rs2532939 | 30954218 | 100.1 | 0.572 | 0.571 | 0.572 |
| rs3129793 | 29038040 | 103.2 | 0.571 | 0.31 | 0.571 |
| rs3130926 | 31522591 | 110.2 | 0.57 | 0.368 | 0.57 |
| rs707918 | 31425490 | 113.1 | 0.57 | 0.304 | 0.57 |
| rs6925178 | 29035764 | 116.1 | 0.566 | 0.384 | 0.566 |
| rs6456902 | 29119757 | 116.1 | 0.566 | 0.192 | 0.566 |
| rs1610682 | 29863895 | 89.2 | 0.565 | 0.508 | 0.565 |
| rs2073720 | 31173643 | 98.2 | 0.565 | 0.495 | 0.565 |
| rs2071785 | 28975010 | 96.2 | 0.563 | 0.369 | 0.563 |
| rs2856993 | 32562160 | 106.2 | 0.563 | 0.271 | 0.563 |
| rs4959012 | 30175617 | 111.2 | 0.562 | 0.544 | 0.562 |
| rs614549 | 31611383 | 83.2 | 0.562 | 0.496 | 0.562 |
| rs3749963 | 32056183 | 107.1 | 0.562 | 0.443 | 0.562 |
| rs3757324 | 29459885 | 107.2 | 0.562 | 0.379 | 0.562 |
| rs6457150 | 30129367 | 116.1 | 0.562 | 0.356 | 0.562 |
| rs915651 | 31519942 | 100.2 | 0.561 | 0.504 | 0.561 |
| rs3130456 | 31187094 | 103.2 | 0.561 | 0.435 | 0.561 |
| rs1150739 | 30092134 | 100.2 | 0.559 | 0.264 | 0.559 |
| rs1611617 | 29919351 | 100.2 | 0.558 | 0.46 | 0.558 |
| rs3094139 | 29968753 | 103.2 | 0.558 | 0.232 | 0.558 |
| rs2394414 | 30843060 | 113.2 | 0.558 | 0.166 | 0.558 |
| rs11724 | 29292077 | 100.2 | 0.556 | 0.515 | 0.556 |
| rs1611185 | 29830915 | 113.2 | 0.556 | 0.499 | 0.556 |
| rs6940440 | 32454827 | 116.1 | 0.556 | 0.422 | 0.556 |
| rs7452208 | 31237066 | 116.1 | 0.555 | 0.256 | 0.555 |
| rs3763342 | 32972379 | 116.2 | 0.554 | 0.517 | 0.554 |
| rs2518030 | 31201840 | 100.2 | 0.553 | 0.479 | 0.553 |
| rs7772174 | 31387544 | 116.1 | 0.553 | 0.433 | 0.553 |
| rs3869097 | 30755228 | 113.2 | 0.552 | 0.5 | 0.552 |
| rs2272593 | 31660025 | 103.2 | 0.55 | 0.514 | 0.55 |
| rs3129792 | 29024283 | 103.2 | 0.549 | 0.515 | 0.549 |
| rs1264305 | 30943085 | 111.2 | 0.548 | 0.485 | 0.548 |
| rs4959047 | 30187311 | 111.2 | 0.547 | 0.488 | 0.547 |
| rs4713436 | 31145442 | 111.2 | 0.541 | 0.515 | 0.541 |
| rs7829 | 29684188 | 106.2 | 0.541 | 0.171 | 0.541 |
| rs3094696 | 30132201 | 103.2 | 0.535 | 0.395 | 0.535 |
| rs3828881 | 31150307 | 107.2 | 0.533 | 0.531 | 0.533 |
| rs3094000 | 31542964 | 113.2 | 0.533 | 0.496 | 0.533 |
| rs2855440 | 33131221 | 100.1 | 0.53 | 0.529 | 0.53 |
| rs6457697 | 32982660 | 116.1 | 0.529 | 0.152 | 0.529 |
| rs6919044 | 29072116 | 116.1 | 0.528 | 0.473 | 0.528 |
| rs1013518 | 29970270 | 108.2 | 0.526 | 0.387 | 0.526 |
| rs2144426 | 29407615 | 96.2 | 0.523 | 0.36 | 0.523 |
| rs4267952 | 29115784 | 111.2 | 0.523 | 0.36 | 0.523 |
| rs4947339 | 28978804 | 111.2 | 0.522 | 0.424 | 0.522 |
| rs4618569 | 30626009 | 111.2 | 0.522 | 0.295 | 0.522 |
| rs7767288 | 32831534 | 116.1 | 0.522 | 0.266 | 0.522 |
| rs3752528 | 32935687 | 107.2 | 0.521 | 0.517 | 0.521 |
| rs3956986 | 29491164 | 113.2 | 0.52 | 0.435 | 0.52 |
| rs2285320 | 30701824 | 100.2 | 0.52 | 0.327 | 0.52 |
| rs6938690 | 32832980 | 116.1 | 0.518 | 0.395 | 0.518 |
| rs2532932 | 30956243 | 113.2 | 0.518 | 0.347 | 0.518 |
| rs1634725 | 30752766 | 89.2 | 0.515 | 0.449 | 0.515 |
| rs3117097 | 32384447 | 103.2 | 0.515 | 0.337 | 0.515 |
| rs2621421 | 32735462 | 100.2 | 0.515 | 0.244 | 0.515 |
| rs2076541 | 32087972 | 111.1 | 0.514 | 0.425 | 0.514 |
| rs3094613 | 30931678 | 103.2 | 0.511 | 0.416 | 0.511 |
| rs4711186 | 29138287 | 111.2 | 0.51 | 0.479 | 0.51 |
| rs3130165 | 33131673 | 103.2 | 0.507 | 0.443 | 0.507 |
| rs2395312 | 33059883 | 103.2 | 0.506 | 0.292 | 0.506 |
| rs1627764 | 29957137 | 98.2 | 0.505 | 0.289 | 0.505 |
| rs204896 | 31834855 | 98.2 | 0.505 | 0.195 | 0.505 |
| rs7750714 | 30034531 | 116.1 | 0.502 | 0.264 | 0.502 |
| rs1054684 | 32778457 | 111.2 | 0.501 | 0.191 | 0.501 |
| rs7768931 | 30034555 | 116.1 | 0.5 | 0.316 | 0.5 |
| rs2213573 | 32486779 | 113.2 | 0.499 | 0.434 | 0.499 |
| rs241410 | 32632408 | 110.2 | 0.496 | 0.205 | 0.496 |
| rs2523734 | 30190876 | 100.2 | 0.495 | 0.484 | 0.495 |
| rs2163472 | 32976758 | 113.2 | 0.495 | 0.199 | 0.495 |
| rs6930003 | 29823196 | 116.1 | 0.484 | 0.123 | 0.484 |
| rs4713193 | 29037374 | 111.2 | 0.483 | 0.289 | 0.483 |
| rs1264321 | 30630102 | 110.2 | 0.482 | 0.471 | 0.482 |
| rs204878 | 32071248 | 113.2 | 0.481 | 0.298 | 0.481 |
| rs2301752 | 30100207 | 100.2 | 0.48 | 0.396 | 0.48 |
| rs6901830 | 33099791 | 116.1 | 0.472 | 0.349 | 0.472 |
| rs3998159 | 32452776 | 114.2 | 0.471 | 0.178 | 0.471 |
| rs2246986 | 31540939 | 113.2 | 0.466 | 0.41 | 0.466 |
| rs3117113 | 32096049 | 103.2 | 0.466 | 0.388 | 0.466 |
| rs2857128 | 32778368 | 100.2 | 0.464 | 0.326 | 0.464 |
| rs2516643 | 30566213 | 100.2 | 0.458 | 0.37 | 0.458 |
| rs3117103 | 32369057 | 103.2 | 0.458 | 0.366 | 0.458 |
| rs2233965 | 31141705 | 113.2 | 0.457 | 0.372 | 0.457 |
| rs6916394 | 31522961 | 116.1 | 0.456 | 0.398 | 0.456 |
| rs2074485 | 29665027 | 98.2 | 0.455 | 0.406 | 0.455 |
| rs1619893 | 29837080 | 89.2 | 0.455 | 0.231 | 0.455 |
| rs4449615 | 32719077 | 113.2 | 0.452 | 0.389 | 0.452 |
| rs3115611 | 29622464 | 103.2 | 0.452 | 0.307 | 0.452 |
| rs2301748 | 31142155 | 100.2 | 0.452 | 0.222 | 0.452 |
| rs3129845 | 32167034 | 103.2 | 0.452 | 0.218 | 0.452 |
| rs3130829 | 29298453 | 103.2 | 0.451 | 0.352 | 0.451 |
| rs6457125 | 30040971 | 116.1 | 0.451 | 0.309 | 0.451 |
| rs2571402 | 29703138 | 110.2 | 0.451 | 0.282 | 0.451 |
| rs2157605 | 30514809 | 110.1 | 0.448 | 0.349 | 0.448 |
| rs2275853 | 29747826 | 100.2 | 0.448 | 0.333 | 0.448 |
| rs3132710 | 29907423 | 103.2 | 0.446 | 0.376 | 0.446 |
| rs2854005 | 31375653 | 100.2 | 0.446 | 0.317 | 0.446 |
| rs1011985 | 29453617 | 113.2 | 0.446 | 0.269 | 0.446 |
| rs2075789 | 31767002 | 96.2 | 0.446 | 0.251 | 0.446 |
| rs2076182 | 29462471 | 98.1 | 0.445 | 0.262 | 0.445 |
| rs3132684 | 29761466 | 103.2 | 0.444 | 0.359 | 0.444 |
| rs238877 | 29120249 | 96.2 | 0.444 | 0.327 | 0.444 |
| rs2072681 | 31638157 | 98.1 | 0.444 | 0.315 | 0.444 |
| rs6919908 | 31304263 | 116.1 | 0.443 | 0.344 | 0.443 |
| rs2647045 | 32438857 | 100.2 | 0.443 | 0.324 | 0.443 |
| rs1632986 | 29835241 | 89.2 | 0.441 | 0.398 | 0.441 |
| rs2395470 | 31294570 | 100.2 | 0.441 | 0.357 | 0.441 |
| rs915668 | 29863415 | 110.2 | 0.44 | 0.381 | 0.44 |
| rs2295119 | 33062317 | 116.2 | 0.439 | 0.325 | 0.439 |
| rs2071536 | 32814860 | 98.1 | 0.438 | 0.228 | 0.438 |
| rs3130827 | 29293236 | 103.2 | 0.434 | 0.398 | 0.434 |
| rs3093556 | 31607132 | 111.2 | 0.434 | 0.363 | 0.434 |
| rs3734818 | 29824399 | 107.2 | 0.433 | 0.378 | 0.433 |
| rs4713270 | 29995478 | 111.2 | 0.433 | 0.362 | 0.433 |
| rs1988565 | 32524249 | 113.2 | 0.433 | 0.348 | 0.433 |
| rs1233333 | 29566478 | 89.2 | 0.433 | 0.272 | 0.433 |
| rs2074489 | 31299436 | 98.2 | 0.432 | 0.297 | 0.432 |
| rs1736981 | 29832893 | 89.2 | 0.431 | 0.359 | 0.431 |
| rs3129005 | 29921455 | 103.1 | 0.431 | 0.356 | 0.431 |
| rs3998357 | 31385813 | 108.2 | 0.43 | 0.36 | 0.43 |
| rs3132645 | 30180007 | 103.2 | 0.429 | 0.218 | 0.429 |
| rs1264548 | 30438888 | 92.2 | 0.429 | 0.201 | 0.429 |
| rs2428495 | 31380927 | 110.2 | 0.428 | 0.33 | 0.428 |
| rs3817824 | 29835221 | 107.2 | 0.427 | 0.343 | 0.427 |
| rs1632948 | 29564807 | 89.2 | 0.427 | 0.321 | 0.427 |
| rs1736998 | 29830662 | 89.2 | 0.426 | 0.348 | 0.426 |
| rs2535252 | 29404413 | 100.2 | 0.426 | 0.208 | 0.426 |
| rs1625485 | 29665147 | 98.1 | 0.425 | 0.277 | 0.425 |
| rs1811197 | 31386409 | 92.2 | 0.424 | 0.415 | 0.424 |
| rs2524173 | 30203602 | 100.1 | 0.423 | 0.376 | 0.423 |
| rs6925580 | 32471946 | 116.1 | 0.423 | 0.285 | 0.423 |
| rs3828903 | 31235497 | 107.2 | 0.422 | 0.41 | 0.422 |
| rs2596450 | 31496962 | 113.2 | 0.422 | 0.367 | 0.422 |
| rs6457042 | 29828592 | 116.1 | 0.421 | 0.405 | 0.421 |
| rs1799908 | 33145690 | 102.2 | 0.421 | 0.371 | 0.421 |
| rs8365 | 32168149 | 106.1 | 0.419 | 0.344 | 0.419 |
| rs3823340 | 29683759 | 107.2 | 0.419 | 0.334 | 0.419 |
| rs1611589 | 29912926 | 113.2 | 0.417 | 0.253 | 0.417 |
| rs2071291 | 32137948 | 98.1 | 0.414 | 0.399 | 0.414 |
| rs1233387 | 29618316 | 100.2 | 0.413 | 0.367 | 0.413 |
| rs6920823 | 32553557 | 116.1 | 0.413 | 0.359 | 0.413 |
| rs2251731 | 31138123 | 113.2 | 0.412 | 0.399 | 0.412 |
| rs6920830 | 32553601 | 116.1 | 0.411 | 0.286 | 0.411 |
| rs3873375 | 31310652 | 113.2 | 0.41 | 0.379 | 0.41 |
| rs1737004 | 29823730 | 89.2 | 0.41 | 0.37 | 0.41 |
| rs6457611 | 32626143 | 116.1 | 0.409 | 0.22 | 0.409 |
| rs2524088 | 31303580 | 100.2 | 0.408 | 0.392 | 0.408 |
| rs3749962 | 31807114 | 107.1 | 0.408 | 0.365 | 0.408 |
| rs1737085 | 29487148 | 110.2 | 0.408 | 0.295 | 0.408 |
| rs7382817 | 31529766 | 116.1 | 0.408 | 0.29 | 0.408 |
| rs2394413 | 30843040 | 113.2 | 0.408 | 0.151 | 0.408 |
| rs2073151 | 29427388 | 98.2 | 0.407 | 0.318 | 0.407 |
| rs1058026 | 31380437 | 86.2 | 0.407 | 0.299 | 0.407 |
| rs2272873 | 29834825 | 100.2 | 0.405 | 0.385 | 0.405 |
| rs1632933 | 29862889 | 98.2 | 0.404 | 0.399 | 0.404 |
| rs1632939 | 29567693 | 110.2 | 0.404 | 0.38 | 0.404 |
| rs3132704 | 29910464 | 103.2 | 0.404 | 0.314 | 0.404 |
| rs2523611 | 31380216 | 100.2 | 0.402 | 0.347 | 0.402 |
| rs3823313 | 29489113 | 110.2 | 0.402 | 0.227 | 0.402 |
| rs2524083 | 31301045 | 100.2 | 0.4 | 0.376 | 0.4 |
| rs983561 | 32429437 | 103.2 | 0.989 | 0.649 | 0.989 |
| rs915660 | 30914593 | 113.2 | 0.985 | 0.79 | 0.985 |
| rs1269839 | 32156676 | 103.1 | 0.983 | 0.81 | 0.983 |
| rs2050190 | 32364865 | 113.2 | 0.978 | 0.732 | 0.978 |
| rs8408 | 30638424 | 106.2 | 0.976 | 0.502 | 0.976 |
| rs3115573 | 31989600 | 103.2 | 0.964 | 0.794 | 0.964 |
| rs2230365 | 31296206 | 102.1 | 0.957 | 0.707 | 0.957 |
| rs2284178 | 31202883 | 100.2 | 0.956 | 0.639 | 0.956 |
| rs720467 | 31189621 | 98.2 | 0.955 | 0.715 | 0.955 |
| rs1842399 | 32524070 | 103.2 | 0.954 | 0.572 | 0.954 |
| rs2857106 | 32780985 | 111.2 | 0.951 | 0.928 | 0.951 |
| rs1894552 | 32166222 | 113.2 | 0.947 | 0.698 | 0.947 |
| rs1048664 | 30033270 | 102.2 | 0.946 | 0.801 | 0.946 |
| rs2076529 | 32383441 | 98.2 | 0.944 | 0.653 | 0.944 |
| rs2269422 | 31922051 | 100.1 | 0.941 | 0.799 | 0.941 |
| rs3135338 | 32171974 | 103.2 | 0.941 | 0.799 | 0.941 |
| rs3129907 | 32094502 | 103.2 | 0.939 | 0.726 | 0.939 |
| rs2621413 | 32737322 | 111.2 | 0.938 | 0.832 | 0.938 |
| rs3129848 | 32167806 | 103.2 | 0.936 | 0.894 | 0.936 |
| rs2395253 | 32716730 | 100.1 | 0.927 | 0.682 | 0.927 |
| rs3135365 | 32160012 | 103.2 | 0.924 | 0.716 | 0.924 |
| rs2395106 | 32219244 | 100.2 | 0.921 | 0.794 | 0.921 |
| rs2285796 | 30181652 | 103.2 | 0.921 | 0.744 | 0.921 |
| rs2284177 | 30860353 | 111.2 | 0.919 | 0.679 | 0.919 |
| rs3115563 | 32004395 | 103.2 | 0.914 | 0.877 | 0.914 |
| rs2074477 | 29902793 | 98.2 | 0.909 | 0.826 | 0.909 |
| rs2301755 | 30637376 | 100.2 | 0.908 | 0.756 | 0.908 |
| rs2057727 | 30358231 | 113.2 | 0.906 | 0.552 | 0.906 |
| rs2269705 | 30717985 | 100.2 | 0.905 | 0.725 | 0.905 |
| rs2076177 | 29758367 | 113.2 | 0.904 | 0.832 | 0.904 |
| rs2071285 | 31951188 | 100.2 | 0.9 | 0.8 | 0.9 |
| rs1109146 | 30690786 | 113.2 | 0.896 | 0.886 | 0.896 |
| rs2395178 | 32431144 | 113.2 | 0.895 | 0.811 | 0.895 |
| rs1978029 | 32733277 | 114.2 | 0.886 | 0.695 | 0.886 |
| rs408359 | 32167885 | 100.2 | 0.884 | 0.764 | 0.884 |
| rs2239529 | 30139086 | 100.2 | 0.878 | 0.761 | 0.878 |
| rs2269425 | 32143376 | 100.2 | 0.873 | 0.824 | 0.873 |
| rs2073048 | 32106190 | 98.2 | 0.867 | 0.821 | 0.867 |
| rs3806155 | 32144135 | 107.2 | 0.866 | 0.805 | 0.866 |
| rs436845 | 31968493 | 94.2 | 0.862 | 0.776 | 0.862 |
| rs434841 | 31961798 | 110.1 | 0.857 | 0.717 | 0.857 |
| rs1555115 | 32125277 | 113.2 | 0.847 | 0.827 | 0.847 |
| rs2284174 | 30774302 | 103.2 | 0.844 | 0.808 | 0.844 |
| rs2239806 | 32182064 | 103.2 | 0.844 | 0.722 | 0.844 |
| rs1610714 | 29834795 | 110.2 | 0.84 | 0.76 | 0.84 |
| rs2071289 | 31905893 | 98.2 | 0.834 | 0.695 | 0.834 |
| rs1264302 | 30653392 | 100.2 | 0.828 | 0.748 | 0.828 |
| rs1265099 | 31166240 | 100.1 | 0.824 | 0.263 | 0.824 |
| rs926070 | 32028323 | 86.2 | 0.817 | 0.636 | 0.817 |
| rs1150738 | 30095878 | 110.2 | 0.808 | 0.784 | 0.808 |
| rs2075015 | 30741323 | 98.2 | 0.798 | 0.415 | 0.798 |
| rs1576 | 31171211 | 106.2 | 0.786 | 0.783 | 0.786 |
| rs2074482 | 30097260 | 98.2 | 0.779 | 0.778 | 0.779 |
| rs2076523 | 32141592 | 98.1 | 0.772 | 0.664 | 0.772 |
| rs2395182 | 32154074 | 113.2 | 0.763 | 0.632 | 0.763 |
| rs241429 | 32797253 | 110.2 | 0.724 | 0.705 | 0.724 |
| rs565448 | 32031027 | 83.2 | 0.723 | 0.628 | 0.723 |
| rs1383265 | 32734679 | 113.2 | 0.962 | 0.751 | 0.962 |
| rs1150771 | 30445586 | 100.2 | 0.917 | 0.753 | 0.917 |
| rs2284189 | 32606911 | 116.2 | 0.914 | 0.76 | 0.914 |
| rs3213484 | 32719850 | 106.2 | 0.89 | 0.788 | 0.89 |
| rs2856716 | 32614668 | 100.2 | 0.888 | 0.816 | 0.888 |
| rs2076526 | 32383772 | 98.2 | 0.881 | 0.807 | 0.881 |
| rs3830122 | 32484982 | 107.2 | 0.876 | 0.851 | 0.876 |
| rs1882 | 31153669 | 106.2 | 0.84 | 0.698 | 0.84 |
| rs512559 | 31686820 | 110.1 | 0.825 | 0.621 | 0.825 |
| rs2857102 | 32787184 | 100.2 | 0.794 | 0.757 | 0.794 |
| rs2856698 | 32630912 | 100.1 | 0.766 | 0.672 | 0.766 |
| rs204989 | 31932609 | 87.1 | 0.692 | 0.564 | 0.692 |
| rs419788 | 31699557 | 96.2 | 0.998 | 0.687 | 0.998 |
| rs986475 | 31615386 | 113.2 | 0.993 | 0.775 | 0.993 |
| rs2763979 | 31853239 | 100.2 | 0.993 | 0.727 | 0.993 |
| rs1062470 | 31145238 | 103.2 | 0.992 | 0.593 | 0.992 |
| rs2523668 | 31496815 | 113.2 | 0.989 | 0.696 | 0.989 |
| rs2524069 | 31307104 | 100.2 | 0.988 | 0.751 | 0.988 |
| rs3135339 | 32170018 | 113.2 | 0.988 | 0.722 | 0.988 |
| rs206776 | 32724468 | 87.2 | 0.988 | 0.604 | 0.988 |
| rs1237485 | 28956896 | 108.2 | 0.987 | 0.611 | 0.987 |
| rs805290 | 31707241 | 100.2 | 0.986 | 0.716 | 0.986 |
| rs805268 | 31697015 | 100.1 | 0.986 | 0.658 | 0.986 |
| rs1345228 | 29203145 | 113.2 | 0.984 | 0.678 | 0.984 |
| rs7742523 | 29760409 | 116.1 | 0.983 | 0.724 | 0.983 |
| rs1053924 | 31891472 | 108.1 | 0.981 | 0.726 | 0.981 |
| rs1270942 | 31977681 | 110.1 | 0.981 | 0.68 | 0.981 |
| rs3129872 | 32432939 | 103.2 | 0.98 | 0.723 | 0.98 |
| rs805256 | 31694554 | 100.2 | 0.979 | 0.798 | 0.979 |
| rs3116998 | 33098678 | 103.2 | 0.979 | 0.563 | 0.979 |
| rs2517484 | 31134935 | 100.2 | 0.978 | 0.802 | 0.978 |
| rs886398 | 29905295 | 110.2 | 0.976 | 0.722 | 0.976 |
| rs984778 | 32170845 | 113.2 | 0.976 | 0.701 | 0.976 |
| rs1819784 | 29139038 | 92.2 | 0.971 | 0.69 | 0.971 |
| rs537757 | 32039258 | 108.2 | 0.97 | 0.837 | 0.97 |
| rs213199 | 33237203 | 111.1 | 0.969 | 0.742 | 0.969 |
| rs7762289 | 28972787 | 116.1 | 0.969 | 0.669 | 0.969 |
| rs1264709 | 29827234 | 100.2 | 0.969 | 0.611 | 0.969 |
| rs2071352 | 33037253 | 98.2 | 0.968 | 0.77 | 0.968 |
| rs2213584 | 32184016 | 113.2 | 0.968 | 0.737 | 0.968 |
| rs7382138 | 29496842 | 116.1 | 0.968 | 0.614 | 0.968 |
| rs660550 | 31608035 | 110.1 | 0.967 | 0.713 | 0.967 |
| rs3094626 | 30094381 | 103.2 | 0.966 | 0.777 | 0.966 |
| rs130078 | 31179378 | 103.2 | 0.965 | 0.65 | 0.965 |
| rs1610647 | 29824587 | 89.2 | 0.963 | 0.72 | 0.963 |
| rs2071594 | 31571444 | 103.2 | 0.963 | 0.671 | 0.963 |
| rs393603 | 29948281 | 80.2 | 0.963 | 0.627 | 0.963 |
| rs1034325 | 30139215 | 113.2 | 0.961 | 0.9 | 0.961 |
| rs971570 | 30233675 | 113.2 | 0.961 | 0.836 | 0.961 |
| rs3115635 | 29751251 | 103.2 | 0.961 | 0.825 | 0.961 |
| rs2524172 | 30204369 | 100.2 | 0.961 | 0.822 | 0.961 |
| rs2523746 | 30000843 | 100.2 | 0.961 | 0.675 | 0.961 |
| rs1362069 | 29513210 | 110.2 | 0.961 | 0.664 | 0.961 |
| rs3129203 | 33129204 | 116.2 | 0.959 | 0.748 | 0.959 |
| rs3871248 | 30916749 | 113.2 | 0.958 | 0.922 | 0.958 |
| rs4248157 | 31312901 | 111.2 | 0.958 | 0.876 | 0.958 |
| rs3130140 | 30128538 | 103.2 | 0.958 | 0.857 | 0.958 |
| rs2524100 | 31295077 | 100.2 | 0.958 | 0.827 | 0.958 |
| rs1800625 | 31923199 | 103.1 | 0.958 | 0.675 | 0.958 |
| rs2239709 | 31566344 | 98.2 | 0.957 | 0.808 | 0.957 |
| rs241439 | 32568294 | 110.2 | 0.956 | 0.862 | 0.956 |
| rs6907291 | 32952405 | 116.1 | 0.956 | 0.853 | 0.956 |
| rs6917477 | 30086778 | 116.1 | 0.956 | 0.845 | 0.956 |
| rs6923504 | 32453926 | 116.1 | 0.955 | 0.865 | 0.955 |
| rs6906021 | 32626921 | 116.1 | 0.955 | 0.67 | 0.955 |
| rs2849015 | 32218673 | 100.2 | 0.954 | 0.871 | 0.954 |
| rs2517462 | 30956989 | 100.1 | 0.953 | 0.935 | 0.953 |
| rs7382297 | 31306358 | 116.1 | 0.953 | 0.776 | 0.953 |
| rs6457713 | 33079223 | 116.1 | 0.953 | 0.743 | 0.953 |
| rs1075496 | 30719377 | 103.2 | 0.953 | 0.731 | 0.953 |
| rs3131083 | 29102525 | 103.2 | 0.952 | 0.823 | 0.952 |
| rs2256965 | 31613811 | 113.2 | 0.952 | 0.778 | 0.952 |
| rs3093662 | 31602870 | 103.2 | 0.951 | 0.819 | 0.951 |
| rs2395305 | 32978759 | 113.2 | 0.951 | 0.713 | 0.951 |
| rs3763304 | 32140112 | 107.2 | 0.95 | 0.896 | 0.95 |
| rs2227126 | 32486530 | 96.2 | 0.95 | 0.882 | 0.95 |
| rs261946 | 30042092 | 89.1 | 0.95 | 0.867 | 0.95 |
| rs4987411 | 29427080 | 113.2 | 0.949 | 0.775 | 0.949 |
| rs1633017 | 29853725 | 111.2 | 0.949 | 0.686 | 0.949 |
| rs7382061 | 30108754 | 116.1 | 0.949 | 0.564 | 0.949 |
| rs3129980 | 30529129 | 103.2 | 0.948 | 0.747 | 0.948 |
| rs3734838 | 30140998 | 107.1 | 0.947 | 0.85 | 0.947 |
| rs3129073 | 29386580 | 103.2 | 0.946 | 0.889 | 0.946 |
| rs1063355 | 32398471 | 106.2 | 0.946 | 0.676 | 0.946 |
| rs994321 | 29143111 | 113.2 | 0.946 | 0.653 | 0.946 |
| rs3129809 | 30106379 | 103.2 | 0.945 | 0.764 | 0.945 |
| rs7766854 | 32456487 | 116.1 | 0.945 | 0.691 | 0.945 |
| rs3763288 | 31141125 | 107.2 | 0.944 | 0.899 | 0.944 |
| rs630379 | 31693012 | 110.1 | 0.944 | 0.82 | 0.944 |
| rs2844845 | 29777998 | 100.2 | 0.944 | 0.814 | 0.944 |
| rs3130666 | 30510918 | 103.2 | 0.944 | 0.804 | 0.944 |
| rs2021723 | 30165122 | 113.2 | 0.944 | 0.771 | 0.944 |
| rs1634718 | 30743623 | 94.2 | 0.943 | 0.84 | 0.943 |
| rs1264539 | 30449418 | 100.1 | 0.942 | 0.753 | 0.942 |
| rs2844748 | 30108896 | 100.2 | 0.942 | 0.687 | 0.942 |
| rs805292 | 31748849 | 113.2 | 0.941 | 0.777 | 0.941 |
| rs7741884 | 29831007 | 116.1 | 0.941 | 0.565 | 0.941 |
| rs2245420 | 29853446 | 100.2 | 0.94 | 0.887 | 0.94 |
| rs259940 | 29782692 | 110.2 | 0.94 | 0.787 | 0.94 |
| rs2853935 | 31313170 | 100.2 | 0.94 | 0.784 | 0.94 |
| rs6911467 | 30032851 | 116.1 | 0.94 | 0.565 | 0.94 |
| rs3132712 | 29909461 | 103.2 | 0.939 | 0.843 | 0.939 |
| rs3763317 | 32147545 | 107.2 | 0.939 | 0.774 | 0.939 |
| rs2853939 | 31309934 | 100.2 | 0.938 | 0.696 | 0.938 |
| rs605203 | 31906001 | 100.1 | 0.937 | 0.866 | 0.937 |
| rs6457594 | 32469713 | 116.1 | 0.937 | 0.802 | 0.937 |
| rs2144014 | 33058844 | 96.2 | 0.937 | 0.574 | 0.937 |
| rs2534790 | 29294926 | 100.2 | 0.936 | 0.907 | 0.936 |
| rs986522 | 33128684 | 86.2 | 0.936 | 0.737 | 0.936 |
| rs805264 | 31682556 | 100.2 | 0.936 | 0.71 | 0.936 |
| rs7757269 | 29458642 | 116.1 | 0.935 | 0.83 | 0.935 |
| rs916570 | 29836789 | 110.2 | 0.934 | 0.831 | 0.934 |
| rs3093668 | 31605176 | 111.1 | 0.933 | 0.836 | 0.933 |
| rs928824 | 29995647 | 110.2 | 0.93 | 0.793 | 0.93 |
| rs3132682 | 30105177 | 103.2 | 0.93 | 0.783 | 0.93 |
| rs4248148 | 30512892 | 111.2 | 0.93 | 0.781 | 0.93 |
| rs707928 | 31801226 | 113.2 | 0.93 | 0.717 | 0.93 |
| rs3130375 | 30092490 | 103.2 | 0.93 | 0.711 | 0.93 |
| rs3097644 | 32716287 | 110.2 | 0.929 | 0.912 | 0.929 |
| rs1233397 | 29613589 | 100.2 | 0.928 | 0.824 | 0.928 |
| rs2023371 | 30142873 | 94.2 | 0.928 | 0.795 | 0.928 |
| rs2071540 | 32806329 | 111.2 | 0.928 | 0.546 | 0.928 |
| rs4713603 | 32972991 | 116.2 | 0.927 | 0.823 | 0.927 |
| rs3094014 | 31492277 | 103.2 | 0.927 | 0.784 | 0.927 |
| rs2856726 | 32437478 | 100.2 | 0.927 | 0.726 | 0.927 |
| rs2735005 | 29579222 | 100.2 | 0.927 | 0.529 | 0.927 |
| rs241414 | 32628558 | 87.2 | 0.926 | 0.811 | 0.926 |
| rs2516400 | 31540008 | 111.2 | 0.926 | 0.556 | 0.926 |
| rs2517416 | 30726687 | 100.2 | 0.923 | 0.806 | 0.923 |
| rs3749971 | 29113530 | 107.2 | 0.922 | 0.84 | 0.922 |
| rs6928431 | 30085178 | 116.1 | 0.922 | 0.768 | 0.922 |
| rs6911693 | 31548555 | 116.1 | 0.922 | 0.71 | 0.922 |
| rs1269852 | 31850948 | 100.2 | 0.922 | 0.638 | 0.922 |
| rs241457 | 32902901 | 100.2 | 0.921 | 0.883 | 0.921 |
| rs2077102 | 31382598 | 113.2 | 0.921 | 0.632 | 0.921 |
| rs1736995 | 29829076 | 101.2 | 0.921 | 0.436 | 0.921 |
| rs241413 | 32629757 | 87.2 | 0.92 | 0.863 | 0.92 |
| rs1362074 | 29451104 | 88.2 | 0.92 | 0.619 | 0.92 |
| rs2524222 | 30571886 | 100.2 | 0.919 | 0.893 | 0.919 |
| rs2857768 | 29405269 | 100.2 | 0.919 | 0.825 | 0.919 |
| rs2394390 | 30372579 | 103.2 | 0.919 | 0.592 | 0.919 |
| rs3218819 | 30650855 | 106.1 | 0.918 | 0.665 | 0.918 |
| rs2736176 | 31646242 | 110.2 | 0.917 | 0.785 | 0.917 |
| rs885933 | 29905601 | 108.2 | 0.917 | 0.778 | 0.917 |
| rs2517889 | 29865959 | 100.2 | 0.916 | 0.781 | 0.916 |
| rs2207337 | 28971000 | 113.2 | 0.916 | 0.553 | 0.916 |
| rs4713293 | 30163613 | 111.2 | 0.914 | 0.909 | 0.914 |
| rs2040410 | 32608410 | 94.2 | 0.914 | 0.525 | 0.914 |
| rs1557611 | 30179562 | 113.2 | 0.914 | 0.517 | 0.914 |
| rs3129942 | 32109040 | 103.2 | 0.913 | 0.884 | 0.913 |
| rs3094138 | 29969774 | 111.2 | 0.913 | 0.801 | 0.913 |
| rs3129304 | 32975190 | 116.2 | 0.913 | 0.801 | 0.913 |
| rs2071288 | 32169006 | 98.1 | 0.913 | 0.731 | 0.913 |
| rs6921644 | 33090551 | 116.1 | 0.913 | 0.645 | 0.913 |
| rs206777 | 32723303 | 87.2 | 0.911 | 0.864 | 0.911 |
| rs17719 | 30771753 | 106.2 | 0.91 | 0.587 | 0.91 |
| rs1024470 | 29428814 | 113.2 | 0.909 | 0.837 | 0.909 |
| rs620202 | 32935333 | 113.1 | 0.909 | 0.761 | 0.909 |
| rs409558 | 31766821 | 98.2 | 0.909 | 0.484 | 0.909 |
| rs3117330 | 29288348 | 103.1 | 0.907 | 0.781 | 0.907 |
| rs2071481 | 32813278 | 98.2 | 0.907 | 0.763 | 0.907 |
| rs2071544 | 32807641 | 111.2 | 0.907 | 0.729 | 0.907 |
| rs362512 | 29593084 | 111.2 | 0.906 | 0.899 | 0.906 |
| rs1056032 | 28933525 | 111.2 | 0.906 | 0.794 | 0.906 |
| rs4711185 | 29424044 | 111.2 | 0.906 | 0.777 | 0.906 |
| rs6457204 | 30378674 | 116.1 | 0.905 | 0.899 | 0.905 |
| rs7451190 | 31235347 | 116.1 | 0.905 | 0.747 | 0.905 |
| rs474534 | 31708865 | 94.1 | 0.905 | 0.589 | 0.905 |
| rs1480380 | 32906657 | 100.2 | 0.905 | 0.571 | 0.905 |
| rs6914285 | 31544258 | 116.1 | 0.904 | 0.885 | 0.904 |
| rs3129953 | 32387571 | 103.2 | 0.904 | 0.839 | 0.904 |
| rs6927487 | 29996800 | 116.1 | 0.903 | 0.805 | 0.903 |
| rs3095151 | 30960949 | 111.2 | 0.903 | 0.74 | 0.903 |
| rs1573298 | 30176932 | 113.2 | 0.902 | 0.888 | 0.902 |
| rs3104395 | 32456183 | 103.2 | 0.902 | 0.883 | 0.902 |
| rs2395316 | 33058707 | 100.2 | 0.902 | 0.756 | 0.902 |
| rs1264707 | 29833845 | 100.2 | 0.901 | 0.888 | 0.901 |
| rs3095313 | 30861357 | 103.2 | 0.901 | 0.788 | 0.901 |
| rs2735054 | 29471373 | 110.2 | 0.901 | 0.754 | 0.901 |
| rs886390 | 30105752 | 111.2 | 0.901 | 0.731 | 0.901 |
| rs2523721 | 30227002 | 100.2 | 0.9 | 0.887 | 0.9 |
| rs2239800 | 32708054 | 98.2 | 0.9 | 0.865 | 0.9 |
| rs2747466 | 29429327 | 100.2 | 0.9 | 0.651 | 0.9 |
| rs1029239 | 29908920 | 98.2 | 0.9 | 0.573 | 0.9 |
| rs2523676 | 31494915 | 113.2 | 0.899 | 0.888 | 0.899 |
| rs3757335 | 30516263 | 107.2 | 0.899 | 0.836 | 0.899 |
| rs760070 | 31690714 | 113.1 | 0.899 | 0.745 | 0.899 |
| rs2442719 | 31379290 | 100.2 | 0.899 | 0.357 | 0.899 |
| rs1573297 | 30177113 | 113.2 | 0.898 | 0.843 | 0.898 |
| rs3132569 | 31162252 | 113.2 | 0.898 | 0.803 | 0.898 |
| rs2844523 | 31139346 | 110.2 | 0.898 | 0.598 | 0.898 |
| rs2077573 | 30199109 | 96.2 | 0.897 | 0.845 | 0.897 |
| rs2249168 | 30729012 | 111.2 | 0.895 | 0.842 | 0.895 |
| rs2844780 | 29975284 | 113.2 | 0.895 | 0.791 | 0.895 |
| rs6457655 | 32737067 | 116.1 | 0.895 | 0.586 | 0.895 |
| rs7770024 | 32724907 | 116.1 | 0.895 | 0.495 | 0.895 |
| rs458434 | 33244273 | 87.2 | 0.894 | 0.872 | 0.894 |
| rs204894 | 32113647 | 96.1 | 0.894 | 0.855 | 0.894 |
| rs6905949 | 30201287 | 116.1 | 0.894 | 0.788 | 0.894 |
| rs3135376 | 32404946 | 103.2 | 0.894 | 0.784 | 0.894 |
| rs1018434 | 32052117 | 111.2 | 0.893 | 0.873 | 0.893 |
| rs204879 | 32062890 | 96.2 | 0.893 | 0.864 | 0.893 |
| rs3129943 | 32109452 | 103.2 | 0.893 | 0.83 | 0.893 |
| rs7772569 | 32707547 | 116.1 | 0.893 | 0.728 | 0.893 |
| rs2248902 | 31293436 | 108.2 | 0.892 | 0.878 | 0.892 |
| rs2517449 | 30980505 | 100.1 | 0.892 | 0.773 | 0.892 |
| rs2859448 | 31495197 | 113.1 | 0.892 | 0.674 | 0.892 |
| rs2857694 | 31646551 | 110.2 | 0.892 | 0.658 | 0.892 |
| rs3132671 | 29949045 | 103.2 | 0.891 | 0.5 | 0.891 |
| rs2395301 | 32970142 | 113.1 | 0.89 | 0.8 | 0.89 |
| rs2516455 | 31191330 | 100.2 | 0.89 | 0.681 | 0.89 |
| rs2294472 | 33091881 | 100.2 | 0.889 | 0.883 | 0.889 |
| rs2077580 | 31791601 | 96.2 | 0.887 | 0.661 | 0.887 |
| rs1061783 | 33284075 | 106.2 | 0.886 | 0.848 | 0.886 |
| rs3135024 | 33040530 | 103.2 | 0.885 | 0.725 | 0.885 |
| rs204994 | 31925755 | 87.1 | 0.885 | 0.651 | 0.885 |
| rs1059174 | 29756938 | 106.2 | 0.885 | 0.618 | 0.885 |
| rs1233422 | 29568269 | 100.2 | 0.884 | 0.718 | 0.884 |
| rs7772297 | 31387575 | 116.1 | 0.883 | 0.651 | 0.883 |
| rs3132613 | 30598323 | 103.2 | 0.883 | 0.351 | 0.883 |
| rs2272592 | 31757189 | 103.2 | 0.881 | 0.801 | 0.881 |
| rs2523790 | 29866221 | 100.2 | 0.881 | 0.75 | 0.881 |
| rs2071476 | 32818792 | 96.2 | 0.881 | 0.703 | 0.881 |
| rs2105903 | 32166413 | 113.2 | 0.878 | 0.873 | 0.878 |
| rs7382555 | 29827043 | 116.1 | 0.878 | 0.848 | 0.878 |
| rs3115663 | 31660524 | 103.2 | 0.878 | 0.839 | 0.878 |
| rs492899 | 31992272 | 113.1 | 0.878 | 0.753 | 0.878 |
| rs707939 | 31497446 | 110.2 | 0.877 | 0.811 | 0.877 |
| rs3094614 | 29952033 | 103.2 | 0.877 | 0.506 | 0.877 |
| rs2734990 | 29583263 | 113.2 | 0.876 | 0.855 | 0.876 |
| rs2273612 | 31684017 | 100.2 | 0.875 | 0.748 | 0.875 |
| rs438999 | 31699064 | 113.1 | 0.875 | 0.727 | 0.875 |
| rs206786 | 32705936 | 108.2 | 0.875 | 0.539 | 0.875 |
| rs362536 | 29297698 | 102.2 | 0.873 | 0.853 | 0.873 |
| rs2523395 | 29473268 | 113.2 | 0.873 | 0.82 | 0.873 |
| rs1064190 | 31135921 | 106.2 | 0.873 | 0.7 | 0.873 |
| rs3130267 | 33308246 | 116.2 | 0.873 | 0.693 | 0.873 |
| rs7762933 | 31223348 | 116.1 | 0.872 | 0.774 | 0.872 |
| rs3094211 | 31150168 | 110.2 | 0.872 | 0.603 | 0.872 |
| rs3129932 | 32106884 | 103.1 | 0.871 | 0.798 | 0.871 |
| rs2275855 | 29747547 | 100.2 | 0.871 | 0.791 | 0.871 |
| rs2516399 | 31540203 | 113.2 | 0.871 | 0.669 | 0.871 |
| rs2857210 | 32736533 | 100.2 | 0.871 | 0.644 | 0.871 |
| rs7357044 | 29387762 | 116.1 | 0.87 | 0.797 | 0.87 |
| rs592229 | 31701199 | 110.2 | 0.87 | 0.701 | 0.87 |
| rs2844773 | 29978253 | 100.2 | 0.87 | 0.527 | 0.87 |
| rs2523459 | 31136066 | 113.2 | 0.869 | 0.635 | 0.869 |
| rs2734962 | 29909184 | 100.2 | 0.868 | 0.843 | 0.868 |
| rs2894046 | 30842842 | 113.2 | 0.868 | 0.808 | 0.868 |
| rs15574 | 31745179 | 106.2 | 0.867 | 0.797 | 0.867 |
| rs6919855 | 32454751 | 116.1 | 0.867 | 0.73 | 0.867 |
| rs1362076 | 29211924 | 113.2 | 0.867 | 0.656 | 0.867 |
| rs740884 | 29637083 | 86.1 | 0.866 | 0.61 | 0.866 |
| rs6922512 | 32973202 | 116.1 | 0.864 | 0.827 | 0.864 |
| rs6926737 | 32401481 | 116.1 | 0.863 | 0.797 | 0.863 |
| rs2523747 | 29999696 | 100.2 | 0.863 | 0.779 | 0.863 |
| rs2023470 | 30177774 | 111.2 | 0.862 | 0.833 | 0.862 |
| rs2239839 | 33280776 | 116.2 | 0.861 | 0.764 | 0.861 |
| rs3131050 | 30530783 | 103.2 | 0.861 | 0.762 | 0.861 |
| rs2523984 | 29852761 | 100.2 | 0.861 | 0.372 | 0.861 |
| rs3132965 | 32166743 | 103.1 | 0.86 | 0.809 | 0.86 |
| rs3115537 | 31556562 | 103.2 | 0.859 | 0.453 | 0.859 |
| rs2647044 | 32438667 | 100.2 | 0.858 | 0.805 | 0.858 |
| rs1419640 | 29121609 | 88.1 | 0.858 | 0.749 | 0.858 |
| rs8283 | 31854057 | 108.2 | 0.857 | 0.807 | 0.857 |
| rs2517810 | 30009796 | 113.2 | 0.857 | 0.758 | 0.857 |
| rs2394694 | 29852570 | 100.2 | 0.856 | 0.814 | 0.856 |
| rs2076483 | 29342303 | 96.2 | 0.854 | 0.707 | 0.854 |
| rs411337 | 31848137 | 96.2 | 0.853 | 0.744 | 0.853 |
| rs6934993 | 29400086 | 116.1 | 0.853 | 0.72 | 0.853 |
| rs1245371 | 30099141 | 100.2 | 0.852 | 0.728 | 0.852 |
| rs6902723 | 32733527 | 116.1 | 0.85 | 0.754 | 0.85 |
| rs3134942 | 32188525 | 103.2 | 0.85 | 0.664 | 0.85 |
| rs454212 | 31993193 | 113.1 | 0.849 | 0.819 | 0.849 |
| rs1362126 | 29461777 | 113.2 | 0.849 | 0.483 | 0.849 |
| rs2075701 | 31707388 | 98.2 | 0.848 | 0.809 | 0.848 |
| rs3130502 | 31200498 | 103.2 | 0.846 | 0.564 | 0.846 |
| rs1057373 | 32806692 | 110.2 | 0.845 | 0.818 | 0.845 |
| rs1736936 | 29565075 | 110.2 | 0.845 | 0.798 | 0.845 |
| rs7763159 | 30128438 | 116.1 | 0.845 | 0.507 | 0.845 |
| rs2517755 | 29667417 | 111.2 | 0.845 | 0.433 | 0.845 |
| rs3131000 | 30862031 | 110.2 | 0.844 | 0.81 | 0.844 |
| rs1233384 | 29621747 | 111.2 | 0.844 | 0.762 | 0.844 |
| rs2239804 | 32182280 | 98.2 | 0.844 | 0.601 | 0.844 |
| rs3132668 | 29957922 | 103.2 | 0.842 | 0.81 | 0.842 |
| rs2074464 | 29470953 | 103.2 | 0.842 | 0.7 | 0.842 |
| rs2857107 | 32778930 | 100.2 | 0.842 | 0.56 | 0.842 |
| rs1362063 | 29430291 | 88.2 | 0.841 | 0.825 | 0.841 |
| rs1536215 | 28930465 | 113.2 | 0.841 | 0.76 | 0.841 |
| rs3135395 | 32175949 | 103.2 | 0.841 | 0.751 | 0.841 |
| rs3757328 | 30089146 | 107.2 | 0.841 | 0.733 | 0.841 |
| rs3130982 | 31144878 | 103.2 | 0.839 | 0.727 | 0.839 |
| rs2395269 | 32819295 | 111.2 | 0.838 | 0.838 | 0.838 |
| rs1264423 | 30632589 | 100.2 | 0.837 | 0.797 | 0.837 |
| rs2532929 | 30958573 | 100.2 | 0.837 | 0.769 | 0.837 |
| rs805303 | 31387124 | 100.2 | 0.837 | 0.726 | 0.837 |
| rs2260000 | 31652156 | 100.2 | 0.837 | 0.577 | 0.837 |
| rs86567 | 32970141 | 113.2 | 0.835 | 0.749 | 0.835 |
| rs3117328 | 29293130 | 103.2 | 0.833 | 0.745 | 0.833 |
| rs4711203 | 29950631 | 111.2 | 0.832 | 0.787 | 0.832 |
| rs2763982 | 31643309 | 111.2 | 0.832 | 0.758 | 0.832 |
| rs3130349 | 32167442 | 106.1 | 0.832 | 0.607 | 0.832 |
| rs2021365 | 29977821 | 94.1 | 0.831 | 0.597 | 0.831 |
| rs3117231 | 33067896 | 103.2 | 0.829 | 0.806 | 0.829 |
| rs2021719 | 30510611 | 113.2 | 0.828 | 0.76 | 0.828 |
| rs5009557 | 32550753 | 113.2 | 0.828 | 0.729 | 0.828 |
| rs1269556 | 30179545 | 100.2 | 0.828 | 0.646 | 0.828 |
| rs3131785 | 30962694 | 103.2 | 0.827 | 0.811 | 0.827 |
| rs362521 | 29619211 | 102.2 | 0.826 | 0.768 | 0.826 |
| rs644774 | 31609248 | 110.2 | 0.826 | 0.725 | 0.826 |
| rs558702 | 31641084 | 113.2 | 0.826 | 0.639 | 0.826 |
| rs3132571 | 30676071 | 113.1 | 0.826 | 0.625 | 0.826 |
| rs6932290 | 32486963 | 116 1 | 0.825 | 0.808 | 0.825 |
| rs429608 | 31701220 | 110.2 | 0.825 | 0.743 | 0.825 |
| rs2245738 | 31292526 | 111.2 | 0.824 | 0.818 | 0.824 |
| rs1633034 | 29514321 | 100.2 | 0.823 | 0.622 | 0.823 |
| rs2524179 | 30196910 | 100.2 | 0.823 | 0.434 | 0.823 |
| rs7776164 | 29021007 | 116.1 | 0.822 | 0.438 | 0.822 |
| rs3095226 | 31550767 | 103.2 | 0.821 | 0.585 | 0.821 |
| rs6457044 | 29835120 | 116.1 | 0.819 | 0.771 | 0.819 |
| rs362515 | 29340290 | 102.2 | 0.819 | 0.761 | 0.819 |
| rs3131934 | 30702602 | 103.2 | 0.818 | 0.751 | 0.818 |
| rs750332 | 31377808 | 102.2 | 0.818 | 0.668 | 0.818 |
| rs5009448 | 30000885 | 113.2 | 0.817 | 0.704 | 0.817 |
| rs2508024 | 30236270 | 100.2 | 0.816 | 0.8 | 0.816 |
| rs3130275 | 33333588 | 116.2 | 0.816 | 0.74 | 0.816 |
| rs3131115 | 30529524 | 103.1 | 0.816 | 0.67 | 0.816 |
| rs1131541 | 32432049 | 108.2 | 0.816 | 0.578 | 0.816 |
| rs7452206 | 31237046 | 116.1 | 0.816 | 0.181 | 0.816 |
| rs2074470 | 29456852 | 103.2 | 0.815 | 0.801 | 0.815 |
| rs7751953 | 33034798 | 116.1 | 0.815 | 0.788 | 0.815 |
| rs2073148 | 29200925 | 98.2 | 0.815 | 0.729 | 0.815 |
| rs885910 | 30619882 | 113.2 | 0.815 | 0.663 | 0.815 |
| rs3094179 | 29586329 | 103.2 | 0.815 | 0.617 | 0.815 |
| rs2301277 | 30773049 | 100.2 | 0.815 | 0.616 | 0.815 |
| rs2239885 | 30442007 | 98.2 | 0.814 | 0.625 | 0.814 |
| rs356963 | 30030521 | 110.2 | 0.813 | 0.743 | 0.813 |
| rs3132721 | 29904076 | 113.2 | 0.813 | 0.705 | 0.813 |
| rs928976 | 33042275 | 104.2 | 0.813 | 0.661 | 0.813 |
| rs3131784 | 30964747 | 103.2 | 0.812 | 0.709 | 0.812 |
| rs3097652 | 33059282 | 103.2 | 0.812 | 0.645 | 0.812 |
| rs805304 | 31756925 | 100.2 | 0.812 | 0.608 | 0.812 |
| rs5017401 | 31375850 | 113.2 | 0.811 | 0.337 | 0.811 |
| rs130067 | 31179324 | 100.2 | 0.81 | 0.746 | 0.81 |
| rs2530716 | 30748954 | 110.2 | 0.81 | 0.545 | 0.81 |
| rs635348 | 31673047 | 113.2 | 0.808 | 0.781 | 0.808 |
| rs464865 | 33258634 | 87.1 | 0.808 | 0.776 | 0.808 |
| rs2844688 | 30713042 | 100.2 | 0.808 | 0.769 | 0.808 |
| rs1632938 | 29567744 | 110.2 | 0.808 | 0.762 | 0.808 |
| rs565876 | 32720807 | 83.2 | 0.808 | 0.746 | 0.808 |
| rs6911246 | 29579052 | 116.1 | 0.808 | 0.583 | 0.808 |
| rs3093557 | 31606877 | 103.2 | 0.808 | 0.558 | 0.808 |
| rs2516672 | 30191144 | 113.2 | 0.807 | 0.679 | 0.807 |
| rs3095307 | 30862807 | 113.2 | 0.807 | 0.594 | 0.807 |
| rs3814959 | 30655945 | 107.2 | 0.806 | 0.66 | 0.806 |
| rs1611493 | 29948942 | 89.2 | 0.806 | 0.613 | 0.806 |
| rs1233492 | 29520905 | 100.2 | 0.806 | 0.561 | 0.806 |
| rs6456890 | 29077674 | 116.1 | 0.804 | 0.734 | 0.804 |
| rs2844680 | 30717254 | 110.1 | 0.804 | 0.436 | 0.804 |
| rs769177 | 31606292 | 111.2 | 0.803 | 0.8 | 0.803 |
| rs2844659 | 30595290 | 110.2 | 0.803 | 0.794 | 0.803 |
| rs2516698 | 30003132 | 100.2 | 0.803 | 0.752 | 0.803 |
| rs204890 | 31856355 | 113.2 | 0.802 | 0.78 | 0.802 |
| rs6457699 | 32983094 | 116.1 | 0.8 | 0.721 | 0.8 |
| rs2071787 | 28974897 | 96.2 | 0.8 | 0.652 | 0.8 |
| rs892666 | 29695486 | 110.2 | 0.798 | 0.781 | 0.798 |
| rs2517894 | 29863400 | 100.2 | 0.798 | 0.775 | 0.798 |
| rs1264348 | 30569231 | 111.2 | 0.798 | 0.716 | 0.798 |
| rs2394734 | 29817004 | 111.2 | 0.797 | 0.792 | 0.797 |
| rs3749950 | 31536261 | 107.2 | 0.797 | 0.635 | 0.797 |
| rs1264350 | 30567303 | 100.2 | 0.797 | 0.409 | 0.797 |
| rs2517407 | 30837226 | 100.2 | 0.796 | 0.769 | 0.796 |
| rs3117426 | 29334582 | 103.2 | 0.796 | 0.751 | 0.796 |
| rs3134767 | 30980618 | 113.2 | 0.796 | 0.287 | 0.796 |
| rs2535238 | 29707557 | 110.2 | 0.795 | 0.598 | 0.795 |
| rs805282 | 31430489 | 100.2 | 0.795 | 0.554 | 0.795 |
| rs401618 | 30010592 | 100.2 | 0.794 | 0.749 | 0.794 |
| rs550605 | 31677905 | 83.2 | 0.794 | 0.583 | 0.794 |
| rs2021722 | 30234862 | 113.2 | 0.792 | 0.776 | 0.792 |
| rs885950 | 31200980 | 113.2 | 0.791 | 0.791 | 0.791 |
| rs2516489 | 31546776 | 100.2 | 0.791 | 0.765 | 0.791 |
| rs2239842 | 33254570 | 103.2 | 0.791 | 0.752 | 0.791 |
| rs3117235 | 33066890 | 103.2 | 0.79 | 0.739 | 0.79 |
| rs446145 | 29291545 | 110.1 | 0.789 | 0.736 | 0.789 |
| rs2894260 | 32466535 | 101.2 | 0.789 | 0.675 | 0.789 |
| rs2143468 | 32079760 | 113.2 | 0.789 | 0.526 | 0.789 |
| rs3873260 | 29584440 | 113.2 | 0.788 | 0.402 | 0.788 |
| rs1050391 | 32911271 | 106.2 | 0.786 | 0.76 | 0.786 |
| rs3094001 | 31542773 | 113.2 | 0.786 | 0.597 | 0.786 |
| rs715044 | 29661663 | 113.2 | 0.786 | 0.296 | 0.786 |
| rs1610640 | 29821044 | 113.2 | 0.785 | 0.752 | 0.785 |
| rs887468 | 30912281 | 100.2 | 0.784 | 0.768 | 0.784 |
| rs755714 | 31380571 | 113.2 | 0.783 | 0.772 | 0.783 |
| rs1265777 | 32043915 | 87.2 | 0.783 | 0.477 | 0.783 |
| rs999265 | 29104351 | 113.2 | 0.783 | 0.41 | 0.783 |
| rs2021729 | 29200147 | 100.2 | 0.782 | 0.734 | 0.782 |
| rs7452076 | 32440830 | 116.1 | 0.781 | 0.757 | 0.781 |
| rs743399 | 31520562 | 86.2 | 0.78 | 0.62 | 0.78 |
| rs2256594 | 32206610 | 100.2 | 0.779 | 0.727 | 0.779 |
| rs1046080 | 31654562 | 106.2 | 0.779 | 0.65 | 0.779 |
| rs2523807 | 29910648 | 116.2 | 0.778 | 0.661 | 0.778 |
| rs3094647 | 29623391 | 103.2 | 0.776 | 0.668 | 0.776 |
| rs421446 | 33176230 | 80.2 | 0.776 | 0.646 | 0.776 |
| rs6415110 | 29708688 | 116.1 | 0.774 | 0.74 | 0.774 |
| rs2285805 | 30104026 | 100.2 | 0.773 | 0.64 | 0.773 |
| rs2242955 | 31533016 | 100.1 | 0.771 | 0.729 | 0.771 |
| rs29233 | 29381987 | 102.2 | 0.771 | 0.616 | 0.771 |
| rs4711312 | 32808072 | 111.2 | 0.77 | 0.428 | 0.77 |
| rs1264327 | 30621340 | 100.2 | 0.768 | 0.629 | 0.768 |
| rs2261033 | 31662272 | 111.1 | 0.767 | 0.712 | 0.767 |
| rs805288 | 31736712 | 100.2 | 0.766 | 0.757 | 0.766 |
| rs3106189 | 33283449 | 103.1 | 0.766 | 0.644 | 0.766 |
| rs2157338 | 32465081 | 113.2 | 0.766 | 0.643 | 0.766 |
| rs805299 | 31678336 | 100.1 | 0.766 | 0.635 | 0.766 |
| rs2074505 | 30581854 | 100.2 | 0.765 | 0.752 | 0.765 |
| rs1042338 | 29923547 | 100.2 | 0.765 | 0.706 | 0.765 |
| rs1059510 | 30518462 | 102.2 | 0.765 | 0.526 | 0.765 |
| rs4678 | 30954740 | 106.2 | 0.764 | 0.743 | 0.764 |
| rs396090 | 32970917 | 111.2 | 0.764 | 0.673 | 0.764 |
| rs1233419 | 29566951 | 100.2 | 0.764 | 0.42 | 0.764 |
| rs1265063 | 31138744 | 100.2 | 0.763 | 0.738 | 0.763 |
| rs3117223 | 33061511 | 103.2 | 0.763 | 0.728 | 0.763 |
| rs2269475 | 31642614 | 100.2 | 0.763 | 0.642 | 0.763 |
| rs2235499 | 33131411 | 98.2 | 0.763 | 0.592 | 0.763 |
| rs6457020 | 29776936 | 116.1 | 0.762 | 0.715 | 0.762 |
| rs185819 | 32076100 | 113.1 | 0.76 | 0.691 | 0.76 |
| rs2273611 | 31739063 | 100.2 | 0.759 | 0.689 | 0.759 |
| rs1632879 | 29978524 | 89.1 | 0.758 | 0.688 | 0.758 |
| rs3093551 | 31609316 | 103.2 | 0.757 | 0.686 | 0.757 |
| rs130072 | 31173304 | 78.2 | 0.757 | 0.522 | 0.757 |
| rs2295667 | 31688856 | 100.2 | 0.756 | 0.55 | 0.756 |
| rs2517466 | 30951047 | 113.2 | 0.753 | 0.747 | 0.753 |
| rs2844724 | 30529923 | 100.1 | 0.753 | 0.285 | 0.753 |
| rs7769073 | 31613471 | 116.1 | 0.751 | 0.434 | 0.751 |
| rs3130780 | 30645066 | 103.2 | 0.75 | 0.7 | 0.75 |
| rs4713354 | 30746135 | 111.2 | 0.747 | 0.734 | 0.747 |
| rs2021720 | 30509976 | 94.2 | 0.747 | 0.481 | 0.747 |
| rs6456879 | 29025153 | 116.1 | 0.747 | 0.466 | 0.747 |
| rs6913528 | 29710985 | 116.1 | 0.746 | 0.578 | 0.746 |
| rs6929434 | 31188789 | 116.1 | 0.746 | 0.502 | 0.746 |
| rs2074499 | 31300023 | 96.2 | 0.745 | 0.72 | 0.745 |
| rs518374 | 32285554 | 83.2 | 0.743 | 0.581 | 0.743 |
| rs446735 | 33247802 | 98.1 | 0.742 | 0.742 | 0.742 |
| rs2075799 | 31549287 | 98.2 | 0.741 | 0.432 | 0.741 |
| rs7757012 | 31169383 | 116.1 | 0.74 | 0.628 | 0.74 |
| rs3093984 | 31554098 | 103.1 | 0.739 | 0.691 | 0.739 |
| rs805258 | 31692396 | 100.2 | 0.737 | 0.719 | 0.737 |
| rs2071351 | 33036995 | 101.2 | 0.737 | 0.703 | 0.737 |
| rs6938130 | 32832766 | 116.1 | 0.737 | 0.596 | 0.737 |
| rs3130388 | 29958916 | 103.2 | 0.737 | 0.531 | 0.737 |
| rs1061815 | 29684132 | 106.2 | 0.736 | 0.667 | 0.736 |
| rs241455 | 32566776 | 102.2 | 0.734 | 0.599 | 0.734 |
| rs3891288 | 29141612 | 111.2 | 0.734 | 0.558 | 0.734 |
| rs1655938 | 29665973 | 111.2 | 0.733 | 0.693 | 0.733 |
| rs3131102 | 28954391 | 103.2 | 0.733 | 0.609 | 0.733 |
| rs6457566 | 32385481 | 116.1 | 0.731 | 0.729 | 0.731 |
| rs204887 | 31799983 | 111.2 | 0.731 | 0.228 | 0.731 |
| rs2844725 | 30235930 | 100.2 | 0.73 | 0.567 | 0.73 |
| rs2074491 | 31299204 | 98.2 | 0.729 | 0.648 | 0.729 |
| rs1233425 | 29566354 | 100.2 | 0.728 | 0.608 | 0.728 |
| rs4569 | 31696492 | 110.1 | 0.728 | 0.577 | 0.728 |
| rs2072109 | 30131031 | 98.2 | 0.728 | 0.515 | 0.728 |
| rs3130216 | 33070256 | 116.2 | 0.728 | 0.301 | 0.728 |
| rs3757340 | 30982850 | 107.1 | 0.724 | 0.684 | 0.724 |
| rs3116838 | 29140700 | 103.2 | 0.723 | 0.69 | 0.723 |
| rs7451419 | 32369487 | 116.1 | 0.72 | 0.667 | 0.72 |
| rs1475865 | 31428171 | 113.2 | 0.72 | 0.635 | 0.72 |
| rs1264315 | 30925871 | 113.1 | 0.72 | 0.605 | 0.72 |
| rs2269610 | 33282638 | 100.2 | 0.718 | 0.696 | 0.718 |
| rs3129012 | 29759400 | 103.2 | 0.718 | 0.416 | 0.718 |
| rs3132935 | 32190828 | 103.2 | 0.717 | 0.298 | 0.717 |
| rs2074473 | 30214943 | 98.2 | 0.715 | 0.693 | 0.715 |
| rs3130553 | 31143091 | 103.2 | 0.714 | 0.566 | 0.714 |
| rs2534672 | 31236316 | 110.2 | 0.713 | 0.67 | 0.713 |
| rs1573294 | 30447130 | 113.1 | 0.71 | 0.605 | 0.71 |
| rs7773534 | 29403950 | 116.1 | 0.71 | 0.495 | 0.71 |
| rs1737079 | 29495503 | 89.2 | 0.706 | 0.668 | 0.706 |
| rs805297 | 31681289 | 110.2 | 0.706 | 0.336 | 0.706 |
| rs805298 | 31679930 | 100.2 | 0.705 | 0.617 | 0.705 |
| rs3749951 | 31740262 | 107.2 | 0.703 | 0.631 | 0.703 |
| rs3094208 | 31151475 | 110.2 | 0.703 | 0.482 | 0.703 |
| rs805263 | 31686948 | 100.2 | 0.702 | 0.689 | 0.702 |
| rs3135330 | 32708806 | 103.2 | 0.701 | 0.542 | 0.701 |
| rs734181 | 33131655 | 111.2 | 0.7 | 0.454 | 0.7 |
| rs1050673 | 33163108 | 106.2 | 0.697 | 0.566 | 0.697 |
| rs3129948 | 32125401 | 103.2 | 0.696 | 0.1 | 0.696 |
| rs2517436 | 30714297 | 100.2 | 0.694 | 0.484 | 0.694 |
| rs2242664 | 31610089 | 103.2 | 0.693 | 0.632 | 0.693 |
| rs7761801 | 29744995 | 116.1 | 0.692 | 0.652 | 0.692 |
| rs2077022 | 32913091 | 103.2 | 0.692 | 0.495 | 0.692 |
| rs7743662 | 32450789 | 116.1 | 0.691 | 0.689 | 0.691 |
| rs1264319 | 30633181 | 100.2 | 0.69 | 0.383 | 0.69 |
| rs204990 | 31932187 | 110.1 | 0.689 | 0.635 | 0.689 |
| rs2736158 | 31658985 | 113.2 | 0.689 | 0.616 | 0.689 |
| rs13409 | 31192947 | 110.2 | 0.688 | 0.615 | 0.688 |
| rs3130057 | 31563314 | 103.2 | 0.684 | 0.61 | 0.684 |
| rs2857127 | 32778390 | 100.2 | 0.68 | 0.39 | 0.68 |
| rs7887 | 31923536 | 108.2 | 0.679 | 0.475 | 0.679 |
| rs2535277 | 31137570 | 100.2 | 0.678 | 0.621 | 0.678 |
| rs2395296 | 32913278 | 113.2 | 0.676 | 0.53 | 0.676 |
| rs2294883 | 32138208 | 111.2 | 0.675 | 0.55 | 0.675 |
| rs3129681 | 29422381 | 103.2 | 0.675 | 0.485 | 0.675 |
| rs3130141 | 30202935 | 103.1 | 0.673 | 0.667 | 0.673 |
| rs2285042 | 31955651 | 100.2 | 0.672 | 0.545 | 0.672 |
| rs5027663 | 30145898 | 113.2 | 0.671 | 0.384 | 0.671 |
| rs2294478 | 33091637 | 100.2 | 0.669 | 0.431 | 0.669 |
| rs6457710 | 33030884 | 116.1 | 0.668 | 0.498 | 0.668 |
| rs204900 | 32082610 | 100.2 | 0.667 | 0.635 | 0.667 |
| rs5018660 | 32468775 | 113.2 | 0.667 | 0.302 | 0.667 |
| rs6906587 | 29778731 | 116.1 | 0.665 | 0.657 | 0.665 |
| rs2242653 | 31446523 | 98.2 | 0.664 | 0.66 | 0.664 |
| rs444921 | 31702935 | 96.2 | 0.664 | 0.63 | 0.664 |
| rs2187820 | 32156630 | 103.2 | 0.664 | 0.457 | 0.664 |
| rs2071281 | 32215023 | 98.2 | 0.663 | 0.554 | 0.663 |
| rs3116820 | 29214223 | 103.2 | 0.663 | 0.502 | 0.663 |
| rs241407 | 32633497 | 110.2 | 0.662 | 0.62 | 0.662 |
| rs6457443 | 31539301 | 116.1 | 0.662 | 0.536 | 0.662 |
| rs668558 | 31856575 | 86.2 | 0.662 | 0.449 | 0.662 |
| rs2516675 | 30201278 | 111.2 | 0.655 | 0.558 | 0.655 |
| rs909726 | 29490749 | 113.2 | 0.648 | 0.384 | 0.648 |
| rs4642463 | 32723349 | 111.2 | 0.647 | 0.607 | 0.647 |
| rs3020644 | 31665384 | 101.1 | 0.647 | 0.508 | 0.647 |
| rs2280801 | 31650902 | 100.2 | 0.647 | 0.488 | 0.647 |
| rs1736976 | 29836570 | 89.2 | 0.646 | 0.484 | 0.646 |
| rs1362065 | 29428675 | 88.2 | 0.645 | 0.615 | 0.645 |
| rs3094188 | 30913003 | 103.2 | 0.645 | 0.559 | 0.645 |
| rs7764777 | 29822668 | 116.1 | 0.641 | 0.179 | 0.641 |
| rs4959117 | 32616391 | 111.2 | 0.639 | 0.566 | 0.639 |
| rs2844621 | 31300845 | 100.2 | 0.639 | 0.349 | 0.639 |
| rs3099840 | 31201479 | 103.2 | 0.638 | 0.588 | 0.638 |
| rs2073525 | 33283528 | 103.2 | 0.634 | 0.479 | 0.634 |
| rs3094693 | 30189724 | 103.1 | 0.631 | 0.582 | 0.631 |
| rs7759272 | 29747502 | 116.1 | 0.624 | 0.506 | 0.624 |
| rs6911350 | 29336182 | 116.1 | 0.624 | 0.194 | 0.624 |
| rs2070600 | 31922200 | 98.1 | 0.621 | 0.616 | 0.621 |
| rs2269428 | 31800957 | 100.2 | 0.621 | 0.601 | 0.621 |
| rs2302399 | 31151972 | 111.2 | 0.619 | 0.59 | 0.619 |
| rs1633098 | 29475786 | 110.2 | 0.618 | 0.469 | 0.618 |
| rs555007 | 31621090 | 98.2 | 0.617 | 0.537 | 0.617 |
| rs2508042 | 29659075 | 100.2 | 0.617 | 0.487 | 0.617 |
| rs3130653 | 30593529 | 113.2 | 0.615 | 0.518 | 0.615 |
| rs1705003 | 33387405 | 89.1 | 0.614 | 0.595 | 0.614 |
| rs764554 | 30439273 | 111.2 | 0.611 | 0.226 | 0.611 |
| rs2532942 | 30953121 | 111.1 | 0.61 | 0.5 | 0.61 |
| rs213207 | 33242617 | 87.2 | 0.61 | 0.365 | 0.61 |
| rs2428494 | 31380949 | 110.2 | 0.608 | 0.353 | 0.608 |
| rs1799910 | 33132050 | 100.1 | 0.606 | 0.543 | 0.606 |
| rs2071480 | 32823380 | 110.2 | 0.605 | 0.291 | 0.605 |
| rs1079687 | 30379031 | 113.2 | 0.604 | 0.579 | 0.604 |
| rs970902 | 33134111 | 111.2 | 0.601 | 0.59 | 0.601 |
| rs6900438 | 33370660 | 116.1 | 0.601 | 0.414 | 0.601 |
| rs213213 | 33176445 | 87.2 | 0.599 | 0.494 | 0.599 |
| rs410909 | 30009548 | 101.1 | 0.592 | 0.379 | 0.592 |
| rs1052486 | 31669523 | 113.2 | 0.573 | 0.534 | 0.573 |
| rs1150736 | 30104120 | 100.2 | 0.546 | 0.473 | 0.546 |
| rs130076 | 31183305 | 106.2 | 0.525 | 0.492 | 0.525 |
| rs2395174 | 32430660 | 113.2 | 0.467 | 0.175 | 0.467 |
| rs2252223 | 31142279 | 100.2 | 0.403 | 0.299 | 0.403 |
| rs1264303 | 30943321 | 106.2 | 0.994 | 0.674 | 0.994 |
| rs2240061 | 31178673 | 98.2 | 0.993 | 0.679 | 0.993 |
| rs1345229 | 29953153 | 111.2 | 0.99 | 0.681 | 0.99 |
| rs2395185 | 32452654 | 111.2 | 0.99 | 0.645 | 0.99 |
| rs3130283 | 32158291 | 103.1 | 0.987 | 0.8 | 0.987 |
| rs2257914 | 30181335 | 100.2 | 0.984 | 0.643 | 0.984 |
| rs916920 | 30938010 | 113.1 | 0.984 | 0.597 | 0.984 |
| rs2249059 | 30717125 | 103.2 | 0.98 | 0.696 | 0.98 |
| rs2267640 | 30351651 | 100.2 | 0.975 | 0.88 | 0.975 |
| rs1150765 | 31188404 | 100.2 | 0.975 | 0.741 | 0.975 |
| rs3749967 | 32054601 | 107.2 | 0.973 | 0.832 | 0.973 |
| rs3817963 | 32138844 | 107.2 | 0.972 | 0.752 | 0.972 |
| rs2051549 | 32724874 | 94.2 | 0.972 | 0.654 | 0.972 |
| rs1894408 | 32780248 | 113.2 | 0.97 | 0.886 | 0.97 |
| rs3130315 | 31991442 | 111.2 | 0.969 | 0.839 | 0.969 |
| rs761187 | 32088793 | 113.2 | 0.969 | 0.797 | 0.969 |
| rs3749966 | 32032264 | 107.2 | 0.969 | 0.714 | 0.969 |
| rs2395153 | 32365104 | 113.2 | 0.968 | 0.743 | 0.968 |
| rs1150735 | 30105988 | 100.2 | 0.968 | 0.67 | 0.968 |
| rs7843 | 29923125 | 106.2 | 0.968 | 0.567 | 0.968 |
| rs2074478 | 31166460 | 103.2 | 0.967 | 0.866 | 0.967 |
| rs204993 | 31926338 | 96.1 | 0.967 | 0.836 | 0.967 |
| rs2301271 | 32719981 | 100.2 | 0.965 | 0.812 | 0.965 |
| rs2284168 | 30233395 | 100.2 | 0.961 | 0.738 | 0.961 |
| rs11244 | 32551481 | 106.2 | 0.955 | 0.838 | 0.955 |
| rs1264583 | 30354186 | 100.2 | 0.954 | 0.867 | 0.954 |
| rs2074474 | 30370583 | 98.2 | 0.953 | 0.602 | 0.953 |
| rs2213571 | 32714725 | 113.2 | 0.944 | 0.888 | 0.944 |
| rs204991 | 31932123 | 110.1 | 0.943 | 0.824 | 0.943 |
| rs3129881 | 32180241 | 103.2 | 0.943 | 0.784 | 0.943 |
| rs1049623 | 30925642 | 110.1 | 0.929 | 0.772 | 0.929 |
| rs974962 | 30085365 | 113.2 | 0.921 | 0.52 | 0.921 |
| rs8512 | 30772491 | 111.2 | 0.919 | 0.834 | 0.919 |
| rs2071287 | 32190187 | 103.2 | 0.916 | 0.794 | 0.916 |
| rs1058318 | 30572879 | 106.2 | 0.91 | 0.811 | 0.91 |
| rs2269702 | 30445893 | 100.2 | 0.888 | 0.823 | 0.888 |
| rs2286404 | 30034021 | 113.2 | 0.862 | 0.724 | 0.862 |
| rs2269707 | 30705253 | 100.2 | 0.836 | 0.784 | 0.836 |
| rs2269424 | 32158239 | 100.2 | 0.813 | 0.77 | 0.813 |
| rs2040409 | 32608472 | 94.2 | 0.987 | 0.584 | 0.987 |
| rs241424 | 32806454 | 100.2 | 0.984 | 0.846 | 0.984 |
| rs2854275 | 32622585 | 100.2 | 0.982 | 0.75 | 0.982 |
| rs3880457 | 31690977 | 113.1 | 0.964 | 0.667 | 0.964 |
| rs4711206 | 29757942 | 111.2 | 0.999 | 0.728 | 0.999 |
| rs1233495 | 29214876 | 100.2 | 0.999 | 0.629 | 0.999 |
| rs3130055 | 31556298 | 103.2 | 0.997 | 0.841 | 0.997 |
| rs2071593 | 31571678 | 96.2 | 0.997 | 0.774 | 0.997 |
| rs7758397 | 32489119 | 116.1 | 0.997 | 0.727 | 0.997 |
| rs3130453 | 31188667 | 106.2 | 0.997 | 0.653 | 0.997 |
| rs2242657 | 31661170 | 98.2 | 0.996 | 0.68 | 0.996 |
| rs3130605 | 32978678 | 111.2 | 0.996 | 0.629 | 0.996 |
| rs3093559 | 31606473 | 103.2 | 0.995 | 0.802 | 0.995 |
| rs2235498 | 33131877 | 98.2 | 0.995 | 0.8 | 0.995 |
| rs3135034 | 32722419 | 103.2 | 0.995 | 0.701 | 0.995 |
| rs3129952 | 32385521 | 103.2 | 0.995 | 0.689 | 0.995 |
| rs6457602 | 32549256 | 116.1 | 0.995 | 0.669 | 0.995 |
| rs2523451 | 31139909 | 113.2 | 0.995 | 0.668 | 0.995 |
| rs497239 | 31679519 | 100.2 | 0.994 | 0.73 | 0.994 |
| rs4959050 | 30187589 | 111.2 | 0.994 | 0.715 | 0.994 |
| rs714289 | 32899220 | 113.2 | 0.994 | 0.668 | 0.994 |
| rs447250 | 32982668 | 80.2 | 0.994 | 0.576 | 0.994 |
| rs2844775 | 29950180 | 100.2 | 0.993 | 0.689 | 0.993 |
| rs3135002 | 32439196 | 108.2 | 0.993 | 0.645 | 0.993 |
| rs3077 | 33034469 | 86.2 | 0.993 | 0.644 | 0.993 |
| rs3094205 | 30862620 | 110.2 | 0.993 | 0.634 | 0.993 |
| rs6927088 | 32045753 | 116.1 | 0.992 | 0.805 | 0.992 |
| rs2857174 | 32525525 | 100.2 | 0.992 | 0.794 | 0.992 |
| rs181997 | 32902199 | 92.1 | 0.992 | 0.761 | 0.992 |
| rs261950 | 30040319 | 89.2 | 0.992 | 0.716 | 0.992 |
| rs3117329 | 29290196 | 103.2 | 0.992 | 0.689 | 0.992 |
| rs3128931 | 32973155 | 103.2 | 0.992 | 0.688 | 0.992 |
| rs6904456 | 29459977 | 116.1 | 0.991 | 0.777 | 0.991 |
| rs4713216 | 29426584 | 111.2 | 0.991 | 0.735 | 0.991 |
| rs3097671 | 33040676 | 108.2 | 0.99 | 0.754 | 0.99 |
| rs3135368 | 32158035 | 103.2 | 0.989 | 0.742 | 0.989 |
| rs1264323 | 30626665 | 100.2 | 0.989 | 0.622 | 0.989 |
| rs3094073 | 30001982 | 103.2 | 0.988 | 0.808 | 0.988 |
| rs2523995 | 30163382 | 100.2 | 0.988 | 0.666 | 0.988 |
| rs3131635 | 31534870 | 103.2 | 0.987 | 0.82 | 0.987 |
| rs1610724 | 29826673 | 89.2 | 0.987 | 0.793 | 0.987 |
| rs4713337 | 30590589 | 111.2 | 0.987 | 0.734 | 0.987 |
| rs2855442 | 33130126 | 108.2 | 0.987 | 0.725 | 0.987 |
| rs885915 | 29973473 | 100.2 | 0.987 | 0.721 | 0.987 |
| rs241440 | 32568118 | 110.2 | 0.986 | 0.834 | 0.986 |
| rs879882 | 30910210 | 103.1 | 0.986 | 0.747 | 0.986 |
| rs6937061 | 33081259 | 116.1 | 0.986 | 0.73 | 0.986 |
| rs2023372 | 30142776 | 94.2 | 0.986 | 0.713 | 0.986 |
| rs1610601 | 29470941 | 89.2 | 0.986 | 0.644 | 0.986 |
| rs29228 | 29686182 | 103.2 | 0.985 | 0.662 | 0.985 |
| rs7454372 | 29496723 | 116.1 | 0.985 | 0.6 | 0.985 |
| rs707937 | 31789665 | 100.2 | 0.985 | 0.571 | 0.985 |
| rs7341251 | 30976303 | 116.1 | 0.984 | 0.815 | 0.984 |
| rs1800750 | 31601644 | 113.2 | 0.984 | 0.773 | 0.984 |
| rs2394518 | 29137277 | 113.2 | 0.984 | 0.721 | 0.984 |
| rs2301225 | 33037307 | 100.2 | 0.984 | 0.705 | 0.984 |
| rs1264459 | 30516651 | 110.2 | 0.983 | 0.88 | 0.983 |
| rs7773955 | 32708285 | 116.1 | 0.983 | 0.766 | 0.983 |
| rs15912 | 32937519 | 103.2 | 0.983 | 0.702 | 0.983 |
| rs1419675 | 29863465 | 100.1 | 0.983 | 0.645 | 0.983 |
| rs7450074 | 29199772 | 116.1 | 0.982 | 0.754 | 0.982 |
| rs437179 | 31699772 | 98.2 | 0.982 | 0.733 | 0.982 |
| rs3132486 | 31305485 | 103.2 | 0.982 | 0.605 | 0.982 |
| rs2071286 | 31950653 | 111.2 | 0.982 | 0.594 | 0.982 |
| rs2395029 | 31202538 | 100.2 | 0.981 | 0.866 | 0.981 |
| rs7765791 | 29472518 | 116.1 | 0.981 | 0.863 | 0.981 |
| rs3094576 | 29578695 | 103.2 | 0.981 | 0.71 | 0.981 |
| rs3819269 | 31149774 | 107.2 | 0.981 | 0.706 | 0.981 |
| rs2074471 | 30217980 | 98.1 | 0.981 | 0.702 | 0.981 |
| rs6929716 | 32778676 | 116.1 | 0.981 | 0.685 | 0.981 |
| rs2523608 | 31384313 | 110.2 | 0.981 | 0.675 | 0.981 |
| rs1264540 | 30448606 | 100.1 | 0.981 | 0.646 | 0.981 |
| rs3116996 | 33090635 | 103.2 | 0.981 | 0.622 | 0.981 |
| rs3130933 | 31192892 | 110.2 | 0.981 | 0.578 | 0.981 |
| rs6940867 | 31136181 | 116.1 | 0.98 | 0.696 | 0.98 |
| rs2535250 | 29405175 | 100.2 | 0.979 | 0.773 | 0.979 |
| rs6903608 | 32454025 | 116.1 | 0.979 | 0.763 | 0.979 |
| rs3117435 | 29380746 | 103.2 | 0.979 | 0.663 | 0.979 |
| rs1264425 | 30629379 | 100.2 | 0.979 | 0.567 | 0.979 |
| rs3873342 | 30728524 | 113.2 | 0.978 | 0.674 | 0.978 |
| rs1265059 | 31139301 | 100.2 | 0.977 | 0.847 | 0.977 |
| rs6911634 | 30086079 | 116.1 | 0.977 | 0.683 | 0.977 |
| rs464921 | 33233201 | 87.2 | 0.977 | 0.661 | 0.977 |
| rs4713221 | 29138497 | 111.2 | 0.977 | 0.54 | 0.977 |
| rs362544 | 29568144 | 113.2 | 0.976 | 0.852 | 0.976 |
| rs3130977 | 31142795 | 110.2 | 0.976 | 0.787 | 0.976 |
| rs2395352 | 33102179 | 100.2 | 0.976 | 0.715 | 0.976 |
| rs984802 | 30092119 | 113.2 | 0.976 | 0.693 | 0.976 |
| rs1049633 | 30928692 | 106.2 | 0.976 | 0.688 | 0.976 |
| rs6923005 | 29038620 | 116.1 | 0.975 | 0.783 | 0.975 |
| rs2001181 | 31299318 | 113.2 | 0.975 | 0.68 | 0.975 |
| rs2071541 | 32806271 | 110.2 | 0.974 | 0.842 | 0.974 |
| rs3807039 | 31849130 | 107.1 | 0.974 | 0.784 | 0.974 |
| rs6918223 | 32724098 | 116.1 | 0.973 | 0.809 | 0.973 |
| rs3130501 | 31200285 | 103.1 | 0.973 | 0.764 | 0.973 |
| rs3869092 | 30705209 | 113.1 | 0.973 | 0.746 | 0.973 |
| rs1736920 | 29567931 | 110.2 | 0.972 | 0.87 | 0.972 |
| rs3823418 | 31161768 | 107.2 | 0.972 | 0.87 | 0.972 |
| rs2844802 | 30009192 | 113.1 | 0.972 | 0.865 | 0.972 |
| rs2394412 | 30842972 | 113.2 | 0.972 | 0.753 | 0.972 |
| rs439844 | 31843697 | 80.1 | 0.971 | 0.769 | 0.971 |
| rs3763309 | 32146730 | 107.2 | 0.971 | 0.669 | 0.971 |
| rs7383312 | 31527437 | 116.1 | 0.97 | 0.865 | 0.97 |
| rs1633021 | 29517627 | 89.2 | 0.97 | 0.863 | 0.97 |
| rs1367727 | 32935891 | 103.2 | 0.97 | 0.666 | 0.97 |
| rs2269423 | 32171709 | 103.1 | 0.969 | 0.79 | 0.969 |
| rs29269 | 29388505 | 113.2 | 0.969 | 0.777 | 0.969 |
| rs2894054 | 30643032 | 110.1 | 0.969 | 0.774 | 0.969 |
| rs2534674 | 31235861 | 110.2 | 0.969 | 0.717 | 0.969 |
| rs6915177 | 29585044 | 116.1 | 0.969 | 0.712 | 0.969 |
| rs4713559 | 32610062 | 111.2 | 0.969 | 0.607 | 0.969 |
| rs7382836 | 32735881 | 116.1 | 0.968 | 0.873 | 0.968 |
| rs4071986 | 30190589 | 113.1 | 0.968 | 0.836 | 0.968 |
| rs735883 | 32809467 | 113.1 | 0.968 | 0.789 | 0.968 |
| rs3793127 | 32142672 | 107.1 | 0.968 | 0.781 | 0.968 |
| rs2301269 | 32914840 | 100.2 | 0.968 | 0.751 | 0.968 |
| rs2844685 | 30713841 | 100.2 | 0.967 | 0.768 | 0.967 |
| rs6908943 | 32636268 | 116.1 | 0.967 | 0.731 | 0.967 |
| rs7383578 | 29834966 | 116.1 | 0.967 | 0.689 | 0.967 |
| rs1633088 | 29776103 | 98.2 | 0.967 | 0.587 | 0.967 |
| rs3871247 | 30916460 | 113.2 | 0.966 | 0.82 | 0.966 |
| rs461338 | 33219628 | 106.2 | 0.966 | 0.807 | 0.966 |
| rs3807031 | 30094673 | 111.2 | 0.965 | 0.781 | 0.965 |
| rs6911709 | 29566381 | 116.1 | 0.965 | 0.77 | 0.965 |
| rs2524005 | 29960037 | 100.2 | 0.965 | 0.766 | 0.965 |
| rs1736991 | 29829497 | 89.2 | 0.964 | 0.648 | 0.964 |
| rs4713422 | 30770660 | 111.2 | 0.963 | 0.786 | 0.963 |
| rs6921921 | 29817970 | 116.1 | 0.963 | 0.73 | 0.963 |
| rs7453137 | 28930305 | 116.1 | 0.963 | 0.713 | 0.963 |
| rs1801823 | 33261030 | 89.2 | 0.963 | 0.657 | 0.963 |
| rs498240 | 31663350 | 110.1 | 0.962 | 0.906 | 0.962 |
| rs6906730 | 32405178 | 116.1 | 0.962 | 0.877 | 0.962 |
| rs2057726 | 30106108 | 113.2 | 0.962 | 0.849 | 0.962 |
| rs2523989 | 30139461 | 100.2 | 0.962 | 0.804 | 0.962 |
| rs2071788 | 28974859 | 96.2 | 0.962 | 0.776 | 0.962 |
| rs4713214 | 29425994 | 111.2 | 0.961 | 0.794 | 0.961 |
| rs29273 | 29381747 | 102.2 | 0.961 | 0.75 | 0.961 |
| rs2517912 | 29454702 | 111.2 | 0.961 | 0.455 | 0.961 |
| rs1265156 | 30913055 | 100.2 | 0.96 | 0.6 | 0.96 |
| rs4386816 | 31306426 | 111.2 | 0.96 | 0.563 | 0.96 |
| rs2075702 | 31708269 | 98.1 | 0.959 | 0.805 | 0.959 |
| rs1264616 | 30035065 | 113.2 | 0.959 | 0.78 | 0.959 |
| rs1419633 | 29143968 | 113.2 | 0.959 | 0.773 | 0.959 |
| rs389883 | 31718218 | 100.2 | 0.959 | 0.721 | 0.959 |
| rs2523921 | 30745848 | 100.1 | 0.958 | 0.895 | 0.958 |
| rs209474 | 32917999 | 113.2 | 0.958 | 0.89 | 0.958 |
| rs2532921 | 30693328 | 100.1 | 0.958 | 0.753 | 0.958 |
| rs2207338 | 28970579 | 113.2 | 0.958 | 0.729 | 0.958 |
| rs2844829 | 29580140 | 113.2 | 0.958 | 0.666 | 0.958 |
| rs1035798 | 31921979 | 113.1 | 0.958 | 0.633 | 0.958 |
| rs2857597 | 31643683 | 100.2 | 0.957 | 0.775 | 0.957 |
| rs2747421 | 29707637 | 110.2 | 0.957 | 0.748 | 0.957 |
| rs2246618 | 31537720 | 100.2 | 0.957 | 0.668 | 0.957 |
| rs2855812 | 31531624 | 111.2 | 0.957 | 0.619 | 0.957 |
| rs6457259 | 30727325 | 116.1 | 0.956 | 0.918 | 0.956 |
| rs2071799 | 32484932 | 98.2 | 0.956 | 0.855 | 0.956 |
| rs7754054 | 29454928 | 116.1 | 0.956 | 0.838 | 0.956 |
| rs1049526 | 32719561 | 106.2 | 0.956 | 0.837 | 0.956 |
| rs2284191 | 32970036 | 110.2 | 0.956 | 0.72 | 0.956 |
| rs1655944 | 29659154 | 89.2 | 0.956 | 0.716 | 0.956 |
| rs7451258 | 31235410 | 116.1 | 0.956 | 0.543 | 0.956 |
| rs2844665 | 30777613 | 100.2 | 0.955 | 0.764 | 0.955 |
| rs3763364 | 32809041 | 107.2 | 0.955 | 0.523 | 0.955 |
| rs3128982 | 31187949 | 103.2 | 0.954 | 0.785 | 0.954 |
| rs6457358 | 31299307 | 116.1 | 0.954 | 0.773 | 0.954 |
| rs7758512 | 30030989 | 116.1 | 0.954 | 0.771 | 0.954 |
| rs3115673 | 31868723 | 103.2 | 0.954 | 0.72 | 0.954 |
| rs7029 | 31688637 | 106.2 | 0.954 | 0.661 | 0.954 |
| rs1041885 | 32432292 | 113.2 | 0.954 | 0.594 | 0.954 |
| rs259939 | 29782339 | 110.2 | 0.953 | 0.787 | 0.953 |
| rs3093993 | 31549468 | 103.2 | 0.953 | 0.645 | 0.953 |
| rs6457595 | 32469792 | 116.1 | 0.952 | 0.896 | 0.952 |
| rs3128844 | 29521688 | 103.2 | 0.952 | 0.742 | 0.952 |
| rs3094212 | 31149536 | 110.2 | 0.952 | 0.72 | 0.952 |
| rs1264695 | 30129079 | 100.2 | 0.952 | 0.559 | 0.952 |
| rs2516393 | 31565471 | 110.2 | 0.951 | 0.908 | 0.951 |
| rs209146 | 28930948 | 92.2 | 0.951 | 0.878 | 0.951 |
| rs4713243 | 29491719 | 111.2 | 0.95 | 0.742 | 0.95 |
| rs532385 | 32215098 | 83.2 | 0.949 | 0.516 | 0.949 |
| rs1610628 | 29498967 | 89.2 | 0.948 | 0.898 | 0.948 |
| rs8111 | 31853932 | 108.2 | 0.948 | 0.798 | 0.948 |
| rs2242660 | 31656433 | 100.2 | 0.948 | 0.787 | 0.948 |
| rs3115592 | 29923350 | 103.2 | 0.948 | 0.742 | 0.948 |
| rs3135029 | 32915188 | 103.2 | 0.948 | 0.449 | 0.948 |
| rs3869109 | 30954954 | 111.2 | 0.947 | 0.876 | 0.947 |
| rs2066951 | 29516917 | 113.1 | 0.947 | 0.793 | 0.947 |
| rs2282851 | 33281756 | 103.2 | 0.947 | 0.777 | 0.947 |
| rs2517713 | 29978499 | 100.1 | 0.947 | 0.641 | 0.947 |
| rs7770505 | 30089707 | 116.1 | 0.945 | 0.722 | 0.945 |
| rs3117294 | 29424087 | 103.2 | 0.945 | 0.662 | 0.945 |
| rs2857218 | 32626097 | 100.2 | 0.944 | 0.837 | 0.944 |
| rs2242656 | 31384860 | 100.2 | 0.943 | 0.891 | 0.943 |
| rs2275854 | 29747596 | 113.2 | 0.943 | 0.871 | 0.943 |
| rs3130050 | 31677600 | 103.2 | 0.943 | 0.798 | 0.943 |
| rs7745413 | 29975869 | 116.1 | 0.943 | 0.771 | 0.943 |
| rs2894066 | 28956479 | 111.2 | 0.943 | 0.55 | 0.943 |
| rs3213469 | 31859611 | 106.2 | 0.942 | 0.746 | 0.942 |
| rs406936 | 31991915 | 101.1 | 0.942 | 0.661 | 0.942 |
| rs3916765 | 32456307 | 108.2 | 0.94 | 0.805 | 0.94 |
| rs3873257 | 29584167 | 113.2 | 0.938 | 0.902 | 0.938 |
| rs453658 | 29290606 | 111.2 | 0.938 | 0.785 | 0.938 |
| rs660594 | 31608008 | 110.1 | 0.937 | 0.914 | 0.937 |
| rs1008403 | 29903585 | 86.2 | 0.937 | 0.734 | 0.937 |
| rs763009 | 28977660 | 113.2 | 0.936 | 0.931 | 0.936 |
| rs3130649 | 30574012 | 110.1 | 0.936 | 0.864 | 0.936 |
| rs7752270 | 29116542 | 116.1 | 0.936 | 0.817 | 0.936 |
| rs659445 | 31923123 | 110.2 | 0.936 | 0.776 | 0.936 |
| rs7753474 | 29403894 | 116.1 | 0.935 | 0.858 | 0.935 |
| rs3130762 | 29140336 | 103.2 | 0.934 | 0.84 | 0.934 |
| rs3094127 | 30758579 | 103.1 | 0.934 | 0.754 | 0.934 |
| rs7754598 | 30528428 | 116.1 | 0.933 | 0.823 | 0.933 |
| rs7774255 | 29336926 | 116.1 | 0.933 | 0.782 | 0.933 |
| rs465506 | 33360460 | 80.2 | 0.933 | 0.695 | 0.933 |
| rs2853961 | 31291281 | 100.2 | 0.933 | 0.484 | 0.933 |
| rs3815101 | 30575923 | 107.2 | 0.931 | 0.7 | 0.931 |
| rs2517892 | 29864045 | 100.2 | 0.93 | 0.649 | 0.93 |
| rs2187689 | 32623404 | 113.2 | 0.929 | 0.895 | 0.929 |
| rs6901092 | 33090422 | 116.1 | 0.929 | 0.894 | 0.929 |
| rs2857605 | 31295609 | 100.2 | 0.929 | 0.868 | 0.929 |
| rs2516676 | 30201240 | 100.2 | 0.928 | 0.808 | 0.928 |
| rs6922756 | 31225239 | 116.1 | 0.926 | 0.901 | 0.926 |
| rs2074475 | 29908648 | 96.1 | 0.926 | 0.584 | 0.926 |
| rs1264432 | 30623138 | 101.2 | 0.924 | 0.71 | 0.924 |
| rs2267633 | 29341599 | 102.2 | 0.922 | 0.878 | 0.922 |
| rs2517819 | 29919494 | 100.2 | 0.922 | 0.735 | 0.922 |
| rs3130637 | 31546883 | 103.2 | 0.917 | 0.889 | 0.917 |
| rs3116837 | 28955415 | 103.2 | 0.917 | 0.679 | 0.917 |
| rs3793125 | 31737688 | 107.2 | 0.916 | 0.776 | 0.916 |
| rs757257 | 29913216 | 108.2 | 0.911 | 0.707 | 0.911 |
| rs3117038 | 33078904 | 103.2 | 0.909 | 0.793 | 0.909 |
| rs2523589 | 31386083 | 100.2 | 0.905 | 0.805 | 0.905 |
| rs2747479 | 33338901 | 111.2 | 0.905 | 0.698 | 0.905 |
| rs3130244 | 30614055 | 103.1 | 0.903 | 0.77 | 0.903 |
| rs7357041 | 29387670 | 116.1 | 0.902 | 0.88 | 0.902 |
| rs4713504 | 31870967 | 111.2 | 0.902 | 0.788 | 0.902 |
| rs2187580 | 33180406 | 96.2 | 0.901 | 0.781 | 0.901 |
| rs3132945 | 32009437 | 103.2 | 0.899 | 0.834 | 0.899 |
| rs3998751 | 29855059 | 108.2 | 0.898 | 0.781 | 0.898 |
| rs2860580 | 29967091 | 101.2 | 0.898 | 0.75 | 0.898 |
| rs7757916 | 32567554 | 116.1 | 0.897 | 0.446 | 0.897 |
| rs3130014 | 33305001 | 103.2 | 0.896 | 0.867 | 0.896 |
| rs3830076 | 31867001 | 107.1 | 0.894 | 0.882 | 0.894 |
| rs2158280 | 29120082 | 110.2 | 0.893 | 0.775 | 0.893 |
| rs1233391 | 29618492 | 100.1 | 0.892 | 0.782 | 0.892 |
| rs2844477 | 31637455 | 100.2 | 0.891 | 0.463 | 0.891 |
| rs7453097 | 31593947 | 116.1 | 0.89 | 0.499 | 0.89 |
| rs3130816 | 28974666 | 103.2 | 0.889 | 0.888 | 0.889 |
| rs4151648 | 31683833 | 110.1 | 0.889 | 0.445 | 0.889 |
| rs3131090 | 29140226 | 103.2 | 0.888 | 0.851 | 0.888 |
| rs1063318 | 32403502 | 106.2 | 0.886 | 0.371 | 0.886 |
| rs3130660 | 30767083 | 103.2 | 0.883 | 0.878 | 0.883 |
| rs1264549 | 30438574 | 100.2 | 0.881 | 0.746 | 0.881 |
| rs2299851 | 31489360 | 100.2 | 0.881 | 0.724 | 0.881 |
| rs2844682 | 30716906 | 111.1 | 0.88 | 0.865 | 0.88 |
| rs2051625 | 33279884 | 94.2 | 0.879 | 0.805 | 0.879 |
| rs2178899 | 31377514 | 113.2 | 0.879 | 0.801 | 0.879 |
| rs3130389 | 29958957 | 103.2 | 0.875 | 0.641 | 0.875 |
| rs3749981 | 32633439 | 107.2 | 0.874 | 0.5 | 0.874 |
| rs1233386 | 29620642 | 100.2 | 0.869 | 0.824 | 0.869 |
| rs2142234 | 31697814 | 110.2 | 0.865 | 0.748 | 0.865 |
| rs2747419 | 29710061 | 100.2 | 0.864 | 0.837 | 0.864 |
| rs356971 | 29750555 | 92.2 | 0.863 | 0.855 | 0.863 |
| rs1079541 | 30177514 | 113.2 | 0.863 | 0.669 | 0.863 |
| rs2273192 | 29408937 | 100.2 | 0.862 | 0.732 | 0.862 |
| rs1015466 | 29856674 | 100.2 | 0.862 | 0.464 | 0.862 |
| rs4947324 | 31298888 | 111.2 | 0.858 | 0.785 | 0.858 |
| rs2071295 | 31809457 | 98.2 | 0.852 | 0.734 | 0.852 |
| rs3130010 | 33311264 | 103.2 | 0.847 | 0.799 | 0.847 |
| rs2517424 | 30720754 | 100.2 | 0.845 | 0.745 | 0.845 |
| rs3093986 | 31552149 | 103.2 | 0.831 | 0.796 | 0.831 |
| rs2006752 | 29213864 | 113.2 | 0.828 | 0.736 | 0.828 |
| rs2530699 | 30723998 | 100.2 | 0.828 | 0.585 | 0.828 |
| rs707917 | 31428512 | 113.2 | 0.827 | 0.739 | 0.827 |
| rs6531 | 33164898 | 108.1 | 0.825 | 0.803 | 0.825 |
| rs2206042 | 29205028 | 110.2 | 0.825 | 0.725 | 0.825 |
| rs6457452 | 31854402 | 116.1 | 0.823 | 0.524 | 0.823 |
| rs1362064 | 29423356 | 88.2 | 0.816 | 0.653 | 0.816 |
| rs4424066 | 32125185 | 113.2 | 0.815 | 0.298 | 0.815 |
| rs3094110 | 30569801 | 103.2 | 0.814 | 0.808 | 0.814 |
| rs2242668 | 31544579 | 98.2 | 0.814 | 0.803 | 0.814 |
| rs6910451 | 29342724 | 116.1 | 0.814 | 0.753 | 0.814 |
| rs3823355 | 30002851 | 107.2 | 0.809 | 0.573 | 0.809 |
| rs3130667 | 30513999 | 103.2 | 0.807 | 0.614 | 0.807 |
| rs2071293 | 32088715 | 96.1 | 0.798 | 0.653 | 0.798 |
| rs1063478 | 32910958 | 111.2 | 0.797 | 0.796 | 0.797 |
| rs2242654 | 31425249 | 98.1 | 0.796 | 0.656 | 0.796 |
| rs2394994 | 31162376 | 100.2 | 0.793 | 0.747 | 0.793 |
| rs3130805 | 29077211 | 103.2 | 0.793 | 0.477 | 0.793 |
| rs2269429 | 31799940 | 100.2 | 0.793 | 0.301 | 0.793 |
| rs3132587 | 30745781 | 103.2 | 0.791 | 0.581 | 0.791 |
| rs2300825 | 32625659 | 100.2 | 0.789 | 0.698 | 0.789 |
| rs7744304 | 32451227 | 116.1 | 0.788 | 0.723 | 0.788 |
| rs1233388 | 29616179 | 100.1 | 0.787 | 0.648 | 0.787 |
| rs4711279 | 31861106 | 111.2 | 0.787 | 0.371 | 0.787 |
| rs6915800 | 31892654 | 116.1 | 0.777 | 0.757 | 0.777 |
| rs11908 | 32938171 | 106.2 | 0.772 | 0.737 | 0.772 |
| rs1059288 | 33260371 | 116.2 | 0.767 | 0.656 | 0.767 |
| rs3025657 | 29579073 | 102.2 | 0.766 | 0.714 | 0.766 |
| rs213209 | 33169673 | 116.2 | 0.762 | 0.678 | 0.762 |
| rs7766900 | 32831555 | 116.1 | 0.761 | 0.257 | 0.761 |
| rs3130268 | 33308852 | 116.2 | 0.756 | 0.663 | 0.756 |
| rs3749952 | 31741835 | 111.2 | 0.752 | 0.699 | 0.752 |
| rs6456951 | 29488632 | 116.1 | 0.738 | 0.632 | 0.738 |
| rs2523974 | 29860186 | 100.2 | 0.736 | 0.695 | 0.736 |
| rs6904236 | 30656841 | 116.1 | 0.728 | 0.645 | 0.728 |
| rs4713518 | 32028094 | 111.2 | 0.728 | 0.518 | 0.728 |
| rs2267646 | 32708744 | 100.2 | 0.721 | 0.684 | 0.721 |
| rs502626 | 32049023 | 108.2 | 0.697 | 0.643 | 0.697 |
| rs6468 | 31744340 | 86.2 | 0.675 | 0.376 | 0.675 |
| rs3129110 | 29147775 | 103.2 | 0.666 | 0.49 | 0.666 |
| rs6456947 | 29450733 | 116.1 | 0.666 | 0.486 | 0.666 |
| rs3129154 | 29202338 | 103.2 | 0.658 | 0.633 | 0.658 |
| rs1009382 | 32045823 | 103.2 | 0.655 | 0.571 | 0.655 |
| rs1043618 | 31554265 | 111.1 | 0.652 | 0.625 | 0.652 |
| rs7754402 | 29391405 | 116.1 | 0.63 | 0.449 | 0.63 |
| rs2269706 | 30714010 | 100.2 | 0.617 | 0.612 | 0.617 |
| rs4713505 | 32124732 | 111.2 | 0.611 | 0.304 | 0.611 |
| rs205000 | 31906635 | 106.2 | 0.96 | 0.652 | 0.96 |
| rs2071278 | 32185187 | 103.2 | 0.935 | 0.91 | 0.935 |
| rs477005 | 32041257 | 87.2 | 0.929 | 0.802 | 0.929 |
| rs535586 | 31919326 | 100.1 | 0.961 | 0.618 | 0.961 |
| rs1052248 | 31615258 | 110.2 | 0.993 | 0.749 | 0.993 |
| rs621701 | 31961861 | 101.2 | 0.984 | 0.766 | 0.984 |
| rs2072679 | 31638197 | 98.1 | 0.98 | 0.758 | 0.98 |
| rs805257 | 31692878 | 100.2 | 0.978 | 0.68 | 0.978 |
| rs469064 | 33243171 | 94.1 | 0.955 | 0.906 | 0.955 |
| rs1264517 | 30181590 | 100.1 | 0.952 | 0.866 | 0.952 |
| rs211457 | 33367092 | 116.2 | 0.949 | 0.754 | 0.949 |
| rs537160 | 31687158 | 110.1 | 0.941 | 0.771 | 0.941 |
| rs2075800 | 31548704 | 100.2 | 0.921 | 0.827 | 0.921 |
| rs707926 | 31807660 | 100.2 | 0.904 | 0.784 | 0.904 |
| rs387608 | 31712315 | 100.1 | 0.866 | 0.764 | 0.866 |
| rs2071888 | 33274302 | 106.2 | 0.827 | 0.628 | 0.827 |
| rs2073524 | 33283250 | 103.2 | 0.816 | 0.725 | 0.816 |
| rs805306 | 31750845 | 100.2 | 0.75 | 0.615 | 0.75 |
| rs486416 | 31914889 | 87.1 | 0.738 | 0.574 | 0.738 |
| rs652888 | 31621992 | 100.1 | 0.709 | 0.588 | 0.709 |
| rs2075798 | 31905730 | 98.1 | 0.69 | 0.602 | 0.69 |

TABLE 2

| | Allele | Allele | Locas |
|---|---|---|---|
| | Sequence | sequence | sequence |
| SNP Name | of first ASO | of second ASO | of LSO |
| rs2395256 | SEQ ID NO: 1 | SEQ ID NO: 2367 | SEQ ID NO: 4733 |
| rs1894411 | SEQ ID NO: 2 | SEQ ID NO: 2368 | SEQ ID NO: 4734 |
| rs2621414 | SEQ ID NO: 3 | SEQ ID NO: 2369 | SEQ ID NO: 4735 |
| rs1264298 | SEQ ID NO: 4 | SEQ ID NO: 2370 | SEQ ID NO: 4736 |
| rs1264307 | SEQ ID NO: 5 | SEQ ID NO: 2371 | SEQ ID NO: 4737 |
| rs241454 | SEQ ID NO: 6 | SEQ ID NO: 2372 | SEQ ID NO: 4738 |
| rs3134996 | SEQ ID NO: 7 | SEQ ID NO: 2373 | SEQ ID NO: 4739 |
| rs2294882 | SEQ ID NO: 8 | SEQ ID NO: 2374 | SEQ ID NO: 4740 |
| rs2001100 | SEQ ID NO: 9 | SEQ ID NO: 2375 | SEQ ID NO: 4741 |
| rs2273017 | SEQ ID NO: 10 | SEQ ID NO: 2376 | SEQ ID NO: 4742 |
| rs3817969 | SEQ ID NO: 11 | SEQ ID NO: 2377 | SEQ ID NO: 4743 |
| rs761189 | SEQ ID NO: 12 | SEQ ID NO: 2378 | SEQ ID NO: 4744 |
| rs3129736 | SEQ ID NO: 13 | SEQ ID NO: 2379 | SEQ ID NO: 4745 |
| rs3763350 | SEQ ID NO: 14 | SEQ ID NO: 2380 | SEQ ID NO: 4746 |
| rs3135374 | SEQ ID NO: 15 | SEQ ID NO: 2381 | SEQ ID NO: 4747 |
| rs2294880 | SEQ ID NO: 16 | SEQ ID NO: 2382 | SEQ ID NO: 4748 |
| rs397081 | SEQ ID NO: 17 | SEQ ID NO: 2383 | SEQ ID NO: 4749 |
| rs3021302 | SEQ ID NO: 18 | SEQ ID NO: 2384 | SEQ ID NO: 4750 |
| rs3129871 | SEQ ID NO: 19 | SEQ ID NO: 2385 | SEQ ID NO: 4751 |
| rs365053 | SEQ ID NO: 20 | SEQ ID NO: 2386 | SEQ ID NO: 4752 |
| rs1264308 | SEQ ID NO: 21 | SEQ ID NO: 2387 | SEQ ID NO: 4753 |
| rs3806156 | SEQ ID NO: 22 | SEQ ID NO: 2388 | SEQ ID NO: 4754 |
| rs2253588 | SEQ ID NO: 23 | SEQ ID NO: 2389 | SEQ ID NO: 47SS |
| rs2074512 | SEQ ID NO: 24 | SEQ ID NO: 2390 | SEQ ID NO: 47S6 |
| rs2076536 | SEQ ID NO: 25 | SEQ ID NO: 2391 | SEQ ID NO: 47S7 |
| rs1988563 | SEQ ID NO: 26 | SEQ ID NO: 2392 | SEQ ID NO: 4758 |
| rs2253705 | SEQ ID NO: 27 | SEQ ID NO: 2393 | SEQ ID NO: 4759 |
| rs1894412 | SEQ ID NO: 28 | SEQ ID NO: 2394 | SEQ ID NO: 4760 |
| rs2071554 | SEQ ID NO: 29 | SEQ ID NO: 2395 | SEQ ID NO: 4761 |
| rs2395264 | SEQ ID NO: 30 | SEQ ID NO: 2396 | SEQ ID NO: 4762 |
| rs2071290 | SEQ ID NO: 31 | SEQ ID NO: 2397 | SEQ ID NO: 4763 |
| rs885912 | SEQ ID NO: 32 | SEQ ID NO: 2398 | SEQ ID NO: 4764 |
| rs2073717 | SEQ ID NO: 33 | SEQ ID NO: 2399 | SEQ ID NO: 4765 |
| rs3873444 | SEQ ID NO: 34 | SEQ ID NO: 2400 | SEQ ID NO: 4766 |
| rs261949 | SEQ ID NO: 35 | SEQ ID NO: 2401 | SEQ ID NO: 4767 |
| rs2187818 | SEQ ID NO: 36 | SEQ ID NO: 2402 | SEQ ID NO: 4768 |
| rs2071550 | SEQ ID NO: 37 | SEQ ID NO: 2403 | SEQ ID NO: 4769 |
| rs1573649 | SEQ ID NO: 38 | SEQ ID NO: 2404 | SEQ ID NO: 4770 |
| rs2395110 | SEQ ID NO: 39 | SEQ ID NO: 2405 | SEQ ID NO: 4771 |
| rs2073046 | SEQ ID NO: 40 | SEQ ID NO: 2406 | SEQ ID NO: 4772 |
| rs2073047 | SEQ ID NO: 41 | SEQ ID NO: 2407 | SEQ ID NO: 4773 |
| rs743862 | SEQ ID NO: 42 | SEQ ID NO: 2408 | SEQ ID NO: 4774 |
| rs2074488 | SEQ ID NO: 43 | SEQ ID NO: 2409 | SEQ ID NO: 4775 |
| rs2072895 | SEQ ID NO: 44 | SEQ ID NO: 2410 | SEQ ID NO: 4776 |
| rs2076537 | SEQ ID NO: 45 | SEQ ID NO: 2411 | SEQ ID NO: 4777 |
| rs2072110 | SEQ ID NO: 46 | SEQ ID NO: 2412 | SEQ ID NO: 4778 |
| rs13501 | SEQ ID NO: 47 | SEQ ID NO: 2413 | SEQ ID NO: 4779 |
| rs2072896 | SEQ ID NO: 48 | SEQ ID NO: 2414 | SEQ ID NO: 4780 |
| rs2581 | SEQ ID NO: 49 | SEQ ID NO: 2415 | SEQ ID NO: 4781 |
| rs2023472 | SEQ ID NO: 50 | SEQ ID NO: 2416 | SEQ ID NO: 4782 |
| rs2006165 | SEQ ID NO: 51 | SEQ ID NO: 2417 | SEQ ID NO: 4783 |
| rs2395533 | SEQ ID NO: 52 | SEQ ID NO: 2418 | SEQ ID NO: 4784 |
| rs2269703 | SEQ ID NO: 53 | SEQ ID NO: 2419 | SEQ ID NO: 4785 |
| rs259919 | SEQ ID NO: 54 | SEQ ID NO: 2420 | SEQ ID NO: 4786 |
| rs1265760 | SEQ ID NO: 55 | SEQ ID NO: 2421 | SEQ ID NO: 4787 |
| rs241428 | SEQ ID NO: 56 | SEQ ID NO: 2422 | SEQ ID NO: 4788 |
| rs3763316 | SEQ ID NO: 57 | SEQ ID NO: 2423 | SEQ ID NO: 4789 |
| rs8084 | SEQ ID NO: 58 | SEQ ID NO: 2424 | SEQ ID NO: 4790 |
| rs1042337 | SEQ ID NO: 59 | SEQ ID NO: 2425 | SEQ ID NO: 4791 |
| rs2269713 | SEQ ID NO: 60 | SEQ ID NO: 2426 | SEQ ID NO: 4792 |
| rs2239524 | SEQ ID NO: 61 | SEQ ID NO: 2427 | SEQ ID NO: 4793 |
| rs3115569 | SEQ ID NO: 62 | SEQ ID NO: 2428 | SEQ ID NO: 4794 |
| rs241448 | SEQ ID NO: 63 | SEQ ID NO: 2429 | SEQ ID NO: 4795 |
| rs2071475 | SEQ ID NO: 64 | SEQ ID NO: 2430 | SEQ ID NO: 4796 |
| rs365066 | SEQ ID NO: 65 | SEQ ID NO: 2431 | SEQ ID NO: 4797 |
| rs2022059 | SEQ ID NO: 66 | SEQ ID NO: 2432 | SEQ ID NO: 4798 |
| rs2071474 | SEQ ID NO: 67 | SEQ ID NO: 2433 | SEQ ID NO: 4799 |
| rs3129745 | SEQ ID NO: 68 | SEQ ID NO: 2434 | SEQ ID NO: 4800 |
| rs2213572 | SEQ ID NO: 69 | SEQ ID NO: 2435 | SEQ ID NO: 4801 |
| rs2286656 | SEQ ID NO: 70 | SEQ ID NO: 2436 | SEQ ID NO: 4802 |
| rs2070121 | SEQ ID NO: 71 | SEQ ID NO: 2437 | SEQ ID NO: 4803 |
| rs3117098 | SEQ ID NO: 72 | SEQ ID NO: 2438 | SEQ ID NO: 4804 |
| rs926594 | SEQ ID NO: 73 | SEQ ID NO: 2439 | SEQ ID NO: 4805 |
| rs2621393 | SEQ ID NO: 74 | SEQ ID NO: 2440 | SEQ ID NO: 4806 |
| rs7192 | SEQ ID NO: 75 | SEQ ID NO: 2441 | SEQ ID NO: 4807 |
| rs14004 | SEQ ID NO: 76 | SEQ ID NO: 2442 | SEQ ID NO: 4808 |
| rs3134943 | SEQ ID NO: 77 | SEQ ID NO: 2443 | SEQ ID NO: 4809 |
| rs2213585 | SEQ ID NO: 78 | SEQ ID NO: 2444 | SEQ ID NO: 4810 |
| rs2072894 | SEQ ID NO: 79 | SEQ ID NO: 2445 | SEQ ID NO: 4811 |
| rs2286655 | SEQ ID NO: 80 | SEQ ID NO: 2446 | SEQ ID NO: 4812 |
| rs886420 | SEQ ID NO: 81 | SEQ ID NO: 2447 | SEQ ID NO: 4813 |
| rs2074504 | SEQ ID NO: 82 | SEQ ID NO: 2448 | SEQ ID NO: 4814 |
| rs2301270 | SEQ ID NO: 83 | SEQ ID NO: 2449 | SEQ ID NO: 4815 |
| rs3763310 | SEQ ID NO: 84 | SEQ ID NO: 2450 | SEQ ID NO: 4816 |
| rs241446 | SEQ ID NO: 85 | SEQ ID NO: 2451 | SEQ ID NO: 4817 |
| rs3129876 | SEQ ID NO: 86 | SEQ ID NO: 2452 | SEQ ID NO: 4818 |
| rs1043483 | SEQ ID NO: 87 | SEQ ID NO: 2453 | SEQ ID NO: 4819 |
| rs2286405 | SEQ ID NO: 88 | SEQ ID NO: 2454 | SEQ ID NO: 4820 |
| rs2284175 | SEQ ID NO: 89 | SEQ ID NO: 2455 | SEQ ID NO: 4821 |
| rs1362122 | SEQ ID NO: 90 | SEQ ID NO: 2456 | SEQ ID NO: 4822 |
| rs1265155 | SEQ ID NO: 91 | SEQ ID NO: 2457 | SEQ ID NO: 4823 |
| rs2267641 | SEQ ID NO: 92 | SEQ ID NO: 2458 | SEQ ID NO: 4824 |
| rs2647089 | SEQ ID NO: 93 | SEQ ID NO: 2459 | SEQ ID NO: 4825 |
| rs2240619 | SEQ ID NO: 94 | SEQ ID NO: 2460 | SEQ ID NO: 4826 |
| rs1061808 | SEQ ID NO: 95 | SEQ ID NO: 2461 | SEQ ID NO: 4827 |
| rs2249099 | SEQ ID NO: 96 | SEQ ID NO: 2462 | SEQ ID NO: 4828 |
| rs1264309 | SEQ ID NO: 97 | SEQ ID NO: 2463 | SEQ ID NO: 4829 |
| rs3763305 | SEQ ID NO: 98 | SEQ ID NO: 2464 | SEQ ID NO: 4830 |
| rs2073724 | SEQ ID NO: 99 | SEQ ID NO: 2465 | SEQ ID NO: 4831 |
| rs2582 | SEQ ID NO: 100 | SEQ ID NO: 2466 | SEQ ID NO: 4832 |
| rs2074484 | SEQ ID NO: 101 | SEQ ID NO: 2467 | SEQ ID NO: 4833 |
| rs3134945 | SEQ ID NO: 102 | SEQ ID NO: 2468 | SEQ ID NO: 4834 |
| rs2269418 | SEQ ID NO: 103 | SEQ ID NO: 2469 | SEQ ID NO: 4835 |
| rs3749968 | SEQ ID NO: 104 | SEQ ID NO: 2470 | SEQ ID NO: 4836 |
| rs2856705 | SEQ ID NO: 105 | SEQ ID NO: 2471 | SEQ ID NO: 4837 |
| rs2071283 | SEQ ID NO: 106 | SEQ ID NO: 2472 | SEQ ID NO: 4838 |
| rs2239518 | SEQ ID NO: 107 | SEQ ID NO: 2473 | SEQ ID NO: 4839 |
| rs2240804 | SEQ ID NO: 108 | SEQ ID NO: 2474 | SEQ ID NO: 4840 |
| rs2076525 | SEQ ID NO: 109 | SEQ ID NO: 2475 | SEQ ID NO: 4841 |
| rs3806157 | SEQ ID NO: 110 | SEQ ID NO: 2476 | SEQ ID NO: 4842 |
| rs2187823 | SEQ ID NO: 111 | SEQ ID NO: 2477 | SEQ ID NO: 4843 |
| rs3129858 | SEQ ID NO: 112 | SEQ ID NO: 2478 | SEQ ID NO: 4844 |
| rs3135396 | SEQ ID NO: 113 | SEQ ID NO: 2479 | SEQ ID NO: 4845 |
| rs1736917 | SEQ ID NO: 114 | SEQ ID NO: 2480 | SEQ ID NO: 4846 |
| rs3213485 | SEQ ID NO: 115 | SEQ ID NO: 2481 | SEQ ID NO: 4847 |
| rs184003 | SEQ ID NO: 116 | SEQ ID NO: 2482 | SEQ ID NO: 4848 |
| rs2647086 | SEQ ID NO: 117 | SEQ ID NO: 2483 | SEQ ID NO: 4849 |
| rs2235383 | SEQ ID NO: 118 | SEQ ID NO: 2484 | SEQ ID NO: 4850 |
| rs3817970 | SEQ ID NO: 119 | SEQ ID NO: 2485 | SEQ ID NO: 4851 |
| rs2213574 | SEQ ID NO: 120 | SEQ ID NO: 2486 | SEQ ID NO: 4852 |
| rs2858857 | SEQ ID NO: 121 | SEQ ID NO: 2487 | SEQ ID NO: 4853 |
| rs2227139 | SEQ ID NO: 122 | SEQ ID NO: 2488 | SEQ ID NO: 4854 |
| rs709052 | SEQ ID NO: 123 | SEQ ID NO: 2489 | SEQ ID NO: 4855 |
| rs2239519 | SEQ ID NO: 124 | SEQ ID NO: 2490 | SEQ ID NO: 4856 |
| rs3844313 | SEQ ID NO: 125 | SEQ ID NO: 2491 | SEQ ID NO: 4857 |
| rs2076534 | SEQ ID NO: 126 | SEQ ID NO: 2492 | SEQ ID NO: 4858 |
| rs2074490 | SEQ ID NO: 127 | SEQ ID NO: 2493 | SEQ ID NO: 4859 |
| rs1049033 | SEQ ID NO: 128 | SEQ ID NO: 2494 | SEQ ID NO: 4860 |
| rs206019 | SEQ ID NO: 129 | SEQ ID NO: 2495 | SEQ ID NO: 4861 |
| rs1265157 | SEQ ID NO: 130 | SEQ ID NO: 2496 | SEQ ID NO: 4862 |
| rs1044043 | SEQ ID NO: 131 | SEQ ID NO: 2497 | SEQ ID NO: 4863 |
| rs1615839 | SEQ ID NO: 132 | SEQ ID NO: 2498 | SEQ ID NO: 4864 |
| rs2071555 | SEQ ID NO: 133 | SEQ ID NO: 2499 | SEQ ID NO: 4865 |
| rs241441 | SEQ ID NO: 134 | SEQ ID NO: 2500 | SEQ ID NO: 4866 |
| rs1624278 | SEQ ID NO: 135 | SEQ ID NO: 2501 | SEQ ID NO: 4867 |
| rs2395195 | SEQ ID NO: 136 | SEQ ID NO: 2502 | SEQ ID NO: 4868 |
| rs915667 | SEQ ID NO: 137 | SEQ ID NO: 2503 | SEQ ID NO: 4869 |
| rs1964995 | SEQ ID NO: 138 | SEQ ID NO: 2504 | SEQ ID NO: 4870 |
| rs3117226 | SEQ ID NO: 139 | SEQ ID NO: 2505 | SEQ ID NO: 4871 |
| rs2858333 | SEQ ID NO: 140 | SEQ ID NO: 2506 | SEQ ID NO: 4872 |
| rs3025644 | SEQ ID NO: 141 | SEQ ID NO: 2507 | SEQ ID NO: 4873 |
| rs3129939 | SEQ ID NO: 142 | SEQ ID NO: 2508 | SEQ ID NO: 4874 |
| rs2534655 | SEQ ID NO: 143 | SEQ ID NO: 2509 | SEQ ID NO: 4875 |
| rs2144425 | SEQ ID NO: 144 | SEQ ID NO: 2510 | SEQ ID NO: 4876 |
| rs2523969 | SEQ ID NO: 145 | SEQ ID NO: 2511 | SEQ ID NO: 4877 |
| rs2746149 | SEQ ID NO: 146 | SEQ ID NO: 2512 | SEQ ID NO: 4878 |
| rs1265048 | SEQ ID NO: 147 | SEQ ID NO: 2513 | SEQ ID NO: 4879 |
| rs2535276 | SEQ ID NO: 148 | SEQ ID NO: 2514 | SEQ ID NO: 4880 |
| rs3129267 | SEQ ID NO: 149 | SEQ ID NO: 2515 | SEQ ID NO: 4881 |
| rs3095314 | SEQ ID NO: 150 | SEQ ID NO: 2516 | SEQ ID NO: 4882 |
| rs2844720 | SEQ ID NO: 151 | SEQ ID NO: 2517 | SEQ ID NO: 4883 |
| rs2294476 | SEQ ID NO: 152 | SEQ ID NO: 2518 | SEQ ID NO: 4884 |
| rs2394517 | SEQ ID NO: 153 | SEQ ID NO: 2519 | SEQ ID NO: 4885 |
| rs916571 | SEQ ID NO: 154 | SEQ ID NO: 2520 | SEQ ID NO: 4886 |
| rs3129941 | SEQ ID NO: 155 | SEQ ID NO: 2521 | SEQ ID NO: 4887 |
| rs3817659 | SEQ ID NO: 156 | SEQ ID NO: 2522 | SEQ ID NO: 4888 |
| rs2596449 | SEQ ID NO: 157 | SEQ ID NO: 2523 | SEQ ID NO: 4889 |
| rs176250 | SEQ ID NO: 158 | SEQ ID NO: 2524 | SEQ ID NO: 4890 |
| rs6457116 | SEQ ID NO: 159 | SEQ ID NO: 2525 | SEQ ID NO: 4891 |
| rs2524070 | SEQ ID NO: 160 | SEQ ID NO: 2526 | SEQ ID NO: 4892 |
| rs2857178 | SEQ ID NO: 161 | SEQ ID NO: 2527 | SEQ ID NO: 4893 |
| rs3218822 | SEQ ID NO: 162 | SEQ ID NO: 2528 | SEQ ID NO: 4894 |
| rs3817981 | SEQ ID NO: 163 | SEQ ID NO: 2529 | SEQ ID NO: 4895 |
| rs1610735 | SEQ ID NO: 164 | SEQ ID NO: 2530 | SEQ ID NO: 4896 |
| rs2394737 | SEQ ID NO: 165 | SEQ ID NO: 2531 | SEQ ID NO: 4897 |
| rs2239530 | SEQ ID NO: 166 | SEQ ID NO: 2532 | SEQ ID NO: 4898 |
| rs4415152 | SEQ ID NO: 167 | SEQ ID NO: 2533 | SEQ ID NO: 4899 |
| rs4434496 | SEQ ID NO: 168 | SEQ ID NO: 2534 | SEQ ID NO: 4900 |
| rs3130602 | SEQ ID NO: 169 | SEQ ID NO: 2535 | SEQ ID NO: 4901 |
| rs211456 | SEQ ID NO: 170 | SEQ ID NO: 2536 | SEQ ID NO: 4902 |
| rs3091282 | SEQ ID NO: 171 | SEQ ID NO: 2537 | SEQ ID NO: 4903 |
| rs4959036 | SEQ ID NO: 172 | SEQ ID NO: 2538 | SEQ ID NO: 4904 |
| rs2524103 | SEQ ID NO: 173 | SEQ ID NO: 2539 | SEQ ID NO: 4905 |
| rs45855 | SEQ ID NO: 174 | SEQ ID NO: 2540 | SEQ ID NO: 4906 |
| rs3130404 | SEQ ID NO: 175 | SEQ ID NO: 2541 | SEQ ID NO: 4907 |
| rs211453 | SEQ ID NO: 176 | SEQ ID NO: 2542 | SEQ ID NO: 4908 |
| rs3135375 | SEQ ID NO: 177 | SEQ ID NO: 2543 | SEQ ID NO: 4909 |
| rs3129767 | SEQ ID NO: 178 | SEQ ID NO: 2544 | SEQ ID NO: 4910 |
| rs7744593 | SEQ ID NO: 179 | SEQ ID NO: 2545 | SEQ ID NO: 4911 |
| rs2734979 | SEQ ID NO: 180 | SEQ ID NO: 2546 | SEQ ID NO: 4912 |
| rs3094149 | SEQ ID NO: 181 | SEQ ID NO: 2547 | SEQ ID NO: 4913 |
| rs2517723 | SEQ ID NO: 182 | SEQ ID NO: 2548 | SEQ ID NO: 4914 |
| rs1632995 | SEQ ID NO: 183 | SEQ ID NO: 2549 | SEQ ID NO: 4915 |
| rs4248143 | SEQ ID NO: 184 | SEQ ID NO: 2550 | SEQ ID NO: 4916 |
| rs8807 | SEQ ID NO: 185 | SEQ ID NO: 2551 | SEQ ID NO: 4917 |
| rs3130745 | SEQ ID NO: 186 | SEQ ID NO: 2552 | SEQ ID NO: 4918 |
| rs2858331 | SEQ ID NO: 187 | SEQ ID NO: 2553 | SEQ ID NO: 4919 |
| rs2596503 | SEQ ID NO: 188 | SEQ ID NO: 2554 | SEQ ID NO: 4920 |
| rs2517529 | SEQ ID NO: 189 | SEQ ID NO: 2555 | SEQ ID NO: 4921 |
| rs761188 | SEQ ID NO: 190 | SEQ ID NO: 2556 | SEQ ID NO: 4922 |
| rs2295664 | SEQ ID NO: 191 | SEQ ID NO: 2557 | SEQ ID NO: 4923 |
| rs2523504 | SEQ ID NO: 192 | SEQ ID NO: 2558 | SEQ ID NO: 4924 |
| rs7381625 | SEQ ID NO: 193 | SEQ ID NO: 2559 | SEQ ID NO: 4925 |
| rs407161 | SEQ ID NO: 194 | SEQ ID NO: 2560 | SEQ ID NO: 4926 |
| rs3900268 | SEQ ID NO: 195 | SEQ ID NO: 2561 | SEQ ID NO: 4927 |
| rs3117123 | SEQ ID NO: 196 | SEQ ID NO: 2562 | SEQ ID NO: 4928 |
| rs211455 | SEQ ID NO: 197 | SEQ ID NO: 2563 | SEQ ID NO: 4929 |
| rs7451341 | SEQ ID NO: 198 | SEQ ID NO: 2564 | SEQ ID NO: 4930 |
| rs3095312 | SEQ ID NO: 199 | SEQ ID NO: 2565 | SEQ ID NO: 4931 |
| rs6932266 | SEQ ID NO: 200 | SEQ ID NO: 2566 | SEQ ID NO: 4932 |
| rs2295120 | SEQ ID NO: 201 | SEQ ID NO: 2567 | SEQ ID NO: 4933 |
| rs458679 | SEQ ID NO: 202 | SEQ ID NO: 2568 | SEQ ID NO: 4934 |
| rs763028 | SEQ ID NO: 203 | SEQ ID NO: 2569 | SEQ ID NO: 4935 |
| rs1360 | SEQ ID NO: 204 | SEQ ID NO: 2570 | SEQ ID NO: 4936 |
| rs2734574 | SEQ ID NO: 205 | SEQ ID NO: 2571 | SEQ ID NO: 4937 |
| rs1548515 | SEQ ID NO: 206 | SEQ ID NO: 2572 | SEQ ID NO: 4938 |
| rs1610736 | SEQ ID NO: 207 | SEQ ID NO: 2573 | SEQ ID NO: 4939 |
| rs1573648 | SEQ ID NO: 208 | SEQ ID NO: 2574 | SEQ ID NO: 4940 |
| rs3807035 | SEQ ID NO: 209 | SEQ ID NO: 2575 | SEQ ID NO: 4941 |
| rs3763354 | SEQ ID NO: 210 | SEQ ID NO: 2576 | SEQ ID NO: 4942 |
| rs2855437 | SEQ ID NO: 211 | SEQ ID NO: 2577 | SEQ ID NO: 4943 |
| rs3117008 | SEQ ID NO: 212 | SEQ ID NO: 2578 | SEQ ID NO: 4944 |
| rs3823342 | SEQ ID NO: 213 | SEQ ID NO: 2579 | SEQ ID NO: 4945 |
| rs2254618 | SEQ ID NO: 214 | SEQ ID NO: 2580 | SEQ ID NO: 4946 |
| rs1541270 | SEQ ID NO: 215 | SEQ ID NO: 2581 | SEQ ID NO: 4947 |
| rs3817962 | SEQ ID NO: 216 | SEQ ID NO: 2582 | SEQ ID NO: 4948 |
| rs3763313 | SEQ ID NO: 217 | SEQ ID NO: 2583 | SEQ ID NO: 4949 |
| rs1611651 | SEQ ID NO: 218 | SEQ ID NO: 2584 | SEQ ID NO: 4950 |
| rs2516713 | SEQ ID NO: 219 | SEQ ID NO: 2585 | SEQ ID NO: 4951 |
| rs3869070 | SEQ ID NO: 220 | SEQ ID NO: 2586 | SEQ ID NO: 4952 |
| rs7743412 | SEQ ID NO: 221 | SEQ ID NO: 2587 | SEQ ID NO: 4953 |
| rs1541267 | SEQ ID NO: 222 | SEQ ID NO: 2588 | SEQ ID NO: 4954 |
| rs2853977 | SEQ ID NO: 223 | SEQ ID NO: 2589 | SEQ ID NO: 4955 |
| rs3891156 | SEQ ID NO: 224 | SEQ ID NO: 2590 | SEQ ID NO: 4956 |
| rs2394607 | SEQ ID NO: 225 | SEQ ID NO: 2591 | SEQ ID NO: 4957 |
| rs1028411 | SEQ ID NO: 226 | SEQ ID NO: 2592 | SEQ ID NO: 4958 |
| rs2022065 | SEQ ID NO: 227 | SEQ ID NO: 2593 | SEQ ID NO: 4959 |
| rs2294748 | SEQ ID NO: 228 | SEQ ID NO: 2594 | SEQ ID NO: 4960 |
| rs6933541 | SEQ ID NO: 229 | SEQ ID NO: 2595 | SEQ ID NO: 4961 |
| rs1419638 | SEQ ID NO: 230 | SEQ ID NO: 2596 | SEQ ID NO: 4962 |
| rs13395 | SEQ ID NO: 231 | SEQ ID NO: 2597 | SEQ ID NO: 4963 |
| rs64036 | SEQ ID NO: 232 | SEQ ID NO: 2598 | SEQ ID NO: 4964 |
| rs727939 | SEQ ID NO: 233 | SEQ ID NO: 2599 | SEQ ID NO: 4965 |
| rs10751 | SEQ ID NO: 234 | SEQ ID NO: 2600 | SEQ ID NO: 4966 |
| rs3869065 | SEQ ID NO: 235 | SEQ ID NO: 2601 | SEQ ID NO: 4967 |
| rs3094061 | SEQ ID NO: 236 | SEQ ID NO: 2602 | SEQ ID NO: 4968 |
| rs419434 | SEQ ID NO: 237 | SEQ ID NO: 2603 | SEQ ID NO: 4969 |
| rs429916 | SEQ ID NO: 238 | SEQ ID NO: 2604 | SEQ ID NO: 4970 |
| rs3130603 | SEQ ID NO: 239 | SEQ ID NO: 2605 | SEQ ID NO: 4971 |
| rs397144 | SEQ ID NO: 240 | SEQ ID NO: 2606 | SEQ ID NO: 4972 |
| rs3873332 | SEQ ID NO: 241 | SEQ ID NO: 2607 | SEQ ID NO: 4973 |
| rs2735052 | SEQ ID NO: 242 | SEQ ID NO: 2608 | SEQ ID NO: 4974 |
| rs3134759 | SEQ ID NO: 243 | SEQ ID NO: 2609 | SEQ ID NO: 4975 |
| rs2571430 | SEQ ID NO: 244 | SEQ ID NO: 2610 | SEQ ID NO: 4976 |
| rs4713367 | SEQ ID NO: 245 | SEQ ID NO: 2611 | SEQ ID NO: 4977 |
| rs2523774 | SEQ ID NO: 246 | SEQ ID NO: 2612 | SEQ ID NO: 4978 |
| rs2233969 | SEQ ID NO: 247 | SEQ ID NO: 2613 | SEQ ID NO: 4979 |
| rs522162 | SEQ ID NO: 248 | SEQ ID NO: 2614 | SEQ ID NO: 4980 |
| rs3763324 | SEQ ID NO: 249 | SEQ ID NO: 2615 | SEQ ID NO: 4981 |
| rs1033568 | SEQ ID NO: 250 | SEQ ID NO: 2616 | SEQ ID NO: 4982 |
| rs1632984 | SEQ ID NO: 251 | SEQ ID NO: 2617 | SEQ ID NO: 4983 |
| rs1362060 | SEQ ID NO: 252 | SEQ ID NO: 2618 | SEQ ID NO: 4984 |
| rs7754443 | SEQ ID NO: 253 | SEQ ID NO: 2619 | SEQ ID NO: 4985 |
| rs2395351 | SEQ ID NO: 254 | SEQ ID NO: 2620 | SEQ ID NO: 4986 |
| rs2267644 | SEQ ID NO: 255 | SEQ ID NO: 2621 | SEQ ID NO: 4987 |
| rs29232 | SEQ ID NO: 256 | SEQ ID NO: 2622 | SEQ ID NO: 4988 |
| rs1633085 | SEQ ID NO: 257 | SEQ ID NO: 2623 | SEQ ID NO: 4989 |
| rs7743506 | SEQ ID NO: 258 | SEQ ID NO: 2624 | SEQ ID NO: 4990 |
| rs1737009 | SEQ ID NO: 259 | SEQ ID NO: 2625 | SEQ ID NO: 4991 |
| rs2844606 | SEQ ID NO: 260 | SEQ ID NO: 2626 | SEQ ID NO: 4992 |
| rs2394173 | SEQ ID NO: 261 | SEQ ID NO: 2627 | SEQ ID NO: 4993 |
| rs4392759 | SEQ ID NO: 262 | SEQ ID NO: 2628 | SEQ ID NO: 4994 |
| rs3132528 | SEQ ID NO: 263 | SEQ ID NO: 2629 | SEQ ID NO: 4995 |
| rs3130782 | SEQ ID NO: 264 | SEQ ID NO: 2630 | SEQ ID NO: 4996 |
| rs379221 | SEQ ID NO: 265 | SEQ ID NO: 2631 | SEQ ID NO: 4997 |
| rs7195 | SEQ ID NO: 266 | SEQ ID NO: 2632 | SEQ ID NO: 4998 |
| rs7383433 | SEQ ID NO: 267 | SEQ ID NO: 2633 | SEQ ID NO: 4999 |
| rs1632854 | SEQ ID NO: 268 | SEQ ID NO: 2634 | SEQ ID NO: 5000 |
| rs5007259 | SEQ ID NO: 269 | SEQ ID NO: 2635 | SEQ ID NO: 5001 |
| rs3130377 | SEQ ID NO: 270 | SEQ ID NO: 2636 | SEQ ID NO: 5002 |
| rs1419632 | SEQ ID NO: 271 | SEQ ID NO: 2637 | SEQ ID NO: 5003 |
| rs3819721 | SEQ ID NO: 272 | SEQ ID NO: 2638 | SEQ ID NO: 5004 |
| rs2071363 | SEQ ID NO: 273 | SEQ ID NO: 2639 | SEQ ID NO: 5005 |
| rs2233966 | SEQ ID NO: 274 | SEQ ID NO: 2640 | SEQ ID NO: 5006 |
| rs2621416 | SEQ ID NO: 275 | SEQ ID NO: 2641 | SEQ ID NO: 5007 |
| rs1736912 | SEQ ID NO: 276 | SEQ ID NO: 2642 | SEQ ID NO: 5008 |
| rs3763347 | SEQ ID NO: 277 | SEQ ID NO: 2643 | SEQ ID NO: 5009 |
| rs1610613 | SEQ ID NO: 278 | SEQ ID NO: 2644 | SEQ ID NO: 5010 |
| rs3135373 | SEQ ID NO: 279 | SEQ ID NO: 2645 | SEQ ID NO: 5011 |
| rs2523946 | SEQ ID NO: 280 | SEQ ID NO: 2646 | SEQ ID NO: 5012 |
| rs3873374 | SEQ ID NO: 281 | SEQ ID NO: 2647 | SEQ ID NO: 5013 |
| rs17034 | SEQ ID NO: 282 | SEQ ID NO: 2648 | SEQ ID NO: 5014 |
| rs2073149 | SEQ ID NO: 283 | SEQ ID NO: 2649 | SEQ ID NO: 5015 |
| rs2073150 | SEQ ID NO: 284 | SEQ ID NO: 2650 | SEQ ID NO: 5016 |
| rs3117007 | SEQ ID NO: 285 | SEQ ID NO: 2651 | SEQ ID NO: 5017 |
| rs3094207 | SEQ ID NO: 286 | SEQ ID NO: 2652 | SEQ ID NO: 5018 |
| rs3748082 | SEQ ID NO: 287 | SEQ ID NO: 2653 | SEQ ID NO: 5019 |
| rs423808 | SEQ ID NO: 288 | SEQ ID NO: 2654 | SEQ ID NO: 5020 |
| rs445150 | SEQ ID NO: 289 | SEQ ID NO: 2655 | SEQ ID NO: 5021 |
| rs3734841 | SEQ ID NO: 290 | SEQ ID NO: 2656 | SEQ ID NO: 5022 |
| rs3132523 | SEQ ID NO: 291 | SEQ ID NO: 2657 | SEQ ID NO: 5023 |
| rs3997854 | SEQ ID NO: 292 | SEQ ID NO: 2658 | SEQ ID NO: 5024 |
| rs2071482 | SEQ ID NO: 293 | SEQ ID NO: 2659 | SEQ ID NO: 5025 |
| rs2071469 | SEQ ID NO: 294 | SEQ ID NO: 2660 | SEQ ID NO: 5026 |
| rs2736155 | SEQ ID NO: 295 | SEQ ID NO: 2661 | SEQ ID NO: 5027 |
| rs2524198 | SEQ ID NO: 296 | SEQ ID NO: 2662 | SEQ ID NO: 5028 |
| rs560505 | SEQ ID NO: 297 | SEQ ID NO: 2663 | SEQ ID NO: 5029 |
| rs2894252 | SEQ ID NO: 298 | SEQ ID NO: 2664 | SEQ ID NO: 5030 |
| rs3094220 | SEQ ID NO: 299 | SEQ ID NO: 2665 | SEQ ID NO: 5031 |
| rs3763355 | SEQ ID NO: 300 | SEQ ID NO: 2666 | SEQ ID NO: 5032 |
| rs3823312 | SEQ ID NO: 301 | SEQ ID NO: 2667 | SEQ ID NO: 5033 |
| rs4999342 | SEQ ID NO: 302 | SEQ ID NO: 2668 | SEQ ID NO: 5034 |
| rs2517403 | SEQ ID NO: 303 | SEQ ID NO: 2669 | SEQ ID NO: 5035 |
| rs4987086 | SEQ ID NO: 304 | SEQ ID NO: 2670 | SEQ ID NO: 5036 |
| rs3819294 | SEQ ID NO: 305 | SEQ ID NO: 2671 | SEQ ID NO: 5037 |
| rs909728 | SEQ ID NO: 306 | SEQ ID NO: 2672 | SEQ ID NO: 5038 |
| rs2294477 | SEQ ID NO: 307 | SEQ ID NO: 2673 | SEQ ID NO: 5039 |
| rs3117229 | SEQ ID NO: 308 | SEQ ID NO: 2674 | SEQ ID NO: 5040 |
| rs3909178 | SEQ ID NO: 309 | SEQ ID NO: 2675 | SEQ ID NO: 5041 |
| rs3116985 | SEQ ID NO: 310 | SEQ ID NO: 2676 | SEQ ID NO: 5042 |
| rs3095320 | SEQ ID NO: 311 | SEQ ID NO: 2677 | SEQ ID NO: 5043 |
| rs241445 | SEQ ID NO: 312 | SEQ ID NO: 2678 | SEQ ID NO: 5044 |
| rs3828896 | SEQ ID NO: 313 | SEQ ID NO: 2679 | SEQ ID NO: 5045 |
| rs6914995 | SEQ ID NO: 314 | SEQ ID NO: 2680 | SEQ ID NO: 5046 |
| rs3815086 | SEQ ID NO: 315 | SEQ ID NO: 2681 | SEQ ID NO: 5047 |
| rs3132708 | SEQ ID NO: 316 | SEQ ID NO: 2682 | SEQ ID NO: 5048 |
| rs6457644 | SEQ ID NO: 317 | SEQ ID NO: 2683 | SEQ ID NO: 5049 |
| rs6914849 | SEQ ID NO: 318 | SEQ ID NO: 2684 | SEQ ID NO: 5050 |
| rs4151655 | SEQ ID NO: 319 | SEQ ID NO: 2685 | SEQ ID NO: 5051 |
| rs6457327 | SEQ ID NO: 320 | SEQ ID NO: 2686 | SEQ ID NO: 5052 |
| rs2647087 | SEQ ID NO: 321 | SEQ ID NO: 2687 | SEQ ID NO: 5053 |
| rs3830060 | SEQ ID NO: 322 | SEQ ID NO: 2688 | SEQ ID NO: 5054 |
| rs915653 | SEQ ID NO: 323 | SEQ ID NO: 2689 | SEQ ID NO: 5055 |
| rs2524059 | SEQ ID NO: 324 | SEQ ID NO: 2690 | SEQ ID NO: 5056 |
| rs6912673 | SEQ ID NO: 325 | SEQ ID NO: 2691 | SEQ ID NO: 5057 |
| rs4713218 | SEQ ID NO: 326 | SEQ ID NO: 2692 | SEQ ID NO: 5058 |
| rs3135372 | SEQ ID NO: 327 | SEQ ID NO: 2693 | SEQ ID NO: 5059 |
| rs3807034 | SEQ ID NO: 328 | SEQ ID NO: 2694 | SEQ ID NO: 5060 |
| rs2158285 | SEQ ID NO: 329 | SEQ ID NO: 2695 | SEQ ID NO: 5061 |
| rs2735061 | SEQ ID NO: 330 | SEQ ID NO: 2696 | SEQ ID NO: 5062 |
| rs204995 | SEQ ID NO: 331 | SEQ ID NO: 2697 | SEQ ID NO: 5063 |
| rs2596501 | SEQ ID NO: 332 | SEQ ID NO: 2698 | SEQ ID NO: 5064 |
| rs2256902 | SEQ ID NO: 333 | SEQ ID NO: 2699 | SEQ ID NO: 5065 |
| rs2844586 | SEQ ID NO: 334 | SEQ ID NO: 2700 | SEQ ID NO: 5066 |
| rs2293861 | SEQ ID NO: 335 | SEQ ID NO: 2701 | SEQ ID NO: 5067 |
| rs1233408 | SEQ ID NO: 336 | SEQ ID NO: 2702 | SEQ ID NO: 5068 |
| rs7382794 | SEQ ID NO: 337 | SEQ ID NO: 2703 | SEQ ID NO: 5069 |
| rs3830061 | SEQ ID NO: 338 | SEQ ID NO: 2704 | SEQ ID NO: 5070 |
| rs2524074 | SEQ ID NO: 339 | SEQ ID NO: 2705 | SEQ ID NO: 5071 |
| rs3131025 | SEQ ID NO: 340 | SEQ ID NO: 2706 | SEQ ID NO: 5072 |
| rs1034324 | SEQ ID NO: 341 | SEQ ID NO: 2707 | SEQ ID NO: 5073 |
| rs3130655 | SEQ ID NO: 342 | SEQ ID NO: 2708 | SEQ ID NO: 5074 |
| rs4713324 | SEQ ID NO: 343 | SEQ ID NO: 2709 | SEQ ID NO: 5075 |
| rs3129888 | SEQ ID NO: 344 | SEQ ID NO: 2710 | SEQ ID NO: 5076 |
| rs2071366 | SEQ ID NO: 345 | SEQ ID NO: 2711 | SEQ ID NO: 5077 |
| rs2074479 | SEQ ID NO: 346 | SEQ ID NO: 2712 | SEQ ID NO: 5078 |
| rs2516312 | SEQ ID NO: 347 | SEQ ID NO: 2713 | SEQ ID NO: 5079 |
| rs1737078 | SEQ ID NO: 348 | SEQ ID NO: 2714 | SEQ ID NO: 5080 |
| rs3734854 | SEQ ID NO: 349 | SEQ ID NO: 2715 | SEQ ID NO: 5081 |
| rs6901541 | SEQ ID NO: 350 | SEQ ID NO: 2716 | SEQ ID NO: 5082 |
| rs3916766 | SEQ ID NO: 351 | SEQ ID NO: 2717 | SEQ ID NO: 5083 |
| rs6919972 | SEQ ID NO: 352 | SEQ ID NO: 2718 | SEQ ID NO: 5084 |
| rs6911639 | SEQ ID NO: 353 | SEQ ID NO: 2719 | SEQ ID NO: 5085 |
| rs6921314 | SEQ ID NO: 354 | SEQ ID NO: 2720 | SEQ ID NO: 5086 |
| rs3135351 | SEQ ID NO: 355 | SEQ ID NO: 2721 | SEQ ID NO: 5087 |
| rs3129299 | SEQ ID NO: 356 | SEQ ID NO: 2722 | SEQ ID NO: 5088 |
| rs7745235 | SEQ ID NO: 357 | SEQ ID NO: 2723 | SEQ ID NO: 5089 |
| rs3819285 | SEQ ID NO: 358 | SEQ ID NO: 2724 | SEQ ID NO: 5090 |
| rs1233493 | SEQ ID NO: 359 | SEQ ID NO: 2725 | SEQ ID NO: 5091 |
| rs2066744 | SEQ ID NO: 360 | SEQ ID NO: 2726 | SEQ ID NO: 5092 |
| rs1367728 | SEQ ID NO: 361 | SEQ ID NO: 2727 | SEQ ID NO: 5093 |
| rs2853960 | SEQ ID NO: 362 | SEQ ID NO: 2728 | SEQ ID NO: 5094 |
| rs4151672 | SEQ ID NO: 363 | SEQ ID NO: 2729 | SEQ ID NO: 5095 |
| rs3891157 | SEQ ID NO: 364 | SEQ ID NO: 2730 | SEQ ID NO: 5096 |
| rs2394255 | SEQ ID NO: 365 | SEQ ID NO: 2731 | SEQ ID NO: 5097 |
| rs3128967 | SEQ ID NO: 366 | SEQ ID NO: 2732 | SEQ ID NO: 5098 |
| rs3130106 | SEQ ID NO: 367 | SEQ ID NO: 2733 | SEQ ID NO: 5099 |
| rs1059231 | SEQ ID NO: 368 | SEQ ID NO: 2734 | SEQ ID NO: 5100 |
| rs356966 | SEQ ID NO: 369 | SEQ ID NO: 2735 | SEQ ID NO: 5101 |
| rs2394605 | SEQ ID NO: 370 | SEQ ID NO: 2736 | SEQ ID NO: 5102 |
| rs2295118 | SEQ ID NO: 371 | SEQ ID NO: 2737 | SEQ ID NO: 5103 |
| rs2050191 | SEQ ID NO: 372 | SEQ ID NO: 2738 | SEQ ID NO: 5104 |
| rs427417 | SEQ ID NO: 373 | SEQ ID NO: 2739 | SEQ ID NO: 5105 |
| rs3828808 | SEQ ID NO: 374 | SEQ ID NO: 2740 | SEQ ID NO: 5106 |
| rs7383005 | SEQ ID NO: 375 | SEQ ID NO: 2741 | SEQ ID NO: 5107 |
| rs6940585 | SEQ ID NO: 376 | SEQ ID NO: 2742 | SEQ ID NO: 5108 |
| rs2294745 | SEQ ID NO: 377 | SEQ ID NO: 2743 | SEQ ID NO: 5109 |
| rs3128854 | SEQ ID NO: 378 | SEQ ID NO: 2744 | SEQ ID NO: 5110 |
| rs1633037 | SEQ ID NO: 379 | SEQ ID NO: 2745 | SEQ ID NO: 5111 |
| rs3132550 | SEQ ID NO: 380 | SEQ ID NO: 2746 | SEQ ID NO: 5112 |
| rs1610708 | SEQ ID NO: 381 | SEQ ID NO: 2747 | SEQ ID NO: 5113 |
| rs2844766 | SEQ ID NO: 382 | SEQ ID NO: 2748 | SEQ ID NO: 5114 |
| rs4713447 | SEQ ID NO: 383 | SEQ ID NO: 2749 | SEQ ID NO: 5115 |
| rs4151671 | SEQ ID NO: 384 | SEQ ID NO: 2750 | SEQ ID NO: 5116 |
| rs733840 | SEQ ID NO: 385 | SEQ ID NO: 2751 | SEQ ID NO: 5117 |
| rs3198005 | SEQ ID NO: 386 | SEQ ID NO: 2752 | SEQ ID NO: 5118 |
| rs2523814 | SEQ ID NO: 387 | SEQ ID NO: 2753 | SEQ ID NO: 5119 |
| rs7755580 | SEQ ID NO: 388 | SEQ ID NO: 2754 | SEQ ID NO: 5120 |
| rs2071364 | SEQ ID NO: 389 | SEQ ID NO: 2755 | SEQ ID NO: 5121 |
| rs2894239 | SEQ ID NO: 390 | SEQ ID NO: 2756 | SEQ ID NO: 5122 |
| rs3117230 | SEQ ID NO: 391 | SEQ ID NO: 2757 | SEQ ID NO: 5123 |
| rs1628578 | SEQ ID NO: 392 | SEQ ID NO: 2758 | SEQ ID NO: 5124 |
| rs362531 | SEQ ID NO: 393 | SEQ ID NO: 2759 | SEQ ID NO: 5125 |
| rs6899851 | SEQ ID NO: 394 | SEQ ID NO: 2760 | SEQ ID NO: 5126 |
| rs4084091 | SEQ ID NO: 395 | SEQ ID NO: 2761 | SEQ ID NO: 5127 |
| rs2735097 | SEQ ID NO: 396 | SEQ ID NO: 2762 | SEQ ID NO: 5128 |
| rs1264698 | SEQ ID NO: 397 | SEQ ID NO: 2763 | SEQ ID NO: 5129 |
| rs2071789 | SEQ ID NO: 398 | SEQ ID NO: 2764 | SEQ ID NO: 5130 |
| rs3093544 | SEQ ID NO: 399 | SEQ ID NO: 2765 | SEQ ID NO: 5131 |
| rs3132130 | SEQ ID NO: 400 | SEQ ID NO: 2766 | SEQ ID NO: 5132 |
| rs3093669 | SEQ ID NO: 401 | SEQ ID NO: 2767 | SEQ ID NO: 5133 |
| rs2092305 | SEQ ID NO: 402 | SEQ ID NO: 2768 | SEQ ID NO: 5134 |
| rs3128927 | SEQ ID NO: 403 | SEQ ID NO: 2769 | SEQ ID NO: 5135 |
| rs1633009 | SEQ ID NO: 404 | SEQ ID NO: 2770 | SEQ ID NO: 5136 |
| rs6914878 | SEQ ID NO: 405 | SEQ ID NO: 2771 | SEQ ID NO: 5137 |
| rs8337 | SEQ ID NO: 406 | SEQ ID NO: 2772 | SEQ ID NO: 5138 |
| rs2286712 | SEQ ID NO: 407 | SEQ ID NO: 2773 | SEQ ID NO: 5139 |
| rs1633086 | SEQ ID NO: 408 | SEQ ID NO: 2774 | SEQ ID NO: 5140 |
| rs2245514 | SEQ ID NO: 409 | SEQ ID NO: 2775 | SEQ ID NO: 5141 |
| rs1265086 | SEQ ID NO: 410 | SEQ ID NO: 2776 | SEQ ID NO: 5142 |
| rs466384 | SEQ ID NO: 411 | SEQ ID NO: 2777 | SEQ ID NO: 5143 |
| rs3117225 | SEQ ID NO: 412 | SEQ ID NO: 2778 | SEQ ID NO: 5144 |
| rs209473 | SEQ ID NO: 413 | SEQ ID NO: 2779 | SEQ ID NO: 5145 |
| rs4151664 | SEQ ID NO: 414 | SEQ ID NO: 2780 | SEQ ID NO: 5146 |
| rs909967 | SEQ ID NO: 415 | SEQ ID NO: 2781 | SEQ ID NO: 5147 |
| rs1633013 | SEQ ID NO: 416 | SEQ ID NO: 2782 | SEQ ID NO: 5148 |
| rs4151657 | SEQ ID NO: 417 | SEQ ID NO: 2783 | SEQ ID NO: 5149 |
| rs1419689 | SEQ ID NO: 418 | SEQ ID NO: 2784 | SEQ ID NO: 5150 |
| rs3757333 | SEQ ID NO: 419 | SEQ ID NO: 2785 | SEQ ID NO: 5151 |
| rs1264541 | SEQ ID NO: 420 | SEQ ID NO: 2786 | SEQ ID NO: 5152 |
| rs7992 | SEQ ID NO: 421 | SEQ ID NO: 2787 | SEQ ID NO: 5153 |
| rs2523926 | SEQ ID NO: 422 | SEQ ID NO: 2788 | SEQ ID NO: 5154 |
| rs3823419 | SEQ ID NO: 423 | SEQ ID NO: 2789 | SEQ ID NO: 5155 |
| rs2735003 | SEQ ID NO: 424 | SEQ ID NO: 2790 | SEQ ID NO: 5156 |
| rs3817965 | SEQ ID NO: 425 | SEQ ID NO: 2791 | SEQ ID NO: 5157 |
| rs2076540 | SEQ ID NO: 426 | SEQ ID NO: 2792 | SEQ ID NO: 5158 |
| rs3998799 | SEQ ID NO: 427 | SEQ ID NO: 2793 | SEQ ID NO: 5159 |
| rs2428510 | SEQ ID NO: 428 | SEQ ID NO: 2794 | SEQ ID NO: 5160 |
| rs3094187 | SEQ ID NO: 429 | SEQ ID NO: 2795 | SEQ ID NO: 5161 |
| rs2076484 | SEQ ID NO: 430 | SEQ ID NO: 2796 | SEQ ID NO: 5162 |
| rs3763349 | SEQ ID NO: 431 | SEQ ID NO: 2797 | SEQ ID NO: 5163 |
| rs2523512 | SEQ ID NO: 432 | SEQ ID NO: 2798 | SEQ ID NO: 5164 |
| rs2517439 | SEQ ID NO: 433 | SEQ ID NO: 2799 | SEQ ID NO: 5165 |
| rs1419634 | SEQ ID NO: 434 | SEQ ID NO: 2800 | SEQ ID NO: 5166 |
| rs241438 | SEQ ID NO: 435 | SEQ ID NO: 2801 | SEQ ID NO: 5167 |
| rs2244839 | SEQ ID NO: 436 | SEQ ID NO: 2802 | SEQ ID NO: 5168 |
| rs3135033 | SEQ ID NO: 437 | SEQ ID NO: 2803 | SEQ ID NO: 5169 |
| rs3135388 | SEQ ID NO: 438 | SEQ ID NO: 2804 | SEQ ID NO: 5170 |
| rs3807033 | SEQ ID NO: 439 | SEQ ID NO: 2805 | SEQ ID NO: 5171 |
| rs4713446 | SEQ ID NO: 440 | SEQ ID NO: 2806 | SEQ ID NO: 5172 |
| rs3115608 | SEQ ID NO: 441 | SEQ ID NO: 2807 | SEQ ID NO: 5173 |
| rs1736994 | SEQ ID NO: 442 | SEQ ID NO: 2808 | SEQ ID NO: 5174 |
| rs6913309 | SEQ ID NO: 443 | SEQ ID NO: 2809 | SEQ ID NO: 5175 |
| rs6905408 | SEQ ID NO: 444 | SEQ ID NO: 2810 | SEQ ID NO: 5176 |
| rs3128917 | SEQ ID NO: 445 | SEQ ID NO: 2811 | SEQ ID NO: 5177 |
| rs6934041 | SEQ ID NO: 446 | SEQ ID NO: 2812 | SEQ ID NO: 5178 |
| rs6916248 | SEQ ID NO: 447 | SEQ ID NO: 2813 | SEQ ID NO: 5179 |
| rs5007263 | SEQ ID NO: 448 | SEQ ID NO: 2814 | SEQ ID NO: 5180 |
| rs3819300 | SEQ ID NO: 449 | SEQ ID NO: 2815 | SEQ ID NO: 5181 |
| rs2071471 | SEQ ID NO: 450 | SEQ ID NO: 2816 | SEQ ID NO: 5182 |
| rs2894179 | SEQ ID NO: 451 | SEQ ID NO: 2817 | SEQ ID NO: 5183 |
| rs2239705 | SEQ ID NO: 452 | SEQ ID NO: 2818 | SEQ ID NO: 5184 |
| rs2301220 | SEQ ID NO: 453 | SEQ ID NO: 2819 | SEQ ID NO: 5185 |
| rs2272904 | SEQ ID NO: 454 | SEQ ID NO: 2820 | SEQ ID NO: 5186 |
| rs6935418 | SEQ ID NO: 455 | SEQ ID NO: 2821 | SEQ ID NO: 5187 |
| rs5021450 | SEQ ID NO: 456 | SEQ ID NO: 2822 | SEQ ID NO: 5188 |
| rs2857103 | SEQ ID NO: 457 | SEQ ID NO: 2823 | SEQ ID NO: 5189 |
| rs2853953 | SEQ ID NO: 458 | SEQ ID NO: 2824 | SEQ ID NO: 5190 |
| rs757259 | SEQ ID NO: 459 | SEQ ID NO: 2825 | SEQ ID NO: 5191 |
| rs3135391 | SEQ ID NO: 460 | SEQ ID NO: 2826 | SEQ ID NO: 5192 |
| rs2523606 | SEQ ID NO: 461 | SEQ ID NO: 2827 | SEQ ID NO: 5193 |
| rs1632962 | SEQ ID NO: 462 | SEQ ID NO: 2828 | SEQ ID NO: 5194 |
| rs3135353 | SEQ ID NO: 463 | SEQ ID NO: 2829 | SEQ ID NO: 5195 |
| rs3130235 | SEQ ID NO: 464 | SEQ ID NO: 2830 | SEQ ID NO: 5196 |
| rs2394606 | SEQ ID NO: 465 | SEQ ID NO: 2831 | SEQ ID NO: 5197 |
| rs2734997 | SEQ ID NO: 466 | SEQ ID NO: 2832 | SEQ ID NO: 5198 |
| rs3998794 | SEQ ID NO: 467 | SEQ ID NO: 2833 | SEQ ID NO: 5199 |
| rs2844463 | SEQ ID NO: 468 | SEQ ID NO: 2834 | SEQ ID NO: 5200 |
| rs7452233 | SEQ ID NO: 469 | SEQ ID NO: 2835 | SEQ ID NO: 5201 |
| rs6910598 | SEQ ID NO: 470 | SEQ ID NO: 2836 | SEQ ID NO: 5202 |
| rs3129008 | SEQ ID NO: 471 | SEQ ID NO: 2837 | SEQ ID NO: 5203 |
| rs1150741 | SEQ ID NO: 472 | SEQ ID NO: 2838 | SEQ ID NO: 5204 |
| rs7774452 | SEQ ID NO: 473 | SEQ ID NO: 2839 | SEQ ID NO: 5205 |
| rs6911724 | SEQ ID NO: 474 | SEQ ID NO: 2840 | SEQ ID NO: 5206 |
| rs2294479 | SEQ ID NO: 475 | SEQ ID NO: 2841 | SEQ ID NO: 5207 |
| rs417374 | SEQ ID NO: 476 | SEQ ID NO: 2842 | SEQ ID NO: 5208 |
| rs1633030 | SEQ ID NO: 477 | SEQ ID NO: 2843 | SEQ ID NO: 5209 |
| rs2024679 | SEQ ID NO: 478 | SEQ ID NO: 2844 | SEQ ID NO: 5210 |
| rs3116999 | SEQ ID NO: 479 | SEQ ID NO: 2845 | SEQ ID NO: 5211 |
| rs3765605 | SEQ ID NO: 480 | SEQ ID NO: 2846 | SEQ ID NO: 5212 |
| rs2844603 | SEQ ID NO: 481 | SEQ ID NO: 2847 | SEQ ID NO: 5213 |
| rs2621419 | SEQ ID NO: 482 | SEQ ID NO: 2848 | SEQ ID NO: 5214 |
| rs1362073 | SEQ ID NO: 483 | SEQ ID NO: 2849 | SEQ ID NO: 5215 |
| rs2072634 | SEQ ID NO: 484 | SEQ ID NO: 2850 | SEQ ID NO: 5216 |
| rs213208 | SEQ ID NO: 485 | SEQ ID NO: 2851 | SEQ ID NO: 5217 |
| rs3956934 | SEQ ID NO: 486 | SEQ ID NO: 2852 | SEQ ID NO: 5218 |
| rs3117227 | SEQ ID NO: 487 | SEQ ID NO: 2853 | SEQ ID NO: 5219 |
| rs4713215 | SEQ ID NO: 488 | SEQ ID NO: 2854 | SEQ ID NO: 5220 |
| rs1632942 | SEQ ID NO: 489 | SEQ ID NO: 2855 | SEQ ID NO: 5221 |
| rs3129886 | SEQ ID NO: 490 | SEQ ID NO: 2856 | SEQ ID NO: 5222 |
| rs2071798 | SEQ ID NO: 491 | SEQ ID NO: 2857 | SEQ ID NO: 5223 |
| rs2523586 | SEQ ID NO: 492 | SEQ ID NO: 2858 | SEQ ID NO: 5224 |
| rs3117234 | SEQ ID NO: 493 | SEQ ID NO: 2859 | SEQ ID NO: 5225 |
| rs2071473 | SEQ ID NO: 494 | SEQ ID NO: 2860 | SEQ ID NO: 5226 |
| rs3094041 | SEQ ID NO: 495 | SEQ ID NO: 2861 | SEQ ID NO: 5227 |
| rs2596472 | SEQ ID NO: 496 | SEQ ID NO: 2862 | SEQ ID NO: 5228 |
| rs2213570 | SEQ ID NO: 497 | SEQ ID NO: 2863 | SEQ ID NO: 5229 |
| rs1736922 | SEQ ID NO: 498 | SEQ ID NO: 2864 | SEQ ID NO: 5230 |
| rs3129269 | SEQ ID NO: 499 | SEQ ID NO: 2865 | SEQ ID NO: 5231 |
| rs6920457 | SEQ ID NO: 500 | SEQ ID NO: 2866 | SEQ ID NO: 5232 |
| rs4711229 | SEQ ID NO: 501 | SEQ ID NO: 2867 | SEQ ID NO: 5233 |
| rs1799964 | SEQ ID NO: 502 | SEQ ID NO: 2868 | SEQ ID NO: 5234 |
| rs4959037 | SEQ ID NO: 503 | SEQ ID NO: 2869 | SEQ ID NO: 5235 |
| rs1611652 | SEQ ID NO: 504 | SEQ ID NO: 2870 | SEQ ID NO: 5236 |
| rs2071354 | SEQ ID NO: 505 | SEQ ID NO: 2871 | SEQ ID NO: 5237 |
| rs1737012 | SEQ ID NO: 506 | SEQ ID NO: 2872 | SEQ ID NO: 5238 |
| rs1988564 | SEQ ID NO: 507 | SEQ ID NO: 2873 | SEQ ID NO: 5239 |
| rs2071790 | SEQ ID NO: 508 | SEQ ID NO: 2874 | SEQ ID NO: 5240 |
| rs465151 | SEQ ID NO: 509 | SEQ ID NO: 2875 | SEQ ID NO: 5241 |
| rs1265759 | SEQ ID NO: 510 | SEQ ID NO: 2876 | SEQ ID NO: 5242 |
| rs2239701 | SEQ ID NO: 511 | SEQ ID NO: 2877 | SEQ ID NO: 5243 |
| rs3129883 | SEQ ID NO: 512 | SEQ ID NO: 2878 | SEQ ID NO: 5244 |
| rs2032502 | SEQ ID NO: 513 | SEQ ID NO: 2879 | SEQ ID NO: 5245 |
| rs3778612 | SEQ ID NO: 514 | SEQ ID NO: 2880 | SEQ ID NO: 5246 |
| rs2246871 | SEQ ID NO: 515 | SEQ ID NO: 2881 | SEQ ID NO: 5247 |
| rs2239707 | SEQ ID NO: 516 | SEQ ID NO: 2882 | SEQ ID NO: 5248 |
| rs2734916 | SEQ ID NO: 517 | SEQ ID NO: 2883 | SEQ ID NO: 5249 |
| rs2301753 | SEQ ID NO: 518 | SEQ ID NO: 2884 | SEQ ID NO: 5250 |
| rs2735042 | SEQ ID NO: 519 | SEQ ID NO: 2885 | SEQ ID NO: 5251 |
| rs3179375 | SEQ ID NO: 520 | SEQ ID NO: 2886 | SEQ ID NO: 5252 |
| rs3129869 | SEQ ID NO: 521 | SEQ ID NO: 2887 | SEQ ID NO: 5253 |
| rs7143 | SEQ ID NO: 522 | SEQ ID NO: 2888 | SEQ ID NO: 5254 |
| rs2068204 | SEQ ID NO: 523 | SEQ ID NO: 2889 | SEQ ID NO: 5255 |
| rs2745411 | SEQ ID NO: 524 | SEQ ID NO: 2890 | SEQ ID NO: 5256 |
| rs462093 | SEQ ID NO: 525 | SEQ ID NO: 2891 | SEQ ID NO: 5257 |
| rs3817966 | SEQ ID NO: 526 | SEQ ID NO: 2892 | SEQ ID NO: 5258 |
| rs2256919 | SEQ ID NO: 527 | SEQ ID NO: 2893 | SEQ ID NO: 5259 |
| rs3957010 | SEQ ID NO: 528 | SEQ ID NO: 2894 | SEQ ID NO: 5260 |
| rs1610710 | SEQ ID NO: 529 | SEQ ID NO: 2895 | SEQ ID NO: 5261 |
| rs2735067 | SEQ ID NO: 530 | SEQ ID NO: 2896 | SEQ ID NO: 5262 |
| rs3128963 | SEQ ID NO: 531 | SEQ ID NO: 2897 | SEQ ID NO: 5263 |
| rs7382938 | SEQ ID NO: 532 | SEQ ID NO: 2898 | SEQ ID NO: 5264 |
| rs3828901 | SEQ ID NO: 533 | SEQ ID NO: 2899 | SEQ ID NO: 5265 |
| rs3131043 | SEQ ID NO: 534 | SEQ ID NO: 2900 | SEQ ID NO: 5266 |
| rs2107197 | SEQ ID NO: 535 | SEQ ID NO: 2901 | SEQ ID NO: 5267 |
| rs3106190 | SEQ ID NO: 536 | SEQ ID NO: 2902 | SEQ ID NO: 5268 |
| rs1633102 | SEQ ID NO: 537 | SEQ ID NO: 2903 | SEQ ID NO: 5269 |
| rs3117120 | SEQ ID NO: 538 | SEQ ID NO: 2904 | SEQ ID NO: 5270 |
| rs4713610 | SEQ ID NO: 539 | SEQ ID NO: 2905 | SEQ ID NO: 5271 |
| rs2076522 | SEQ ID NO: 540 | SEQ ID NO: 2906 | SEQ ID NO: 5272 |
| rs2853937 | SEQ ID NO: 541 | SEQ ID NO: 2907 | SEQ ID NO: 5273 |
| rs887464 | SEQ ID NO: 542 | SEQ ID NO: 2908 | SEQ ID NO: 5274 |
| rs2074466 | SEQ ID NO: 543 | SEQ ID NO: 2909 | SEQ ID NO: 5275 |
| rs7382649 | SEQ ID NO: 544 | SEQ ID NO: 2910 | SEQ ID NO: 5276 |
| rs7452209 | SEQ ID NO: 545 | SEQ ID NO: 2911 | SEQ ID NO: 5277 |
| rs5007238 | SEQ ID NO: 546 | SEQ ID NO: 2912 | SEQ ID NO: 5278 |
| rs757262 | SEQ ID NO: 547 | SEQ ID NO: 2913 | SEQ ID NO: 5279 |
| rs1611407 | SEQ ID NO: 548 | SEQ ID NO: 2914 | SEQ ID NO: 5280 |
| rs1655937 | SEQ ID NO: 549 | SEQ ID NO: 2915 | SEQ ID NO: 5281 |
| rs2269704 | SEQ ID NO: 550 | SEQ ID NO: 2916 | SEQ ID NO: 5282 |
| rs2734331 | SEQ ID NO: 551 | SEQ ID NO: 2917 | SEQ ID NO: 5283 |
| rs3998178 | SEQ ID NO: 552 | SEQ ID NO: 2918 | SEQ ID NO: 5284 |
| rs885914 | SEQ ID NO: 553 | SEQ ID NO: 2919 | SEQ ID NO: 5285 |
| rs6925041 | SEQ ID NO: 554 | SEQ ID NO: 2920 | SEQ ID NO: 5286 |
| rs441126 | SEQ ID NO: 555 | SEQ ID NO: 2921 | SEQ ID NO: 5287 |
| rs2394604 | SEQ ID NO: 556 | SEQ ID NO: 2922 | SEQ ID NO: 5288 |
| rs2074502 | SEQ ID NO: 557 | SEQ ID NO: 2923 | SEQ ID NO: 5289 |
| rs3815100 | SEQ ID NO: 558 | SEQ ID NO: 2924 | SEQ ID NO: 5290 |
| rs1632932 | SEQ ID NO: 559 | SEQ ID NO: 2925 | SEQ ID NO: 5291 |
| rs2076524 | SEQ ID NO: 560 | SEQ ID NO: 2926 | SEQ ID NO: 5292 |
| rs7759127 | SEQ ID NO: 561 | SEQ ID NO: 2927 | SEQ ID NO: 5293 |
| rs3765606 | SEQ ID NO: 562 | SEQ ID NO: 2928 | SEQ ID NO: 5294 |
| rs7381988 | SEQ ID NO: 563 | SEQ ID NO: 2929 | SEQ ID NO: 5295 |
| rs2071479 | SEQ ID NO: 564 | SEQ ID NO: 2930 | SEQ ID NO: 5296 |
| rs4986978 | SEQ ID NO: 565 | SEQ ID NO: 2931 | SEQ ID NO: 5297 |
| rs2071472 | SEQ ID NO: 566 | SEQ ID NO: 2932 | SEQ ID NO: 5298 |
| rs7349863 | SEQ ID NO: 567 | SEQ ID NO: 2933 | SEQ ID NO: 5299 |
| rs3093665 | SEQ ID NO: 568 | SEQ ID NO: 2934 | SEQ ID NO: 5300 |
| rs3132132 | SEQ ID NO: 569 | SEQ ID NO: 2935 | SEQ ID NO: 5301 |
| rs3823318 | SEQ ID NO: 570 | SEQ ID NO: 2936 | SEQ ID NO: 5302 |
| rs2106072 | SEQ ID NO: 571 | SEQ ID NO: 2937 | SEQ ID NO: 5303 |
| rs3130654 | SEQ ID NO: 572 | SEQ ID NO: 2938 | SEQ ID NO: 5304 |
| rs2857130 | SEQ ID NO: 573 | SEQ ID NO: 2939 | SEQ ID NO: 5305 |
| rs1264300 | SEQ ID NO: 574 | SEQ ID NO: 2940 | SEQ ID NO: 5306 |
| rs909727 | SEQ ID NO: 575 | SEQ ID NO: 2941 | SEQ ID NO: 5307 |
| rs4151670 | SEQ ID NO: 576 | SEQ ID NO: 2942 | SEQ ID NO: 5308 |
| rs1419637 | SEQ ID NO: 577 | SEQ ID NO: 2943 | SEQ ID NO: 5309 |
| rs2532934 | SEQ ID NO: 578 | SEQ ID NO: 2944 | SEQ ID NO: 5310 |
| rs1632981 | SEQ ID NO: 579 | SEQ ID NO: 2945 | SEQ ID NO: 5311 |
| rs7383287 | SEQ ID NO: 580 | SEQ ID NO: 2946 | SEQ ID NO: 5312 |
| rs2071477 | SEQ ID NO: 581 | SEQ ID NO: 2947 | SEQ ID NO: 5313 |
| rs2071652 | SEQ ID NO: 582 | SEQ ID NO: 2948 | SEQ ID NO: 5314 |
| rs7756516 | SEQ ID NO: 583 | SEQ ID NO: 2949 | SEQ ID NO: 5315 |
| rs2734981 | SEQ ID NO: 584 | SEQ ID NO: 2950 | SEQ ID NO: 5316 |
| rs3823339 | SEQ ID NO: 585 | SEQ ID NO: 2951 | SEQ ID NO: 5317 |
| rs7770557 | SEQ ID NO: 586 | SEQ ID NO: 2952 | SEQ ID NO: 5318 |
| rs1054175 | SEQ ID NO: 587 | SEQ ID NO: 2953 | SEQ ID NO: 5319 |
| rs3131786 | SEQ ID NO: 588 | SEQ ID NO: 2954 | SEQ ID NO: 5320 |
| rs2844686 | SEQ ID NO: 589 | SEQ ID NO: 2955 | SEQ ID NO: 5321 |
| rs1611653 | SEQ ID NO: 590 | SEQ ID NO: 2956 | SEQ ID NO: 5322 |
| rs2242655 | SEQ ID NO: 591 | SEQ ID NO: 2957 | SEQ ID NO: 5323 |
| rs969891 | SEQ ID NO: 592 | SEQ ID NO: 2958 | SEQ ID NO: 5324 |
| rs2023609 | SEQ ID NO: 593 | SEQ ID NO: 2959 | SEQ ID NO: 5325 |
| rs3129882 | SEQ ID NO: 594 | SEQ ID NO: 2960 | SEQ ID NO: 5326 |
| rs3132553 | SEQ ID NO: 595 | SEQ ID NO: 2961 | SEQ ID NO: 5327 |
| rs2233956 | SEQ ID NO: 596 | SEQ ID NO: 2962 | SEQ ID NO: 5328 |
| rs3128930 | SEQ ID NO: 597 | SEQ ID NO: 2963 | SEQ ID NO: 5329 |
| rs3130560 | SEQ ID NO: 598 | SEQ ID NO: 2964 | SEQ ID NO: 5330 |
| rs1042141 | SEQ ID NO: 599 | SEQ ID NO: 2965 | SEQ ID NO: 5331 |
| rs2256543 | SEQ ID NO: 600 | SEQ ID NO: 2966 | SEQ ID NO: 5332 |
| rs2071362 | SEQ ID NO: 601 | SEQ ID NO: 2967 | SEQ ID NO: 5333 |
| rs4711313 | SEQ ID NO: 602 | SEQ ID NO: 2968 | SEQ ID NO: 5334 |
| rs916568 | SEQ ID NO: 603 | SEQ ID NO: 2969 | SEQ ID NO: 5335 |
| rs4988889 | SEQ ID NO: 604 | SEQ ID NO: 2970 | SEQ ID NO: 5336 |
| rs3132524 | SEQ ID NO: 605 | SEQ ID NO: 2971 | SEQ ID NO: 5337 |
| rs1033569 | SEQ ID NO: 606 | SEQ ID NO: 2972 | SEQ ID NO: 5338 |
| rs4713242 | SEQ ID NO: 607 | SEQ ID NO: 2973 | SEQ ID NO: 5339 |
| rs3095334 | SEQ ID NO: 608 | SEQ ID NO: 2974 | SEQ ID NO: 5340 |
| rs2844666 | SEQ ID NO: 609 | SEQ ID NO: 2975 | SEQ ID NO: 5341 |
| rs2517671 | SEQ ID NO: 610 | SEQ ID NO: 2976 | SEQ ID NO: 5342 |
| rs6903130 | SEQ ID NO: 611 | SEQ ID NO: 2977 | SEQ ID NO: 5343 |
| rs2179919 | SEQ ID NO: 612 | SEQ ID NO: 2978 | SEQ ID NO: 5344 |
| rs2233984 | SEQ ID NO: 613 | SEQ ID NO: 2979 | SEQ ID NO: 5345 |
| rs3117574 | SEQ ID NO: 614 | SEQ ID NO: 2980 | SEQ ID NO: 5346 |
| rs356969 | SEQ ID NO: 615 | SEQ ID NO: 2981 | SEQ ID NO: 5347 |
| rs3128972 | SEQ ID NO: 616 | SEQ ID NO: 2982 | SEQ ID NO: 5348 |
| rs4084093 | SEQ ID NO: 617 | SEQ ID NO: 2983 | SEQ ID NO: 5349 |
| rs805262 | SEQ ID NO: 618 | SEQ ID NO: 2984 | SEQ ID NO: 5350 |
| rs2269555 | SEQ ID NO: 619 | SEQ ID NO: 2985 | SEQ ID NO: 5351 |
| rs3095298 | SEQ ID NO: 620 | SEQ ID NO: 2986 | SEQ ID NO: 5352 |
| rs189865 | SEQ ID NO: 621 | SEQ ID NO: 2987 | SEQ ID NO: 5353 |
| rs3130991 | SEQ ID NO: 622 | SEQ ID NO: 2988 | SEQ ID NO: 5354 |
| rs1061235 | SEQ ID NO: 623 | SEQ ID NO: 2989 | SEQ ID NO: 5355 |
| rs3823421 | SEQ ID NO: 624 | SEQ ID NO: 2990 | SEQ ID NO: 5356 |
| rs4079877 | SEQ ID NO: 625 | SEQ ID NO: 2991 | SEQ ID NO: 5357 |
| rs909968 | SEQ ID NO: 626 | SEQ ID NO: 2992 | SEQ ID NO: 5358 |
| rs3218807 | SEQ ID NO: 627 | SEQ ID NO: 2993 | SEQ ID NO: 5359 |
| rs760293 | SEQ ID NO: 628 | SEQ ID NO: 2994 | SEQ ID NO: 5360 |
| rs5026743 | SEQ ID NO: 629 | SEQ ID NO: 2995 | SEQ ID NO: 5361 |
| rs2213586 | SEQ ID NO: 630 | SEQ ID NO: 2996 | SEQ ID NO: 5362 |
| rs3132709 | SEQ ID NO: 631 | SEQ ID NO: 2997 | SEQ ID NO: 5363 |
| rs805287 | SEQ ID NO: 632 | SEQ ID NO: 2998 | SEQ ID NO: 5364 |
| rs391755 | SEQ ID NO: 633 | SEQ ID NO: 2999 | SEQ ID NO: 5365 |
| rs3765604 | SEQ ID NO: 634 | SEQ ID NO: 3000 | SEQ ID NO: 5366 |
| rs1265087 | SEQ ID NO: 635 | SEQ ID NO: 3001 | SEQ ID NO: 5367 |
| rs516535 | SEQ ID NO: 636 | SEQ ID NO: 3002 | SEQ ID NO: 5368 |
| rs1632883 | SEQ ID NO: 637 | SEQ ID NO: 3003 | SEQ ID NO: 5369 |
| rs7740076 | SEQ ID NO: 638 | SEQ ID NO: 3004 | SEQ ID NO: 5370 |
| rs10885 | SEQ ID NO: 639 | SEQ ID NO: 3005 | SEQ ID NO: 5371 |
| rs1042149 | SEQ ID NO: 640 | SEQ ID NO: 3006 | SEQ ID NO: 5372 |
| rs3129859 | SEQ ID NO: 641 | SEQ ID NO: 3007 | SEQ ID NO: 5373 |
| rs362546 | SEQ ID NO: 642 | SEQ ID NO: 3008 | SEQ ID NO: 5374 |
| rs1633057 | SEQ ID NO: 643 | SEQ ID NO: 3009 | SEQ ID NO: 5375 |
| rs3807032 | SEQ ID NO: 644 | SEQ ID NO: 3010 | SEQ ID NO: 5376 |
| rs3130212 | SEQ ID NO: 645 | SEQ ID NO: 3011 | SEQ ID NO: 5377 |
| rs423639 | SEQ ID NO: 646 | SEQ ID NO: 3012 | SEQ ID NO: 5378 |
| rs3135000 | SEQ ID NO: 647 | SEQ ID NO: 3013 | SEQ ID NO: 5379 |
| rs1264620 | SEQ ID NO: 648 | SEQ ID NO: 3014 | SEQ ID NO: 5380 |
| rs1264703 | SEQ ID NO: 649 | SEQ ID NO: 3015 | SEQ ID NO: 5381 |
| rs1465651 | SEQ ID NO: 650 | SEQ ID NO: 3016 | SEQ ID NO: 5382 |
| rs7194 | SEQ ID NO: 651 | SEQ ID NO: 3017 | SEQ ID NO: 5383 |
| rs213210 | SEQ ID NO: 652 | SEQ ID NO: 3018 | SEQ ID NO: 5384 |
| rs2256028 | SEQ ID NO: 653 | SEQ ID NO: 3019 | SEQ ID NO: 5385 |
| rs762815 | SEQ ID NO: 654 | SEQ ID NO: 3020 | SEQ ID NO: 5386 |
| rs3868542 | SEQ ID NO: 655 | SEQ ID NO: 3021 | SEQ ID NO: 5387 |
| rs5021449 | SEQ ID NO: 656 | SEQ ID NO: 3022 | SEQ ID NO: 5388 |
| rs1632880 | SEQ ID NO: 657 | SEQ ID NO: 3023 | SEQ ID NO: 5389 |
| rs7774954 | SEQ ID NO: 658 | SEQ ID NO: 3024 | SEQ ID NO: 5390 |
| rs4713520 | SEQ ID NO: 659 | SEQ ID NO: 3025 | SEQ ID NO: 5391 |
| rs6456895 | SEQ ID NO: 660 | SEQ ID NO: 3026 | SEQ ID NO: 5392 |
| rs463302 | SEQ ID NO: 661 | SEQ ID NO: 3027 | SEQ ID NO: 5393 |
| rs1265100 | SEQ ID NO: 662 | SEQ ID NO: 3028 | SEQ ID NO: 5394 |
| rs3828875 | SEQ ID NO: 663 | SEQ ID NO: 3029 | SEQ ID NO: 5395 |
| rs6917517 | SEQ ID NO: 664 | SEQ ID NO: 3030 | SEQ ID NO: 5396 |
| rs3888777 | SEQ ID NO: 665 | SEQ ID NO: 3031 | SEQ ID NO: 5397 |
| rs7769979 | SEQ ID NO: 666 | SEQ ID NO: 3032 | SEQ ID NO: 5398 |
| rs3129931 | SEQ ID NO: 667 | SEQ ID NO: 3033 | SEQ ID NO: 5399 |
| rs3868086 | SEQ ID NO: 668 | SEQ ID NO: 3034 | SEQ ID NO: 5400 |
| rs7773068 | SEQ ID NO: 669 | SEQ ID NO: 3035 | SEQ ID NO: 5401 |
| rs3778638 | SEQ ID NO: 670 | SEQ ID NO: 3036 | SEQ ID NO: 5402 |
| rs3130843 | SEQ ID NO: 671 | SEQ ID NO: 3037 | SEQ ID NO: 5403 |
| rs3885028 | SEQ ID NO: 672 | SEQ ID NO: 3038 | SEQ ID NO: 5404 |
| rs3757327 | SEQ ID NO: 673 | SEQ ID NO: 3039 | SEQ ID NO: 5405 |
| rs29225 | SEQ ID NO: 674 | SEQ ID NO: 3040 | SEQ ID NO: 5406 |
| rs885937 | SEQ ID NO: 675 | SEQ ID NO: 3041 | SEQ ID NO: 5407 |
| rs3129702 | SEQ ID NO: 676 | SEQ ID NO: 3042 | SEQ ID NO: 5408 |
| rs3128968 | SEQ ID NO: 677 | SEQ ID NO: 3043 | SEQ ID NO: 5409 |
| rs4713327 | SEQ ID NO: 678 | SEQ ID NO: 3044 | SEQ ID NO: 5410 |
| rs987870 | SEQ ID NO: 679 | SEQ ID NO: 3045 | SEQ ID NO: 5411 |
| rs7772410 | SEQ ID NO: 680 | SEQ ID NO: 3046 | SEQ ID NO: 5412 |
| rs3998790 | SEQ ID NO: 681 | SEQ ID NO: 3047 | SEQ ID NO: 5413 |
| rs733841 | SEQ ID NO: 682 | SEQ ID NO: 3048 | SEQ ID NO: 5414 |
| rs6916716 | SEQ ID NO: 683 | SEQ ID NO: 3049 | SEQ ID NO: 5415 |
| rs2395313 | SEQ ID NO: 684 | SEQ ID NO: 3050 | SEQ ID NO: 5416 |
| rs774400 | SEQ ID NO: 685 | SEQ ID NO: 3051 | SEQ ID NO: 5417 |
| rs3757329 | SEQ ID NO: 686 | SEQ ID NO: 3052 | SEQ ID NO: 5418 |
| rs6933135 | SEQ ID NO: 687 | SEQ ID NO: 3053 | SEQ ID NO: 5419 |
| rs4713217 | SEQ ID NO: 688 | SEQ ID NO: 3054 | SEQ ID NO: 5420 |
| rs3130198 | SEQ ID NO: 689 | SEQ ID NO: 3055 | SEQ ID NO: 5421 |
| rs5027459 | SEQ ID NO: 690 | SEQ ID NO: 3056 | SEQ ID NO: 5422 |
| rs2747430 | SEQ ID NO: 691 | SEQ ID NO: 3057 | SEQ ID NO: 5423 |
| rs4248158 | SEQ ID NO: 692 | SEQ ID NO: 3058 | SEQ ID NO: 5424 |
| rs2301227 | SEQ ID NO: 693 | SEQ ID NO: 3059 | SEQ ID NO: 5425 |
| rs3668 | SEQ ID NO: 694 | SEQ ID NO: 3060 | SEQ ID NO: 5426 |
| rs1233426 | SEQ ID NO: 695 | SEQ ID NO: 3061 | SEQ ID NO: 5427 |
| rs3094093 | SEQ ID NO: 696 | SEQ ID NO: 3062 | SEQ ID NO: 5428 |
| rs422951 | SEQ ID NO: 697 | SEQ ID NO: 3063 | SEQ ID NO: 5429 |
| rs909729 | SEQ ID NO: 698 | SEQ ID NO: 3064 | SEQ ID NO: 5430 |
| rs2534673 | SEQ ID NO: 699 | SEQ ID NO: 3065 | SEQ ID NO: 5431 |
| rs6928482 | SEQ ID NO: 700 | SEQ ID NO: 3066 | SEQ ID NO: 5432 |
| rs707938 | SEQ ID NO: 701 | SEQ ID NO: 3067 | SEQ ID NO: 5433 |
| rs2844521 | SEQ ID NO: 702 | SEQ ID NO: 3068 | SEQ ID NO: 5434 |
| rs3 868075 | SEQ ID NO: 703 | SEQ ID NO: 3069 | SEQ ID NO: 5435 |
| rs7766843 | SEQ ID NO: 704 | SEQ ID NO: 3070 | SEQ ID NO: 5436 |
| rs4151651 | SEQ ID NO: 705 | SEQ ID NO: 3071 | SEQ ID NO: 5437 |
| rs2394609 | SEQ ID NO: 706 | SEQ ID NO: 3072 | SEQ ID NO: 5438 |
| rs2239805 | SEQ ID NO: 707 | SEQ ID NO: 3073 | SEQ ID NO: 5439 |
| rs5007260 | SEQ ID NO: 708 | SEQ ID NO: 3074 | SEQ ID NO: 5440 |
| rs2073044 | SEQ ID NO: 709 | SEQ ID NO: 3075 | SEQ ID NO: 5441 |
| rs3765608 | SEQ ID NO: 710 | SEQ ID NO: 3076 | SEQ ID NO: 5442 |
| rs769176 | SEQ ID NO: 711 | SEQ ID NO: 3077 | SEQ ID NO: 5443 |
| rs7773694 | SEQ ID NO: 712 | SEQ ID NO: 3078 | SEQ ID NO: 5444 |
| rs3128935 | SEQ ID NO: 713 | SEQ ID NO: 3079 | SEQ ID NO: 5445 |
| rs2853950 | SEQ ID NO: 714 | SEQ ID NO: 3080 | SEQ ID NO: 5446 |
| rs494620 | SEQ ID NO: 715 | SEQ ID NO: 3081 | SEQ ID NO: 5447 |
| rs1633089 | SEQ ID NO: 716 | SEQ ID NO: 3082 | SEQ ID NO: 5448 |
| rs2267642 | SEQ ID NO: 717 | SEQ ID NO: 3083 | SEQ ID NO: 5449 |
| rs2259571 | SEQ ID NO: 718 | SEQ ID NO: 3084 | SEQ ID NO: 5450 |
| rs1557608 | SEQ ID NO: 719 | SEQ ID NO: 3085 | SEQ ID NO: 5451 |
| rs2530708 | SEQ ID NO: 720 | SEQ ID NO: 3086 | SEQ ID NO: 5452 |
| rs7769930 | SEQ ID NO: 721 | SEQ ID NO: 3087 | SEQ ID NO: 5453 |
| rs2097274 | SEQ ID NO: 722 | SEQ ID NO: 3088 | SEQ ID NO: 5454 |
| rs422878 | SEQ ID NO: 723 | SEQ ID NO: 3089 | SEQ ID NO: 5455 |
| rs6457643 | SEQ ID NO: 724 | SEQ ID NO: 3090 | SEQ ID NO: 5456 |
| rs7381617 | SEQ ID NO: 725 | SEQ ID NO: 3091 | SEQ ID NO: 5457 |
| rs3129270 | SEQ ID NO: 726 | SEQ ID NO: 3092 | SEQ ID NO: 5458 |
| rs1632914 | SEQ ID NO: 727 | SEQ ID NO: 3093 | SEQ ID NO: 5459 |
| rs3129808 | SEQ ID NO: 728 | SEQ ID NO: 3094 | SEQ ID NO: 5460 |
| rs4713604 | SEQ ID NO: 729 | SEQ ID NO: 3095 | SEQ ID NO: 5461 |
| rs241436 | SEQ ID NO: 730 | SEQ ID NO: 3096 | SEQ ID NO: 5462 |
| rs3132555 | SEQ ID NO: 731 | SEQ ID NO: 3097 | SEQ ID NO: 5463 |
| rs2517461 | SEQ ID NO: 732 | SEQ ID NO: 3098 | SEQ ID NO: 5464 |
| rs5021448 | SEQ ID NO: 733 | SEQ ID NO: 3099 | SEQ ID NO: 5465 |
| rs4122198 | SEQ ID NO: 734 | SEQ ID NO: 3100 | SEQ ID NO: 5466 |
| rs2227134 | SEQ ID NO: 735 | SEQ ID NO: 3101 | SEQ ID NO: 5467 |
| rs4713605 | SEQ ID NO: 736 | SEQ ID NO: 3102 | SEQ ID NO: 5468 |
| rs1265158 | SEQ ID NO: 737 | SEQ ID NO: 3103 | SEQ ID NO: 5469 |
| rs3135382 | SEQ ID NO: 738 | SEQ ID NO: 3104 | SEQ ID NO: 5470 |
| rs3129701 | SEQ ID NO: 739 | SEQ ID NO: 3105 | SEQ ID NO: 5471 |
| rs2244546 | SEQ ID NO: 740 | SEQ ID NO: 3106 | SEQ ID NO: 5472 |
| rs2395175 | SEQ ID NO: 741 | SEQ ID NO: 3107 | SEQ ID NO: 5473 |
| rs2844793 | SEQ ID NO: 742 | SEQ ID NO: 3108 | SEQ ID NO: 5474 |
| rs2395327 | SEQ ID NO: 743 | SEQ ID NO: 3109 | SEQ ID NO: 5475 |
| rs1265074 | SEQ ID NO: 744 | SEQ ID NO: 3110 | SEQ ID NO: 5476 |
| rs3117228 | SEQ ID NO: 745 | SEQ ID NO: 3111 | SEQ ID NO: 5477 |
| rs429479 | SEQ ID NO: 746 | SEQ ID NO: 3112 | SEQ ID NO: 5478 |
| rs1634720 | SEQ ID NO: 747 | SEQ ID NO: 3113 | SEQ ID NO: 5479 |
| rs3095302 | SEQ ID NO: 748 | SEQ ID NO: 3114 | SEQ ID NO: 5480 |
| rs423023 | SEQ ID NO: 749 | SEQ ID NO: 3115 | SEQ ID NO: 5481 |
| rs6456880 | SEQ ID NO: 750 | SEQ ID NO: 3116 | SEQ ID NO: 5482 |
| rs1065461 | SEQ ID NO: 751 | SEQ ID NO: 3117 | SEQ ID NO: 5483 |
| rs6923313 | SEQ ID NO: 752 | SEQ ID NO: 3118 | SEQ ID NO: 5484 |
| rs1367730 | SEQ ID NO: 753 | SEQ ID NO: 3119 | SEQ ID NO: 5485 |
| rs1800629 | SEQ ID NO: 754 | SEQ ID NO: 3120 | SEQ ID NO: 5486 |
| rs2240070 | SEQ ID NO: 755 | SEQ ID NO: 3121 | SEQ ID NO: 5487 |
| rs520803 | SEQ ID NO: 756 | SEQ ID NO: 3122 | SEQ ID NO: 5488 |
| rs3734836 | SEQ ID NO: 757 | SEQ ID NO: 3123 | SEQ ID NO: 5489 |
| rs3132468 | SEQ ID NO: 758 | SEQ ID NO: 3124 | SEQ ID NO: 5490 |
| rs3094159 | SEQ ID NO: 759 | SEQ ID NO: 3125 | SEQ ID NO: 5491 |
| rs3180665 | SEQ ID NO: 760 | SEQ ID NO: 3126 | SEQ ID NO: 5492 |
| rs2518027 | SEQ ID NO: 761 | SEQ ID NO: 3127 | SEQ ID NO: 5493 |
| rs1362068 | SEQ ID NO: 762 | SEQ ID NO: 3128 | SEQ ID NO: 5494 |
| rs1264542 | SEQ ID NO: 763 | SEQ ID NO: 3129 | SEQ ID NO: 5495 |
| rs2223638 | SEQ ID NO: 764 | SEQ ID NO: 3130 | SEQ ID NO: 5496 |
| rs2281390 | SEQ ID NO: 765 | SEQ ID NO: 3131 | SEQ ID NO: 5497 |
| rs1265094 | SEQ ID NO: 766 | SEQ ID NO: 3132 | SEQ ID NO: 5498 |
| rs2523994 | SEQ ID NO: 767 | SEQ ID NO: 3133 | SEQ ID NO: 5499 |
| rs2857129 | SEQ ID NO: 768 | SEQ ID NO: 3134 | SEQ ID NO: 5500 |
| rs886403 | SEQ ID NO: 769 | SEQ ID NO: 3135 | SEQ ID NO: 5501 |
| rs2213565 | SEQ ID NO: 770 | SEQ ID NO: 3136 | SEQ ID NO: 5502 |
| rs1419658 | SEQ ID NO: 771 | SEQ ID NO: 3137 | SEQ ID NO: 5503 |
| rs2022109 | SEQ ID NO: 772 | SEQ ID NO: 3138 | SEQ ID NO: 5504 |
| rs3128928 | SEQ ID NO: 773 | SEQ ID NO: 3139 | SEQ ID NO: 5505 |
| rs707913 | SEQ ID NO: 774 | SEQ ID NO: 3140 | SEQ ID NO: 5506 |
| rs2027856 | SEQ ID NO: 775 | SEQ ID NO: 3141 | SEQ ID NO: 5507 |
| rs2747454 | SEQ ID NO: 776 | SEQ ID NO: 3142 | SEQ ID NO: 5508 |
| rs3132552 | SEQ ID NO: 777 | SEQ ID NO: 3143 | SEQ ID NO: 5509 |
| rs3130989 | SEQ ID NO: 778 | SEQ ID NO: 3144 | SEQ ID NO: 5510 |
| rs3817964 | SEQ ID NO: 779 | SEQ ID NO: 3145 | SEQ ID NO: 5511 |
| rs154971 | SEQ ID NO: 780 | SEQ ID NO: 3146 | SEQ ID NO: 5512 |
| rs4713326 | SEQ ID NO: 781 | SEQ ID NO: 3147 | SEQ ID NO: 5513 |
| rs3132499 | SEQ ID NO: 782 | SEQ ID NO: 3148 | SEQ ID NO: 5514 |
| rs2395318 | SEQ ID NO: 783 | SEQ ID NO: 3149 | SEQ ID NO: 5515 |
| rs3823343 | SEQ ID NO: 784 | SEQ ID NO: 3150 | SEQ ID NO: 5516 |
| rs2071463 | SEQ ID NO: 785 | SEQ ID NO: 3151 | SEQ ID NO: 5517 |
| rs2857770 | SEQ ID NO: 786 | SEQ ID NO: 3152 | SEQ ID NO: 5518 |
| rs4248154 | SEQ ID NO: 787 | SEQ ID NO: 3153 | SEQ ID NO: 5519 |
| rs3132595 | SEQ ID NO: 788 | SEQ ID NO: 3154 | SEQ ID NO: 5520 |
| rs3749970 | SEQ ID NO: 789 | SEQ ID NO: 3155 | SEQ ID NO: 5521 |
| rs541862 | SEQ ID NO: 790 | SEQ ID NO: 3156 | SEQ ID NO: 5522 |
| rs2075582 | SEQ ID NO: 791 | SEQ ID NO: 3157 | SEQ ID NO: 5523 |
| rs2854048 | SEQ ID NO: 792 | SEQ ID NO: 3158 | SEQ ID NO: 5524 |
| rs1557607 | SEQ ID NO: 793 | SEQ ID NO: 3159 | SEQ ID NO: 5525 |
| rs1362070 | SEQ ID NO: 794 | SEQ ID NO: 3160 | SEQ ID NO: 5526 |
| rs2621337 | SEQ ID NO: 795 | SEQ ID NO: 3161 | SEQ ID NO: 5527 |
| rs3807030 | SEQ ID NO: 796 | SEQ ID NO: 3162 | SEQ ID NO: 5528 |
| rs2596502 | SEQ ID NO: 797 | SEQ ID NO: 3163 | SEQ ID NO: 5529 |
| rs2076530 | SEQ ID NO: 798 | SEQ ID NO: 3164 | SEQ ID NO: 5530 |
| rs885932 | SEQ ID NO: 799 | SEQ ID NO: 3165 | SEQ ID NO: 5531 |
| rs2517598 | SEQ ID NO: 800 | SEQ ID NO: 3166 | SEQ ID NO: 5532 |
| rs2524079 | SEQ ID NO: 801 | SEQ ID NO: 3167 | SEQ ID NO: 5533 |
| rs4286818 | SEQ ID NO: 802 | SEQ ID NO: 3168 | SEQ ID NO: 5534 |
| rs4151650 | SEQ ID NO: 803 | SEQ ID NO: 3169 | SEQ ID NO: 5535 |
| rs2523933 | SEQ ID NO: 804 | SEQ ID NO: 3170 | SEQ ID NO: 5536 |
| rs194675 | SEQ ID NO: 805 | SEQ ID NO: 3171 | SEQ ID NO: 5537 |
| rs3130615 | SEQ ID NO: 806 | SEQ ID NO: 3172 | SEQ ID NO: 5538 |
| rs2050188 | SEQ ID NO: 807 | SEQ ID NO: 3173 | SEQ ID NO: 5539 |
| rs2395173 | SEQ ID NO: 808 | SEQ ID NO: 3174 | SEQ ID NO: 5540 |
| rs1573299 | SEQ ID NO: 809 | SEQ ID NO: 3175 | SEQ ID NO: 5541 |
| rs417812 | SEQ ID NO: 810 | SEQ ID NO: 3176 | SEQ ID NO: 5542 |
| rs1633029 | SEQ ID NO: 811 | SEQ ID NO: 3177 | SEQ ID NO: 5543 |
| rs7454158 | SEQ ID NO: 812 | SEQ ID NO: 3178 | SEQ ID NO: 5544 |
| rs2516670 | SEQ ID NO: 813 | SEQ ID NO: 3179 | SEQ ID NO: 5545 |
| rs4151652 | SEQ ID NO: 814 | SEQ ID NO: 3180 | SEQ ID NO: 5546 |
| rs5026741 | SEQ ID NO: 815 | SEQ ID NO: 3181 | SEQ ID NO: 5547 |
| rs2158286 | SEQ ID NO: 816 | SEQ ID NO: 3182 | SEQ ID NO: 5548 |
| rs1051790 | SEQ ID NO: 817 | SEQ ID NO: 3183 | SEQ ID NO: 5549 |
| rs3828799 | SEQ ID NO: 818 | SEQ ID NO: 3184 | SEQ ID NO: 5550 |
| rs1737005 | SEQ ID NO: 819 | SEQ ID NO: 3185 | SEQ ID NO: 5551 |
| rs3132946 | SEQ ID NO: 820 | SEQ ID NO: 3186 | SEQ ID NO: 5552 |
| rs3132467 | SEQ ID NO: 821 | SEQ ID NO: 3187 | SEQ ID NO: 5553 |
| rs2076542 | SEQ ID NO: 822 | SEQ ID NO: 3188 | SEQ ID NO: 5554 |
| rs3828882 | SEQ ID NO: 823 | SEQ ID NO: 3189 | SEQ ID NO: 5555 |
| rs3094717 | SEQ ID NO: 824 | SEQ ID NO: 3190 | SEQ ID NO: 5556 |
| rs3094214 | SEQ ID NO: 825 | SEQ ID NO: 3191 | SEQ ID NO: 5557 |
| rs2734984 | SEQ ID NO: 826 | SEQ ID NO: 3192 | SEQ ID NO: 5558 |
| rs3131018 | SEQ ID NO: 827 | SEQ ID NO: 3193 | SEQ ID NO: 5559 |
| rs419957 | SEQ ID NO: 828 | SEQ ID NO: 3194 | SEQ ID NO: 5560 |
| rs1737075 | SEQ ID NO: 829 | SEQ ID NO: 3195 | SEQ ID NO: 5561 |
| rs3765607 | SEQ ID NO: 830 | SEQ ID NO: 3196 | SEQ ID NO: 5562 |
| rs1800683 | SEQ ID NO: 831 | SEQ ID NO: 3197 | SEQ ID NO: 5563 |
| rs3763315 | SEQ ID NO: 832 | SEQ ID NO: 3198 | SEQ ID NO: 5564 |
| rs2187824 | SEQ ID NO: 833 | SEQ ID NO: 3199 | SEQ ID NO: 5565 |
| rs6457041 | SEQ ID NO: 834 | SEQ ID NO: 3200 | SEQ ID NO: 5566 |
| rs2285797 | SEQ ID NO: 835 | SEQ ID NO: 3201 | SEQ ID NO: 5567 |
| rs2621336 | SEQ ID NO: 836 | SEQ ID NO: 3202 | SEQ ID NO: 5568 |
| rs4713614 | SEQ ID NO: 837 | SEQ ID NO: 3203 | SEQ ID NO: 5569 |
| rs4428528 | SEQ ID NO: 838 | SEQ ID NO: 3204 | SEQ ID NO: 5570 |
| rs2071365 | SEQ ID NO: 839 | SEQ ID NO: 3205 | SEQ ID NO: 5571 |
| rs2246638 | SEQ ID NO: 840 | SEQ ID NO: 3206 | SEQ ID NO: 5572 |
| rs1264697 | SEQ ID NO: 841 | SEQ ID NO: 3207 | SEQ ID NO: 5573 |
| rs2280773 | SEQ ID NO: 842 | SEQ ID NO: 3208 | SEQ ID NO: 5574 |
| rs4248169 | SEQ ID NO: 843 | SEQ ID NO: 3209 | SEQ ID NO: 5575 |
| rs1632941 | SEQ ID NO: 844 | SEQ ID NO: 3210 | SEQ ID NO: 5576 |
| rs1610653 | SEQ ID NO: 845 | SEQ ID NO: 3211 | SEQ ID NO: 5577 |
| rs6911894 | SEQ ID NO: 846 | SEQ ID NO: 3212 | SEQ ID NO: 5578 |
| rs3778614 | SEQ ID NO: 847 | SEQ ID NO: 3213 | SEQ ID NO: 5579 |
| rs2523619 | SEQ ID NO: 848 | SEQ ID NO: 3214 | SEQ ID NO: 5580 |
| rs3213483 | SEQ ID NO: 849 | SEQ ID NO: 3215 | SEQ ID NO: 5581 |
| rs2857713 | SEQ ID NO: 850 | SEQ ID NO: 3216 | SEQ ID NO: 5582 |
| rs2072632 | SEQ ID NO: 851 | SEQ ID NO: 3217 | SEQ ID NO: 5583 |
| rs2074469 | SEQ ID NO: 852 | SEQ ID NO: 3218 | SEQ ID NO: 5584 |
| rs2071876 | SEQ ID NO: 853 | SEQ ID NO: 3219 | SEQ ID NO: 5585 |
| rs3819686 | SEQ ID NO: 854 | SEQ ID NO: 3220 | SEQ ID NO: 5586 |
| rs1049414 | SEQ ID NO: 855 | SEQ ID NO: 3221 | SEQ ID NO: 5587 |
| rs1048412 | SEQ ID NO: 856 | SEQ ID NO: 3222 | SEQ ID NO: 5588 |
| rs2269420 | SEQ ID NO: 857 | SEQ ID NO: 3223 | SEQ ID NO: 5589 |
| rs7750641 | SEQ ID NO: 858 | SEQ ID NO: 3224 | SEQ ID NO: 5590 |
| rs2647027 | SEQ ID NO: 859 | SEQ ID NO: 3225 | SEQ ID NO: 5591 |
| rs3815095 | SEQ ID NO: 860 | SEQ ID NO: 3226 | SEQ ID NO: 5592 |
| rs3130999 | SEQ ID NO: 861 | SEQ ID NO: 3227 | SEQ ID NO: 5593 |
| rs3134940 | SEQ ID NO: 862 | SEQ ID NO: 3228 | SEQ ID NO: 5594 |
| rs743400 | SEQ ID NO: 863 | SEQ ID NO: 3229 | SEQ ID NO: 5595 |
| rs4711187 | SEQ ID NO: 864 | SEQ ID NO: 3230 | SEQ ID NO: 5596 |
| rs2395038 | SEQ ID NO: 865 | SEQ ID NO: 3231 | SEQ ID NO: 5597 |
| rs2239689 | SEQ ID NO: 866 | SEQ ID NO: 3232 | SEQ ID NO: 5598 |
| rs995185 | SEQ ID NO: 867 | SEQ ID NO: 3233 | SEQ ID NO: 5599 |
| rs3830041 | SEQ ID NO: 868 | SEQ ID NO: 3234 | SEQ ID NO: 5600 |
| rs2523945 | SEQ ID NO: 869 | SEQ ID NO: 3235 | SEQ ID NO: 5601 |
| rs3130099 | SEQ ID NO: 870 | SEQ ID NO: 3236 | SEQ ID NO: 5602 |
| rs3130374 | SEQ ID NO: 871 | SEQ ID NO: 3237 | SEQ ID NO: 5603 |
| rs2524272 | SEQ ID NO: 872 | SEQ ID NO: 3238 | SEQ ID NO: 5604 |
| rs3093558 | SEQ ID NO: 873 | SEQ ID NO: 3239 | SEQ ID NO: 5605 |
| rs7656 | SEQ ID NO: 874 | SEQ ID NO: 3240 | SEQ ID NO: 5606 |
| rs3093992 | SEQ ID NO: 875 | SEQ ID NO: 3241 | SEQ ID NO: 5607 |
| rs2428509 | SEQ ID NO: 876 | SEQ ID NO: 3242 | SEQ ID NO: 5608 |
| rs2071292 | SEQ ID NO: 877 | SEQ ID NO: 3243 | SEQ ID NO: 5609 |
| rs107822 | SEQ ID NO: 878 | SEQ ID NO: 3244 | SEQ ID NO: 5610 |
| rs3734860 | SEQ ID NO: 879 | SEQ ID NO: 3245 | SEQ ID NO: 5611 |
| rs4151667 | SEQ ID NO: 880 | SEQ ID NO: 3246 | SEQ ID NO: 5612 |
| rs887466 | SEQ ID NO: 881 | SEQ ID NO: 3247 | SEQ ID NO: 5613 |
| rs2857096 | SEQ ID NO: 882 | SEQ ID NO: 3248 | SEQ ID NO: 5614 |
| rs3130976 | SEQ ID NO: 883 | SEQ ID NO: 3249 | SEQ ID NO: 5615 |
| rs6910473 | SEQ ID NO: 884 | SEQ ID NO: 3250 | SEQ ID NO: 5616 |
| rs1633012 | SEQ ID NO: 885 | SEQ ID NO: 3251 | SEQ ID NO: 5617 |
| rs2523399 | SEQ ID NO: 886 | SEQ ID NO: 3252 | SEQ ID NO: 5618 |
| rs2071786 | SEQ ID NO: 887 | SEQ ID NO: 3253 | SEQ ID NO: 5619 |
| rs3823359 | SEQ ID NO: 888 | SEQ ID NO: 3254 | SEQ ID NO: 5620 |
| rs400488 | SEQ ID NO: 889 | SEQ ID NO: 3255 | SEQ ID NO: 5621 |
| rs2239697 | SEQ ID NO: 890 | SEQ ID NO: 3256 | SEQ ID NO: 5622 |
| rs4959041 | SEQ ID NO: 891 | SEQ ID NO: 3257 | SEQ ID NO: 5623 |
| rs1042434 | SEQ ID NO: 892 | SEQ ID NO: 3258 | SEQ ID NO: 5624 |
| rs3819275 | SEQ ID NO: 893 | SEQ ID NO: 3259 | SEQ ID NO: 5625 |
| rs381218 | SEQ ID NO: 894 | SEQ ID NO: 3260 | SEQ ID NO: 5626 |
| rs29230 | SEQ ID NO: 895 | SEQ ID NO: 3261 | SEQ ID NO: 5627 |
| rs2301226 | SEQ ID NO: 896 | SEQ ID NO: 3262 | SEQ ID NO: 5628 |
| rs742870 | SEQ ID NO: 897 | SEQ ID NO: 3263 | SEQ ID NO: 5629 |
| rs6905862 | SEQ ID NO: 898 | SEQ ID NO: 3264 | SEQ ID NO: 5630 |
| rs3129938 | SEQ ID NO: 899 | SEQ ID NO: 3265 | SEQ ID NO: 5631 |
| rs1736939 | SEQ ID NO: 900 | SEQ ID NO: 3266 | SEQ ID NO: 5632 |
| rs4548006 | SEQ ID NO: 901 | SEQ ID NO: 3267 | SEQ ID NO: 5633 |
| rs2857767 | SEQ ID NO: 902 | SEQ ID NO: 3268 | SEQ ID NO: 5634 |
| rs241442 | SEQ ID NO: 903 | SEQ ID NO: 3269 | SEQ ID NO: 5635 |
| rs3093671 | SEQ ID NO: 904 | SEQ ID NO: 3270 | SEQ ID NO: 5636 |
| rs2857433 | SEQ ID NO: 905 | SEQ ID NO: 3271 | SEQ ID NO: 5637 |
| rs1800453 | SEQ ID NO: 906 | SEQ ID NO: 3272 | SEQ ID NO: 5638 |
| rs1049628 | SEQ ID NO: 907 | SEQ ID NO: 3273 | SEQ ID NO: 5639 |
| rs2769 | SEQ ID NO: 908 | SEQ ID NO: 3274 | SEQ ID NO: 5640 |
| rs3763295 | SEQ ID NO: 909 | SEQ ID NO: 3275 | SEQ ID NO: 5641 |
| rs3763338 | SEQ ID NO: 910 | SEQ ID NO: 3276 | SEQ ID NO: 5642 |
| rs6910733 | SEQ ID NO: 911 | SEQ ID NO: 3277 | SEQ ID NO: 5643 |
| rs3830037 | SEQ ID NO: 912 | SEQ ID NO: 3278 | SEQ ID NO: 5644 |
| rs2858330 | SEQ ID NO: 913 | SEQ ID NO: 3279 | SEQ ID NO: 5645 |
| rs1076711 | SEQ ID NO: 914 | SEQ ID NO: 3280 | SEQ ID NO: 5646 |
| rs2272874 | SEQ ID NO: 915 | SEQ ID NO: 3281 | SEQ ID NO: 5647 |
| rs762324 | SEQ ID NO: 916 | SEQ ID NO: 3282 | SEQ ID NO: 5648 |
| rs2061952 | SEQ ID NO: 917 | SEQ ID NO: 3283 | SEQ ID NO: 5649 |
| rs4647195 | SEQ ID NO: 918 | SEQ ID NO: 3284 | SEQ ID NO: 5650 |
| rs2523400 | SEQ ID NO: 919 | SEQ ID NO: 3285 | SEQ ID NO: 5651 |
| rs1041981 | SEQ ID NO: 920 | SEQ ID NO: 3286 | SEQ ID NO: 5652 |
| rs1367729 | SEQ ID NO: 921 | SEQ ID NO: 3287 | SEQ ID NO: 5653 |
| rs2523943 | SEQ ID NO: 922 | SEQ ID NO: 3288 | SEQ ID NO: 5654 |
| rs1233421 | SEQ ID NO: 923 | SEQ ID NO: 3289 | SEQ ID NO: 5655 |
| rs4988888 | SEQ ID NO: 924 | SEQ ID NO: 3290 | SEQ ID NO: 5656 |
| rs4713260 | SEQ ID NO: 925 | SEQ ID NO: 3291 | SEQ ID NO: 5657 |
| rs3095227 | SEQ ID NO: 926 | SEQ ID NO: 3292 | SEQ ID NO: 5658 |
| rs2596538 | SEQ ID NO: 927 | SEQ ID NO: 3293 | SEQ ID NO: 5659 |
| rs1480382 | SEQ ID NO: 928 | SEQ ID NO: 3294 | SEQ ID NO: 5660 |
| rs2229699 | SEQ ID NO: 929 | SEQ ID NO: 3295 | SEQ ID NO: 5661 |
| rs2276447 | SEQ ID NO: 930 | SEQ ID NO: 3296 | SEQ ID NO: 5662 |
| rs2074511 | SEQ ID NO: 931 | SEQ ID NO: 3297 | SEQ ID NO: 5663 |
| rs2285795 | SEQ ID NO: 932 | SEQ ID NO: 3298 | SEQ ID NO: 5664 |
| rs2523959 | SEQ ID NO: 933 | SEQ ID NO: 3299 | SEQ ID NO: 5665 |
| rs5020983 | SEQ ID NO: 934 | SEQ ID NO: 3300 | SEQ ID NO: 5666 |
| rs2071353 | SEQ ID NO: 935 | SEQ ID NO: 3301 | SEQ ID NO: 5667 |
| rs1610646 | SEQ ID NO: 936 | SEQ ID NO: 3302 | SEQ ID NO: 5668 |
| rs3117035 | SEQ ID NO: 937 | SEQ ID NO: 3303 | SEQ ID NO: 5669 |
| rs2071470 | SEQ ID NO: 938 | SEQ ID NO: 3304 | SEQ ID NO: 5670 |
| rs376892 | SEQ ID NO: 939 | SEQ ID NO: 3305 | SEQ ID NO: 5671 |
| rs1631950 | SEQ ID NO: 940 | SEQ ID NO: 3306 | SEQ ID NO: 5672 |
| rs206781 | SEQ ID NO: 941 | SEQ ID NO: 3307 | SEQ ID NO: 5673 |
| rs2071592 | SEQ ID NO: 942 | SEQ ID NO: 3308 | SEQ ID NO: 5674 |
| rs4713274 | SEQ ID NO: 943 | SEQ ID NO: 3309 | SEQ ID NO: 5675 |
| rs4959119 | SEQ ID NO: 944 | SEQ ID NO: 3310 | SEQ ID NO: 5676 |
| rs3129744 | SEQ ID NO: 945 | SEQ ID NO: 3311 | SEQ ID NO: 5677 |
| rs1737086 | SEQ ID NO: 946 | SEQ ID NO: 3312 | SEQ ID NO: 5678 |
| rs2523960 | SEQ ID NO: 947 | SEQ ID NO: 3313 | SEQ ID NO: 5679 |
| rs5020984 | SEQ ID NO: 948 | SEQ ID NO: 3314 | SEQ ID NO: 5680 |
| rs3093542 | SEQ ID NO: 949 | SEQ ID NO: 3315 | SEQ ID NO: 5681 |
| rs2127680 | SEQ ID NO: 950 | SEQ ID NO: 3316 | SEQ ID NO: 5682 |
| rs2844508 | SEQ ID NO: 951 | SEQ ID NO: 3317 | SEQ ID NO: 5683 |
| rs3093554 | SEQ ID NO: 952 | SEQ ID NO: 3318 | SEQ ID NO: 5684 |
| rs1268928 | SEQ ID NO: 953 | SEQ ID NO: 3319 | SEQ ID NO: 5685 |
| rs2294881 | SEQ ID NO: 954 | SEQ ID NO: 3320 | SEQ ID NO: 5686 |
| rs7773149 | SEQ ID NO: 955 | SEQ ID NO: 3321 | SEQ ID NO: 5687 |
| rs2524197 | SEQ ID NO: 956 | SEQ ID NO: 3322 | SEQ ID NO: 5688 |
| rs1419635 | SEQ ID NO: 957 | SEQ ID NO: 3323 | SEQ ID NO: 5689 |
| rs7755774 | SEQ ID NO: 958 | SEQ ID NO: 3324 | SEQ ID NO: 5690 |
| rs3131001 | SEQ ID NO: 959 | SEQ ID NO: 3325 | SEQ ID NO: 5691 |
| rs1610676 | SEQ ID NO: 960 | SEQ ID NO: 3326 | SEQ ID NO: 5692 |
| rs6901686 | SEQ ID NO: 961 | SEQ ID NO: 3327 | SEQ ID NO: 5693 |
| rs3094200 | SEQ ID NO: 962 | SEQ ID NO: 3328 | SEQ ID NO: 5694 |
| rs2523705 | SEQ ID NO: 963 | SEQ ID NO: 3329 | SEQ ID NO: 5695 |
| rs885948 | SEQ ID NO: 964 | SEQ ID NO: 3330 | SEQ ID NO: 5696 |
| rs2009658 | SEQ ID NO: 965 | SEQ ID NO: 3331 | SEQ ID NO: 5697 |
| rs2517754 | SEQ ID NO: 966 | SEQ ID NO: 3332 | SEQ ID NO: 5698 |
| rs2596499 | SEQ ID NO: 967 | SEQ ID NO: 3333 | SEQ ID NO: 5699 |
| rs929156 | SEQ ID NO: 968 | SEQ ID NO: 3334 | SEQ ID NO: 5700 |
| rs2076312 | SEQ ID NO: 969 | SEQ ID NO: 3335 | SEQ ID NO: 5701 |
| rs3093666 | SEQ ID NO: 970 | SEQ ID NO: 3336 | SEQ ID NO: 5702 |
| rs3132520 | SEQ ID NO: 971 | SEQ ID NO: 3337 | SEQ ID NO: 5703 |
| rs7773175 | SEQ ID NO: 972 | SEQ ID NO: 3338 | SEQ ID NO: 5704 |
| rs3116956 | SEQ ID NO: 973 | SEQ ID NO: 3339 | SEQ ID NO: 5705 |
| rs3823417 | SEQ ID NO: 974 | SEQ ID NO: 3340 | SEQ ID NO: 5706 |
| rs1265092 | SEQ ID NO: 975 | SEQ ID NO: 3341 | SEQ ID NO: 5707 |
| rs204889 | SEQ ID NO: 976 | SEQ ID NO: 3342 | SEQ ID NO: 5708 |
| rs910320 | SEQ ID NO: 977 | SEQ ID NO: 3343 | SEQ ID NO: 5709 |
| rs2239886 | SEQ ID NO: 978 | SEQ ID NO: 3344 | SEQ ID NO: 5710 |
| rs2158279 | SEQ ID NO: 979 | SEQ ID NO: 3345 | SEQ ID NO: 5711 |
| rs463260 | SEQ ID NO: 980 | SEQ ID NO: 3346 | SEQ ID NO: 5712 |
| rs2523713 | SEQ ID NO: 981 | SEQ ID NO: 3347 | SEQ ID NO: 5713 |
| rs2072915 | SEQ ID NO: 982 | SEQ ID NO: 3348 | SEQ ID NO: 5714 |
| rs2187578 | SEQ ID NO: 983 | SEQ ID NO: 3349 | SEQ ID NO: 5715 |
| rs2246626 | SEQ ID NO: 984 | SEQ ID NO: 3350 | SEQ ID NO: 5716 |
| rs2517672 | SEQ ID NO: 985 | SEQ ID NO: 3351 | SEQ ID NO: 5717 |
| rs2076489 | SEQ ID NO: 986 | SEQ ID NO: 3352 | SEQ ID NO: 5718 |
| rs1264322 | SEQ ID NO: 987 | SEQ ID NO: 3353 | SEQ ID NO: 5719 |
| rs2523970 | SEQ ID NO: 988 | SEQ ID NO: 3354 | SEQ ID NO: 5720 |
| rs2073722 | SEQ ID NO: 989 | SEQ ID NO: 3355 | SEQ ID NO: 5721 |
| rs6915224 | SEQ ID NO: 990 | SEQ ID NO: 3356 | SEQ ID NO: 5722 |
| rs209130 | SEQ ID NO: 991 | SEQ ID NO: 3357 | SEQ ID NO: 5723 |
| rs5017092 | SEQ ID NO: 992 | SEQ ID NO: 3358 | SEQ ID NO: 5724 |
| rs3130552 | SEQ ID NO: 993 | SEQ ID NO: 3359 | SEQ ID NO: 5725 |
| rs2247720 | SEQ ID NO: 994 | SEQ ID NO: 3360 | SEQ ID NO: 5726 |
| rs3807036 | SEQ ID NO: 995 | SEQ ID NO: 3361 | SEQ ID NO: 5727 |
| rs1049622 | SEQ ID NO: 996 | SEQ ID NO: 3362 | SEQ ID NO: 5728 |
| rs2179915 | SEQ ID NO: 997 | SEQ ID NO: 3363 | SEQ ID NO: 5729 |
| rs6930217 | SEQ ID NO: 998 | SEQ ID NO: 3364 | SEQ ID NO: 5730 |
| rs3093553 | SEQ ID NO: 999 | SEQ ID NO: 3365 | SEQ ID NO: 5731 |
| rs6457597 | SEQ ID NO: 1000 | SEQ ID NO: 3366 | SEQ ID NO: 5732 |
| rs241437 | SEQ ID NO: 1001 | SEQ ID NO: 3367 | SEQ ID NO: 5733 |
| rs5026740 | SEQ ID NO: 1002 | SEQ ID NO: 3368 | SEQ ID NO: 5734 |
| rs15604 | SEQ ID NO: 1003 | SEQ ID NO: 3369 | SEQ ID NO: 5735 |
| rs2071800 | SEQ ID NO: 1004 | SEQ ID NO: 3370 | SEQ ID NO: 5736 |
| rs2844522 | SEQ ID NO: 1005 | SEQ ID NO: 3371 | SEQ ID NO: 5737 |
| rs2516409 | SEQ ID NO: 1006 | SEQ ID NO: 3372 | SEQ ID NO: 5738 |
| rs7770592 | SEQ ID NO: 1007 | SEQ ID NO: 3373 | SEQ ID NO: 5739 |
| rs444013 | SEQ ID NO: 1008 | SEQ ID NO: 3374 | SEQ ID NO: 5740 |
| rs2844846 | SEQ ID NO: 1009 | SEQ ID NO: 3375 | SEQ ID NO: 5741 |
| rs3757346 | SEQ ID NO: 1010 | SEQ ID NO: 3376 | SEQ ID NO: 5742 |
| rs3134929 | SEQ ID NO: 1011 | SEQ ID NO: 3377 | SEQ ID NO: 5743 |
| rs401775 | SEQ ID NO: 1012 | SEQ ID NO: 3378 | SEQ ID NO: 5744 |
| rs6924267 | SEQ ID NO: 1013 | SEQ ID NO: 3379 | SEQ ID NO: 5745 |
| rs2517673 | SEQ ID NO: 1014 | SEQ ID NO: 3380 | SEQ ID NO: 5746 |
| rs1736980 | SEQ ID NO: 1015 | SEQ ID NO: 3381 | SEQ ID NO: 5747 |
| rs1611203 | SEQ ID NO: 1016 | SEQ ID NO: 3382 | SEQ ID NO: 5748 |
| rs7768484 | SEQ ID NO: 1017 | SEQ ID NO: 3383 | SEQ ID NO: 5749 |
| rs7741100 | SEQ ID NO: 1018 | SEQ ID NO: 3384 | SEQ ID NO: 5750 |
| rs2294884 | SEQ ID NO: 1019 | SEQ ID NO: 3385 | SEQ ID NO: 5751 |
| rs2308935 | SEQ ID NO: 1020 | SEQ ID NO: 3386 | SEQ ID NO: 5752 |
| rs2282850 | SEQ ID NO: 1021 | SEQ ID NO: 3387 | SEQ ID NO: 5753 |
| rs2073154 | SEQ ID NO: 1022 | SEQ ID NO: 3388 | SEQ ID NO: 5754 |
| rs1265095 | SEQ ID NO: 1023 | SEQ ID NO: 3389 | SEQ ID NO: 5755 |
| rs3095317 | SEQ ID NO: 1024 | SEQ ID NO: 3390 | SEQ ID NO: 5756 |
| rs3778630 | SEQ ID NO: 1025 | SEQ ID NO: 3391 | SEQ ID NO: 5757 |
| rs3869090 | SEQ ID NO: 1026 | SEQ ID NO: 3392 | SEQ ID NO: 5758 |
| rs415929 | SEQ ID NO: 1027 | SEQ ID NO: 3393 | SEQ ID NO: 5759 |
| rs2747431 | SEQ ID NO: 1028 | SEQ ID NO: 3394 | SEQ ID NO: 5760 |
| rs2428507 | SEQ ID NO: 1029 | SEQ ID NO: 3395 | SEQ ID NO: 5761 |
| rs2295666 | SEQ ID NO: 1030 | SEQ ID NO: 3396 | SEQ ID NO: 5762 |
| rs1233427 | SEQ ID NO: 1031 | SEQ ID NO: 3397 | SEQ ID NO: 5763 |
| rs7774644 | SEQ ID NO: 1032 | SEQ ID NO: 3398 | SEQ ID NO: 5764 |
| rs3095309 | SEQ ID NO: 1033 | SEQ ID NO: 3399 | SEQ ID NO: 5765 |
| rs5872 | SEQ ID NO: 1034 | SEQ ID NO: 3400 | SEQ ID NO: 5766 |
| rs1033500 | SEQ ID NO: 1035 | SEQ ID NO: 3401 | SEQ ID NO: 5767 |
| rs3763306 | SEQ ID NO: 1036 | SEQ ID NO: 3402 | SEQ ID NO: 5768 |
| rs635688 | SEQ ID NO: 1037 | SEQ ID NO: 3403 | SEQ ID NO: 5769 |
| rs4151669 | SEQ ID NO: 1038 | SEQ ID NO: 3404 | SEQ ID NO: 5770 |
| rs2076527 | SEQ ID NO: 1039 | SEQ ID NO: 3405 | SEQ ID NO: 5771 |
| rs3178162 | SEQ ID NO: 1040 | SEQ ID NO: 3406 | SEQ ID NO: 5772 |
| rs241449 | SEQ ID NO: 1041 | SEQ ID NO: 3407 | SEQ ID NO: 5773 |
| rs4435981 | SEQ ID NO: 1042 | SEQ ID NO: 3408 | SEQ ID NO: 5774 |
| rs6456897 | SEQ ID NO: 1043 | SEQ ID NO: 3409 | SEQ ID NO: 5775 |
| rs3828923 | SEQ ID NO: 1044 | SEQ ID NO: 3410 | SEQ ID NO: 5776 |
| rs2179920 | SEQ ID NO: 1045 | SEQ ID NO: 3411 | SEQ ID NO: 5777 |
| rs3218806 | SEQ ID NO: 1046 | SEQ ID NO: 3412 | SEQ ID NO: 5778 |
| rs2076175 | SEQ ID NO: 1047 | SEQ ID NO: 3413 | SEQ ID NO: 5779 |
| rs3871246 | SEQ ID NO: 1048 | SEQ ID NO: 3414 | SEQ ID NO: 5780 |
| rs2281388 | SEQ ID NO: 1049 | SEQ ID NO: 3415 | SEQ ID NO: 5781 |
| rs3763314 | SEQ ID NO: 1050 | SEQ ID NO: 3416 | SEQ ID NO: 5782 |
| rs6913173 | SEQ ID NO: 1051 | SEQ ID NO: 3417 | SEQ ID NO: 5783 |
| rs3873408 | SEQ ID NO: 1052 | SEQ ID NO: 3418 | SEQ ID NO: 5784 |
| rs3997995 | SEQ ID NO: 1053 | SEQ ID NO: 3419 | SEQ ID NO: 5785 |
| rs2523924 | SEQ ID NO: 1054 | SEQ ID NO: 3420 | SEQ ID NO: 5786 |
| rs2239517 | SEQ ID NO: 1055 | SEQ ID NO: 3421 | SEQ ID NO: 5787 |
| rs3094225 | SEQ ID NO: 1056 | SEQ ID NO: 3422 | SEQ ID NO: 5788 |
| rs3869117 | SEQ ID NO: 1057 | SEQ ID NO: 3423 | SEQ ID NO: 5789 |
| rs3734837 | SEQ ID NO: 1058 | SEQ ID NO: 3424 | SEQ ID NO: 5790 |
| rs2524180 | SEQ ID NO: 1059 | SEQ ID NO: 3425 | SEQ ID NO: 5791 |
| rs2844804 | SEQ ID NO: 1060 | SEQ ID NO: 3426 | SEQ ID NO: 5792 |
| rs2206618 | SEQ ID NO: 1061 | SEQ ID NO: 3427 | SEQ ID NO: 5793 |
| rs406424 | SEQ ID NO: 1062 | SEQ ID NO: 3428 | SEQ ID NO: 5794 |
| rs3830038 | SEQ ID NO: 1063 | SEQ ID NO: 3429 | SEQ ID NO: 5795 |
| rs1048709 | SEQ ID NO: 1064 | SEQ ID NO: 3430 | SEQ ID NO: 5796 |
| rs3093661 | SEQ ID NO: 1065 | SEQ ID NO: 3431 | SEQ ID NO: 5797 |
| rs2252856 | SEQ ID NO: 1066 | SEQ ID NO: 3432 | SEQ ID NO: 5798 |
| rs2857177 | SEQ ID NO: 1067 | SEQ ID NO: 3433 | SEQ ID NO: 5799 |
| rs9968 | SEQ ID NO: 1068 | SEQ ID NO: 3434 | SEQ ID NO: 5800 |
| rs365052 | SEQ ID NO: 1069 | SEQ ID NO: 3435 | SEQ ID NO: 5801 |
| rs1800624 | SEQ ID NO: 1070 | SEQ ID NO: 3436 | SEQ ID NO: 5802 |
| rs2856715 | SEQ ID NO: 1071 | SEQ ID NO: 3437 | SEQ ID NO: 5803 |
| rs3130444 | SEQ ID NO: 1072 | SEQ ID NO: 3438 | SEQ ID NO: 5804 |
| rs2071551 | SEQ ID NO: 1073 | SEQ ID NO: 3439 | SEQ ID NO: 5805 |
| rs2301221 | SEQ ID NO: 1074 | SEQ ID NO: 3440 | SEQ ID NO: 5806 |
| rs6927322 | SEQ ID NO: 1075 | SEQ ID NO: 3441 | SEQ ID NO: 5807 |
| rs7768179 | SEQ ID NO: 1076 | SEQ ID NO: 3442 | SEQ ID NO: 5808 |
| rs6913646 | SEQ ID NO: 1077 | SEQ ID NO: 3443 | SEQ ID NO: 5809 |
| rs2256820 | SEQ ID NO: 1078 | SEQ ID NO: 3444 | SEQ ID NO: 5810 |
| rs4713410 | SEQ ID NO: 1079 | SEQ ID NO: 3445 | SEQ ID NO: 5811 |
| rs872956 | SEQ ID NO: 1080 | SEQ ID NO: 3446 | SEQ ID NO: 5812 |
| rs4959077 | SEQ ID NO: 1081 | SEQ ID NO: 3447 | SEQ ID NO: 5813 |
| rs2071282 | SEQ ID NO: 1082 | SEQ ID NO: 3448 | SEQ ID NO: 5814 |
| rs1264328 | SEQ ID NO: 1083 | SEQ ID NO: 3449 | SEQ ID NO: 5815 |
| rs3918149 | SEQ ID NO: 1084 | SEQ ID NO: 3450 | SEQ ID NO: 5816 |
| rs2157051 | SEQ ID NO: 1085 | SEQ ID NO: 3451 | SEQ ID NO: 5817 |
| rs7750514 | SEQ ID NO: 1086 | SEQ ID NO: 3452 | SEQ ID NO: 5818 |
| rs3128964 | SEQ ID NO: 1087 | SEQ ID NO: 3453 | SEQ ID NO: 5819 |
| rs6912877 | SEQ ID NO: 1088 | SEQ ID NO: 3454 | SEQ ID NO: 5820 |
| rs1541268 | SEQ ID NO: 1089 | SEQ ID NO: 3455 | SEQ ID NO: 5821 |
| rs6906175 | SEQ ID NO: 1090 | SEQ ID NO: 3456 | SEQ ID NO: 5822 |
| rs2074506 | SEQ ID NO: 1091 | SEQ ID NO: 3457 | SEQ ID NO: 5823 |
| rs2239523 | SEQ ID NO: 1092 | SEQ ID NO: 3458 | SEQ ID NO: 5824 |
| rs2242665 | SEQ ID NO: 1093 | SEQ ID NO: 3459 | SEQ ID NO: 5825 |
| rs3131300 | SEQ ID NO: 1094 | SEQ ID NO: 3460 | SEQ ID NO: 5826 |
| rs465223 | SEQ ID NO: 1095 | SEQ ID NO: 3461 | SEQ ID NO: 5827 |
| rs3819299 | SEQ ID NO: 1096 | SEQ ID NO: 3462 | SEQ ID NO: 5828 |
| rs2844524 | SEQ ID NO: 1097 | SEQ ID NO: 3463 | SEQ ID NO: 5829 |
| rs6915440 | SEQ ID NO: 1098 | SEQ ID NO: 3464 | SEQ ID NO: 5830 |
| rs1614309 | SEQ ID NO: 1099 | SEQ ID NO: 3465 | SEQ ID NO: 5831 |
| rs2076183 | SEQ ID NO: 1100 | SEQ ID NO: 3466 | SEQ ID NO: 5832 |
| rs3763311 | SEQ ID NO: 1101 | SEQ ID NO: 3467 | SEQ ID NO: 5833 |
| rs3097650 | SEQ ID NO: 1102 | SEQ ID NO: 3468 | SEQ ID NO: 5834 |
| rs2743945 | SEQ ID NO: 1103 | SEQ ID NO: 3469 | SEQ ID NO: 5835 |
| rs1737087 | SEQ ID NO: 1104 | SEQ ID NO: 3470 | SEQ ID NO: 5836 |
| rs1611165 | SEQ ID NO: 1105 | SEQ ID NO: 3471 | SEQ ID NO: 5837 |
| rs3828913 | SEQ ID NO: 1106 | SEQ ID NO: 3472 | SEQ ID NO: 5838 |
| rs3763312 | SEQ ID NO: 1107 | SEQ ID NO: 3473 | SEQ ID NO: 5839 |
| rs2269711 | SEQ ID NO: 1108 | SEQ ID NO: 3474 | SEQ ID NO: 5840 |
| rs2243873 | SEQ ID NO: 1109 | SEQ ID NO: 3475 | SEQ ID NO: 5841 |
| rs2072108 | SEQ ID NO: 1110 | SEQ ID NO: 3476 | SEQ ID NO: 5842 |
| rs7770882 | SEQ ID NO: 1111 | SEQ ID NO: 3477 | SEQ ID NO: 5843 |
| rs1736935 | SEQ ID NO: 1112 | SEQ ID NO: 3478 | SEQ ID NO: 5844 |
| rs3823420 | SEQ ID NO: 1113 | SEQ ID NO: 3479 | SEQ ID NO: 5845 |
| rs2071571 | SEQ ID NO: 1114 | SEQ ID NO: 3480 | SEQ ID NO: 5846 |
| rs3093726 | SEQ ID NO: 1115 | SEQ ID NO: 3481 | SEQ ID NO: 5847 |
| rs973233 | SEQ ID NO: 1116 | SEQ ID NO: 3482 | SEQ ID NO: 5848 |
| rs1150772 | SEQ ID NO: 1117 | SEQ ID NO: 3483 | SEQ ID NO: 5849 |
| rs1264301 | SEQ ID NO: 1118 | SEQ ID NO: 3484 | SEQ ID NO: 5850 |
| rs367398 | SEQ ID NO: 1119 | SEQ ID NO: 3485 | SEQ ID NO: 5851 |
| rs241451 | SEQ ID NO: 1120 | SEQ ID NO: 3486 | SEQ ID NO: 5852 |
| rs2076528 | SEQ ID NO: 1121 | SEQ ID NO: 3487 | SEQ ID NO: 5853 |
| rs3115615 | SEQ ID NO: 1122 | SEQ ID NO: 3488 | SEQ ID NO: 5854 |
| rs2228088 | SEQ ID NO: 1123 | SEQ ID NO: 3489 | SEQ ID NO: 5855 |
| rs2269611 | SEQ ID NO: 1124 | SEQ ID NO: 3490 | SEQ ID NO: 5856 |
| rs408036 | SEQ ID NO: 1125 | SEQ ID NO: 3491 | SEQ ID NO: 5857 |
| rs3130998 | SEQ ID NO: 1126 | SEQ ID NO: 3492 | SEQ ID NO: 5858 |
| rs2022537 | SEQ ID NO: 1127 | SEQ ID NO: 3493 | SEQ ID NO: 5859 |
| rs2239520 | SEQ ID NO: 1128 | SEQ ID NO: 3494 | SEQ ID NO: 5860 |
| rs4993139 | SEQ ID NO: 1129 | SEQ ID NO: 3495 | SEQ ID NO: 5861 |
| rs3094215 | SEQ ID NO: 1130 | SEQ ID NO: 3496 | SEQ ID NO: 5862 |
| rs1063379 | SEQ ID NO: 1131 | SEQ ID NO: 3497 | SEQ ID NO: 5863 |
| rs259942 | SEQ ID NO: 1132 | SEQ ID NO: 3498 | SEQ ID NO: 5864 |
| rs3734861 | SEQ ID NO: 1133 | SEQ ID NO: 3499 | SEQ ID NO: 5865 |
| rs2499 | SEQ ID NO: 1134 | SEQ ID NO: 3500 | SEQ ID NO: 5866 |
| rs4713250 | SEQ ID NO: 1135 | SEQ ID NO: 3501 | SEQ ID NO: 5867 |
| rs2269426 | SEQ ID NO: 1136 | SEQ ID NO: 3502 | SEQ ID NO: 5868 |
| rs887467 | SEQ ID NO: 1137 | SEQ ID NO: 3503 | SEQ ID NO: 5869 |
| rs2269712 | SEQ ID NO: 1138 | SEQ ID NO: 3504 | SEQ ID NO: 5870 |
| rs2302396 | SEQ ID NO: 1139 | SEQ ID NO: 3505 | SEQ ID NO: 5871 |
| rs2008560 | SEQ ID NO: 1140 | SEQ ID NO: 3506 | SEQ ID NO: 5872 |
| rs1480383 | SEQ ID NO: 1141 | SEQ ID NO: 3507 | SEQ ID NO: 5873 |
| rs3129997 | SEQ ID NO: 1142 | SEQ ID NO: 3508 | SEQ ID NO: 5874 |
| rs2517588 | SEQ ID NO: 1143 | SEQ ID NO: 3509 | SEQ ID NO: 5875 |
| rs465474 | SEQ ID NO: 1144 | SEQ ID NO: 3510 | SEQ ID NO: 5876 |
| rs3218812 | SEQ ID NO: 1145 | SEQ ID NO: 3511 | SEQ ID NO: 5877 |
| rs2523791 | SEQ ID NO: 1146 | SEQ ID NO: 3512 | SEQ ID NO: 5878 |
| rs3828912 | SEQ ID NO: 1147 | SEQ ID NO: 3513 | SEQ ID NO: 5879 |
| rs2517444 | SEQ ID NO: 1148 | SEQ ID NO: 3514 | SEQ ID NO: 5880 |
| rs2257347 | SEQ ID NO: 1149 | SEQ ID NO: 3515 | SEQ ID NO: 5881 |
| rs364950 | SEQ ID NO: 1150 | SEQ ID NO: 3516 | SEQ ID NO: 5882 |
| rs2006164 | SEQ ID NO: 1151 | SEQ ID NO: 3517 | SEQ ID NO: 5883 |
| rs2857172 | SEQ ID NO: 1152 | SEQ ID NO: 3518 | SEQ ID NO: 5884 |
| rs3873243 | SEQ ID NO: 1153 | SEQ ID NO: 3519 | SEQ ID NO: 5885 |
| rs3763327 | SEQ ID NO: 1154 | SEQ ID NO: 3520 | SEQ ID NO: 5886 |
| rs1061801 | SEQ ID NO: 1155 | SEQ ID NO: 3521 | SEQ ID NO: 5887 |
| rs4713519 | SEQ ID NO: 1156 | SEQ ID NO: 3522 | SEQ ID NO: 5888 |
| rs909722 | SEQ ID NO: 1157 | SEQ ID NO: 3523 | SEQ ID NO: 5889 |
| rs2213569 | SEQ ID NO: 1158 | SEQ ID NO: 3524 | SEQ ID NO: 5890 |
| rs644827 | SEQ ID NO: 1159 | SEQ ID NO: 3525 | SEQ ID NO: 5891 |
| rs3763341 | SEQ ID NO: 1160 | SEQ ID NO: 3526 | SEQ ID NO: 5892 |
| rs2254287 | SEQ ID NO: 1161 | SEQ ID NO: 3527 | SEQ ID NO: 5893 |
| rs769178 | SEQ ID NO: 1162 | SEQ ID NO: 3528 | SEQ ID NO: 5894 |
| rs2270190 | SEQ ID NO: 1163 | SEQ ID NO: 3529 | SEQ ID NO: 5895 |
| rs986521 | SEQ ID NO: 1164 | SEQ ID NO: 3530 | SEQ ID NO: 5896 |
| rs206775 | SEQ ID NO: 1165 | SEQ ID NO: 3531 | SEQ ID NO: 5897 |
| rs241447 | SEQ ID NO: 1166 | SEQ ID NO: 3532 | SEQ ID NO: 5898 |
| rs2157336 | SEQ ID NO: 1167 | SEQ ID NO: 3533 | SEQ ID NO: 5899 |
| rs1044507 | SEQ ID NO: 1168 | SEQ ID NO: 3534 | SEQ ID NO: 5900 |
| rs3094204 | SEQ ID NO: 1169 | SEQ ID NO: 3535 | SEQ ID NO: 5901 |
| rs1611613 | SEQ ID NO: 1170 | SEQ ID NO: 3536 | SEQ ID NO: 5902 |
| rs1736927 | SEQ ID NO: 1171 | SEQ ID NO: 3537 | SEQ ID NO: 5903 |
| rs2855438 | SEQ ID NO: 1172 | SEQ ID NO: 3538 | SEQ ID NO: 5904 |
| rs3819284 | SEQ ID NO: 1173 | SEQ ID NO: 3539 | SEQ ID NO: 5905 |
| rs6913471 | SEQ ID NO: 1174 | SEQ ID NO: 3540 | SEQ ID NO: 5906 |
| rs969892 | SEQ ID NO: 1175 | SEQ ID NO: 3541 | SEQ ID NO: 5907 |
| rs2516507 | SEQ ID NO: 1176 | SEQ ID NO: 3542 | SEQ ID NO: 5908 |
| rs2071552 | SEQ ID NO: 1177 | SEQ ID NO: 3543 | SEQ ID NO: 5909 |
| rs375256 | SEQ ID NO: 1178 | SEQ ID NO: 3544 | SEQ ID NO: 5910 |
| rs2074465 | SEQ ID NO: 1179 | SEQ ID NO: 3545 | SEQ ID NO: 5911 |
| rs3130712 | SEQ ID NO: 1180 | SEQ ID NO: 3546 | SEQ ID NO: 5912 |
| rs2286654 | SEQ ID NO: 1181 | SEQ ID NO: 3547 | SEQ ID NO: 5913 |
| rs1265089 | SEQ ID NO: 1182 | SEQ ID NO: 3548 | SEQ ID NO: 5914 |
| rs915652 | SEQ ID NO: 1183 | SEQ ID NO: 3549 | SEQ ID NO: 5915 |
| rs362513 | SEQ ID NO: 1184 | SEQ ID NO: 3550 | SEQ ID NO: 5916 |
| rs3736710 | SEQ ID NO: 1185 | SEQ ID NO: 3551 | SEQ ID NO: 5917 |
| rs3130662 | SEQ ID NO: 1186 | SEQ ID NO: 3552 | SEQ ID NO: 5918 |
| rs2524056 | SEQ ID NO: 1187 | SEQ ID NO: 3553 | SEQ ID NO: 5919 |
| rs2395039 | SEQ ID NO: 1188 | SEQ ID NO: 3554 | SEQ ID NO: 5920 |
| rs683208 | SEQ ID NO: 1189 | SEQ ID NO: 3555 | SEQ ID NO: 5921 |
| rs1264320 | SEQ ID NO: 1190 | SEQ ID NO: 3556 | SEQ ID NO: 5922 |
| rs2074492 | SEQ ID NO: 1191 | SEQ ID NO: 3557 | SEQ ID NO: 5923 |
| rs4524656 | SEQ ID NO: 1192 | SEQ ID NO: 3558 | SEQ ID NO: 5924 |
| rs3130934 | SEQ ID NO: 1193 | SEQ ID NO: 3559 | SEQ ID NO: 5925 |
| rs1264457 | SEQ ID NO: 1194 | SEQ ID NO: 3560 | SEQ ID NO: 5926 |
| rs2242667 | SEQ ID NO: 1195 | SEQ ID NO: 3561 | SEQ ID NO: 5927 |
| rs7739536 | SEQ ID NO: 1196 | SEQ ID NO: 3562 | SEQ ID NO: 5928 |
| rs409715 | SEQ ID NO: 1197 | SEQ ID NO: 3563 | SEQ ID NO: 5929 |
| rs3828914 | SEQ ID NO: 1198 | SEQ ID NO: 3564 | SEQ ID NO: 5930 |
| rs2272991 | SEQ ID NO: 1199 | SEQ ID NO: 3565 | SEQ ID NO: 5931 |
| rs1362061 | SEQ ID NO: 1200 | SEQ ID NO: 3566 | SEQ ID NO: 5932 |
| rs3132377 | SEQ ID NO: 1201 | SEQ ID NO: 3567 | SEQ ID NO: 5933 |
| rs743771 | SEQ ID NO: 1202 | SEQ ID NO: 3568 | SEQ ID NO: 5934 |
| rs2523612 | SEQ ID NO: 1203 | SEQ ID NO: 3569 | SEQ ID NO: 5935 |
| rs4713220 | SEQ ID NO: 1204 | SEQ ID NO: 3570 | SEQ ID NO: 5936 |
| rs2301224 | SEQ ID NO: 1205 | SEQ ID NO: 3571 | SEQ ID NO: 5937 |
| rs3106191 | SEQ ID NO: 1206 | SEQ ID NO: 3572 | SEQ ID NO: 5938 |
| rs3749982 | SEQ ID NO: 1207 | SEQ ID NO: 3573 | SEQ ID NO: 5939 |
| rs1610678 | SEQ ID NO: 1208 | SEQ ID NO: 3574 | SEQ ID NO: S940 |
| rs2524055 | SEQ ID NO: 1209 | SEQ ID NO: 3575 | SEQ ID NO: 5941 |
| rs2073721 | SEQ ID NO: 1210 | SEQ ID NO: 3576 | SEQ ID NO: 5942 |
| rs3132496 | SEQ ID NO: 1211 | SEQ ID NO: 3577 | SEQ ID NO: 5943 |
| rs2071653 | SEQ ID NO: 1212 | SEQ ID NO: 3578 | SEQ ID NO: 5944 |
| rs7772982 | SEQ ID NO: 1213 | SEQ ID NO: 3579 | SEQ ID NO: 5945 |
| rs2524094 | SEQ ID NO: 1214 | SEQ ID NO: 3580 | SEQ ID NO: 5946 |
| rs2252863 | SEQ ID NO: 1215 | SEQ ID NO: 3581 | SEQ ID NO: 5947 |
| rs2532939 | SEQ ID NO: 1216 | SEQ ID NO: 3582 | SEQ ID NO: 5948 |
| rs3129793 | SEQ ID NO: 1217 | SEQ ID NO: 3583 | SEQ ID NO: 5949 |
| rs3130926 | SEQ ID NO: 1218 | SEQ ID NO: 3584 | SEQ ID NO: 5950 |
| rs707918 | SEQ ID NO: 1219 | SEQ ID NO: 3585 | SEQ ID NO: 5951 |
| rs6925178 | SEQ ID NO: 1220 | SEQ ID NO: 3586 | SEQ ID NO: 5952 |
| rs6456902 | SEQ ID NO: 1221 | SEQ ID NO: 3587 | SEQ ID NO: 5953 |
| rs1610682 | SEQ ID NO: 1222 | SEQ ID NO: 3588 | SEQ ID NO: 5954 |
| rs2073720 | SEQ ID NO: 1223 | SEQ ID NO: 3589 | SEQ ID NO: 5955 |
| rs2071785 | SEQ ID NO: 1224 | SEQ ID NO: 3590 | SEQ ID NO: 5956 |
| rs2856993 | SEQ ID NO: 1225 | SEQ ID NO: 3591 | SEQ ID NO: 5957 |
| rs4959012 | SEQ ID NO: 1226 | SEQ ID NO: 3592 | SEQ ID NO: 5958 |
| rs614549 | SEQ ID NO: 1227 | SEQ ID NO: 3593 | SEQ ID NO: 5959 |
| rs3749963 | SEQ ID NO: 1228 | SEQ ID NO: 3594 | SEQ ID NO: 5960 |
| rs3757324 | SEQ ID NO: 1229 | SEQ ID NO: 3595 | SEQ ID NO: 5961 |
| rs6457150 | SEQ ID NO: 1230 | SEQ ID NO: 3596 | SEQ ID NO: 5962 |
| rs915651 | SEQ ID NO: 1231 | SEQ ID NO: 3597 | SEQ ID NO: 5963 |
| rs3130456 | SEQ ID NO: 1232 | SEQ ID NO: 3598 | SEQ ID NO: 5964 |
| rs1150739 | SEQ ID NO: 1233 | SEQ ID NO: 3599 | SEQ ID NO: 5965 |
| rs1611617 | SEQ ID NO: 1234 | SEQ ID NO: 3600 | SEQ ID NO: 5966 |
| rs3094139 | SEQ ID NO: 1235 | SEQ ID NO: 3601 | SEQ ID NO: 5967 |
| rs2394414 | SEQ ID NO: 1236 | SEQ ID NO: 3602 | SEQ ID NO: 5968 |
| rs11724 | SEQ ID NO: 1237 | SEQ ID NO: 3603 | SEQ ID NO: 5969 |
| rs1611185 | SEQ ID NO: 1238 | SEQ ID NO: 3604 | SEQ ID NO: 5970 |
| rs6940440 | SEQ ID NO: 1239 | SEQ ID NO: 3605 | SEQ ID NO: 5971 |
| rs7452208 | SEQ ID NO: 1240 | SEQ ID NO: 3606 | SEQ ID NO: 5972 |
| rs3763342 | SEQ ID NO: 1241 | SEQ ID NO: 3607 | SEQ ID NO: 5973 |
| rs2518030 | SEQ ID NO: 1242 | SEQ ID NO: 3608 | SEQ ID NO: 5974 |
| rs7772174 | SEQ ID NO: 1243 | SEQ ID NO: 3609 | SEQ ID NO: 5975 |
| rs3869097 | SEQ ID NO: 1244 | SEQ ID NO: 3610 | SEQ ID NO: 5976 |
| rs2272593 | SEQ ID NO: 1245 | SEQ ID NO: 3611 | SEQ ID NO: 5977 |
| rs3129792 | SEQ ID NO: 1246 | SEQ ID NO: 3612 | SEQ ID NO: 5978 |
| rs1264305 | SEQ ID NO: 1247 | SEQ ID NO: 3613 | SEQ ID NO: 5979 |
| rs4959047 | SEQ ID NO: 1248 | SEQ ID NO: 3614 | SEQ ID NO: 5980 |
| rs4713436 | SEQ ID NO: 1249 | SEQ ID NO: 3615 | SEQ ID NO: 5981 |
| rs7829 | SEQ ID NO: 1250 | SEQ ID NO: 3616 | SEQ ID NO: 5982 |
| rs3094696 | SEQ ID NO: 1251 | SEQ ID NO: 3617 | SEQ ID NO: 5983 |
| rs3828881 | SEQ ID NO: 1252 | SEQ ID NO: 3618 | SEQ ID NO: 5984 |
| rs3094000 | SEQ ID NO: 1253 | SEQ ID NO: 3619 | SEQ ID NO: 5985 |
| rs2855440 | SEQ ID NO: 1254 | SEQ ID NO: 3620 | SEQ ID NO: 5986 |
| rs6457697 | SEQ ID NO: 1255 | SEQ ID NO: 3621 | SEQ ID NO: 5987 |
| rs6919044 | SEQ ID NO: 1256 | SEQ ID NO: 3622 | SEQ ID NO: 5988 |
| rs1013518 | SEQ ID NO: 1257 | SEQ ID NO: 3623 | SEQ ID NO: 5989 |
| rs2144426 | SEQ ID NO: 1258 | SEQ ID NO: 3624 | SEQ ID NO: 5990 |
| rs4267952 | SEQ ID NO: 1259 | SEQ ID NO: 3625 | SEQ ID NO: 5991 |
| rs4947339 | SEQ ID NO: 1260 | SEQ ID NO: 3626 | SEQ ID NO: 5992 |
| rs4618569 | SEQ ID NO: 1261 | SEQ ID NO: 3627 | SEQ ID NO: 5993 |
| rs7767288 | SEQ ID NO: 1262 | SEQ ID NO: 3628 | SEQ ID NO: 5994 |
| rs3752528 | SEQ ID NO: 1263 | SEQ ID NO: 3629 | SEQ ID NO: 5995 |
| rs3956986 | SEQ ID NO: 1264 | SEQ ID NO: 3630 | SEQ ID NO: 5996 |
| rs2285320 | SEQ ID NO: 1265 | SEQ ID NO: 3631 | SEQ ID NO: 5997 |
| rs6938690 | SEQ ID NO: 1266 | SEQ ID NO: 3632 | SEQ ID NO: 5998 |
| rs2532932 | SEQ ID NO: 1267 | SEQ ID NO: 3633 | SEQ ID NO: 5999 |
| rs1634725 | SEQ ID NO: 1268 | SEQ ID NO: 3634 | SEQ ID NO: 6000 |
| rs3117097 | SEQ ID NO: 1269 | SEQ ID NO: 3635 | SEQ ID NO: 6001 |
| rs2621421 | SEQ ID NO: 1270 | SEQ ID NO: 3636 | SEQ ID NO: 6002 |
| rs2076541 | SEQ ID NO: 1271 | SEQ ID NO: 3637 | SEQ ID NO: 6003 |
| rs3094613 | SEQ ID NO: 1272 | SEQ ID NO: 3638 | SEQ ID NO: 6004 |
| rs4711186 | SEQ ID NO: 1273 | SEQ ID NO: 3639 | SEQ ID NO: 6005 |
| rs3130165 | SEQ ID NO: 1274 | SEQ ID NO: 3640 | SEQ ID NO: 6006 |
| rs2395312 | SEQ ID NO: 1275 | SEQ ID NO: 3641 | SEQ ID NO: 6007 |
| rs1627764 | SEQ ID NO: 1276 | SEQ ID NO: 3642 | SEQ ID NO: 6008 |
| rs204896 | SEQ ID NO: 1277 | SEQ ID NO: 3643 | SEQ ID NO: 6009 |
| rs7750714 | SEQ ID NO: 1278 | SEQ ID NO: 3644 | SEQ ID NO: 6010 |
| rs1054684 | SEQ ID NO: 1279 | SEQ ID NO: 3645 | SEQ ID NO: 6011 |
| rs7768931 | SEQ ID NO: 1280 | SEQ ID NO: 3646 | SEQ ID NO: 6012 |
| rs2213573 | SEQ ID NO: 1281 | SEQ ID NO: 3647 | SEQ ID NO: 6013 |
| rs241410 | SEQ ID NO: 1282 | SEQ ID NO: 3648 | SEQ ID NO: 6014 |
| rs2523734 | SEQ ID NO: 1283 | SEQ ID NO: 3649 | SEQ ID NO: 6015 |
| rs2163472 | SEQ ID NO: 1284 | SEQ ID NO: 3650 | SEQ ID NO: 6016 |
| rs6930003 | SEQ ID NO: 1285 | SEQ ID NO: 3651 | SEQ ID NO: 6017 |
| rs4713193 | SEQ ID NO: 1286 | SEQ ID NO: 3652 | SEQ ID NO: 6018 |
| rs1264321 | SEQ ID NO: 1287 | SEQ ID NO: 3653 | SEQ ID NO: 6019 |
| rs204878 | SEQ ID NO: 1288 | SEQ ID NO: 3654 | SEQ ID NO: 6020 |
| rs2301752 | SEQ ID NO: 1289 | SEQ ID NO: 3655 | SEQ ID NO: 6021 |
| rs6901830 | SEQ ID NO: 1290 | SEQ ID NO: 3656 | SEQ ID NO: 6022 |
| rs3998159 | SEQ ID NO: 1291 | SEQ ID NO: 3657 | SEQ ID NO: 6023 |
| rs2246986 | SEQ ID NO: 1292 | SEQ ID NO: 3658 | SEQ ID NO: 6024 |
| rs3117113 | SEQ ID NO: 1293 | SEQ ID NO: 3659 | SEQ ID NO: 6025 |
| rs2857128 | SEQ ID NO: 1294 | SEQ ID NO: 3660 | SEQ ID NO: 6026 |
| rs2516643 | SEQ ID NO: 1295 | SEQ ID NO: 3661 | SEQ ID NO: 6027 |
| rs3117103 | SEQ ID NO: 1296 | SEQ ID NO: 3662 | SEQ ID NO: 6028 |
| rs2233965 | SEQ ID NO: 1297 | SEQ ID NO: 3663 | SEQ ID NO: 6029 |
| rs6916394 | SEQ ID NO: 1298 | SEQ ID NO: 3664 | SEQ ID NO: 6030 |
| rs2074485 | SEQ ID NO: 1299 | SEQ ID NO: 3665 | SEQ ID NO: 6031 |
| rs1619893 | SEQ ID NO: 1300 | SEQ ID NO: 3666 | SEQ ID NO: 6032 |
| rs4449615 | SEQ ID NO: 1301 | SEQ ID NO: 3667 | SEQ ID NO: 6033 |
| rs3115611 | SEQ ID NO: 1302 | SEQ ID NO: 3668 | SEQ ID NO: 6034 |
| rs2301748 | SEQ ID NO: 1303 | SEQ ID NO: 3669 | SEQ ID NO: 6035 |
| rs3129845 | SEQ ID NO: 1304 | SEQ ID NO: 3670 | SEQ ID NO: 6036 |
| rs3130829 | SEQ ID NO: 1305 | SEQ ID NO: 3671 | SEQ ID NO: 6037 |
| rs6457125 | SEQ ID NO: 1306 | SEQ ID NO: 3672 | SEQ ID NO: 6038 |
| rs2571402 | SEQ ID NO: 1307 | SEQ ID NO: 3673 | SEQ ID NO: 6039 |
| rs2157605 | SEQ ID NO: 1308 | SEQ ID NO: 3674 | SEQ ID NO: 6040 |
| rs2275853 | SEQ ID NO: 1309 | SEQ ID NO: 3675 | SEQ ID NO: 6041 |
| rs3132710 | SEQ ID NO: 1310 | SEQ ID NO: 3676 | SEQ ID NO: 6042 |
| rs2854005 | SEQ ID NO: 1311 | SEQ ID NO: 3677 | SEQ ID NO: 6043 |
| rs1011985 | SEQ ID NO: 1312 | SEQ ID NO: 3678 | SEQ ID NO: 6044 |
| rs2075789 | SEQ ID NO: 1313 | SEQ ID NO: 3679 | SEQ ID NO: 6045 |
| rs2076182 | SEQ ID NO: 1314 | SEQ ID NO: 3680 | SEQ ID NO: 6046 |
| rs3132684 | SEQ ID NO: 1315 | SEQ ID NO: 3681 | SEQ ID NO: 6047 |
| rs238877 | SEQ ID NO: 1316 | SEQ ID NO: 3682 | SEQ ID NO: 6048 |
| rs2072681 | SEQ ID NO: 1317 | SEQ ID NO: 3683 | SEQ ID NO: 6049 |
| rs6919908 | SEQ ID NO: 1318 | SEQ ID NO: 3684 | SEQ ID NO: 6050 |
| rs2647045 | SEQ ID NO: 1319 | SEQ ID NO: 3685 | SEQ ID NO: 6051 |
| rs1632986 | SEQ ID NO: 1320 | SEQ ID NO: 3686 | SEQ ID NO: 6052 |
| rs2395470 | SEQ ID NO: 1321 | SEQ ID NO: 3687 | SEQ ID NO: 6053 |
| rs915668 | SEQ ID NO: 1322 | SEQ ID NO: 3688 | SEQ ID NO: 6054 |
| rs2295119 | SEQ ID NO: 1323 | SEQ ID NO: 3689 | SEQ ID NO: 6055 |
| rs2071536 | SEQ ID NO: 1324 | SEQ ID NO: 3690 | SEQ ID NO: 6056 |
| rs3130827 | SEQ ID NO: 1325 | SEQ ID NO: 3691 | SEQ ID NO: 6057 |
| rs3093556 | SEQ ID NO: 1326 | SEQ ID NO: 3692 | SEQ ID NO: 6058 |
| rs3734818 | SEQ ID NO: 1327 | SEQ ID NO: 3693 | SEQ ID NO: 6059 |
| rs4713270 | SEQ ID NO: 1328 | SEQ ID NO: 3694 | SEQ ID NO: 6060 |
| rs1988565 | SEQ ID NO: 1329 | SEQ ID NO: 3695 | SEQ ID NO: 6061 |
| rs1233333 | SEQ ID NO: 1330 | SEQ ID NO: 3696 | SEQ ID NO: 6062 |
| rs2074489 | SEQ ID NO: 1331 | SEQ ID NO: 3697 | SEQ ID NO: 6063 |
| rs1736981 | SEQ ID NO: 1332 | SEQ ID NO: 3698 | SEQ ID NO: 6064 |
| rs3129005 | SEQ ID NO: 1333 | SEQ ID NO: 3699 | SEQ ID NO: 6065 |
| rs3998357 | SEQ ID NO: 1334 | SEQ ID NO: 3700 | SEQ ID NO: 6066 |
| rs3132645 | SEQ ID NO: 1335 | SEQ ID NO: 3701 | SEQ ID NO: 6067 |
| rs1264548 | SEQ ID NO: 1336 | SEQ ID NO: 3702 | SEQ ID NO: 6068 |
| rs2428495 | SEQ ID NO: 1337 | SEQ ID NO: 3703 | SEQ ID NO: 6069 |
| rs3817824 | SEQ ID NO: 1338 | SEQ ID NO: 3704 | SEQ ID NO: 6070 |
| rs1632948 | SEQ ID NO: 1339 | SEQ ID NO: 3705 | SEQ ID NO: 6071 |
| rs1736998 | SEQ ID NO: 1340 | SEQ ID NO: 3706 | SEQ ID NO: 6072 |
| rs2535252 | SEQ ID NO: 1341 | SEQ ID NO: 3707 | SEQ ID NO: 6073 |
| rs1625485 | SEQ ID NO: 1342 | SEQ ID NO: 3708 | SEQ ID NO: 6074 |
| rs1811197 | SEQ ID NO: 1343 | SEQ ID NO: 3709 | SEQ ID NO: 6075 |
| rs2524173 | SEQ ID NO: 1344 | SEQ ID NO: 3710 | SEQ ID NO: 6076 |
| rs6925580 | SEQ ID NO: 1345 | SEQ ID NO: 3711 | SEQ ID NO: 6077 |
| rs3828903 | SEQ ID NO: 1346 | SEQ ID NO: 3712 | SEQ ID NO: 6078 |
| rs2596450 | SEQ ID NO: 1347 | SEQ ID NO: 3713 | SEQ ID NO: 6079 |
| rs6457042 | SEQ ID NO: 1348 | SEQ ID NO: 3714 | SEQ ID NO: 6080 |
| rs1799908 | SEQ ID NO: 1349 | SEQ ID NO: 3715 | SEQ ID NO: 6081 |
| rs8365 | SEQ ID NO: 1350 | SEQ ID NO: 3716 | SEQ ID NO: 6082 |
| rs3823340 | SEQ ID NO: 1351 | SEQ ID NO: 3717 | SEQ ID NO: 6083 |
| rs1611589 | SEQ ID NO: 1352 | SEQ ID NO: 3718 | SEQ ID NO: 6084 |
| rs2071291 | SEQ ID NO: 1353 | SEQ ID NO: 3719 | SEQ ID NO: 6085 |
| rs1233387 | SEQ ID NO: 1354 | SEQ ID NO: 3720 | SEQ ID NO: 6086 |
| rs6920823 | SEQ ID NO: 1355 | SEQ ID NO: 3721 | SEQ ID NO: 6087 |
| rs2251731 | SEQ ID NO: 1356 | SEQ ID NO: 3722 | SEQ ID NO: 6088 |
| rs6920830 | SEQ ID NO: 1357 | SEQ ID NO: 3723 | SEQ ID NO: 6089 |
| rs3873375 | SEQ ID NO: 1358 | SEQ ID NO: 3724 | SEQ ID NO: 6090 |
| rs1737004 | SEQ ID NO: 1359 | SEQ ID NO: 3725 | SEQ ID NO: 6091 |
| rs6457611 | SEQ ID NO: 1360 | SEQ ID NO: 3726 | SEQ ID NO: 6092 |
| rs2524088 | SEQ ID NO: 1361 | SEQ ID NO: 3727 | SEQ ID NO: 6093 |
| rs3749962 | SEQ ID NO: 1362 | SEQ ID NO: 3728 | SEQ ID NO: 6094 |
| rs1737085 | SEQ ID NO: 1363 | SEQ ID NO: 3729 | SEQ ID NO: 6095 |
| rs7382817 | SEQ ID NO: 1364 | SEQ ID NO: 3730 | SEQ ID NO: 6096 |
| rs2394413 | SEQ ID NO: 1365 | SEQ ID NO: 3731 | SEQ ID NO: 6097 |
| rs2073151 | SEQ ID NO: 1366 | SEQ ID NO: 3732 | SEQ ID NO: 6098 |
| rs1058026 | SEQ ID NO: 1367 | SEQ ID NO: 3733 | SEQ ID NO: 6099 |
| rs2272873 | SEQ ID NO: 1368 | SEQ ID NO: 3734 | SEQ ID NO: 6100 |
| rs1632933 | SEQ ID NO: 1369 | SEQ ID NO: 3735 | SEQ ID NO: 6101 |
| rs1632939 | SEQ ID NO: 1370 | SEQ ID NO: 3736 | SEQ ID NO: 6102 |
| rs3132704 | SEQ ID NO: 1371 | SEQ ID NO: 3737 | SEQ ID NO: 6103 |
| rs2523611 | SEQ ID NO: 1372 | SEQ ID NO: 3738 | SEQ ID NO: 6104 |
| rs3823313 | SEQ ID NO: 1373 | SEQ ID NO: 3739 | SEQ ID NO: 6105 |
| rs2524083 | SEQ ID NO: 1374 | SEQ ID NO: 3740 | SEQ ID NO: 6106 |
| rs983561 | SEQ ID NO: 1375 | SEQ ID NO: 3741 | SEQ ID NO: 6107 |
| rs915660 | SEQ ID NO: 1376 | SEQ ID NO: 3742 | SEQ ID NO: 6108 |
| rs1269839 | SEQ ID NO: 1377 | SEQ ID NO: 3743 | SEQ ID NO: 6109 |
| rs2050190 | SEQ ID NO: 1378 | SEQ ID NO: 3744 | SEQ ID NO: 6110 |
| rs8408 | SEQ ID NO: 1379 | SEQ ID NO: 3745 | SEQ ID NO: 6111 |
| rs3115573 | SEQ ID NO: 1380 | SEQ ID NO: 3746 | SEQ ID NO: 6112 |
| rs2230365 | SEQ ID NO: 1381 | SEQ ID NO: 3747 | SEQ ID NO: 6113 |
| rs2284178 | SEQ ID NO: 1382 | SEQ ID NO: 3748 | SEQ ID NO: 6114 |
| rs720467 | SEQ ID NO: 1383 | SEQ ID NO: 3749 | SEQ ID NO: 6115 |
| rs1842399 | SEQ ID NO: 1384 | SEQ ID NO: 3750 | SEQ ID NO: 6116 |
| rs2857106 | SEQ ID NO: 1385 | SEQ ID NO: 3751 | SEQ ID NO: 6117 |
| rs1894552 | SEQ ID NO: 1386 | SEQ ID NO: 3752 | SEQ ID NO: 6118 |
| rs1048664 | SEQ ID NO: 1387 | SEQ ID NO: 3753 | SEQ ID NO: 6119 |
| rs2076529 | SEQ ID NO: 1388 | SEQ ID NO: 3754 | SEQ ID NO: 6120 |
| rs2269422 | SEQ ID NO: 1389 | SEQ ID NO: 3755 | SEQ ID NO: 6121 |
| rs3135338 | SEQ ID NO: 1390 | SEQ ID NO: 3756 | SEQ ID NO: 6122 |
| rs3129907 | SEQ ID NO: 1391 | SEQ ID NO: 3757 | SEQ ID NO: 6123 |
| rs2621413 | SEQ ID NO: 1392 | SEQ ID NO: 3758 | SEQ ID NO: 6124 |
| rs3129848 | SEQ ID NO: 1393 | SEQ ID NO: 3759 | SEQ ID NO: 6125 |
| rs2395253 | SEQ ID NO: 1394 | SEQ ID NO: 3760 | SEQ ID NO: 6126 |
| rs3135365 | SEQ ID NO: 1395 | SEQ ID NO: 3761 | SEQ ID NO: 6127 |
| rs2395106 | SEQ ID NO: 1396 | SEQ ID NO: 3762 | SEQ ID NO: 6128 |
| rs2285796 | SEQ ID NO: 1397 | SEQ ID NO: 3763 | SEQ ID NO: 6129 |
| rs2284177 | SEQ ID NO: 1398 | SEQ ID NO: 3764 | SEQ ID NO: 6130 |
| rs3115563 | SEQ ID NO: 1399 | SEQ ID NO: 3765 | SEQ ID NO: 6131 |
| rs2074477 | SEQ ID NO: 1400 | SEQ ID NO: 3766 | SEQ ID NO: 6132 |
| rs2301755 | SEQ ID NO: 1401 | SEQ ID NO: 3767 | SEQ ID NO: 6133 |
| rs2057727 | SEQ ID NO: 1402 | SEQ ID NO: 3768 | SEQ ID NO: 6134 |
| rs2269705 | SEQ ID NO: 1403 | SEQ ID NO: 3769 | SEQ ID NO: 6135 |
| rs2076177 | SEQ ID NO: 1404 | SEQ ID NO: 3770 | SEQ ID NO: 6136 |
| rs2071285 | SEQ ID NO: 1405 | SEQ ID NO: 3771 | SEQ ID NO: 6137 |
| rs1109146 | SEQ ID NO: 1406 | SEQ ID NO: 3772 | SEQ ID NO: 6138 |
| rs2395178 | SEQ ID NO: 1407 | SEQ ID NO: 3773 | SEQ ID NO: 6139 |
| rs1978029 | SEQ ID NO: 1408 | SEQ ID NO: 3774 | SEQ ID NO: 6140 |
| rs408359 | SEQ ID NO: 1409 | SEQ ID NO: 3775 | SEQ ID NO: 6141 |
| rs2239529 | SEQ ID NO: 1410 | SEQ ID NO: 3776 | SEQ ID NO: 6142 |
| rs2269425 | SEQ ID NO: 1411 | SEQ ID NO: 3777 | SEQ ID NO: 6143 |
| rs2073048 | SEQ ID NO: 1412 | SEQ ID NO: 3778 | SEQ ID NO: 6144 |
| rs3806155 | SEQ ID NO: 1413 | SEQ ID NO: 3779 | SEQ ID NO: 6145 |
| rs436845 | SEQ ID NO: 1414 | SEQ ID NO: 3780 | SEQ ID NO: 6146 |
| rs434841 | SEQ ID NO: 1415 | SEQ ID NO: 3781 | SEQ ID NO: 6147 |
| rs1555115 | SEQ ID NO: 1416 | SEQ ID NO: 3782 | SEQ ID NO: 6148 |
| rs2284174 | SEQ ID NO: 1417 | SEQ ID NO:3783 | SEQ ID NO: 6149 |
| rs2239806 | SEQ ID NO: 1418 | SEQ ID NO: 3784 | SEQ ID NO: 6150 |
| rs1610714 | SEQ ID NO: 1419 | SEQ ID NO: 3785 | SEQ ID NO: 6151 |
| rs2071289 | SEQ ID NO: 1420 | SEQ ID NO: 3786 | SEQ ID NO: 6152 |
| rs1264302 | SEQ ID NO: 1421 | SEQ ID NO: 3787 | SEQ ID NO: 6153 |
| rs1265099 | SEQ ID NO: 1422 | SEQ ID NO: 3788 | SEQ ID NO: 6154 |
| rs926070 | SEQ ID NO: 1423 | SEQ ID NO: 3789 | SEQ ID NO: 6155 |
| rs1150738 | SEQ ID NO: 1424 | SEQ ID NO: 3790 | SEQ ID NO: 6156 |
| rs2075015 | SEQ ID NO: 1425 | SEQ ID NO: 3791 | SEQ ID NO: 6157 |
| rs1576 | SEQ ID NO: 1426 | SEQ ID NO: 3792 | SEQ ID NO: 6158 |
| rs2074482 | SEQ ID NO: 1427 | SEQ ID NO: 3793 | SEQ ID NO: 6159 |
| rs2076523 | SEQ ID NO: 1428 | SEQ ID NO: 3794 | SEQ ID NO: 6160 |
| rs2395182 | SEQ ID NO: 1429 | SEQ ID NO: 3795 | SEQ ID NO: 6161 |
| rs241429 | SEQ ID NO: 1430 | SEQ ID NO: 3796 | SEQ ID NO: 6162 |
| rs565448 | SEQ ID NO: 1431 | SEQ ID NO: 3797 | SEQ ID NO: 6163 |
| rs1383265 | SEQ ID NO: 1432 | SEQ ID NO: 3798 | SEQ ID NO: 6164 |
| rs1150771 | SEQ ID NO: 1433 | SEQ ID NO: 3799 | SEQ ID NO: 6165 |
| rs2284189 | SEQ ID NO: 1434 | SEQ ID NO: 3800 | SEQ ID NO: 6166 |
| rs3213484 | SEQ ID NO: 1435 | SEQ ID NO: 3801 | SEQ ID NO: 6167 |
| rs2856716 | SEQ ID NO: 1436 | SEQ ID NO: 3802 | SEQ ID NO: 6168 |
| rs2076526 | SEQ ID NO: 1437 | SEQ ID NO: 3803 | SEQ ID NO: 6169 |
| rs3830122 | SEQ ID NO: 1438 | SEQ ID NO: 3804 | SEQ ID NO: 6170 |
| rs1882 | SEQ ID NO: 1439 | SEQ ID NO: 3805 | SEQ ID NO: 6171 |
| rs512559 | SEQ ID NO: 1440 | SEQ ID NO: 3806 | SEQ ID NO: 6172 |
| rs2857102 | SEQ ID NO: 1441 | SEQ ID NO: 3807 | SEQ ID NO: 6173 |
| rs2856698 | SEQ ID NO: 1442 | SEQ ID NO: 3808 | SEQ ID NO: 6174 |
| rs204989 | SEQ ID NO: 1443 | SEQ ID NO: 3809 | SEQ ID NO: 6175 |
| rs419788 | SEQ ID NO: 1444 | SEQ ID NO: 3810 | SEQ ID NO: 6176 |
| rs986475 | SEQ ID NO: 1445 | SEQ ID NO: 3811 | SEQ ID NO: 6177 |
| rs2763979 | SEQ ID NO: 1446 | SEQ ID NO: 3812 | SEQ ID NO: 6178 |
| rs1062470 | SEQ ID NO: 1447 | SEQ ID NO: 3813 | SEQ ID NO: 6179 |
| rs2523668 | SEQ ID NO: 1448 | SEQ ID NO: 3814 | SEQ ID NO: 6180 |
| rs2524069 | SEQ ID NO: 1449 | SEQ ID NO: 3815 | SEQ ID NO: 6181 |
| rs3135339 | SEQ ID NO: 1450 | SEQ ID NO: 3816 | SEQ ID NO: 6182 |
| rs206776 | SEQ ID NO: 1451 | SEQ ID NO: 3817 | SEQ ID NO: 6183 |
| rs1237485 | SEQ ID NO: 1452 | SEQ ID NO: 3818 | SEQ ID NO: 6184 |
| rs805290 | SEQ ID NO: 1453 | SEQ ID NO: 3819 | SEQ ID NO: 6185 |
| rs805268 | SEQ ID NO: 1454 | SEQ ID NO: 3820 | SEQ ID NO: 6186 |
| rs1345228 | SEQ ID NO: 1455 | SEQ ID NO: 3821 | SEQ ID NO: 6187 |
| rs7742523 | SEQ ID NO: 1456 | SEQ ID NO: 3822 | SEQ ID NO: 6188 |
| rs1053924 | SEQ ID NO: 1457 | SEQ ID NO: 3823 | SEQ ID NO: 6189 |
| rs1270942 | SEQ ID NO: 1458 | SEQ ID NO: 3824 | SEQ ID NO: 6190 |
| rs3129872 | SEQ ID NO: 1459 | SEQ ID NO: 3825 | SEQ ID NO: 6191 |
| rs805256 | SEQ ID NO: 1460 | SEQ ID NO: 3826 | SEQ ID NO: 6192 |
| rs3116998 | SEQ ID NO: 1461 | SEQ ID NO: 3827 | SEQ ID NO: 6193 |
| rs2517484 | SEQ ID NO: 1462 | SEQ ID NO: 3828 | SEQ ID NO: 6194 |
| rs886398 | SEQ ID NO: 1463 | SEQ ID NO: 3829 | SEQ ID NO: 6195 |
| rs984778 | SEQ ID NO: 1464 | SEQ ID NO: 3830 | SEQ ID NO: 6196 |
| rs1819784 | SEQ ID NO: 1465 | SEQ ID NO: 3831 | SEQ ID NO: 6197 |
| rs537757 | SEQ ID NO: 1466 | SEQ ID NO: 3832 | SEQ ID NO: 6198 |
| rs213199 | SEQ ID NO: 1467 | SEQ ID NO: 3833 | SEQ ID NO: 6199 |
| rs7762289 | SEQ ID NO: 1468 | SEQ ID NO: 3834 | SEQ ID NO: 6200 |
| rs1264709 | SEQ ID NO: 1469 | SEQ ID NO: 3835 | SEQ ID NO: 6201 |
| rs2071352 | SEQ ID NO: 1470 | SEQ ID NO: 3836 | SEQ ID NO: 6202 |
| rs2213584 | SEQ ID NO: 1471 | SEQ ID NO: 3837 | SEQ ID NO: 6203 |
| rs7382138 | SEQ ID NO: 1472 | SEQ ID NO: 3838 | SEQ ID NO: 6204 |
| rs660550 | SEQ ID NO: 1473 | SEQ ID NO: 3839 | SEQ ID NO: 6205 |
| rs3094626 | SEQ ID NO: 1474 | SEQ ID NO: 3840 | SEQ ID NO: 6206 |
| rs130078 | SEQ ID NO: 1475 | SEQ ID NO: 3841 | SEQ ID NO: 6207 |
| rs1610647 | SEQ ID NO: 1476 | SEQ ID NO: 3842 | SEQ ID NO: 6208 |
| rs2071594 | SEQ ID NO: 1477 | SEQ ID NO: 3843 | SEQ ID NO: 6209 |
| rs393603 | SEQ ID NO: 1478 | SEQ ID NO: 3844 | SEQ ID NO: 6210 |
| rs1034325 | SEQ ID NO: 1479 | SEQ ID NO: 3845 | SEQ ID NO: 6211 |
| rs971570 | SEQ ID NO: 1480 | SEQ ID NO: 3846 | SEQ ID NO: 6212 |
| rs3115635 | SEQ ID NO: 1481 | SEQ ID NO: 3847 | SEQ ID NO: 6213 |
| rs2524172 | SEQ ID NO: 1482 | SEQ ID NO: 3848 | SEQ ID NO: 6214 |
| rs2523746 | SEQ ID NO: 1483 | SEQ ID NO: 3849 | SEQ ID NO: 6215 |
| rs1362069 | SEQ ID NO: 1484 | SEQ ID NO: 3850 | SEQ ID NO: 6216 |
| rs3129203 | SEQ ID NO: 1485 | SEQ ID NO: 3851 | SEQ ID NO: 6217 |
| rs3871248 | SEQ ID NO: 1486 | SEQ ID NO: 3852 | SEQ ID NO: 6218 |
| rs4248157 | SEQ ID NO: 1487 | SEQ ID NO: 3853 | SEQ ID NO: 6219 |
| rs3130140 | SEQ ID NO: 1488 | SEQ ID NO: 3854 | SEQ ID NO: 6220 |
| rs2524100 | SEQ ID NO: 1489 | SEQ ID NO: 3855 | SEQ ID NO: 6221 |
| rs1800625 | SEQ ID NO: 1490 | SEQ ID NO: 3856 | SEQ ID NO: 6222 |
| rs2239709 | SEQ ID NO: 1491 | SEQ ID NO: 3857 | SEQ ID NO: 6223 |
| rs241439 | SEQ ID NO: 1492 | SEQ ID NO: 3858 | SEQ ID NO: 6224 |
| rs6907291 | SEQ ID NO: 1493 | SEQ ID NO: 3859 | SEQ ID NO: 6225 |
| rs6917477 | SEQ ID NO: 1494 | SEQ ID NO: 3860 | SEQ ID NO: 6226 |
| rs6923504 | SEQ ID NO: 1495 | SEQ ID NO: 3861 | SEQ ID NO: 6227 |
| rs6906021 | SEQ ID NO: 1496 | SEQ ID NO: 3862 | SEQ ID NO: 6228 |
| rs2849015 | SEQ ID NO: 1497 | SEQ ID NO: 3863 | SEQ ID NO: 6229 |
| rs2517462 | SEQ ID NO: 1498 | SEQ ID NO: 3864 | SEQ ID NO: 6230 |
| rs7382297 | SEQ ID NO: 1499 | SEQ ID NO: 3865 | SEQ ID NO: 6231 |
| rs6457713 | SEQ ID NO: 1500 | SEQ ID NO: 3866 | SEQ ID NO: 6232 |
| rs1075496 | SEQ ID NO: 1501 | SEQ ID NO: 3867 | SEQ ID NO: 6233 |
| rs3131083 | SEQ ID NO: 1502 | SEQ ID NO: 3868 | SEQ ID NO: 6234 |
| rs2256965 | SEQ ID NO: 1503 | SEQ ID NO: 3869 | SEQ ID NO: 6235 |
| rs3093662 | SEQ ID NO: 1504 | SEQ ID NO: 3870 | SEQ ID NO: 6236 |
| rs2395305 | SEQ ID NO: 1505 | SEQ ID NO: 3871 | SEQ ID NO: 6237 |
| rs3763304 | SEQ ID NO: 1506 | SEQ ID NO: 3872 | SEQ ID NO: 6238 |
| rs2227126 | SEQ ID NO: 1507 | SEQ ID NO: 3873 | SEQ ID NO: 6239 |
| rs261946 | SEQ ID NO: 1508 | SEQ ID NO: 3874 | SEQ ID NO: 6240 |
| rs4987411 | SEQ ID NO: 1509 | SEQ ID NO: 3875 | SEQ ID NO: 6241 |
| rs1633017 | SEQ ID NO: 1510 | SEQ ID NO: 3876 | SEQ ID NO: 6242 |
| rs7382061 | SEQ ID NO: 1511 | SEQ ID NO: 3877 | SEQ ID NO: 6243 |
| rs3129980 | SEQ ID NO: 1512 | SEQ ID NO: 3878 | SEQ ID NO: 6244 |
| rs3734838 | SEQ ID NO: 1513 | SEQ ID NO: 3879 | SEQ ID NO: 6245 |
| rs3129073 | SEQ ID NO: 1514 | SEQ ID NO: 3880 | SEQ ID NO: 6246 |
| rs1063355 | SEQ ID NO: 1515 | SEQ ID NO: 3881 | SEQ ID NO: 6247 |
| rs994321 | SEQ ID NO: 1516 | SEQ ID NO: 3882 | SEQ ID NO: 6248 |
| rs3129809 | SEQ ID NO: 1517 | SEQ ID NO: 3883 | SEQ ID NO: 6249 |
| rs7766854 | SEQ ID NO: 1518 | SEQ ID NO: 3884 | SEQ ID NO: 6250 |
| rs3763288 | SEQ ID NO: 1519 | SEQ ID NO: 3885 | SEQ ID NO: 6251 |
| rs630379 | SEQ ID NO: 1520 | SEQ ID NO: 3886 | SEQ ID NO: 6252 |
| rs2844845 | SEQ ID NO: 1521 | SEQ ID NO: 3887 | SEQ ID NO: 6253 |
| rs3130666 | SEQ ID NO: 1522 | SEQ ID NO: 3888 | SEQ ID NO: 6254 |
| rs2021723 | SEQ ID NO: 1523 | SEQ ID NO: 3889 | SEQ ID NO: 6255 |
| rs1634718 | SEQ ID NO: 1524 | SEQ ID NO: 3890 | SEQ ID NO: 6256 |
| rs1264539 | SEQ ID NO: 1525 | SEQ ID NO: 3891 | SEQ ID NO: 6257 |
| rs2844748 | SEQ ID NO: 1526 | SEQ ID NO: 3892 | SEQ ID NO: 6258 |
| rs805292 | SEQ ID NO: 1527 | SEQ ID NO: 3893 | SEQ ID NO: 6259 |
| rs7741884 | SEQ ID NO: 1528 | SEQ ID NO: 3894 | SEQ ID NO: 6260 |
| rs2245420 | SEQ ID NO: 1529 | SEQ ID NO: 3895 | SEQ ID NO: 6261 |
| rs259940 | SEQ ID NO: 1530 | SEQ ID NO: 3896 | SEQ ID NO: 6262 |
| rs2853935 | SEQ ID NO: 1531 | SEQ ID NO: 3897 | SEQ ID NO: 6263 |
| rs6911467 | SEQ ID NO: 1532 | SEQ ID NO: 3898 | SEQ ID NO: 6264 |
| rs3132712 | SEQ ID NO: 1533 | SEQ ID NO: 3899 | SEQ ID NO: 6265 |
| rs3763317 | SEQ ID NO: 1534 | SEQ ID NO: 3900 | SEQ ID NO: 6266 |
| rs2853939 | SEQ ID NO: 1535 | SEQ ID NO: 3901 | SEQ ID NO: 6267 |
| rs605203 | SEQ ID NO: 1536 | SEQ ID NO: 3902 | SEQ ID NO: 6268 |
| rs6457594 | SEQ ID NO: 1537 | SEQ ID NO: 3903 | SEQ ID NO: 6269 |
| rs2144014 | SEQ ID NO: 1538 | SEQ ID NO: 3904 | SEQ ID NO: 6270 |
| rs2534790 | SEQ ID NO: 1539 | SEQ ID NO: 3905 | SEQ ID NO: 6271 |
| rs986522 | SEQ ID NO: 1540 | SEQ ID NO: 3906 | SEQ ID NO: 6272 |
| rs805264 | SEQ ID NO: 1541 | SEQ ID NO: 3907 | SEQ ID NO: 6273 |
| rs7757269 | SEQ ID NO: 1542 | SEQ ID NO: 3908 | SEQ ID NO: 6274 |
| rs916570 | SEQ ID NO: 1543 | SEQ ID NO: 3909 | SEQ ID NO: 6275 |
| rs3093668 | SEQ ID NO: 1544 | SEQ ID NO: 3910 | SEQ ID NO: 6276 |
| rs928824 | SEQ ID NO: 1545 | SEQ ID NO: 3911 | SEQ ID NO: 6277 |
| rs3132682 | SEQ ID NO: 1546 | SEQ ID NO: 3912 | SEQ ID NO: 6278 |
| rs4248148 | SEQ ID NO: 1547 | SEQ ID NO: 3913 | SEQ ID NO: 6279 |
| rs707928 | SEQ ID NO: 1548 | SEQ ID NO: 3914 | SEQ ID NO: 6280 |
| rs3130375 | SEQ ID NO: 1549 | SEQ ID NO: 3915 | SEQ ID NO: 6281 |
| rs3097644 | SEQ ID NO: 1550 | SEQ ID NO: 3916 | SEQ ID NO: 6282 |
| rs1233397 | SEQ ID NO: 1551 | SEQ ID NO: 3917 | SEQ ID NO: 6283 |
| rs2023371 | SEQ ID NO: 1552 | SEQ ID NO: 3918 | SEQ ID NO: 6284 |
| rs2071540 | SEQ ID NO: 1553 | SEQ ID NO: 3919 | SEQ ID NO: 6285 |
| rs4713603 | SEQ ID NO: 1554 | SEQ ID NO: 3920 | SEQ ID NO: 6286 |
| rs3094014 | SEQ ID NO: 1555 | SEQ ID NO: 3921 | SEQ ID NO: 6287 |
| rs2856726 | SEQ ID NO: 1556 | SEQ ID NO: 3922 | SEQ ID NO: 6288 |
| rs2735005 | SEQ ID NO: 1557 | SEQ ID NO: 3923 | SEQ ID NO: 6289 |
| rs241414 | SEQ ID NO: 1558 | SEQ ID NO: 3924 | SEQ ID NO: 6290 |
| rs2516400 | SEQ ID NO: 1559 | SEQ ID NO: 3925 | SEQ ID NO: 6291 |
| rs2517416 | SEQ ID NO: 1560 | SEQ ID NO: 3926 | SEQ ID NO: 6292 |
| rs3749971 | SEQ ID NO: 1561 | SEQ ID NO: 3927 | SEQ ID NO: 6293 |
| rs6928431 | SEQ ID NO: 1562 | SEQ ID NO: 3928 | SEQ ID NO: 6294 |
| rs6911693 | SEQ ID NO: 1563 | SEQ ID NO: 3929 | SEQ ID NO: 6295 |
| rs1269852 | SEQ ID NO: 1564 | SEQ ID NO: 3930 | SEQ ID NO: 6296 |
| rs241457 | SEQ ID NO: 1565 | SEQ ID NO: 3931 | SEQ ID NO: 6297 |
| rs2077102 | SEQ ID NO: 1566 | SEQ ID NO: 3932 | SEQ ID NO: 6298 |
| rs1736995 | SEQ ID NO: 1567 | SEQ ID NO: 3933 | SEQ ID NO: 6299 |
| rs241413 | SEQ ID NO: 1568 | SEQ ID NO: 3934 | SEQ ID NO: 6300 |
| rs1362074 | SEQ ID NO: 1569 | SEQ ID NO: 3935 | SEQ ID NO: 6301 |
| rs2524222 | SEQ ID NO: 1570 | SEQ ID NO: 3936 | SEQ ID NO: 6302 |
| rs2857768 | SEQ ID NO: 1571 | SEQ ID NO: 3937 | SEQ ID NO: 6303 |
| rs2394390 | SEQ ID NO: 1572 | SEQ ID NO: 3938 | SEQ ID NO: 6304 |
| rs3218819 | SEQ ID NO: 1573 | SEQ ID NO: 3939 | SEQ ID NO: 6305 |
| rs2736176 | SEQ ID NO: 1574 | SEQ ID NO: 3940 | SEQ ID NO: 6306 |
| rs885933 | SEQ ID NO: 1575 | SEQ ID NO: 3941 | SEQ ID NO: 6307 |
| rs2517889 | SEQ ID NO: 1576 | SEQ ID NO: 3942 | SEQ ID NO: 6308 |
| rs2207337 | SEQ ID NO: 1577 | SEQ ID NO: 3943 | SEQ ID NO: 6309 |
| rs4713293 | SEQ ID NO: 1578 | SEQ ID NO: 3944 | SEQ ID NO: 6310 |
| rs2040410 | SEQ ID NO: 1579 | SEQ ID NO: 3945 | SEQ ID NO: 6311 |
| rs1557611 | SEQ ID NO: 1580 | SEQ ID NO: 3946 | SEQ ID NO: 6312 |
| rs3129942 | SEQ ID NO: 1581 | SEQ ID NO: 3947 | SEQ ID NO: 6313 |
| rs3094138 | SEQ ID NO: 1582 | SEQ ID NO: 3948 | SEQ ID NO: 6314 |
| rs3129304 | SEQ ID NO: 1583 | SEQ ID NO: 3949 | SEQ ID NO: 6315 |
| rs2071288 | SEQ ID NO: 1584 | SEQ ID NO: 3950 | SEQ ID NO: 6316 |
| rs6921644 | SEQ ID NO: 1585 | SEQ ID NO: 3951 | SEQ ID NO: 6317 |
| rs206777 | SEQ ID NO: 1586 | SEQ ID NO: 3952 | SEQ ID NO: 6318 |
| rs17719 | SEQ ID NO: 1587 | SEQ ID NO: 3953 | SEQ ID NO: 6319 |
| rs1024470 | SEQ ID NO: 1588 | SEQ ID NO: 3954 | SEQ ID NO: 6320 |
| rs620202 | SEQ ID NO: 1589 | SEQ ID NO: 3955 | SEQ ID NO: 6321 |
| rs409558 | SEQ ID NO: 1590 | SEQ ID NO: 3956 | SEQ ID NO: 6322 |
| rs3117330 | SEQ ID NO: 1591 | SEQ ID NO: 3957 | SEQ ID NO: 6323 |
| rs2071481 | SEQ ID NO: 1592 | SEQ ID NO: 3958 | SEQ ID NO: 6324 |
| rs2071544 | SEQ ID NO: 1593 | SEQ ID NO: 3959 | SEQ ID NO: 6325 |
| rs362512 | SEQ ID NO: 1594 | SEQ ID NO: 3960 | SEQ ID NO: 6326 |
| rs1056032 | SEQ ID NO: 1595 | SEQ ID NO: 3961 | SEQ ID NO: 6327 |
| rs4711185 | SEQ ID NO: 1596 | SEQ ID NO: 3962 | SEQ ID NO: 6328 |
| rs6457204 | SEQ ID NO: 1597 | SEQ ID NO: 3963 | SEQ ID NO: 6329 |
| rs7451190 | SEQ ID NO: 1598 | SEQ ID NO: 3964 | SEQ ID NO: 6330 |
| rs474534 | SEQ ID NO: 1599 | SEQ ID NO: 3965 | SEQ ID NO: 6331 |
| rs1480380 | SEQ ID NO: 1600 | SEQ ID NO: 3966 | SEQ ID NO: 6332 |
| rs6914285 | SEQ ID NO: 1601 | SEQ ID NO: 3967 | SEQ ID NO: 6333 |
| rs3129953 | SEQ ID NO: 1602 | SEQ ID NO: 3968 | SEQ ID NO: 6334 |
| rs6927487 | SEQ ID NO: 1603 | SEQ ID NO: 3969 | SEQ ID NO: 6335 |
| rs3095151 | SEQ ID NO: 1604 | SEQ ID NO: 3970 | SEQ ID NO: 6336 |
| rs1573298 | SEQ ID NO: 1605 | SEQ ID NO: 3971 | SEQ ID NO: 6337 |
| rs3104395 | SEQ ID NO: 1606 | SEQ ID NO: 3972 | SEQ ID NO: 6338 |
| rs2395316 | SEQ ID NO: 1607 | SEQ ID NO: 3973 | SEQ ID NO: 6339 |
| rs1264707 | SEQ ID NO: 1608 | SEQ ID NO: 3974 | SEQ ID NO: 6340 |
| rs3095313 | SEQ ID NO: 1609 | SEQ ID NO: 3975 | SEQ ID NO: 6341 |
| rs2735054 | SEQ ID NO: 1610 | SEQ ID NO: 3976 | SEQ ID NO: 6342 |
| rs886390 | SEQ ID NO: 1611 | SEQ ID NO: 3977 | SEQ ID NO: 6343 |
| rs2523721 | SEQ ID NO: 1612 | SEQ ID NO: 3978 | SEQ ID NO: 6344 |
| rs2239800 | SEQ ID NO: 1613 | SEQ ID NO: 3979 | SEQ ID NO: 6345 |
| rs2747466 | SEQ ID NO: 1614 | SEQ ID NO: 3980 | SEQ ID NO: 6346 |
| rs1029239 | SEQ ID NO: 1615 | SEQ ID NO: 3981 | SEQ ID NO: 6347 |
| rs2523676 | SEQ ID NO: 1616 | SEQ ID NO: 3982 | SEQ ID NO: 6348 |
| rs3757335 | SEQ ID NO: 1617 | SEQ ID NO: 3983 | SEQ ID NO: 6349 |
| rs760070 | SEQ ID NO: 1618 | SEQ ID NO: 3984 | SEQ ID NO: 6350 |
| rs2442719 | SEQ ID NO: 1619 | SEQ ID NO: 3985 | SEQ ID NO: 6351 |
| rs1573297 | SEQ ID NO: 1620 | SEQ ID NO: 3986 | SEQ ID NO: 6352 |
| rs3132569 | SEQ ID NO: 1621 | SEQ ID NO: 3987 | SEQ ID NO: 6353 |
| rs2844523 | SEQ ID NO: 1622 | SEQ ID NO: 3988 | SEQ ID NO: 6354 |
| rs2077573 | SEQ ID NO: 1623 | SEQ ID NO: 3989 | SEQ ID NO: 6355 |
| rs2249168 | SEQ ID NO: 1624 | SEQ ID NO: 3990 | SEQ ID NO: 6356 |
| rs2844780 | SEQ ID NO: 1625 | SEQ ID NO: 3991 | SEQ ID NO: 6357 |
| rs6457655 | SEQ ID NO: 1626 | SEQ ID NO: 3992 | SEQ ID NO: 6358 |
| rs7770024 | SEQ ID NO: 1627 | SEQ ID NO: 3993 | SEQ ID NO: 6359 |
| rs458434 | SEQ ID NO: 1628 | SEQ ID NO: 3994 | SEQ ID NO: 6360 |
| rs204894 | SEQ ID NO: 1629 | SEQ ID NO: 3995 | SEQ ID NO: 6361 |
| rs6905949 | SEQ ID NO: 1630 | SEQ ID NO: 3996 | SEQ ID NO: 6362 |
| rs3135376 | SEQ ID NO: 1631 | SEQ ID NO: 3997 | SEQ ID NO: 6363 |
| rs1018434 | SEQ ID NO: 1632 | SEQ ID NO: 3998 | SEQ ID NO: 6364 |
| rs204879 | SEQ ID NO: 1633 | SEQ ID NO: 3999 | SEQ ID NO: 6365 |
| rs3129943 | SEQ ID NO: 1634 | SEQ ID NO: 4000 | SEQ ID NO: 6366 |
| rs7772569 | SEQ ID NO: 1635 | SEQ ID NO: 4001 | SEQ ID NO: 6367 |
| rs2248902 | SEQ ID NO: 1636 | SEQ ID NO: 4002 | SEQ ID NO: 6368 |
| rs2517449 | SEQ ID NO: 1637 | SEQ ID NO: 4003 | SEQ ID NO: 6369 |
| rs2859448 | SEQ ID NO: 1638 | SEQ ID NO: 4004 | SEQ ID NO: 6370 |
| rs2857694 | SEQ ID NO: 1639 | SEQ ID NO: 4005 | SEQ ID NO: 6371 |
| rs3132671 | SEQ ID NO: 1640 | SEQ ID NO: 4006 | SEQ ID NO: 6372 |
| rs2395301 | SEQ ID NO: 1641 | SEQ ID NO: 4007 | SEQ ID NO: 6373 |
| rs2516455 | SEQ ID NO: 1642 | SEQ ID NO: 4008 | SEQ ID NO: 6374 |
| rs2294472 | SEQ ID NO: 1643 | SEQ ID NO: 4009 | SEQ ID NO: 6375 |
| rs2077580 | SEQ ID NO: 1644 | SEQ ID NO: 4010 | SEQ ID NO: 6376 |
| rs1061783 | SEQ ID NO: 1645 | SEQ ID NO: 4011 | SEQ ID NO: 6377 |
| rs3135024 | SEQ ID NO: 1646 | SEQ ID NO: 4012 | SEQ ID NO: 6378 |
| rs204994 | SEQ ID NO: 1647 | SEQ ID NO: 4013 | SEQ ID NO: 6379 |
| rs1059174 | SEQ ID NO: 1648 | SEQ ID NO: 4014 | SEQ ID NO: 6380 |
| rs1233422 | SEQ ID NO: 1649 | SEQ ID NO: 4015 | SEQ ID NO: 6381 |
| rs7772297 | SEQ ID NO: 1650 | SEQ ID NO: 4016 | SEQ ID NO: 6382 |
| rs3132613 | SEQ ID NO: 1651 | SEQ ID NO: 4017 | SEQ ID NO: 6383 |
| rs2272592 | SEQ ID NO: 1652 | SEQ ID NO: 4018 | SEQ ID NO: 6384 |
| rs2523790 | SEQ ID NO: 1653 | SEQ ID NO: 4019 | SEQ ID NO: 6385 |
| rs2071476 | SEQ ID NO: 1654 | SEQ ID NO: 4020 | SEQ ID NO: 6386 |
| rs2105903 | SEQ ID NO: 1655 | SEQ ID NO: 4021 | SEQ ID NO: 6387 |
| rs7382555 | SEQ ID NO: 1656 | SEQ ID NO: 4022 | SEQ ID NO: 6388 |
| rs3115663 | SEQ ID NO: 1657 | SEQ ID NO: 4023 | SEQ ID NO: 6389 |
| rs492899 | SEQ ID NO: 1658 | SEQ ID NO: 4024 | SEQ ID NO: 6390 |
| rs707939 | SEQ ID NO: 1659 | SEQ ID NO: 4025 | SEQ ID NO: 6391 |
| rs3094614 | SEQ ID NO: 1660 | SEQ ID NO: 4026 | SEQ ID NO: 6392 |
| rs2734990 | SEQ ID NO: 1661 | SEQ ID NO: 4027 | SEQ ID NO: 6393 |
| rs2273612 | SEQ ID NO: 1662 | SEQ ID NO: 4028 | SEQ ID NO: 6394 |
| rs438999 | SEQ ID NO: 1663 | SEQ ID NO: 4029 | SEQ ID NO: 6395 |
| rs206786 | SEQ ID NO: 1664 | SEQ ID NO: 4030 | SEQ ID NO: 6396 |
| rs362536 | SEQ ID NO: 1665 | SEQ ID NO: 4031 | SEQ ID NO: 6397 |
| rs2523395 | SEQ ID NO: 1666 | SEQ ID NO: 4032 | SEQ ID NO: 6398 |
| rs1064190 | SEQ ID NO: 1667 | SEQ ID NO: 4033 | SEQ ID NO: 6399 |
| rs3130267 | SEQ ID NO: 1668 | SEQ ID NO: 4034 | SEQ ID NO: 6400 |
| rs7762933 | SEQ ID NO: 1669 | SEQ ID NO: 4035 | SEQ ID NO: 6401 |
| rs3094211 | SEQ ID NO: 1670 | SEQ ID NO: 4036 | SEQ ID NO: 6402 |
| rs3129932 | SEQ ID NO: 1671 | SEQ ID NO: 4037 | SEQ ID NO: 6403 |
| rs2275855 | SEQ ID NO: 1672 | SEQ ID NO: 4038 | SEQ ID NO: 6404 |
| rs2516399 | SEQ ID NO: 1673 | SEQ ID NO: 4039 | SEQ ID NO: 6405 |
| rs2857210 | SEQ ID NO: 1674 | SEQ ID NO: 4040 | SEQ ID NO: 6406 |
| rs7357044 | SEQ ID NO: 1675 | SEQ ID NO: 4041 | SEQ ID NO: 6407 |
| rs592229 | SEQ ID NO: 1676 | SEQ ID NO: 4042 | SEQ ID NO: 6408 |
| rs2844773 | SEQ ID NO: 1677 | SEQ ID NO: 4043 | SEQ ID NO: 6409 |
| rs2523459 | SEQ ID NO: 1678 | SEQ ID NO: 4044 | SEQ ID NO: 6410 |
| rs2734962 | SEQ ID NO: 1679 | SEQ ID NO: 4045 | SEQ ID NO: 6411 |
| rs2894046 | SEQ ID NO: 1680 | SEQ ID NO: 4046 | SEQ ID NO: 6412 |
| rs15574 | SEQ ID NO: 1681 | SEQ ID NO: 4047 | SEQ ID NO: 6413 |
| rs6919855 | SEQ ID NO: 1682 | SEQ ID NO: 4048 | SEQ ID NO: 6414 |
| rs1362076 | SEQ ID NO: 1683 | SEQ ID NO: 4049 | SEQ ID NO: 6415 |
| rs740884 | SEQ ID NO: 1684 | SEQ ID NO: 4050 | SEQ ID NO: 6416 |
| rs6922512 | SEQ ID NO: 1685 | SEQ ID NO: 4051 | SEQ ID NO: 6417 |
| rs6926737 | SEQ ID NO: 1686 | SEQ ID NO: 4052 | SEQ ID NO: 6418 |
| rs2523747 | SEQ ID NO: 1687 | SEQ ID NO: 4053 | SEQ ID NO: 6419 |
| rs2023470 | SEQ ID NO: 1688 | SEQ ID NO: 4054 | SEQ ID NO: 6420 |
| rs2239839 | SEQ ID NO: 1689 | SEQ ID NO: 4055 | SEQ ID NO: 6421 |
| rs3131050 | SEQ ID NO: 1690 | SEQ ID NO: 4056 | SEQ ID NO: 6422 |
| rs2523984 | SEQ ID NO: 1691 | SEQ ID NO: 4057 | SEQ ID NO: 6423 |
| rs3132965 | SEQ ID NO: 1692 | SEQ ID NO: 4058 | SEQ ID NO: 6424 |
| rs3115537 | SEQ ID NO: 1693 | SEQ ID NO: 4059 | SEQ ID NO: 6425 |
| rs2647044 | SEQ ID NO: 1694 | SEQ ID NO: 4060 | SEQ ID NO: 6426 |
| rs1419640 | SEQ ID NO: 1695 | SEQ ID NO: 4061 | SEQ ID NO: 6427 |
| rs8283 | SEQ ID NO: 1696 | SEQ ID NO: 4062 | SEQ ID NO: 6428 |
| rs2517810 | SEQ ID NO: 1697 | SEQ ID NO: 4063 | SEQ ID NO: 6429 |
| rs2394694 | SEQ ID NO: 1698 | SEQ ID NO: 4064 | SEQ ID NO: 6430 |
| rs2076483 | SEQ ID NO: 1699 | SEQ ID NO: 4065 | SEQ ID NO: 6431 |
| rs411337 | SEQ ID NO: 1700 | SEQ ID NO: 4066 | SEQ ID NO: 6432 |
| rs6934993 | SEQ ID NO: 1701 | SEQ ID NO: 4067 | SEQ ID NO: 6433 |
| rs1245371 | SEQ ID NO: 1702 | SEQ ID NO: 4068 | SEQ ID NO: 6434 |
| rs6902723 | SEQ ID NO: 1703 | SEQ ID NO: 4069 | SEQ ID NO: 6435 |
| rs3134942 | SEQ ID NO: 1704 | SEQ ID NO: 4070 | SEQ ID NO: 6436 |
| rs454212 | SEQ ID NO: 1705 | SEQ ID NO: 4071 | SEQ ID NO: 6437 |
| rs1362126 | SEQ ID NO: 1706 | SEQ ID NO: 4072 | SEQ ID NO: 6438 |
| rs2075701 | SEQ ID NO: 1707 | SEQ ID NO: 4073 | SEQ ID NO: 6439 |
| rs3130502 | SEQ ID NO: 1708 | SEQ ID NO: 4074 | SEQ ID NO: 6440 |
| rs1057373 | SEQ ID NO: 1709 | SEQ ID NO: 4075 | SEQ ID NO: 6441 |
| rs1736936 | SEQ ID NO: 1710 | SEQ ID NO: 4076 | SEQ ID NO: 6442 |
| rs7763159 | SEQ ID NO: 1711 | SEQ ID NO: 4077 | SEQ ID NO: 6443 |
| rs2517755 | SEQ ID NO: 1712 | SEQ ID NO: 4078 | SEQ ID NO: 6444 |
| rs3131000 | SEQ ID NO: 1713 | SEQ ID NO: 4079 | SEQ ID NO: 6445 |
| rs1233384 | SEQ ID NO: 1714 | SEQ ID NO: 4080 | SEQ ID NO: 6446 |
| rs2239804 | SEQ ID NO: 1715 | SEQ ID NO: 4081 | SEQ ID NO: 6447 |
| rs3132668 | SEQ ID NO: 1716 | SEQ ID NO: 4082 | SEQ ID NO: 6448 |
| rs2074464 | SEQ ID NO: 1717 | SEQ ID NO: 4083 | SEQ ID NO: 6449 |
| rs2857107 | SEQ ID NO: 1718 | SEQ ID NO: 4084 | SEQ ID NO: 6450 |
| rs1362063 | SEQ ID NO: 1719 | SEQ ID NO: 4085 | SEQ ID NO: 6451 |
| rs1536215 | SEQ ID NO: 1720 | SEQ ID NO: 4086 | SEQ ID NO: 6452 |
| rs3135395 | SEQ ID NO: 1721 | SEQ ID NO: 4087 | SEQ ID NO: 6453 |
| rs3757328 | SEQ ID NO: 1722 | SEQ ID NO: 4088 | SEQ ID NO: 6454 |
| rs3130982 | SEQ ID NO: 1723 | SEQ ID NO: 4089 | SEQ ID NO: 6455 |
| rs2395269 | SEQ ID NO: 1724 | SEQ ID NO: 4090 | SEQ ID NO: 6456 |
| rs1264423 | SEQ ID NO: 1725 | SEQ ID NO: 4091 | SEQ ID NO: 6457 |
| rs2532929 | SEQ ID NO: 1726 | SEQ ID NO: 4092 | SEQ ID NO: 6458 |
| rs805303 | SEQ ID NO: 1727 | SEQ ID NO: 4093 | SEQ ID NO: 6459 |
| rs2260000 | SEQ ID NO: 1728 | SEQ ID NO: 4094 | SEQ ID NO: 6460 |
| rs86567 | SEQ ID NO: 1729 | SEQ ID NO: 4095 | SEQ ID NO: 6461 |
| rs3117328 | SEQ ID NO: 1730 | SEQ ID NO: 4096 | SEQ ID NO: 6462 |
| rs4711203 | SEQ ID NO: 1731 | SEQ ID NO:.4097 | SEQ ID NO: 6463 |
| rs2763982 | SEQ ID NO: 1732 | SEQ ID NO: 4098 | SEQ ID NO: 6464 |
| rs3130349 | SEQ ID NO: 1733 | SEQ ID NO: 4099 | SEQ ID NO: 6465 |
| rs2021365 | SEQ ID NO: 1734 | SEQ ID NO: 4100 | SEQ ID NO: 6466 |
| rs3117231 | SEQ ID NO: 1735 | SEQ ID NO: 4101 | SEQ ID NO: 6467 |
| rs2021719 | SEQ ID NO: 1736 | SEQ ID NO: 4102 | SEQ ID NO: 6468 |
| rs5009557 | SEQ ID NO: 1737 | SEQ ID NO: 4103 | SEQ ID NO: 6469 |
| rs1269556 | SEQ ID NO: 1738 | SEQ ID NO: 4104 | SEQ ID NO: 6470 |
| rs3131785 | SEQ ID NO: 1739 | SEQ ID NO: 4105 | SEQ ID NO: 6471 |
| rs362521 | SEQ ID NO: 1740 | SEQ ID NO: 4106 | SEQ ID NO: 6472 |
| rs644774 | SEQ ID NO: 1741 | SEQ ID NO: 4107 | SEQ ID NO: 6473 |
| rs558702 | SEQ ID NO: 1742 | SEQ ID NO: 4108 | SEQ ID NO: 6474 |
| rs3132571 | SEQ ID NO: 1743 | SEQ ID NO: 4109 | SEQ ID NO: 6475 |
| rs6932290 | SEQ ID NO: 1744 | SEQ ID NO: 4110 | SEQ ID NO: 6476 |
| rs429608 | SEQ ID NO: 1745 | SEQ ID NO: 4111 | SEQ ID NO: 6477 |
| rs2245738 | SEQ ID NO: 1746 | SEQ ID NO: 4112 | SEQ ID NO: 6478 |
| rs1633034 | SEQ ID NO: 1747 | SEQ ID NO: 4113 | SEQ ID NO: 6479 |
| rs2524179 | SEQ ID NO: 1748 | SEQ ID NO: 4114 | SEQ ID NO: 6480 |
| rs7776164 | SEQ ID NO: 1749 | SEQ ID NO: 4115 | SEQ ID NO: 6481 |
| rs3095226 | SEQ ID NO: 1750 | SEQ ID NO: 4116 | SEQ ID NO: 6482 |
| rs6457044 | SEQ ID NO: 1751 | SEQ ID NO: 4117 | SEQ ID NO: 6483 |
| rs362515 | SEQ ID NO: 1752 | SEQ ID NO: 4118 | SEQ ID NO: 6484 |
| rs3131934 | SEQ ID NO: 1753 | SEQ ID NO: 4119 | SEQ ID NO: 6485 |
| rs750332 | SEQ ID NO: 1754 | SEQ ID NO: 4120 | SEQ ID NO: 6486 |
| rs5009448 | SEQ ID NO: 1755 | SEQ ID NO: 4121 | SEQ ID NO: 6487 |
| rs2508024 | SEQ ID NO: 1756 | SEQ ID NO: 4122 | SEQ ID NO: 6488 |
| rs3130275 | SEQ ID NO: 1757 | SEQ ID NO: 4123 | SEQ ID NO: 6489 |
| rs3131115 | SEQ ID NO: 1758 | SEQ ID NO: 4124 | SEQ ID NO: 6490 |
| rs1131541 | SEQ ID NO: 1759 | SEQ ID NO: 4125 | SEQ ID NO: 6491 |
| rs7452206 | SEQ ID NO: 1760 | SEQ ID NO: 4126 | SEQ ID NO: 6492 |
| rs2074470 | SEQ ID NO: 1761 | SEQ ID NO: 4127 | SEQ ID NO: 6493 |
| rs7751953 | SEQ ID NO: 1762 | SEQ ID NO: 4128 | SEQ ID NO: 6494 |
| rs2073148 | SEQ ID NO: 1763 | SEQ ID NO: 4129 | SEQ ID NO: 6495 |
| rs885910 | SEQ ID NO: 1764 | SEQ ID NO: 4130 | SEQ ID NO: 6496 |
| rs3094179 | SEQ ID NO: 1765 | SEQ ID NO: 4131 | SEQ ID NO: 6497 |
| rs2301277 | SEQ ID NO: 1766 | SEQ ID NO: 4132 | SEQ ID NO: 6498 |
| rs2239885 | SEQ ID NO: 1767 | SEQ ID NO: 4133 | SEQ ID NO: 6499 |
| rs356963 | SEQ ID NO: 1768 | SEQ ID NO: 4134 | SEQ ID NO: 6500 |
| rs3132721 | SEQ ID NO: 1769 | SEQ ID NO: 4135 | SEQ ID NO: 6501 |
| rs928976 | SEQ ID NO: 1770 | SEQ ID NO: 4136 | SEQ ID NO: 6502 |
| rs3131784 | SEQ ID NO: 1771 | SEQ ID NO: 4137 | SEQ ID NO: 6503 |
| rs3097652 | SEQ ID NO: 1772 | SEQ ID NO: 4138 | SEQ ID NO: 6504 |
| rs805304 | SEQ ID NO: 1773 | SEQ ID NO: 4139 | SEQ ID NO: 6505 |
| rs5017401 | SEQ ID NO: 1774 | SEQ ID NO: 4140 | SEQ ID NO: 6506 |
| rs130067 | SEQ ID NO: 1775 | SEQ ID NO: 4141 | SEQ ID NO: 6507 |
| rs2530716 | SEQ ID NO: 1776 | SEQ ID NO: 4142 | SEQ ID NO: 6508 |
| rs635348 | SEQ ID NO: 1777 | SEQ ID NO: 4143 | SEQ ID NO: 6509 |
| rs464865 | SEQ ID NO: 1778 | SEQ ID NO: 4144 | SEQ ID NO: 6510 |
| rs2844688 | SEQ ID NO: 1779 | SEQ ID NO: 4145 | SEQ ID NO: 6511 |
| rs1632938 | SEQ ID NO: 1780 | SEQ ID NO: 4146 | SEQ ID NO: 6512 |
| rs565876 | SEQ ID NO: 1781 | SEQ ID NO: 4147 | SEQ ID NO: 6513 |
| rs6911246 | SEQ ID NO: 1782 | SEQ ID NO: 4148 | SEQ ID NO: 6514 |
| rs3093557 | SEQ ID NO: 1783 | SEQ ID NO: 4149 | SEQ ID NO: 6515 |
| rs2516672 | SEQ ID NO: 1784 | SEQ ID NO: 4150 | SEQ ID NO: 6516 |
| rs3095307 | SEQ ID NO: 1785 | SEQ ID NO: 4151 | SEQ ID NO: 6517 |
| rs3814959 | SEQ ID NO: 1786 | SEQ ID NO: 4152 | SEQ ID NO: 6518 |
| rs1611493 | SEQ ID NO: 1787 | SEQ ID NO: 4153 | SEQ ID NO: 6519 |
| rs1233492 | SEQ ID NO: 1788 | SEQ ID NO: 4154 | SEQ ID NO: 6520 |
| rs6456890 | SEQ ID NO: 1789 | SEQ ID NO: 4155 | SEQ ID NO: 6521 |
| rs2844680 | SEQ ID NO: 1790 | SEQ ID NO: 4156 | SEQ ID NO: 6522 |
| rs769177 | SEQ ID NO: 1791 | SEQ ID NO: 4157 | SEQ ID NO: 6523 |
| rs2844659 | SEQ ID NO: 1792 | SEQ ID NO: 4158 | SEQ ID NO: 6524 |
| rs2516698 | SEQ ID NO: 1793 | SEQ ID NO: 4159 | SEQ ID NO: 6525 |
| rs204890 | SEQ ID NO: 1794 | SEQ ID NO: 4160 | SEQ ID NO: 6526 |
| rs6457699 | SEQ ID NO: 1795 | SEQ ID NO: 4161 | SEQ ID NO: 6527 |
| rs2071787 | SEQ ID NO: 1796 | SEQ ID NO: 4162 | SEQ ID NO: 6528 |
| rs892666 | SEQ ID NO: 1797 | SEQ ID NO: 4163 | SEQ ID NO: 6529 |
| rs2517894 | SEQ ID NO: 1798 | SEQ ID NO: 4164 | SEQ ID NO: 6530 |
| rs1264348 | SEQ ID NO: 1799 | SEQ ID NO: 4165 | SEQ ID NO: 6531 |
| rs2394734 | SEQ ID NO: 1800 | SEQ ID NO: 4166 | SEQ ID NO: 6532 |
| rs3749950 | SEQ ID NO: 1801 | SEQ ID NO: 4167 | SEQ ID NO: 6533 |
| rs1264350 | SEQ ID NO: 1802 | SEQ ID NO: 4168 | SEQ ID NO: 6534 |
| rs2517407 | SEQ ID NO: 1803 | SEQ ID NO: 4169 | SEQ ID NO: 6535 |
| rs3117426 | SEQ ID NO: 1804 | SEQ ID NO: 4170 | SEQ ID NO: 6536 |
| rs3134767 | SEQ ID NO: 1805 | SEQ ID NO: 4171 | SEQ ID NO: 6537 |
| rs2535238 | SEQ ID NO: 1806 | SEQ ID NO: 4172 | SEQ ID NO: 6538 |
| rs805282 | SEQ ID NO: 1807 | SEQ ID NO: 4173 | SEQ ID NO: 6539 |
| rs401618 | SEQ ID NO: 1808 | SEQ ID NO: 4174 | SEQ ID NO: 6540 |
| rs550605 | SEQ ID NO: 1809 | SEQ ID NO: 4175 | SEQ ID NO: 6541 |
| rs2021722 | SEQ ID NO: 1810 | SEQ ID NO: 4176 | SEQ ID NO: 6542 |
| rs885950 | SEQ ID NO: 1811 | SEQ ID NO: 4177 | SEQ ID NO: 6543 |
| rs2516489 | SEQ ID NO: 1812 | SEQ ID NO: 4178 | SEQ ID NO: 6544 |
| rs2239842 | SEQ ID NO: 1813 | SEQ ID NO: 4179 | SEQ ID NO: 6545 |
| rs3117235 | SEQ ID NO: 1814 | SEQ ID NO: 4180 | SEQ ID NO: 6546 |
| rs446145 | SEQ ID NO: 1815 | SEQ ID NO: 4181 | SEQ ID NO: 6547 |
| rs2894260 | SEQ ID NO: 1816 | SEQ ID NO: 4182 | SEQ ID NO: 6548 |
| rs2143468 | SEQ ID NO: 1817 | SEQ ID NO: 4183 | SEQ ID NO: 6549 |
| rs3873260 | SEQ ID NO: 1818 | SEQ ID NO: 4184 | SEQ ID NO: 6550 |
| rs1050391 | SEQ ID NO: 1819 | SEQ ID NO: 4185 | SEQ ID NO: 6551 |
| rs3094001 | SEQ ID NO: 1820 | SEQ ID NO: 4186 | SEQ ID NO: 6552 |
| rs715044 | SEQ ID NO: 1821 | SEQ ID NO: 4187 | SEQ ID NO: 6553 |
| rs1610640 | SEQ ID NO: 1822 | SEQ ID NO: 4188 | SEQ ID NO: 6554 |
| rs887468 | SEQ ID NO: 1823 | SEQ ID NO: 4189 | SEQ ID NO: 6555 |
| rs755714 | SEQ ID NO: 1824 | SEQ ID NO: 4190 | SEQ ID NO: 6556 |
| rs1265777 | SEQ ID NO: 1825 | SEQ ID NO: 4191 | SEQ ID NO: 6557 |
| rs999265 | SEQ ID NO: 1826 | SEQ ID NO: 4192 | SEQ ID NO: 6558 |
| rs2021729 | SEQ ID NO: 1827 | SEQ ID NO: 4193 | SEQ ID NO: 6559 |
| rs7452076 | SEQ ID NO: 1828 | SEQ ID NO: 4194 | SEQ ID NO: 6560 |
| rs743399 | SEQ ID NO: 1829 | SEQ ID NO: 4195 | SEQ ID NO: 6561 |
| rs2256594 | SEQ ID NO: 1830 | SEQ ID NO: 4196 | SEQ ID NO: 6562 |
| rs1046080 | SEQ ID NO: 1831 | SEQ ID NO: 4197 | SEQ ID NO: 6563 |
| rs2523807 | SEQ ID NO: 1832 | SEQ ID NO: 4198 | SEQ ID NO: 6564 |
| rs3094647 | SEQ ID NO: 1833 | SEQ ID NO: 4199 | SEQ ID NO: 6565 |
| rs421446 | SEQ ID NO: 1834 | SEQ ID NO: 4200 | SEQ ID NO: 6566 |
| rs6415110 | SEQ ID NO: 1835 | SEQ ID NO: 4201 | SEQ ID NO: 6567 |
| rs2285805 | SEQ ID NO: 1836 | SEQ ID NO: 4202 | SEQ ID NO: 6568 |
| rs2242955 | SEQ ID NO: 1837 | SEQ ID NO: 4203 | SEQ ID NO: 6569 |
| rs29233 | SEQ ID NO: 1838 | SEQ ID NO: 4204 | SEQ ID NO: 6570 |
| rs4711312 | SEQ ID NO: 1839 | SEQ ID NO: 4205 | SEQ ID NO: 6571 |
| rs1264327 | SEQ ID NO: 1840 | SEQ ID NO: 4206 | SEQ ID NO: 6572 |
| rs2261033 | SEQ ID NO: 1841 | SEQ ID NO: 4207 | SEQ ID NO: 6573 |
| rs805288 | SEQ ID NO: 1842 | SEQ ID NO: 4208 | SEQ ID NO: 6574 |
| rs3106189 | SEQ ID NO: 1843 | SEQ ID NO: 4209 | SEQ ID NO: 6575 |
| rs2157338 | SEQ ID NO: 1844 | SEQ ID NO: 4210 | SEQ ID NO: 6576 |
| rs805299 | SEQ ID NO: 1845 | SEQ ID NO: 4211 | SEQ ID NO: 6577 |
| rs2074505 | SEQ ID NO: 1846 | SEQ ID NO: 4212 | SEQ ID NO: 6578 |
| rs1042338 | SEQ ID NO: 1847 | SEQ ID NO: 4213 | SEQ ID NO: 6579 |
| rs1059510 | SEQ ID NO: 1848 | SEQ ID NO: 4214 | SEQ ID NO: 6580 |
| rs4678 | SEQ ID NO: 1849 | SEQ ID NO: 4215 | SEQ ID NO: 6581 |
| rs396090 | SEQ ID NO: 1850 | SEQ ID NO: 4216 | SEQ ID NO: 6582 |
| rs1233419 | SEQ ID NO: 1851 | SEQ ID NO: 4217 | SEQ ID NO: 6583 |
| rs1265063 | SEQ ID NO: 1852 | SEQ ID NO: 4218 | SEQ ID NO: 6584 |
| rs3117223 | SEQ ID NO: 1853 | SEQ ID NO: 4219 | SEQ ID NO: 6585 |
| rs2269475 | SEQ ID NO: 1854 | SEQ ID NO: 4220 | SEQ ID NO: 6586 |
| rs2235499 | SEQ ID NO: 1855 | SEQ ID NO: 4221 | SEQ ID NO: 6587 |
| rs6457020 | SEQ ID NO: 1856 | SEQ ID NO: 4222 | SEQ ID NO: 6588 |
| rs185819 | SEQ ID NO: 1857 | SEQ ID NO: 4223 | SEQ ID NO: 6589 |
| rs2273611 | SEQ ID NO: 1858 | SEQ ID NO: 4224 | SEQ ID NO: 6590 |
| rs1632879 | SEQ ID NO: 1859 | SEQ ID NO: 4225 | SEQ ID NO: 6591 |
| rs3093551 | SEQ ID NO: 1860 | SEQ ID NO: 4226 | SEQ ID NO: 6592 |
| rs130072 | SEQ ID NO: 1861 | SEQ ID NO: 4227 | SEQ ID NO: 6593 |
| rs2295667 | SEQ ID NO: 1862 | SEQ ID NO: 4228 | SEQ ID NO: 6594 |
| rs2517466 | SEQ ID NO: 1863 | SEQ ID NO: 4229 | SEQ ID NO: 6595 |
| rs2844724 | SEQ ID NO: 1864 | SEQ ID NO: 4230 | SEQ ID NO: 6596 |
| rs7769073 | SEQ ID NO: 1865 | SEQ ID NO: 4231 | SEQ ID NO: 6597 |
| rs3130780 | SEQ ID NO: 1866 | SEQ ID NO: 4232 | SEQ ID NO: 6598 |
| rs4713354 | SEQ ID NO: 1867 | SEQ ID NO: 4233 | SEQ ID NO: 6599 |
| rs2021720 | SEQ ID NO: 1868 | SEQ ID NO: 4234 | SEQ ID NO: 6600 |
| rs6456879 | SEQ ID NO: 1869 | SEQ ID NO: 4235 | SEQ ID NO: 6601 |
| rs6913528 | SEQ ID NO: 1870 | SEQ ID NO: 4236 | SEQ ID NO: 6602 |
| rs6929434 | SEQ ID NO: 1871 | SEQ ID NO: 4237 | SEQ ID NO: 6603 |
| rs2074499 | SEQ ID NO: 1872 | SEQ ID NO: 4238 | SEQ ID NO: 6604 |
| rs518374 | SEQ ID NO: 1873 | SEQ ID NO: 4239 | SEQ ID NO: 6605 |
| rs446735 | SEQ ID NO: 1874 | SEQ ID NO: 4240 | SEQ ID NO: 6606 |
| rs2075799 | SEQ ID NO: 1875 | SEQ ID NO: 4241 | SEQ ID NO: 6607 |
| rs7757012 | SEQ ID NO: 1876 | SEQ ID NO: 4242 | SEQ ID NO: 6608 |
| rs3093984 | SEQ ID NO: 1877 | SEQ ID NO: 4243 | SEQ ID NO: 6609 |
| rs805258 | SEQ ID NO: 1878 | SEQ ID NO: 4244 | SEQ ID NO: 6610 |
| rs2071351 | SEQ ID NO: 1879 | SEQ ID NO: 4245 | SEQ ID NO: 6611 |
| rs6938130 | SEQ ID NO: 1880 | SEQ ID NO: 4246 | SEQ ID NO: 6612 |
| rs3130388 | SEQ ID NO: 1881 | SEQ ID NO: 4247 | SEQ ID NO: 6613 |
| rs1061815 | SEQ ID NO: 1882 | SEQ ID NO: 4248 | SEQ ID NO: 6614 |
| rs241455 | SEQ ID NO: 1883 | SEQ ID NO: 4249 | SEQ ID NO: 6615 |
| rs3891288 | SEQ ID NO: 1884 | SEQ ID NO: 4250 | SEQ ID NO: 6616 |
| rs1655938 | SEQ ID NO: 1885 | SEQ ID NO: 4251 | SEQ ID NO: 6617 |
| rs3131102 | SEQ ID NO: 1886 | SEQ ID NO: 4252 | SEQ ID NO: 6618 |
| rs6457566 | SEQ ID NO: 1887 | SEQ ID NO: 4253 | SEQ ID NO: 6619 |
| rs204887 | SEQ ID NO: 1888 | SEQ ID NO: 4254 | SEQ ID NO: 6620 |
| rs2844725 | SEQ ID NO: 1889 | SEQ ID NO: 4255 | SEQ ID NO: 6621 |
| rs2074491 | SEQ ID NO: 1890 | SEQ ID NO: 4256 | SEQ ID NO: 6622 |
| rs1233425 | SEQ ID NO: 1891 | SEQ ID NO: 4257 | SEQ ID NO: 6623 |
| rs4569 | SEQ ID NO: 1892 | SEQ ID NO: 4258 | SEQ ID NO: 6624 |
| rs2072109 | SEQ ID NO: 1893 | SEQ ID NO: 4259 | SEQ ID NO: 6625 |
| rs3130216 | SEQ ID NO: 1894 | SEQ ID NO: 4260 | SEQ ID NO: 6626 |
| rs3757340 | SEQ ID NO: 1895 | SEQ ID NO: 4261 | SEQ ID NO: 6627 |
| rs3116838 | SEQ ID NO: 1896 | SEQ ID NO: 4262 | SEQ ID NO: 6628 |
| rs7451419 | SEQ ID NO: 1897 | SEQ ID NO: 4263 | SEQ ID NO: 6629 |
| rs1475865 | SEQ ID NO: 1898 | SEQ ID NO: 4264 | SEQ ID NO: 6630 |
| rs1264315 | SEQ ID NO: 1899 | SEQ ID NO: 4265 | SEQ ID NO: 6631 |
| rs2269610 | SEQ ID NO: 1900 | SEQ ID NO: 4266 | SEQ ID NO: 6632 |
| rs3129012 | SEQ ID NO: 1901 | SEQ ID NO: 4267 | SEQ ID NO: 6633 |
| rs3132935 | SEQ ID NO: 1902 | SEQ ID NO: 4268 | SEQ ID NO: 6634 |
| rs2074473 | SEQ ID NO: 1903 | SEQ ID NO: 4269 | SEQ ID NO: 6635 |
| rs3130553 | SEQ ID NO: 1904 | SEQ ID NO: 4270 | SEQ ID NO: 6636 |
| rs2534672 | SEQ ID NO: 1905 | SEQ ID NO: 4271 | SEQ ID NO: 6637 |
| rs1573294 | SEQ ID NO: 1906 | SEQ ID NO: 4272 | SEQ ID NO: 6638 |
| rs7773534 | SEQ ID NO: 1907 | SEQ ID NO: 4273 | SEQ ID NO: 6639 |
| rs1737079 | SEQ ID NO: 1908 | SEQ ID NO: 4274 | SEQ ID NO: 6640 |
| rs805297 | SEQ ID NO: 1909 | SEQ ID NO: 4275 | SEQ ID NO: 6641 |
| rs805298 | SEQ ID NO: 1910 | SEQ ID NO: 4276 | SEQ ID NO: 6642 |
| rs3749951 | SEQ ID NO: 1911 | SEQ ID NO: 4277 | SEQ ID NO: 6643 |
| rs3094208 | SEQ ID NO: 1912 | SEQ ID NO: 4278 | SEQ ID NO: 6644 |
| rs805263 | SEQ ID NO: 1913 | SEQ ID NO: 4279 | SEQ ID NO: 6645 |
| rs3135330 | SEQ ID NO: 1914 | SEQ ID NO: 4280 | SEQ ID NO: 6646 |
| rs734181 | SEQ ID NO: 1915 | SEQ ID NO: 4281 | SEQ ID NO: 6647 |
| rs1050673 | SEQ ID NO: 1916 | SEQ ID NO: 4282 | SEQ ID NO: 6648 |
| rs3129948 | SEQ ID NO: 1917 | SEQ ID NO: 4283 | SEQ ID NO: 6649 |
| rs2517436 | SEQ ID NO: 1918 | SEQ ID NO: 4284 | SEQ ID NO: 6650 |
| rs2242664 | SEQ ID NO: 1919 | SEQ ID NO: 4285 | SEQ ID NO: 6651 |
| rs7761801 | SEQ ID NO: 1920 | SEQ ID NO: 4286 | SEQ ID NO: 6652 |
| rs2077022 | SEQ ID NO: 1921 | SEQ ID NO: 4287 | SEQ ID NO: 6653 |
| rs7743662 | SEQ ID NO: 1922 | SEQ ID NO: 4288 | SEQ ID NO: 6654 |
| rs1264319 | SEQ ID NO: 1923 | SEQ ID NO: 4289 | SEQ ID NO: 6655 |
| rs204990 | SEQ ID NO: 1924 | SEQ ID NO: 4290 | SEQ ID NO: 6656 |
| rs2736158 | SEQ ID NO: 1925 | SEQ ID NO: 4291 | SEQ ID NO: 6657 |
| rs13409 | SEQ ID NO: 1926 | SEQ ID NO: 4292 | SEQ ID NO: 6658 |
| rs3130057 | SEQ ID NO: 1927 | SEQ ID NO: 4293 | SEQ ID NO: 6659 |
| rs2857127 | SEQ ID NO: 1928 | SEQ ID NO: 4294 | SEQ ID NO: 6660 |
| rs7887 | SEQ ID NO: 1929 | SEQ ID NO: 4295 | SEQ ID NO: 6661 |
| rs2535277 | SEQ ID NO: 1930 | SEQ ID NO: 4296 | SEQ ID NO: 6662 |
| rs2395296 | SEQ ID NO: 1931 | SEQ ID NO: 4297 | SEQ ID NO: 6663 |
| rs2294883 | SEQ ID NO: 1932 | SEQ ID NO: 4298 | SEQ ID NO: 6664 |
| rs3129681 | SEQ ID NO: 1933 | SEQ ID NO: 4299 | SEQ ID NO: 6665 |
| rs3130141 | SEQ ID NO: 1934 | SEQ ID NO: 4300 | SEQ ID NO: 6666 |
| rs2285042 | SEQ ID NO: 1935 | SEQ ID NO: 4301 | SEQ ID NO: 6667 |
| rs5027663 | SEQ ID NO: 1936 | SEQ ID NO: 4302 | SEQ ID NO: 6668 |
| rs2294478 | SEQ ID NO: 1937 | SEQ ID NO: 4303 | SEQ ID NO: 6669 |
| rs6457710 | SEQ ID NO: 1938 | SEQ ID NO: 4304 | SEQ ID NO: 6670 |
| rs204900 | SEQ ID NO: 1939 | SEQ ID NO: 4305 | SEQ ID NO: 6671 |
| rs5018660 | SEQ ID NO: 1940 | SEQ ID NO: 4306 | SEQ ID NO: 6672 |
| rs6906587 | SEQ ID NO: 1941 | SEQ ID NO: 4307 | SEQ ID NO: 6673 |
| rs2242653 | SEQ ID NO: 1942 | SEQ ID NO: 4308 | SEQ ID NO: 6674 |
| rs444921 | SEQ ID NO: 1943 | SEQ ID NO: 4309 | SEQ ID NO: 6675 |
| rs2187820 | SEQ ID NO: 1944 | SEQ ID NO: 4310 | SEQ ID NO: 6676 |
| rs2071281 | SEQ ID NO: 1945 | SEQ ID NO: 4311 | SEQ ID NO: 6677 |
| rs3116820 | SEQ ID NO: 1946 | SEQ ID NO: 4312 | SEQ ID NO: 6678 |
| rs241407 | SEQ ID NO: 1947 | SEQ ID NO: 4313 | SEQ ID NO: 6679 |
| rs6457443 | SEQ ID NO: 1948 | SEQ ID NO: 4314 | SEQ ID NO: 6680 |
| rs668558 | SEQ ID NO: 1949 | SEQ ID NO: 4315 | SEQ ID NO: 6681 |
| rs2516675 | SEQ ID NO: 1950 | SEQ ID NO: 4316 | SEQ ID NO: 6682 |
| rs909726 | SEQ ID NO: 1951 | SEQ ID NO: 4317 | SEQ ID NO: 6683 |
| rs4642463 | SEQ ID NO: 1952 | SEQ ID NO: 4318 | SEQ ID NO: 6684 |
| rs3020644 | SEQ ID NO: 1953 | SEQ ID NO: 4319 | SEQ ID NO: 6685 |
| rs2280801 | SEQ ID NO: 1954 | SEQ ID NO: 4320 | SEQ ID NO: 6686 |
| rs1736976 | SEQ ID NO: 1955 | SEQ ID NO: 4321 | SEQ ID NO: 6687 |
| rs1362065 | SEQ ID NO: 1956 | SEQ ID NO: 4322 | SEQ ID NO: 6688 |
| rs3094188 | SEQ ID NO: 1957 | SEQ ID NO: 4323 | SEQ ID NO: 6689 |
| rs7764777 | SEQ ID NO: 1958 | SEQ ID NO: 4324 | SEQ ID NO: 6690 |
| rs4959117 | SEQ ID NO: 1959 | SEQ ID NO: 4325 | SEQ ID NO: 6691 |
| rs2844621 | SEQ ID NO: 1960 | SEQ ID NO: 4326 | SEQ ID NO: 6692 |
| rs3099840 | SEQ ID NO: 1961 | SEQ ID NO: 4327 | SEQ ID NO: 6693 |
| rs2073525 | SEQ ID NO: 1962 | SEQ ID NO: 4328 | SEQ ID NO: 6694 |
| rs3094693 | SEQ ID NO: 1963 | SEQ ID NO: 4329 | SEQ ID NO: 6695 |
| rs7759272 | SEQ ID NO: 1964 | SEQ ID NO: 4330 | SEQ ID NO: 6696 |
| rs6911350 | SEQ ID NO: 1965 | SEQ ID NO: 4331 | SEQ ID NO: 6697 |
| rs2070600 | SEQ ID NO: 1966 | SEQ ID NO: 4332 | SEQ ID NO: 6698 |
| rs2269428 | SEQ ID NO: 1967 | SEQ ID NO: 4333 | SEQ ID NO: 6699 |
| rs2302399 | SEQ ID NO: 1968 | SEQ ID NO: 4334 | SEQ ID NO: 6700 |
| rs1633098 | SEQ ID NO: 1969 | SEQ ID NO: 4335 | SEQ ID NO: 6701 |
| rs555007 | SEQ ID NO: 1970 | SEQ ID NO: 4336 | SEQ ID NO: 6702 |
| rs2508042 | SEQ ID NO: 1971 | SEQ ID NO: 4337 | SEQ ID NO: 6703 |
| rs3130653 | SEQ ID NO: 1972 | SEQ ID NO: 4338 | SEQ ID NO: 6704 |
| rs1705003 | SEQ ID NO: 1973 | SEQ ID NO: 4339 | SEQ ID NO: 6705 |
| rs764554 | SEQ ID NO: 1974 | SEQ ID NO: 4340 | SEQ ID NO: 6706 |
| rs2532942 | SEQ ID NO: 1975 | SEQ ID NO: 4341 | SEQ ID NO: 6707 |
| rs213207 | SEQ ID NO: 1976 | SEQ ID NO: 4342 | SEQ ID NO: 6708 |
| rs2428494 | SEQ ID NO: 1977 | SEQ ID NO: 4343 | SEQ ID NO: 6709 |
| rs1799910 | SEQ ID NO: 1978 | SEQ ID NO: 4344 | SEQ ID NO: 6710 |
| rs2071480 | SEQ ID NO: 1979 | SEQ ID NO: 4345 | SEQ ID NO: 6711 |
| rs1079687 | SEQ ID NO: 1980 | SEQ ID NO: 4346 | SEQ ID NO: 6712 |
| rs970902 | SEQ ID NO: 1981 | SEQ ID NO: 4347 | SEQ ID NO: 6713 |
| rs6900438 | SEQ ID NO: 1982 | SEQ ID NO: 4348 | SEQ ID NO: 6714 |
| rs213213 | SEQ ID NO: 1983 | SEQ ID NO: 4349 | SEQ ID NO: 6715 |
| rs410909 | SEQ ID NO: 1984 | SEQ ID NO: 4350 | SEQ ID NO: 6716 |
| rs1052486 | SEQ ID NO: 1985 | SEQ ID NO: 4351 | SEQ ID NO: 6717 |
| rs1150736 | SEQ ID NO: 1986 | SEQ ID NO: 4352 | SEQ ID NO: 6718 |
| rs130076 | SEQ ID NO: 1987 | SEQ ID NO: 4353 | SEQ ID NO: 6719 |
| rs2395174 | SEQ ID NO: 1988 | SEQ ID NO: 4354 | SEQ ID NO: 6720 |
| rs2252223 | SEQ ID NO: 1989 | SEQ ID NO: 4355 | SEQ ID NO: 6721 |
| rs1264303 | SEQ ID NO: 1990 | SEQ ID NO: 4356 | SEQ ID NO: 6722 |
| rs2240061 | SEQ ID NO: 1991 | SEQ ID NO: 4357 | SEQ ID NO: 6723 |
| rs1345229 | SEQ ID NO: 1992 | SEQ ID NO: 4358 | SEQ ID NO: 6724 |
| rs2395185 | SEQ ID NO: 1993 | SEQ ID NO: 4359 | SEQ ID NO: 6725 |
| rs3130283 | SEQ ID NO: 1994 | SEQ ID NO: 4360 | SEQ ID NO: 6726 |
| rs2257914 | SEQ ID NO: 1995 | SEQ ID NO: 4361 | SEQ ID NO: 6727 |
| rs916920 | SEQ ID NO: 1996 | SEQ ID NO: 4362 | SEQ ID NO: 6728 |
| rs2249059 | SEQ ID NO: 1997 | SEQ ID NO: 4363 | SEQ ID NO: 6729 |
| rs2267640 | SEQ ID NO: 1998 | SEQ ID NO: 4364 | SEQ ID NO: 6730 |
| rs1150765 | SEQ ID NO: 1999 | SEQ ID NO: 4365 | SEQ ID NO: 6731 |
| rs3749967 | SEQ ID NO: 2000 | SEQ ID NO: 4366 | SEQ ID NO: 6732 |
| rs3817963 | SEQ ID NO: 2001 | SEQ ID NO: 4367 | SEQ ID NO: 6733 |
| rs2051549 | SEQ ID NO: 2002 | SEQ ID NO: 4368 | SEQ ID NO: 6734 |
| rs1894408 | SEQ ID NO: 2003 | SEQ ID NO: 4369 | SEQ ID NO: 6735 |
| rs3130315 | SEQ ID NO: 2004 | SEQ ID NO: 4370 | SEQ ID NO: 6736 |
| rs761187 | SEQ ID NO: 2005 | SEQ ID NO: 4371 | SEQ ID NO: 6737 |
| rs3749966 | SEQ ID NO: 2006 | SEQ ID NO: 4372 | SEQ ID NO: 6738 |
| rs2395153 | SEQ ID NO: 2007 | SEQ ID NO: 4373 | SEQ ID NO: 6739 |
| rs1150735 | SEQ ID NO: 2008 | SEQ ID NO: 4374 | SEQ ID NO: 6740 |
| rs7843 | SEQ ID NO: 2009 | SEQ ID NO: 4375 | SEQ ID NO: 6741 |
| rs2074478 | SEQ ID NO: 2010 | SEQ ID NO: 4376 | SEQ ID NO: 6742 |
| rs204993 | SEQ ID NO: 2011 | SEQ ID NO: 4377 | SEQ ID NO: 6743 |
| rs2301271 | SEQ ID NO: 2012 | SEQ ID NO: 4378 | SEQ ID NO: 6744 |
| rs2284168 | SEQ ID NO: 2013 | SEQ ID NO: 4379 | SEQ ID NO: 6745 |
| rs11244 | SEQ ID NO: 2014 | SEQ ID NO: 4380 | SEQ ID NO: 6746 |
| rs1264583 | SEQ ID NO: 2015 | SEQ ID NO: 4381 | SEQ ID NO: 6747 |
| rs2074474 | SEQ ID NO: 2016 | SEQ ID NO: 4382 | SEQ ID NO: 6748 |
| rs2213571 | SEQ ID NO: 2017 | SEQ ID NO: 4383 | SEQ ID NO: 6749 |
| rs204991 | SEQ ID NO: 2018 | SEQ ID NO: 4384 | SEQ ID NO: 6750 |
| rs3129881 | SEQ ID NO: 2019 | SEQ ID NO: 4385 | SEQ ID NO: 6751 |
| rs1049623 | SEQ ID NO: 2020 | SEQ ID NO: 4386 | SEQ ID NO: 6752 |
| rs974962 | SEQ ID NO: 2021 | SEQ ID NO: 4387 | SEQ ID NO: 6753 |
| rs8512 | SEQ ID NO: 2022 | SEQ ID NO: 4388 | SEQ ID NO: 6754 |
| rs2071287 | SEQ ID NO: 2023 | SEQ ID NO: 4389 | SEQ ID NO: 6755 |
| rs1058318 | SEQ ID NO: 2024 | SEQ ID NO: 4390 | SEQ ID NO: 6756 |
| rs2269702 | SEQ ID NO: 2025 | SEQ ID NO: 4391 | SEQ ID NO: 6757 |
| rs2286404 | SEQ ID NO: 2026 | SEQ ID NO: 4392 | SEQ ID NO: 6758 |
| rs2269707 | SEQ ID NO: 2027 | SEQ ID NO: 4393 | SEQ ID NO: 6759 |
| rs2269424 | SEQ ID NO: 2028 | SEQ ID NO: 4394 | SEQ ID NO: 6760 |
| rs2040409 | SEQ ID NO: 2029 | SEQ ID NO: 4395 | SEQ ID NO: 6761 |
| rs241424 | SEQ ID NO: 2030 | SEQ ID NO: 4396 | SEQ ID NO: 6762 |
| rs2854275 | SEQ ID NO: 2031 | SEQ ID NO: 4397 | SEQ ID NO: 6763 |
| rs3880457 | SEQ ID NO: 2032 | SEQ ID NO: 4398 | SEQ ID NO: 6764 |
| rs4711206 | SEQ ID NO: 2033 | SEQ ID NO: 4399 | SEQ ID NO: 6765 |
| rs1233495 | SEQ ID NO: 2034 | SEQ ID NO: 4400 | SEQ ID NO: 6766 |
| rs3130055 | SEQ ID NO: 2035 | SEQ ID NO: 4401 | SEQ ID NO: 6767 |
| rs2071593 | SEQ ID NO: 2036 | SEQ ID NO: 4402 | SEQ ID NO: 6768 |
| rs7758397 | SEQ ID NO: 2037 | SEQ ID NO: 4403 | SEQ ID NO: 6769 |
| rs3130453 | SEQ ID NO: 2038 | SEQ ID NO: 4404 | SEQ ID NO: 6770 |
| rs2242657 | SEQ ID NO: 2039 | SEQ ID NO: 4405 | SEQ ID NO: 6771 |
| rs3130605 | SEQ ID NO: 2040 | SEQ ID NO: 4406 | SEQ ID NO: 6772 |
| rs3093559 | SEQ ID NO: 2041 | SEQ ID NO: 4407 | SEQ ID NO: 6773 |
| rs2235498 | SEQ ID NO: 2042 | SEQ ID NO: 4408 | SEQ ID NO: 6774 |
| rs3135034 | SEQ ID NO: 2043 | SEQ ID NO: 4409 | SEQ ID NO: 6775 |
| rs3129952 | SEQ ID NO: 2044 | SEQ ID NO: 4410 | SEQ ID NO: 6776 |
| rs6457602 | SEQ ID NO: 2045 | SEQ ID NO: 4411 | SEQ ID NO: 6777 |
| rs2523451 | SEQ ID NO: 2046 | SEQ ID NO: 4412 | SEQ ID NO: 6778 |
| rs497239 | SEQ ID NO: 2047 | SEQ ID NO: 4413 | SEQ ID NO: 6779 |
| rs4959050 | SEQ ID NO: 2048 | SEQ ID NO: 4414 | SEQ ID NO: 6780 |
| rs714289 | SEQ ID NO: 2049 | SEQ ID NO: 4415 | SEQ ID NO: 6781 |
| rs447250 | SEQ ID NO: 2050 | SEQ ID NO: 4416 | SEQ ID NO: 6782 |
| rs2844775 | SEQ ID NO: 2051 | SEQ ID NO: 4417 | SEQ ID NO: 6783 |
| rs3135002 | SEQ ID NO: 2052 | SEQ ID NO: 4418 | SEQ ID NO: 6784 |
| rs3077 | SEQ ID NO: 2053 | SEQ ID NO: 4419 | SEQ ID NO: 6785 |
| rs3094205 | SEQ ID NO: 2054 | SEQ ID NO: 4420 | SEQ ID NO: 6786 |
| rs6927088 | SEQ ID NO: 2055 | SEQ ID NO: 4421 | SEQ ID NO: 6787 |
| rs2857174 | SEQ ID NO: 2056 | SEQ ID NO: 4422 | SEQ ID NO: 6788 |
| rs181997 | SEQ ID NO: 2057 | SEQ ID NO: 4423 | SEQ ID NO: 6789 |
| rs261950 | SEQ ID NO: 2058 | SEQ ID NO: 4424 | SEQ ID NO: 6790 |
| rs3117329 | SEQ ID NO: 2059 | SEQ ID NO: 4425 | SEQ ID NO: 6791 |
| rs3128931 | SEQ ID NO: 2060 | SEQ ID NO: 4426 | SEQ ID NO: 6792 |
| rs6904456 | SEQ ID NO: 2061 | SEQ ID NO: 4427 | SEQ ID NO: 6793 |
| rs4713216 | SEQ ID NO: 2062 | SEQ ID NO: 4428 | SEQ ID NO: 6794 |
| rs3097671 | SEQ ID NO: 2063 | SEQ ID NO: 4429 | SEQ ID NO: 6795 |
| rs3135368 | SEQ ID NO: 2064 | SEQ ID NO: 4430 | SEQ ID NO: 6796 |
| rs1264323 | SEQ ID NO: 2065 | SEQ ID NO: 4431 | SEQ ID NO: 6797 |
| rs3094073 | SEQ ID NO: 2066 | SEQ ID NO: 4432 | SEQ ID NO: 6798 |
| rs2523995 | SEQ ID NO: 2067 | SEQ ID NO: 4433 | SEQ ID NO: 6799 |
| rs3131635 | SEQ ID NO: 2068 | SEQ ID NO: 4434 | SEQ ID NO: 6800 |
| rs1610724 | SEQ ID NO: 2069 | SEQ ID NO: 4435 | SEQ ID NO: 6801 |
| rs4713337 | SEQ ID NO: 2070 | SEQ ID NO: 4436 | SEQ ID NO: 6802 |
| rs2855442 | SEQ ID NO: 2071 | SEQ ID NO: 4437 | SEQ ID NO: 6803 |
| rs885915 | SEQ ID NO: 2072 | SEQ ID NO: 4438 | SEQ ID NO: 6804 |
| rs241440 | SEQ ID NO: 2073 | SEQ ID NO: 4439 | SEQ ID NO: 6805 |
| rs879882 | SEQ ID NO: 2074 | SEQ ID NO: 4440 | SEQ ID NO: 6806 |
| rs6937061 | SEQ ID NO: 2075 | SEQ ID NO: 4441 | SEQ ID NO: 6807 |
| rs2023372 | SEQ ID NO: 2076 | SEQ ID NO: 4442 | SEQ ID NO: 6808 |
| rs1610601 | SEQ ID NO: 2077 | SEQ ID NO: 4443 | SEQ ID NO: 6809 |
| rs29228 | SEQ ID NO: 2078 | SEQ ID NO: 4444 | SEQ ID NO: 6810 |
| rs7454372 | SEQ ID NO: 2079 | SEQ ID NO: 4445 | SEQ ID NO: 6811 |
| rs707937 | SEQ ID NO: 2080 | SEQ ID NO: 4446 | SEQ ID NO: 6812 |
| rs7341251 | SEQ ID NO: 2081 | SEQ ID NO: 4447 | SEQ ID NO: 6813 |
| rs1800750 | SEQ ID NO: 2082 | SEQ ID NO: 4448 | SEQ ID NO: 6814 |
| rs2394518 | SEQ ID NO: 2083 | SEQ ID NO: 4449 | SEQ ID NO: 6815 |
| rs2301225 | SEQ ID NO: 2084 | SEQ ID NO: 4450 | SEQ ID NO: 6816 |
| rs1264459 | SEQ ID NO: 2085 | SEQ ID NO: 4451 | SEQ ID NO: 6817 |
| rs7773955 | SEQ ID NO: 2086 | SEQ ID NO: 4452 | SEQ ID NO: 6818 |
| rs15912 | SEQ ID NO: 2087 | SEQ ID NO: 4453 | SEQ ID NO: 6819 |
| rs1419675 | SEQ ID NO: 2088 | SEQ ID NO: 4454 | SEQ ID NO: 6820 |
| rs7450074 | SEQ ID NO: 2089 | SEQ ID NO: 4455 | SEQ ID NO: 6821 |
| rs437179 | SEQ ID NO: 2090 | SEQ ID NO: 4456 | SEQ ID NO: 6822 |
| rs3132486 | SEQ ID NO: 2091 | SEQ ID NO: 4457 | SEQ ID NO: 6823 |
| rs2071286 | SEQ ID NO: 2092 | SEQ ID NO: 4458 | SEQ ID NO: 6824 |
| rs2395029 | SEQ ID NO: 2093 | SEQ ID NO: 4459 | SEQ ID NO: 6825 |
| rs7765791 | SEQ ID NO: 2094 | SEQ ID NO: 4460 | SEQ ID NO: 6826 |
| rs3094576 | SEQ ID NO: 2095 | SEQ ID NO: 4461 | SEQ ID NO: 6827 |
| rs3819269 | SEQ ID NO: 2096 | SEQ ID NO: 4462 | SEQ ID NO: 6828 |
| rs2074471 | SEQ ID NO: 2097 | SEQ ID NO: 4463 | SEQ ID NO: 6829 |
| rs6929716 | SEQ ID NO: 2098 | SEQ ID NO: 4464 | SEQ ID NO: 6830 |
| rs2523608 | SEQ ID NO: 2099 | SEQ ID NO: 4465 | SEQ ID NO: 6831 |
| rs1264540 | SEQ ID NO: 2100 | SEQ ID NO: 4466 | SEQ ID NO: 6832 |
| rs3116996 | SEQ ID NO: 2101 | SEQ ID NO: 4467 | SEQ ID NO: 6833 |
| rs3130933 | SEQ ID NO: 2102 | SEQ ID NO: 4468 | SEQ ID NO: 6834 |
| rs6940867 | SEQ ID NO: 2103 | SEQ ID NO: 4469 | SEQ ID NO: 6835 |
| rs2535250 | SEQ ID NO: 2104 | SEQ ID NO: 4470 | SEQ ID NO: 6836 |
| rs6903608 | SEQ ID NO: 2105 | SEQ ID NO: 4471 | SEQ ID NO: 6837 |
| rs3117435 | SEQ ID NO: 2106 | SEQ ID NO: 4472 | SEQ ID NO: 6838 |
| rs1264425 | SEQ ID NO: 2107 | SEQ ID NO: 4473 | SEQ ID NO: 6839 |
| rs3873342 | SEQ ID NO: 2108 | SEQ ID NO: 4474 | SEQ ID NO: 6840 |
| rs1265059 | SEQ ID NO: 2109 | SEQ ID NO: 4475 | SEQ ID NO: 6841 |
| rs6911634 | SEQ ID NO: 2110 | SEQ ID NO: 4476 | SEQ ID NO: 6842 |
| rs464921 | SEQ ID NO: 2111 | SEQ ID NO: 4477 | SEQ ID NO: 6843 |
| rs4713221 | SEQ ID NO: 2112 | SEQ ID NO: 4478 | SEQ ID NO: 6844 |
| rs362544 | SEQ ID NO: 2113 | SEQ ID NO: 4479 | SEQ ID NO: 6845 |
| rs3130977 | SEQ ID NO: 2114 | SEQ ID NO: 4480 | SEQ ID NO: 6846 |
| rs2395352 | SEQ ID NO: 2115 | SEQ ID NO: 4481 | SEQ ID NO: 6847 |
| rs984802 | SEQ ID NO: 2116 | SEQ ID NO: 4482 | SEQ ID NO: 6848 |
| rs1049633 | SEQ ID NO: 2117 | SEQ ID NO: 4483 | SEQ ID NO: 6849 |
| rs6923005 | SEQ ID NO: 2118 | SEQ ID NO: 4484 | SEQ ID NO: 6850 |
| rs2001181 | SEQ ID NO: 2119 | SEQ ID NO: 4485 | SEQ ID NO: 6851 |
| rs2071541 | SEQ ID NO: 2120 | SEQ ID NO: 4486 | SEQ ID NO: 6852 |
| rs3807039 | SEQ ID NO: 2121 | SEQ ID NO: 4487 | SEQ ID NO: 6853 |
| rs6918223 | SEQ ID NO: 2122 | SEQ ID NO: 4488 | SEQ ID NO: 6854 |
| rs3130501 | SEQ ID NO: 2123 | SEQ ID NO: 4489 | SEQ ID NO: 6855 |
| rs3869092 | SEQ ID NO: 2124 | SEQ ID NO: 4490 | SEQ ID NO: 6856 |
| rs1736920 | SEQ ID NO: 2125 | SEQ ID NO: 4491 | SEQ ID NO: 6857 |
| rs3823418 | SEQ ID NO: 2126 | SEQ ID NO: 4492 | SEQ ID NO: 6858 |
| rs2844802 | SEQ ID NO: 2127 | SEQ ID NO: 4493 | SEQ ID NO: 6859 |
| rs2394412 | SEQ ID NO: 2128 | SEQ ID NO: 4494 | SEQ ID NO: 6860 |
| rs439844 | SEQ ID NO: 2129 | SEQ ID NO: 4495 | SEQ ID NO: 6861 |
| rs3763309 | SEQ ID NO: 2130 | SEQ ID NO: 4496 | SEQ ID NO: 6862 |
| rs7383312 | SEQ ID NO: 2131 | SEQ ID NO: 4497 | SEQ ID NO: 6863 |
| rs1633021 | SEQ ID NO: 2132 | SEQ ID NO: 4498 | SEQ ID NO: 6864 |
| rs1367727 | SEQ ID NO: 2133 | SEQ ID NO: 4499 | SEQ ID NO: 6865 |
| rs2269423 | SEQ ID NO: 2134 | SEQ ID NO: 4500 | SEQ ID NO: 6866 |
| rs29269 | SEQ ID NO: 2135 | SEQ ID NO: 4501 | SEQ ID NO: 6867 |
| rs2894054 | SEQ ID NO: 2136 | SEQ ID NO: 4502 | SEQ ID NO: 6868 |
| rs2534674 | SEQ ID NO: 2137 | SEQ ID NO: 4503 | SEQ ID NO: 6869 |
| rs6915177 | SEQ ID NO: 2138 | SEQ ID NO: 4504 | SEQ ID NO: 6870 |
| rs4713559 | SEQ ID NO: 2139 | SEQ ID NO: 4505 | SEQ ID NO: 6871 |
| rs7382836 | SEQ ID NO: 2140 | SEQ ID NO: 4506 | SEQ ID NO: 6872 |
| rs4071986 | SEQ ID NO: 2141 | SEQ ID NO: 4507 | SEQ ID NO: 6873 |
| rs735883 | SEQ ID NO: 2142 | SEQ ID NO: 4508 | SEQ ID NO: 6874 |
| rs3793127 | SEQ ID NO: 2143 | SEQ ID NO: 4509 | SEQ ID NO: 6875 |
| rs2301269 | SEQ ID NO: 2144 | SEQ ID NO: 4510 | SEQ ID NO: 6876 |
| rs2844685 | SEQ ID NO: 2145 | SEQ ID NO: 4511 | SEQ ID NO: 6877 |
| rs6908943 | SEQ ID NO: 2146 | SEQ ID NO: 4512 | SEQ ID NO: 6878 |
| rs7383578 | SEQ ID NO: 2147 | SEQ ID NO: 4513 | SEQ ID NO: 6879 |
| rs1633088 | SEQ ID NO: 2148 | SEQ ID NO: 4514 | SEQ ID NO: 6880 |
| rs3871247 | SEQ ID NO: 2149 | SEQ ID NO: 4515 | SEQ ID NO: 6881 |
| rs461338 | SEQ ID NO: 2150 | SEQ ID NO: 4516 | SEQ ID NO: 6882 |
| rs3807031 | SEQ ID NO: 2151 | SEQ ID NO: 4517 | SEQ ID NO: 6883 |
| rs6911709 | SEQ ID NO: 2152 | SEQ ID NO: 4518 | SEQ ID NO: 6884 |
| rs2524005 | SEQ ID NO: 2153 | SEQ ID NO: 4519 | SEQ ID NO: 6885 |
| rs1736991 | SEQ ID NO: 2154 | SEQ ID NO: 4520 | SEQ ID NO: 6886 |
| rs4713422 | SEQ ID NO: 2155 | SEQ ID NO: 4521 | SEQ ID NO: 6887 |
| rs6921921 | SEQ ID NO: 2156 | SEQ ID NO: 4522 | SEQ ID NO: 6888 |
| rs7453137 | SEQ ID NO: 2157 | SEQ ID NO: 4523 | SEQ ID NO: 6889 |
| rs1801823 | SEQ ID NO: 2158 | SEQ ID NO: 4524 | SEQ ID NO: 6890 |
| rs498240 | SEQ ID NO: 2159 | SEQ ID NO: 4525 | SEQ ID NO: 6891 |
| rs6906730 | SEQ ID NO: 2160 | SEQ ID NO: 4526 | SEQ ID NO: 6892 |
| rs2057726 | SEQ ID NO: 2161 | SEQ ID NO: 4527 | SEQ ID NO: 6893 |
| rs2523989 | SEQ ID NO: 2162 | SEQ ID NO: 4528 | SEQ ID NO: 6894 |
| rs2071788 | SEQ ID NO: 2163 | SEQ ID NO: 4529 | SEQ ID NO: 6895 |
| rs4713214 | SEQ ID NO: 2164 | SEQ ID NO: 4530 | SEQ ID NO: 6896 |
| rs29273 | SEQ ID NO: 2165 | SEQ ID NO: 4531 | SEQ ID NO: 6897 |
| rs2517912 | SEQ ID NO: 2166 | SEQ ID NO: 4532 | SEQ ID NO: 6898 |
| rs1265156 | SEQ ID NO: 2167 | SEQ ID NO: 4533 | SEQ ID NO: 6899 |
| rs4386816 | SEQ ID NO: 2168 | SEQ ID NO: 4534 | SEQ ID NO: 6900 |
| rs2075702 | SEQ ID NO: 2169 | SEQ ID NO: 4535 | SEQ ID NO: 6901 |
| rs1264616 | SEQ ID NO: 2170 | SEQ ID NO: 4536 | SEQ ID NO: 6902 |
| rs1419633 | SEQ ID NO: 2171 | SEQ ID NO: 4537 | SEQ ID NO: 6903 |
| rs389883 | SEQ ID NO: 2172 | SEQ ID NO: 4538 | SEQ ID NO: 6904 |
| rs2523921 | SEQ ID NO: 2173 | SEQ ID NO: 4539 | SEQ ID NO: 6905 |
| rs209474 | SEQ ID NO: 2174 | SEQ ID NO: 4540 | SEQ ID NO: 6906 |
| rs2532921 | SEQ ID NO: 2175 | SEQ ID NO: 4541 | SEQ ID NO: 6907 |
| rs2207338 | SEQ ID NO: 2176 | SEQ ID NO: 4542 | SEQ ID NO: 6908 |
| rs2844829 | SEQ ID NO: 2177 | SEQ ID NO: 4543 | SEQ ID NO: 6909 |
| rs1035798 | SEQ ID NO: 2178 | SEQ ID NO: 4544 | SEQ ID NO: 6910 |
| rs2857597 | SEQ ID NO: 2179 | SEQ ID NO: 4545 | SEQ ID NO: 6911 |
| rs2747421 | SEQ ID NO: 2180 | SEQ ID NO: 4546 | SEQ ID NO: 6912 |
| rs2246618 | SEQ ID NO: 2181 | SEQ ID NO: 4547 | SEQ ID NO: 6913 |
| rs2855812 | SEQ ID NO: 2182 | SEQ ID NO: 4548 | SEQ ID NO: 6914 |
| rs6457259 | SEQ ID NO: 2183 | SEQ ID NO: 4549 | SEQ ID NO: 6915 |
| rs2071799 | SEQ ID NO: 2184 | SEQ ID NO: 4550 | SEQ ID NO: 6916 |
| rs7754054 | SEQ ID NO: 2185 | SEQ ID NO: 4551 | SEQ ID NO: 6917 |
| rs1049526 | SEQ ID NO: 2186 | SEQ ID NO: 4552 | SEQ ID NO: 6918 |
| rs2284191 | SEQ ID NO: 2187 | SEQ ID NO: 4553 | SEQ ID NO: 6919 |
| rs1655944 | SEQ ID NO: 2188 | SEQ ID NO: 4554 | SEQ ID NO: 6920 |
| rs7451258 | SEQ ID NO: 2189 | SEQ ID NO: 4555 | SEQ ID NO: 6921 |
| rs2844665 | SEQ ID NO: 2190 | SEQ ID NO: 4556 | SEQ ID NO: 6922 |
| rs3763364 | SEQ ID NO: 2191 | SEQ ID NO: 4557 | SEQ ID NO: 6923 |
| rs3128982 | SEQ ID NO: 2192 | SEQ ID NO: 4558 | SEQ ID NO: 6924 |
| rs6457358 | SEQ ID NO: 2193 | SEQ ID NO: 4559 | SEQ ID NO: 6925 |
| rs7758512 | SEQ ID NO: 2194 | SEQ ID NO: 4560 | SEQ ID NO: 6926 |
| rs3115673 | SEQ ID NO: 2195 | SEQ ID NO: 4561 | SEQ ID NO: 6927 |
| rs7029 | SEQ ID NO: 2196 | SEQ ID NO: 4562 | SEQ ID NO: 6928 |
| rs1041885 | SEQ ID NO: 2197 | SEQ ID NO: 4563 | SEQ ID NO: 6929 |
| rs259939 | SEQ ID NO: 2198 | SEQ ID NO: 4564 | SEQ ID NO: 6930 |
| rs3093993 | SEQ ID NO: 2199 | SEQ ID NO: 4565 | SEQ ID NO: 6931 |
| rs6457595 | SEQ ID NO: 2200 | SEQ ID NO: 4566 | SEQ ID NO: 6932 |
| rs3128844 | SEQ ID NO: 2201 | SEQ ID NO: 4567 | SEQ ID NO: 6933 |
| rs3094212 | SEQ ID NO: 2202 | SEQ ID NO: 4568 | SEQ ID NO: 6934 |
| rs1264695 | SEQ ID NO: 2203 | SEQ ID NO: 4569 | SEQ ID NO: 6935 |
| rs2516393 | SEQ ID NO: 2204 | SEQ ID NO: 4570 | SEQ ID NO: 6936 |
| rs209146 | SEQ ID NO: 2205 | SEQ ID NO: 4571 | SEQ ID NO: 6937 |
| rs4713243 | SEQ ID NO: 2206 | SEQ ID NO: 4572 | SEQ ID NO: 6938 |
| rs532385 | SEQ ID NO: 2207 | SEQ ID NO: 4573 | SEQ ID NO: 6939 |
| rs1610628 | SEQ ID NO: 2208 | SEQ ID NO: 4574 | SEQ ID NO: 6940 |
| rs8111 | SEQ ID NO: 2209 | SEQ ID NO: 4575 | SEQ ID NO: 6941 |
| rs2242660 | SEQ ID NO: 2210 | SEQ ID NO: 4576 | SEQ ID NO: 6942 |
| rs3115592 | SEQ ID NO: 2211 | SEQ ID NO: 4577 | SEQ ID NO: 6943 |
| rs3135029 | SEQ ID NO: 2212 | SEQ ID NO: 4578 | SEQ ID NO: 6944 |
| rs3869109 | SEQ ID NO: 2213 | SEQ ID NO: 4579 | SEQ ID NO: 6945 |
| rs2066951 | SEQ ID NO: 2214 | SEQ ID NO: 4580 | SEQ ID NO: 6946 |
| rs2282851 | SEQ ID NO: 2215 | SEQ ID NO: 4581 | SEQ ID NO: 6947 |
| rs2517713 | SEQ ID NO: 2216 | SEQ ID NO: 4582 | SEQ ID NO: 6948 |
| rs7770505 | SEQ ID NO: 2217 | SEQ ID NO: 4583 | SEQ ID NO: 6949 |
| rs3117294 | SEQ ID NO: 2218 | SEQ ID NO: 4584 | SEQ ID NO: 6950 |
| rs2857218 | SEQ ID NO: 2219 | SEQ ID NO: 4585 | SEQ ID NO: 6951 |
| rs2242656 | SEQ ID NO: 2220 | SEQ ID NO: 4586 | SEQ ID NO: 6952 |
| rs2275854 | SEQ ID NO: 2221 | SEQ ID NO: 4587 | SEQ ID NO: 6953 |
| rs3130050 | SEQ ID NO: 2222 | SEQ ID NO: 4588 | SEQ ID NO: 6954 |
| rs7745413 | SEQ ID NO: 2223 | SEQ ID NO: 4589 | SEQ ID NO: 6955 |
| rs2894066 | SEQ ID NO: 2224 | SEQ ID NO: 4590 | SEQ ID NO: 6956 |
| rs3213469 | SEQ ID NO: 2225 | SEQ ID NO: 4591 | SEQ ID NO: 6957 |
| rs406936 | SEQ ID NO: 2226 | SEQ ID NO: 4592 | SEQ ID NO: 6958 |
| rs3916765 | SEQ ID NO: 2227 | SEQ ID NO: 4593 | SEQ ID NO: 6959 |
| rs3873257 | SEQ ID NO: 2228 | SEQ ID NO: 4594 | SEQ ID NO: 6960 |
| rs453658 | SEQ ID NO: 2229 | SEQ ID NO: 4595 | SEQ ID NO: 6961 |
| rs660594 | SEQ ID NO: 2230 | SEQ ID NO: 4596 | SEQ ID NO: 6962 |
| rs1008403 | SEQ ID NO: 2231 | SEQ ID NO: 4597 | SEQ ID NO: 6963 |
| rs763009 | SEQ ID NO: 2232 | SEQ ID NO: 4598 | SEQ ID NO: 6964 |
| rs3130649 | SEQ ID NO: 2233 | SEQ ID NO: 4599 | SEQ ID NO: 6965 |
| rs7752270 | SEQ ID NO: 2234 | SEQ ID NO: 4600 | SEQ ID NO: 6966 |
| rs659445 | SEQ ID NO: 2235 | SEQ ID NO: 4601 | SEQ ID NO: 6967 |
| rs7753474 | SEQ ID NO: 2236 | SEQ ID NO: 4602 | SEQ ID NO: 6968 |
| rs3130762 | SEQ ID NO: 2237 | SEQ ID NO: 4603 | SEQ ID NO: 6969 |
| rs3094127 | SEQ ID NO: 2238 | SEQ ID NO: 4604 | SEQ ID NO: 6970 |
| rs7754598 | SEQ ID NO: 2239 | SEQ ID NO: 4605 | SEQ ID NO: 6971 |
| rs7774255 | SEQ ID NO: 2240 | SEQ ID NO: 4606 | SEQ ID NO: 6972 |
| rs465506 | SEQ ID NO: 2241 | SEQ ID NO: 4607 | SEQ ID NO: 6973 |
| rs2853961 | SEQ ID NO: 2242 | SEQ ID NO: 4608 | SEQ ID NO: 6974 |
| rs3815101 | SEQ ID NO: 2243 | SEQ ID NO: 4609 | SEQ ID NO: 6975 |
| rs2517892 | SEQ ID NO: 2244 | SEQ ID NO: 4610 | SEQ ID NO: 6976 |
| rs2187689 | SEQ ID NO: 2245 | SEQ ID NO: 4611 | SEQ ID NO: 6977 |
| rs6901092 | SEQ ID NO: 2246 | SEQ ID NO: 4612 | SEQ ID NO: 6978 |
| rs2857605 | SEQ ID NO: 2247 | SEQ ID NO: 4613 | SEQ ID NO: 6979 |
| rs2516676 | SEQ ID NO: 2248 | SEQ ID NO: 4614 | SEQ ID NO: 6980 |
| rs6922756 | SEQ ID NO: 2249 | SEQ ID NO: 4615 | SEQ ID NO: 6981 |
| rs2074475 | SEQ ID NO: 2250 | SEQ ID NO: 4616 | SEQ ID NO: 6982 |
| rs1264432 | SEQ ID NO: 2251 | SEQ ID NO: 4617 | SEQ ID NO: 6983 |
| rs2267633 | SEQ ID NO: 2252 | SEQ ID NO: 4618 | SEQ ID NO: 6984 |
| rs2517819 | SEQ ID NO: 2253 | SEQ ID NO: 4619 | SEQ ID NO: 6985 |
| rs3130637 | SEQ ID NO: 2254 | SEQ ID NO: 4620 | SEQ ID NO: 6986 |
| rs3116837 | SEQ ID NO: 2255 | SEQ ID NO: 4621 | SEQ ID NO: 6987 |
| rs3793125 | SEQ ID NO: 2256 | SEQ ID NO: 4622 | SEQ ID NO: 6988 |
| rs757257 | SEQ ID NO: 2257 | SEQ ID NO: 4623 | SEQ ID NO: 6989 |
| rs3117038 | SEQ ID NO: 2258 | SEQ ID NO: 4624 | SEQ ID NO: 6990 |
| rs2523589 | SEQ ID NO: 2259 | SEQ ID NO: 4625 | SEQ ID NO: 6991 |
| rs2747479 | SEQ ID NO: 2260 | SEQ ID NO: 4626 | SEQ ID NO: 6992 |
| rs3130244 | SEQ ID NO: 2261 | SEQ ID NO: 4627 | SEQ ID NO: 6993 |
| rs7357041 | SEQ ID NO: 2262 | SEQ ID NO: 4628 | SEQ ID NO: 6994 |
| rs4713504 | SEQ ID NO: 2263 | SEQ ID NO: 4629 | SEQ ID NO: 6995 |
| rs2187580 | SEQ ID NO: 2264 | SEQ ID NO: 4630 | SEQ ID NO: 6996 |
| rs3132945 | SEQ ID NO: 2265 | SEQ ID NO: 4631 | SEQ ID NO: 6997 |
| rs3998751 | SEQ ID NO: 2266 | SEQ ID NO: 4632 | SEQ ID NO: 6998 |
| rs2860580 | SEQ ID NO: 2267 | SEQ ID NO: 4633 | SEQ ID NO: 6999 |
| rs7757916 | SEQ ID NO: 2268 | SEQ ID NO: 4634 | SEQ ID NO: 7000 |
| rs3130014 | SEQ ID NO: 2269 | SEQ ID NO: 4635 | SEQ ID NO: 7001 |
| rs3830076 | SEQ ID NO: 2270 | SEQ ID NO: 4636 | SEQ ID NO: 7002 |
| rs2158280 | SEQ ID NO: 2271 | SEQ ID NO: 4637 | SEQ ID NO: 7003 |
| rs1233391 | SEQ ID NO: 2272 | SEQ ID NO: 4638 | SEQ ID NO: 7004 |
| rs2844477 | SEQ ID NO: 2273 | SEQ ID NO: 4639 | SEQ ID NO: 7005 |
| rs7453097 | SEQ ID NO: 2274 | SEQ ID NO: 4640 | SEQ ID NO: 7006 |
| rs3130816 | SEQ ID NO: 2275 | SEQ ID NO: 4641 | SEQ ID NO: 7007 |
| rs4151648 | SEQ ID NO: 2276 | SEQ ID NO: 4642 | SEQ ID NO: 7008 |
| rs3131090 | SEQ ID NO: 2277 | SEQ ID NO: 4643 | SEQ ID NO: 7009 |
| rs1063318 | SEQ ID NO: 2278 | SEQ ID NO: 4644 | SEQ ID NO: 7010 |
| rs3130660 | SEQ ID NO: 2279 | SEQ ID NO: 4645 | SEQ ID NO: 7011 |
| rs1264549 | SEQ ID NO: 2280 | SEQ ID NO: 4646 | SEQ ID NO: 7012 |
| rs2299851 | SEQ ID NO: 2281 | SEQ ID NO: 4647 | SEQ ID NO: 7013 |
| rs2844682 | SEQ ID NO: 2282 | SEQ ID NO: 4648 | SEQ ID NO: 7014 |
| rs2051625 | SEQ ID NO: 2283 | SEQ ID NO: 4649 | SEQ ID NO: 7015 |
| rs2178899 | SEQ ID NO: 2284 | SEQ ID NO: 4650 | SEQ ID NO: 7016 |
| rs3130389 | SEQ ID NO: 2285 | SEQ ID NO: 4651 | SEQ ID NO: 7017 |
| rs3749981 | SEQ ID NO: 2286 | SEQ ID NO: 4652 | SEQ ID NO: 7018 |
| rs1233386 | SEQ ID NO: 2287 | SEQ ID NO: 4653 | SEQ ID NO: 7019 |
| rs2142234 | SEQ ID NO: 2288 | SEQ ID NO: 4654 | SEQ ID NO: 7020 |
| rs2747419 | SEQ ID NO: 2289 | SEQ ID NO: 4655 | SEQ ID NO: 7021 |
| rs356971 | SEQ ID NO: 2290 | SEQ ID NO: 4656 | SEQ ID NO: 7022 |
| rs1079541 | SEQ ID NO: 2291 | SEQ ID NO: 4657 | SEQ ID NO: 7023 |
| rs2273192 | SEQ ID NO: 2292 | SEQ ID NO: 4658 | SEQ ID NO: 7024 |
| rs1015466 | SEQ ID NO: 2293 | SEQ ID NO: 4659 | SEQ ID NO: 7025 |
| rs4947324 | SEQ ID NO: 2294 | SEQ ID NO: 4660 | SEQ ID NO: 7026 |
| rs2071295 | SEQ ID NO: 2295 | SEQ ID NO: 4661 | SEQ ID NO: 7027 |
| rs3130010 | SEQ ID NO: 2296 | SEQ ID NO: 4662 | SEQ ID NO: 7028 |
| rs2517424 | SEQ ID NO: 2297 | SEQ ID NO: 4663 | SEQ ID NO: 7029 |
| rs3093986 | SEQ ID NO: 2298 | SEQ ID NO: 4664 | SEQ ID NO: 7030 |
| rs2006752 | SEQ ID NO: 2299 | SEQ ID NO: 4665 | SEQ ID NO: 7031 |
| rs2530699 | SEQ ID NO: 2300 | SEQ ID NO: 4666 | SEQ ID NO: 7032 |
| rs707917 | SEQ ID NO: 2301 | SEQ ID NO: 4667 | SEQ ID NO: 7033 |
| rs6531 | SEQ ID NO: 2302 | SEQ ID NO: 4668 | SEQ ID NO: 7034 |
| rs2206042 | SEQ ID NO: 2303 | SEQ ID NO: 4669 | SEQ ID NO: 7035 |
| rs6457452 | SEQ ID NO: 2304 | SEQ ID NO: 4670 | SEQ ID NO: 7036 |
| rs1362064 | SEQ ID NO: 2305 | SEQ ID NO: 4671 | SEQ ID NO: 7037 |
| rs4424066 | SEQ ID NO: 2306 | SEQ ID NO: 4672 | SEQ ID NO: 7038 |
| rs3094110 | SEQ ID NO: 2307 | SEQ ID NO: 4673 | SEQ ID NO: 7039 |
| rs2242668 | SEQ ID NO: 2308 | SEQ ID NO: 4674 | SEQ ID NO: 7040 |
| rs6910451 | SEQ ID NO: 2309 | SEQ ID NO: 4675 | SEQ ID NO: 7041 |
| rs3823355 | SEQ ID NO: 2310 | SEQ ID NO: 4676 | SEQ ID NO: 7042 |
| rs3130667 | SEQ ID NO: 2311 | SEQ ID NO: 4677 | SEQ ID NO: 7043 |
| rs2071293 | SEQ ID NO: 2312 | SEQ ID NO: 4678 | SEQ ID NO: 7044 |
| rs1063478 | SEQ ID NO: 2313 | SEQ ID NO: 4679 | SEQ ID NO: 7045 |
| rs2242654 | SEQ ID NO: 2314 | SEQ ID NO: 4680 | SEQ ID NO: 7046 |
| rs2394994 | SEQ ID NO: 2315 | SEQ ID NO: 4681 | SEQ ID NO: 7047 |
| rs3130805 | SEQ ID NO: 2316 | SEQ ID NO: 4682 | SEQ ID NO: 7048 |
| rs2269429 | SEQ ID NO: 2317 | SEQ ID NO: 4683 | SEQ ID NO: 7049 |
| rs3132587 | SEQ ID NO: 2318 | SEQ ID NO: 4684 | SEQ ID NO: 7050 |
| rs2300825 | SEQ ID NO: 2319 | SEQ ID NO: 4685 | SEQ ID NO: 7051 |
| rs7744304 | SEQ ID NO: 2320 | SEQ ID NO: 4686 | SEQ ID NO: 7052 |
| rs1233388 | SEQ ID NO: 2321 | SEQ ID NO: 4687 | SEQ ID NO: 7053 |
| rs4711279 | SEQ ID NO: 2322 | SEQ ID NO: 4688 | SEQ ID NO: 7054 |
| rs6915800 | SEQ ID NO: 2323 | SEQ ID NO: 4689 | SEQ ID NO: 7055 |
| rs11908 | SEQ ID NO: 2324 | SEQ ID NO: 4690 | SEQ ID NO: 7056 |
| rs1059288 | SEQ ID NO: 2325 | SEQ ID NO: 4691 | SEQ ID NO: 7057 |
| rs3025657 | SEQ ID NO: 2326 | SEQ ID NO: 4692 | SEQ ID NO: 7058 |
| rs213209 | SEQ ID NO: 2327 | SEQ ID NO: 4693 | SEQ ID NO: 7059 |
| rs7766900 | SEQ ID NO: 2328 | SEQ ID NO: 4694 | SEQ ID NO: 7060 |
| rs3130268 | SEQ ID NO: 2329 | SEQ ID NO: 4695 | SEQ ID NO: 7061 |
| rs3749952 | SEQ ID NO: 2330 | SEQ ID NO: 4696 | SEQ ID NO: 7062 |
| rs6456951 | SEQ ID NO: 2331 | SEQ ID NO: 4697 | SEQ ID NO: 7063 |
| rs2523974 | SEQ ID NO: 2332 | SEQ ID NO: 4698 | SEQ ID NO: 7064 |
| rs6904236 | SEQ ID NO: 2333 | SEQ ID NO: 4699 | SEQ ID NO: 7065 |
| rs4713518 | SEQ ID NO: 2334 | SEQ ID NO: 4700 | SEQ ID NO: 7066 |
| rs2267646 | SEQ ID NO: 2335 | SEQ ID NO: 4701 | SEQ ID NO: 7067 |
| rs502626 | SEQ ID NO: 2336 | SEQ ID NO: 4702 | SEQ ID NO: 7068 |
| rs6468 | SEQ ID NO: 2337 | SEQ ID NO: 4703 | SEQ ID NO: 7069 |
| rs3129110 | SEQ ID NO: 2338 | SEQ ID NO: 4704 | SEQ ID NO: 7070 |
| rs6456947 | SEQ ID NO: 2339 | SEQ ID NO: 4705 | SEQ ID NO: 7071 |
| rs3129154 | SEQ ID NO: 2340 | SEQ ID NO: 4706 | SEQ ID NO: 7072 |
| rs1009382 | SEQ ID NO: 2341 | SEQ ID NO: 4707 | SEQ ID NO: 7073 |
| rs1043618 | SEQ ID NO: 2342 | SEQ ID NO: 4708 | SEQ ID NO: 7074 |
| rs7754402 | SEQ ID NO: 2343 | SEQ ID NO: 4709 | SEQ ID NO: 7075 |
| rs2269706 | SEQ ID NO: 2344 | SEQ ID NO: 4710 | SEQ ID NO: 7076 |
| rs4713505 | SEQ ID NO: 2345 | SEQ ID NO: 4711 | SEQ ID NO: 7077 |
| rs205000 | SEQ ID NO: 2346 | SEQ ID NO: 4712 | SEQ ID NO: 7078 |
| rs2071278 | SEQ ID NO: 2347 | SEQ ID NO: 4713 | SEQ ID NO: 7079 |
| rs477005 | SEQ ID NO: 2348 | SEQ ID NO: 4714 | SEQ ID NO: 7080 |
| rs535586 | SEQ ID NO: 2349 | SEQ ID NO: 4715 | SEQ ID NO: 7081 |
| rs1052248 | SEQ ID NO: 2350 | SEQ ID NO: 4716 | SEQ ID NO: 7082 |
| rs621701 | SEQ ID NO: 2351 | SEQ ID NO: 4717 | SEQ ID NO: 7083 |
| rs2072679 | SEQ ID NO: 2352 | SEQ ID NO: 4718 | SEQ ID NO: 7084 |
| rs805257 | SEQ ID NO: 2353 | SEQ ID NO: 4719 | SEQ ID NO: 7085 |
| rs469064 | SEQ ID NO: 2354 | SEQ ID NO: 4720 | SEQ ID NO: 7086 |
| rs1264517 | SEQ ID NO: 2355 | SEQ ID NO: 4721 | SEQ ID NO: 7087 |
| rs211457 | SEQ ID NO: 2356 | SEQ ID NO: 4722 | SEQ ID NO: 7088 |
| rs537160 | SEQ ID NO: 2357 | SEQ ID NO: 4723 | SEQ ID NO: 7089 |
| rs2075800 | SEQ ID NO: 2358 | SEQ ID NO: 4724 | SEQ ID NO: 7090 |
| rs707926 | SEQ ID NO: 2359 | SEQ ID NO: 4725 | SEQ ID NO: 7091 |
| rs387608 | SEQ ID NO: 2360 | SEQ ID NO: 4726 | SEQ ID NO: 7092 |
| rs2071888 | SEQ ID NO: 2361 | SEQ ID NO: 4727 | SEQ ID NO: 7093 |
| rs2073524 | SEQ ID NO: 2362 | SEQ ID NO: 4728 | SEQ ID NO: 7094 |
| rs805306 | SEQ ID NO: 2363 | SEQ ID NO: 4729 | SEQ ID NO: 7095 |
| rs486416 | SEQ ID NO: 2364 | SEQ ID NO: 4730 | SEQ ID NO: 7096 |
| rs652888 | SEQ ID NO: 2365 | SEQ ID NO: 4731 | SEQ ID NO: 7097 |
| rs2075798 | SEQ ID NO: 2366 | SEQ ID NO: 4732 | SEQ ID NO: 7098 |

TABLE 3

| SNP Name | Allele sequence of first ASO | | Allele sequence of second ASO | | Locus sequence of LSO | |
|---|---|---|---|---|---|---|
| rs2395256 | TTTCAGCAAACACCTTTTTCCT | SEQ ID NO: 1 | TTTTCAGCAAACACCTTTTTCCCC | SEQ ID NO: 2367 | GACTGCATTCACAAACCTTACTA | SEQ ID NO: 4733 |
| rs1894411 | TGCAAAATTGCCTTCATAGAGGCTA | SEQ ID NO: 2 | TGCAAAATTGCCTTCATAGAGGCTG | SEQ ID NO: 2368 | ATTAATTTATAGTTCCAGCAGCA | SEQ ID NO: 4734 |
| rs2621414 | GAGGCCAGAGCAAATTTCCCCA | SEQ ID NO: 3 | GAGGCCAGAGCAAATTTCCCCG | SEQ ID NO: 2369 | ACTCCATTTCAAATACTTCAAATT | SEQ ID NO: 4735 |
| rs1264298 | CTTGGATTTCCCACAATATGGCTA | SEQ ID NO: 4 | CTTGGATTTCCCACAATATGGCTG | SEQ ID NO: 2370 | CCCTCCTCTCTTCCTATAGAATC | SEQ ID NO: 4736 |
| rs1264307 | TGGGATAACAGCTGAACTGGGATA | SEQ ID NO: 5 | TGGGATAACAGCTGAACTGGGATG | SEQ ID NO: 2371 | GTGGAGTTGATGACAGGAGTTAT | SEQ ID NO: 4737 |
| rs241454 | AGCAGACACTGGTAGGAACAAGGAA | SEQ ID NO: 6 | AGCAGACACTGGTAGGAACAAGGAG | SEQ ID NO: 2372 | GATATAGGAAGGCAATCTCATG | SEQ ID NO: 4738 |
| rs3134996 | TCTGGGGGCAGCATTTCTAGTTT | SEQ ID NO: 7 | TCTGGGGGCAGCATTTCTAGTTA | SEQ ID NO: 2373 | ATTACCATTGGCATTAAGGAAG | SEQ ID NO: 4739 |
| rs2294882 | TATGCCAGGCACGATGCTGAATA | SEQ ID NO: 8 | TATGCCAGGCACGATGCTGAATG | SEQ ID NO: 2374 | GTTATGGGAATTATTTCATTTTTT | SEQ ID NO: 4740 |
| rs2001100 | GGGAAGGACTGAGAGTCCCATTA | SEQ ID NO: 9 | GGGAAGGACTGAGAGTCCCATTT | SEQ ID NO: 2375 | GAAACTTCCCAAATCTTAACTG | SEQ ID NO: 4741 |
| rs2273017 | AATCTTGGCATCCCTCCCTCCA | SEQ ID NO: 10 | AATCTTGGCATCCCTCCCTGCG | SEQ ID NO: 2376 | GGTTTTCACAGGAAATCTTCAT | SEQ ID NO: 4742 |
| rs3817969 | TGAGGTTCACTGAGATTTGTTGGCA | SEQ ID NO: 11 | TGAGGTTCACTGAGATTTGTTGGCG | SEQ ID NO: 2377 | GAACAATTTCCTATGATCTTTAAAA | SEQ ID NO: 4743 |
| rs761189 | AAGGAAAGAACAGAGCAGCAGAATCA | SEQ ID NO: 12 | AAGGAAAGAACAGAGCAGCAGAATCT | SEQ ID NO: 2378 | CAATGAAAGAAGACTTATAGGGAC | SEQ ID NO: 4744 |
| rs3129736 | CCCTGCGAGTTGAGGCATGA | SEQ ID NO: 13 | CCCTGCGAGTTGAGGCATGG | SEQ ID NO: 2379 | GAGCAGAAGAGGACAGTTTTTAC | SEQ ID NO: 4745 |
| rs3763350 | TTCCCCTTTATCCCCTGCTAATCA | SEQ ID NO: 14 | TTCCCCTTTATCCCCTGCTAATCG | SEQ ID NO: 2380 | CCTGTCTTTATTCTTTTATCTGAAG | SEQ ID NO: 4746 |
| rs3135374 | TGGATTTGGCAACATGGAGCTAAA | SEQ ID NO: 15 | TGGATTTGGCAACATGGAGCTAAC | SEQ ID NO: 2381 | CATGTCAGTGACAAGGAGTTC | SEQ ID NO: 4747 |
| rs2294880 | ACAGGAAGCAAAGGGAAGCTCCA | SEQ ID NO: 16 | ACAGGAAGCAAAGGGAAGCTCCG | SEQ ID NO: 2382 | CTTTCCGTGTTGGTTAATTGT | SEQ ID NO: 4748 |
| rs397081 | CAAAGTTGTCCTGGGTTGACTCAA | SEQ ID NO: 17 | CAAAGTTGTCCTGGGTTGACTCAG | SEQ ID NO: 2383 | AAGGTAGGATGAGCATTCTGAG | SEQ ID NO: 4749 |
| rs3021302 | GATAATCAGGAAGGGCAAGGTTATGT | SEQ ID NO: 18 | GATAATCAGGAAGGGCAAGGTTATGC | SEQ ID NO: 2384 | GCAATAAGATCTTAACCCACAGT | SEQ ID NO: 4750 |
| rs3129871 | TTCCAGTCCTTTCTGTGGAAAGAAA | SEQ ID NO: 19 | TTCCAGTCCTTTCTGTGGAAAGAAC | SEQ ID NO: 2385 | TAACATTCTCCTTTTCATAACACT | SEQ ID NO: 4751 |
| rs365053 | CCTCCCTGTTGGTTACTCTTCATCTCT | SEQ ID NO: 20 | CCTCCCTGTTGGTTTACTCTTCATCTCG | SEQ ID NO: 2386 | CAACCTCTTACCATTGTAGTGTC | SEQ ID NO: 4752 |
| rs1264308 | GGTGAGGGAATGCCAGTTTATGTTT | SEQ ID NO: 21 | GGTGAGGGAATGCCAGTTTATGTTC | SEQ ID NO: 2387 | TGTTTACCTGGCAGTCTACAG | SEQ ID NO: 4753 |
| rs3806156 | CAAGGACATTTTGTGCTGGATTAAT | SEQ ID NO: 22 | CAAGGACATTTTGTGCTGGATTAAG | SEQ ID NO: 2388 | ACACTGGTTTGTCTAGAGATTTTT | SEQ ID NO: 4754 |
| rs2253588 | GAACAAAGGGTTAAAGGGCGCG | SEQ ID NO: 23 | GAACAAAGGGTTAAAGGGCGCC | SEQ ID NO: 2389 | TGTAAGAGAGCTCTCCATTCC | SEQ ID NO: 4755 |
| rs2074512 | TCTCTGCATATCACCATCATTGTCCT | SEQ ID NO: 24 | TCTCTGCATATCACCATCATTGTCCC | SEQ ID NO: 2390 | GTCTTTGTCTCTAGTACTGAACCTG | SEQ ID NO: 4756 |
| rs2076536 | TGCCTGCTTCCTTTTCAAATTCAA | SEQ ID NO: 25 | TGCCTGCTTCCTTTTCAAATTCAG | SEQ ID NO: 2391 | ATTTTTTCTTTCACTCTTATTGATT | SEQ ID NO: 4757 |
| rs1988563 | GGGTGGTAACCCAGCCACTCTTT | SEQ ID NO: 26 | GGGTGGTAACCCAGCCACTCTTA | SEQ ID NO: 2392 | TTCCCTTACTCTTCCTTCCAT | SEQ ID NO: 4758 |
| rs2253705 | GGGGTGGGACACAGAGTGGAAA | SEQ ID NO: 27 | GGGGTGGGACACAGAGTGGAAG | SEQ ID NO: 2393 | GAGGGGACTTCCTTCTATTTTC | SEQ ID NO: 4759 |
| rs1894412 | ATAGCAGGCAGCCTGGGTTTTCA | SEQ ID NO: 28 | ATAGCAGGCAGCCTGGGTTTTCG | SEQ ID NO: 2394 | CCCAGTCTTCTTCTGTTTTCA | SEQ ID NO: 4760 |
| rs2071554 | CCTTGAGTCATGGAGGAATCCAGTT | SEQ ID NO: 29 | CCTTGAGTCATGGAGGAATCGAGTC | SEQ ID NO: 2395 | GGTCAGATTCACTAGCAGAGC | SEQ ID NO: 4761 |
| rs2395264 | GGTTCATGCAAAAAAGCAACATTAGT | SEQ ID NO: 30 | GGTTCATGCAAAAAAGCAACATTAGC | SEQ ID NO: 2396 | GTAATATTTTGGGGGGACTACT | SEQ ID NO: 4762 |
| rs2071290 | GGGCAAGTAAGGAGACTGATACGAAGA | SEQ ID NO: 31 | GGGCAAGTAAGGAGACTGATACGAAGG | SEQ ID NO: 2397 | AGTAGGAAAGAAAAGGTACAGAGG | SEQ ID NO: 4763 |
| rs885912 | CCGAGGCTCTGGCACTCACTT | SEQ ID NO: 32 | CCGAGGGTCTGGCACTCACTG | SEQ ID NO: 2398 | CCTCCTCTGTAAATAAGGGGT | SEQ ID NO: 4764 |
| rs2073717 | CCTTTGGTGAAAATAAACACACATGGG | SEQ ID NO: 33 | CCTTTGGTGAAAATAAACACACATGGC | SEQ ID NO: 2399 | TAGAAAGATGGAGAATTTGGGTA | SEQ ID NO: 4765 |
| rs3873444 | CAAGGAAAGGAGTCAGGGGCTAAT | SEQ ID NO: 34 | CAAGGAAAGGAGTCAGGGGCTAAC | SEQ ID NO: 2400 | AAGGAATATTGTCCACCCCTA | SEQ ID NO: 4766 |
| rs261949 | GTGTGAAGGAACCTGGCCCAT | SEQ ID NO: 35 | GTGTGAAGGAACCTGGCCCAC | SEQ ID NO: 2401 | GAAGTTGCCCTGTAAATTCAG | SEQ ID NO: 4767 |
| rs2187818 | TGATCTTATTCCAGACCAGCGATACTA | SEQ ID NO: 36 | TGATCTTATTCCAGACCAGCGATACTC | SEQ ID NO: 2402 | GTAAGATACTCTTTCACAATGCC | SEQ ID NO: 4768 |
| rs2071550 | CAACCCCAATCCCACAGGTTTAAT | SEQ ID NO: 37 | CAACCCCAATCCCACAGGTTTAAG | SEQ ID NO: 2403 | CTGAAGGAGGAGAGAGGAAAG | SEQ ID NO: 4769 |
| rs1573649 | TCTGCAGAGCCATCTTCCAAGACA | SEQ ID NO: 38 | TCTGCAGAGCCATCTTCCAAGACG | SEQ ID NO: 2404 | AAGTGAGACCAAGGAAAAAGC | SEQ ID NO: 4770 |
| rs2395110 | TCTTGATCTCTGCCATCTGGAGGA | SEQ ID NO: 39 | TCTTTGATCTCTGCCATCTGGAGGG | SEQ ID NO: 2405 | GGAAATTCTTGGGTTCAAGTC | SEQ ID NO: 4771 |
| rs2073046 | AACAACTTTGGCATCTCTCCAGAT | SEQ ID NO: 40 | AACAACTTTGGCATCTCTCCAGAC | SEQ ID NO: 2406 | TTTCCATGGAAAGTCTTCTTC | SEQ ID NO: 4772 |
| rs2073047 | TTGTGCATAAGCTACAAGAGCTGAT | SEQ ID NO: 41 | TTGTGCATAAGCTACAAGAGCTGAC | SEQ ID NO: 2407 | ATGATAAGTGACTGTGTCAATCAC | SEQ ID NO: 4773 |
| rs743862 | TGGTTGAAAGATGTTTTGAGAGCTCTT | SEQ ID NO: 42 | TGGTTGAAAGATGTTTTGAGAGCTCTC | SEQ ID NO: 2408 | GATCAACTCACCAGATAGATGAT | SEQ ID NO: 4774 |
| rs2074488 | CAGACAGTGAGCTCGCCCTGT | SEQ ID NO: 43 | CAGAGAGTGAGCTCGCCCTGG | SEQ ID NO: 2409 | AATGGACGTGTAGAGTCAGGA | SEQ ID NO: 4775 |
| rs2072895 | AGAGTCCCAGGCGCCTTTACCG | SEQ ID NO: 44 | AGAGTCCCAGGCGCCTTTACCC | SEQ ID NO: 2410 | GGTTCATTTTCAGTTTAGGCC | SEQ ID NO: 4776 |
| rs2076537 | TTGCAGAAAGGTTGTGGAAAAGAA | SEQ ID NO: 45 | TTGCAGAAAGGTTGTGGAAAAGAG | SEQ ID NO: 2411 | ATAGAAATGAGTCACAACTTATTTCC | SEQ ID NO: 4777 |
| rs2072110 | CGTCCAGTGTCACAGGGGCT | SEQ ID NO: 46 | CGTCCAGTGTCACAGGGGCC | SEQ ID NO: 2412 | TGTGAAGATGAGGAGAAAGAG | SEQ ID NO: 4778 |
| rs13501 | TCAAAAGAGTTGCCTATACTCCCTGAT | SEQ ID NO: 47 | TCAAAAGAGTTGCCTATACTCCCTGAC | SEQ ID NO: 2413 | CCACTTATCTTCCTACTATCCTCT | SEQ ID NO: 4779 |
| rs2072896 | CACTCCCATTGGGCGTCGG | SEQ ID NO: 48 | CACTCCCATTGGGCGTCGC | SEQ ID NO: 2414 | TTTCTAGAGAAGCCAATCAGTG | SEQ ID NO: 4780 |
| rs2581 | GCTTCCCTTCCCATTCAACACAA | SEQ ID NO: 49 | GCTTCCCTTCCCATTCAACACAC | SEQ ID NO: 2415 | CACACATTCTTGCTCTACCC | SEQ ID NO: 4781 |
| rs2023472 | AGGCTCCCTGTGATGGAGTCA | SEQ ID NO: 50 | AGGCTCCCTGTGATGGAGTCG | SEQ ID NO: 2416 | GTCTTGATTTTGCTTCACTGAG | SEQ ID NO: 4782 |
| rs2006165 | TCATTGAGCTCATACCACATGCCA | SEQ ID NO: 51 | TCATTGAGCTCATACCACATGCCG | SEQ ID NO: 2417 | TCCACGAGTCAGGTACTAGG | SEQ ID NO: 4783 |
| rs2395533 | CCAGCATCTCATACACCACCATGT | SEQ ID NO: 52 | CCAGCATCTCATACACCACCATGC | SEQ ID NO: 2418 | CCTACTGCAAGTTCCATTG | SEQ ID NO: 4784 |
| rs2269703 | GGAGCCGAAAAGGGCGGTA | SEQ ID NO: 53 | GGAGCCGAAAAGGGCGGTG | SEQ ID NO: 2419 | CCGGAGTATTCTAAGTAAAGTGTT | SEQ ID NO: 4785 |
| rs259919 | TGCACACCACCTAGATTCTGTCAAAA | SEQ ID NO: 54 | TGCACACCACCTAGATTCTGTCAAAG | SEQ ID NO: 2420 | GAAGTCAGAAATTTATAATGTGATCA | SEQ ID NO: 4786 |
| rs1265760 | GGCCTCTACTCACTCTCCCCCAA | SEQ ID NO: 55 | GGCCTCTACTCACTCTCCCCCAG | SEQ ID NO: 2421 | ACCACACAATTTATTGTAATAAAAT | SEQ ID NO: 4787 |
| rs241428 | GGATTTTCCAACTCGAAGGATGAA | SEQ ID NO: 56 | GGATTTTCCAACTCGAAGGATGAC | SEQ ID NO: 2422 | GATGGGAATCACATGACTCTG | SEQ ID NO: 4788 |
| rs3763316 | GCACCTTGCCACCTGAAACTTACT | SEQ ID NO: 57 | GCACCTTGCCACCTGAAACTTACC | SEQ ID NO: 2423 | GTCACCTCCCCTTAGACTGAA | SEQ ID NO: 4789 |
| rs8084 | CCACTGGTGGGGTGAACTTGTCT | SEQ ID NO: 58 | CCACTGGTGGGGTGAACTTGTCG | SEQ ID NO: 2424 | TGAAACAGATGAGGACGTTG | SEQ ID NO: 4790 |
| rs1042337 | ATGGGCTCAGGAGCCCCAA | SEQ ID NO: 59 | ATGGGCTCAGGAGCCCCAG | SEQ ID NO: 2425 | GTGCTCTACCACACAGGTGTA | SEQ ID NO: 4791 |
| rs2269713 | GCCAGAAATACACCACCTACAGTACAAAT | SEQ ID NO: 60 | GCCAGAAATACACCACCTACAGTACAAAC | SEQ ID NO: 2426 | ATAATCTAAAAACAAGAGGGTGG | SEQ ID NO: 4792 |
| rs2239524 | GGGAGGGTGGGAGGAAGATCTTA | SEQ ID NO: 61 | GGGAGGGTGGGAGGAAGATCTTC | SEQ ID NO: 2427 | AGTGCCCACTATGTGTTAGGA | SEQ ID NO: 4793 |
| rs3115569 | AGGAATAAAAAGTAGGAAGCCAAAAT | SEQ ID NO: 62 | AGCAATAAAAAGTAGGAAGCCAAAAC | SEQ ID NO: 2428 | GAAGCACAGTACTATTTACAACCA | SEQ ID NO: 4794 |
| rs241448 | CTGCAGAAGCTTGCCCAGCTCT | SEQ ID NO: 63 | CTGCAGAAGCTTGCCCAGCTCC | SEQ ID NO: 2429 | GGAGGGACAGGACCTCTATT | SEQ ID NO: 4795 |
| rs2071475 | TTTCACGGTGTCTCATTTTTCCTCT | SEQ ID NO: 64 | TTTCACGGTGTCTCATTTTTCCTCC | SEQ ID NO: 2430 | TTCCTAGTGCAACCAGAGGT | SEQ ID NO: 4796 |
| rs365066 | GTGACAGTTTGGCCGTTGCGT | SEQ ID NO: 65 | GTGACAGTTTGGCCGTTGCGC | SEQ ID NO: 2431 | GCCAGGTGATATTGATCACAG | SEQ ID NO: 4797 |
| rs2022059 | AGTTGAGCAATCCGGGGGGG | SEQ ID NO: 66 | AGTTGAGCAATCCGGGGGGC | SEQ ID NO: 2432 | CCCACATACCAGTTTTCTCCT | SEQ ID NO: 4798 |
| rs2071474 | GGATTTAGACTTTCTGAGGTACTCCCAT | SEQ ID NO: 67 | GGATTTAGACTTTCTGAGGTACTCCCAC | SEQ ID NO: 2433 | ATTACTGCTTTCTCTTTGAGGG | SEQ ID NO: 4799 |
| rs3129745 | TTTGACTTGTTTTGGTTTGGTTTCCA | SEQ ID NO: 68 | TTTGACTTGTTTTGGTTTGGTTTCCG | SEQ ID NO: 2434 | TTGATACTGAGGATGAAGGGA | SEQ ID NO: 4800 |
| rs2213572 | CTCTGCGTGGTAGGTAAATTTTATGTT | SEQ ID NO: 69 | CTCTGCGTGGTAGGTAAATTTTATGTC | SEQ ID NO: 2435 | TTTCACATTCACAGGTATTTTTAT | SEQ ID NO: 4801 |
| rs2286656 | TCAGTTGCAAAGTCATACCCGGT | SEQ ID NO: 70 | TCAGTTGCAAAGTCATACCCGGC | SEQ ID NO: 2436 | CTGCGGATCCAGAAGTACAG | SEQ ID NO: 4802 |
| rs2070121 | GAGTGGCATTTGCAGCCTTCCTAT | SEQ ID NO: 71 | GAGTGGCATTGCAGCCTTCCTAC | SEQ ID NO: 2437 | TGGGCTAATCTTCCTTCTG | SEQ ID NO: 4803 |
| rs3117098 | TTTCACTTACTTTTGAGTCTTCCTTCCT | SEQ ID NO: 72 | TTTCACTTACTTTTGAGTCTTCCTTCCC | SEQ ID NO: 2438 | GTGAATGTAGCTCTTTGCTTGT | SEQ ID NO: 4804 |
| rs926594 | AGTGTCTCAATTCCTTTGCCCAA | SEQ ID NO: 73 | AGTGTCTCAATTCCTTTGCCCAC | SEQ ID NO: 2439 | ACTCATACCTATAAATGTCTTAAGTCA | SEQ ID NO: 4805 |
| rs2621393 | AGAGATTTCCCAGAACTCCGATAAC | SEQ ID NO: 74 | AGAGATTTCCCAGAACTCCGATAAG | SEQ ID NO: 2440 | TTTATTTTAACCTTTTTACCTCTGA | SEQ ID NO: 4806 |
| rs7192 | GCTGCATTGCTTTTGCGCAA | SEQ ID NO: 75 | GCTGCATTGCTTTTGCGCAC | SEQ ID NO: 2441 | CCCTTGATGATGAAGATGGT | SEQ ID NO: 4807 |
| rs14004 | CCATTTTCTTCTGGGCGCTCTT | SEQ ID NO: 76 | CCATTTTCTTCTTGGGCGCTCTG | SEQ ID NO: 2442 | TGGGAGTCAGTAGAGCTCG | SEQ ID NO: 4808 |
| rs3134943 | TTGACAGATTTGCCGGCTTCCT | SEQ ID NO: 77 | TTGACAGATTTGCCGGCTTCCC | SEQ ID NO: 2443 | TCTGACTTTTTCTGCCTCC | SEQ ID NO: 4809 |
| rs2213585 | TCTAACCCCATCTGCTCCCCTCT | SEQ ID NO: 78 | TCTAACCCCATCTGCTCCGCTCC | SEQ ID NO: 2444 | TCCCATGCATTCTGAGATC | SEQ ID NO: 4810 |
| rs2072894 | CTGCCCCTACCCAGCAGTGAT | SEQ ID NO: 79 | CTGCCCCTACCCAGCAGTGAC | SEQ ID NO: 2445 | TCTCCATTCCAGTCCAAGT | SEQ ID NO: 4811 |
| rs2286655 | GCCAGAGAAAAGGGGTGGGAT | SEQ ID NO: 80 | GCCAGAGAAAAGGGGTGGGAC | SEQ ID NO: 2446 | TCATGGTCCCTGAGAGTGAC | SEQ ID NO: 4812 |
| rs886420 | AGTGGGGTCTTGGGGCAGTAGT | SEQ ID NO: 81 | AGTGGGGTCTTGGGGCAGTAGC | SEQ ID NO: 2447 | AGAACTGAATACTTGGGTCTCTC | SEQ ID NO: 4813 |
| rs2074504 | GAGGTCCATTGACGCCTGGA | SEQ ID NO: 82 | GAGGTCCATTGACGCCTGGG | SEQ ID NO: 2448 | GGTAGAAGGCACAGAGGTC | SEQ ID NO: 4814 |
| rs2301270 | ATGCTCCTGCCCCTGCCA | SEQ ID NO: 83 | ATGCTCCTGCCCCTGCCC | SEQ ID NO: 2449 | GTGAGACACCTCACTCCCTCT | SEQ ID NO: 4815 |
| rs3763310 | GACACAGAGATGCCGTGGAAAA | SEQ ID NO: 84 | GACACAGAGATGCCGTGGAAAG | SEQ ID NO: 2450 | AAAGATAGTCGGGAAAATATCTG | SEQ ID NO: 4816 |
| rs241446 | CTCCAGCTGTGGCAGTACAGCT | SEQ ID NO: 85 | CTCCAGCTGTGGCAGTACAGCC | SEQ ID NO: 2451 | CTTTATCTACTGCCCTTTCCTAC | SEQ ID NO: 4817 |
| rs3129876 | TGAAGTAAGTCAAATTTGGTTTAGAGGA | SEQ ID NO: 86 | TGAAGTAAGTCAAATTTGGTTTAGAGGG | SEQ ID NO: 2452 | CAAAACTGAGTTGTGTATTGATG | SEQ ID NO: 4818 |
| rs1043483 | GGGTCCTGGCTGTGAGGCTA | SEQ ID NO: 87 | GGGTCCTGGCTGTGAGGCTG | SEQ ID NO: 2453 | CAAGCTGGTTCTGCAACTT | SEQ ID NO: 4819 |
| rs2286405 | CGCTCGATGCAGTCACAATGT | SEQ ID NO: 88 | CGCTCGATGCAGTCACAATGC | SEQ ID NO: 24S4 | ACTGCCTGTTTTTAGGAAGTATC | SEQ ID NO: 4820 |
| rs2284175 | AACCGTTTACTTAGAAGACCCAGATAAA | SEQ ID NO: 89 | AACCGTTTACTTAGAAGACCGAGATAAG | SEQ ID NO: 2455 | ACAGTTTTTAAGACACCAACTATTT | SEQ ID NO: 4821 |
| rs1362122 | TTAGAGCAATTTCCAACCCTTGCT | SEQ ID NO: 90 | TTAGAGCAATTTCCAACCCTTGCC | SEQ ID NO: 2456 | AGGCTGCTACACTTTTACCTATG | SEQ ID NO: 4822 |
| rs1265155 | AGCTGCTCCGGTTCCCCAA | SEQ ID NO: 91 | AGCTGCTCCGGTTCCCCAG | SEQ ID NO: 2457 | AACTCACAGACAACCGTGGT | SEQ ID NO: 4823 |
| rs2267641 | CCCAGCAGCCGAATGATGTTT | SEQ ID NO: 92 | CCCAGCAGCCGAATGATGTTG | SEQ ID NO: 2458 | ACATGATCTTCACCTCTTTCAG | SEQ ID NO: 4824 |
| rs2647089 | AGTTGGAATAAGCCCACTATAGTCTGTA | SEQ ID NO: 93 | AGTTGGAATAAGCCCACTATAGTCTGTG | SEQ ID NO: 2459 | CTTACACTAATTATAATGGGAAACA | SEQ ID NO: 4825 |
| rs2240619 | GAGGGCGATATTCTGGTGGTGT | SEQ ID NO: 94 | GAGGGCGATATTCTGGTGGTGG | SEQ ID NO: 2460 | TCCATTTTCTTCCCCTCCT | SEQ ID NO: 4826 |
| rs1061808 | AGGTGCTGAGGAAAGCTCCCAT | SEQ ID NO: 95 | AGGTGCTGAGGAAAGCTCCCAG | SEQ ID NO: 2461 | AGTGCTTCAGGTATCAGCTTC | SEQ ID NO: 4827 |
| rs2249099 | GGTCAGGTGCTGAGGCGCA | SEQ ID NO: 96 | GGTCAGGTGCTGAGGCGCC | SEQ ID NO: 2462 | AGGAGAGGACATGGCTCACT | SEQ ID NO: 4828 |
| rs1264309 | TGGAGTCTTGCCTTTGCTCTCCTT | SEQ ID NO: 97 | TGGAGTCTTGCCTTTGCTCTCCTC | SEQ ID NO: 2463 | GTCTGACTACAAATCCCGAAAG | SEQ ID NO: 4829 |
| rs3763305 | CAGAGGGAGGGGAGCTACCAT | SEQ ID NO: 98 | CAGAGGGAGGGGAGCTACCAC | SEQ ID NO: 2464 | AAAAACTGAGGCACGATTTTAC | SEQ ID NO: 4830 |
| rs2073724 | ACCGGTGGCGGGTTCTCAA | SEQ ID NO: 99 | ACCGGTGGCGGGTTCTCAG | SEQ ID NO: 2465 | AGGCTCACTCTCATCATCCA | SEQ ID NO: 4831 |
| rs2582 | GGCACTGAGCACGCAGGGT | SEQ ID NO: 100 | GGCACTGAGCACGCAGGGG | SEQ ID NO: 2466 | TGTCACAAACCCATGAGGAT | SEQ ID NO: 4832 |
| rs2074484 | GAACAAAGTGGGCGTCCGCT | SEQ ID NO: 101 | GAACAAAGTGGGCGTCCGCG | SEQ ID NO: 2467 | GGATTACTAGGGCGGAGAG | SEQ ID NO: 4833 |
| rs3134945 | TCGCTTATATACTGGAGAGGCTAGGAGA | SEQ ID NO: 102 | TCGCTTATATACTGGAGAGGGTAGGAGC | SEQ ID NO: 2468 | CATACAGTCATACAGATAATCGGA | SEQ ID NO: 4834 |
| rs2269418 | CAACACCTCTGCAATCAAGAACTGAT | SEQ ID NO: 103 | CAACACCTCTGCAATCAAGAACTGAG | SEQ ID NO: 2469 | TGTCTGTGTGGTTTTGATT | SEQ ID NO: 4835 |
| rs3749968 | TGGATTTCATCTCTCAATAATTCTGCTTAT | SEQ ID NO: 104 | TGGATTTCATCTCTCAATAATTCTGCTTAC | SEQ ID NO: 2470 | TGTTATTTCATAATATTGTTCAGTTT | SEQ ID NO: 4836 |
| rs2856705 | GACATCTTTCTACTCAGTTACCATTAGGAA | SEQ ID NO: 105 | GACATCTTTCTACTCAGTTACCATTAGGAG | SEQ ID NO: 2471 | GAGAAAAATATACTAAGAAAGGCAA | SEQ ID NO: 4837 |
| rs2071283 | CAGAGGCACTGGAAGGAGCCT | SEQ ID NO: 106 | CAGAGGCACTGGAAGGAGCCC | SEQ ID NO: 2472 | GGGTGTTATGGCAGGAGGT | SEQ ID NO: 4838 |
| rs2239518 | ATGTGGAGGTGGAATGCCCACA | SEQ ID NO: 107 | ATGTGGAGGTGGAATGCCCACG | SEQ ID NO: 2473 | GAGGCCAAGGGTCACTTC | SEQ ID NO: 4839 |
| rs2240804 | GGCCAGATCCCTTCCCCACA | SEQ ID NO: 108 | GGCCAGATCCCTTCCCCACG | SEQ ID NO: 2474 | TGATCTTGGAGTAGGCGC | SEQ ID NO: 4840 |
| rs2076525 | GCACCTCGTATTTTTATCAAGAGACATTA | SEQ ID NO: 109 | GCACCTCGTATTTTTATCAAGAGACATTG | SEQ ID NO: 2475 | TAGCAATATGTATTGAAAGCCAT | SEQ ID NO: 4841 |
| rs3806157 | TGCAAATTGAAGAGATGAAACAGTTAT | SEQ ID NO: 110 | TGCAAATTGAAGAGATGTTTAACAGTTAG | SEQ ID NO: 2476 | TATGTTATTATGTTTGATCATTTTATG | SEQ ID NO: 4842 |
| rs2187823 | CAGGATCCAAACAGTGATCAGTTCCT | SEQ ID NO: 111 | CAGGATCCAAACAGTGATCAGTTCCG | SEQ ID NO: 2477 | GATCCCTGGTTTATTCATGTAA | SEQ ID NO: 4843 |
| rs3129858 | TGCAGCATGGACCACTGCTCA | SEQ ID NO: 112 | TGCAGCATGGACCACTGCTCG | SEQ ID NO: 2478 | GTACAGATGCTCTCCAACTTAAC | SEQ ID NO: 4844 |
| rs3135396 | TCCTGTGTTAATAGAGGGCTCCACA | SEQ ID NO: 113 | TCCTGTGTTAATAGAGGGCTCCACG | SEQ ID NO: 2479 | TGACTGGACTCATCTTTAACCT | SEQ ID NO: 4845 |
| rs1736917 | GTCGCTGCTGTGCTGTGGAGA | SEQ ID NO: 114 | GTCGCTGCTGTGCTGTGGAGG | SEQ ID NO: 2480 | AGAAGAGCTCAGGTAAGGAAG | SEQ ID NO: 4846 |
| rs3213485 | GGAGATTCGTAGAAACTGGCAAACTTT | SEQ ID NO: 115 | GGAGATTCGTAGAAACTGGCAAACTTC | SEQ ID NO: 2481 | CCCCTAAGTCTTAACCCTCATA | SEQ ID NO: 4847 |
| rs184003 | CCCTCTGCCCTCCCTGTTGA | SEQ ID NO: 116 | CCCTCTGOCCTCCCTGTTGC | SEQ ID NO: 2482 | AGTTATGGTTCACCCTACCTC | SEQ ID NO: 4848 |
| rs2647086 | CAACCTTTCTGGCCAGAGGAACC | SEQ ID NO: 117 | CAACCTTTCTGGCCAGAGGAACG | SEQ ID NO: 2483 | GTAAGAGGGACCTATGGGAC | SEQ ID NO: 4849 |
| rs2235383 | TCTGTCCTTCATTTTCACAGGAGCT | SEQ ID NO: 118 | TCTGTCCTTCATTTTCACAGGAGCC | SEQ ID NO: 2484 | TACAAATGAGTAGGTGCTGGC | SEQ ID NO: 4850 |
| rs3817970 | CATTCTATTAACAGCCAAACAGGAAGAT | SEQ ID NO: 119 | CATTCTATTAACAGCCAAACAGGAAGAC | SEQ ID NO: 2485 | AGTGTTTCACGGACATAAGAAAT | SEQ ID NO: 4851 |
| rs2213574 | TCTCAGAGGAAATGGGTCCACAGA | SEQ ID NO: 120 | TCTCAGAGGAAATGGGTCCACAGG | SEQ ID NO: 2486 | ACCTGAGGGCTCTAAACAGCTA | SEQ ID NO: 4852 |
| rs2858857 | GCCTCTTCCCTTTGCAGGGT | SEQ ID NO: 121 | GCCTCTTCCCTTTGCAGGGC | SEQ ID NO: 2487 | AAAAGCCAGGAGCTACACAC | SEQ ID NO: 4853 |
| rs2227139 | GATGAGAGGAAGGAATATTTGAAACACA | SEQ ID NO: 122 | GATGAGAGGAAGGAATATTTGAAACACG | SEQ ID NO: 2488 | TGAACTGTTGCTCATTTGTCTAT | SEQ ID NO: 4854 |
| rs709052 | CTGGTCTGGTCTCCACAAGCTCA | SEQ ID NO: 123 | CTGGTCTGGTCTCCACAAGCTCG | SEQ ID NO: 2489 | TGTCCTGAGTTTGGTCCTC | SEQ ID NO: 4855 |
| rs2239519 | GTGTTCCATCACCTGGGGAACT | SEQ ID NO: 124 | GTGTTCCATCACCTGGGGAACC | SEQ ID NO: 2490 | TCTCCTTCTCAGAAATGGG | SEQ ID NO: 4856 |
| rs3844313 | GCTCCTCTTGGCAGTGACTAGGGA | SEQ ID NO: 125 | GCTCCTCTTGGCAGTGACTAGGGG | SEQ ID NO: 2491 | AGTACTAATGGTTATAAAGCAATTAGA | SEQ ID NO: 4857 |
| rs2076534 | GATAGCAGGAGACCTCGTCAGAGAT | SEQ ID NO: 126 | GATAGCAGGAGACCTCGTCAGAGAG | SEQ ID NO: 2492 | AGCAAATAAATCACAAAGGA | SEQ ID NO: 4858 |
| rs2074490 | CACTCCCATTGGGTGTCGGA | SEQ ID NO: 127 | CACTCCCATTGGGTGTCGGG | SEQ ID NO: 2493 | TCTAGAGAAGCCAATCAGCGT | SEQ ID NO: 4859 |
| rs1049033 | GACTACAGCTGCAAGGACAACCAGA | SEQ ID NO: 128 | GACTACAGCTGCAAGGACAACCAGG | SEQ ID NO: 2494 | CAGCAACGATACCCATGAT | SEQ ID NO: 4860 |
| rs206019 | GCTCTCCAGTCAGTGCCGGT | SEQ ID NO: 129 | GCTCTCCAGTCAGTGCCGGC | SEQ ID NO: 2495 | TTGGTTACCTTGGCTCAGT | SEQ ID NO: 4861 |
| rs1265157 | AGATGGGAGGGCATATGAGAGCC | SEQ ID NO: 130 | AGATGGGAGGGCATATGAGAGCG | SEQ ID NO: 2496 | TTGGAAGGAAGTTTAGAGTTCAT | SEQ ID NO: 4862 |
| rs1044043 | TGTTTCACAATTGCCATGTGGATTA | SEQ ID NO: 131 | TGTTTCACAATTGCCATGTGGATTC | SEQ ID NO: 2497 | AGGCATAAAGGACTTGAAACTC | SEQ ID NO: 4863 |
| rs1615839 | CCACTCCGCCCACCTTCCC | SEQ ID NO: 132 | CCACTCCGCCCACCTTCCG | SEQ ID NO: 2498 | GCAGGTAGGTTTTGAGCAG | SEQ ID NO: 4864 |
| rs2071555 | CTTGTCATGCCTCACAGCAGTGA | SEQ ID NO: 133 | CTTGTCATGCCTCACAGCAGTGC | SEQ ID NO: 2499 | AATGGAGACTGGACATACCAG | SEQ ID NO: 4865 |
| rs241441 | CCCGCAGCCAGCTGGCTT | SEQ ID NO: 134 | CCCGCAGCCAGCTGGCTC | SEQ ID NO: 2500 | CCTTCTCCCCTACATCTGAG | SEQ ID NO: 4866 |
| rs1624278 | GGTGGGGCCAGGTTCTCAT | SEQ ID NO: 135 | GGTGGGGCCAGGTTCTCAC | SEQ ID NO: 2501 | CCCTCCAGTGGATGATTG | SEQ ID NO: 4867 |
| rs2395195 | GTTTGGAGGAATTTTTGGTTGTCAT | SEQ ID NO: 136 | GTTTGGAGGAATTTTTGGTTGTCAC | SEQ ID NO: 2502 | ACAGGGATTTCTTCTGATATTTAA | SEQ ID NO: 4868 |
| rs915687 | CCTTTCTGGCCTCTCACAGGACA | SEQ ID NO: 137 | CCTTTCTGGCCTCTCACAGGACG | SEQ ID NO: 2503 | TTTCTTCCCACAGATTGAAAA | SEQ ID NO: 4869 |
| rs1964995 | CCCTTTAACTGCAGCTCACATTATTTTT | SEQ ID NO: 138 | CCCTTTAACTGCAGCTCACATTATTTTC | SEQ ID NO: 2504 | TTATTCCTTGATTCTAAAGCAATT | SEQ ID NO: 4870 |
| rs3117226 | GCTTGCGGTGATAACCATTCAACA | SEQ ID NO: 139 | GCTTGCGGTGATAACCATTCAACG | SEQ ID NO: 2505 | ACATTCTATTGGTTTGTGTGTTT | SEQ ID NO: 4871 |
| rs2858333 | TTGAGGCAAAGTGGGAAGAGAAAA | SEQ ID NO: 140 | TTGAGGCAAAGTGGGAAGAGAAAG | SEQ ID NO: 2506 | TGAGCAAAAATATAAAGGGAAAT | SEQ ID NO: 4872 |
| rs3025644 | CAGACCTGCCTTTGGGTAGGGT | SEQ ID NO: 141 | CAGACCTGCCAAGGGTAGGGA | SEQ ID NO: 2507 | CAGATCACTTTCTAACTCTGGAA | SEQ ID NO: 4873 |
| rs3129939 | CTCCCATCAGCCCTGTTTTACCTA | SEQ ID NO: 142 | CTCCCATCAGCCCTGTTTTACCTG | SEQ ID NO: 2508 | CTTTTCACACTACTTATGCTTTGA | SEQ ID NO: 4874 |
| rs2534655 | AGGGGTAGGAAGGTTGTAGCCCT | SEQ ID NO: 143 | AGGGGTAGGAAGGTTGTAGCCCC | SEQ ID NO: 2509 | GAGAAGTTTTATAGTGGGGTCAGT | SEQ ID NO: 4875 |
| rs2144425 | CAAATTCTTGGGCATGAAGTCGT | SEQ ID NO: 144 | CAAATTCTTGGGCATGAAGTCGC | SEQ ID NO: 2510 | CATAACGACCTTTTACAATGTTT | SEQ ID NO: 4876 |
| rs2523969 | GATATGCTGGGCTCTGGCTTGT | SEQ ID NO: 145 | GATATGCTGGGCTCTGGCTTGA | SEQ ID NO: 2511 | TCTCCAGAATATCTCTCTGACCT | SEQ ID NO: 4877 |
| rs2746149 | TGGACTCCCGTGACTGAGACCA | SEQ ID NO: 146 | TGGACTCCCGTGACTGAGACCG | SEQ ID NO: 2512 | AAACCGAGTAGCTTAAACTACAGA | SEQ ID NO: 4878 |
| rs1265048 | CTACTGGTGGTCAGATGTGCCAT | SEQ ID NO: 147 | CTACTGGTGGTCAGATGTGCCAC | SEQ ID NO: 2513 | GAGTATAAGAAACCTTGGGAGAA | SEQ ID NO: 4879 |
| rs2535276 | CTACTGGTGGTCAGATGTGCCAT | SEQ ID NO: 148 | CTACTGGTGGTCAGATGTGCCAC | SEQ ID NO: 2514 | GAGTATAAGAAACCTTGGGAGAA | SEQ ID NO: 4880 |
| rs3129267 | TAAGCTGGCAGTCCTAACCCCAG | SEQ ID NO: 149 | TAAGCTGGCAGTCCTAACCCCAC | SEQ ID NO: 2515 | GTGTTTATTCTACTCCAGGTGTC | SEQ ID NO: 4881 |
| rs3095314 | TGGCCACATGGAGCAAGTTTAAT | SEQ ID NO: 150 | TGGCCACATGGAGCAAGAAAAC | SEQ ID NO: 2516 | ACCAAAGATTTGGAGCTTTAAT | SEQ ID NO: 4882 |
| rs2844720 | CTCAAGCTGCAAACAGGATCCAT | SEQ ID NO: 151 | CTCAAGCTGCAAACAGGATCCAC | SEQ ID NO: 2517 | AAATAGCCAAAAGACAGTGTTC | SEQ ID NO: 4883 |
| rs2294476 | GCAGAGTTTGTGCAGACACACAGAT | SEQ ID NO: 152 | GCAGAGTTTGTGCAGACACACAGAC | SEQ ID NO: 2518 | CTCTGGGGAGTATATGTTTGAAT | SEQ ID NO: 4884 |
| rs2394517 | ATGAGGGTCAGCTCATTTGCCTA | SEQ ID NO: 153 | ATGAGGGTCAGCTCATTTGCCTG | SEQ ID NO: 2519 | GTATCAACACATGATAATCGCA | SEQ ID NO: 4885 |
| rs916571 | CAAAAGCACACAGAGACCTCCTGT | SEQ ID NO: 154 | CAAAAGCACACAGAGACCTCCTGC | SEQ ID NO: 2520 | TTGTGATTAGATTTTCATTGCAT | SEQ ID NO: 4886 |
| rs3129941 | TTTTGTTAACAAGATGGGCACGAT | SEQ ID NO: 155 | TTTTGTTAACAAGATGGGCACGAC | SEQ ID NO: 2521 | TAAGCAAAGTGGTGAGTAATAATG | SEQ ID NO: 4887 |
| rs3817659 | CTGCATCCAGTGGAACTAAAGCCA | SEQ ID NO: 156 | CTGCATCCAGTGGAACTAAAGCCG | SEQ ID NO: 2522 | TCCAGGATTATCTAAAACTGAG | SEQ ID NO: 4888 |
| rs2596449 | CGGGATGATAGCAGCTCTCCTACA | SEQ ID NO: 157 | CGGGATGATAGCAGCTCTCCTACG | SEQ ID NO: 2523 | AATAATGCTTATATGACAAGGGC | SEQ ID NO: 4889 |
| rs176250 | ACTGATGGGTGGGGAATGTTGAA | SEQ ID NO: 158 | ACTGATGGGTGGGGAATGTTGAG | SEQ ID NO: 2524 | ACAGTGGTAGGTATCTCAGGTG | SEQ ID NO: 4890 |
| rs6457116 | CAGGTGTCTGGAGTGCAGGTACAT | SEQ ID NO: 159 | CAGGTGTCTGGAGTGCAGGTACAC | SEQ ID NO: 2525 | TGGGACAGTTTCAACAAACTAT | SEQ ID NO: 4891 |
| rs2524070 | TGGAGAGGAAACTACTGGCTTGGT | SEQ ID NO: 160 | TGGAGAGGAAACTACTGGCTTGGC | SEQ ID NO: 2526 | GAACAATCAGGAATTACTTTTTCA | SEQ ID NO: 4892 |
| rs2857178 | AGTTGGCCATCTTTGCATATGCTA | SEQ ID NO: 161 | AGTTGGCCATCTTTGCATATGCTG | SEQ ID NO: 2527 | TTTCCATTTGAGTTTCTTCTGT | SEQ ID NO: 4893 |
| rs3218822 | AACACAGACAACTCACCACCACCTA | SEQ ID NO: 162 | AACACAGACAACTCACCACCACCTG | SEQ ID NO: 2528 | TATAACAGACTCAGAGCTAGCCTT | SEQ ID NO: 4894 |
| rs3517981 | GTTGCCAAGCACATGTTGCAAA | SEQ ID NO: 163 | GTTGCCAAGCACATGTTGCAAC | SEQ ID NO: 2529 | AAAGAGTCATCTCTGTGTCACC | SEQ ID NO: 4895 |
| rs1610735 | TGCCTCCTGATCTATCGACAGACTT | SEQ ID NO: 164 | TGCCTCCTGATCTATCGACAGACTG | SEQ ID NO: 2530 | GAAGATCAACACTAGGATGATGA | SEQ ID NO: 4896 |
| rs2394737 | AGGCAGGTTGTGCAGAGGATCT | SEQ ID NO: 165 | AGGCAGGTTGTGCAGAGGATCC | SEQ ID NO: 2531 | GTAGGGGAGATAGAGGACAGTT | SEQ ID NO: 4897 |
| rs2239530 | GCAGTAGCCGAAGGTTTTCCTTCTC | SEQ ID NO: 166 | GGAGTAGCCGAAGGTTTTCCTTCTG | SEQ ID NO: 2532 | GCCTTCTGTAACAGTGTAACTTT | SEQ ID NO: 4898 |
| rs4415152 | AATGGTCACATGAAAATGCATGAAT | SEQ ID NO: 167 | AATGGTCACATGAAAATGCATGAAC | SEQ ID NO: 2533 | TGCTAAGAACTTTTCTGCAATAG | SEQ ID NO: 4899 |
| rs4434496 | AACCTTGGACAGAGGCAGAACTCTT | SEQ ID NO: 168 | AACCTTGGACAGAGGCAGAACTCTC | SEQ ID NO: 2534 | TTTCTGTAGTTAAGCCTTCTCAG | SEQ ID NO: 4900 |
| rs3130602 | CCCAGCCCTGTGTAGTTAGAGCAGA | SEQ ID NO: 169 | CCCAGCCCTGTGTAGTTAGAGCAGG | SEQ ID NO: 2535 | ATCAAGATCTCAACACAAATGTG | SEQ ID NO: 4901 |
| rs211456 | GGTGTTGGAGATTTGGGGTACAAAT | SEQ ID NO: 170 | GGTGTTGGAGATTTGGGGTACAAAG | SEQ ID NO: 2536 | CACAGGTTTTGATGATTAGAACA | SEQ ID NO: 4902 |
| rs3091282 | CGATGATGAGCAGAGCTTCCTGG | SEQ ID NO: 171 | CGATGATGAGCAGAGCTTCCTGC | SEQ ID NO: 2537 | TGACTTACTTTGGACGTGTAAC | SEQ ID NO: 4903 |
| rs4959036 | GTGGTTCCATTTCCCCTTGGAT | SEQ ID NO: 172 | GTGGTTCCATTTCCCCTTGGAG | SEQ ID NO: 2538 | GGATACTAATCCCAAAGGAATG | SEQ ID NO: 4904 |
| rs2524103 | CAGGAAGTCTGGAGTCGAGCAAGA | SEQ ID NO: 173 | CAGGAAGTCTGGAGTCGAGCAAGG | SEQ ID NO: 2539 | ATTCTGGATTTATGACAAGGCT | SEQ ID NO: 4905 |
| rs45855 | GTGAGCCACTTTGCCTTTCTGAA | SEQ ID NO: 174 | GTGAGCCACTTTGCCTTTCTGAT | SEQ ID NO: 2S40 | CTCATTTCCTAATCTTTAAGTGG | SEQ ID NO: 4906 |
| rs3130404 | AGATCATGTGGGCATAGGGCTCA | SEQ ID NO: 175 | AGATCATGTGGGCATAGGGCTCG | SEQ ID NO: 2541 | GTATGTGGGAAGGATTTTGATT | SEQ ID NO: 4907 |
| rs211453 | ACCCTCTGGCTCATGTTGGGTA | SEQ ID NO: 176 | ACCCTCTGGCTCATGTTGGGTC | SEQ ID NO: 2542 | TCATCTTGTTTAGTCTGCATCTG | SEQ ID NO: 4908 |
| rs3135375 | TGTTGCCAAATCCAATGGTCATT | SEQ ID NO: 177 | TGTTGCCAAATCCAATGGTCATC | SEQ ID NO: 2543 | CTAGGATGTCATTTTATTCATCAT | SEQ ID NO: 4909 |
| rs3129767 | CCACAATGTACACATCCCAGGATTTT | SEQ ID NO: 178 | CCACAATGTACACATCCCAGGATTTG | SEQ ID NO: 2544 | GGCTGACTTTAAAACTACAAACA | SEQ ID NO: 4910 |
| rs7744593 | GGAATATTGGTGTCCATGGGATTTA | SEQ ID NO: 179 | GGAATATTGGTGTCCATGGGATTTT | SEQ ID NO: 2545 | GCACCATAAGAAATGTAAAAGG | SEQ ID NO: 4911 |
| rs2734979 | GAGTCGTTGAGTGTGAGGAAAGCTA | SEQ ID NO: 180 | GAGTCGTTGAGTGTGAGGAAAGCTG | SEQ ID NO: 2546 | AGAGAAGTAAAGACAGGTGGAGA | SEQ ID NO: 4912 |
| rs3094149 | GCTTGAATCCCATGTTTACACCCAG | SEQ ID NO: 181 | GCTTGAATCCCATGTTTACACCCAC | SEQ ID NO: 2547 | GAAAATACCCAAGTCTGAAGTG | SEQ ID NO: 4913 |
| rs2517723 | TCCCTTTGGGCTTCAGCACAA | SEQ ID NO: 182 | TCCCTTTGGGCTTCAGCACAG | SEQ ID NO: 2548 | GAGATCCCTTACTCTCCAGACTA | SEQ ID NO: 4914 |
| rs1632995 | GATTTCAAGCCCCAGGTAGGAGTA | SEQ ID NO: 183 | GATTTCAAGCCCCAGGTAGGAGTT | SEQ ID NO: 2549 | GCTCATATCTTGCCTAGTTGTG | SEQ ID NO: 4915 |
| rs4248143 | ACCAACTCTACCCCAAGCCCAT | SEQ ID NO: 184 | ACCAACTCTACCCCAAGCCCAC | SEQ ID NO: 2550 | CAGAATTACTAGAACTCCTGGTC | SEQ ID NO: 4916 |
| rs8807 | GGGAAGGTGACAGTGAGGGGT | SEQ ID NO: 185 | GGGAAGGTGACAGTGAGGGGC | SEQ ID NO: 2551 | CTTCAAAGGAGAACAGAGGATAA | SEQ ID NO: 4917 |
| rs3130745 | TTCCATCTGGACCAGTGCCACA | SEQ ID NO: 186 | TTCCATCTGGACCAGTGCCACC | SEQ ID NO: 2552 | CCTTTGTTTGTCTACTTAAACTTG | SEQ ID NO: 4918 |
| rs2858331 | TCTCCAGAGAGTGTGTGGAGCAATAT | SEQ ID NO: 187 | TCTCCAGAGAGTGTGTGGAGCAATAC | SEQ ID NO: 2553 | CCTTTTTTTGGAACTTTACTTCTT | SEQ ID NO: 4919 |
| rs2596503 | TGGGAAGAGGCAGACGATAGATACAT | SEQ ID NO: 188 | TGGGAAGAGGCAGACGATAGATACAC | SEQ ID NO: 2554 | ATAACCAGAGTAAGGAAGGAAAG | SEQ ID NO: 4920 |
| rs2517529 | CCTGTCACTTGTAACCCTGTGTGAG | SEQ ID NO: 189 | CCTGTCACTTGTAACCCTGTGTGAC | SEQ ID NO: 2555 | TGCTTAATCCCTCTAAATCTCAG | SEQ ID NO: 4921 |
| rs761188 | CCCTGTTGAAGCAGTTAGGTGGTAAT | SEQ ID NO: 190 | CCCTGTTGAAGCAGTTAGGTGGTAAC | SEQ ID NO: 2556 | GTTTGGTGTAACAAAAAATCTTTT | SEQ ID NO: 4922 |
| rs2295664 | GTCAGAGTAATGGTGAGGGGCAGT | SEQ ID NO: 191 | GTCAGAGTAATGGTGAGGGGCAGG | SEQ ID NO: 2557 | GTGACGTTATTTCATTACACGG | SEQ ID NO: 4923 |
| rs2523504 | CCGTCTTTGAGATGGGGAGCT | SEQ ID NO: 192 | CCGTCTTTGAGATGGGGAGCC | SEQ ID NO: 2558 | CAAAAGTAAGACCTGATGTCCT | SEQ ID NO: 4924 |
| rs7381625 | TGTTTACATTCAACTGCACTGACCAA | SEQ ID NO: 193 | TGTTTACATTCAACTGCACTGACCAG | SEQ ID NO: 2559 | CACAGATTCTAAGAGATGCTGAC | SEQ ID NO: 4925 |
| rs407161 | TTTGCCCTCAAGATTCTTTCTTTGT | SEQ ID NO: 194 | TTTGCCCTCAAGATTCTTTCTTTGC | SEQ ID NO: 2560 | TCTTGGCAGTTTGATTATAATGT | SEQ ID NO: 4926 |
| rs3900268 | AACTGGAATGAGTCAGGCTGGGA | SEQ ID NO: 195 | AACTGGAATGAGTCAGGCTGGGG | SEQ ID NO: 2561 | AAGTAATCAGGATGAGTCAGGG | SEQ ID NO: 4927 |
| rs3117123 | ACCCCCAGGACATCATTAAATGTA | SEQ ID NO: 196 | ACCCCCAGGACATCATTAAATGTG | SEQ ID NO: 2562 | TTACTCTTCCAATCCAAAGTATTC | SEQ ID NO: 4928 |
| rs211455 | GGTGGTTTGGACTGTTATTCCCAATT | SEQ ID NO: 197 | GGTGGTTTGGACTGTTATTCCCAATC | SEQ ID NO: 2563 | TTTGGTTCCTCCTTATTATTTAAG | SEQ ID NO: 4929 |
| rs7451341 | CAAAGGCTTGCTGCAATCTGGA | SEQ ID NO: 198 | CAAAGGCTTGCTGCAATCTGGG | SEQ ID NO: 2564 | AGTGAAGTTCAAGAATAATAGCTG | SEQ ID NO: 4930 |
| rs3095312 | GGCAGCCAGGAGGACTGTGAG | SEQ ID NO: 199 | GGCAGCCAGGAGGACTGTGAC | SEQ ID NO: 2565 | AAGAAAACAAGACAAACAAGCT | SEQ ID NO: 4931 |
| rs6932266 | CGGTGTTTCCACAGTATCCATCACT | SEQ ID NO: 200 | CGGTGTTTCCACAGTATCCATCACC | SEQ ID NO: 2566 | AAATCACATGCATTGTATCCAT | SEQ ID NO: 4932 |
| rs2295120 | CCCTCTGAGCACCAAAGTCAAAA | SEQ ID NO: 201 | CCCTCTGAGCACCAAAGTCAAAG | SEQ ID NO: 2567 | TGTGAATAAACTCTGGTAGAGGC | SEQ ID NO: 4933 |
| rs458679 | TCATCTCATGGTCCTCTTCGTGTG | SEQ ID NO: 202 | TCATCTCATGGTCCTCTTCGTGTC | SEQ ID NO: 2568 | CTCTCATGATTTGTCATTGGAT | SEQ ID NO: 4934 |
| rs763028 | TTTTGCATCCTCTGATTCAGTCAAT | SEQ ID NO: 203 | TTTTGCATCCTCTGATTCAGTCAAC | SEQ ID NO: 2569 | ATGGATTGAAAATATTTGGAAAA | SEQ ID NO: 4935 |
| rs1360 | CCCCCAAACAATTCCACCATGT | SEQ ID NO: 204 | CCCCCAAACAATTCCACCATGC | SEQ ID NO: 2570 | TACTTATATAGCACTCACCAAACC | SEQ ID NO: 4936 |
| rs2734574 | AGAAGGGAGTGGCTGGTCCCT | SEQ ID NO: 205 | AGAAGGGAGTGGCTGGTCCCA | SEQ ID NO: 2571 | TTTTGATCATCTAGGAACAGGA | SEQ ID NO: 4937 |
| rs1548515 | CTTGCCAGGAGGAACCGTCAA | SEQ ID NO: 206 | CTTGCCAGGAGGAACCGTCAC | SEQ ID NO: 2572 | AGGACACACACTCATAAAGCTT | SEQ ID NO: 4938 |
| rs1610736 | TCATCATGACCATTCTGCTCACCA | SEQ ID NO: 207 | TCATCATGACCATTCTGCTCACCG | SEQ ID NO: 2573 | CATCATCCTAGTGTTGATCTTCC | SEQ ID NO: 4939 |
| rs1573648 | CTCATCTGCCTCTGGGCAGACA | SEQ ID NO: 208 | CTCATCTGCCTCTGGGCAGACG | SEQ ID NO: 2574 | TTTTCTGTGAAGATTCTCACTCC | SEQ ID NO: 4940 |
| rs3807035 | GCCTGTTTTCTTTGAGCATGTCCA | SEQ ID NO: 209 | GCCTGTTTTCTTTGAGCATGTCCG | SEQ ID NO: 2575 | CTGTTCCTAAATAATACTGGACATT | SEQ ID NO: 4941 |
| rs3763354 | ATTTGATGCCTTGGCCATCGACT | SEQ ID NO: 210 | ATTTGATGCCTTGGCCATCGACC | SEQ ID NO: 2576 | GAAAAAAGTCACTGAAGTGAACTT | SEQ ID NO: 4942 |
| rs2855437 | CAGGAGGGCCCTGGGTAAGAA | SEQ ID NO: 211 | CAGGAGGGCCCTGGGTAAGAG | SEQ ID NO: 2577 | AGAGAGTCAGAGACACCAAAAC | SEQ ID NO: 4943 |
| rs3117008 | GGAATACACCTTGCATGACAGGAAAT | SEQ ID NO: 212 | GGAATACACCTTGCATGACAGGAAAC | SEQ ID NO: 2578 | GAGAAACAGTCACTCTCTCAATTC | SEQ ID NO: 4944 |
| rs3823342 | CCCAACACACATTAGGTCCTCCAA | SEQ ID NO: 213 | CCCAACACACATTTAGGTCCTCCAG | SEQ ID NO: 2579 | CTCTCACCTTTACAAGCTGTGA | SEQ ID NO: 4945 |
| rs2254618 | TTGAGGGGTGCATAAACGTAAGACT | SEQ ID NO: 214 | TTGAGGGGTGGATTAACGTAAGACA | SEQ ID NO: 2580 | GTTGATGCCTGATATAGATTGG | SEQ ID NO: 4946 |
| rs1541270 | CACAGAGCTCCTAAAACCCTTGAAA | SEQ ID NO: 215 | CACAGAGCTCCTAAAACCCTTGAAC | SEQ ID NO: 2581 | GATGATCCTAAACTAATGATGGG | SEQ ID NO: 4947 |
| rs3817962 | GGGAAACTCAAAGGGGCTCAGA | SEQ ID NO: 216 | GGGAAACTCAAAGGGGCTCAGC | SEQ ID NO: 2582 | ATCGGCTGGTTATGTTTTAAAC | SEQ ID NO: 4948 |
| rs3763313 | TTACAGTTTCCCCCTTTCCAAGAAA | SEQ ID NO: 217 | TTACAGTTTCCCCCTTTCCAAGAAC | SEQ ID NO: 2583 | TACTGCTAGTAAGATTTTATGGGA | SEQ ID NO: 4949 |
| rs1611651 | CAGCCCACTGAACTACACATAATGGT | SEQ ID NO: 218 | CAGCCCACTGAACTACACATTATGGC | SEQ ID NO: 2584 | GTCACGAGAGAAGTTTTGATAGTT | SEQ ID NO: 4950 |
| rs2516713 | TGAGTTTTGGTTCTCCCAACCTTCT | SEQ ID NO: 219 | TGAGTTTTGGTTCTCCCAACCTTCA | SEQ ID NO: 2585 | CCTGGATACTTGTGACATAATCC | SEQ ID NO: 4951 |
| rs3869070 | CCTTTCTCAAACTCATGTTTTTGGCA | SEQ ID NO: 220 | CCTTTCTCAAACTCATGTTTTTGGCG | SEQ ID NO: 2586 | TCATTGATAAATCAGAAAGCAAA | SEQ ID NO: 4952 |
| rs7743412 | CATGTGCTGGGTTGTCCAGCA | SEQ ID NO: 221 | CATGTGCTGGGTTGTCCAGCG | SEQ ID NO: 2587 | GTCCTCAGTCTCTGTGTGTAGC | SEQ ID NO: 4953 |
| rs1541267 | GCTGGAACATCAAATGCCCACT | SEQ ID NO: 222 | GCTGGAACATCAAATGCCCACC | SEQ ID NO: 2588 | CATTGTCTAAGTTCCCTCCAGT | SEQ ID NO: 4954 |
| rs2853977 | GATGGTCCAGTCCCTGACCCT | SEQ ID NO: 223 | GATGGTCCAGTCCCTGACCCA | SEQ ID NO: 2589 | TGTCTTTATCCAGTGGCTCTAA | SEQ ID NO: 4955 |
| rs3891156 | AGCATCTCCACCCTCCCCTAGAA | SEQ ID NO: 224 | AGCATCTCCACCCTCCCCTAGAG | SEQ ID NO: 2590 | TCTCAGAATGTCCAGTGACTGT | SEQ ID NO: 4956 |
| rs2394607 | TGCAATCACTATCAAAATCCCAAAA | SEQ ID NO: 22S | TGCAATCACTATCAAAATCCCAAAG | SEQ ID NO: 2591 | CATTTTTTACAGAAATGGAAAAA | SEQ ID NO: 4957 |
| rs1028411 | AACCACTCGTATCTCTCCACCCTT | SEQ ID NO: 226 | AACCACTCGTATCTCTCCACCCTG | SEQ ID NO: 2592 | GTCATGAAAGATGATGACTTTAAA | SEQ ID NO: 4958 |
| rs2022065 | GGTCTTCTGACTTCAAATCCAGTGCT | SEQ ID NO: 227 | GGTCTTCTGACTTCAAATCCAGTGCC | SEQ ID NO: 2593 | TTTCTATGCAGCTACTTCTGTG | SEQ ID NO: 4959 |
| rs2294748 | CCTGCAGTGAGTAGAGCCAAGTTTT | SEQ ID NO: 228 | CCTGCAGTGAGTAGAGCCAAGTTTC | SEQ ID NO: 2594 | TGACTTTTTCTGACCACATTAAT | SEQ ID NO: 4960 |
| rs6933541 | CGCCCCTGCCCATTTTTAGT | SEQ ID NO: 229 | CGCCCCTGCCCATTTTTAGC | SEQ ID NO: 2595 | CTTTGGTCGTATACTAAGAACCTC | SEQ ID NO: 4961 |
| rs1419638 | GGAACACAGAAATGGAATGGAGGAAA | SEQ ID NO: 230 | GGAACACAGAAATGGAATGGAGGTAG | SEQ ID NO: 2596 | AAGGTAGTACAGGAGAGGAAAAT | SEQ ID NO: 4962 |
| rs13395 | GCACCTGCATGTGTCTGTGTTCT | SEQ ID NO: 231 | GCACCTGCATGTGTCTGTGTTCA | SEQ ID NO: 2597 | GTAGGCATAATGTGAGGAGGT | SEQ ID NO: 4963 |
| rs64036 | GAGGGATTGTCTGAAGAGAAGCTGT | SEQ ID NO: 232 | GAGGGATTGTCTGAAGAGAAGCTGG | SEQ ID NO: 2598 | TATCTGGCTTCTGTGTTCTTTT | SEQ ID NO: 4964 |
| rs727939 | GGTTGTCCTCTAGAACACCCACACA | SEQ ID NO: 233 | GGTTGTCCTCTAGAACACCCACACG | SEQ ID NO: 2599 | TTTCCTCCAAGTTATATGCTGA | SEQ ID NO: 4965 |
| rs10751 | CCAGGAATGAGGTCCCCCAAA | SEQ ID NO: 234 | CCAGGAATGAGGTCCCCCAAG | SEQ ID NO: 2600 | TGCTAAGTTTTACATAGGGGAG | SEQ ID NO: 4966 |
| rs3869065 | AGCGTGGTCTTGGATGGCAGG | SEQ ID NO: 235 | AGCGTGGTCTTGGATGGCAGC | SEQ ID NO: 2601 | AGACAGGTAGGTTTCTGCACTTA | SEQ ID NO: 4967 |
| rs3094061 | TGAACAAACCAACATTCAGGCTCA | SEQ ID NO: 236 | TGAACAAACCAACATTCAGGCTCC | SEQ ID NO: 2602 | TTGGAGTATGCTTTTAGGCTTT | SEQ ID NO: 4968 |
| rs419434 | TCTCCAACTATGTGGCTTGCCCA | SEQ ID NO: 237 | TCTCCAACTATGTGGCTTGCCCG | SEQ ID NO: 2603 | CTACCAGACCCTTCTTGAGAAAT | SEQ ID NO: 4969 |
| rs429916 | CAGGTGTCTCCTAGGCGCCTATT | SEQ ID NO: 238 | CAGGTGTCTCCTAGGCGCCTATG | SEQ ID NO: 2604 | TGTGCTAGGCATTACGTAGAC | SEQ ID NO: 4970 |
| rs3130603 | ATGCAAATTCCCACATCTGAAAGT | SEQ ID NO: 239 | ATGCAAATTCCCACATCTGAAAGG | SEQ ID NO: 2605 | TTTGGAAGAAGAATTCTAATCAGA | SEQ ID NO: 4971 |
| rs397144 | CCCAGGGTAACCTACCTCCCTCA | SEQ ID NO: 240 | CCCAGGGTAACCTACCTCCCTCG | SEQ ID NO: 2606 | CATATCGTCTGACAGGTGTGT | SEQ ID NO: 4972 |
| rs3873332 | TTTTTCCCTCCCCTGCATTCAT | SEQ ID NO: 241 | TTTTTCCCTCCCCTGCATTCAC | SEQ ID NO: 2607 | ACATTCAAACATAGTATATCGTTCA | SEQ ID NO: 4973 |
| rs2735052 | GCATTGACTTCATTGCTCAGCTAAAT | SEQ ID NO: 242 | GCATTGACTTCATTGCTCAGCTAAAC | SEQ ID NO: 2608 | CTGTAACCAAAGGATGAGTCAT | SEQ ID NO: 4974 |
| rs3134759 | GACCCTGGTTGGGAGGTCTTGTA | SEQ ID NO: 243 | GACCCTGGTTGGGAGGTCTTGTC | SEQ ID NO: 2609 | AGTCACGAGGAATAGGATCAG | SEQ ID NO: 4975 |
| rs2571430 | TGCACCTGTGCTTTTATTTCAGGA | SEQ ID NO: 244 | TGCACCTGTGCTTTTATTTCAGGG | SEQ ID NO: 2610 | CTGAGACAATTGTATTTATCAGATG | SEQ ID NO: 4976 |
| rs4713367 | GAGATGGGGATCCCTGATTTGTAGT | SEQ ID NO: 245 | GAGATGGGGATCCCTGATTTGTAGC | SEQ ID NO: 2611 | GTTATCCGTGTGTAGTTATTCCG | SEQ ID NO: 4977 |
| rs2523774 | GGAAGCCAGTCACAGGAGGCTACA | SEQ ID NO: 246 | GGAAGCCAGTCACAGGAGGCTACG | SEQ ID NO: 2612 | AACTTATGAGCCCATTTTTGT | SEQ ID NO: 4978 |
| rs2233969 | ACAGGCTGGGCTAGTGGTTGTGA | SEQ ID NO: 247 | ACAGGCTGGGCTAGTGGTTGTGG | SEQ ID NO: 2613 | GAGAATAAACCTCCACATTCC | SEQ ID NO: 4979 |
| rs522162 | GGAACAGGCACTGGCCATGTT | SEQ ID NO: 248 | GGAACAGGCACTGGCCATGTC | SEQ ID NO: 2614 | TTACACTGAGATCAAACCTGAC | SEQ ID NO: 4980 |
| rs3763324 | TCATTTGAGGATTCAGGTCAATGAAT | SEQ ID NO: 249 | TCATTTGAGGATTCAGGTCAATGAAC | SEQ ID NO: 2615 | TGAGAGAGTATAATGGGCAAAG | SEQ ID NO: 4981 |
| rs1033568 | GGAGAACGAAGACATCTTGGCAT | SEQ ID NO: 250 | GGAGAACGPAGACATCTTGGCAC | SEQ ID NO: 2616 | TTCATCTGCTTGGTACAGATC | SEQ ID NO: 4982 |
| rs1632984 | TGAGGAAAGGACTCTTGGCTGAGA | SEQ ID NO: 251 | TGAGGAAAGGACTCTTGGCTGAGT | SEQ ID NO: 2617 | GATCCTGGATACCTTGAGAATG | SEQ ID NO: 4983 |
| rs1362060 | TTGACAAGGCATGTCCATAAGACTCA | SEQ ID NO: 252 | TTGACAAGGTATGTCCATAAGACTCG | SEQ ID NO: 2618 | AGTGACCTCTGTGTCTAACATAGA | SEQ ID NO: 4984 |
| rs7754443 | CAGACAGTGCTTCAGAGGGCACT | SEQ ID NO: 253 | CAGACAGTGCTTCAGAGGGCACC | SEQ ID NO: 2619 | TGCTCCACTTGGTCTTAATTT | SEQ ID NO: 4985 |
| rs2395351 | TGGTGATAGTGGCATCGATGACTA | SEQ ID NO: 254 | TGGTGATAGTGGCATCGATGACTG | SEQ ID NO: 2620 | AGTTTTGGTAAATCTCTAAAAATTG | SEQ ID NO: 4986 |
| rs2267644 | GAATGCCAGTCAGAATGCAAAATTA | SEQ ID NO: 255 | GAATGCCAGTCAGAATGCAAAATTG | SEQ ID NO: 2621 | AGTCAATAAAATCACAAAAGAGAA | SEQ ID NO: 4987 |
| rs29232 | ATCCCCCACCACCCATGAGT | SEQ ID NO: 256 | ATCCCCCACCACCCATGAGC | SEQ ID NO: 2622 | TTTTGATAGCATTGCAAGTATAAT | SEQ ID NO: 4988 |
| rs1633085 | AGGGAATCTGGTGTCCTTAGATCACT | SEQ ID NO: 257 | AGGGAATCTGGTGTCCTTAGATCACC | SEQ ID NO: 2623 | GAGTTAATAGTAGCAGGGAAGGA | SEQ ID NO: 4989 |
| rs7743506 | TCAAAGTTTAGGGGAACATCGAGATA | SEQ ID NO: 258 | TCAAAGTTTAGGGGAACATCGAGATC | SEQ ID NO: 2624 | GATTTTTCTAGTTGAAGCATAAGC | SEQ ID NO: 4990 |
| rs1737009 | CACAGACCTTTCAAGGACTCACCTT | SEQ ID NO: 259 | CACAGACCTTTCAAGGACTCACCTA | SEQ ID NO: 2625 | TTAGGACAGTCAAAGCAGGATA | SEQ ID NO: 4991 |
| rs2844606 | GCACATCAGTGATGAATGCAAGAGA | SEQ ID NO: 260 | GCACATCAGTGATGAATGCAAGAGG | SEQ ID NO: 2626 | GTTTCTATAAGAACTGGCATAAATC | SEQ ID NO: 4992 |
| rs2394173 | AAAGGGGAGCTGGTATGTGCAA | SEQ ID NO: 261 | AAAGGGGAGCTGGTATGTGCAG | SEQ ID NO: 2627 | AGTCACGTGGTAAGAGAGGAAC | SEQ ID NO: 4993 |
| rs4392759 | GCCAAACAAGTCTATGAGGTCACCTA | SEQ ID NO: 262 | GCCAAACAAGTCTATGAGGTCACCTG | SEQ ID NO: 2628 | CAAGGAAGACCTTATCAAAGAG | SEQ 10 NO: 4994 |
| rs3132528 | TTAAAACTAGGTAGGAAAGGGCGGA | SEQ ID NO: 263 | TTAAATCTAGGTAGGAAAGGGCGGG | SEQ ID NO: 2629 | GTGGCAGGTTTAAATAGAACAG | SEQ ID NO: 4995 |
| rs3130782 | TGCTCCATAAGGCTCTTTAGAAGCA | SEQ ID NO: 264 | TGCTCCATAAGGCTCTTTAGAAGCG | SEQ ID NO: 2630 | TCAACTTCCCCTATTTTACATCT | SEQ ID NO: 4996 |
| rs379221 | TTGTGAGCCAACAAATCTATGGCTA | SEQ ID NO: 265 | TTGTGAGCCAACPAATCTATGGCTG | SEQ ID NO: 2631 | GGAAACAGTCAACTCCATTTCT | SEQ ID NO: 4997 |
| rs7195 | GGGTGGCTATAGGGCTGGAAAAT | SEQ ID NO: 266 | GGGTGGCTATAGGGCTGGAAAAC | SEQ ID NO: 2632 | CTGAAGATGACTGCTTTCACT | SEQ ID NO: 4998 |
| rs7383433 | TCCTTGAAAACCAGAGTTCTTGGTA | SEQ ID NO: 267 | TCCTTGAAAACCAGAGTTCTTGGTG | SEQ ID NO: 2633 | AAAAGAAAGGAGATGCAGAAGTA | SEQ ID NO: 4999 |
| rs1632854 | AGTGGAGCTGACTGGTCCGAGAA | SEQ ID NO: 268 | AGTGGAGCTGACTGGTCCGAGAT | SEQ ID NO: 2634 | ATTTTTTTAAATGCCTCCTTG | SEQ ID NO: 5000 |
| rs5007259 | GCATTTCCCTAAAGGACATTGATTCT | SEQ ID NO: 269 | GCATTTCCCTAAAGGACATTGATTCC | SEQ ID NO: 2635 | TCCGTATGTCGTACTCTGTAATC | SEQ ID NO: 5001 |
| rs3130377 | AACAGGCAAAGGACTTCAAGACTA | SEQ ID NO: 270 | TTTCAGGCAAAGGACTTCAAGACTG | SEQ ID NO: 2636 | GGAATTCTTACAAGTTTTCCTTAG | SEQ ID NO: 5002 |
| rs1419632 | AAAGCATCACCATGGCCCAAA | SEQ ID NO: 271 | AAAGCATCACCATGGCCCAAC | SEQ ID NO: 2637 | GTTACAATGAGCATACAGAAGC | SEQ ID NO: 5003 |
| rs3619721 | TTCCCTTCACTTGTTTTCCTTTCTT | SEQ ID NO: 272 | TTCCCTTCACTTGCTTTCCTTTCTC | SEQ ID NO: 2638 | TTCTAGACATACTCAAACAAACAAA | SEQ ID NO: 5004 |
| rs2071363 | CAGATATTTTGTCACCTGCAGCAACA | SEQ ID NO: 273 | CAGATATTTTGTCACCTGCAGCAACG | SEQ ID NO: 2639 | ACACAGTTATTGTCATTCCTGG | SEQ ID NO: 5005 |
| rs2233966 | TCTCATAAGGATGGCATCCAGGT | SEQ ID NO: 274 | TCTCATAAGGATGGCATCCAGGC | SEQ ID NO: 2640 | CATTTCCCTAGCTAGATCCAG | SEQ ID NO: 5006 |
| rs2621416 | TTCCATATTGGTGTCATGACAATCAA | SEQ ID NO: 275 | TTCCATATTGGTGTCATGACAATCAG | SEQ ID NO: 2641 | TATATTTCAAAAACTTGTTTCCG | SEQ ID NO: 5007 |
| rs1736912 | TCTGTCCTTAGCCAGGGACCTTCT | SEQ ID NO: 276 | TCTGTCCTTAGCCAGGGACCTTCC | SEQ ID NO: 2642 | CTGACCTGTGATTACTGGGAT | SEQ ID NO: 5008 |
| rs3763347 | GAAATCTCCCCTCACTGCCAAAA | SEQ ID NO: 277 | GAAATCTCCCCTCACTGCCAAAT | SEQ ID NO: 2643 | AGCAGTGGCAAGTAGGAAGTAG | SEQ ID NO: 5009 |
| rs1610613 | CAGGGCAGCTGTGGTCCCTA | SEQ ID NO: 278 | CAGGGCAGCTGTGGTCCCTG | SEQ ID NO: 2644 | TTTCATCATATGTCCAAGTGTTAC | SEQ ID NO: 5010 |
| rs3135373 | CCATTCTTCTTCCACTCTTCACAGAT | SEQ ID NO: 279 | CCATTCTTCTTCCACTCTTCACAGAC | SEQ ID NO: 2645 | AGGCTACAGTCCACATCTTTAAC | SEQ ID NO: 5011 |
| rs2523946 | AGCAGCCACACTCCAAAAGAGGGT | SEQ ID NO: 280 | AGCAGTCACACTCCAAAAGAGGGC | SEQ ID NO: 2646 | CAGGTTAGAAAACCCTCACAG | SEQ ID NO: 5012 |
| rs3873374 | CCTAGACTGGGCTTTTAAACACTGGAA | SEQ ID NO: 281 | CCTAGACTGGGCTTTTTAACACTGGAG | SEQ ID NO: 2647 | GTGGATGTGATTTCTAGTTTCAG | SEQ ID NO: 5013 |
| rs17034 | AGTGTCCAAATCTCCATCGTGCT | SEQ ID NO: 282 | AGTGTCCAAATCTCCATCGTGCC | SEQ ID NO: 2648 | GCAACTCAGGAACAGCTATCT | SEQ ID NO: 5014 |
| rs2073149 | TCATTGCTGGTTATTTATGCCTCAGT | SEQ ID NO: 283 | TCATTGCTGGTTAAATGCCTCAGA | SEQ ID NO: 2649 | GAGTTCTCTGGTTAATTCAAATCA | SEQ ID NO: 5015 |
| rs2073150 | AACGCAGGATGGATATAGGTGAAA | SEQ ID NO: 284 | AACGCAGGATGGATATAGGTGAAG | SEQ ID NO: 2650 | GAACAGGTGCATAGAAAAGAATA | SEQ ID NO: 5016 |
| rs3117007 | GACATCTTCACGCACAGAAGGAGG | SEQ ID NO: 285 | GACATCTTCACGCACAGAAGGAGC | SEQ ID NO: 2651 | AGAAAGGTGAGAAATCCTGTG | SEQ ID NO: 5017 |
| rs3094207 | CCCCACCTTTTGCCTATGGAAGG | SEQ ID NO: 286 | CCCCACCTTTTGCCTATGGAAGC | SEQ ID NO: 2652 | ATGTGCTTTGAGCTTGATTTT | SEQ ID NO: 5018 |
| rs3748082 | CCTAGTTAAGTGGCAAGATGCCCAT | SEQ ID NO: 287 | CCTAGTAAAGTGGCAAGATGCCCAC | SEQ ID NO: 2653 | ACTTGTGGAAATGGGAATCTTA | SEQ ID NO: 5019 |
| rs423808 | GGCTGACTTCAGGGGTGGAGTAAA | SEQ ID NO: 288 | GGCTGACTTCAGGGGTGGAGTAAC | SEQ ID NO: 2654 | GGAGTCTCTCAGTTCCTTCTC | SEQ ID NO: 5020 |
| rs445150 | TGTGGATAAGGGGAACCACTGTATAT | SEQ ID NO: 289 | TGTGGATAAGGGGAACCACTGTATAC | SEQ ID NO: 2655 | CAGATCTTTGTGAAGAATACTGC | SEQ ID NO: 5021 |
| rs3734841 | TGGGTTTTTGTTGATCTGAGGAACTT | SEQ ID NO: 290 | TGGGTTTTTGTTGATCTGAGGAACTC | SEQ ID NO: 2656 | TACCAACCTTACTCATCAGTGC | SEQ ID NO: 5022 |
| rs3132523 | CCAAAACAGAATTTGGAAAATCCAA | SEQ ID NO: 291 | CCAAAACAGAATTTGGAAAATCCAG | SEQ ID NO: 2657 | ATAGCAGAGGGCATTTATCAGT | SEQ ID NO: 5023 |
| rs3997854 | TGCTGATGGAAAGACAACTTTGTTT | SEQ ID NO: 292 | TGCTGATGGAAAGACAACTTTGTTG | SEQ ID NO: 2658 | TATTGTTGTGGCTTGTATGTCTT | SEQ ID NO: 5024 |
| rs2071482 | GCCCAGCACCATCTGTGTGATT | SEQ ID NO: 293 | GCCCAGCACCATCTGTGTGATG | SEQ ID NO: 2659 | CCTTCCATTCTTAACCCTTCT | SEQ ID NO: 5025 |
| rs2071469 | AATCGTCAGCCTCTTCAGAATGAGT | SEQ ID NO: 294 | AATCGTCAGCCTCTTCAGAATGAGC | SEQ ID NO: 2660 | CATAAAATTCAGTCAGAAAGTACC | SEQ ID NO: 5026 |
| rs2736155 | CCTAGGGGACAGGCATGTGTTAAAC | SEQ ID NO: 295 | CCTAGGGGACAGGCATGTGTTAAAG | SEQ ID NO: 2661 | GTGAGTCACAGTGAGAGGTCAG | SEQ ID NO: 5027 |
| rs2524198 | GCTCCCGTGTCGGTGTCTCT | SEQ ID NO: 296 | GCTCCCGTGTCGGTGTCTCC | SEQ ID NO: 2662 | AATCCTGGAGTACAGATGTTTC | SEQ ID NO: 5028 |
| rs560505 | CATGGATGAAGAACTTGCAAAAAAAT | SEQ ID NO: 297 | CATGGATGAAGAACTTGCAAAAAAAC | SEQ ID NO: 2663 | TTGTTCCAAAATCCAGATTCTA | SEQ ID NO: 5029 |
| rs2894252 | AAGAAAATGTCTATGGCAAAGGGCT | SEQ ID NO: 298 | AAGAAAATGTCTATGGCAAAGGGCC | SEQ ID NO: 2664 | AAGATTAGAATCTACTACGCTCAGC | SEQ ID NO: 5030 |
| rs3094220 | TCTGAGAGTCTGGGTGGTGCTCT | SEQ ID NO: 299 | TCTGAGAGTCTGGGTGGTGCTCC | SEQ ID NO: 2665 | CCTACGCCTCTAGAGGTCTCT | SEQ ID NO: 5031 |
| rs3763355 | TGTTAAGAATGAACCCAGGGTATCAT | SEQ ID NO: 300 | TGTTAAGAATGAACCCAGGGTATCC | SEQ ID NO: 2666 | GTAACCACAAGTTCACTTCAGTG | SEQ ID NO: 5032 |
| rs3823312 | GAGCATCCAGTGGCACACTGTAGA | SEQ ID NO: 301 | GAGCATCCAGTGGCAGACTGTAGG | SEQ ID NO: 2667 | GTTTAGTAACTGCTGGCATTCC | SEQ ID NO: 5033 |
| rs4999342 | GTAAGAACCATGGGTGTGAACCATT | SEQ ID NO: 302 | GTAAGAACCATGGGTGTGAACCATC | SEQ ID NO: 2668 | GAAAAAATCTGTGTTGAACAAG | SEQ ID NO: 5034 |
| rs2517403 | CTCATGGAGTAACAAAACCAACACCA | SEQ ID NO: 303 | CTCATGGAGTTACAAAACCAACACCG | SEQ ID NO: 2669 | AATCAATTTCAGAACATTTTCATC | SEQ ID NO: 5035 |
| rs4987086 | TCCAGGTATGGAATACAGGGGACA | SEQ ID NO: 304 | TCCAGGTATGGAATACAGGGGACG | SEQ ID NO: 2670 | TTAAGAAGATATGGCCACACA | SEQ ID NO: 5036 |
| rs3819294 | GGGACTGGGAGGAAGCAGGA | SEQ ID NO: 305 | GGGACTGGGAGGAAGCAGGG | SEQ ID NO: 2671 | CATGAGATCTTAGAGGAACCTC | SEQ ID NO: 5037 |
| rs909728 | TCATGGACTGTTCATCGTGTAGTGA | SEQ ID NO: 306 | TCATGGACTGTTCATCGTGTAGTGG | SEQ ID NO: 2672 | GGATGGTACAAGATGGTAGATG | SEQ ID NO: 5038 |
| rs2294477 | GGAGATGGTCTGGCATCTGTGAG | SEQ ID NO: 307 | GGAGATGGTCTGGCATCTGTGAC | SEQ ID NO: 2673 | GTTTATTCACACTGTGAGGGG | SEQ ID NO: 5039 |
| rs3117229 | CAAGCAGGAAAGAGAATGACTCTGAT | SEQ ID NO: 308 | CAAGCAGGAAAGAGAATGACTCTGAC | SEQ ID NO: 2674 | GGATATGGTTTGTAATTTGTAATG | SEQ ID NO: 5040 |
| rs3909178 | TTAAGGTTGCTATTCGGCTGACTTT | SEQ ID NO: 309 | TTAAGGTTGCTATTCGGCTGACTTC | SEQ ID NO: 2675 | CAGAGATTATCATGGATTATTCAG | SEQ ID NO: 5041 |
| rs3116985 | AGGGTAACCTCCTTTTGTTGGAACA | SEQ ID NO: 310 | AGGGTTACCTCCTTTTGTTGGAACG | SEQ ID NO: 2676 | TCTGTTTATAGGAGAAAAAAACAA | SEQ ID NO: 5042 |
| rs3095320 | TCCTGAGCCCCAGTTTCTGAAAT | SEQ ID NO: 311 | TCCTGAGCCCCAGTTTCTGAAAC | SEQ ID NO: 2677 | CTGAAGTGGGGTTAATGACAC | SEQ ID NO: 5043 |
| rs241445 | CCCAAGAAGGCACAGACTGTTTCT | SEQ ID NO: 312 | CCCAAGAAGGCACAGACTGTTTCC | SEQ ID NO: 2678 | CTAGTAGGTCCTTCGTCCTCC | SEQ ID NO: 5044 |
| rs3a28896 | CCTGGAGGGTTCATAGTCTAATGGGA | SEQ ID NO: 313 | CCTGGAGGGTTCATAGTCTAATGGGG | SEQ ID NO: 2679 | AGGAGGGTAGAAGATGGTAGGT | SEQ ID NO: 5045 |
| rs6914995 | CGAGCTATGGACTCTGCATTAGGAGAC | SEQ ID NO: 314 | CGAGCTATGGACTCTGCATTAGGAGAG | SEQ ID NO: 2680 | AAGTGCAAAGAGTGTTTCTGTAT | SEQ ID NO: 5046 |
| rs3815086 | CTCTGCTGCCTGGTCCTCACTA | SEQ ID NO: 315 | CTCTGCTGCCTGGTCCTCACTG | SEQ ID NO: 2681 | GATGAGTGACAAGATAGAGCCC | SEQ ID NO: 5047 |
| rs3132708 | GGGAGACCAGGGACTCCTCTGATA | SEQ ID NO: 316 | GGGAGACCAGGGACTCCTCTGATG | SEQ ID NO: 2682 | CCAACTGTAGCTCAAGGACTC | SEQ ID NO: 5048 |
| rs6457644 | GATGTGAGATCTGAGACTGAACCCAT | SEQ ID NO: 317 | GATGTGAGATCTGAGACTGAACCCAC | SEQ ID NO: 2683 | ACTTCCCTTCTAAGATACAATTCTG | SEQ ID NO: 5049 |
| rs6914849 | AGAGCCCTAGGGTCCTAGAAGAGAGA | SEQ ID NO: 318 | AGAGCCCTAGGGTCCTAGAAGAGAGG | SEQ ID NO: 2684 | AGGATACAGAAACACTCTTTGC | SEQ ID NO: 5050 |
| rs4151655 | GACAGGACCATGAGGGTAGGAGATA | SEQ ID NO: 319 | GACAGGACCATGAGGGTAGGAGATT | SEQ ID NO: 2685 | GGAAGATAAACTGGCTAAAGG | SEQ ID NO: 5051 |
| rs6457327 | TGAGTCTCTCTGTCCACTGTGACATTT | SEQ ID NO: 320 | TGAGTGTCTCTGTCCACTGTGACATTG | SEQ ID NO: 2686 | CATATGATGAATTCAGGAGAACT | SEQ ID NO: 5052 |
| rs2647087 | GCCTCAAGGGAAGCCCCAGT | SEQ ID NO: 321 | GCCTCAAGGGAAGCCCCAGG | SEQ ID NO: 2687 | GTGCAGAATCACATGACAATACT | SEQ ID NO: 5053 |
| rs3830060 | CCTGCTGTGTCTTTGGAGAAATTCA | SEQ ID NO: 322 | CCTGCTGTGTCTTTGGAGTAATTCG | SEQ ID NO: 2688 | ATCTTCAAAAATAACCCCATG | SEQ ID NO: 5054 |
| rs915653 | TCTATCCCCATCCTCTCCAGATAGTT | SEQ ID NO: 323 | TCTATCCCCATCCTCTCCAGATAGTC | SEQ ID NO: 2689 | CCTAACCAAGTGAAGTAGGTTAAG | SEQ ID NO: 5055 |
| rs2524059 | CAGACACCCCTTGTCTCTTGGGTG | SEQ ID NO: 324 | CAGACACCCCTTGTCTCTTGGGTC | SEQ ID NO: 2690 | GGACACTGGGTAAAGTGGAATA | SEQ ID NO: 5056 |
| rs6912673 | TGATAAAAGGCACCTACAAAAACCCTA | SEQ ID NO: 325 | TGATAAAAGGCACCTACAAAAACCCTG | SEQ ID NO: 2691 | AGTTGATGTTTACTGGACGTTATT | SEQ ID NO: 5057 |
| rs4713218 | TGGGGAGAAACATTTGACATCTTTT | SEQ ID NO: 326 | TGGGGAGAAACATTTGACATCTC | SEQ ID NO: 2692 | ATGTCTTTTGCATTTTGAATCA | SEQ ID NO: 5058 |
| rs3135372 | TTGGATTTAGGGAAAAGGAAATGCA | SEQ ID NO: 327 | TTGGATTTAGGGAAAAGGAAATGCC | SEQ ID NO: 2693 | TGGCATGTTAATATGCTGATT | SEQ ID NO: 5059 |
| rs3807034 | ATGGAAAGGCGAGAGGCACCA | SEQ ID NO: 328 | ATGGAAAGGCGAGAGGCACCG | SEQ ID NO: 2694 | GAATGAAAACCACACATCAAC | SEQ ID NO: 5060 |
| rs2158285 | TCTCACCGGTACAAACCCTAAAAGTT | SEQ ID NO: 329 | TCTCACCGGTACAAACCCTAAAAGTC | SEQ ID NO: 2695 | GTTTTCAGGAACAAATAGCTCTG | SEQ ID NO: 5061 |
| rs2735061 | GTGGGCTCTGACCAGGTCCTA | SEQ ID NO: 330 | GTGGGCTCTGACCAGGTCCTG | SEQ ID NO: 2696 | TTTTGTTCTACCCCAATCACT | SEQ ID NO: 5062 |
| rs204995 | CACACAGGGTTCCCAGGGCTT | SEQ ID NO: 331 | CACACAGGGTTCCCAGGGCTC | SEQ ID NO: 2697 | CTCTGTTTTCTGTACTCTGTCTCTC | SEQ ID NO: 5063 |
| rs2596501 | ACATTTGTCTGTGAAATTGGACCCA | SEQ ID NO: 332 | ACAAAGTCTGTGAAATTGGACCCG | SEQ ID NO: 2698 | CATCAAGGACTGTTCTTTCCT | SEQ ID NO: 5064 |
| rs2256902 | ACATATGCAAAGGGACAAGAATGAA | SEQ ID NO: 333 | ACATATGCAAAGGGACAAGAATGAG | SEQ ID NO: 2699 | ATTTCAGATACAAGGAAAACTGG | SEQ ID NO: 5065 |
| rs2844586 | GGTAAACTTTTGAGCACCCTGGCTA | SEQ ID NO: 334 | GGTTAACTTTTGAGCACCCTGGCTG | SEQ ID NO: 2700 | GGGTGTCCATTAAAATGATTG | SEQ ID NO: 5066 |
| rs2293861 | GAGGCTTCCCAATAAGCTGCAGA | SEQ ID NO: 335 | GAGGCTTCCCAATAAGCTGCAGG | SEQ ID NO: 2701 | TATTAAAGGGTAGGGAGCAGG | SEQ ID NO: 5067 |
| rs1233408 | GCACAGATGCAAGTAGGTGAACAGA | SEQ ID NO: 336 | GCACAGATGCAAGTAGGTGAACAGG | SEQ ID NO: 2702 | GGGTAAGTAAGAACTTCTCTTTTT | SEQ ID NO: 5068 |
| rs7382794 | GAGTCTCCCTAGTGTAACCTTGACCCA | SEQ ID NO: 337 | GAGTCTCCCTAGTGTAACCTTGACCCG | SEQ ID NO: 2703 | AATAACAGCAGTGTCTTTATCCC | SEQ ID NO: 5069 |
| rs3830061 | GAGTCAGTGCAGGAGCCCTGA | SEQ ID NO: 338 | GAGTCAGTGCAGGAGCCCTGG | SEQ ID NO: 2704 | GAAGAGAGAAGAGGGATGATCA | SEQ ID NO: 5070 |
| rs2524074 | AGGCCCATTGCACAGGACAGTT | SEQ ID NO: 339 | AGGGCCATTGCACAGGACAGTC | SEQ ID NO: 2705 | TACTTTCCCACTTGAAAAGTG | SEQ ID NO: 5071 |
| rs3131025 | ACAAACAGGCATTTTGCACTATTCTT | SEQ ID NO: 340 | ACAAACAGGCATTTTGCACTATTCTC | SEQ ID NO: 2706 | CTTCTCTACCAGTCAGTATGGTG | SEQ ID NO: 5072 |
| rs1034324 | GTCCTAACTCCAAGGCAAGGGTT | SEQ ID NO: 341 | GTCCTTACTCCAAGGCAAGGGTC | SEQ ID NO: 2707 | TCACTCATAGTGCTTCACCAC | SEQ ID NO: 5073 |
| rs3130655 | AGAGGGAGGGGCCAGGAGGTT | SEQ ID NO: 342 | AGAGGGAGGGGCCAGGAGGTA | SEQ ID NO: 2708 | CATTGCACTAGAATTACTGTGAGA | SEQ ID NO: 5074 |
| rs4713324 | CATTGGTCAGGAAAAGAGAAACAGAGA | SEQ ID NO: 343 | CATTGGTCAGGAAAAGAGAAACAGAGG | SEQ ID NO: 2709 | AGAAAAACTGAGCAAATAACATG | SEQ ID NO: 5075 |
| rs3129888 | TGAGTTAGGTGTGGTCAGAGGAAGACA | SEQ ID NO: 344 | TGAGTTAGGTGTGGTCAGAGGAAGACG | SEQ ID NO: 2710 | ATATGGAGATATCTGAGGGAGG | SEQ ID NO: 5076 |
| rs2071366 | CCCACCCTATCCTGTAGTTAGGGAAA | SEQ ID NO: 345 | CCCACCCTATCCTGTAGTTAGGGAAG | SEQ ID NO: 2711 | ACCCAGGACTGAATCAATATC | SEQ ID NO: 5077 |
| rs2074479 | TCTGTGTAGATTTGAAGTCCCAACAT | SEQ ID NO: 346 | TCTGTGTAGATTTGAAGTCCCAACAC | SEQ ID NO: 2712 | CAAGTCATCTAATTCAGAGGATG | SEQ ID NO: 5078 |
| rs2516312 | TGACTGTCCAGCATCATCTCTGAGTA | SEQ ID NO: 347 | TGACTGTCCAGCATCATCTCTGAGTG | SEQ ID NO: 2713 | GGGGACCAAAAAATTTATCTT | SEQ ID NO: 5079 |
| rs1737078 | GCTCCTTCGTGCCTAGCAATGTT | SEQ ID NO: 348 | GCTCCTTCGTGCCTAGCAATGTG | SEQ ID NO: 2714 | TAGCTTTACCCCAGCCTTAAC | SEQ ID NO: 5080 |
| rs3734854 | GCTCCACCCTCAAGCACTTGGA | SEQ ID NO: 349 | GCTCCACCCTCAAGCACTTGGG | SEQ ID NO: 2715 | ACTGATCCTTTGGGAAGGATG | SEQ ID NO: 5081 |
| rs6901541 | GAGATCGATCGAAGACTGTGAAAGTT | SEQ ID NO: 350 | GAGATCGATCGAAGACTGTGAAAGTC | SEQ ID NO: 2716 | GCTGTTCATAGAACAACTCTTTTT | SEQ ID NO: 5082 |
| rs3916766 | CAGCTTTGTTGTTGAAGGATGTTTTA | SEQ ID NO: 351 | CAGCAAGTTGTTGAAGGATGTTTTT | SEQ ID NO: 2717 | TGATTTTAGAATTCTAGGTTTGTG | SEQ ID NO: 5083 |
| rs6919972 | TGGGAACACACTGGCCCATTATATAA | SEQ ID NO: 352 | TGGGAACACACTGGCCCATTATATAG | SEQ ID NO: 2718 | GAAAAATAAAACATGATCCCC | SEQ ID NO: 5084 |
| rs6911639 | AATCATCGATTTCATGGTCTTTGACA | SEQ ID NO: 353 | AATCATCGATTTCATGGTCTTTGACG | SEQ ID NO: 2719 | ATGGCCTAATTTGGTTAGATTT | SEQ ID NO: 5085 |
| rs6921314 | TCCTGCACACTTCAGCACAAAACT | SEQ ID NO: 354 | TCCTGCACACTACAGCACAAAACC | SEQ ID NO: 2720 | TATATTTCTCTTCAGGAACAATAACC | SEQ ID NO: 5086 |
| rs3135351 | TCGGGTTTTTCATTGAAAGTCATT | SEQ ID NO: 355 | TCGGGTTTTTCATTGAAAGTCATG | SEQ ID NO: 2721 | AGGACTATGCTCTAGAAATGGG | SEQ ID NO: 5087 |
| rs3129299 | TCACAGCAGTGGGAATCAAAGTGA | SEQ ID NO: 356 | TCACAGCAGTGGGAATCAAAGTGG | SEQ ID NO: 2722 | AGGATCCCAGAGAAAGGAAGTA | SEQ ID NO: 5088 |
| rs7745235 | CCTCCACAAGACCCACCCTGA | SEQ ID NO: 357 | CCTCCACAAGACCCACCCTGG | SEQ ID NO: 2723 | GCACAATATTGCTTCTGGTTT | SEQ ID NO: 5089 |
| rs3819285 | CTGGGACCCTGTGTGCCAGA | SEQ ID NO: 358 | CTGGGACCCTGTGTGCCAGC | SEQ ID NO: 2724 | CTTACTCTTTTGTGCAGCACAT | SEQ ID NO: 5090 |
| rs1233493 | CTCCAGTAGGTCATTCAGATCTCCAT | SEQ ID NO: 359 | CTCCAGTAGGTCATTCAGATCTCCAC | SEQ ID NO: 2725 | TCCTACTAAGAAACTCCCCTCAT | SEQ ID NO: 5091 |
| rs2066744 | GCTGCAGAACAAATTCGAAAGTAAAAA | SEQ ID NO: 360 | GCTGCAGAACAAATTCGAAAGTTAAAG | SEQ ID NO: 2726 | CGTTCAAGTCTGAATCTTCATT | SEQ ID NO: 5092 |
| rs1367728 | CTAGAAAGGGGTTGCAGGGTTGT | SEQ ID NO: 361 | CTAGAAAGGGGTTGCAGGGTTGC | SEQ ID NO: 2727 | GGAGATGCAGCTCCTAAAAAAT | SEQ ID NO: 5093 |
| rs2853960 | GCAGTGCTCCAAGACAAGAAATTAATT | SEQ ID NO: 362 | GCAGTGCTCCAAGACAAGAAATTTATC | SEQ ID NO: 2728 | AGTATCTGGTAGGGATTCAGAGT | SEQ ID NO: 5094 |
| rs4151672 | GGTTTCCTGCTGGACAGGGGT | SEQ ID NO: 363 | GGTTTCCTGCTGGACAGGGGC | SEQ ID NO: 2729 | TGGGATTGAATTAAAACAGCT | SEQ ID NO: 5095 |
| rs3691157 | GCTCAGGAGAGAGAAGGGAGGAATTA | SEQ ID NO: 364 | GCTCAGGAGAGAGAAGGGAGGAAAC | SEQ ID NO: 2730 | CTGAAGATTTTGTGTTATTCATG | SEQ ID NO: 5096 |
| rs2394255 | CGACTGGGCTGCAGCCCTAT | SEQ ID NO: 365 | CGACTGGGCTGCAGCCCTAC | SEQ ID NO: 2731 | TACAGGTGTCCTTCTTTATACCTG | SEQ ID NO: 5097 |
| rs3128967 | CAGAGTCATTCTCTTTCCTGCTTGTCT | SEQ ID NO: 366 | CAGAGTCATTCTCTTTCGTGCTTGTCG | SEQ ID NO: 2732 | CTGTACTCATGTTACAGGTTAGGG | SEQ ID NO: 5098 |
| rs3130106 | GCAGCCATTCACACTATACATTTTTGT | SEQ ID NO: 367 | GCAGCCATTCACACTATACATTTTTGC | SEQ ID NO: 2733 | AGTAGCCTTCAAAACATTTTCTT | SEQ ID NO: 5099 |
| rs1059231 | GCTGGATGAGGTCATCTCCAAATAT | SEQ ID NO: 368 | GGTGGATGAGGTCATCTCCAAATAC | SEQ ID NO: 2734 | CAATGTTGCAAGACAAAAGTG | SEQ ID NO: 5100 |
| rs356966 | CCTGCTACGTGACTGTTCTCTCCAT | SEQ ID NO: 369 | CCTGCTACGTGACTGTTCTCTCCAC | SEQ ID NO: 2735 | GTGGTCCTGCTAATGTGAGTC | SEQ ID NO: 5101 |
| rs2394605 | TTTCTCTGACTTGGGCTGACAATTAC | SEQ ID NO: 370 | TTTCTCTGACTTGGGCTGACAATTAG | SEQ ID NO: 2736 | GTACTGTGAGACAACACCCTTG | SEQ ID NO: 5102 |
| rs2295118 | TCTGTGCGTCTGCACAAACTCT | SEQ ID NO: 371 | TCTGTGCGTCTGCACAAACTCC | SEQ ID NO: 2737 | CATATGTTGACACATGGTCTG | SEQ ID NO: 5103 |
| rs2050191 | GCCAAATCAACTTTTGGAGATTTTCTA | SEQ ID NO: 372 | GCCAAATCAACTTTTGGAGATTTTCTT | SEQ ID NO: 2738 | TAATGTACCTCTAAAATGAACAGC | SEQ ID NO: 5104 |
| rs427417 | TTTCCTCTCATACGGGAGCTTTCTT | SEQ ID NO: 373 | TTTCCTCTCATACGGGAGCTTTCTC | SEQ ID NO: 2739 | ATTCTGCTCATCTTGTTCCTGT | SEQ ID NO: 5105 |
| rs3828808 | CCCCACCTCAGTGTCCCTCAGA | SEQ ID NO: 374 | CCCCACCTCAGTGTCCCTCAGG | SEQ ID NO: 2740 | TAGAGGCCTCTAATCCATCCT | SEQ ID NO: 5106 |
| rs7383005 | TTAAAAAAGCTGGGCTGATCTTACAA | SEQ ID NO: 375 | TTTAAAAAGCTGGGCTGATCTTACAG | SEQ ID NO: 2741 | GCTTGCAAAGGTTTAGTAAAAA | SEQ ID NO: 5107 |
| rs6940585 | CTGTGGCAGAACAGAGCTGCCA | SEQ ID NO: 376 | CTGTGGCAGAACAGAGCTGCCG | SEQ ID NO: 2742 | TGGTCTCTCCATCTTCACACT | SEQ ID NO: 5108 |
| rs2294745 | TGTGAGGGGTAGGTTTTCCTTCCT | SEQ ID NO: 377 | TGTGAGGGGTAGGTTTTCCTTCCC | SEQ ID NO: 2743 | AAACCTGAGTATATAGAGGATTTAGG | SEQ ID NO: 5109 |
| rs3128854 | CCTTTGGGCAGTGATTAGGTCAT | SEQ ID NO: 378 | CCTTTGGGCAGTGATTAGGTCAC | SEQ ID NO: 2744 | AGGGAAGAGGCTTCATAAATG | SEQ ID NO: 5110 |
| rs1633037 | CAATCTGATGTGCAAGGTAATTCCATT | SEQ ID NO: 379 | CAATCTGATGTGCAAGGTAATTCCATC | SEQ ID NO: 2745 | TGCAAAGGAAGATTTGTTAC | SEQ ID NO: 5111 |
| rs3132550 | CTTCGTGAGAGCCCAGGCTGA | SEQ ID NO: 380 | CTTCGTGAGAGCCCAGGCTGG | SEQ ID NO: 2746 | GTCAGGAATGGAAACCCTATT | SEQ ID NO: 5112 |
| rs1610708 | CTGGTACCTGCGCATTGCAGA | SEQ ID NO: 381 | CTGGTACCTGCGCATTGCAGG | SEQ ID NO: 2747 | TCTCCTCATTCTTAAGGCCTT | SEQ ID NO: 5113 |
| rs2844766 | CTTGCCATGAGGTTTGGGTTGATT | SEQ ID NO: 382 | CTTGCCATGAGGTTGGGTTGATC | SEQ ID NO: 2748 | TrCAATGAAAGGAGAGCAAGA | SEQ ID NO: 5114 |
| rs4713447 | TCAACAGGAATTTCCTTCAGGATAAA | SEQ ID NO: 383 | TCAACAGGAATTTCCTTCAGGATAAG | SEQ ID NO: 2749 | AGGAAAAGAACATTTAGGCTTTT | SEQ ID NO: 5115 |
| rs4151671 | GGTGGGAGCAGCTGAAGTGAT | SEQ ID NO: 384 | GGTGGGAGCAGCTGAAGTGAC | SEQ ID NO: 2750 | CAGTCTATTCGTCCAGAGGAA | SEQ ID NO: 5116 |
| rs733840 | AATCACAACATCGTGCCACAGTA | SEQ ID NO: 385 | AATCACAACATCGTGCCACAGTT | SEQ ID NO: 2751 | CTATTATCTAATTGGGAAGATTTTC | SEQ ID NO: 5117 |
| rs3198005 | CCAGGAAGTCTGCATAATCACGAA | SEQ ID NO: 386 | CCAGGAAGTCTGCATTATCACGAG | SEQ ID NO: 2752 | AGCTTGGAAAGGAGGTAACAC | SEQ ID NO: 5118 |
| rs2523814 | GGTAATCTCCCAAGGCCCTGCT | SEQ ID NO: 387 | GGTAATCTCCCAAGGCCCTGCA | SEQ ID NO: 2753 | CTGTGCTCTGACATCCTCAGTA | SEQ ID NO: 5119 |
| rs7755580 | CAATGGCAGTGGCTAGAAAGAGTAAA | SEQ ID NO: 388 | CAATGGCAGTGGCTAGAAAGAGTAAT | SEQ ID NO: 2754 | AGGCCACGTACAGTGTCATATAT | SEQ ID NO: 5120 |
| rs2071364 | AAGGCTCAATGAAAGGATAATCTCCA | SEQ ID NO: 389 | AAGGCTCAATGAAAGGATAATCTCCG | SEQ ID NO: 2755 | ATTAGTGCTACCAAAGTGTCATT | SEQ ID NO: 5121 |
| rs2894239 | GAACAAAAACTGGATGAGAAGCCTT | SEQ ID NO: 390 | GAACAAAAACTGGATGAGATTTCCCTG | SEQ ID NO: 2756 | TCTTGTTTTATGTGGTTGAGAG | SEQ ID NO: 5122 |
| rs3117230 | GATTTCCAGGAAGGAGGAAGTAGAGT | SEQ ID NO: 391 | GATTTCCAGGAAGGAGGAAGTAGAGC | SEQ ID NO: 2757 | GAGTCAGAAGTTCAACAGAAGTC | SEQ ID NO: 5123 |
| rs1628578 | CAGCCCATGGTTCTGCATCTCT | SEQ ID NO: 392 | CAGCCCATGGTTCTGCATCTCG | SEQ ID NO: 2758 | GGAAGTCAGGATAAATTTGGC | SEQ ID NO: 5124 |
| rs362531 | CAGGGCCACATTGGGACACTT | SEQ ID NO: 393 | CAGGGCCACATTGGGACACTC | SEQ ID NO: 2759 | TAGATAACCGCCACAATTGTT | SEQ ID NO: 5125 |
| rs6899851 | GTTCACCTTTTCCCCATAGGTTAGTA | SEQ ID NO: 394 | GTTCACCTTTTCCCCATAGGTTAGTG | SEQ ID NO: 2760 | TGTCTCCTTTGCTATACTGAAAG | SEQ ID NO: 5126 |
| rs4084091 | AATAGCCTTGGCAGTAAACGCCA | SEQ ID NO: 395 | AATAGCCTTGGCAGTAAACGCCG | SEQ ID NO: 2761 | GAGTTCAGAAGAGTCACGGTG | SEQ ID NO: 5127 |
| rs2735097 | CCCAAAGGGAAACATCCCTCATA | SEQ ID NO: 396 | CCCAAAGGGAAACATCCCTCATC | SEQ ID NO: 2762 | AAGCCAAACCAGAAGCAATAT | SEQ ID NO: 5128 |
| rs1264698 | AAAGAATGCCACGGATTGCCA | SEQ ID NO: 397 | AAAGAATGCCACGGATTGCCC | SEQ ID NO: 2763 | CAAACACCAGGAGCTAGAAGA | SEQ ID NO: 5129 |
| rs2071789 | CCTTCCTGTACGGGGCCCTA | SEQ ID NO: 398 | CCTTCCTGTACGGGGCCCTG | SEQ ID NO: 2764 | ATTTCTCATCTCATTTCTGGG | SEQ ID NO: 5130 |
| rs3093544 | AGCTCCTCCATGTGCCTGCT | SEQ ID NO: 399 | AGCTCCTCCATGTGCCTGCC | SEQ ID NO: 2765 | TTCCTCTGTGTGTGGATCTAG | SEQ ID NO: 5131 |
| rs3132130 | TGAGAGCCCAGAACATGCATATAAAGG | SEQ ID NO: 400 | TGAGAGCCCAGAACATGCATATAAAGC | SEQ ID NO: 2766 | AACATATCTGTAAACTTCAAGGAA | SEQ ID NO: 5132 |
| rs3093669 | CCACCACGTCCGGCAGTTT | SEQ ID NO: 401 | CCACCACGTCCGGCAGTTC | SEQ ID NO: 2767 | GTTCCTTGTTCTTAATTGTCCTC | SEQ ID NO: 5133 |
| rs2092305 | TGTGTTCAAATAAGGATACACCCAGCT | SEQ ID NO: 402 | TGTGTTCAAATAAGGATACACCCAGCC | SEQ ID NO: 2768 | TAACCAATTGGGTGTTTCTGTA | SEQ ID NO: 5134 |
| rs3128927 | TGAGAAAGTGCCCTAGAACCTGTACA | SEQ ID NO: 403 | TGAGAAAGTGCCCTAGAACCTGTACG | SEQ ID NO: 2769 | TCTAAACTGTGAAACCCTGTTC | SEQ ID NO: 5135 |
| rs1633009 | TGTGAGTTCTCTGTACGGGGAAGTTT | SEQ ID NO: 404 | TGTGAGTTCTCTGTACGGGGAAGTTA | SEQ ID NO: 2770 | AAAAAGCAGGTCAAACTGTGAT | SEQ ID NO: 5136 |
| rs6914678 | TGTGAGTTTCTCTGTACGGGGAAGTTT | SEQ ID NO: 405 | TGTGAGTTCTCTGTACGGGGAAGTTA | SEQ ID NO: 2771 | AAAAAGCAGGTCAAACTGTGAT | SEQ ID NO: 5137 |
| rs8337 | GGGCAACTTACTCTTCCTGGCATG | SEQ ID NO: 406 | GGGCAACTTACTCTTCCTGGCATC | SEQ ID NO: 2772 | TATTGTATTGGAGGGTGACCA | SEQ ID NO: 5138 |
| rs2286712 | CCAGGGTAAGTGGCACCACAGG | SEQ ID NO: 407 | CAGGGTAAGTGGCACCACAGC | SEQ ID NO: 2773 | AGGAACAGAGGGTGTGAGAAT | SEQ ID NO: 5139 |
| rs1633086 | CCCCTATGCCTCCCACTCCT | SEQ ID NO: 408 | CCCCTATGCCTCCCACTCCC | SEQ ID NO: 2774 | ACTCTCCCTGACCCTAACTCT | SEQ ID NO: 5140 |
| rs2245514 | CCTGCGGTCCCCAGGTTCC | SEQ ID NO: 409 | CCTGCGGTCCCCAGGTTCG | SEQ ID NO: 2775 | GGTTTTTCACGTCTAATCTCAAT | SEQ ID NO: 5141 |
| rs1265086 | TTTAGGTCCCATCTGCTCGCTCT | SEQ ID NO: 410 | TTTAGGTCCCATCTGCTCGCTCG | SEQ ID NO: 2776 | TCTCCCACTAGGATGTAGGCT | SEQ ID NO: 5142 |
| rs466384 | CCTTCATAGCTGGACTCCATAAAGACA | SEQ ID NO: 411 | CCTTCATAGCTGGACTCCATAAAGACG | SEQ ID NO: 2777 | AGTACCTGGAAGAGAAGAAGAAC | SEQ ID NO: 5143 |
| rs3117225 | TCCAAATGTCCCCTAGTTCACAATAA | SEQ ID NO: 412 | TCCAAATGTCCCCTAGTTCACAATAG | SEQ ID NO: 2778 | AAGGGCTTAAATAGAGAAGTAAAG | SEQ ID NO: 5144 |
| rs209473 | CAGCTATTGCCAGCCACCGA | SEQ ID NO: 413 | CAGCTATTGCCAGCCACCGC | SEQ ID NO: 2779 | ACTGTTTAGAGGCTGAAGCAG | SEQ ID NO: 5145 |
| rs4151664 | CCTGGGCCACTGAAGCAAAAGA | SEQ ID NO: 414 | CCTGGGCCACTGAAGCAAAAGG | SEQ ID NO: 2780 | CACACACTTTGAGAAAGAGGG | SEQ ID NO: 5146 |
| rs909967 | CCTTGATTCAGAGCTCAGATGGATGA | SEQ ID NO: 415 | CCTTGATTCAGAGCTCAGATGGATGG | SEQ ID NO: 2781 | GCCTTAACTGTTACCTAGCCC | SEQ ID NO: 5147 |
| rs1633013 | TCACACTGCAAATCTCTATGCTCAGA | SEQ ID NO: 416 | TCACACTGCAAATCTCTATGCTCAGG | SEQ ID NO: 2782 | AGAACACAGACCTTTCAAGGAC | SEQ ID NO: 5148 |
| rs4151657 | TTTCCACATGTGTCAAATGTAGGACTA | SEQ ID NO: 417 | TTTCCACATGTGTCAAATGTAGGACTG | SEQ ID NO: 2783 | AGAAAGATCTCTGAAGTGCTCA | SEQ ID NO: 5149 |
| rs1419689 | GCTGGAAATACAGGTACACCTGGAAAT | SEQ ID NO: 418 | GCTGGAAATACAGGTACACCTGGAAAG | SEQ ID NO: 2784 | ACTTAGAAATAAATGCTTGTTGATT | SEQ ID NO: 5150 |
| rs3757333 | CCAGCGGGACCCAAGGAAA | SEQ ID NO: 419 | CCAGCGGGACCCAAGGAAC | SEQ ID NO: 2785 | CTAGAACTATACGTCCCAGAACA | SEQ ID NO: 5151 |
| rs1264541 | TGTGTTAAAAGGCACATATGTGCTTTT | SEQ ID NO: 420 | TGTGTTTAAAGGCACATATGTGCTTTG | SEQ ID NO: 2786 | TGCATGTAATTACTGCACATACA | SEQ ID NO: 5152 |
| rs7992 | CCATCCCCACTGTCCTCAAGAGA | SEQ ID NO: 421 | CCATCCCGACTGTCCTCAAGAGG | SEQ ID NO: 2787 | ACCAGGAAGGACTAGGCTACA | SEQ ID NO: 5153 |
| rs2523926 | AGCACTCTGGAGAAAGCCGGGT | SEQ ID NO: 422 | AGCACTCTGGAGAAAGCCGGGC | SEQ ID NO: 2788 | TGTGTCCTGATGCTCTTATCAC | SEQ ID NO: 5154 |
| rs3823419 | ACAAGGTTCATGGCCTGGGAAA | SEQ ID NO: 423 | ACAAGGTTCATGGCCTGGGAAG | SEQ ID NO: 2789 | GAAGACAGACATGAATAAATCATT | SEQ ID NO: 5155 |
| rs2735003 | TCTGAGTCCCTGAGAACCTAGGGT | SEQ ID NO: 424 | CTGAGTCCCTGAGAACCTAGGGG | SEQ ID NO: 2790 | TTGGTATGGGTTCACTGAAAAT | SEQ ID NO: 5156 |
| rs3817965 | CTGAGGAGAGACACACACGTGTGTAT | SEQ ID NO: 425 | CTGAGGAGAGACACACACCTGTGTAC | SEQ ID NO: 2791 | TTACCCTTCTGAGATCAGGAAG | SEQ ID NO: 5157 |
| rs2076540 | CAATTTTCCAAACCTGAAGTGTTGGA | SEQ ID NO: 426 | CAATTTCCAAACCTGAAGTGTTGGG | SEQ ID NO: 2792 | AAACAGAGTAAATAAAGGATAAGAGC | SEQ ID NO: 5158 |
| rs3998799 | TGAACTTGTCCTGAGAATGAAGGTG | SEQ ID NO: 427 | TGAACTTGTCCTGAGAATGAAGGTC | SEQ ID NO: 2793 | AGAGACAGTTTCTTCTTCTTCCT | SEQ ID NO: 5159 |
| rs2428510 | TAAGAAATCTTTGTGAAGCTGCCAT | SEQ ID NO: 428 | TAAGAAATCTTTGTGAAGCTGCCAC | SEQ ID NO: 2794 | CAGGTTTGTCAATTTGTGACAT | SEQ ID NO: 5160 |
| rs3094187 | TTGGAGGCTCCCCAAATATTTTGT | SEQ ID NO: 429 | TTGGAGGCTCCCCAAATATTTTGC | SEQ ID NO: 2795 | ATCTGAGGATCCAGCTCAAGT | SEQ ID NO: 5161 |
| rs2076484 | CCTGTGCAGGACCAGGTTCTTTT | SEQ ID NO: 430 | CCTGTGCAGGACCAGGTTCTTTC | SEQ ID NO: 2796 | CTGGGCTCCAAGATCTTAAAG | SEQ ID NO: 5162 |
| rs3763349 | CACCTGGGGACTCCTGTGTGT | SEQ ID NO: 431 | CACCTGGGGACTCCTGTGTGC | SEQ ID NO: 2797 | CCATTGTTAATGGTTTGAGGA | SEQ ID NO: 5163 |
| rs2523512 | AACATCCCCCACAGCTGTCAGA | SEQ ID NO: 432 | AACATCCCCCACAGCTGTCAGG | SEQ ID NO: 2798 | TGTCAAGGGTAACAGAGGTCAT | SEQ ID NO: 5164 |
| rs2517439 | CAGGGCCCAGAGTGCTTAGAGA | SEQ ID NO: 433 | CAGGGCCCAGAGTGCTTAGAGG | SEQ ID NO: 2799 | CATCATCTCTCCTTTGCTCTG | SEQ ID NO: 5165 |
| rs1419634 | CAAGGAAGTGAATGACTTGTACACTGGA | SEQ ID NO: 434 | CAAGGAAGTGAATGACTTGTACACTGGC | SEQ ID NO: 2800 | ACTGTAAAAACATTGATGAAAGG | SEQ ID NO: 5166 |
| rs241438 | TGGTGAAGGTCGTGTCCCTGTAA | SEQ ID NO: 435 | TGGTGAAGGTCGTGTCCCTGTAG | SEQ ID NO: 2801 | TTGATGTTTGCTGTTCCTCT | SEQ ID NO: 5167 |
| rs2244839 | GACAGACCATGGCCATGGCA | SEQ ID NO: 436 | GACAGACCATGGCCATGGCG | SEQ ID NO: 2802 | GATGATAGCAGCTCTCCTACG | SEQ ID NO: 5168 |
| rs3135033 | ATCACCTATTCCCAACGCGTG | SEQ ID NO: 437 | ATCACCTATTGCCCAACGCGTC | SEQ ID NO: 2803 | CTATTAAGTACTTACGAATCCTCAA | SEQ ID NO: 5169 |
| rs3135388 | CCTCATCAGGAAAAACCTAAAGTGGGA | SEQ ID NO: 438 | CCTCATCAGGAAAACCTAAAGTGGGG | SEQ ID NO: 2804 | TGGTTTGTTGGGAGATCTCTA | SEQ ID NO: 5170 |
| rs3807033 | CAGATCCTGGTTTGTGTGGCTTTA | SEQ ID NO: 439 | CAGATCCTGGTTTGTGTGGCTTTG | SEQ ID NO: 2805 | CAAGCCTCTGAGTGTTGATGT | SEQ ID NO: 5171 |
| rs4713446 | TGGCTTGAAGATTAGGGAAATAGTGTT | SEQ ID NO: 440 | TGGCTTGAAGATTAGGGAAATAGTGTC | SEQ ID NO: 2806 | ACATTTCAAAAGAGAAGACTGCT | SEQ ID NO: 5172 |
| rs3115608 | CCAGTTCAGGGACGGAGATTCCT | SEQ ID NO: 441 | CCAGTTCAGGGACGGAGATTCCG | SEQ ID NO: 2807 | GATGAAAAGTGAAGGGAGAGG | SEQ ID NO: 5173 |
| rs1736994 | CAATCCTGCTAATGTGAGTCAGATTGTT | SEQ ID NO: 442 | CAATCCTGCTAATGTGAGTCAGATTGTG | SEQ ID NO: 2808 | CATTTCTTCACTTAAAAGCCTCT | SEQ ID NO: 5174 |
| rs6913309 | AGGATACTGGAAGGGAACTCACTCTA | SEQ ID NO: 443 | AGGATACTGGAAGGGAACTCACTCTT | SEQ ID NO: 2809 | CTGGGCTTTAGAATTATTTCTAGT | SEQ ID NO: 5175 |
| rs6905408 | ATTGGTCCCTGAGCTTAGCCCTA | SEQ ID NO: 444 | ATTGGTCCCTGAGCTTAGCCCTG | SEQ ID NO: 2810 | AGGAAAAATGGCTTTTTTTC | SEQ ID NO: 5176 |
| rs3128917 | CTGCCCAGCAAGAAACTCAGATAA | SEQ ID NO: 445 | CTGCCCAGCAAGAAACTCAGATTC | SEQ ID NO: 2811 | ACAGGTTCCACTATCTGACCC | SEQ ID NO: 5177 |
| rs6934041 | AGCTCTGCCGTTTGACCTGTGTC | SEQ ID NO: 446 | AGCTCTGCCGTTTGACCTGTGTG | SEQ ID NO: 2812 | TCCACAAGAAAGAAACTTCC | SEQ ID NO: 5178 |
| rs6916248 | TTCTGGAAATTGAAGGAAGGAAACT | SEQ ID NO: 447 | TTCTGGAAATTGAAGGAAGGAAACC | SEQ ID NO: 2813 | GACCTCCTTCTAAACACTCGAG | SEQ ID NO: 5179 |
| rs5007263 | CTTCCTCACTCAGCCTGCTTCACA | SEQ ID NO: 448 | CTTCCTCACTCAGCCTGCTTCACG | SEQ ID NO: 2814 | GAACACTGTGAGGGTCAAAT | SEQ ID NO: 5180 |
| rs3819300 | CCCTGGGCACTGTCGCTGG | SEQ ID NO: 449 | CCCTGGGCACTGTCGCTGC | SEQ ID NO: 2815 | TGGAGTAGAACAAAAACAGGAC | SEQ ID NO: 5181 |
| rs2071471 | CCTCCATGACTCAAGGCACAGAT | SEQ ID NO: 450 | CCTCCATGACTCAAGGCACAGAC | SEQ ID NO: 2816 | CTCCAGGTAAGAACAGAGCAAT | SEQ ID NO: 5182 |
| rs2894179 | AAAATGGAAATGGCCTAAATGTCCA | SEQ ID NO: 451 | AAAATGGAAATGGCCTAAATGTCCC | SEQ ID NO: 2817 | CAACGGATGAATGGAAAATAA | SEQ ID NO: 5183 |
| rs2239705 | ATGCCTGGACCCTTGTCTGTTTT | SEQ ID NO: 452 | ATGCCTGGACCCTTGTCTGTTTC | SEQ ID NO: 2818 | GCTTTTCCCTTTCTCTCTTC | SEQ ID NO: 5184 |
| rs2301220 | CCCAGACTTTCACATACACATTCATCA | SEQ ID NO: 453 | CCCAGACTTTCACATACACATTCATCG | SEQ ID NO: 2819 | TTTCTTTCATGATAATAGTTGCTT | SEQ ID NO: 5185 |
| rs2272904 | GGAGAATGGACCCCCAGGGA | SEQ ID NO: 454 | GGAGAATGGACCCCCAGGGC | SEQ ID NO: 2820 | ACTTGGAAAGCGAGTAAGTGAG | SEQ ID NO: 5186 |
| rs6935418 | TGCTAGACTCTCCATAGGATGCTGAATA | SEQ ID NO: 455 | TGCTAGACTCTCCATAGGATGCTGAATC | SEQ ID NO: 2821 | AAGTAGTGAGAGCTGACATCCTC | SEQ ID NO: 5187 |
| rs5021450 | TGAAAAAAGGTAAGTTTCCCTGTGATT | SEQ ID NO: 456 | TGAAAAAAGGTAAGTTTCCCTGTGATG | SEQ ID NO: 2822 | CTACATTTTAGCTCATTCCCTAGT | SEQ ID NO: 5188 |
| rs2857103 | CCTCTGCTTCCCTTTTGTTTGCA | SEQ ID NO: 457 | CCTCTGCTTCCCTTTTGTTTGCC | SEQ ID NO: 2823 | TCTCTTGTTATGTCTTTGCCC | SEQ ID NO: 5189 |
| rs2853953 | CACAGACACCTTCCTGCCCTAGTA | SEQ ID NO: 458 | CACAGACACCTTCCTGCCCTAGTG | SEQ ID NO: 2824 | ATTTGGACTGGAGTTTCCTT | SEQ ID NO: S190 |
| rs757259 | GAAGAAGCAATTCACTGACTCCTTTCTT | SEQ ID NO: 459 | GAAGAAGCAATTCACTGACTCCACTC | SEQ ID NO: 2825 | AACTGGGGATAAATAACCTCTAA | SEQ ID NO: 5191 |
| rs3135391 | TCTCTCAGTTCCACAGGGCTGTTT | SEQ ID NO: 460 | TCTCTCAGTTCCACAGGGCTGTTC | SEQ ID NO: 2826 | TGAGCACAGTTACCTCTGGAG | SEQ ID NO: 5192 |
| rs2523606 | CCTCACCCCTTCCCTACCTGAA | SEQ ID NO: 461 | CCTCACCCCTTCCCTACCTGAG | SEQ ID NO: 2827 | TCTTCCTCCTACACATCACAG | SEQ ID NO: 5193 |
| rs1632962 | TCTTTCGCTCTGGTTTTTAATGAGAT | SEQ ID NO: 462 | TCTTTCGCTCTGGTTTTAAATGAGAC | SEQ ID NO: 2828 | TGGTAGACATGATTTGGACATC | SEQ ID NO: 5194 |
| rs3135353 | CATCACTAAGCTAAAGGGACAAGCTCTA | SEQ ID NO: 463 | CATCACTTAGCTAAAGGGACAAGCTCTG | SEQ ID NO: 2829 | GAAATGTGTCATAAGGTGTTTTC | SEQ ID NO: 5195 |
| rs3130235 | GTTCCTTTCTCCTTAGTGCCATGAA | SEQ ID NO: 464 | GTTGCTTTCTCCTTAGTGCCATGAG | SEQ ID NO: 2830 | AATCAAAACCCAACATGATTG | SEQ ID NO: 5196 |
| rs2394606 | TTCTACTGGCATGAAGACAGACAGAG | SEQ ID NO: 465 | TTCTACTGGCATGAAGACAGACAGAC | SEQ ID NO: 2831 | GAAATCTCAAGGGTGTTGTCT | SEQ ID NO: 5197 |
| rs2734997 | TGGTCATAAAATGAATAAAGCACTGGT | SEQ ID NO: 466 | TGGTCATAAAATGAATAAAGCACTGGC | SEQ ID NO: 2832 | TATGCTAAAACATTGCTGAATCT | SEQ ID NO: 5198 |
| rs3998794 | TTGGTCTCTTCTGAAGATTTCACAAAA | SEQ ID NO: 467 | TTGGTCTCTTCTGAAGATTTCACAAAG | SEQ ID NO: 2833 | TGGGTTCATGAAATATGTATCC | SEQ ID NO: 5199 |
| rs2844463 | TGAGAAGAACTGCTGCTCTCCCTAT | SEQ ID NO: 468 | TGAGAAGAACTGCTGCTCTCCCTAC | SEQ ID NO: 2834 | GCAAGGAAATCCAAATGGTAT | SEQ ID NO: 5200 |
| rs7452233 | AAAGAAGGGCAATGGAAGAAGGAT | SEQ ID NO: 469 | AAAGAAGGGCAATGGAAGAAGGAC | SEQ ID NO: 2835 | GTCTCCAACTCTGAAGGGAG | SEQ ID NO: 5201 |
| rs6910598 | AAACCAGAAGGCCCCCAGGTC | SEQ ID NO: 470 | AAACCAGAAGGCCCCCAGGTG | SEQ ID NO: 2836 | ATCTCTGGTTTCCACACACAC | SEQ ID NO: 5202 |
| rs3129008 | ACATCATGGGCCTCAATTGCTA | SEQ ID NO: 471 | ACATCATGGGCCTCAATTGCTG | SEQ ID NO: 2837 | TGAGTTGGACATTCAGAGGT | SEQ ID NO: 5203 |
| rs1150741 | GGAGGTTTCCGGTGTCAGCTA | SEQ ID NO: 472 | GGAGGTTTCCGGTGTCAGCTG | SEQ ID NO: 2838 | CTCTGGTTGTCTCCATAACCA | SEQ ID NO: 5204 |
| rs7774452 | TCTGCTTAGTTAGGAAGTTCCGAAT | SEQ ID NO: 473 | TCTGCTTAGTTAAGGAAGTTCCGAAC | SEQ ID NO: 2839 | CAGATAACTCAGAATCAGGTTTAA | SEQ ID NO: 5205 |
| rs6911724 | GGAAAGGCTGGAGAATTTAAGAACAAAT | SEQ ID NO: 474 | GGAAAGGCTGGAGAATTTAAGAACAAAC | SEQ ID NO: 2840 | ATTTAGACAAGTACTACAACCCCC | SEQ ID NO: 5206 |
| rs2294479 | CGTACCCAGAGTGGAGTGAACAATCTC | SEQ ID NO: 475 | CGTACCCAGAGTGGAGTGAACAATCTG | SEQ ID NO: 2841 | AGACTAACTCTTGCAGGATGC | SEQ ID NO: 5207 |
| rs417374 | TGATTTCTGGAATGGATGCCTGA | SEQ ID NO: 476 | TGATTTCTGGAATGGATGCCTGG | SEQ ID NO: 2842 | ATCAGAGCCAGGTATTTCAGG | SEQ ID NO: 5208 |
| rs1633030 | AGTCCAGTCCAGGGCTGCATCA | SEQ ID NO: 477 | AGTCCAGTCCAGGGCTGCATCG | SEQ ID NO: 2843 | CTGAGAGCTTCACTAGGGCTT | SEQ ID NO: 5209 |
| rs2024679 | GCGTCAACATTCCCAATGTGTGT | SEQ ID NO: 478 | GCGTCAACATTCCCAATGTGTGC | SEQ ID NO: 2844 | CCATTTTTCCTTTCTCACAC | SEQ ID NO: 5210 |
| rs3116999 | ACTGTTTTTTACCTCCACAGGGTTC | SEQ ID NO: 479 | ACTGTTTTTTACCTCCACAGGGTTG | SEQ ID NO: 2845 | TGACCTTGCCTAAAAGACTAAT | SEQ ID NO: 5211 |
| rs3765605 | CCAAGACACACGTGACCCACCA | SEQ ID NO: 480 | CCAAGACACACGTGACCCACCC | SEQ ID NO: 2846 | CCTCTCTGAACATGAGGCATA | SEQ ID NO: 5212 |
| rs2844603 | ACAAGGGGTGTCTGCTGCTGTA | SEQ ID NO: 481 | ACAAGGGGTGTCTGCTGCTGTG | SEQ ID NO: 2847 | CCACACTTGGAGAAAGAAAC | SEQ ID NO: 5213 |
| rs2621419 | TCACTTTTCCAGACAAAATACATGGAT | SEQ ID NO: 482 | TCACTTTTCCAGACAAAATACATGGAC | SEQ ID NO: 2848 | TGGTGATGCTTTAATCTAAATAGT | SEQ ID NO: 5214 |
| rs1362073 | TTTGGCTTCATAGCTGGGAAGAAA | SEQ ID NO: 483 | TTTGGCTTCATAGCTGGGAAGAAG | SEQ ID NO: 2849 | TTTGAAAAACAATCTAACTTTTACT | SEQ ID NO: 5215 |
| rs2072634 | CATCTTCCAGGTTTTCCATATCCTTA | SEQ ID NO: 484 | CATCTTCCAGGTTTTCCATATCCTTG | SEQ ID NO: 2850 | CTTTGAACACATGTTGCTCAT | SEQ ID NO: 5216 |
| rs213208 | CAGCATCCTAGGAGCTGACCACAT | SEQ ID NO: 485 | CAGCATCCTAGGAGCTGACCACAG | SEQ ID NO: 2851 | CTGATCATTAGGGCTGGAAAT | SEQ ID NO: 5217 |
| rs3956934 | TGTTCTACACCCAGAGTCACAAACTG | SEQ ID NO: 486 | TGTTCTACACCCAGAGTCACAAACTC | SEQ ID NO: 2852 | TATGTCAGCATTACCAGTTAGAAG | SEQ ID NO: 5218 |
| rs3117227 | GGTGGTCCACCCAACAGACAACA | SEQ ID NO: 487 | GGTGGTCCACCCAACAGACAACG | SEQ ID NO: 2853 | TGCCTCAGATGGTTATCAAG | SEQ ID NO: 5219 |
| rs4713215 | CAAGTCAGCGTTAATAGGGGGCAA | SEQ ID NO: 488 | CAAGTCAGCGTTAATAGGGGGCAG | SEQ ID NO: 2854 | TTTTAAGTGCACCACCACTT | SEQ ID NO: 5220 |
| rs1632942 | CCTTGTGCTAGGCCAGGCTGA | SEQ ID NO: 489 | CCTTGTGCTAGGCCAGGCTGG | SEQ ID NO: 2855 | AGGTCTACAGGAGATCAGGGA | SEQ ID NO: 5221 |
| rs3129886 | TGTAACTGATTTTCCCTCCAAGGGT | SEQ ID NO: 490 | TGTAACTGATTTTCCGTCCAAGGGC | SEQ ID NO: 2856 | TAACCTTGCCATTAACAAGC | SEQ ID NO: 5222 |
| rs2071798 | CAAATGTTACCTACTAAGGGATGCCTGA | SEQ ID NO: 491 | CAAATGTTACCTACTAAGGGATGCCTGG | SEQ ID NO: 2857 | TAAGCCACTCAGCTACCTAATTC | SEQ ID NO: 5223 |
| rs2523586 | GCTGAGCAGCTGCTGAGTATTGCT | SEQ ID NO: 492 | GCTGAGCAGCTGCTGAGTATTGCG | SEQ ID NO: 2858 | GAATCCCAGCTTATTCATTG | SEQ ID NO: 5224 |
| rs3117234 | CAGGGTTGGAGATCTGTTGATGAA | SEQ ID NO: 493 | CAGGGTTGGAGATCTGTTGATGAG | SEQ ID NO: 2859 | GTTGAAACCAATGACGATGAG | SEQ ID NO: 5225 |
| rs2071473 | TCAGGGACTTCGAGGGCTATTGTA | SEQ ID NO: 494 | TCAGGGACTTCGAGGGCTATTGTG | SEQ ID NO: 2860 | CCTCAAAGAGAAGCAGTAATT | SEQ ID NO: 5226 |
| rs3094041 | CATTAGCAATTTCACAATCTTGGGGT | SEQ ID NO: 495 | CATTAGCAATTTCACAATCTTGGGGC | SEQ ID NO: 2861 | ATTTTTGCTAAATATTTATCTTCCT | SEQ ID NO: 5227 |
| rs2596472 | CCAGATGTCTGAAAGGTTGAAGCCA | SEQ ID NO: 496 | CCAGATGTCTGAAAGGTTGAAGCCG | SEQ ID NO: 2862 | ATGTTGCTGAGACCATTGCTAT | SEQ ID NO: 5228 |
| rs2213570 | ATACAGCTGGCCCTGTATTTGTGT | SEQ ID NO: 497 | ATACAGCTGGCCCTGTATTTGTGG | SEQ ID NO: 2863 | TTTTGCATCCTCTGATTTAGT | SEQ ID NO: 5229 |
| rs1736922 | GAGAGACACCCCAGAGCCCTGA | SEQ ID NO: 498 | GAGAGACACCCCAGAGCCCTGG | SEQ ID NO: 2864 | CACTGTCAGTGATTGGGGTAG | SEQ ID NO: 5230 |
| rs3129269 | TATTTCAGTGAGCTGCCCCATCA | SEQ ID NO: 499 | TATTTCAGTGAGCTGCCCCATCC | SEQ ID NO: 2865 | CATGTTTGGTGTCTTCATCC | SEQ ID NO: 5231 |
| rs6920457 | TCTTCAATAAAGGAAACCAAGGAGAA | SEQ ID NO: 500 | TCTTCAATAAAGGAAACCAAGGAGAC | SEQ ID NO: 2866 | GTTGCCAGAAGTCAGATTAGAG | SEQ ID NO: 5232 |
| rs4711229 | TTTCCAACCACTGACAGTTTAACAAA | SEQ ID NO: 501 | TTTCCAACCACTGACAGTTTAACAAC | SEQ ID NO: 2867 | AGCTTGCAAATTTCCTGAATAT | SEQ ID NO: 5233 |
| rs1799964 | CCTCCAGACCCTGACTTTTCCTTCA | SEQ ID NO: 502 | CCTCCAGACCCTGACTTTTCCTTCG | SEQ ID NO: 2868 | CTTCTCAGCTTCTCCTTTGCT | SEQ ID NO: 5234 |
| rs4959037 | TCGAACCTCACCTTCTGTCTAGAGA | SEQ ID NO: 503 | TCGAACCTCACCTTCTGTCTAGAGT | SEQ ID NO: 2869 | ACAACAGGAGTGATTTATATTCC | SEQ ID NO: 5235 |
| rs1611652 | GCAGGGCCTTGGGAGATTACCG | SEQ ID NO: 504 | GCAGGGCCTTGGGAGATTACCC | SEQ ID NO: 2870 | GCATTACTCAAACCTTCTGC | SEQ ID NO: 5236 |
| rs2071354 | GGTGGAGCAGGAGCCCACAT | SEQ ID NO: 505 | GGTGGAGCAGGAGCCCACAC | SEQ ID NO: 2871 | CCTTGGACAAAAAGGAGAGAA | SEQ ID NO: 5237 |
| rs1737012 | TTAATTTCCTCTCTTGAGTGTGGCTT | SEQ ID NO: 506 | TTAATTTCCTCTCTTGAGTGTGGCTG | SEQ ID NO: 2872 | GACTTAATGATGCAAACTGATAT | SEQ ID NO: 5238 |
| rs1988564 | GCTTCTTTGCTTCTCAAACACCCAT | SEQ ID NO: 507 | GCTTCTTTGCTTCTCAAACACCCAC | SEQ ID NO: 2873 | ACCACATCTCAGATTGCATC | SEQ ID NO: 5239 |
| rs2071790 | CCACATCCACTTGGATTCCAGGA | SEQ ID NO: 508 | CCACATCCACTTGGATTCCAGGG | SEQ ID NO: 2874 | GTAAGCCTCTCGACTTCAGG | SEQ ID NO: 5240 |
| rs465151 | GTCAGTGGCCTCACTGTCAGCA | SEQ ID NO: 509 | GTCAGTGGCCTCACTGTCAGCG | SEQ ID NO: 2875 | TCAGTTGGGGTGAGATAGTC | SEQ ID NO: 5241 |
| rs1265759 | GCTATCACAAATAATGCTGCAAAGACAA | SEQ ID NO: 510 | GCTATCACAAATAATGCTGCAAAGACAG | SEQ ID NO: 2876 | CCTTAAGTAGATGGAAAAGAGTTT | SEQ ID NO: 5242 |
| rs2239701 | TGAGAGACAACTGAGAGACATTCCAT | SEQ ID NO: 511 | TGAGAGACAACTGAGAGACATTCCAC | SEQ ID NO: 2877 | TGAGACTTAAATTCCTTTTGTACT | SEQ ID NO: 5243 |
| rs3129883 | TCTTGGCCCAATCTCTCTCCACT | SEQ ID NO: 512 | TCTTGGCCCAATCTCTGTCCACC | SEQ ID NO: 2878 | CTTCCTGCCTACATGTATGTAG | SEQ ID NO: 5244 |
| rs2032502 | ACTCCCCCCTTATGGAGTCCCA | SEQ ID NO: 513 | ACTCCCCCCTTATGGAGTCCCG | SEQ ID NO: 2879 | AGTAGAGTAGGTGGACATCACTA | SEQ ID NO: 5245 |
| rs3778612 | GCATATGAGGGCACACCTGGTTA | SEQ ID NO: 514 | GCATATGAGGGCACACCTGGTTC | SEQ ID NO: 2880 | TTGTAGGGATTAAATTAATTTTTCT | SEQ ID NO: 5246 |
| rs2248871 | CTTGAATGAAGGACAACATGCATGT | SEQ ID NO: 515 | CTTGAATGAAGGACAACATGCATGG | SEQ ID NO: 2881 | GAAAGGTGGACAAATCACAG | SEQ ID NO: 5247 |
| rs2239707 | CATCTTGAATCCTGCTGGTCACTCA | SEQ ID NO: 516 | CATCTTGAATCCTGCTGGTCACTCG | SEQ ID NO: 2882 | TCCCTTGGTCTTAAAGCAAT | SEQ ID NO: 5248 |
| rs2734916 | CTCCCTTCCAGAGGTGCAGCTT | SEQ ID NO: 517 | CTCCCTTCCAGAGGTGCAGCTG | SEQ ID NO: 2883 | ATTCCCTCTCTTGAGTGTGG | SEQ ID NO: 5249 |
| rs2301753 | ATAGTGGCTCTCCTCGTCGTCCT | SEQ ID NO: 518 | ATAGTGGCTCTCCTCGTCGTCCG | SEQ ID NO: 2884 | ATCCTCCCCAGAAGAGTCTCT | SEQ ID NO: 5250 |
| rs2735042 | TGAGACTGTACGAATTTTATGACTGCAT | SEQ ID NO: 519 | TGAGACTGTACGAATTTTATGACTGCAC | SEQ ID NO: 2885 | ACAAATTATCACAAATATTGGCTT | SEQ ID NO: 5251 |
| rs3179375 | GACAGTGCCCAGGGCTCTGATA | SEQ ID NO: 520 | GACAGTGCCCAGGGCTCTGATG | SEQ ID NO: 2886 | GTCCCTCACAGCTTGTAAAG | SEQ ID NO: 5252 |
| rs3129869 | TGGCCCATTCTCCTGACTCTTTAA | SEQ ID NO: 521 | TGGCCCATTCTCCTGACTCTTTAC | SEQ ID NO: 2887 | TTTCAGGAAAGGTATTCCTG | SEQ ID NO: 5253 |
| rs7143 | GCTATATCTGCTCAGAGCTCACAAATGT | SEQ ID NO: 522 | GCTATATCTGCTCAGAGCTCACAAATGC | SEQ ID NO: 2888 | TTTGAATTATTTCCCTGACTTC | SEQ ID NO: 5254 |
| rs2068204 | TTCCAGAGCCAGCTAAGAATAGTACAA | SEQ ID NO: 523 | TTCCAGAGCCAGCTAAGAATAGTACAG | SEQ ID NO: 2889 | AGAACAGGATTGTGGACTAATTT | SEQ ID NO: 5255 |
| rs2745411 | CACAATGACACCCACCCCTCA | SEQ ID NO: 524 | CAGAATGACACCCACCCCTCG | SEQ ID NO: 2890 | CTTCTTCCTGAAATACCTGGC | SEQ ID NO: 5256 |
| rs462093 | GACCCCCATTCCCTGCCTTA | SEQ ID NO: 525 | GACCCCCATTCCCTGCCTTT | SEQ ID NO: 2891 | GGGACTGTCTTTTCTCGTTTT | SEQ ID NO: 5257 |
| rs3817966 | TCAGAGAACTCAGAGGAGTGACTCTTCT | SEQ ID NO: 526 | TCAGAGAACTCAGAGGAGTGACTCTTCC | SEQ ID NO: 2892 | CTTAAACCTTCTTGAGAGACAGAC | SEQ ID NO: 5258 |
| rs2256919 | GTCGTTTTCCTGGCACTGTTTCTT | SEQ ID NO: 527 | GTCGTTTTCCTGGCACTGTTTCTG | SEQ ID NO: 2893 | AAAAGATGCACTGATCCTCC | SEQ ID NO: 5259 |
| rs3957010 | TCCTTTTACATTATTTGTCTCCCCAGTT | SEQ ID NO: 528 | TCCTTTTACATTATTTGTCTCCCCAGTC | SEQ ID NO: 2894 | TACCTTCTGTGTTTCTCTTTGAT | SEQ ID NO: 5260 |
| rs1610710 | AGATGGGGTAAGGAGACGAATGAGA | SEQ ID NO: 529 | AGATGGGGTAAGGAGACGAATGAGG | SEQ ID NO: 2895 | GTCATGTCTCTTCTCAGGCAA | SEQ ID NO: 5261 |
| rs2735067 | GGCTCACTCCTCATGCCATTCA | SEQ ID NO: 530 | GGCTCACTCCTCATGCCATTCG | SEQ ID NO: 2898 | TTCTCTGCTCAAATGTCCCTTA | SEQ ID NO: 5262 |
| rs3126963 | GGAAAATGCTGCTGACTTGTTTTTGT | SEQ ID NO: 531 | GGAAAATGCTGCTGACTTGTTTTTGC | SEQ ID NO: 2897 | TAGTAATTTCAGCTGTCACATAATAA | SEQ ID NO: 5263 |
| rs7382938 | GTCCCTTCCCACAAGGCCATA | SEQ ID NO: 532 | GTCCCTTCCCACAAGGCCATG | SEQ ID NO: 2898 | CTCAGGCTGTCTCTCTCAGTG | SEQ ID NO: 5264 |
| rs3828901 | TAAGATTCAAGACAGGGCACAGTCA | SEQ ID NO: 533 | TAAGATTCAAGACAGGGCACAGTCG | SEQ ID NO: 2899 | TACCATCAGGAAGGTTCAAAC | SEQ ID NO: 5265 |
| rs3131043 | CCTGCAGTTGCCTTCAATGACTCT | SEQ ID NO: 534 | CCTGCAGTTGCCTTCAATGACTCC | SEQ ID NO: 2900 | ATTCTGGTTACCTGCTGCTC | SEQ ID NO: 5266 |
| rs2107197 | CATTCTCCCTTGAACCTCAACTCTATCT | SEQ ID NO: 535 | CATTCTCCCTTGAACCTCAACTCTATCC | SEQ ID NO: 2901 | TTCCCAGAGACTTGAAATAAAA | SEQ ID NO: 5267 |
| rs3106190 | GTGGGAGGGGTTCAGAAGCAGG | SEQ ID NO: 536 | GTGGGAGGGGTTCAGAAGCAGC | SEQ ID NO: 2902 | GCGTTTTTTCTCTGCTCTTC | SEQ ID NO: 5268 |
| rs1633102 | TCAGAAAAGGTAACTGGGCATGACA | SEQ ID NO: 537 | TGAGAAAAGGTAACTGGGCATGACG | SEQ ID NO: 2903 | TTCTGAGAACTTGACCATGG | SEQ ID NO: 5269 |
| rs3117120 | GCTCAATGATGGGGAAGGAAAAGG | SEQ ID NO: 538 | GCTCAATGATGGGGAAGGAAAAGC | SEQ ID NO: 2904 | GGTGAGGGACAAAATCTCAG | SEQ ID NO: 5270 |
| rs4713610 | TGGGTGAGTTACTTTACTTCTGTGGTCA | SEQ ID NO: 539 | TGGGTGAGTTACTTTACTTCTGTGGTCC | SEQ ID NO: 2905 | TTCCCCAATATGTAAAATAATTTC | SEQ ID NO: 5271 |
| rs2076522 | ACACTTTATTTGTTCCCCAGTCTGG | SEQ ID NO: 540 | ACACTTTATTTGTTCCCCAGTCTGC | SEQ ID NO: 2906 | TCTTTACATTTTAGCATCTGACC | SEQ ID NO: 5272 |
| rs2853937 | GCTCCCCAAGGCATAGTTTAATATGT | SEQ ID NO: 541 | GCTCCCCAAGGCATAGTTTAATATGC | SEQ ID NO: 2907 | TTTCTATTATCTGTATTGCCTCTAA | SEQ ID NO: 5273 |
| rs867464 | TCTCTTCATCCCTGTTCTTCACATT | SEQ ID NO: 542 | TCTCTTCATCCCTGTTCTTCACATC | SEQ ID NO: 2908 | GTATATCCAACTACCTACCTGTTG | SEQ ID NO: 5274 |
| rs2074466 | GCGGCCCCAATACCATCCA | SEQ ID NO: 543 | GCGGCCCCAATACCATCCC | SEQ ID NO: 2909 | CAGTTCTTGTGTGAGATCCAG | SEQ ID NO: 5275 |
| rs7382649 | GATGACTGGCGTGCTCCTAACAA | SEQ ID NO: 544 | GATGACTGGCGTGCTCCTAACAG | SEQ ID NO: 2910 | TGTTCAGACAGAAGGCACCTA | SEQ ID NO: 5276 |
| rs7452209 | CCTAGTAAGAGTCATGCTGAGGGAAGAT | SEQ ID NO: 545 | CCTAGTAAGAGTCATGCTGAGGGAAGAG | SEQ ID NO: 2911 | TTTGAATAATCATTTCATCATTACA | SEQ ID NO: 5277 |
| rs5007238 | CTCGAGGGCGTTTTTCCTTGA | SEQ ID NO: 546 | CTCGAGGGCGTTTTTCCTTGC | SEQ ID NO: 2912 | GGTATTTTCAGTTCTATTAACACTTC | SEQ ID NO: 5278 |
| rs757262 | CCTGGTCATTGATCTGGAAAGGAT | SEQ ID NO: 547 | CCTGGTCATTGATCTGGAAAGGAC | SEQ ID NO: 2913 | GCCAAGGAATTAGACACCAAC | SEQ ID NO: 5279 |
| rs1611407 | TTTAGGCCAGATTTGGTGGCTCA | SEQ ID NO: 548 | TTTAGGCCAGATTTGGTGGCTCG | SEQ ID NO: 2914 | ACCTGCAATCCTAGCATTGT | SEQ ID NO: 5280 |
| rs1655937 | GAGATCTACAGGCGATCAGGGAGA | SEQ ID NO: 549 | GAGATCTACAGGCGATCAGGGAGG | SEQ ID NO: 2915 | GTCTTGTGTTCCCTGGTACC | SEQ ID NO: 5281 |
| rs2269704 | GGCCACGAGGGAGAAGCAA | SEQ ID NO: 550 | GGCCACGAGGGAGAAGCAG | SEQ ID NO: 2916 | GAGACAGTAGAAGAGCATGGG | SEQ ID NO: 5282 |
| rs2734331 | CCTCCTGCCTCATCATGACCACA | SEQ ID NO: 551 | CCTCCTGCCTCATCATGACCACG | SEQ ID NO: 2917 | AGATCCTTCGGTGAGAGATG | SEQ ID NO: 5283 |
| rs3998178 | TGTACAGGAGCTTTGGAATACTGGT | SEQ ID NO: 552 | TGTACAGGAGCTTTGGAATACTGGC | SEQ ID NO: 2918 | AGCATCACTGTCTCCAPAAGTTA | SEQ ID NO: 5284 |
| rs885914 | GGAAGGTGGCAATACAGAAAAAAAA | SEQ ID NO: 553 | GGAAGGTGGCAATACAGAAAAAAAC | SEQ ID NO: 2919 | AATCAGGTGTTTTTTTTTAAGC | SEQ ID NO: 5285 |
| rs6925041 | TGCTAGTATCTGTATTTGGTGCTGTGT | SEQ ID NO: 554 | TGCTAGTATCTGTATTTGGTGCTGTGC | SEQ ID NO: 2920 | CCTTCTACCCTTCAATGTAAAGA | SEQ ID NO: 5266 |
| rs441126 | TCATATCTTTTGGCACCTTCTAGAATGT | SEQ ID NO: 555 | TCATATCTTTTGGCACCTTCTAGAATGC | SEQ ID NO: 2921 | TTCTCTGATTAGAATTCTTCTTCC | SEQ ID NO: 5287 |
| rs2394604 | GGCCTGTCCTAAGGAGATGTATGAGTT | SEQ ID NO: 556 | GGCCTGTCCTAAGGAGATGTATGAGTG | SEQ ID NO: 2922 | GGTACTTATCTAATTGACTTAACCC | SEQ ID NO: 5288 |
| rs2074502 | AACAGCAGCAGCCGCAACATAT | SEQ ID NO: 557 | AACAGCAGCAGCCGCAACATAC | SEQ ID NO: 2923 | GAACGGTTGCCATAGACTCT | SEQ ID NO: 5289 |
| rs3815100 | CACCTGCAACAGACTCAGCACAGA | SEQ ID NO: 558 | CACCTGCAACAGACTCAGCACAGC | SEQ ID NO: 2924 | AACATGCAAATTCTGGAAGGT | SEQ ID NO: 5290 |
| rs1632932 | GTTTTTCCTGATCCCGCCCTGA | SEQ ID NO: 559 | GTTTTTCCTGATCCCGCCCTGG | SEQ ID NO: 2925 | TCTGCAGTCACACATTTCTG | SEQ ID NO: 5291 |
| rs2076524 | GCTCTGCCTCTAGGACCCACAT | SEQ ID NO: 560 | GCTCTGCCTCTAGGACCCACAC | SEQ ID NO: 2926 | CTCAGATCAGCAGGAGAGGT | SEQ ID NO: 5292 |
| rs7759127 | GGGTTTCTTTGCATTCGGTATTTAATACA | SEQ ID NO: 561 | GGGTTCTGCATTCGGTATTTAATACC | SEQ ID NO: 2927 | TTTGTGTGACTGCCTTAAAACTA | SEQ ID NO: 5293 |
| rs3765606 | GGGGGGTCTGAAAGGAAGAGTCA | SEQ ID NO: 562 | GGGGGGTCTGAAAGGAAGAGTCG | SEQ ID NO: 2928 | AAAACTCAGGCACTTTGCAT | SEQ ID NO: 5294 |
| rs7381988 | CCTGTCAGTGGTGACAAATAACACTTAA | SEQ ID NO: 563 | CCTGTCAGTGGTGACAAATAACACTTAG | SEQ ID NO: 2929 | AAGTATTGCAGTAAGCATCACTT | SEQ ID NO: 5295 |
| rs2071479 | AGACTCAGGGGACAGTCCCATCT | SEQ ID NO: 564 | AGACTCAGGGGACAGTCCCATCC | SEQ ID NO: 2930 | GACAGCAGCAACCTTACTCCT | SEQ ID NO: 5296 |
| rs4986978 | CCGACCCCCGAGAGAGAGATT | SEQ ID NO: 565 | CCGACCCCCGAGAGAGAGATC | SEQ ID NO: 2931 | ACAGAGAAGGGGACAAGATG | SEQ ID NO: 5297 |
| rs2071472 | TTCTTGCATACACACTGGAAAAAAAT | SEQ ID NO: 566 | TTCTTGCATACACACTGGAAAAAAAC | SEQ ID NO: 2932 | ATTGCTCTGTTCTTACCTGGAG | SEQ ID NO: 5298 |
| rs7349863 | CCTGTTTGTTGTCTAACGAACAATCAGT | SEQ ID NO: 567 | CCTGTTTGTTGTCTAACGAACAATCAGC | SEQ ID NO: 2933 | ACACACTCAATTCTAAGTAAAAACC | SEQ ID NO: 5299 |
| rs3093665 | CCCCCTCCTTCAGACACCCTCA | SEQ ID NO: 568 | CCCCCTCCTTCAGACACCCTCC | SEQ ID NO: 2934 | CCTCTTCTGGCTCAAAAAGAG | SEQ ID NO: 5300 |
| rs3132132 | TTCCCTTCTGCATTCAGGGTATGA | SEQ ID NO: 569 | TTCCCTTCTGCATTCAGGGTATGG | SEQ ID NO: 2935 | AGAAATAACTGTCTAGAACTCAATCT | SEQ ID NO: 5301 |
| rs3823318 | AATGGTCATGATGATGCACACAATC | SEQ ID NO: 570 | AATGGTCATGATGATGCACACAATG | SEQ ID NO: 2936 | GGGCTCAGATGTTCAGGTACT | SEQ ID NO: 5302 |
| rs2106072 | TTGACCACGACCTTGGCCCT | SEQ ID NO: 571 | TTGACCACGACCTTGGCCCC | SEQ ID NO: 2937 | GCTCTGCACTCTCTGGAATAA | SEQ ID NO: 5303 |
| rs3130654 | CACACACCTCAATTCCCATTGAGC | SEQ ID NO: 572 | CACACACCTCAATTCCCATTGAGG | SEQ ID NO: 2938 | TGGCTGGTCTGTAAATAGCATA | SEQ ID NO: 5304 |
| rs2857130 | CATATGAATTTTGGGACGGCATATA | SEQ ID NO: 573 | CATATGAATTTTGGGACGGCATATT | SEQ ID NO: 2939 | AGTCCACACCAGGAAGTTCTT | SEQ ID NO: 5305 |
| rs1264300 | CGGTCTCCTCCAGCCCCTCT | SEQ ID NO: 574 | CGGTGTCCTCCAGCCCCTCC | SEQ ID NO: 2940 | AAGTCTCAGAAACCCAAGGAC | SEQ ID NO: 5308 |
| rs909727 | ACTGTGGAGGGAAGGGAGACCTG | SEQ ID NO: 575 | ACTGTGGAGGGAAGGGAGACCTC | SEQ ID NO: 2941 | GTCAGGAGAGCTGAGACCAC | SEQ ID NO: 5307 |
| rs4151670 | GCCACCTCTTGAGGGGTGTCA | SEQ ID NO: 576 | GCCACCTCTTGAGGGGTGTCG | SEQ ID NO: 2942 | ACATGAAGGAGTCTGGGAGAG | SEQ ID NO: 5308 |
| rs1419637 | GCTGGTATAGGCAAATGTTTACAGAAT | SEQ ID NO: 577 | GCTGGTATAGGCAAATGTTTACAGAAA | SEQ ID NO: 2943 | TAATTGTAAAGGGGAACACAGA | SEQ ID NO: 5309 |
| rs2532934 | CTGTCCTGGGCTGGGTCCTCTA | SEQ ID NO: 578 | CTGTCCTGGGCTGGGTCCTCTG | SEQ ID NO: 2944 | GGTGCTAGATGTACAATGCC | SEQ ID NO: 5310 |
| rs1632981 | GCTTTGCAGACACCATTCAATCTTAA | SEQ ID NO: 579 | GCTTTGCAGACACCATTCAATCTTAT | SEQ ID NO: 2945 | AGGTTCCAAAAACAAGAATGG | SEQ ID NO: 5311 |
| rs7383287 | CAGCCTTTGCCTGAATCACAAAA | SEQ ID NO: 550 | CAGCCTTTGCCTGAATCACAAAG | SEQ ID NO: 2946 | CTTCTGGAAAACCAAAACCA | SEQ ID NO: 5312 |
| rs2071477 | TCTCAAAATAACCGTCTCATGAGGTA | SEQ ID NO: 581 | TCTCAAAATAACCGTCTCATGAGGTG | SEQ ID NO: 2947 | TATATTATCTCCACTTTACAGATGC | SEQ ID NO: 5313 |
| rs2071652 | TCCTGAGGTTGTTTTCAGGCTGT | SEQ ID NO: 582 | TCCTGAGGTTGTTTCCAGGCTGC | SEQ ID NO: 2948 | GAGAAATAGCCTGCACAAGG | SEQ ID NO: 5314 |
| rs7756516 | AAGCATGACAAACAAAATCATTTACCTA | SEQ ID NO: 583 | AAGCATGACAAACAAAATCATTTACCTG | SEQ ID NO: 2949 | CTTTAGTGCTTTTTCCCATAATT | SEQ ID NO: 5315 |
| rs2734981 | GCAGAAAGACTCGGGTTTCCCA | SEQ ID NO: 584 | GCAGAAAGAGTCGGGTTTCCCG | SEQ ID NO: 2950 | ACACTCCAGGCTCTCAGTGT | SEQ ID NO: 5316 |
| rs3823339 | GAACCTGGTCAGATCCCACAGAAC | SEQ ID NO: 585 | GAACCTGGTCAGATCCCACAGAAG | SEQ ID NO: 2951 | TGTGGCTAGAGGAGGAATTGT | SEQ ID NO: 5317 |
| rs7770557 | CCCCTGGAAAGGGTTCCAAGTC | SEQ ID NO: 586 | CCCCTGGAAAGGGTTCCAAGTG | SEQ ID NO: 2952 | TTTAGTACCCGACGCTGTCT | SEQ ID NO: 5318 |
| rs1054175 | TGTATAAAAGTGCCCAAAAGCGTTAC | SEQ ID NO: 587 | TGTATAAATGTGCCCAAAAGCGTTAG | SEQ ID NO: 2953 | ACAGGACTCACAAAGCTTCTAA | SEQ ID NO: 5319 |
| rs3131786 | TCTCACCTAGATGGCTGCAGCTG | SEQ ID NO: 588 | TCTCACCTAGATGGCTGCAGCTC | SEQ ID NO: 2954 | CAGAGCTTTCCATTCCTTTG | SEQ ID NO: 5320 |
| rs2844686 | CTCAACCACCCTTGGGGATGT | SEQ ID NO: 589 | CTCAACCACCCTTGGGGATGC | SEQ ID NO: 2955 | TACTCCTATTATCCCATTTTATAGAC | SEQ ID NO: 5321 |
| rs1611653 | GAGACAAGGTTTACTGGATGTGCCA | SEQ ID NO: 590 | GAGACAAGGAACTGGATGTGCCG | SEQ ID NO: 2956 | AGACCACAGGCAATCTAAGG | SEQ ID NO: 5322 |
| rs2242655 | GTGCTAGAGATAGGCTGGTCCCAG | SEQ ID NO: 591 | GTGCTAGAGATAGGCTGGTCCCAC | SEQ ID NO: 2957 | TGAGGGAATCCATTCTTTTG | SEQ ID NO: 5323 |
| rs969891 | ACTCCAAGTCCTGAGGTTGTCCAA | SEQ ID NO: 592 | ACTCCAAGTCCTGAGGTTGTCCAG | SEQ ID NO: 2958 | TACCCCCAGGACATCATTAAAT | SEQ ID NO: 5324 |
| rs2023609 | CAGGTGGAGGGGAGGGATGA | SEQ ID NO: 593 | CAGGTGGAGGGGAGGGATGC | SEQ ID NO: 2959 | GCAGTGTGTTATTGCTCTCC | SEQ ID NO: 5325 |
| rs3129882 | TTCCATAGATTTGGCCCTTAAATAAAA | SEQ ID NO: 594 | TTCCATAGATTTGGCCCTTAAATAAAG | SEQ ID NO: 2960 | GAAGCTTCTGACTCTAAAATGTAAA | SEQ ID NO: 5326 |
| rs3132553 | GGAAGGGTGACTCCAGCGGT | SEQ ID NO: 595 | GGAAGGGTGACTCCAGCGGC | SEQ ID NO: 2961 | TCAGTAGCTACAGTGGCTCC | SEQ ID NO: 5327 |
| rs2233956 | CAGGTGGGTCCAGTGTAGTCACCA | SEQ ID NO: 596 | CAGGTGGGTCCAGTGTAGTCACCG | SEQ ID NO: 2962 | GTCCCTTAATGTGGACATGG | SEQ ID NO: 5328 |
| rs3128930 | GAATATACTGGCACATAAGCCAAGGAA | SEQ ID NO: 597 | GAATATACTGGCACATAAGCCAAGGAG | SEQ ID NO: 2963 | TAAAGATTTCCAGGAAGGAGG | SEQ ID NO: 5329 |
| rs3130560 | CAAATGATTAGCCCCATTTCACAGAA | SEQ ID NO: 598 | CAAATGATTAGCCCCATTTCACAGAC | SEQ ID NO: 2964 | AGGAACCAACACTGAGAGAG | SEQ ID NO: 5330 |
| rs1042141 | CAGGGGTTTCCCAGTTGAGAAGG | SEQ ID NO: 599 | CAGGGGTTTCCCAGTTGAGAAGC | SEQ ID NO: 2965 | GATGGGGGTGTTACTCAATG | SEQ ID NO: 5331 |
| rs2256543 | TTTTGTTGCAGCCACCCATAATA | SEQ ID NO: 600 | TTTTGTTGCAGCCACCCATAATG | SEQ ID NO: 2966 | ATGGCGACTGTCTCAAGACT | SEQ ID NO: 5332 |
| rs2071362 | ATCCCTGCACATGCCCTGGT | SEQ ID NO: 601 | ATCCCTGCACATGCCCTGGC | SEQ ID NO: 2967 | CTCCAGGTGAAGAGCACATAG | SEQ ID NO: 5333 |
| rs4711313 | GCCTCAAGGTGGCTAAGCTCCA | SEQ ID NO: 602 | GCCTCAAGGTGGCTAAGCTCCG | SEQ ID NO: 2968 | TTTGCATCTCTATTATTGAAAA | SEQ ID NO: 5334 |
| rs916568 | CGTTGTATAACAGGCTACCTCTGTGTT | SEQ ID NO: 603 | CGTTGTATAACAGGCTACCTCTGTGTC | SEQ ID NO: 2969 | AAAAAAGAGGGAATGAAGAGTC | SEQ ID NO: 5335 |
| rs4988889 | GCAGTTCCACCACTAATGTGCTTTAT | SEQ ID NO: 604 | GCAGTTCCACCACTAATGTGCTTTAG | SEQ ID NO: 2970 | AGAGTCAACAATTACTGAATTTATT | SEQ ID NO: 5336 |
| rs3132524 | TGAAACACTTCCTTGAGGCTTCAGA | SEQ ID NO: 605 | TGAAACACTTCCTTGAGGCTTCAGG | SEQ ID NO: 2971 | CTACAAAAAGGCCTTGTTTT | SEQ ID NO: 5337 |
| rs1033569 | GCAAACAAATTTCTAAGGACTGATTGTT | SEQ ID NO: 606 | GCAAACAAATTTCTAAGGACTGATTGTC | SEQ ID NO: 2972 | TTTTGTGTGTGTAATTTTGTTTG | SEQ ID NO: 5338 |
| rs4713242 | GGCTAGTCAAGTAAAGCAGTGGGAGT | SEQ ID NO: 607 | GGCTAGTCAAGTAAAGCAGTGGGAGC | SEQ ID NO: 2973 | GAGAAGGAAAAAAGAAATCTGTAA | SEQ ID NO: 5339 |
| rs3095334 | CAGAGGACACGCCATAGCTGGG | SEQ ID NO: 608 | CAGAGGACACGCCATAGCTGGC | SEQ ID NO: 2974 | GACATCACTGTCGAGGTGTG | SEQ ID NO: 5340 |
| rs2844666 | TTTGCTAACACACACAGAAAAGAAATGT | SEQ ID NO: 609 | TTTGCTAACACACACAGAAAAGAAATGG | SEQ ID NO: 2975 | TAATATGTGTTGGTGATGTGTTC | SEQ ID NO: 5341 |
| rs2517671 | TCTTCCCTAGTGTGCAAGTCCCTT | SEQ ID NO: 610 | TCTTCCCTAGTGTGCAAGTCCCTC | SEQ ID NO: 2976 | CCCAATTCCTCATTAGCTGAG | SEQ ID NO: 5342 |
| rs6903130 | CAGGAAATTCTTCTTCACAGCTTTTGT | SEQ ID NO: 611 | CAGGAAATTCTTCTTCACAGCTTTTGC | SEQ ID NO: 2977 | CTCTATCTTCAAAACTAAATGTGAC | SEQ ID NO: 5343 |
| rs2179919 | CCTCTCCCGGTGTCTGATTCCT | SEQ ID NO: 612 | CCTCTGCCGGTGTCTGATTCCC | SEQ ID NO: 2978 | CTCTGTAACATGAAATAAATAAGAACC | SEQ ID NO: 5344 |
| rs2233984 | TGGGGGAATATTCATCTATACCCAGA | SEQ ID NO: 613 | TGGGGGAATATTCATCTATACCCAGG | SEQ ID NO: 2979 | ATCAATAACCCATTTCCTCC | SEQ ID NO: 5345 |
| rs3117574 | GCCCGGCCCTGATAGCCA | SEQ ID NO: 614 | GCCCGGCCCTGATAGCCG | SEQ ID NO: 2980 | TGAGGTTTTTTTGTCATTGTTC | SEQ ID NO: 5346 |
| rs356969 | CAGCCTCCCCAGGGTCTCAC | SEQ ID NO: 615 | CAGCCTCCCCAGGGTCTCAG | SEQ ID NO: 2981 | TTTACAAGCCGTGAGAGACAC | SEQ ID NO: 5347 |
| rs3128972 | CCATGTCACTGAATCCAACAGTACATTAT | SEQ ID NO: 616 | CCATGTCACTGAATCCAACAGTACATTAC | SEQ ID NO: 2982 | AAAACAATACGTTTTGACCAATT | SEQ ID NO: 5348 |
| rs4084093 | CATTGCTTTGTCCCAGCAAGTTGT | SEQ ID NO: 617 | CATTGCTTTGTCCCAGCAAGTTGC | SEQ ID NO: 2983 | GACAAATCTTGAATATTCTAAAATAGG | SEQ ID NO: 5349 |
| rs805262 | GGGCTGTAGGGGGCGACCA | SEQ ID NO: 618 | GGGCTGTAGGGGGCGACCG | SEQ ID NO: 2984 | GTTTAGGGAAGCATGAGAAAC | SEQ ID NO: 5350 |
| rs2269555 | CCTGGTTTGGGTCAGGTATTGCA | SEQ ID NO: 619 | CCTGGTTTGGGTCAGGTATTGCG | SEQ ID NO: 2985 | AGATCTAATCTGCCCCAACC | SEQ ID NO: 5351 |
| rs3095298 | TCCTTCAAAAGCGGGCTGTA | SEQ ID NO: 620 | TTTCCTTCAAAAGCGGGCTGTG | SEQ ID NO: 2986 | TTCTCTTCTACCTTCCAGCT | SEQ ID NO: 5352 |
| rs189865 | AAAGGGAAATGTGAACTATCTTGCTT | SEQ ID NO: 621 | AAAGGGAAATGTGAACTATCTTGCTC | SEQ ID NO: 2987 | GTTGATGGGAGTGTAAACTGTT | SEQ ID NO: 5353 |
| rs3130991 | TTTGATCATTACAAGAACGGTATGAAAA | SEQ ID NO: 622 | TTTGATCATTACAAGAACGGTATGAAAG | SEQ ID NO: 2988 | AGATTTGCTATTTCCACTCTACA | SEQ ID NO: 5354 |
| rs1061235 | CCTTTGCAGAAACAAAGTCAGGGTA | SEQ ID NO: 623 | CCTTTGCAGAAACAAAGTCAGGGTT | SEQ ID NO: 2989 | TTCAAGTCACAAAGGGAAGG | SEQ ID NO: 5355 |
| rs3823421 | GGGAGAGGACGGCCATTCCA | SEQ ID NO: 624 | GGGAGAGGACGGCCATTCCG | SEQ ID NO: 2990 | ACATAGGGAACAGGACACAC | SEQ ID NO: 6356 |
| rs4079877 | TTTTTAACATTGTATTAGGCTGTGCCT | SEQ ID NO: 625 | TTTTTAACATTGTATTAGGCTGTGCCG | SEQ ID NO: 2991 | CACTGCTGTAAAGAAATACCTG | SEQ ID NO: 5357 |
| rs909968 | TGTTACCTAGCCCCTTTGCCACA | SEQ ID NO: 626 | TGTTACCTAGCCCCTTTGCCACG | SEQ ID NO: 2992 | AATATGGGGGCTTATCTCCAC | SEQ ID NO: 5358 |
| rs3218807 | GTGGGGAAGCCGAAAGGAGA | SEQ ID NO: 627 | GTGGGGAAGCCGAAAGGAGG | SEQ ID NO: 2993 | GAGCTGGCTTTTAATTGAGC | SEQ ID NO: 5359 |
| rs760293 | GCATGGAAGGGCAGGTCTGAAA | SEQ ID NO: 628 | GCATGGAAGGGCAGGTCTGAAG | SEQ ID NO: 2994 | TCTCCCTTGCTCTCTATCCAG | SEQ ID NO: 5360 |
| rs5026743 | CTCTGTCACCCATGCTGTAGTTCA | SEQ ID NO: 629 | CTCTGTCACCCATGCTGTAGTTCC | SEQ ID NO: 2995 | TGTTATGATCTAGGCTTACTGCA | SEQ ID NO: 5361 |
| rs2213586 | GGGGTTTCAGATTCACTCTACTGAAGAT | SEQ ID NO: 630 | GGGGTTTCAGATTCACTCTACTGAAGAC | SEQ ID NO: 2996 | GAGTTATATCATTGCTCAGTAGAGA | SEQ ID NO: 5362 |
| rs3132709 | GATTTCCAAGGACCCAGACCCA | SEQ ID NO: 631 | GAACCAAGGACCCAGACCCG | SEQ ID NO: 2997 | CAGGGCAGAAGGTTTGAGTA | SEQ ID NO: 5363 |
| rs805267 | CAGCTCCACTGCAATGGATTCCT | SEQ ID NO: 632 | CAGCTCCACTGCAATGGATTCCC | SEQ ID NO: 2998 | ACCATTGCTCAAGTCTCCAG | SEQ ID NO: 5364 |
| rs391755 | TGTTACAGACAATTCAGACTGCAAAAA | SEQ ID NO: 633 | TGTTACAGACAATTCAGACTGCAAAAT | SEQ ID NO: 2999 | TAGGGTAGACTATGTTCATTTAACA | SEQ ID NO: 5365 |
| rs3765604 | AGGGTAGAAGCCCAGAACCCAGG | SEQ ID NO: 634 | AGGGTAGAAGCCCAGAACCCAGC | SEQ ID NO: 3000 | CCTCGTTATGCCTCATGTTC | SEQ ID NO: 5366 |
| rs1265087 | CCCCAGAGTCAGTTTCTATCATATCATCA | SEQ ID NO: 635 | CCCCAGAGTCAGTTTCTATCATATCATCG | SEQ ID NO: 3001 | ACTGTACTTTCTCTTCAAGCACTT | SEQ ID NO: 5367 |
| rs516535 | AGAAGCAGCAGCACCAGGGAAA | SEQ ID NO: 636 | AGAAGCAGCAGCACCAGGGAAG | SEQ ID NO: 3002 | GGATTCGAAAACCCTCTCTCT | SEQ ID NO: 5368 |
| rs1632883 | GGTGAGGATGCAAGATGCAAGTT | SEQ ID NO: 637 | GGTGAGGATGCAAGATGCAAGTG | SEQ ID NO: 3003 | GTCTTTTACTTTGTGTGTGTGT | SEQ ID NO: 5369 |
| rs7740076 | AAAATATGTGGTCAAAGAAAAGGGGA | SEQ ID NO: 638 | AAAATATGTGGTCAAAGAAAAGGGGG | SEQ ID NO: 3004 | AATATGAGTTATCACAAAAACATTT | SEQ ID NO: 5370 |
| rs10885 | ACCACCTCCTGCCCCACCTT | SEQ ID NO: 639 | AGCACCTCCTGCCCCACCTC | SEQ ID NO: 3005 | CCTTTCTCTGTTACCTGTGG | SEQ ID NO: 5371 |
| rs1042149 | CCGCTTTTGAAGGAAAATGAGGA | SEQ ID NO: 640 | CCGCTTTTGAAGGAAAATGAGGG | SEQ ID NO: 3006 | CACAGAGACCTCTAGAGGCGTA | SEQ ID NO: 5372 |
| rs3129859 | AGAAGTGTGGAGAAGGAATCACTAGAGC | SEQ ID NO: 641 | AGAAGTGTGGAGAAGGAATCACTAGAGG | SEQ ID NO: 3007 | TCCTGATAAGAAAGATGGTAGAA | SEQ ID NO: 5373 |
| rs362546 | GGCTGGACTGTTCCCACACTCA | SEQ ID NO: 642 | GGCTGGACTGTTCCCACACTCG | SEQ ID NO: 3008 | CTGAGCAATCAATTCTGGCT | SEQ ID NO: 5374 |
| rs1633057 | GCCCCAGAACCCCTTCCACA | SEQ ID NO: 643 | GCCCCAGAACCCCTTCCACG | SEQ ID NO: 3009 | AATCTCAATATCCAGGCCTG | SEQ ID NO: 5375 |
| rs3807032 | GTAGCCCCCTGTGACCCCCG | SEQ ID NO: 644 | GTAGCCCCCTGTGACCCCCC | SEQ ID NO: 3010 | AAGAGCCCCAATTTTACCTT | SEQ ID NO: 5376 |
| rs3130212 | ACTAGGGTGAGGCCTCAGAAGACC | SEQ ID NO: 645 | ACTAGGGTGAGGCCTCAGAAGACG | SEQ ID NO: 3011 | GCTAAGCAGGTTGAAGTCGT | SEQ ID NO: 5377 |
| rs423639 | AAATGAGGGCAGTCCCACTCCA | SEQ ID NO: 646 | AAATGAGGGCAGTCCCACTCCG | SEQ ID NO: 3012 | GTTGGCCTCATGAAGACATC | SEQ ID NO: 5378 |
| rs3135000 | GGAATCTGTGCTTGGTCAGTGGAA | SEQ ID NO: 647 | GGAATCTGTGCTTGGTCAGTGGAG | SEQ ID NO: 3013 | TGAATGTGAACATAGGAGCC | SEQ ID NO: 5379 |
| rs1264620 | TGGCACTATCTGTACCAACATACAACT | SEQ ID NO: 648 | TGGCACTATCTGTACCATCATACAACC | SEQ ID NO: 3014 | TCTCACATTAAAGCTATTTTTCTTG | SEQ ID NO: 5380 |
| rs1264703 | CTGAAGGCAGGCCAACGTGG | SEQ ID NO: 649 | CTGAAGGCAGGCCAACGTGC | SEQ ID NO: 3015 | GTTCAGGCTTTATCCTTTAGAGA | SEQ ID NO: 5381 |
| rs1465651 | GGAAGGGGTTAACAGAAGAAGTCAGTGA | SEQ ID NO: 650 | GGAAGGGGTTAACAGAAGAAGTCAGTGG | SEQ ID NO: 3016 | AGGAACTTAATCTCTACATTTTACCA | SEQ ID NO: 5382 |
| rs7194 | GGATTGTAATATTGCCAGCTTTGTAAAGT | SEQ ID NO: 651 | GGATTGTAATATTGCCAGCTTTGTAAAGC | SEQ ID NO: 3017 | ATTAAAGCAGAAGTTTCTTCAGTG | SEQ ID NO: 5383 |
| rs213210 | CATCGGGACCTAGGTGTGTTCTCA | SEQ ID NO: 652 | CATCGGGACCTAGGTGTGTTCTCG | SEQ ID NO: 3018 | GACTAGAAGGCTAAAGCGGC | SEQ ID NO: 5384 |
| rs2256028 | GAGGGTTTCCCTGGACACATCTT | SEQ ID NO: 653 | GAGGGTTTCCCTGGACACATCTG | SEQ ID NO: 3019 | GAGGTGAGGCAACTCTAGCA | SEQ ID NO: 5385 |
| rs762815 | GGGACAGAGCGCGTGCGG | SEQ ID NO: 654 | GGGACAGAGCGCGTGCGC | SEQ ID NO: 3020 | GTGTGGCCAGATACATCTATAAC | SEQ ID NO: 5386 |
| rs3868542 | ATGAGGCCCTGCAGGGAAACA | SEQ ID NO: 655 | ATGAGGCCCTGCAGGGAAACG | SEQ ID NO: 3021 | GAGATTCCCTTCCCATTGTT | SEQ ID NO: 6387 |
| rs5021449 | CATTCAACAAGGACAGAGTGATTCTAAAT | SEQ ID NO: 656 | CATTCAACAAGGACAGAGTGATTCTAAAC | SEQ ID NO: 3022 | AAACCTCCATTACTAACTTGTTGT | SEQ ID NO: 5388 |
| rs1632880 | CAGGTGAGAAGAGGAGGAGGCAGC | SEQ ID NO: 657 | CAGGTGAGAAGAGGAGGAGGCAGG | SEQ ID NO: 3023 | CTGCTATCACACAATGAGGG | SEQ ID NO: 5389 |
| rs7774954 | CAGGGGCAGGTGGGCATTAA | SEQ ID NO: 658 | CAGGGGCAGGTGGGCATTAC | SEQ ID NO: 3024 | GAAGAGTGATGACCAATCCC | SEQ ID NO: 5390 |
| rs4713520 | AATACCCTACCTGGTTGTTGTGAGT | SEQ ID NO: 659 | AATACCCTACCTGGTTGTTGTGAGC | SEQ ID NO: 3025 | TCAAATGATTTAATACATAAAGTGC | SEQ ID NO: 5391 |
| rs6456895 | CCCTCGTTCTCTAATCAATTATCTTTGT | SEQ ID NO: 660 | CCCTCGTTCTCTAATCAATTATCTTTGC | SEQ ID NO: 3026 | TACTGTGGATTCATTGTAGCTACT | SEQ ID NO: 5392 |
| rs463302 | AACAAAGGCAAAAACGCGGACG | SEQ ID NO: 661 | AACAAAGGCAAAAACGCGGACC | SEQ ID NO: 3027 | TGGTTATGAAGGGTGTGGTC | SEQ ID NO: 5393 |
| rs1265100 | GTTAAAGCAGCAGCCTAGATTCAGAA | SEQ ID NO: 662 | GTTAAAGCAGCAGCCTAGATTCAGAG | SEQ ID NO: 3028 | TAAGAAAACATGATTTTTATTTTTC | SEQ ID NO: 5394 |
| rs3828875 | GCCAGGGGCTCTCCTCTCCT | SEQ ID NO: 663 | GCCAGGGGCTCTCCTCTCCC | SEQ ID NO: 3029 | CCAATTCTGCTAGAGTTGCCT | SEQ ID NO: 5395 |
| rs6917517 | CCTGCTCTGCTGGGGTCCAA | SEQ ID NO: 664 | CCTGCTCTGCTGGGGTCCAG | SEQ ID NO: 3030 | GTGAGAGTGTGAGCAACATG | SEQ ID NO: 5396 |
| rs3888777 | CACCAGGGGAAGCTTTCACAAA | SEQ ID NO: 665 | CACCAGGGGAAGCTTTCACAAG | SEQ ID NO: 3031 | AACAGAGAAACCCTCGAAGG | SEQ ID NO: 5397 |
| rs7769979 | GGTGGAATTGTGGGGACAAT | SEQ ID NO: 666 | GGTGGAATTGTTTGGGGACAAC | SEQ ID NO: 3032 | ATAGTGACTGATGTTTCAAATATATTC | SEQ ID NO: 5398 |
| rs3129931 | TTCTTTTGATGGTCTTAAGAAGGAGAAT | SEQ ID NO: 667 | TTCTTTTGATGGTCTTAAGAAGGAGAAC | SEQ ID NO: 3033 | AAAACCTAACTGAGAAAAATGC | SEQ ID NO: 5399 |
| rs3868086 | GCTCAGACTTTGACTTCCCCGAG | SEQ ID NO: 668 | GCTCAGACTTTGACTTCCCCGAC | SEQ ID NO: 3034 | TGATGAGCAATTTTCCATG | SEQ ID NO: 5400 |
| rs7773068 | GGCAAATGAACTAAATCTTTCCTGATAGT | SEQ ID NO: 669 | GGCAAATGAACTAAATCTTTCCTGATAGC | SEQ ID NO: 3035 | CTCCAGAATTTCCCCTATTAATA | SEQ ID NO: 5401 |
| rs3778638 | CCTGCTGCAGGCGGACACA | SEQ ID NO: 670 | CCTGCTGCAGGCGGACACG | SEQ ID NO: 3036 | TGCCTGGGTATTTAACATGAG | SEQ ID NO: 5402 |
| rs3130643 | AGAGATCTGGGGATTTCGGCTCT | SEQ ID NO: 671 | AGAGATCTGGGGATTTCGGCTCA | SEQ ID NO: 3037 | TGCTGACACTTCCTTCATG | SEQ ID NO: 5403 |
| rs3885028 | GCCAGGCTTGTCCACCCCT | SEQ ID NO: 672 | GCCAGGCTTGTCCACCCCA | SEQ ID NO: 3038 | GGGATCTCTCCTACCTGACTG | SEQ ID NO: 5404 |
| rs3757327 | CTTCAAGGAAGACTGGGAAATGGT | SEQ ID NO: 673 | CTTCAAGGAAGACTGGGAAATGGC | SEQ ID NO: 3039 | TAATAAAAATAGATACAATGTTATGGC | SEQ ID NO: 6405 |
| rs29225 | ACAGAGACACCCTCAAAGGACGAA | SEQ ID NO: 674 | ACAGAGACACCCTCAAAGGACGAG | SEQ ID NO: 3040 | AGTTCATTGCCCGGTAGATT | SEQ ID NO: 5406 |
| rs885937 | CCCTGAAGGCACAGGTACCCT | SEQ ID NO: 675 | CCCTGAAGGCACAGGTAACCCA | SEQ ID NO: 3041 | CACCCCATCTCCAACTATGA | SEQ ID NO: 5407 |
| rs3129702 | AGACTTGGGAGGTGATGTCATATAAA | SEQ ID NO: 676 | AGACTTGGGAGGTGATGTCATATAAC | SEQ ID NO: 3042 | GTTATTTAAAGCCTTGAGTCTGAA | SEQ ID NO: 5408 |
| rs3128968 | TGAGGGTGTCAGGAGATGAGGAAA | SEQ ID NO: 677 | TGAGGGTGTCAGGAGATGAGGAAT | SEQ ID NO: 3043 | TCCCTGGAGTCACAGAAAGA | SEQ ID NO: 5409 |
| rs4713327 | CCCTCATGGACCCCGGAT | SEQ ID NO: 678 | CCCTCATGGACCCCGGAC | SEQ ID NO: 3044 | TTTTGCAAGTCTGTCCTTACC | SEQ ID NO: 5410 |
| rs987870 | CACCTCCTTCAAAGCATCTGATTCTAT | SEQ ID NO: 679 | CACCTCCTTCAAAGCATCTGATTCTAC | SEQ ID NO: 3045 | TTGAAAGAGTGAATATTTTTACAAA | SEQ ID NO: 5411 |
| rs7772410 | CCTCCCACTTAAGAGTGAGGACACG | SEQ ID NO: 660 | CCTCCCACTTAAGAGTGAGGACACC | SEQ ID NO: 3046 | GGTGTTAGGTTTTCTGTTCC | SEQ ID NO: 5412 |
| rs3998790 | CTGTCTGTCACCACCCCCATTTT | SEQ ID NO: 681 | CTGTCTGTCACCACCCCCATTTC | SEQ ID NO: 3047 | CTGGTGACATCCCTCTTTC | SEQ ID NO: 5413 |
| rs733841 | CTGTCTGTCACCACCCCCATTTT | SEQ ID NO: 682 | CTGTCTGTCACCACCCCCATTTC | SEQ ID NO: 3048 | CTGGTGACATCCCTCTTTC | SEQ ID NO: 5414 |
| rs6916716 | GACCAGCCCCTTGTCAGGGA | SEQ ID NO: 683 | GACCAGCCCCTTGTCAGGGC | SEQ ID NO: 3049 | GCTGTCATCCCTGTTTTCAT | SEQ ID NO: 5415 |
| rs2395313 | TTGAGTTGCTTACTATGTTCTAGGCACT | SEQ ID NO: 684 | TTGAGTTGCTTACTATGTTCTAGGCACG | SEQ ID NO: 3050 | TTCTAAGTGCTTCTCATGTGTTC | SEQ ID NO: 5416 |
| rs7744001 | CAAGTGTTGGTGCCTCTTTGTGCA | SEQ ID NO: 685 | CAAGTGTTGGTGCCTCTTTGTGCG | SEQ ID NO: 3051 | CAGTGTCCCTGTCTGAGGAC | SEQ ID NO: 5417 |
| rs3757329 | GAAAGGAAGCAGGGATGAAGTGGT | SEQ ID NO: 686 | GAAAGGAAGCAGGGATGAAGTGGG | SEQ ID NO: 3052 | CACTTGAGAAGGGGTTCAG | SEQ ID NO: 5418 |
| rs6933135 | GCCTTGGACTGAGCCAGGCTA | SEQ ID NO: 687 | GCCTTGGACTGAGCCAGGCTG | SEQ ID NO: 3053 | TGTCTTCCTTGGTTCTCCAG | SEQ ID NO: .5419 |
| rs4713217 | GCATGAATCCTTAATATTGAAGAGACCAA | SEQ ID NO: 688 | GCATGAATCCTTAATATTGAAGAGACCAG | SEQ ID NO: 3054 | AGCTAAGCTTCTATACAACTTCTGA | SEQ ID NO: 5420 |
| rs3130198 | TTGGGAACGCACAGTTTTTCACT | SEQ ID NO: 689 | TTGGGAACGCACAGTTTTTCACC | SEQ ID NO: 3055 | GTCTGGTGGAGTTTGCAGT | SEQ ID NO: 5421 |
| rs5027459 | TGTTGCACAGGGACCAGGTATGA | SEQ ID NO: 690 | TGTTGCACAGGGACCAGGTATGG | SEQ ID NO: 3056 | TTGGGGCAGTTTAGAAGTG | SEQ ID NO: 5422 |
| rs2747430 | CGCTTGTTAGCACCCACTGAATA | SEQ ID NO: 691 | CGCTTGTTAGCACCCACTGAATG | SEQ ID NO: 3057 | GCTTATGTGCTCAGAAACGGT | SEQ ID NO: 5423 |
| rs4248158 | GGAGTGAAAGAGCCTCCAGGACT | SEQ ID NO: 692 | GGAGTGAAAGAGCCTCCAGGACC | SEQ ID NO: 3058 | CCAGGTATGGAATACAGGG | SEQ ID NO: 5424 |
| rs2301227 | CTAGGGTGATGTAGGGGATGCACT | SEQ ID NO: 693 | CTAGGGTGATGTAGGGGATGCACC | SEQ ID NO: 3059 | GGCTTAGGTGCAAAATTTG | SEQ ID NO: 5425 |
| rs3668 | GGGTGGCTTACTCCAGGCAACT | SEQ ID NO: 694 | GGGTGGCTTACTCCAGGCAACC | SEQ ID NO: 3060 | TTAGTAGGTAACACTTAAGAAGAAAAA | SEQ ID NO: 5426 |
| rs1233426 | CAAGGTCCGGCAGCTCTTCATA | SEQ ID NO: 695 | CAAGGTCCGGCAGCTCTTCATG | SEQ ID NO: 3061 | GGATGGACTCATCTCAGCAG | SEQ ID NO: 5427 |
| rs3094093 | GGTAAGTGCTGGCCTTCCTTCCTA | SEQ ID NO: 696 | GGTAAGTGCTGGCCTTCCTTCCTT | SEQ ID NO: 3062 | ACCTCTGAAATCAACCAGG | SEQ ID NO: 5428 |
| rs422951 | AGTGAGGGGGACCCTGGGT | SEQ ID NO: 697 | AGTGAGGGGGACCCTGGGC | SEQ ID NO: 3063 | TCACACTCATCCACATCTTC | SEQ ID NO: 5429 |
| rs909729 | TCTACAATGGCATTCAATAAAGTGCAA | SEQ ID NO: 698 | TCTACAATGGCATTCAATAAAGTGCAT | SEQ ID NO: 3064 | TGTTCCTGGTTAAAAAAAAAA | SEQ ID NO: 5430 |
| rs2534673 | GACAGACAACGCGTGGTTTTTCTC | SEQ ID NO: 699 | GACAGACAACGCGTGGTTTTTCTG | SEQ ID NO: 3065 | CAATACACTCCCTCAACCG | SEQ ID NO: 5431 |
| rs6928482 | AGGCCATGACCCTGAAAGCAGA | SEQ ID NO: 700 | AGGCCATGACCCTGAAAGCAGG | SEQ ID NO: 3066 | ATCCCATCTTCCTTTCTCC | SEQ ID NO: 5432 |
| rs707938 | GGCCCTTGTGGCAGCAGT | SEQ ID NO: 701 | GGCCCTTGTGGCAGCAGC | SEQ ID NO: 3067 | GTAGCTGAACAAGGCTCAGAA | SEQ ID NO: 5433 |
| rs2844521 | GGAGAGCTAGGTCCATCTCCCTCT | SEQ ID NO: 702 | GGAGAGCTAGGTCCATCTCCCTCC | SEQ ID NO: 3068 | AGTCTCTCACTAGGCTTATGATCT | SEQ ID NO: 5434 |
| rs3868075 | CCAGAACTCTACTGAAATCAAGTCTGGAT | SEQ ID NO: 703 | CCAGAACTCTACTGAAATCAAGTCTGGAC | SEQ ID NO: 3069 | AAAGTCAATAATATTCTTCTGACCG | SEQ ID NO: 5435 |
| rs7766843 | CAAGAGAAGAATGACAGCAAGCA | SEQ ID NO: 704 | CAAGAGAAGAATGACAGCAAGCG | SEQ ID NO: 3070 | AAGGGTTTTTCCCTTTATAAAAC | SEQ ID NO: 5436 |
| rs4151651 | CGATGCTGAGGATGGGCACA | SEQ ID NO: 705 | CGATGCTGAGGATGGGCACG | SEQ ID NO: 3071 | CCCAGGTTTGAAGACAGAGA | SEQ ID NO: 5437 |
| rs2394609 | CCCATATGAATTGATCAGAGAAGAATT | SEQ ID NO: 706 | CCCATATGAATTGATCAGAGAAGAATG | SEQ ID NO: 3072 | TGTTCTTCATAGACTACAAAATTCC | SEQ ID NO: 5438 |
| rs2239805 | CTGTCTTCTCCTACTATCCAGCTTCCT | SEQ ID NO: 707 | CTGTCTTCTCCTACTATCCAGCTTCCG | SEQ ID NO: 3073 | CTTTTTTTAATCTGTAATTCTCTCAA | SEQ ID NO: 5439 |
| rs5007260 | ACCTGTTACACTGCCTCCCACTTAT | SEQ ID NO: 708 | ACCTGTTACACTGCCTCCCACTTAC | SEQ ID NO: 3074 | TTTATATCCCACTGCGCAGT | SEQ ID NO: 5440 |
| rs2073044 | CCATCTAGTTATCCTTTCCTCCCCA | SEQ ID NO: 709 | CCATCTAGTTATCCTTTCCTCCCCG | SEQ ID NO: 3075 | TGAGTTATTTAACACTAGAAAAAAAA | SEQ ID NO: 5441 |
| rs3765608 | TCTCTGAGCAGGGTAGCAGGGAG | SEQ ID NO: 710 | TCTCTGAGCAGGGTAGCAGGGAC | SEQ ID NO: 3076 | TCTGGTCCTGATCTGAGTG | SEQ ID NO: 5442 |
| rs769176 | AAAAACCGATGCCCCACTTAGGA | SEQ ID NO: 711 | AAAAACCGATGCCCCACTTAGGG | SEQ ID NO: 3077 | GTCCCATTTAGGGTGAGAC | SEQ ID NO: 5443 |
| rs7773694 | GGATACAGGGGAAAAAAGATAGAAATGTA | SEQ ID NO: 712 | GGATACAGGGGAAAAAAGATAGAAATGTG | SEQ ID NO: 3078 | TATTTACTACACAGCAAAGAGGAT | SEQ ID NO: 5444 |
| rs3126935 | TCTCTAGACCCCCCTGCATGTTA | SEQ ID NO: 713 | TCTCTAGACCCCCCTGCATGTTG | SEQ ID NO: 3079 | CGGTGTTCTCTCTCTGTGG | SEQ ID NO: 5445 |
| rs2853950 | TCTCTCCCTTCCCTGATTCCAGTA | SEQ ID NO: 714 | TCTCTCCCTTCCCTGATTCCAGTG | SEQ ID NO: 3080 | TCTTCGTGCTGACTCCAAT | SEQ ID NO: 5446 |
| rs494620 | GCTGGGCGTGCTGGCATAT | SEQ ID NO: 715 | GCTGGGCGTGCTGGCATAC | SEQ ID NO: 3081 | GCATCTACTACTGCTGGGAG | SEQ ID NO: 5447 |
| rs1633089 | TACAACCTCACGGTGCTGTCCCA | SEQ ID NO: 716 | TACAACCTCACGGTGCTGTCCCG | SEQ ID NO: 3082 | GATGGATCTGTGCAGTCAG | SEQ ID NO: 5448 |
| rs2267642 | TCACCCAAACTCACTTCCCAAACT | SEQ ID NO: 717 | TCACCCAAACTCACTTCCCAAACC | SEQ ID NO: 3083 | CATCTCCTCACAAAGGCAG | SEQ ID NO: 5449 |
| rs2259571 | CAACCCCTTCCTCAGTCCCCT | SEQ ID NO: 718 | CAACCCCTTCCTCAGTCCCCG | SEQ ID NO: 3084 | CTGAAAACCCTCCAGTCAG | SEQ ID NO: 5450 |
| rs1557608 | CAGGGTCTCTCCTCAGGAGAGAAT | SEQ ID NO: 719 | CAGGGTCTCTCCTCAGGAGAGAAG | SEQ ID NO: 3085 | CAGTTATACTGAAGATGCCTTCC | SEQ ID NO: 5451 |
| rs2530708 | CGCAGTCTGATTTTTACTGTCTCACATAGT | SEQ ID NO: 720 | CGCAGTCTGATTTTTACTGTCTCACATAGC | SEQ ID NO: 3086 | TAGTATAAAGACCAGGGAGAAGG | SEQ ID NO: 5452 |
| rs7769930 | TCCCACCAGCACCCAATCCT | SEQ ID NO: 721 | TCCCACCAGCACCCAATCCG | SEQ ID NO: 3087 | GGATATTTATTCTCTTTGTCTCACA | SEQ ID NO: 5453 |
| rs2097274 | AAAGTCACATTCAAATGCACAAATGT | SEQ ID NO: 722 | AAAGTCACATTCAAATGCACAAATGC | SEQ ID NO: 3088 | CTAAGAAATTTTTAGCAATAAGATTT | SEQ ID NO: 5454 |
| rs422878 | CGCAAAGAAATAGCACTCAGACATAAATTT | SEQ ID NO: 723 | CGCAAAGAAATAGCACTCAGACATAAATTC | SEQ ID NO: 3089 | ATTTTCTCAGCAAGGAATTTTTA | SEQ ID NO: 5455 |
| rs6457643 | AATGTGAAAAAATGTTTCATTTCCAGA | SEQ ID NO: 724 | AATGTGAAAAAATGTTTCATTTCCAGC | SEQ ID NO: 3090 | TTTATTTCTTTGTATAGATCCAGGT | SEQ ID NO: 5456 |
| rs7381617 | CATGCTCATTGCTGATACATAGAAAAA | SEQ ID NO: 725 | CATGCTCATTGCTGATACATAGAAAAG | SEQ ID NO: 3091 | GATTGGCTTTTACATATTAACTTT | SEQ ID NO: 5457 |
| rs3129270 | AAAAAAAGTGATGAGACATGACTTGGTA | SEQ ID NO: 726 | AAAAAAAGTGATGAGACATGACTTGGTG | SEQ ID NO: 3092 | ATATTCTTAACAGAAAACAATCATG | SEQ ID NO: 5458 |
| rs1632914 | ACTGGGCAGCAGCCCAGGGA | SEQ ID NO: 727 | ACTGGGCAGCAGCCCAGGGG | SEQ ID NO: 3093 | GCTTTACAGGTTTGTGTTTTACA | SEQ ID NO: 5459 |
| rs3129808 | TTTCCATGTTAATCAATCAAGTCCATA | SEQ ID NO: 728 | TTTGCATGTTAATCAATCAAGTCCATC | SEQ ID NO: 3094 | ATTGTTTATATGTGTAGCTTTCTCTC | SEQ ID NO: 5460 |
| rs4713604 | TGCAAAGCTAGGAGTTCATGAAGTGG | SEQ ID NO: 729 | TGCAAAGCTAGGAGTTCATGAAGTGC | SEQ ID NO: 3095 | TTTCAATAATAGAGATGCAAAG | SEQ ID NO: 5461 |
| rs241436 | ATAGTCCTCTGCCCCTGTCCCT | SEQ ID NO: 730 | ATAGTCCTCTGCCCCTGTCCCC | SEQ ID NO: 3096 | CTGCACAGGTGGTTTCAGTT | SEQ ID NO: 5462 |
| rs3132555 | TTGTCTTCCTCCTCTGTGGGAGG | SEQ ID NO: 731 | TTGTCTTCCTCCTCTGTGGGAGC | SEQ ID NO: 3097 | GCTGGAAGGTAGAAGAGAAA | SEQ ID NO: 5483 |
| rs2517461 | ACACTTGAAATATGGCAAGTACGACTA | SEQ ID NO: 732 | ACACTTGAAATATGGCAAGTACGACTG | SEQ ID NO: 3098 | GAAACTGAACGTTTTTATTTTATT | SEQ ID NO: 5464 |
| rs5021448 | GCAGCCCCCAGGTATCAGGAT | SEQ ID NO: 733 | GCAGCCCCCAGGTATCAGGAC | SEQ ID NO: 3099 | CTTTTTTATTTTACCTTTTTTTAGTC | SEQ ID NO: 5465 |
| rs4122198 | GAGAAGCTGCAGTGAGTTTTATGTTCTA | SEQ ID NO: 734 | GAGAAGCTGCAGTGAGTTTTATGTTCTG | SEQ ID NO: 3100 | ACCCAGAGTCAAAAACTGTTATAT | SEQ ID NO: 5466 |
| rs2227134 | TTCAGCTTTGACAATATTGGGGGA | SEQ ID NO: 735 | TTCAGCAAGACAATATTGGGGGG | SEQ ID NO: 3101 | AAAAAAAGATGTAGATACTTAATTTTG | SEQ ID NO: 5467 |
| rs4713605 | TTGCCAAGGTCACCAAATCTTTAA | SEQ ID NO: 736 | TTGCCAAGGTCACCAAATCTTTAT | SEQ ID NO: 3102 | TTTGTGGAGTTTTCTGCTG | SEQ ID NO: 5468 |
| rs1265158 | AGGCCGAGAAGTGAACACAGCTC | SEQ ID NO: 737 | AGGCCGAGAAGTGAACACAGCTG | SEQ ID NO: 3103 | AACCCCACTGCCTTGTAGAC | SEQ ID NO: 5469 |
| rs3135382 | ACTCTGAAATCGCGGTGCAGTA | SEQ ID NO: 738 | ACTCTGAAATCGCGGTGCAGTC | SEQ ID NO: 3104 | TAAAATCTTGGAGGCGGTT | SEQ ID NO: 5470 |
| rs3129701 | AGCGGCATCTCAGATCCCACA | SEQ ID NO: 739 | AGCGGCATCTCAGATCCCACG | SEQ ID NO: 3105 | CTCCCATAGTGACTGCCTG | SEQ ID NO: 5471 |
| rs2244548 | AAGACTTGGGAACACAACTCAAGG | SEQ ID NO: 740 | TTTGACTTGGGAACACTTTCTCAAGC | SEQ ID NO: 3106 | ATGATGTATTAGTCCATTTCATGTT | SEQ ID NO: 5472 |
| rs2395175 | AAGATCAGGAACCCACAGGAAGCT | SEQ ID NO: 741 | AAGATCAGGAACCCACAGGAAGCC | SEQ ID NO: 3107 | CTGCTGATGTTGCAGATCCT | SEQ ID NO: 5473 |
| rs2844793 | CATTCTGCAGAAACCTGTCACCATT | SEQ ID NO: 742 | CATTCTGCAGAAACCTGTCACCATC | SEQ ID NO: 3108 | ACTGTGGGCACAATTTCTG | SEQ ID NO: 5474 |
| rs2395327 | GATCACATGCTTCAGAGGGCAA | SEQ ID NO: 743 | GATCACATGCTTCAGAGGGCAG | SEQ ID NO: 3109 | AAAAGATCACAAGGCAGAGG | SEQ ID NO: 5475 |
| rs1265074 | CCCCATCCACCTCAAAGTGCA | SEQ ID NO: 744 | CCCCATCCACCTCAAAGTGCC | SEQ ID NO: 3110 | AAACTTCACCTCTCCTGGC | SEQ ID NO: 5476 |
| rs3117228 | GGGCCTGACTCTTAGAAAACACTCTATA | SEQ ID NO: 745 | GGGCCTGACTCTTAGAAAACACTCTATC | SEQ ID NO: 3111 | ATATTGAAAATTTGAAGTCTAGTGA | SEQ ID NO: 5477 |
| rs429479 | TGATCAAGGGACTAGAACACTAAACTGGA | SEQ ID NO: 746 | TGATCAAGGGACTAGAACACTAAACTGGG | SEQ ID NO: 3112 | AAATGAGAATATATTAACTAGGGGA | SEQ ID NO: 5478 |
| rs1634720 | GGCACATCTTGATCTGGAAAGT | SEQ ID NO: 747 | GGCACATCTTGATCTGGAAAGC | SEQ ID NO: 3113 | GGTAGGTGAGTGTATGCATGT | SEQ ID NO: 5479 |
| rs3095302 | CTTGGCTCCAGCGAGAGGCCA | SEQ ID NO: 748 | CTTGGCTCCAGCGAGAGGCCG | SEQ ID NO: 3114 | TTCCTCTCCTCTCTCTTTCTC | SEQ ID NO: 5480 |
| rs423023 | GTGTGTGTGAGTGGCTGGGGG | SEQ ID NO: 749 | GTGTGTGTGAGTGGCTGGGGC | SEQ ID NO: 3115 | GCACAAGCTGTGAGGAGAAC | SEQ ID NO: 5481 |
| rs6456880 | CTGATGGATGGTAAGGCAGTGCTT | SEQ ID NO: 750 | CTGATGGATGGTAAGGCAGTGCTG | SEQ ID NO: 3116 | TCTTGGAAGGTTTTCCCAC | SEQ ID NO: 5482 |
| rs1065461 | GTCCGATGGTGCCTTGGCA | SEQ ID NO: 751 | GTCCGATGGTGCCTTGGCG | SEQ ID NO: 3117 | TGGACCTTAGGTCTGAATGC | SEQ ID NO: 5483 |
| rs6923313 | GCCACTAGGAGAATTAAAACCATAAGCTA | SEQ ID NO: 752 | GCCACTAGGAGAATTAAAACCATAAGCTG | SEQ ID NO: 3118 | AAAATGTTTACTTGACACATAAGAA | SEQ ID NO: 5484 |
| rs1367730 | TCAAAATGCTGCATAGAATGTCAGAT | SEQ ID NO: 753 | TCAAAATGCTGCATAGAATGTCAGAC | SEQ ID NO: 3119 | GTTAAACAATTTTGTTAGAGTAAAATG | SEQ ID NO: 5485 |
| rs1800629 | GCAATAGGTTTTGAGGGGCATGA | SEQ ID NO: 754 | GCAATAGGTTTTGAGGGGCATGG | SEQ ID NO: 3120 | GTCCTACACACAAATCAGTCAGT | SEQ ID NO: 5486 |
| rs2240070 | TTCCCCTCCTTTTGCTCAAGAAT | SEQ ID NO: 755 | TTCCCCTCCTTTTGCTCAAGAAC | SEQ ID NO: 3121 | CGTCCATTCCTTCTCCAACT | SEQ ID NO: 5487 |
| rs520803 | CAAGTGCCCCCATTCTGACAT | SEQ ID NO: 756 | CAAGTGCCCCCATTCTGACAC | SEQ ID NO: 3122 | GGTGGCTGACACAGTTGTCT | SEQ ID NO: 5488 |
| rs3734838 | AGTGCAGGGGGGTAAAAATTAATAACT | SEQ ID NO: 757 | AGTGCAGGGGGGTAAAAATAAATAACC | SEQ ID NO: 3123 | AATAAATAATCATGGGCTGTTTAC | SEQ ID NO: 5409 |
| rs3132468 | GCCCCAGCCCTGGCTTGA | SEQ ID NO: 758 | GCCCCAGCCCTGGCTTGG | SEQ ID NO: 3124 | AAGCACTTATGGGTGTAGATG | SEQ ID NO: 5490 |
| rs3094159 | GTTTTGCCAGGTGAGTGTGACAAT | SEQ ID NO: 759 | GTTTTGCCAGGTGAGTGTGACAAC | SEQ ID NO: 3125 | GGGATGGAAATTATTTACCATAG | SEQ ID NO: 5491 |
| rs3160665 | CTTGTGTGCTTGCTGGGATCAAA | SEQ ID NO: 760 | CTTGTGTGCTTGCTGGGATCATG | SEQ ID NO: 3126 | CAACCTGCTTACTTTTTCTCTTT | SEQ ID NO: 5492 |
| rs2518027 | GACCTGAGAAGATGCCCTGGTTA | SEQ ID NO: 761 | GACCTGAGAAGATGCCCTGGTTC | SEQ ID NO: 3127 | TGTGATAGAAGCCTCTACACAAT | SEQ ID NO: 5493 |
| rs1362068 | AATGTGCATCATGAGAAAAGAAATGT | SEQ ID NO: 762 | AATGTGCATCATGAGAAAAGAAATGC | SEQ ID NO: 3128 | GTAGGAATTTCTGTACAGGAAG | SEQ ID NO: 5494 |
| rs1264542 | AACCAGGTGTTGATCTGGGCTTT | SEQ ID NO: 763 | AACCAGGTGTTGATCTGGGCTTC | SEQ ID NO: 3129 | TAGGCTCATGTGCTCTGGAG | SEQ ID NO: 5495 |
| rs2223638 | TGCGCTGCACTTAACATTTGCTA | SEQ ID NO: 764 | TGCGCTGCACTTAACATTTGCTG | SEQ ID NO: 3130 | TTAAAACAAAAAAATTTTGGAA | SEQ ID NO: 5496 |
| rs2281390 | CATGGTCTCAGGCACCTGGATTAT | SEQ ID NO: 765 | CATGGTCTCAGGCACCTGGATTAG | SEQ ID NO: 3131 | ACCAAGGGAAGACAGAGTTATAA | SEQ ID NO: 5497 |
| rs1265094 | TATAGGGGAGGAGTGGAGGAGGGA | SEQ ID NO: 766 | TATAGGGGAGGAGTGGAGGAGGGG | SEQ ID NO: 3132 | AGGAATACCATCAGAACAGAACT | SEQ ID NO: 5498 |
| rs2523994 | CAGAGAAGTCTTAGGCCCGGCA | SEQ ID NO: 767 | CAGAGAAGTCTTAGGCCCGGCC | SEQ ID NO: 3133 | TAATCCCTAAATTTCACAATGTG | SEQ ID NO: 5499 |
| rs2857129 | CCAGCAGGCCTTCACCAGAA | SEQ ID NO: 766 | CCAGCAGGCCTTCACCAGAT | SEQ ID NO: 3134 | CCTACCAGGCTGACACACT | SEQ ID NO: 5500 |
| rs885403 | AGTGATGTCTGAGGTGGCCACAA | SEQ ID NO: 769 | AGTGATGTCTGAGGTGGCCACAG | SEQ ID NO: 3135 | TCTCCAAAAAATATCCTTTCTAGA | SEQ ID NO: 5501 |
| rs2213565 | CTACCGCTGCCACCAATGGTAT | SEQ ID NO: 770 | CTACOGCTGCCACCAATGGTAC | SEQ ID NO: 3136 | CGCCTCTCTTTACTGAAACTAAT | SEQ ID NO: 5502 |
| rs1419658 | CTGGTGCTGGGGGTGACAGT | SEQ ID NO: 771 | CTGGTGCTGGGGGTGACAGC | SEQ ID NO: 3137 | TGGGGAAAGATGCTAGATG | SEQ ID NO: 5503 |
| rs2022109 | GCAAGAGGATCACCTAGGAGAGTAAATTAC | SEQ ID NO: 772 | GCAAGAGGATCACCTAGGAGAGTAAATTAG | SEQ ID NO: 3136 | AAAAGATGGCAGTATTAGCAATC | SEQ ID NO: 5504 |
| rs3128928 | CAGACACCAGGAGGAAAGGGGTT | SEQ ID NO: 773 | CAGACACCAGGAGGAAAGGGGTG | SEQ ID NO: 3139 | CCTCTCACTCTGTGTGTATTTCT | SEQ ID NO: 5505 |
| rs707913 | GCCACTGCTGGATACCCCAA | SEQ ID NO: 774 | GCCACTGCTGGATACCCCAG | SEQ ID NO: 3140 | GGTTTGGGAAGAGGAAGAG | SEQ ID NO: 5506 |
| rs2027856 | GGCTCTTCAGATTCCTTCATGGCT | SEQ ID NO: 775 | GGCTCTTCAGATTCCTTCATGGCC | SEQ ID NO: 3141 | ACTAAAACATTCTTTGTGTCCAC | SEQ ID NO: 5507 |
| rs2747454 | TGATGTCAGGTTCCAGGGTCAGT | SEQ ID NO: 776 | TGATGTCAGGTTCCAGGGTCAGC | SEQ ID NO: 3142 | ACCTCTGCCCAACTCAGAC | SEQ ID NO: 5508 |
| rs3132552 | GATACGCGTGGGGTCCTTACAA | SEQ ID NO: 777 | GATACGCGTGGGGTCCTTACAG | SEQ ID NO: 3143 | GGTCTGAGAAGGTGCCAAT | SEQ ID NO: 5509 |
| rs3130989 | GGTAAGTTGGGCCTGGGGTTTTC | SEQ ID NO: 778 | GGTAAGTTGGGCCTGGGGTTTTG | SEQ ID NO: 3144 | AAGGAAAGATAAGGAAAGATGTG | SEQ ID NO: 5510 |
| rs3017964 | CCTTCAACCCCATTAGGCCATAAA | SEQ ID NO: 779 | CCTTCAACCCCATTAGGCCATAAT | SEQ ID NO: 3145 | ATTAAGGGAAGGGTCCCTG | SEQ ID NO: 5511 |
| rs154971 | GAATGGAACGGTGGCTCTGAAA | SEQ ID NO: 760 | GAATGGAACGGTGGCTCTGAAG | SEQ ID NO: 3146 | TGAGGGTCTTCAGAATAAAGTAA | SEQ ID NO: 5512 |
| rs4713326 | CTCCTGGCCTCATGACCCAGG | SEQ ID NO: 781 | CTCCTGGCCTCATGACCCAGC | SEQ ID NO: 3147 | GTCCAATCCTGACATCCCT | SEQ ID NO: 5513 |
| rs3132499 | GCATTTTACCCCTTAGTACTGGGGCA | SEQ ID NO: 782 | GCATTTACCCCTTAGTACTGGGGCG | SEQ ID NO: 3148 | TCTCAGTAAGGATGAAATGTGT | SEQ ID NO: 5514 |
| rs2395318 | CATTTTGTGTAGTGCTGGATTGGTAGT | SEQ ID NO: 783 | CATTTTGTGTAGTGCTGGATTGGTAGC | SEQ ID NO: 3149 | GTGAAGTCTCTCAGCATTTGTT | SEQ ID NO: 5515 |
| rs3823343 | GTGTTGGGCGGAACAGTGGACA | SEQ ID NO: 784 | GTGTTGGGCGGAACAGTGGACG | SEQ ID NO: 3150 | AGCTGTGCTATGGGGTTTCT | SEQ ID NO: 5516 |
| rs2071463 | CCAGCAACTGTCGACGTCACAT | SEQ ID NO: 785 | CCAGCAACTGTCGACGTCACAC | SEQ ID NO: 3151 | AATGGGAGATATTTTACTAGGCAA | SEQ ID NO: 5517 |
| rs2857770 | GCAAGGAGGGTATGGGGAATGTTT | SEQ ID NO: 786 | GCAAGGAGGGTATGGGGAATGTTC | SEQ ID NO: 3152 | ATCAACAACAAAGTCCTTTCTCT | SEQ ID NO: 5518 |
| rs4248154 | GAAATGGGACATGGAGGAACACAT | SEQ ID NO: 787 | GAAATGGGACATGGAGGAACACAC | SEQ ID NO: 3153 | GCTTTGGATATGGAGTGGG | SEQ ID NO: 5519 |
| rs3132595 | GAGAATGTAAGAGGGGCAAGGGTC | SEQ ID NO: 788 | GAGAATGTAAGAGGGGCAAGGGTG | SEQ ID NO: 3154 | GTACCTTAACACAAGGGAAGAGA | SEQ ID NO: 5520 |
| rs3749970 | CTGCGACTGCACACGAGGTTTACA | SEQ ID NO: 789 | CTGCGACTGCACACGAGGAACG | SEQ ID NO: 3155 | GCAGTGTCACTGAAGAATAAGAA | SEQ ID NO: 5521 |
| rs541862 | CTCAACTTGCTCACCCTGCCAT | SEQ ID NO: 790 | CTCAACTTGCTCACCCTGCCAC | SEQ ID NO: 3156 | TGTATCCCTGCCTTTAGCC | SEQ ID NO: 5522 |
| rs2075582 | GGCACTGGGGGAAACTTAGGCA | SEQ ID NO: 791 | GGCACTGGGGGAAACTTAGGCG | SEQ ID NO: 3157 | CTCCTCCAAGGCTCTCTTG | SEQ ID NO: 5523 |
| rs2854048 | CAGCCTCGGACTCCATCTCTCA | SEQ ID NO: 792 | CAGCCTCGGACTCCATCTCTCG | SEQ ID NO: 3158 | AAGCAAGACAACAGGGGTC | SEQ ID NO: 5524 |
| rs1557607 | CTTGGTCCCTTCTGACGTCACCA | SEQ ID NO: 793 | CTTGGTCCCTTCTGACGTCACCG | SEQ ID NO: 3159 | GGAGTCTGAACAGCAGATAATT | SEQ ID NO: 5525 |
| rs1362070 | GCTTTGCGGGTTCTGTGTCTTAT | SEQ ID NO: 794 | GCTTTGCGGGTTCTGTGTCTTAC | SEQ ID NO: 3160 | GGAACCCAGGTAACTACTAGATT | SEQ ID NO: 5526 |
| rs2621337 | CCTACCAGGCTGACACACTGATCTT | SEQ ID NO: 795 | CCTACCAGGCTGACACACTGATCTC | SEQ ID NO: 3161 | TGAAAAATACATTTCTGCTGTTT | SEQ ID NO: 5527 |
| rs3807030 | CAAGGGGAACAAATGCATACTCAGT | SEQ ID NO: 796 | CAAGGGGAACAAATGCATACTCAGG | SEQ ID NO: 3162 | TAGTGATCAAGGGGTTACTGTAA | SEQ ID NO: 5528 |
| rs2596502 | GTGTGAGGGGTCCTTGTGCTGT | SEQ ID NO: 797 | GTGTGAGGGGTCCTTGTGCTGC | SEQ ID NO: 3163 | GCCTGAGGCTATGTTAATAAAGA | SEQ ID NO: 5529 |
| rs2076530 | GCCAGTTTGGATCTGAAGGTGGTAA | SEQ ID NO: 798 | GCCAGTTTGGATCTGAAGGTGGTAG | SEQ ID NO: 3164 | ATTTTGTATTCAATACCTGCTCC | SEQ ID NO: 5530 |
| rs885932 | GCAAACCTCCTGAGTGGGATTCT | SEQ ID NO: 799 | GCAAACCTCCTGAGTGGGATTCC | SEQ ID NO: 3165 | GTGAGGATTGTGTAGCATCTTC | SEQ ID NO: 5531 |
| rs2517598 | GAGGCACCAGGAGATGTTCCAT | SEQ ID NO: 800 | GAGGCACCAGGAGATGTTCCAC | SEQ ID NO: 3166 | GAAGTTCCTCTGTTTTGTGTGT | SEQ ID NO: 5532 |
| rs2524079 | CCCCTCCCAGTCACTCCTGAA | SEQ ID NO: 801 | CCCCTCCCAGTCACTCCTGAG | SEQ ID NO: 3167 | AGAGAACAGTTTCTTTTCCCTAA | SEQ ID NO: 5533 |
| rs4286818 | GTGCTCCTGCTCCTGTGGGTA | SEQ ID NO: 802 | GTGCTCCTGCTCCTGTGGGTG | SEQ ID NO: 3168 | GAGAGAGTGAGTTGTTTCTGTTG | SEQ ID NO: 5534 |
| rs4151650 | GGCAGAGCCCCGGAGAGTA | SEQ ID NO: 803 | GGCAGAGCCCCGGAGAGTG | SEQ ID NO: 3169 | AACCGTCATAGCAGTGGAAA | SEQ ID NO: 5535 |
| rs2523933 | TTTCTGCTTAGCTGGCAGGACTCA | SEQ ID NO: 804 | TTTCTGCTTAGCTGGCAGGACTCC | SEQ ID NO: 3170 | CTGTCTCTCCCAAATGATTATG | SEQ ID NO: 5536 |
| rs194675 | AAAAGATGGCAGTATTAGCAATCTCT | SEQ ID NO: 805 | AAAAGATGGCAGTATTAGCAATCTCA | SEQ ID NO: 3171 | TAGTTTGACTACATTCATTCCAAA | SEQ ID NO: 5537 |
| rs3130615 | CCCAGCTTCCATCCCCTGTT | SEQ ID NO: 806 | CCCAGCTTCCATCCCCTGTC | SEQ ID NO: 3172 | CAGCAGCCTTGTCACCTAC | SEQ ID NO: 5538 |
| rs2050188 | CAGAGATTTTTAAGGCCAGAGATTGTAT | SEQ ID NO: 807 | CAGAGATTTTTAAGGCCAGAGATTGTAC | SEQ ID NO: 3173 | TCATTAATCTTTGTAACTCTTTTTTT | SEQ ID NO: 5539 |
| rs2395173 | GCTCTGCCCCAGGTTTCTGAACA | SEQ ID NO: 808 | GCTCTGCCCCAGGTTTCTGAACG | SEQ ID NO: 3174 | ACTCAGTATTATGAGATTTGGGAG | SEQ ID NO: 5540 |
| rs1573299 | CCGAGCTCATCAGCAGTGACAT | SEQ ID NO: 809 | CCGAGCTCATCAGCAGTGACAC | SEQ ID NO: 3175 | CTGAGTTCCTGCAGTACTTGTT | SEQ ID NO: 5541 |
| rs417812 | GGAGTCCCCATGAGCCTGTCT | SEQ ID NO: 810 | GGAGTCCCCATGAGCOTGTCC | SEQ ID NO: 3176 | CTTCAGAACTGTTGTTAGCTGAG | SEQ ID NO: 5542 |
| rs1633029 | CCGCCACAGTGCTAGGATTGT | SEQ ID NO: 811 | CCGCCAGAGTGCTAGGATTGG | SEQ ID NO: 3177 | CACCTGGCATGAATTAATTATAA | SEQ ID NO: 5543 |
| rs7454158 | AAAAAAAGCAAAAGCCAGTGACT | SEQ ID NO: 812 | AAAAAAGCAAAAGCCAGTGAGC | SEQ ID NO: 3178 | TGATGAAGCAATAAAATCATTAGT | SEQ ID NO: 5544 |
| rs2516670 | TTCTGTCTTCTCAAGCTTGATCTTTACTT | SEQ ID NO: 813 | TTCTGTCTTCTCAAGCTTGATCTTTACTC | SEQ ID NO: 3179 | AGTCTTCTTCATTGTCACAGTATC | SEQ ID NO: 5545 |
| rs4151652 | TGATCATTCCAGCCCCTCTGAAT | SEQ ID NO: 814 | TGATCATTCCAGCCCCTCTGAAC | SEQ ID NO: 3180 | GAAAATCTCCAGGTCCTATTCT | SEQ ID NO: 5546 |
| rs5026741 | TGGAGACTACAGGCAGGAAACCAT | SEQ ID NO: 815 | TGGAGACTACAGGCAGGAAACCAC | SEQ ID NO: 3181 | TGCTAGAGATGAGACTTTGCTAT | SEQ ID NO: 5547 |
| rs2158288 | TTTGCTTTTTGATTCAAAAATTTCACT | SEQ ID NO: 816 | TTTGCTTTTTGATTCAAAAATTTCACA | SEQ ID NO: 3182 | TAGTAAACAGAGCTATTTGTTCCTG | SEQ ID NO: 5548 |
| rs1051790 | GGGAGCTCTTCCTCTCCCAAAACG | SEQ ID NO: 817 | GGGAGCTCTTCCTCTCCCAAAACC | SEQ ID NO: 3183 | GGAGACTGAGGAATGGACA | SEQ ID NO: 5549 |
| rs3828799 | GGTCTCTCTGCCTACTCTTCCTTGCG | SEQ ID NO: 818 | GGTCTCTCTGCCTACTCTTCCTTGCC | SEQ ID NO: 3184 | CAACTCCATTCCAGGTGTG | SEQ ID NO: 5550 |
| rs1737005 | GTGGTCCATGACCATGGCAT | SEQ ID NO: 819 | GTGGTCCATGACCATGGCAC | SEQ ID NO: 3185 | CCCTGGTCTTATCACCACCT | SEQ ID NO: 5551 |
| rs3132946 | GTGCACCATCCTGATGCCTGTT | SEQ ID NO: 820 | GTGCACCATCCTGATGCCTGTC | SEQ ID NO: 3186 | TAGCCCCTTTAATCTCCCC | SEQ ID NO: 5552 |
| rs3132467 | AGGCACTTTCCAGGTTCCCACT | SEQ ID NO: 821 | AGGCACTTTCCAGGTTCCCACC | SEQ ID NO: 3187 | TGCAGATGACACAGATGAATAG | SEQ ID NO: 5553 |
| rs2076542 | CCTGACTTGGGGATGCCTCAA | SEQ ID NO: 822 | CCTGACTTGGGGATGCCTCAG | SEQ ID NO: 3188 | GCCATGTAAAGCTTTAAAATTCT | SEQ ID NO: 5554 |
| rs3828882 | ACTTGCAGGTCAGGGGTCCCA | SEQ ID NO: 823 | ACTTGCAGGTCAGGGGTCCCG | SEQ ID NO: 3189 | GTGAGAACAGTGAAGAGAAACAG | SEQ ID NO: 5555 |
| rs3094717 | CAGTGCAGGTTTGTGGTCGCTC | SEQ ID NO: 824 | CAGTGCAGGTTTGTGGTCGTTG | SEQ ID NO: 3190 | ACCTGCACTGTCTCCTATAGAG | SEQ ID NO: 5556 |
| rs3094214 | GAGAAACCCGAGAGGCCGATT | SEQ ID NO: 825 | GAGAAACCCGAGAGGCCGATG | SEQ ID NO: 3191 | CTGAGATAAGGCAGAAAGGTG | SEQ ID NO: 5557 |
| rs2734984 | GTTTCCATGCTGGGCCTGCCA | SEQ ID NO: 826 | GTTTCCATGCTGGGCCTGCCG | SEQ ID NO: 3192 | GCTGATGGGATTCTTTAATGG | SEQ ID NO: 5558 |
| rs3131018 | AAAGCCAGCCAGAACCAAGCAT | SEQ ID NO: 827 | AAAGCCAGCCAGAACCAAGCAG | SEQ ID NO: 3193 | GCTGCAGAAGGAGAGAAGG | SEQ ID NO: 5559 |
| rs419957 | CTTTGTGGCCATCAGCTTCCTC | SEQ ID NO: 828 | CTTTGTGGCCATCAGCTTCCTG | SEQ ID NO: 3194 | CCTTAGTTGTCCTTGTGTACCT | SEQ ID NO: 5560 |
| rs1737075 | AGGAGTTGAGGCCGGGGAGA | SEQ ID NO: 829 | AGGAGTTGAGGCCGGGGAGC | SEQ ID NO: 3195 | GCTTCAAGCTGTAGGGGAT | SEQ ID NO: 5561 |
| rs3765607 | GGGAGCACAGAGCCCAGACACT | SEQ ID NO: 830 | GGGAGCACAGAGCCCAGACACC | SEQ ID NO: 3196 | GATAATCTCCTATTCCTTGGAAA | SEQ ID NO: 5562 |
| rs1800683 | AGGAGAGCCTCACCTGCTGTGT | SEQ ID NO: 831 | AGGAGAGCCTCACCTGCTGTGC | SEQ ID NO: 3197 | CTCAGGTCCCTTTATAGAGGAA | SEQ ID NO: 5563 |
| rs3763315 | CATAAATATCATACTGCCCTTTTCGATA | SEQ ID NO: 832 | CATAAATATGATACTGCCCTTTTCGATC | SEQ ID NO: 3198 | AGGTATTGTGAATGGGCTCATA | SEQ ID NO: 5564 |
| rs2187824 | GGAAGCCCTGGTGTGGGAAATTAT | SEQ ID NO: 833 | GGAAGCCCTGGTGTGGGAAATTAC | SEQ ID NO: 3199 | GACAGAAAGAAGGCTAGAGAGAC | SEQ ID NO: 5565 |
| rs6457041 | GGACCACGCTTCATCCCCTTT | SEQ ID NO: 834 | GGACGACGCTTCATCCCCTTC | SEQ ID NO: 3200 | GCAGATAATGTACTGAAATTCTGA | SEQ ID NO: 5566 |
| rs2285797 | CACAGCTAAAGCCAATAAAAATAAACAA | SEQ ID NO: 835 | CACAGCTAAAGCCAATAAAAATAAACAG | SEQ ID NO: 3201 | AATAGGATCTACTTTTCTGGAATC | SEQ ID NO: 5567 |
| rs2621336 | TGGAGAAGACAGCTATTTTCCATAAAAGTA | SEQ ID NO: 836 | TGGAGAAGACAGCTATTTTCCATATAAGTG | SEQ ID NO: 3202 | GTTGAAATGTTAACATGTAATGGG | SEQ ID NO: 5568 |
| rs4713614 | CAGCTCCAAGGGTGTGACAGGA | SEQ ID NO: 837 | CAGCTCCAAGGGTGTGACAGGC | SEQ ID NO: 3203 | CTCCCTCTATACATGATCTCCC | SEQ ID NO: 5569 |
| rs4428528 | TTAAGAATATGACTTGGCGACTGGG | SEQ ID NO: 838 | TTAAGAATATGACTTGGCGACTGGC | SEQ ID NO: 3204 | AGGCAAATAAATCAGTAAACCTT | SEQ ID NO: 5570 |
| rs2071365 | CCAGTAAGAATGACTCAGAGTCTCCCT | SEQ ID NO: 839 | CCAGTAAGAATGACTCAGAGTCTCCCC | SEQ ID NO: 3205 | CTCTGAAATACTGAAGTATTTATTGG | SEQ ID NO: 5571 |
| rs2246638 | AGAGAGCCAAGGGCCCCACA | SEQ ID NO: 840 | AGAGAGCCAAGGGCCCCACG | SEQ ID NO: 3206 | TGGAAAGCATCAGAGAGGA | SEQ ID NO: 5572 |
| rs1264697 | TGCTTCTCACAACTGCTTAAATTAGGTATC | SEQ ID NO: 841 | TGTTTCTCACAACTGGTTAAATTAGGTATG | SEQ ID NO: 3207 | TTACATTCCCATATTCCAAATAT | SEQ ID NO: 5573 |
| rs2280773 | AACTGGGACTGCAGCTGGGAT | SEQ ID NO: 842 | AACTGGGACTGCAGCTGGGAC | SEQ ID NO: 3208 | GTCACAGAAGTCACTGAGATCAG | SEQ ID NO: 5574 |
| rs4248169 | AAAACTGCCTTATAACACCATTCAAGA | SEQ ID NO: 843 | AAAACTGCCTTATAACACCATTCAAGC | SEQ ID NO: 3209 | TTGTTTTTATTTTCAGCAAACAC | SEQ ID NO: 5575 |
| rs1632941 | ACCAGAGGGAGGGCGATATTCTA | SEQ ID NO: 844 | ACCAGAGGGAGGGCGATATTCTG | SEQ ID NO: 3210 | TGTTGGTCCCATTTTCCTC | SEQ ID NO: 5576 |
| rs1610653 | GTACACATGCCATGTGCAGCAT | SEQ ID NO: 845 | GTACACATGCCATGTGCAGCAC | SEQ ID NO: 3211 | CACTAAGATGGGGTAAGGAGAC | SEQ ID NO: 5577 |
| rs6911894 | CCAAATATACTGAAATAATGATGTGCTGT | SEQ ID NO: 846 | CCAAATATACTGAAATAATGATGTGCTGC | SEQ ID NO: 3212 | GTCTTTTCATCTGTTAATATGGTG | SEQ ID NO: 5578 |
| rs3778614 | CTCAGGCCCTCCTCTCCCCTT | SEQ ID NO: 847 | CTCAGGCCCTCCTCTCCCCTC | SEQ ID NO: 3213 | CACTCCTTATTTGTGACACTAGC | SEQ ID NO: 5579 |
| rs2523619 | CAACTGCAGGGGTGACTGGA | SEQ ID NO: 848 | CAACTGCAGGGGTGACTGGG | SEQ ID NO: 3214 | TGTTGTTGTCCTCTTCCAG | SEQ ID NO: 5580 |
| rs3213483 | GGAAGGATCAGCGGGAGCTCTT | SEQ ID NO: 849 | GGAAGGATCAGCGGGAGCTCTG | SEQ ID NO: 3215 | CACAGTAAAAGGAAACCAGTT | SEQ ID NO: 5581 |
| rs2857713 | GGAGGTGTAGGGTGGTGCCACA | SEQ ID NO: 850 | GGAGGTGTAGGGTGGTGCCACG | SEQ ID NO: 3216 | ACCCTTGGGAGGAAGAGAC | SEQ ID NO: 5582 |
| rs2072632 | AGGAATGGCCTGTGGGGGAA | SEQ ID NO: 851 | AGGAATGGCCTGTGGGGGAG | SEQ ID NO: 3217 | GGATGGGAAAGGAAGAATC | SEQ ID NO: 5583 |
| rs2074469 | GAGAGGGTGCGCAGGAAGAA | SEQ ID NO: 852 | GAGAGGGTGCGCAGGAAGAG | SEQ ID NO: 3218 | TACATAGGGGACTGGAGGG | SEQ ID NO: 5584 |
| rs2071876 | ACGCCTTAGCGCTTCCAGCTCT | SEQ ID NO: 853 | ACGCCTTAGCGCTTCCAGGTCC | SEQ ID NO: 3219 | GCTCAGATTCCAGCTCCTC | SEQ ID NO: 5585 |
| rs3819686 | AGAATTTACGCCTGGACTCCTCAT | SEQ ID NO: 854 | AGAATTTACGCCTGGACTCCTCAG | SEQ ID NO: 3220 | AATTCTCTGTCTCTTCAGGGTC | SEQ ID NO: 5586 |
| rs1049414 | CAGCTCTCCTAGAGGGGGGCA | SEQ ID NO: 855 | CAGCTCTCCTAGAGGGGGGCG | SEQ ID NO: 3221 | GTGAGATGAAGAAGTGTCACAG | SEQ ID NO: 5587 |
| rs1048412 | TGACCTTTTTCCTGGGCAACTCTA | SEQ ID NO: 856 | TGACCTTTTTCCTGGGCAACTCTG | SEQ ID NO: 3222 | GGTGAAGGATACTGGGTTTTTA | SEQ ID NO: 5588 |
| rs2269420 | GGACACAGGGGCCTTGAAAGT | SEQ ID NO: 857 | GGACACAGGGGCCTTGAAAGG | SEQ ID NO: 3223 | AGGAACAGAGACACACAGAGAG | SEQ ID NO: 5589 |
| rs7750641 | CTGACCTTTGGCCCTGAAGGGT | SEQ ID NO: 858 | CTGACCTTTGGCCCTGAAGGGC | SEQ ID NO: 3224 | GTTCTACTTCATGTTCCAACAAG | SEQ ID NO: 5590 |
| rs2647027 | CCCTAGTCACTGCCAAGAGGAGCT | SEQ ID NO: 859 | CCCTAGTCACTGCCAAGAGGAGCC | SEQ ID NO: 3225 | AAAACACATTGATGTAGATATGTCC | SEQ ID NO: 5591 |
| rs3815095 | CCCTCCCCGCACAGGAAAA | SEQ ID NO: 860 | GCCTCGCCGCACAGGAAAT | SEQ ID NO: 3226 | CAACTCAGAGGACATCCCCTA | SEQ ID NO: 5592 |
| rs3130999 | GGCACTCAGCTGATGCCCTTT | SEQ ID NO: 861 | GGCACTCAGCTGATGCCCTTC | SEQ ID NO: 3227 | ATTAGCTTTAGGTCTCCACCAC | SEQ ID NO: 5593 |
| rs3134940 | CGCGCCCAGCCGTCTGTT | SEQ ID NO: 862 | CGCGCCCAGCCGTCTGTC | SEQ ID NO: 3228 | CTTTTTTTAGCTCAGAGGGAAG | SEQ ID NO: 5594 |
| rs743400 | CACCCCGGGCTATTTGACAGT | SEQ ID NO: 863 | CACCCCGGGCTATTTGACAGC | SEQ ID NO: 3229 | GAAATGTTAGCTTCTGAGGGAT | SEQ ID NO: 5595 |
| rs4711187 | TCTATTAGCTCCAGAAATACTTCCACAA | SEQ ID NO: 864 | TCTATTAGCTCCAGAAATACTTCCACAG | SEQ ID NO: 3230 | TATGATTTCATTGTTTTCACTTAGT | SEQ ID NO: 5596 |
| rs2395038 | CCTGGGTGACAGGAGGAGAA | SEQ ID NO: 865 | CCTGGGTGACAGGAGGAGAC | SEQ ID NO: 3231 | CTGTCCCCAAAACAAAAATAA | SEQ ID NO: 5597 |
| rs2239689 | AGGCCCACCAGGCATGCCT | SEQ ID NO: 866 | AGGCCCACCAGGCATGCCC | SEQ ID NO: 3232 | CCCATTCTATTTTCTGATGCA | SEQ ID NO: 5598 |
| rs995185 | GACAGTCCTGGCCCTGGCTA | SEQ ID NO: 867 | GACAGTCCTGGCCCTGGCTT | SEQ ID NO: 3233 | CATAGGAAGGGAAGAGTAAAGAG | SEQ ID NO: 5599 |
| rs3830041 | CAGCCATAGGGAAGAGGGGAAA | SEQ ID NO: 868 | CAGCCATAGGGAAGAGGGGAAG | SEQ ID NO: 3234 | GGGAACACACTGACCACCT | SEQ ID NO: 5600 |
| rs2523945 | CCCTCTCTGCCTTGGGACCTTA | SEQ ID NO: 869 | CCCTCTCTGCCTTGGGACCTTG | SEQ ID NO: 3235 | GCTGTTCCCACCTCTTGAC | SEQ ID NO: 5601 |
| rs3130099 | CCCAGCAAACACACGGGTTA | SEQ ID NO: 870 | CCCAGCAAACACACGGGTTG | SEQ ID NO: 3236 | AGCCAAGTAAATGACCAATAAAT | SEQ ID NO: 5602 |
| rs3130374 | AATATTGCCCAGTCGCACAGCTA | SEQ ID NO: 871 | AATATTGCCCAGTCGCACAGCTG | SEQ ID NO: 3237 | ACTCAGCCTGTCCCAGTTATAG | SEQ ID NO: 5603 |
| rs2524272 | GCCCAGAAACGTCAGGAAGGAAA | SEQ ID NO: 872 | GCCCAGAAACCTCAGGAAGGAAG | SEQ ID NO: 3238 | TTTGAACCATGCTCCCAGATA | SEQ ID NO: 5604 |
| rs3093558 | TTCTCCTTCAGCCTCCTCCCTTT | SEQ ID NO: 873 | TTCTCCTTCAGCCTCCTCCCTTC | SEQ ID NO: 3239 | CTTGAATTAAGCTCCTTGGAG | SEQ ID NO: 5605 |
| rs7656 | CTGGGAATTCTGGGCAGGCAT | SEQ ID NO: 874 | CTGGGAATTCTGGGCAGGCAC | SEQ ID NO: 3240 | AGCAGGCATCACAGAAGAGT | SEQ ID NO: 5606 |
| rs3093992 | CGTTTCCTGCCGCTATAACAGAATAA | SEQ ID NO: 875 | CGTTTCCTGCCGCTATAACAGAATAC | SEQ ID NO: 3241 | TGGGTAATTTATTTTGTTGTTTT | SEQ ID NO: 5607 |
| rs2428509 | CTGGOACGTTGAGGTCCCCTT | SEQ ID NO: 876 | CTGGCACGTTGAGGTCCCCTC | SEQ ID NO: 3242 | ATTACCCCATTCTACTGTGTCTT | SEQ ID NO: 5608 |
| rs2071292 | GCTCCTCCTCACCAGCCCACT | SEQ ID NO: 877 | GCTCCTCCTCACCAGCCCACC | SEQ ID NO: 3243 | CTACAGGCATGTAATGAGTTCAC | SEQ ID NO: 5609 |
| rs107822 | CACGGGAGGAAAGGGGGAATA | SEQ ID NO: 878 | CACGGGAGGAPAGGGGGAATG | SEQ ID NO: 3244 | GAAACAGGGACATACACAAGAT | SEQ ID NO: 5610 |
| rs3734860 | GAAATGGGGCTAATCATTTGCTTTAA | SEQ ID NO: 879 | GAAATGGGGCTAATCATTTGCAAAT | SEQ ID NO: 3245 | TACTAGGAGAGAAGGAAGGGATA | SEQ ID NO: 5611 |
| rs4151667 | GAGCAATCTCAGCCCCCAACA | SEQ ID NO: 880 | GAGCAATCTCAGCCCCCAACT | SEQ ID NO: 3246 | TGCCTGATGCCCTTTATCT | SEQ ID NO: 5612 |
| rs887466 | GTCCCACCCCAGCTTTCAGGTA | SEQ ID NO: 881 | GTCCCACCGCAGCTTTCAGGTG | SEQ ID NO: 3247 | CTCTAGGTCAGGAAGGATTGTT | SEQ ID NO: 5613 |
| rs2857096 | GGGCCCCTTCCACAATCCTG | SEQ ID NO: 882 | GGGCCCCTTCCACAATCCTC | SEQ ID NO: 3248 | TAAAATCCAAGAGTTCTCAGAAA | SEQ ID NO: 5614 |
| rs3130976 | CTGCAGATCAGGGGCCAGAT | SEQ ID NO: 883 | CTGCAGATCAGGGGCCAGAC | SEQ ID NO: 3249 | ATCCCTTTCTGGTCAATGTAATA | SEQ ID NO: 5615 |
| rs6910473 | CTGTTCCGAAGCATGTTCCTCCT | SEQ ID NO: 884 | CTGTTCCGAAGCATGTTCCTCCC | SEQ ID NO: 3250 | CATAATCTACCTGCCTTTTGG | SEQ ID NO: 5616 |
| rs1633012 | GGGAGAGGAGGGCCTGAGGG | SEQ ID NO: 885 | GGGAGAGGAGGGCCTGAGGC | SEQ ID NO: 3251 | TGAGAGGAATGGAGGGAAG | SEQ ID NO: 5617 |
| rs2523399 | CAAGCATGTTTGTACATAAAGATTCTCCTA | SEQ ID NO: 886 | CAAGCATGTTTGTACATAAAGATTCTCCTG | SEQ ID NO: 3252 | GGTTGGGATTTTAAAAATTCA | SEQ ID NO: 5618 |
| rs2071786 | AGGGGGCAAGGCGTCTGTTTT | SEQ ID NO: 887 | AGGGGGCAAGGCGTCTGTTTC | SEQ ID NO: 3253 | CAAAATGAGTAAGACAACAAAGG | SEQ ID NO: 5619 |
| rs3823359 | CACCTCTTGACCAGCTGCCTTC | SEQ ID NO: 888 | CACCTCTTGACCAGCTGCCTTG | SEQ ID NO: 3254 | GCCCTCAGCAAGTTCTCAC | SEQ ID NO: 5620 |
| rs400488 | GGACCAGGAGCTGGTGCTTCT | SEQ ID NO: 889 | GGACCAGGAGCTGGTGCTTCC | SEQ ID NO: 3255 | ATTGCTGTGGGGACTATGG | SEQ ID NO: 5621 |
| rs2239697 | AGCCGGCTGGATCTCTTGGAA | SEQ ID NO: 690 | AGCCGGCTGGATCTCTTGGAG | SEQ ID NO: 3256 | ATGGGGTCTGTAGACACAACTA | SEQ ID NO: 5622 |
| rs4959041 | GCCACATATGGAATTACAAATTTTTCTA | SEQ ID NO: 891 | GCCACATATGGAATTACAAATTTTTCTG | SEQ ID NO: 3257 | TTAGTCACATTAAAATGATAAAAGTG | SEQ ID NO: 5623 |
| rs1042434 | GCCTGGTGCTGGGCCTAGTC | SEQ ID NO: 892 | GCCTGGTGCTGGGCCTAGTG | SEQ ID NO: 3258 | GTCCTCATCATAAAGTCTCTGC | SEQ ID NO: 5624 |
| rs3419275 | GCTGAGTGCCGGCTCACCA | SEQ ID NO: 893 | GCTGAGTGCCGGCTCACCT | SEQ ID NO: 3259 | CCTGCTGAGTTCCACTGAC | SEQ ID NO: 5625 |
| rs381218 | AATTTCTGTGATTGGCTGAAGACAGA | SEQ ID NO: 894 | AATTTCTGTGATTGGCTGAAGACAGC | SEQ ID NO: 3260 | CTCGAATCTGTTTTCTTCTTCTT | SEQ ID NO: 5626 |
| rs29230 | CCTCACCTGGCAGACGAAAGGA | SEQ ID NO: 895 | CCTCACCTGGCAGACGAAAGGG | SEQ ID NO: 3261 | ACTGGTTCCTCCCAATGTG | SEQ ID NO: 5627 |
| rs2301226 | CCCAAGTGGCTAATCAGTCCTAATTATAA | SEQ ID NO: 896 | CCCAAGTGGCTAATCAGTCCTAATTATAG | SEQ ID NO: 3262 | CCCAATTATTAGTAATTAAAGAAATG | SEQ ID NO: 5628 |
| rs742870 | CTGTCAAGGCATCAGGGAAGGT | SEQ ID NO: 697 | CTGTCAAGGCATCAGGGAAGGC | SEQ ID NO: 3263 | GAACAGATGGCTTCAATGTAG | SEQ ID NO: 5629 |
| rs6905862 | AAGATGAAGGTCTGGACCACGACT | SEQ ID NO: 898 | AAGATGAAGGTCTGGACCACGACC | SEQ ID NO: 3264 | ATCCAATCTGATGGGAGTATG | SEQ ID NO: 5630 |
| rs3129938 | GAAACACAACTGTAAACAAGACAGATTTCT | SEQ ID NO: 699 | GAAACACAACTGTAAACAAGACAGATTTCA | SEQ ID NO: 3265 | TCCTGACTTGTGAAAATTCTATAA | SEQ ID NO: 5631 |
| rs1736939 | CTGGTGGGCACCATGCATTA | SEQ ID NO: 900 | CTGGTGGGCACCATGCATTG | SEQ ID NO: 3266 | GTTCTCTACAGTGACAGCCAGT | SEQ ID NO: 5632 |
| rs4548006 | CTGGGCTTTAAGGGACCCTCAT | SEQ ID NO: 901 | CTGGGCTTTAAGGGACCCTCAC | SEQ ID NO: 3267 | GAACCATTGTGACTGGAAATT | SEQ ID NO: 5633 |
| rs2857767 | CCTAGCATTTCCCTATGAGCTAACTATGG | SEQ ID NO: 902 | CCTAGCATTTCCCTATGAGCTAACTATGC | SEQ ID NO: 3268 | ATATAGTAACCCCATTTTACAGAGG | SEQ ID NO: 5634 |
| rs241442 | ACTGTCCCCTGCCCTCTCACA | SEQ ID NO: 903 | ACTGTCCCCTGCCCTCTCACG | SEQ ID NO: 3269 | TGCACATCTAGGGCACTAGTAG | SEQ ID NO: 5635 |
| rs3093671 | TTTGTGGTGAAGGAGGGGAGAGA | SEQ ID NO: 904 | TTTGTGGTGAAGGAGGGGAGAGG | SEQ ID NO: 3270 | GGTAATCCGGAAAGAGAAAAG | SEQ ID NO: 5636 |
| rs2857433 | GGGCATAAGTTGGATGACCTATGAA | SEQ ID NO: 905 | GGGCATAAGTTGGATGACCTATGAC | SEQ ID NO: 3271 | TTTAGTCTAAAGAATTGAGACTACCG | SEQ ID NO: 5637 |
| rs1800453 | AGCTGGCTCCCAGCCTCGT | SEQ ID NO: 906 | AGCTGGCTCCCAGCCTCGC | SEQ ID NO: 3272 | TACCTCTGCAGAGCAAAGG | SEQ ID NO: 5638 |
| rs1049628 | CTGCCCCTCCCTCAGGGAGT | SEQ ID NO: 907 | CTGCCCCTCCCTCAGGGAGC | SEQ ID NO: 3273 | AGTGACACTAAAACAAGAGGACA | SEQ ID NO: 5639 |
| rs2769 | GACCCCTGTTCCCATGCTGACT | SEQ ID NO: 908 | GACCCCTGTTCCCATGCTGACC | SEQ ID NO: 3274 | GTGTTTCCTCCCCAGTCAT | SEQ ID NO: 5640 |
| rs3763295 | CCACCTCCTCAAACTCACGGCA | SEQ ID NO: 909 | CCACCTCCTCAAACTCACGGCG | SEQ ID NO: 3275 | GAGAGCTGGAACTCTTACCAG | SEQ ID NO: 5641 |
| rs3763338 | TCCTAACCTATATTATTGCACCAGTGTA | SEQ ID NO: 910 | TCCTAACCTATATTATTGCACCAGTGTG | SEQ ID NO: 3276 | CAGTTTACAAGTATTTTTAAAGAA | SEQ ID NO: 5642 |
| rs6910733 | GTGATTGGATTGTGGCTGCCCA | SEQ ID NO: 911 | GTGATTGGATTGTGGCTGCCCG | SEQ ID NO: 3277 | AGCGAGAGAGGATTTTCCC | SEQ ID NO: 5643 |
| rs3830037 | GGTCCCCTCACCTCACCCAT | SEQ ID NO: 912 | GGTCCCCTCACCTCACCCAC | SEQ ID NO: 3278 | GTCTGACCACTGAGACACATAG | SEQ ID NO: 5644 |
| rs2858330 | AAATAAGTCAGCCACTGCACCTGGA | SEQ ID NO: 913 | AAATAAGTCAGCCACTGCACCTGGG | SEQ ID NO: 3279 | AACTATTCTGCTCAAAAAAAAAA | SEQ ID NO: 5645 |
| rs1078711 | GAGCAAAGAGTCAACAAAACCTTCTGT | SEQ ID NO: 914 | GAGCAAAGAGTCAACAAAACCTTCTGG | SEQ ID NO: 3280 | TGTTTTTTGAGATAAGGTCTCAC | SEQ ID NO: 5646 |
| rs2272874 | CGGCCTGTTGGCAGAAGAGTA | SEQ ID NO: 915 | CGGCCTGTTGGCAGAAGAGTG | SEQ ID NO: 3281 | CAGCTGACCTGATGAATATCA | SEQ ID NO: 5647 |
| rs762324 | AATCCCGTTTCCAGGCTAAATTGT | SEQ ID NO: 916 | AATCCCGTTTCCAGGCTAAATTGC | SEQ ID NO: 3282 | ATGACAGTTGACGATCTCCAT | SEQ ID NO: 5648 |
| rs2061952 | GGCGGAATAGTATTCCATCATGTATATATA | SEQ ID NO: 917 | GGCGGAATAGTATTCCATCATGTATATATG | SEQ ID NO: 3283 | CACATACTAATTACCCTGATTTGA | SEQ ID NO: 5649 |
| rs4647195 | CTATGCCTCCCCCTGCCAT | SEQ ID NO: 918 | CTATGCCTCCCCCTGCCAC | SEQ ID NO: 3284 | CCCCAGGAACTCAGTTGTT | SEQ ID NO: 5650 |
| rs2523400 | ATGCCACTGCCCTCCAGACCA | SEQ ID NO: 919 | ATGCCACTGCCCTCCAGACCG | SEQ ID NO: 3285 | GCTACAGAGTGAGAATTTGTCTC | SEQ ID NO: 5651 |
| rs1041981 | AGATGCATCTTGCCCACAGCAA | SEQ ID NO: 920 | AGATGCATCTTGCCCACAGCAC | SEQ ID NO: 3286 | ACCTCATTGGTAAACATCCAC | SEQ ID NO: 5652 |
| rs1367729 | TTTTCATTGCCTCAAAATTTGATTTTA | SEQ ID NO: 921 | TTTTCATTGCCTCAAAATTTGATTTTG | SEQ ID NO: 3287 | CTTCTCTATTTTTATATTTCATTGAA | SEQ ID NO: 5653 |
| rs2523943 | GCTCCCATCCCCAGCTCATTA | SEQ ID NO: 922 | GCTCCCATCCCCAGCTCATTC | SEQ ID NO: 3288 | GGTTCTTTCGTCTCTGTCAAC | SEQ ID NO: 5654 |
| rs1233421 | CAGAAAGGCCAAGGTTCCCACA | SEQ ID NO: 923 | CAGAAAGGCCAAGGTTCCCACC | SEQ ID NO: 3289 | AACTGTGAGCCACACTGCAC | SEQ ID NO: 5655 |
| rs4988888 | CCCTGCTGGTAAATGATTCTTCATTAT | SEQ ID NO: 924 | CCCTGCTGGTAAATGATTCTTCATTAC | SEQ ID NO: 3290 | CTCTTCGAGTGAAAGTATTTGAA | SEQ ID NO: 5656 |
| rs4713260 | CACTGTTCCCTCCTCAGGGACTA | SEQ ID NO: 925 | CACTGTTCCCTCCTCAGGGACTG | SEQ ID NO: 3291 | AATTTTCCACGGAATTCG | SEQ ID NO: 5657 |
| rs3095227 | ACAGGCCCCTTCTGCGGAGA | SEQ ID NO: 926 | ACAGGCCCCTtCTGCGGAGG | SEQ ID NO: 3292 | CAGAATGCTCATCTGATTAAGAC | SEQ ID NO: 5658 |
| rs2596538 | AGGTGGGGTGGAGGGGGAGA | SEQ ID NO: 927 | AGGTGGGGTGGAGGGGGAGG | SEQ ID NO: 3293 | CTTTGGACAGAAGACCTGG | SEQ ID NO: 5659 |
| rs1480382 | ATCAAAGCAGTCATGTGTGCAATT | SEQ ID NO: 928 | ATCAAAGCAGTCATGTGTGCAATC | SEQ ID NO: 3294 | ACAGAAAAGAATAGAAATTTACTTAAG | SEQ ID NO: 5660 |
| rs2229699 | GGGACGCAGTTCTCGGACGA | SEQ ID NO: 929 | GGGACGCAGTTCTCGGACGC | SEQ ID NO: 3295 | GACGGCCTCTATTACCTCTACT | SEQ ID NO: 5661 |
| rs2276447 | GGCTCCTGCTTTCCCTGAGAAC | SEQ ID NO: 930 | GGCTCCTGCTTTCCCTGAGAAG | SEQ ID NO: 3296 | GATATGACCCCTCATCCCC | SEQ ID NO: 5662 |
| rs2074511 | CTCAGCCTCTGACCGCCCA | SEQ ID NO: 931 | CTCAGCCTCTGACCGCCCG | SEQ ID NO: 3297 | CCACCCAACAGTCCTCTTCT | SEQ ID NO: 5663 |
| rs2285795 | GAGGAAGGAGGCAGTGAGTGGT | SEQ ID NO: 932 | GAGGAAGGAGGCAGTGAGTGGC | SEQ ID NO: 3298 | ACATCTGCCCATAATGACAAT | SEQ ID NO: 5664 |
| rs2523959 | CATCACTCTTGCCTGCTGCCAA | SEQ ID NO: 933 | CATCACTCTTGCCTGCTGCCAC | SEQ ID NO: 3299 | TCTCACCTCCCTTCTGTCC | SEQ ID NO: 5665 |
| rs5020983 | CATCACTCTTGCCTGCTGCCAA | SEQ ID NO: 934 | CATCACTCTTGCCTGCTGCCAC | SEQ ID NO: 3300 | TCTCACCTCCCTTCTGTCC | SEQ ID NO: 5666 |
| rs2071353 | CCTCCAGGCTGGATGCAGACA | SEQ ID NO: 935 | CCTCCAGGCTGGATGCAGACG | SEQ ID NO: 3301 | ACTTGCCAACTTCTCTCTCTG | SEQ ID NO: 5667 |
| rs1610646 | GCTTGCTTACGATGGCAAGGATTA | SEQ ID NO: 936 | GCTTGCTTACGATGGCAAGGATTT | SEQ ID NO: 3302 | ATCGTCCTGAACGAGGAC | SEQ ID NO: 5668 |
| rs3117035 | GCTGAAAAGGGGCGTCGTGTT | SEQ ID NO: 937 | GCTGAAAAGGGGCGTCGTGTC | SEQ ID NO: 3303 | CTCCTCCTGAGGTTGATCTAAG | SEQ ID NO: 5669 |
| rs2071470 | CATTCTGGAGAAAGGAAAAAAATGAGAT | SEQ ID NO: 938 | CATTCTGGAGAAAGGAAAAAAATGAGAC | SEQ ID NO: 3304 | GTAAAATCGTCAGCCTCTTCA | SEQ ID NO: 5670 |
| rs376892 | CCCTTTCCCATGACCCCCA | SEQ ID NO: 939 | CCCTTTCCCATGACCCCCG | SEQ ID NO: 3305 | TGGCACCTGATTACGTCACT | SEQ ID NO: 5671 |
| rs1631950 | CCCCACAGGCGGTGTATGGA | SEQ ID NO: 940 | CCCCACAGGCGGTGTATGGG | SEQ ID NO: 3306 | GATTCTCTCCTCCTTCTCCTAAC | SEQ ID NO: 5672 |
| rs206781 | TCCTCCAGAAGGTCCAAAGCCA | SEQ ID NO: 941 | TCCTCCAGAAGGTCCAAAGCCG | SEQ ID NO: 3307 | AAGGGCCTAAAGCAGCACT | SEQ ID NO: 5673 |
| rs2071592 | ATGGCGCAAGCAGAGACGCT | SEQ ID NO: 942 | ATGGCGCAAGCAGAGACGCA | SEQ ID NO: 3308 | GTGGAGGACGGAAGTGAACT | SEQ ID NO: 5674 |
| rs4713274 | ACCCTTTTTGGTCTTATTTTCACTTAG | SEQ ID NO: 943 | ACCCTTTTTGGTCTTATTTTCACTTAC | SEQ ID NO: 3309 | TATCTTTGTTTATTAACTGTTCTGG | SEQ ID NO: 5675 |
| rs4959119 | CCCACCTATAGTACTGCTGCTACTCAAT | SEQ ID NO: 944 | CCCACCTATAGTACTGCTGCTACTCAAC | SEQ ID NO: 3310 | ATTTTCTTCATGTATTAGAAGAATT | SEQ ID NO: 5676 |
| rs3129744 | CAAACCAAAACAAGTCAAATTACCATAAA | SEQ ID NO: 945 | CAAACCAAAACAAGTCAAATTACCATAAC | SEQ ID NO: 3311 | GACAAAAATCTTTACATTAAAAGAG | SEQ ID NO: 5677 |
| rs1737086 | TTTCTTGGATATAACACCAAAGATACAAA | SEQ ID NO: 946 | TTTCTTGGATATAACACCAAAGATACAAC | SEQ ID NO: 3312 | ACAAAGAAAAAATTGATAAATTGG | SEQ ID NO: 5678 |
| rs2523960 | AGCAGGCAAGAGTGATGGGAGA | SEQ ID NO: 947 | AGCAGGCAAGAGTGATGGGAGG | SEQ ID NO: 3313 | CTTTTCCAGGAGAGCTGG | SEQ ID NO: 5679 |
| rs5020984 | AGCAGGCAAGAGTGATGGGAGA | SEQ ID NO: 948 | AGCAGGCAAGAGTGATGGGAGG | SEQ ID NO: 3314 | CTTTTCCAGGAGAGCTGG | SEQ ID NO: 5680 |
| rs3093542 | AGGGAGCCCCTAGGGGAGAAC | SEQ ID NO: 949 | AGGGAGCCCCTAGGGGAGAAG | SEQ ID NO: 3315 | GAGTTGAGGGGGGCTCTAG | SEQ ID NO: 5681 |
| rs2127680 | AGGAGTGGGAGCAGGGTCATA | SEQ ID NO: 950 | AGGAGTGGGAGCAGGGTCATG | SEQ ID NO: 3316 | CATCTTAAGGACAAGGGAATG | SEQ ID NO: 5682 |
| rs2844508 | CTGCTGTGGATATCCCCAGCACG | SEQ ID NO: 951 | CTGCTGTGGATATCCCCAGCACC | SEQ ID NO: 3317 | ACCTGAGAAGATGCCCTG | SEQ ID NO: 5683 |
| rs3093554 | TGGACTCTTTGCCCCTTCCAGA | SEQ ID NO: 952 | TGGACTCTTTGCCCCTTCCAGG | SEQ ID NO: 3318 | TTTCAGAAGCTGCCAGAG | SEQ ID NO: 5684 |
| rs1268928 | AGGGGCCCCTAGAGGGCCT | SEQ ID NO: 953 | AGGGGCCCCTAGAGGGCCG | SEQ ID NO: 3319 | GGGGATGGCTAGAAAACAG | SEQ ID NO: 5685 |
| rs2294881 | TCTCTATGGAAGTCCTAAGACTGAAATCA | SEQ ID NO: 954 | TCTCTATGGAAGTCCTAAGACTGAAATCG | SEQ ID NO: 3320 | TGCTTGTAGTCTTACAAATAATGTT | SEQ ID NO: 5686 |
| rs7773149 | GGGACACAAAACCAGTTAAAGATAGAAAGA | SEQ ID NO: 955 | GGGACACAAAACCAGTTAAAGATAGAAAGG | SEQ ID NO: 3321 | CATCTATTATCTITTAGTCAATGACC | SEQ ID NO: 5687 |
| rs2524197 | GGAAGCGAAAGGGGAGCTGAGT | SEQ ID NO: 956 | GGAAGCGAAAGGGGAGCTGAGC | SEQ ID NO: 3322 | TCTGTCCTCAGTCCTTGGG | SEQ ID NO: 5688 |
| rs1419635 | CGGAATACAGCTCCTAGGTTTTTGTGT | SEQ ID NO: 957 | CGGAATACAGCTCCTAGGTTTTTGTGC | SEQ ID NO: 3323 | AATTCACCAATCTTATAATTAAATATT | SEQ ID NO: 5689 |
| rs7755774 | GTCCAGGCCCCCTTCTAGTTTCC | SEQ ID NO: 958 | GTCCAGGCCCCCTTCTAGTTTCG | SEQ ID NO: 3324 | GACATGGGGATGCTGTTT | SEQ ID NO: 5690 |
| rs3131001 | TGTGTTTGCTGTGCCAGCCTTT | SEQ ID NO: 959 | TGTGTTTGCTGTGCCAGCCTTG | SEQ ID NO: 3325 | CTCTGCTGTGTTTTGGAAGTT | SEQ ID NO: 5691 |
| rs1610676 | TTGTGTTTTCTATTCATATTGCTATTCCA | SEQ ID NO: 960 | TTGTGTTTTCTATTCATATTGCTATTCCG | SEQ ID NO: 3326 | CACTGGTTATTCCTTTCTACTCTT | SEQ ID NO: 5692 |
| rs6901686 | GGACGGAGGGAAGACCCTGA | SEQ ID NO: 961 | GGACGGAGGGAAGACCCTGG | SEQ ID NO: 3327 | AGAGTTAGGGTCAGGGAGAGTT | SEQ ID NO: 5693 |
| rs3094200 | GGACTCACCTCTGCAGCCACAA | SEQ ID NO: 962 | GGACTCACCTCTGCAGCCACAG | SEQ ID NO: 3328 | AACACCCTCCTCTCCTTG | SEQ ID NO: 5694 |
| rs2523705 | TGTTGCTGCTAGTTATTATGAGGTAGTTT | SEQ ID NO: 963 | TGTTGCTGCTAGTTATTATGAGGTAGTTC | SEQ ID NO: 3329 | AGCTGGAAGAATAGAGAATTAAAA | SEQ ID NO: 5695 |
| rs885948 | CTGCTGGGGTCAGTGGCTCTA | SEQ ID NO: 964 | CTGCTGGGGTCAGTGGCTCTG | SEQ ID NO: 3330 | CTGCCTTCTAAGGGGATG | SEQ ID NO: 5696 |
| rs2009658 | CCTCTAACACTCTCCAAGTAAAATCACATC | SEQ ID NO: 965 | CCTCTAACACTCTCCAAGTAAAATCACATG | SEQ ID NO: 3331 | GTAGCAGTAATAATATTTGAGGTGA | SEQ ID NO: 5697 |
| rs2517754 | GTTTCCTGAAAGTGTAACTGCTGATCT | SEQ ID NO: 966 | GTTTCCTGAAAGTGTAACTGCTGATCC | SEQ ID NO: 3332 | GGACAGGATCAGGAAACAT | SEQ ID NO: 5698 |
| rs2596499 | ACAGAGGCTCACACATCGCCCA | SEQ ID NO: 967 | ACAGAGGCTCACACATCGCCCT | SEQ ID NO: 3333 | GTCTCTGTGCATTCTGAGACA | SEQ ID NO: 5699 |
| rs929156 | AGGGGGCTGTCTGGCAGGT | SEQ ID NO: 968 | AGGGGGCTGTCTGGCAGGC | SEQ ID NO: 3334 | GTACCTCACTGACTTCCTGTCTT | SEQ ID NO: 5700 |
| rs2076312 | TGCTGCTCTCTGGGCCTCTTTA | SEQ ID NO: 969 | TGCTGCTCTCTGGGCCTCTTTC | SEQ ID NO: 3335 | TAAAATGGGGGTCAGCTAAAT | SEQ ID NO: 5701 |
| rs3093666 | GAGACACGGAGCCCAGCCT | SEQ ID NO: 970 | GAGACACGGAGCCCAGCCC | SEQ ID NO: 3336 | CCAGCTCCCTCTATTTATGTTT | SEQ ID NO: 5702 |
| rs3132520 | GGGCACCAAGGGCCCCCT | SEQ ID NO: 971 | GGGCACCAAGGGCCCCCC | SEQ ID NO: 3337 | CAGGGACCTAGGTATCTAGTTCC | SEQ ID NO: 5703 |
| rs7773175 | TTGTGTGACTGCCTTAAAACTAATGGG | SEQ ID NO: 972 | TTGTGTGACTGGCTAAAACTAATGGC | SEQ ID NO: 3338 | AAAGGGGTTACAAAAATATCAAG | SEQ ID NO: 5704 |
| rs3116956 | CTAGCCGGGCTGGAGTTGAGA | SEQ ID NO: 973 | CTAGCCGGGCTGGAGTTGAGG | SEQ ID NO: 3339 | TTCTTTGGCGCTCTTTCT | SEQ ID NO: 5705 |
| rs3823417 | CTGCCCCTGGAAAAGGTCCCA | SEQ ID NO: 974 | CTGCCCCTGGAAAAGGTCCCG | SEQ ID NO: 3340 | GTTGGGAGTGATTTAGCACTCT | SEQ ID NO: 5706 |
| rs1265092 | GGAGCTCAGTGCGGGGA | SEQ ID NO: 975 | GGAGCTTTCAGTGCGGGGG | SEQ ID NO: 3341 | GTAGTAGCTGACACTGTCCCAG | SEQ ID NO: 5707 |
| rs204889 | GCTTGAGCCACCTCGCCT | SEQ ID NO: 976 | GCTTGAGCCACCTCGCCC | SEQ ID NO: 3342 | GCCTCCTGCTCCATTATTTT | SEQ ID NO: 5708 |
| rs910320 | GTTGGCGACATGATGCATGCA | SEQ ID NO: 977 | GTTGGCGACATGATGCATGCG | SEQ ID NO: 3343 | ATGCCCTCAAAGTCAGCT | SEQ ID NO: 5709 |
| rs2239886 | AGCTCAGCCCAGCCCCTCAA | SEQ ID NO: 978 | AGCTCAGCCCAGCCCCTCAC | SEQ ID NO: 3344 | TTAGAGAGAGCTCACAAAATGTC | SEQ ID NO: 5710 |
| rs2158279 | CATAGATGTCCAATGATAGGAGACTGATA | SEQ ID NO: 979 | CATAGATGTCCAATGATAGGAGACTGATC | SEQ ID NO: 3345 | ATCAAATATAATACATGAACAAAGTG | SEQ ID NO: 5711 |
| rs463260 | GCGATCCACGTGCAAAACTCT | SEQ ID NO: 980 | GCGATCCACGTGCAAAACTCC | SEQ ID NO: 3346 | CTCAGCTGCCACACAGTC | SEQ ID NO: 5712 |
| rs2523713 | CCCCCGTGCCCGGCTTTT | SEQ ID NO: 981 | CCCCCGTGCCCGGCTTTC | SEQ ID NO: 3347 | GCTAGTTTTTGTAGCTCATAGTGA | SEQ ID NO: 5713 |
| rs2072915 | CCAGTATCCACCTCTGGGGGAGT | SEQ ID NO: 982 | CCAGTATCCACCTCTGGGGGAGA | SEQ ID NO: 3348 | CCCCATTTCCACTCTTCAG | SEQ ID NO: 5714 |
| rs2187578 | CCACACTGTCAATGAAAAGAGTCAAACT | SEQ ID NO: 983 | CCACACTGTCAATGAAAAGAGTCAAACG | SEQ ID NO: 3349 | TAAAGAGATTTATTCTGAGCCAA | SEQ ID NO: 5715 |
| rs2246626 | CAGGGCAGATGTTCCCTCACAGA | SEQ ID NO: 984 | CAGGGCAGATGTTCCCTCACAGG | SEQ ID NO: 3350 | TCAGCCCCTGAATATTGG | SEQ ID NO: 5716 |
| rs2517672 | CAGCCATAATCACGAGAGATGTAAGAAT | SEQ ID NO: 985 | CAGCCATAATCACGAGAGATGTAAGAAC | SEQ ID NO: 3351 | ATTTTTCAAACCAACCTTCTAAC | SEQ ID NO: 5717 |
| rs2076489 | ATCGAGCCCCAGGGGGAA | SEQ ID NO: 986 | ATCGAGCCCCAGGGGGAG | SEQ ID NO: 3352 | ACAGCAGCTAAAGCCAGTG | SEQ ID NO: 5718 |
| rs1264322 | ATCTCCACCTGGGGGGGCGA | SEQ ID NO: 987 | ATCTCCACCTGGGGGGGCGG | SEQ ID NO: 3353 | ATCCAGTGGACCTTAGAGCAT | SEQ ID NO: 5719 |
| rs2523970 | GTGCCCAGTGTGTAACAGGCCA | SEQ ID NO: 988 | GTGGCCAGTGTGTAACAGGCCC | SEQ ID NO: 3354 | GTGCTTCAGAGTTGATGACC | SEQ ID NO: 5720 |
| rs2073722 | ACCACGCCTTCTGCTCCACCA | SEQ ID NO: 989 | ACCACGCCTTCTGCTCCACCC | SEQ ID NO: 3355 | GAAATCTGTTCACCGAGTGTT | SEQ ID NO: 5721 |
| rs6915224 | TGAATAGCTCTGTAAGGCACTAAAACAT | SEQ ID NO: 990 | TGAATAGCTCTGTAAGGCACTAAAACAC | SEQ ID NO: 3356 | CTATACATTTACTTTTAGAAATGCA | SEQ ID NO: 5722 |
| rs209130 | GGACTAAAAAGGAAAGAAGCTAACAATGGT | SEQ ID NO: 991 | GGACTAAAAAGGAAAGAAGCTAACAATGGC | SEQ ID NO: 3357 | GTCTATGAAAATGAAATAAACAAAA | SEQ ID NO: 5723 |
| rs5017092 | GGCTGCATGATAAAAATTAAATGATTA | SEQ ID NO: 992 | GGCTGCATGATTTTAAATTAAATGATTC | SEQ ID NO: 3358 | TAAAAATTGTTATCTGAAAATTTCA | SEQ ID NO: 5724 |
| rs3130552 | AGAGGCTCTGAGAGGTGGCCTA | SEQ ID NO: 993 | AGAGGCTCTGAGAGGTGGCCTG | SEQ ID NO: 3359 | CTACAGGGACAAGCACAATC | SEQ ID NO: 5725 |
| rs2247720 | CACCCCTTTCCTGGCCAA | SEQ ID NO: 994 | CACCCCTTTCCTGGCCAG | SEQ ID NO: 3360 | GCCCCAGTAACCTACAAGG | SEQ ID NO: 5726 |
| rs3807036 | AAAGTCCTGCTGCTCCATAAAGAT | SEQ ID NO: 995 | AAAGTCCTGCTGCTCCATAAAGAC | SEQ ID NO: 3361 | TACTGCTGTTATACTGTAATTCTAATT | SEQ ID NO: 5727 |
| rs1049622 | GGGCTGACCGGGTCATGAGT | SEQ ID NO: 996 | GGGCTGACCGGGTCATGAGC | SEQ ID NO: 3362 | TCTGTCTGCGGGTAGAGCT | SEQ ID NO: 5728 |
| rs2179915 | GCCTTTCGAATTAACCCAGTCAGAA | SEQ ID NO: 997 | GCCTTTCGAATTAACCCAGTCAGAC | SEQ ID NO: 3363 | TTAATAAATAAACAAAGCCTCCA | SEQ ID NO: 5729 |
| rs6930217 | CCTTTACGATAGGTCTTGAAGTTAGGTAT | SEQ ID NO: 998 | CCTTTACGATAGGTCTTGAAGTTAGGTAC | SEQ ID NO: 3364 | GTTAGCCATCTTACTTAATTCTTCT | SEQ ID NO: 5730 |
| rs3093553 | AGCTGGGAGCCCCTGAGGGT | SEQ ID NO: 999 | AGCTGGGAGCCCCTGAGGGG | SEQ ID NO: 3365 | AAGGAGAGACAGTCTGCTCTTAC | SEQ ID NO: 5731 |
| rs6457597 | GGGCTATGGGATGGGATAGATAGAT | SEQ ID NO: 1000 | GGGCTATGGGATGGGATAGATAGAC | SEQ ID NO: 3366 | AACCCTTTATTTCCTCGTTTCT | SEQ ID NO: 5732 |
| rs241437 | ACACAGGTATCTTCTACAAATTGTAAGCT | SEQ ID NO: 1001 | ACACAGGTATCTTCTACAAATTGTAAGCC | SEQ ID NO: 3367 | GCTCCTTCAGTAAAAAAGAGAAA | SEQ ID NO: 5733 |
| rs5026740 | CGGTGGCTCAAACCTATAACCCT | SEQ ID NO: 1002 | CGGTGGCTCAAACCTATAACCCA | SEQ ID NO: 3368 | GCATGTTGGGAGGTCAAG | SEQ ID NO: 5734 |
| rs15604 | ATCCGCGGGGAGACAGGCA | SEQ ID NO: 1003 | ATCCGCGGGGAGACAGGCG | SEQ ID NO: 3369 | TTAACGACGACTCACACGAT | SEQ ID NO: 5735 |
| rs2071800 | CCAAGGCCTGCGTTCAGTTGA | SEQ ID NO: 1004 | CCAAGGCCTGCGTTCAGTTGG | SEQ ID NO: 3370 | ACACCAAGGGCTCTTATGAAT | SEQ ID NO: 5736 |
| rs2844522 | ATGGTCTTGAGCTCCAGGGGAG | SEQ ID NO: 1005 | ATGGTCTTGAGCTCCAGGGGAC | SEQ ID NO: 3371 | TGTTTCTTAGTGCCCAGGC | SEQ ID NO: 5737 |
| rs2516409 | GGCCTCACAACCTAATTTTTAAGTTCA | SEQ ID NO: 1006 | GGCCTCACAACCTTTTTTTTTAAGTTCC | SEQ ID NO: 3372 | CTATCCCTGTCATTACTAACTATTCC | SEQ ID NO: 5738 |
| rs7770592 | TCAATCTACTACCTCTTTCGCATAATCAA | SEQ ID NO: 1007 | TCAATCTACTACCTCTTTCGCATAATCAC | SEQ ID NO: 3373 | TCTATCATTCTTATCTGTATAGTCAGC | SEQ ID NO: 5739 |
| rs444013 | GAGGGTGACCACCCTGGGG | SEQ ID NO: 1008 | GAGGGTGACCACCCTGGGC | SEQ ID NO: 3374 | GGTCAAAAAGCTTATTTCTTTTAA | SEQ ID NO: 5740 |
| rs2844846 | CTGTGCCCTCTGGTGAGCCA | SEQ ID NO: 1009 | CTGTGCCCTCTGGTGAGCCT | SEQ ID NO: 3375 | GGGCAACTCTCAAGGGTTT | SEQ ID NO: 5741 |
| rs3757346 | AAAGGGAGCCTGGGGCTGTC | SEQ ID NO: 1010 | AAAGGGAGCCTGGGGCTGTG | SEQ ID NO: 3376 | GGGTGAAGCTGGCCTATTT | SEQ ID NO: 5742 |
| rs3134929 | GAGAATTGCAAGGTGGCTGCCG | SEQ ID NO: 1011 | GAGAATTGCAAGGTGGCTGCCC | SEQ ID NO: 3377 | AGGACCTGGAAACAGGTATTG | SEQ ID NO: 5743 |
| rs401775 | GGAACGTGGGAAGGAACCCA | SEQ ID NO: 1012 | GGAACGTGGGAAGGAACCCG | SEQ ID NO: 3378 | CCTCCAACTTTACCCAGTCTT | SEQ ID NO: 5744 |
| rs6924267 | TGGGCAACAGAGACAGACTCCA | SEQ ID NO: 1013 | TGGGCAACAGAGACAGACTCCG | SEQ ID NO: 3379 | AAAAGAGTTGTTCTATGAACAGCT | SEQ ID NO: 5745 |
| rs2517673 | CTGGTTAGAAGGTTGGTTTGAAAAATA | SEQ ID NO: 1014 | CTGGTTAGAAGGTTGGTTTGAAAAATG | SEQ ID NO: 3380 | TCTTACATCTCTCGTGATTATGG | SEQ ID NO: 5746 |
| rs1736980 | GAATGAACTAATTCAGAAAATCGGTACCA | SEQ ID NO: 1015 | GAATGAACTAATTCAGAAAATCGGTACCG | SEQ ID NO: 3381 | GAGTTGGGTATTACTATAAAAATTCT | SEQ ID NO: 5747 |
| rs1611203 | AAGTCCCAGGGCCTCAGGCA | SEQ ID NO: 1016 | AAGTCCCAGGGCCTCAGGCC | SEQ ID NO: 3382 | GGCTCTCAGGATCTCAGG | SEQ ID NO: 5748 |
| rs7768484 | ACCTGCGGATCGCGCTCCT | SEQ ID NO: 1017 | ACCTGCGGATCGCGCTCCG | SEQ ID NO: 3383 | TACTACAACCAGAGCGAGGG | SEQ ID NO: 5749 |
| rs7741100 | CCCGATCAAACCCCCGGT | SEQ ID NO: 1018 | CCCGATCAATCCCCCGGG | SEQ ID NO: 3384 | ATTTGGGCCTGAACTGAAAAT | SEQ ID NO: 5750 |
| rs2294884 | AAAGTGAACAGGTTGAATTTAATCTCTA | SEQ ID NO: 1019 | AAAGTGAACAGGTTGAATTTAATCTCTC | SEQ ID NO: 3385 | TGTAGTGTCCAGTTCTAAGAGATT | SEQ ID NO: 5751 |
| rs2308935 | CCCACCTTTACAGTATTTCACAGGGT | SEQ ID NO: 1020 | CCCACCTTTACAGTATTTCACAGGGG | SEQ ID NO: 3386 | CCAGAACGCAGAGACTTTATG | SEQ ID NO: 5752 |
| rs2282850 | AGGCGGAGAGAGAGCCGGTT | SEQ ID NO: 1021 | AGGCGGAGAGAGAGCCGGTC | SEQ ID NO: 3387 | GATTACCCTCCCTACCACAAC | SEQ ID NO: 5753 |
| rs2073154 | GGTCAAATGCCATCACGGCG | SEQ ID NO: 1022 | GGTCAAATGCCATCACGGCC | SEQ ID NO: 3388 | ACAACATGGACTCCGTGCT | SEQ ID NO: 5754 |
| rs1265095 | GGGGACAGGAATTCAGGGGAT | SEQ ID NO: 1023 | GGGGACAGGAATTCAGGGGAC | SEQ ID NO: 3389 | CAGAAGGTGCATCTGGCT | SEQ ID NO: 5755 |
| rs3095317 | GGACCTCATTGGGGTTTGGGT | SEQ ID NO: 1024 | GGACCTCATTGGGGTTGGGC | SEQ ID NO: 3390 | CAAGTGTGGAAGAAAAAGCTG | SEQ ID NO: 5756 |
| rs3778630 | GGTCCAATGCACGGGCAT | SEQ ID NO: 1025 | GGTCCAATGCACGGGCAC | SEQ ID NO: 3391 | GAATCTGTTGCTGTAGAACACAG | SEQ ID NO: 5757 |
| rs3869090 | GGGTGATGCGGATGCTCTTTT | SEQ ID NO: 1026 | GGGTGATGCGGATGCTCTTTC | SEQ ID NO: 3392 | CTGCTGTAGCTCTGTGGGTAA | SEQ ID NO: 5758 |
| rs415929 | GTGGCACTGGATCTGGGGGTAT | SEQ ID NO: 1027 | GTGGCACTGGATCTGGGGGTAC | SEQ ID NO: 3393 | CTCCATTAACACATGGGTTG | SEQ ID NO: 5759 |
| rs2747431 | CTACCTTCCCCGATGCCCATCT | SEQ ID NO: 1028 | CTACCTTCCCCGATGCCCATCC | SEQ ID NO: 3394 | AGAATCTTCCTTCTGGGATG | SEQ ID NO: 5760 |
| rs2428507 | CCTCAGCCCTGCCCTGCT | SEQ ID NO: 1029 | CCTCAGCCCTGCCCTGCC | SEQ ID NO: 3395 | AAGGTCTCCTGCTTTCCCT | SEQ ID NO: 5761 |
| rs2295666 | CAACCACCGCACATCCACTGT | SEQ ID NO: 1030 | CAACCACCGCACATCCACTGC | SEQ ID NO: 3396 | CACCTGCTGTCACTGTCG | SEQ ID NO: 5762 |
| rs1233427 | CTGTGACTGGCCAGGGACCT | SEQ ID NO: 1031 | CTGTGACTGGCCAGGGACCC | SEQ ID NO: 3397 | ACACCCCAAGTCAGATGC | SEQ ID NO: 5763 |
| rs7774644 | ACCGCCAGTGCCTTGGGA | SEQ ID NO: 1032 | ACCGCCAGTGCCTTGGGG | SEQ ID NO: 3398 | GGGGATCACAGAAGGTGAC | SEQ ID NO: 5764 |
| rs3095309 | GGAGGGGTGGAGGGGACCA | SEQ ID NO: 1033 | GGAGGGGTGGAGGGGACCG | SEQ ID NO: 3399 | CTGCTTCATAATACATGGCTG | SEQ ID NO: 5765 |
| rs5872 | CCCACCTGTCCTGCAGTTTTTGT | SEQ ID NO: 1034 | CCCACCTGTCCTGCAGTCTTTGA | SEQ ID NO: 3400 | CCTTTCAGGAACCCTGTATG | SEQ ID NO: 5766 |
| rs1033500 | GAGTTGTATATATGGGAAACTTACAAGGAA | SEQ ID NO: 1035 | GAGTTGTATATATGGGPAACTTACAAGGAG | SEQ ID NO: 3401 | TTCTTCAGTTGTTTGTAAACATTT | SEQ ID NO: 5767 |
| rs3763306 | TGGTGTAAGGGAAAGGAGAGAAACTA | SEQ ID NO: 1036 | TGGTGTAAGGGAAAGGAGAGAAACTG | SEQ ID NO: 3402 | GCAATTTATACATTATATAGGGAAGC | SEQ ID NO: 5768 |
| rs635688 | TTTTTCTGTGGTTCTGACCTAACATTTT | SEQ ID NO: 1037 | TTTTTCTGTGGTTCTGACCTAACATTTC | SEQ ID NO: 3403 | TTATAAAACAGCCTATGGACATG | SEQ ID NO: 5769 |
| rs4151669 | CTTGTGCCAATGGGGATGCCT | SEQ ID NO: 1038 | CTTGTGCCAATGGGGATGCCC | SEQ ID NO: 3404 | CAGAGGTATGAGACATCGAGGTA | SEQ ID NO: 5770 |
| rs2076527 | TGGAGCTGTTCTTAGAGATGACATTA | SEQ ID NO: 1039 | TGGAGCTGTTCTTAGAGATGACATTG | SEQ ID NO: 3405 | CATTGTGTTTTGTCCACTCTATT | SEQ ID NO: 5771 |
| rs3178162 | CGGATCTGGTCGGTGATGGTT | SEQ ID NO: 1040 | CGGATCTGGTCGGTGATGGTG | SEQ ID NO: 3406 | AGGAGCAAGGGTTGACTGTAG | SEQ ID NO: 5772 |
| rs241449 | AGTCCATCAGCCGCTGCTGA | SEQ ID NO: 1041 | AGTCCATCAGCCGCTGCTGC | SEQ ID NO: 3407 | GAATAGAGGTCCTGTCCCTC | SEQ ID NO: 5773 |
| rs4435981 | CTGCTCCCTGCTGCAAAAAACA | SEQ ID NO: 1042 | CTGCTCCCTGCTGCAAAAAACG | SEQ ID NO: 3408 | TGGCAAGATCCTGATTGC | SEQ ID NO: 5774 |
| rs6456897 | TGGCATAGTAGAAAAGAAACAAGAATGATA | SEQ ID NO: 1043 | TGGCATAGTAGAAAAGAAACAAGAATGATG | SEQ ID NO: 3409 | TAGAAAATCTAGATTTAGTCCACTTC | SEQ ID NO: 5775 |
| rs3828923 | CATTCACACCCACCCACCAT | SEQ ID NO: 1044 | CATTCACACCCACCCACCAC | SEQ ID NO: 3410 | CTTGAAAGCCTCTGGAATCT | SEQ ID NO: 5776 |
| rs2179920 | AATGTGGTTAACACATTAGTGGACTCT | SEQ ID NO: 1045 | AATGTGGTTAACACATTAGTGGACTCC | SEQ ID NO: 3411 | CAAAATTCATATTTAATTTAAACTGAA | SEQ ID NO: 5777 |
| rs3218806 | GCTCACCACCAAGTCGCCAT | SEQ ID NO: 1046 | GCTCACCACCAAGTCGCCAG | SEQ ID NO: 3412 | CTCCAGGCACTTCTACTTTCC | SEQ ID NO: 5778 |
| rs2076175 | GCTTCTGCCCACTGTCCCCA | SEQ ID NO: 1047 | GCTTCTGCCCACTGTCCCCG | SEQ ID NO: 3413 | CTGTCACAGAGCAGGAAGAG | SEQ ID NO: 5779 |
| rs3871246 | GGGGAGCATCATGGCACACA | SEQ ID NO: 1048 | GGGGAGCATCATGGCACACG | SEQ ID NO: 3414 | CAACCAGAGCCAAGGTGAT | SEQ ID NO: 5780 |
| rs2281388 | ATCTGGGCCAGCCCAACACCT | SEQ ID NO: 1049 | ATCTGGGCCAGCCCAACACCC | SEQ ID NO: 3415 | CGTCTGCCATGTTGACAAGT | SEQ ID NO: 5781 |
| rs3763314 | TGAATGGGCTCATATAAGTTTCCTTAA | SEQ ID NO: 1050 | TGAATGGGCTCATATAAGTTTCCTTAG | SEQ ID NO: 3416 | CATATTTTTTATAAAGCAAATGGG | SEQ ID NO: 5782 |
| rs6913173 | CAATACACAAACCTTTACAGTATTCAGAT | SEQ ID NO: 1051 | CAATACACAAACCTTTACAGTATTCAGAC | SEQ ID NO: 3417 | TAGTCATGTCTAGATTTGAGGAGA | SEQ ID NO: 5783 |
| rs3873408 | CTCATGGGGGCAGGTGCAGT | SEQ ID NO: 1052 | CTCATGGGGGCAGGTGCAGC | SEQ ID NO: 3418 | ATTCCACCAGCTGTGTAGTTT | SEQ ID NO: 5784 |
| rs3997995 | AGAAGGCTGTGGGGGGCCA | SEQ ID NO: 1053 | AGAAGGCTGTGGGGGGCCG | SEQ ID NO: 3419 | ATTTGGTGTTGAGTCCATCAT | SEQ ID NO: 5785 |
| rs2523924 | CAATGGTACCTTCCATATAGCATATAGT | SEQ ID NO: 1054 | CAATGGTACCTTCCATATAGCATATAGTTC | SEQ ID NO: 3420 | TCAGAAAGTAACAAAAAGAAGAAG | SEQ ID NO: 5786 |
| rs2239517 | GGGGGAAGGTGCAGGCCA | SEQ ID NO: 1055 | GGGGGAAGGTGCAGGCCG | SEQ ID NO: 3421 | CTCTTCAGCTTCTCCTTGTTCT | SEQ ID NO: 5787 |
| rs3094225 | CTGCTGCAACTCAAGGCTCACA | SEQ ID NO: 1056 | CTGCTGCAACTCAAGGCTCACG | SEQ ID NO: 3422 | CTGTGACCGGTGGTGGTAG | SEQ ID NO: 5788 |
| rs3869117 | CATGTAAAATTTATCTTTAATTCCCAATGG | SEQ ID NO: 1057 | CATGTAAAATTTATCTTTAATTCCCAATGC | SEQ ID NO: 3423 | ATTTTATTAACTTGACTGTGGAATT | SEQ ID NO: 5789 |
| rs3734837 | TGGGCTTGAGTGGGCAGCTT | SEQ ID NO: 1058 | TGGGCTTGAGTGGGCAGCTC | SEQ ID NO: 3424 | AGTGGGTTCAAAGAGCTGAGT | SEQ ID NO: 5790 |
| rs2524180 | CTGTTAAAGCAACTGACTGCTAAAGAT | SEQ ID NO: 1059 | CTGAAAAAGCAACTGACTGCTAAAGAC | SEQ ID NO: 3425 | ATGAAGTCTCTGACGAAAAA | SEQ ID NO: 5791 |
| rs2844504 | ACCCCCACCCAGACCCAAA | SEQ ID NO: 1060 | ACCCCCACCCAGACCCAAC | SEQ ID NO: 3426 | CAGGCTCTAGTCCAACGG | SEQ ID NO: 5792 |
| rs2206618 | GCATTTCAGTGGTATTTTCCATTTTTGT | SEQ ID NO: 1061 | GCATTTCAGTGGTATTTTCCATTTTTGC | SEQ ID NO: 3427 | GCAACTCTATTAACATCTTGAT | SEQ ID NO: 5793 |
| rs406424 | AGGAAAGGGAGAGAGAGGAAAGAAAT | SEQ ID NO: 1062 | AGGAAAGGGAGAGAGAGGAAAGAAAG | SEQ ID NO: 3428 | AGGAAGGAAATCAAATTCATATC | SEQ ID NO: 5794 |
| rs3830038 | CTCTTCCCCCACCCCACTGAA | SEQ ID NO: 1063 | CTCTTCCCCCACCCCACTGAT | SEQ ID NO: 3429 | ATCCTCCTAAGGGAGCTGG | SEQ ID NO: 5795 |
| rs1048709 | ACCTGCCAAGTGAATGGCCGA | SEQ ID NO: 1064 | ACCTGCCAAGTGAATGGCCGG | SEQ ID NO: 3430 | AGACAGCGATCTGTGACAAC | SEQ ID NO: 5796 |
| rs3093661 | CACCCAAGGGGAAATGGAGACA | SEQ ID NO: 1065 | CACCCAAGGGGAAATGGAGACG | SEQ ID NO: 3431 | AGATGGGATGGGTGAAAGAT | SEQ ID NO: 5797 |
| rs2252856 | GAAGGGCCAACCCCCCCA | SEQ ID NO: 1066 | GAAGGGCCAACCCCCCCG | SEQ ID NO: 3432 | TAGGAGGTGCAGGGTAGGA | SEQ ID NO: 5798 |
| rs2857177 | CAAATCAATGGATTAAATAACCTAGTTGAA | SEQ ID NO: 1067 | CAAATCAATGGATTAAATAACCTAGTTGAG | SEQ ID NO: 3433 | TATTGGAAAGTAGATGAACAGTAATT | SEQ ID NO: 5799 |
| rs9968 | AGGGCCCCAGTGCCATCTC | SEQ ID NO: 1068 | AGGGCCCCAGTGCCATCTG | SEQ ID NO: 3434 | TTGGGCCTCAGAACTCTG | SEQ ID NO: 5800 |
| rs365052 | GTTCCGGATCTGGGCGCG | SEQ ID NO: 1069 | GTTCCGGATCTGGGCGCC | SEQ ID NO: 3435 | AATGACAAACTAAGCGCGG | SEQ ID NO: 5801 |
| rs1800624 | CCTTCATGATGCAGGCCCAAA | SEQ ID NO: 1070 | CCTTCATGATGCAGGCCCAAT | SEQ ID NO: 3436 | AGTCTGGCCTGAACCCTATT | SEQ ID NO: 5802 |
| rs2856715 | TGCAACACTCTCTCACTTTACCTTGTTT | SEQ ID NO: 1071 | TGCAACACTCTCTCACTTTACCTTGTTC | SEQ ID NO: 3437 | AGAGTTTGTAATTATATGTTTGTGG | SEQ ID NO: 5803 |
| rs3130444 | GTGAGACCCCATTCCTACAAAAT | SEQ ID NO: 1072 | GTGAGACCCCATTCCTACAAAAC | SEQ ID NO: 3438 | ATGCTGGTGTGTACCTGTAAAG | SEQ ID NO: 5804 |
| rs2071551 | CGCTGCAAGGTCGTGCGT | SEQ ID NO: 1073 | CGCTGCAAGGTCGTGCGC | SEQ ID NO: 3439 | GTAGTTGTGTCTGCACACCTT | SEQ ID NO: 5805 |
| rs2301221 | TTTCATCACACAGGTACTAAGCATTCTA | SEQ ID NO: 1074 | TTTCATCACACAGGTACTAAGCATTCTG | SEQ ID NO: 3440 | ACTTTTCTTACATTTATATTTTGATTT | SEQ ID NO: 5806 |
| rs6927322 | GGAGCCAGGGCTCAGCCAT | SEQ ID NO: 1075 | GGAGCCAGGGCTCAGCCAG | SEQ ID NO: 3441 | CTGATGACCTCTGTCAAAGCT | SEQ ID NO: 5807 |
| rs7768179 | GGAATTCCCGGATGAAAAGTGAAA | SEQ ID NO: 1076 | GGAATTCCCGGATGAAAAGTGAAG | SEQ ID NO: 3442 | GAGGGTTTCTTCCTGGTTT | SEQ ID NO: 5808 |
| rs6913646 | GTCTACACCAGCCATGTGGAA | SEQ ID NO: 1077 | GTCTACACCAGCCATGTGGAG | SEQ ID NO: 3443 | CTCCTCACAGTAGAGTGGTGTAG | SEQ ID NO: 5809 |
| rs2256820 | GCTTCTGCCCACTGTCCCCA | SEQ ID NO: 1078 | GCTTCTGCCCACTGTCCCCG | SEQ ID NO: 3444 | CCTTTCTGCCTGTTGAAGAG | SEQ ID NO: 5810 |
| rs4713410 | GAGGCATGGCACTGCCATGT | SEQ ID NO: 1079 | GAGGCATGGCACTGCCATGG | SEQ ID NO: 3445 | GGACCACCTGAGAAAGACA | SEQ ID NO: 5811 |
| rs872956 | TTTAGCATCTAACAATGTTCGACATATA | SEQ ID NO: 1080 | TTTAGCATCTAACAATGTTCGACATATT | SEQ ID NO: 3446 | TAGGCTTTTGATACTATTTTTTTACT | SEQ ID NO: 5812 |
| rs4959077 | CCAACTCACCCCACGTGGAT | SEQ ID NO: 1081 | CCAACTCACCCCACCTGGAC | SEQ ID NO: 3447 | CTAGTGAGCCCGAGAGACAG | SEQ ID NO: 5813 |
| rs2071282 | AGGAGGTGCCTTTGGGGCAGA | SEQ ID NO: 1082 | AGGAGGTGCCTTTGGGGCAGG | SEQ ID NO: 3448 | GAAGCACTCGTTGACATCAC | SEQ ID NO: 5814 |
| rs1264328 | AGAGGCCCCCTGTGTCCCT | SEQ ID NO: 1083 | AGAGGCCCCCTGTGTCCCC | SEQ ID NO: 3449 | CCAAATGACCCTCAACCATC | SEQ ID NO: 5815 |
| rs3918149 | GCGCCTGCGCTGTGTGTA | SEQ ID NO: 1084 | GCGCCTGCGCTGTGTGTG | SEQ ID NO: 3450 | ATGCTGAACTCGTATGGAGAG | SEQ ID NO: 5816 |
| rs2157051 | AAGAGTTCTGATGAGTGCAAAACAGA | SEQ ID NO: 1085 | AAGAGTTCTGATGAGTGCAATTTCAGG | SEQ ID NO: 3451 | CAAAATACTATTCTTATTGCAAGAA | SEQ ID NO: 5817 |
| rs7750514 | AGGGACGGGGCCCAAGCT | SEQ ID NO: 1086 | AGGGACGGGGCCCAAGCC | SEQ ID NO: 3452 | AGGGTTTCTTCCTGGTTTCTC | SEQ ID NO: 5818 |
| rs3128964 | TCAGCTGTCACATAATAAGCTAAGGAAGA | SEQ ID NO: 1087 | TCAGCTGTCACATAATAAGCTAAGGAAGG | SEQ ID NO: 3453 | AGGACCCTTTATCTGTCTTATTTT | SEQ ID NO: 5819 |
| rs6912877 | GAGTGAGCCATGTGGATCCCTA | SEQ ID NO: 1088 | GAGTGAGCCATGTGGATCCCTG | SEQ ID NO: 3454 | AAGTGCATGTGGGAGTACC | SEQ ID NO: 5820 |
| rs1541268 | TTTGCCACTGTTTGCTGTGGGT | SEQ ID NO: 1089 | TTTGCCACTGTTTGCTGTGGGC | SEQ ID NO: 3455 | GTGTGATCTTGTGAGATATATATATAA | SEQ ID NO: 5821 |
| rs6906175 | AGGGCAGGCAGGGCCCTGC | SEQ ID NO: 1090 | AGGGCAGGCAGGGCCCTGG | SEQ ID NO: 3456 | CGTGCTTATGAAGTTGGAGC | SEQ ID NO: 5822 |
| rs2074506 | GCCCCCTTCCATCCCCAGT | SEQ ID NO: 1091 | GCCCCCTTCCATCCCCAGG | SEQ ID NO: 3457 | GCTGCAGGAAAAGCTGAGA | SEQ ID NO: 5823 |
| rs2239523 | GGTAGAGGGAATGGGAGAGACCAGC | SEQ ID NO: 1092 | GGTAGAGGGAATGGGAGAGACCAGG | SEQ ID NO: 3458 | ACAGAGCTGCCTTTGTGAG | SEQ ID NO: 5824 |
| rs2242665 | GGAGCGCCGGTGGAGTAAT | SEQ ID NO: 1093 | GGAGCGCCGGTGGAGTAAC | SEQ ID NO: 3459 | TTGGTCCATGGAAAGCAG | SEQ ID NO: 5825 |
| rs3131300 | AGGGAGGGTCAGTGGGGTTGA | SEQ ID NO: 1094 | AGGGAGGGTCAGTGGGGTTGG | SEQ ID NO: 3460 | ACAGACTGAGGACCAGCAC | SEQ ID NO: 5826 |
| rs465223A | GGATCCCCGGCACCCAG | SEQ ID NO: 1095 | AGGATCCCCGGCACCCAC | SEQ ID NO: 3461 | TCTCTTCCACTGCATTCCC | SEQ ID NO: 5827 |
| rs3819299 | GGAGGAGCTCACCCACCCCA | SEQ ID NO: 1096 | GGAGGAGCTCACCCACCCCC | SEQ ID NO: 3462 | AATTCCTCCTGTCCCACGT | SEQ ID NO: 5828 |
| rs2844524 | TCGCTTACATAACCCAGGGCGT | SEQ ID NO: 1097 | TCGCTTACATAACCCAGGGCGG | SEQ ID NO: 3463 | AGTTTTTGCGACGGAGTTTT | SEQ ID NO: 5829 |
| rs6915440 | AGGGGCCTGGGGCAACGA | SEQ ID NO: 1098 | AGGGGCCTGGGGCAACGG | SEQ ID NO: 3464 | CATATCTCCCGAACCATATATTT | SEQ ID NO: 5830 |
| rs1614309 | CAACAAACATCATCTACCGCAGTTTT | SEQ ID NO: 1099 | CAACAAACATCATCTACCGGAGTTTC | SEQ ID NO: 3465 | AAGGTTTCCCTGAGTGCTTC | SEQ ID NO: 5831 |
| rs2076183 | GCCCTGACCGATACTTGGGCA | SEQ ID NO: 1100 | GCCCTGACCGATACTTGGGCG | SEQ ID NO: 3466 | AGAGAGAAACGGCCTCTGT | SEQ ID NO: 5832 |
| rs3763311 | TGGGCTTTTTGTTTTTTTAATTCATTCA | SEQ ID NO: 1101 | TGGGCTTTTTGTTTTTTTAATTCATTCG | SEQ ID NO: 3467 | GTTTTTAAGTCTATTCTAAAAGGCC | SEQ ID NO: 5833 |
| rs3097650 | TGAAGGTGGAAAACAAAAGAAGTACAT | SEQ ID NO: 1102 | TGAAGGTGGAAAACATTTAAGAAGTACAC | SEQ ID NO: 3468 | TTCTACAAGAGATATTTATGGAGGA | SEQ ID NO: 5834 |
| rs2743945 | ACCCTGGGCTGCTGCCCAA | SEQ ID NO: 1103 | ACCCTGGGCTGCTGCCCAC | SEQ ID NO: 3469 | CCATCCTGAGTCTTGTTTTTATT | SEQ ID NO: 5835 |
| rs1737087 | CCACAGGCCCCCTCAGGA | SEQ ID NO: 1104 | CCACAGGCCCCCTCAGGG | SEQ ID NO: 3470 | ATAGGGAGGAGGCCAATG | SEQ ID NO: 5836 |
| rs1811165 | CCCAGAGTCACAAACTGTTATGTCAA | SEQ ID NO: 1105 | CCCAGAGTCACAAACTGTTATGTCAG | SEQ ID NO: 3471 | TCAGTTAGAAGTTGTATTAGTCTGTT | SEQ ID NO: 5837 |
| rs3828913 | AGGGCGGGGCCACGTTCAT | SEQ ID NO: 1106 | AGGGCGGGGCCACGTTCAG | SEQ ID NO: 3472 | AGAGAAAATTCTAGCGGCCT | SEQ ID NO: 5838 |
| rs3763312 | TGAAAAATACAAACTTCAGAGTGAAAGAAA | SEQ ID NO: 1107 | TGAAAAATACAAACTTCAGAGTGAAAGAAG | SEQ ID NO: 3473 | CTAAGACTTATTATATGTGATGTGTCA | SEQ ID NO: 5839 |
| rs2269711 | CACCTCTCCCCACCAGGTGA | SEQ ID NO: 1108 | CACCTCTCCCCACCAGGTGT | SEQ ID NO: 3474 | GGAGAGGTGAGATGAGCAG | SEQ ID NO: 5840 |
| rs2243873 | TCTGATTAGCAGCAGTTATAAGTAGGGT | SEQ ID NO: 1109 | TCTGATTAGCAGCAGTTATAAGTAGGGG | SEQ ID NO: 3475 | TTTGTTTTTGAAGACAGAACAC | SEQ ID NO: 5841 |
| rs2072108 | GCAGGCTCTGGGTCATGATA | SEQ ID NO: 1110 | GCAGGCTCTGGGTCATGATG | SEQ ID NO: 3476 | CTGGGCTGGAGGACTAAAATA | SEQ ID NO: 5842 |
| rs7770882 | GCTGAAAATTTTTTGGAATATCAGTTGATA | SEQ ID NO: 1111 | GCTGAAAATTTTTTGGAATATCAGTTGATG | SEQ ID NO: 3477 | AACATGTACTTGTATGCAATTGC | SEQ ID NO: 5843 |
| rs1736935 | CAGAGGAAAGAATTCCAAAGCAGCA | SEQ ID NO: 1112 | CAGAGGAAAGAATTCCAAAGCAGCG | SEQ ID NO: 3478 | AACTCTTAGGTTTAAACACATT | SEQ ID NO: 5844 |
| rs3823420 | ACCCCACCCGACCCCCAAT | SEQ ID NO: 1113 | ACCCCACCCGACCCCCAAC | SEQ ID NO: 3479 | CTATGGCAATGATTTATTCATGT | SEQ ID NO: 5845 |
| rs2071571 | GGAAAGTCCGGGTGGCCTCA | SEQ ID NO: 1114 | GGAAAGTCCGGGTGGCCTCG | SEQ ID NO: 3480 | CAGCATAACCCGCTAAGTTG | SEQ ID NO: 5846 |
| rs3093726 | GACGTGGTGGCATGCACCT | SEQ ID NO: 1115 | GACGTGGTGGCATGCACCC | SEQ ID NO: 3481 | TAATCCTACCACTTTGCAAGG | SEQ ID NO: 5847 |
| rs973233 | GTCAGGGACAGGGTCGGGGT | SEQ ID NO: 1116 | GTCAGGGACAGGGTCGGGGG | SEQ ID NO: 3482 | GGGACTCAGGATGCTTGGT | SEQ ID NO: 5848 |
| rs1150772 | GTGAGGAGTGGGCAGCAAGGAC | SEQ ID NO: 1117 | GTGAGGAGTGGGCAGCAAGGAG | SEQ ID NO: 3483 | CACCTGCTCTGTCCTCATC | SEQ ID NO: 5849 |
| rs1264301 | GCCTGCGAGAGAAGCAGGCT | SEQ ID NO: 1118 | GCCTGCGAGAGAAGCAGGCG | SEQ ID NO: 3484 | CTGAGATAGCAGGGGAGAG | SEQ ID NO: 5850 |
| rs367398 | AGCCCCTTCTCCAAGCCCCA | SEQ ID NO: 1119 | AGCCCCITCTCCAAGCCCCG | SEQ ID NO: 3485 | TCCCTGTCCCTCTTCAGG | SEQ ID NO: 5851 |
| rs241451 | CACCCCACCCCACCTCTCT | SEQ ID NO: 1120 | CACCCCACCCCACCTCTCC | SEQ ID NO: 3486 | ACACAGTGTCCAAATCTCCAT | SEQ ID NO: 5852 |
| rs2076528 | TGTTAAATAGAGTGGACAAAACACAATGAA | SEQ ID NO: 1121 | TGTTAAATAGAGTGGACAAAACACAATGAC | SEQ ID NO: 3487 | ATGTCATCTCTAAGAACAGCTCC | SEQ ID NO: 5853 |
| rs3115615 | TCTGCTGTGCAAGATAATTTCATCTTC | SEQ ID NO: 1122 | TCTGCTGTGCAAGATAATTTCATCTTG | SEQ ID NO: 3488 | AAAGATTTGATGTTACATTTTAC | SEQ ID NO: 5854 |
| rs2228088 | CCCCAGGGCTCCAGGCGT | SEQ ID NO: 1123 | CCCCAGGGCTCCAGGCGG | SEQ ID ND: 3489 | GCTTGTTCCTCAGCCTCTT | SEQ ID NO: 5855 |
| rs2269611 | ACTGCCCCCCTCCCATCCT | SEQ ID NO: 1124 | ACTGCCCCCCTCCCATCCC | SEQ ID NO: 3490 | ACCATCATCGCACAGCTC | SEQ ID NO: 5856 |
| rs408036 | GCTGGGGATTGGCAGCCA | SEQ ID NO: 1125 | GCTGGGGATTGGCAGCCG | SEQ ID NO: 3491 | AGGTGTGTCTTGCTCCATTT | SEQ ID NO: 5857 |
| rs3130998 | CCGCCCACGTGGACTGGT | SEQ ID NO: 1126 | CCGCCCACGTGGACTGGC | SEQ ID NO: 3492 | CTCCTGGCTACTCTCTCCTGT | SEQ ID NO: 5858 |
| rs2022537 | AAAGGTACAAAAAGTCACAAATTGAAACT | SEQ ID NO: 1127 | AAAGGTACAAAAAGTCACAAATTGAAACC | SEQ ID NO: 3493 | GAAAAAACTGAAGAAACAAAATTTT | SEQ ID NO: 5859 |
| rs2239520 | ACCATGCCGTCCCCAGAGGT | SEQ ID NO: 1128 | ACCATGCCGTCCCCAGAGGC | SEQ ID NO: 3494 | GAGTTGGTTTTGGGAATGTG | SEQ ID NO: 5860 |
| rs4993139 | TTCATGCATTTTACATCTAGATCTTCACA | SEQ ID NO: 1129 | TTCATGCATTTTACATCTAGATCTTCACG | SEQ ID NO: 3495 | TGACTTGTTTAATGTTACAAAAGG | SEQ ID NO: 5861 |
| rs3094215 | GGCCCATCGTCTCGCACTCT | SEQ ID NO: 1130 | GGCCCATCGTCTCGCACTCC | SEQ ID NO: 3496 | AGCTCCCACTCTGTGTCAG | SEQ ID NO: 5862 |
| rs1063379 | CCCTAGGCCCCTTGTCATCTTCA | SEQ ID NO: 1131 | CCCTAGGCCCCTTGTCATCTTCG | SEQ ID NO: 3497 | CTCGATGCTTCTCTGCCTT | SEQ ID NO: 5863 |
| rs259942 | TTGCTGTTCTCATGATAGTGAATTTTCA | SEQ ID NO: 1132 | TTGCTGTTCTCATGATAGTGAATTTTCG | SEQ ID NO: 3498 | TGAGATTTTGTAGTGAGATTTGG | SEQ ID NO: 5864 |
| rs3734861 | TGTATCCCTTCCTTCTCTCCTAGTACCT | SEQ ID NO: 1133 | TGTATCCCTTCCTTCTCTCCTAGTACCC | SEQ ID NO: 3499 | TAAAGCAAATGATTAGCCCC | SEQ ID NO: 5865 |
| rs2499 | TCTTCCTTCCCTATTAAAATTAGAACCTT | SEQ ID NO: 1134 | TCTTCCTTCCCTATTAAAATTAGAACCTG | SEQ ID NO: 3500 | GTATAAATTTACTTTCTCAAATTCTTG | SEQ ID NO: 5866 |
| rs4713250 | GTCTTCAATGATTTCAGCTGGGCA | SEQ ID NO: 1135 | GTCTTCAATGATTTCACCTGGGCC | SEQ ID NO: 3501 | GTTGTTTGTTTTGAGCTGCTG | SEQ ID NO: 5867 |
| rs2269426 | CTGGAGCCCCCAGAGCCTA | SEQ ID NO: 1136 | CTGGAGCCCCCAGAGCCTG | SEQ ID NO: 3502 | GCTCAGCTGCTCTAGCCC | SEQ ID NO: 5858 |
| rs887467 | GGAAGGGAAGGCTGGGCTGAC | SEQ ID NO: 1137 | GGAAGGGAAGGCTGGGCTGAG | SEQ ID NO: 3503 | CTGCTCATCTCACCTCTCC | SEQ ID NO: 5869 |
| rs2269712 | ACAAGGGGTGGGTGCCTGGA | SEQ ID NO: 1138 | ACAAGGGGTGGGTGCCTGGG | SEQ ID NO: 3504 | CTGGATATGAGCCCAGTCAG | SEQ ID NO: 5870 |
| rs2302396 | GGCCACACCAACTTCCCCT | SEQ ID NO: 1139 | GGCCACACCAACTTCCCCC | SEQ ID NO: 3505 | CTCCACCTCTCCTCCTCTC | SEQ ID NO: 5871 |
| rs2008560 | GGCTAGGGCCGTGCTGGC | SEQ ID NO: 1140 | GGCTAGGGCCGTGCTGGG | SEQ ID NO: 3506 | ATTCTCCGCAGAGGATTTCT | SEQ ID NO: 5872 |
| rs1480383 | TCCAGAAAAAAAATTATTGAAAAGAGCA | SEQ ID NO: 1141 | TCCAGAAAAAAAATTATTGAAAAGAGCC | SEQ ID ND: 3507 | TAAATATACAAAATATCAAAGCAGTC | SEQ ID NO: 5873 |
| rs3129997 | ATGGGCCACCTGGGTGCCG | SEQ ID NO: 1142 | ATGGGCCACCTGGGTGCCC | SEQ ID NO: 3508 | CTTTGTGCTCCATGTCATCT | SEQ ID NO: 5874 |
| rs2517588 | TGCAGGTTATAACCAGCATTTTTAAAAT | SEQ ID NO: 1143 | TGCAGGTTATAACCAGCATTTTTAAAAC | SEQ ID NO: 3509 | GACTGTAATTTAAAAATCAGAATATAT | SEQ ID NO: 5875 |
| rs465474 | CACTTGTTTTCCGTCGACGTGATT | SEQ ID NO: 1144 | CACTTGTTTTCCGTCGACGTGATC | SEQ ID NO: 3510 | CTCCTGGTCGTGGTTGACT | SEQ ID NO: 5876 |
| rs3218812 | CCGCCTGGGAGCAGCTGT | SEQ ID NO: 1145 | CCGCCTGGGAGCAGCTGG | SEQ ID NO: 3511 | CTCAGCAGCCCTGTACTTTC | SEQ ID NO: 5877 |
| rs2523791 | GCAGCAGCTCAAAACAAACAACAT | SEQ ID NO: 1146 | GCAGCAGCTCAAAACAAACAACAC | SEQ ID NO: 3512 | CCCAGGTGAAATCATTGAAG | SEQ ID NO: 5878 |
| rs3828912 | ACCTGGCGGCCAGAAAAGGT | SEQ ID NO: 1147 | ACCTGGCGGCCAGAAAAGGG | SEQ ID NO: 3513 | AGCCAAAATCAAGGACCAG | SEQ ID NO: 5879 |
| rs2517444 | CCACGCCCGGCCTCTTCT | SEQ ID NO: 1148 | CCACGCCCGGCCTCTTCC | SEQ ID NO: 3514 | TTCTTCCTGCTATATCTATCGTTT | SEQ ID NO: 5880 |
| rs2257347 | GACCCATGGCCTGCCCCA | SEQ ID NO: 1149 | GACCCATGGCCTGCCCCG | SEQ ID NO: 3515 | GTAGCAGTGGCTGTAGGGAG | SEQ ID NO: 5881 |
| rs364950 | GGCGTCTGCCTGAGTTTGGT | SEQ ID NO: 1150 | GGCGTCTGCCTGAGTTTGGC | SEQ ID NO: 3516 | ACTTTGCCCGCTTTGAC | SEQ ID NO: 5882 |
| rs2006164 | GCCGCAGGAAGCTTCAGGT | SEQ ID NO: 1151 | GCCGCAGGAAGCTTCAGGC | SEQ ID NO: 3517 | TGGCCTGGTGGAGTTAG | SEQ ID NO: 5883 |
| rs2857172 | TGAAAAGTAGATAGTTGAGTAAAGCTCATT | SEQ ID NO: 1152 | TGAAAAGTAGATAGTTGAGTAAAGCTCATC | SEQ ID NO: 3518 | AATTATTTAATCAGCTACTTCCATT | SEQ ID NO: 5884 |
| rs3873243 | TTTAGCCAGGGTAACAGAGTGACGA | SEQ ID NO: 1153 | TTTAGCCAGGGTAACAGAGTGACGC | SEQ ID NO: 3519 | ATAAAAAAATTAATAATAAAGGGAGTG | SEQ ID NO: 5885 |
| rs3763327 | TCTGTTGTTTTTCTCAAAATTTCTATAGAC | SEQ ID NO: 1154 | TCTGTTGTTTTTCTCAAAATTTCTATAGAG | SEQ ID NO: 3520 | GATGATAAAAATCTAAGAGAAGCTAA | SEQ ID NO: 5886 |
| rs1061801 | CGGGGGTGGCGGGGAGAA | SEQ ID NO: 1155 | CGGGGGTGGCGGGGAGAG | SEQ ID NO: 3521 | CAGGGTAGAAATAAAGAGCGC | SEQ ID NO: 5887 |
| rs4713519 | TGTGGTAAGCACTTAATAATTATCAGCTA | SEQ ID NO: 1156 | TGTGGTAAGCACTTAATAATTATCAGCTC | SEQ ID NO: 3522 | TACTGTTATTACTTTTGCATAATAAGA | SEQ ID NO: 5888 |
| rs909722 | CTCTTGGACCGCAGCGGAT | SEQ ID NO: 1157 | CTCTTGGACCGCAGCGGAA | SEQ ID NO: 3523 | AGGAGGAACATGCAGAGCT | SEQ ID NO: 5889 |
| rs2213569 | AAATTGCTAAGACAGATTTTAAATATTCAT | SEQ ID NO: 1158 | AAATTGCTAAGACAGATTTTAAATATTCAC | SEQ ID NO: 3524 | CCACATAAAAAAATAAGTATGTGAA | SEQ ID NO: 5890 |
| rs644827 | CTGCCGCAGGAAGATGAGCAT | SEQ ID NO: 1159 | CTGCCGCAGGAAGATGAGCAC | SEQ ID NO: 3525 | AGCAGCAGGATGGCTTC | SEQ ID NO: 5891 |
| rs3763341 | AAATGCACCACTCTTAGTTATGTATAGGT | SEQ ID NO: 1160 | AAATGCACCACTCTTAGTTATGTATAGGC | SEQ ID NO: 3526 | AGTCCAAAGATAAAGAAAACC | SEQ ID NO: 5892 |
| rs2254287 | AGGGGCTCGGGAGCTGGAC | SEQ ID NO: 1161 | AGGGGCTCGGGAGCTGGAG | SEQ ID NO: 3527 | AGGGAAGGCAGTGAAGAGAG | SEQ ID NO: 5893 |
| rs769178 | GAGGGTGACCCAAGGCCCTATA | SEQ ID NO: 1162 | GAGGGTGACCCAAGGCCCTATC | SEQ ID NO: 3528 | AGGTCACCCCAAAGGGT | SEQ ID NO: 5894 |
| rs2270190 | GCCCAGGTGTCCTGGCTCA | SEQ ID NO: 1163 | GCCCAGGTGTCCTGGCTCG | SEQ ID NO: 3529 | AGCTCATCCCTGGTTGC | SEQ ID NO: 5895 |
| rs986521 | CCGGGCAATGAGATACCACACA | SEQ ID NO: 1164 | CCGGGCAATGAGATACCACACG | SEQ ID NO: 3530 | CCTTAAACCCACCAGCTC | SEQ ID NO: 5896 |
| rs206775 | TTCAAGTATTTTTGTGTCCTTGTACAGTAA | SEQ ID NO: 1165 | TTCAAGTATTTTTGTGTCCTTGTACAGTAC | SEQ ID NO: 3531 | AATATTTCTTTCATCTCTTTGCC | SEQ ID NO: 5897 |
| rs241447 | GTGGGCGCGCTGAACTGT | SEQ ID NO: 1166 | GTGGGCGCGCTGAACTGC | SEQ ID NO: 3532 | GAGCAATCACCAGCACTGT | SEQ ID NO: 5898 |
| rs2157336 | TTTTTATTGTTTTTTCAGTGTGGCA | SEQ ID NO: 1167 | TTTTTATTGTTTTTTCAGTGTGGCG | SEQ ID NO: 3533 | TTATTTACAAACTCTGACTTATAATG | SEQ ID NO: 5899 |
| rs1044507 | GGGGGCGGGGCCTATTCT | SEQ ID NO: 1168 | GGGGGCGGGGCCTATTCG | SEQ ID NO: 3534 | GAGTAGGAGCAGGAGGAGG | SEQ ID NO: 5900 |
| rs3094204 | CACCTCCTGGGTAGCTGGGACTAT | SEQ ID NO: 1169 | CACCTCCTGGGTAGCTGGGACTAC | SEQ ID NO: 3535 | CCCTTTGTGATCATTCAAAG | SEQ ID NO: 5901 |
| rs1611613 | CCGACGGGCGCTTCCTCT | SEQ ID NO: 1170 | CCGACGGGCGCTTCCTCC | SEQ ID NO: 3536 | GGTATGAACAGCACGCCTAC | SEQ ID NO: 5902 |
| rs1736927 | GTGAGTAACTCCGGCCCAGGGA | SEQ ID NO: 1171 | GTGAGTAACTCCGGCCCAGGGC | SEQ ID NO: 3537 | CAGATCACGACCCCCAC | SEQ ID NO: 5903 |
| rs2855438 | ACAAGGGCAGAGGGGAGCTGA | SEQ ID NO: 1172 | ACAAGGGCAGAGGGGAGCTGT | SEQ ID NO: 3538 | GGAGGACCAGAGGCTGCT | SEQ ID NO: 5904 |
| rs3819284 | ACACAGGGTCCCAGGCTGCA | SEQ ID NO: 1173 | ACACAGGGTCCCAGGCTGCG | SEQ ID NO: 3539 | TAGCCCCTGTGTGGATG | SEQ ID NO: 5905 |
| rs6913471 | GAGTGAAACTCTATCTTCAAAAAAAATTA | SEQ ID NO: 1174 | GAGTGAAACTCTATCTTCAAAAAAAATTT | SEQ ID NO: 3540 | AAAAAAGAGTTACAAAGATTAATGA | SEQ ID NO: 5906 |
| rs969892 | CAAGGAATTTAGAAAATCATGCATATGTTA | SEQ ID NO: 1175 | CAAGGAATTTAGAAAATCATGCATATGTTG | SEQ ID NO: 3541 | TTTTTTAAATGTAATTTGAAAATCTT | SEQ ID NO: 5907 |
| rs2516507 | TGTTGGTATTTTAATAAAAATTATTTGGCAT | SEQ ID NO: 1176 | TGTTGGTATTTTAATAAAAATTATTGGCAC | SEQ ID NO: 3542 | TAATATTTATACATACTTTGGGGTACA | SEQ ID NO: 5908 |
| rs2071552 | AGACCGCAGCTCCGGGGA | SEQ ID NO: 1177 | AGACCGCAGCTCCGGGGG | SEQ ID NO: 3543 | TTCTGCTTCAGCGCTGAG | SEQ ID NO: 5909 |
| rs375256 | CGCTTTGACCCGCAGGGT | SEQ ID NO: 1178 | CGCTTTGACCCGCAGGGC | SEQ ID NO: 3544 | AATCAAAGCCCATCTGGAC | SEQ ID NO: 5910 |
| rs2074465 | CGTTACCATCTTCCGGATCCCA | SEQ ID NO: 1179 | CGTTACCATCTTCCGGATCCCG | SEQ ID NO: 3545 | CCTTCTCCACCTGCTCCT | SEQ ID NO: 5911 |
| rs3130712 | TTCAGAGTATTAAGAGACAGAGTTAAGCCA | SEQ ID NO: 1180 | TTCAGAGTATTAAGAGACAGAGTTAAGCCG | SEQ ID NO: 3546 | TGTTTTACTCTTTAGAAAACGCA | SEQ ID NO: 5912 |
| rs2286654 | GGGCCCCCACCCATGCCT | SEQ ID NO: 1181 | GGGCCCCCACCCATGCCC | SEQ ID NO: 3547 | CTCCCAAGGATCTTTAGAGATTA | SEQ ID NO: 5913 |
| rs1265089 | GCAGGCAGCGGCTGTGGT | SEQ ID NO: 1182 | GCAGGCAGCGGCTGTGGC | SEQ ID NO: 3548 | CATCAGCGTACTTGGACTG | SEQ ID NO: 5914 |
| rs915652 | CTGTGGGCAGGCCTGACCT | SEQ ID NO: 1183 | CTGTGGGCAGGCCTGACCC | SEQ ID NO: 3549 | GTGTGGGGTTGAAATCCAG | SEQ ID NO: 5915 |
| rs362513 | TGTGCAGTTCTTGAAATTTAAGTACTCACT | SEQ ID NO: 1184 | TGTGCAGTTCTTGAAATTTAAGTACTCACC | SEQ ID NO: 3550 | AATATTACTTTTCCTTTTTTTGTTATT | SEQ ID NO: 5916 |
| rs3736710 | GAATAGACTAAGACAATAAGCAATTTTGCT | SEQ ID NO: 1185 | GAATAGACTAAGACAATAAGCAATTTTGCC | SEQ ID NO: 3551 | CTGCTTGTTAGTCTTATGAGAAGA | SEQ ID NO: 5917 |
| rs3130662 | GGTATGTTTTCCTTTTTTTCCAGGATTT | SEQ ID NO: 1186 | GGTATGTTTTCCTTTTTTTCCAGGATTG | SEQ ID NO: 3552 | TATTTTATTGTGTATTGTTGTTGTTG | SEQ ID NO: 5918 |
| rs2524056 | TCATTAGCCTTTGTGTCATAAAATAAAATA | SEQ ID NO: 1187 | TCATTAGCCTTTGTGTGATAAAATAAAATC | SEQ ID NO: 3553 | TGTGTAAAATTTGTGTCCTTTTAT | SEQ ID NO: 5919 |
| rs2395039 | ACTGCAGCCTGGAACTCCCT | SEQ ID NO: 1188 | ACTGCAGCCTGGAACTCCCC | SEQ ID NO: 3554 | CTCCAATTCCCAAAGAGCT | SEQ ID NO: 5920 |
| rs683208 | AGGGGGCCGGCCATGGAA | SEQ ID NO: 1189 | AGGGGGCCGGCCATGGAG | SEQ ID NO: 3555 | CTGAGAAAATACTAGGCCTTAGG | SEQ ID NO: 5921 |
| rs1264320 | AGGAGGGTCCCACCCTAGCCA | SEQ ID NO: 1190 | AGGAGGGTCCCACCCTAGCCG | SEQ ID NO: 3556 | AGAAGGGTGCAGAGGGTC | SEQ ID NO: 5922 |
| rs2074492 | CTCGGCCTCTGGGGAGAATGT | SEQ ID NO: 1191 | CTCGGCCTCTGGGGAGAATGC | SEQ ID NO: 3557 | AGTCCGGGTGGGTGACT | SEQ ID NO: 5923 |
| rs4524656 | AAATTTTTTTGTCAGCTAAAAATGTAGC | SEQ ID NO: 1192 | AAATTTTTTTGTCAGCTAAAAATGTAGG | SEQ ID NO: 3558 | TCATTATTGACATTACTATACATAGGG | SEQ ID NO: 5924 |
| rs3130934 | TATGTGACCCTGGGCAAGCTTTTT | SEQ ID NO: 1193 | TATGTGACCCTGGGCAAGCTTTTG | SEQ ID NO: 3559 | TTTGTTTGTTTGAGATGGAGT | SEQ ID NO: 5925 |
| rs1264457 | GCGAGCTGGGGCCCGACA | SEQ ID NO: 1194 | GCGAGCTGGGGCCCGACG | SEQ ID NO: 3560 | GTATGAACAGTTCGCCTACGA | SEQ ID NO: 5926 |
| rs2242667 | GCTTTGGCCATGCCGGTT | SEQ ID NO: 1195 | GCTTTGGCCATGCCGGTG | SEQ ID NO: 3561 | CCTGCTCTCTGTCGGCTC | SEQ ID NO: 5927 |
| rs7739536 | CAACTCTATGTTTCTTTGATTCTAACACAA | SEQ ID NO: 1196 | CAACTCTATGTTTCTTTGATTCTAACACAC | SEQ ID NO: 3562 | TAATTAAGTGACATGATTTTTACTAA | SEQ ID NO: 5928 |
| rs409715 | GCCGTGGTGATGCACACCT | SEQ ID NO: 1197 | GCCGTGGTGATGCACACCC | SEQ ID NO: 3563 | TTGCCCCAGCTACTTGG | SEQ ID NO: 5929 |
| rs3828914 | GTGGCCCCGCCCTCTCCAT | SEQ ID NO: 1198 | GTGGCCCCGCCCTCTCCAC | SEQ ID NO: 3564 | GCCTCAGTTTTCACTGGATAA | SEQ ID NO: 5930 |
| rs2272991 | TGGGAGCATGCAAGAACAAAGA | SEQ ID NO: 1199 | TGGGAGCATGCAAGAACAAAGG | SEQ ID NO: 3565 | AACACAGCAATCCAGGCAG | SEQ ID NO: 5931 |
| rs1362061 | TTGTTTTTGTTTCTGTAAAAGCACTTATC | SEQ ID NO: 1200 | TTGTTTTTGTTTCTGTAAAAGCACTTATG | SEQ ID NO: 3566 | TCTTGTTAACATATTTTAAAGTGCA | SEQ ID NO: 5932 |
| rs3132377 | AAACCTTACAACATATACTCCTTTCCATAT | SEQ ID NO: 1201 | AAACCTTACAACATATACTCCTTTCCATAC | SEQ ID NO: 3567 | TTCTAGTTTCTTTAACCAACCCT | SEQ ID NO: 5933 |
| rs743771 | CTCTACCACCCCTGGCCT | SEQ ID NO: 1202 | CTCTACCACCCCTGGCCC | SEQ ID NO: 3568 | TCCAGTTCTGTTGCAGTATTTAT | SEQ ID NO: 5934 |
| rs2523612 | GGGGAGGGGGTTGCTGTA | SEQ ID NO: 1203 | GGGGAGGGGGTTGCTGTC | SEQ ID NO: 3569 | AAAGAGGGATGGGGAGG | SEQ ID NO: 5935 |
| rs4713220 | TGACAGACTGTAATGCATGTAATATGATGT | SEQ ID NO: 1204 | TGACAGACTGTAATGCATGTAATATGATGA | SEQ ID NO: 3570 | ATACAGGTGAAATACAAAAAAT | SEQ ID NO: 5936 |
| rs2301224 | AGTTTCTGTCTTAGTATGAACATGAAAA | SEQ ID NO: 1205 | AGTTTGTGTCTTAGTTTATGAACATGAAAC | SEQ ID NO: 3571 | GTAGAATGTATAGCTTTGTTGATAA | SEQ ID NO: 5937 |
| rs3106191 | CGGCGCTGCGGCAATCCT | SEQ ID NO: 1206 | CGGCGCTGCGGCAATCCG | SEQ ID NO: 3572 | AAAGCAAAGGCTCTTGCACT | SEQ ID NO: 5938 |
| rs3749982 | GAGGCGCGAACACGGAGGA | SEQ ID NO: 1207 | GAGGCGCGAACACGGAGGT | SEQ ID NO: 3573 | CCCCTCAGGCATACTGC | SEQ ID NO: 5939 |
| rs1610678 | AGCTCGAATTGAGAAACTAGTTTTTTCA | SEQ ID NO: 1208 | AGCTCGAATTGAGAAACTAGTTTTTTCG | SEQ ID NO: 3574 | GCATTCTATAATACACTTAACATCTTT | SEQ ID NO: 5940 |
| rs2524055 | TTTGTATTAAAGGACACAAATTTTAGACAT | SEQ ID NO: 1209 | TTTGTATTAAAGGACACTTAATTTTAGACAG | SEQ ID NO: 3575 | TTTTATTTTATGACACAAAGGCT | SEQ ID NO: 5941 |
| rs2073721 | CAGAAGGCGTGGTCCCCAT | SEQ ID NO: 1210 | CAGAAGGCGTGGTCCCCAC | SEQ ID NO: 3576 | GAACAGGCAGGCTCTTTG | SEQ ID NO: 5942 |
| rs3132496 | GATTATGAATTATCAGGGATTTCTAAGAAA | SEQ ID NO: 1211 | GATTATGAATTATCAGGGATTTCTAAGAAC | SEQ ID NO: 3577 | TGACAAGTGTAGATGATAAAGGG | SEQ ID NO: 5943 |
| rs2071653 | CAGGCACTATTCTAACTACTTTACAAAAAT | SEQ ID NO: 1212 | CAGGCACTATTCTAACTACTTTACAAAAAC | SEQ ID NO: 3578 | TTATCTTATTCTGTTTAACTCCTTATG | SEQ ID NO: 5944 |
| rs7772982 | CATTTTAAATTCACAAAATATTTCTTCACA | SEQ ID NO: 1213 | CATTTTAAAACACAAAATATTTCTTCACG | SEQ ID NO: 3579 | AATATTACACCATTTAAGATGTATTGC | SEQ ID NO: 5945 |
| rs2524094 | GGGGAGGCGCCGCGTTGA | SEQ ID NO: 1214 | GGGGAGGCGCCGCGTTGG | SEQ ID NO: 3580 | GATTCTCCACTCCCCTGAGT | SEQ ID NO: 5946 |
| rs2252863 | GGCCTGTTACCCCCAGGCA | SEQ ID NO: 1215 | GGCCTGTTACCCCCAGGCG | SEQ ID NO: 3581 | CCAGGCTCCACTCAGTGAC | SEQ ID NO: 5947 |
| rs2532939 | GGGCTGGTGGGGAAAAGAGG | SEQ ID NO: 1216 | GGGCTGGTGGGGAAAAGAGC | SEQ ID NO: 3582 | GAGCCTGAAGGGCCAAC | SEQ ID NO: 5948 |
| rs3129793 | TGAGATATCTGCTGTACAAGGGATCAT | SEQ ID NO: 1217 | TGAGATATCTGCTGTACAAGGGATCAC | SEQ ID NO: 3583 | AATGAGAATTAATAATATAGAACATAA | SEQ ID NO: 5949 |
| rs3130926 | CCTGTCTTGAATCTTATTTTTTTCATTTAT | SEQ ID NO: 1218 | CCTGTCTTGAATCTTATTTTTTTCATTTAC | SEQ ID NO: 3584 | TTTTTTAAGAGAGGAGTCTTGCT | SEQ ID NO: 5950 |
| rs707918 | TGGACACTTGAGAAGTTATACAATTAGCCT | SEQ ID NO: 1219 | TGGACACTTGAGAAGTTATACAATTAGCCA | SEQ ID NO: 3585 | TATTAATTATTAGGAATAATCCATTCA | SEQ ID NO: 5951 |
| rs6925178 | GCGGGTCTTTGGGGCCAT | SEQ ID NO: 1220 | GCGGGTCTTTGGGGCCAG | SEQ ID NO: 3586 | TTTGAGGAGGGAGTCGG | SEQ ID NO: 5952 |
| rs6456902 | AACAGAAAATTTACTATTGGTGGACAAA | SEQ ID NO: 1221 | AACAGAAAAAAACTATTGGTGGTTCAAG | SEQ ID NO: 3587 | TTATAACATGAAATTATTGAATTTCA | SEQ ID NO: 5953 |
| rs1610682 | TGTGCTTTCTCAGATAATAAAAGAAATAAT | SEQ ID NO: 1222 | TGTGCTTTCTCAGATAATTAAAGAAATAAC | SEQ ID NO: 3588 | AACACTTACTGAGAAGACCACAA | SEQ ID NO: 5954 |
| rs2073720 | CCAGCTGCTGGGCCACCT | SEQ ID NO: 1223 | CCAGCTGCTGGGCCACCC | SEQ ID NO: 3589 | CTGCCTCCCCTAAAAGGAG | SEQ ID NO: 5955 |
| rs2071785 | TTCCTTTTGTTGTCTTACTCATTTTGGA | SEQ ID NO: 1224 | TTCCTTTGTTGTCTTACTCATTTTGGG | SEQ ID NO: 3590 | CAAGAAGAAGTAAAATGGC | SEQ ID NO: 5956 |
| rs2856993 | GGTGTCTTTTGCATATTGCAAATAATAC | SEQ ID NO: 1225 | GGTGTCTTTTGCATATTGCAAATAATAG | SEQ ID NO: 3591 | GTTTTTATCTTAATCTAATTTAAAAAT | SEQ ID NO: 5957 |
| rs4959012 | CTGGCCCAGCCACTGCTGA | SEQ ID NO: 1226 | CTGGCCCAGCCACTGCTGG | SEQ ID NO: 3592 | CTGTTCCCTGGTTTGGTG | SEQ ID NO: 5958 |
| rs614549 | TTGAGATACTCCTTACTGTACTTAAATTGA | SEQ ID NO: 1227 | TTGAGATACTCCTTACTGTACTTAAATTGG | SEQ ID NO: 3593 | AACTGGATATCTCATCTTATGCTA | SEQ ID NO: 5959 |
| rs3749963 | GGGGGCTCGGGGGTTGCA | SEQ ID NO: 1228 | GGGGGCTCGGGGGTTGCG | SEQ ID NO: 3594 | AGGTTCTGAAGGCTTCTCCT | SEQ ID NO: 5960 |
| rs3757324 | TTTTTCATTTTATAATATTCCATGATGCT | SEQ ID NO: 1229 | TTTTTCATTTTATTATATTCCATGATGCG | SEQ ID NO: 3595 | TTAAATTTTTATACAATCATATTTCA | SEQ ID NO: 5961 |
| rs6457150 | CCAGGCTCCCCCATCCAG | SEQ ID NO: 1230 | CCAGGCTCCCCCATCCAC | SEQ ID NO: 3596 | GCCGCCGATTACATAAAC | SEQ ID NO: 5962 |
| rs915651 | CCATGCCCCAGCCACGCA | SEQ ID NO: 1231 | CCATGCCCCAGCCACGCG | SEQ ID NO: 3597 | ACAGGACGGTAGGAGAGGAG | SEQ ID NO: 5963 |
| rs3130456 | GTTCCCGATTGGCCAGCTCATT | SEQ ID NO: 1232 | GTTCCCGATTGGCCAGCTCATG | SEQ ID NO: 3598 | GTTCTCTTCCGCGCCATT | SEQ ID NO: 5964 |
| rs1150739 | CACAAAATCCGCAGATATCAGAATTATA | SEQ ID NO: 1233 | CACAAAATCCGCAGATATCAGAATTATG | SEQ ID NO: 3599 | GACATAGAATATAATAACCATATTTAA | SEQ ID NO: 5965 |
| rs1611617 | ACAGGGCGCCTCCCGGATC | SEQ ID NO: 1234 | ACAGGGCGCCTCCCGGATG | SEQ ID NO: 3600 | CCTGTAGATCTCCGGGG | SEQ ID NO: 5966 |
| rs3094139 | TGTATAAATAGTCCATTCCTTTTTATTGCT | SEQ ID NO: 1235 | TGTATAAATAGTCCATTCCTTTTTATTGCC | SEQ ID NO: 3601 | TGTATGGATGTACTACAATGTGTC | SEQ ID NO: 5967 |
| rs2394414 | AAAAACAAACAAGGGGAGGGGTA | SEQ ID NO: 1236 | AAAAACAAACAAGGGGAGGGGTG | SEQ ID NO: 3602 | AAGAAGAAGAAAAACAAAAACA | SEQ ID NO: 5968 |
| rs11724 | CAGGAGGACGCATTCGGCG | SEQ ID NO: 1237 | CAGGAGGACGCATTCGGCC | SEQ ID NO: 3603 | AACCCAGAGCCAGCGAT | SEQ ID NO: 5969 |
| rs1611185 | AGCTCTGCATGTTCCTCCTCCTA | SEQ ID NO: 1238 | AGCTCTGCATGTTCCTCCTCCAG | SEQ ID NO: 3604 | ACATCTGAGCCGCCTT | SEQ ID NO: 5970 |
| rs6940440 | TTAAACACAAGGATTTTTCTTTATTTTTT | SEQ ID NO: 1239 | TTAAACACAAGGATTTTTCTTTATTTTTG | SEQ ID NO: 3605 | TTTTTCATAGTTCATTTAAACATAAA | SEQ ID NO: 5971 |
| rs7452208 | GAAGGCCTTTGTTACTTACTTAGCTCCA | SEQ ID NO: 1240 | GAAGGCCTTTGTTACTTACTTAGCTCCG | SEQ ID NO: 3606 | CAGGAATAGCCCTTGGGTT | SEQ ID NO: 5972 |
| rs3763342 | CATGTGGATTTTGTCATTCTAATTTATTAT | SEQ ID NO: 1241 | CATGTGGATTTTGTCATTCTAAAAATTAC | SEQ ID NO: 3607 | GTTTTCTTTTATCTTTGGACTG | SEQ ID NO: 5973 |
| rs2518030 | GCTCCTTCCCGCAGAGGCT | SEQ ID NO: 1242 | GCTCCTTCCCGCAGAGGCC | SEQ ID NO: 3608 | ACTCACCTGCCCAGGTCT | SEQ ID NO: 5974 |
| rs7772174 | ACGATGACTTCCAGCTTCATCCAA | SEQ ID NO: 1243 | ACGATGACTTCCAGCTTCATCCAT | SEQ ID NO: 3609 | ACATGAACTCATTCCTTTTTATG | SEQ ID NO: 5975 |
| rs3869097 | CGCCACCCATTCTGGCTGT | SEQ ID NO: 1244 | CGCCACCCATTCTGGCTGC | SEQ ID NO: 3610 | CTTGAGGGGCCCTTCAG | SEQ ID NO: 5976 |
| rs2272593 | CCTGAGGGGCTTGGGGAA | SEQ ID NO: 1245 | CCTGAGGGGCTTGGGGAG | SEQ ID NO: 3611 | CTTGTGCCTTGGATGACC | SEQ ID NO: 5977 |
| rs3129792 | AGCCAGGCGTCACCTTCCACA | SEQ ID NO: 1246 | AGCCAGGCGTCACCTTCCACG | SEQ ID NO: 3612 | CGAACCTGCGAACCACAG | SEQ ID NO: 5978 |
| rs1264305 | CGCCCAAGAGCCAGCAAA | SEQ ID NO: 1247 | CGCCCAAGAGCCAGCAAG | SEQ ID NO: 3613 | GATCTCCACCTGCACAGG | SEQ ID NO: 5979 |
| rs4959047 | CCAAGAAGAGAAGGAAGGAATATATATTA | SEQ ID NO: 1248 | CCAAGAAGAGAAGGAAGGAATATATATTG | SEQ ID NO: 3614 | AACAAAAATAAACAGACCAAGAC | SEQ ID NO: 5980 |
| rs4713436 | CACTCTGTGTCAGGGGGTCAGAGA | SEQ ID NO: 1249 | CACTCTGTGTCAGGGGGTCAGAGG | SEQ ID NO: 3615 | GACCAGCACGGTTCTGGT | SEQ ID NO: 5981 |
| rs7829 | ACGCTGACCTGTGCTCCCTCT | SEQ ID NO: 1250 | ACGCTGACCTGTGCTCCCTCC | SEQ ID NO: 3616 | CAGAGAGGTGGGGCTGAG | SEQ ID NO: 5982 |
| rs3094696 | TCTTGGAGATGGGGAGTTTT | SEQ ID NO: 1251 | TCTTGGAGATGGGGAGAAG | SEQ ID NO: 3617 | AAGGAAGATAAATATTTAGCAAAAA | SEQ ID NO: 5983 |
| rs3828881 | AGCCTGAGGCCACAGTCCCAA | SEQ ID NO: 1252 | AGCCTGAGGCCACAGTCCCAT | SEQ ID NO: 3618 | CTGCCAGCCTGGAAGAACT | SEQ ID NO: 5984 |
| rs3094000 | GGGTGCTGGGGCAGAGAGA | SEQ ID NO: 1253 | GGGTGCTGGGGCAGAGAGG | SEQ ID NO: 3619 | GCAGGATCTGGATGGGAC | SEQ ID NO: 5985 |
| rs2855440 | ACTCCGCAGGCCCTCCAGTCT | SEQ ID NO: 1254 | ACTCCGCAGGCCCTCCAGTCC | SEQ ID NO: 3620 | AGGCTGCTGGCAGAGTCT | SEQ ID NO: 5986 |
| rs6457697 | AAATAGAGAATATAGGGAAAACACCTAGAA | SEQ ID NO: 1255 | AAATAGAGAATATAGGGAAAACACCTAGAC | SEQ ID NO: 3621 | ATAATAGTCAAAGTAAAAGCCACA | SEQ ID NO: 5987 |
| rs6919044 | GAATCCAAAATAAGTTTTTGTTTAAGAAA | SEQ ID NO: 1256 | GAATCCAAAATAAGTTTTTGAAAAGAAG | SEQ ID NO: 3622 | GAGAGAAAATTAATAATTGTTATGAA | SEQ ID NO: 5988 |
| rs1013518 | TGTATATTATCTGTGTTTACGTGGTAGAGA | SEQ ID NO: 1257 | TGTATATTATCTGTGTTTACGTGGTAGAGG | SEQ ID NO: 3623 | TTTATTACTTTATGGGTCACTGG | SEQ ID NO: 5989 |
| rs2144426 | AACTTACCAAAATTGACAGTAGAAGAAA | SEQ ID NO: 1258 | AACTTACCAAAATTGACAGTAGAAGAAG | SEQ ID NO: 3624 | ACAAAACAATAGTTTGATATAAT | SEQ ID NO: 5990 |
| rs4267952 | AACTTACCAAAATTGACAGTAGAAGAAA | SEQ ID NO: 1259 | AACTTACCAAAATTGACAGTAGAAGAAG | SEQ ID NO: 3625 | ACAATTTCAATAGTTTGATATTTAT | SEQ ID NO: 5991 |
| rs4947339 | AGATTCGAGGTTAAAACACAACTATACTA | SEQ ID NO: 1260 | AGATTCGAGGTTATTTACACAACTATACTG | SEQ ID NO: 3626 | AAGATAAAGTGAAAAATGTTAATTT | SEQ ID NO: 5992 |
| rs4618569 | CCCATGTTTGTATATATACCTGTGTGTAT | SEQ ID NO: 1261 | CCCATGTTTGTATATATACCTGTGTGTAC | SEQ ID NO: 3627 | TCTAGAAGGAGAGAATATATAATAGAA | SEQ ID NO: 5993 |
| rs7767288 | GGACACATATTTATCAGCCAACTATAAATA | SEQ ID NO: 1262 | GGACACATAAAATCAGCCAACTATAAATG | SEQ ID NO: 3628 | GCCATAGGATAGTGAAAGCAA | SEQ ID NO: 5994 |
| rs3752528 | TGGGCCCAGCCCCAGCAA | SEQ ID NO: 1263 | TGGGCCCAGCCCCAGCAG | SEQ ID NO: 3629 | GAGCCTGTAAAGATGGGAAC | SEQ ID NO: 5995 |
| rs3956986 | GGGCGCCCAGCCCAACCC | SEQ ID NO: 1264 | GGGCGCCCAGCCCAACCG | SEQ ID NO: 3630 | GCCCTGGGTTCTGTAAGC | SEQ ID NO: 5996 |
| rs2285320 | CTCACTGCTGGGCCGGTCAC | SEQ ID NO: 1265 | CTCACTGCTGGGCCGGTCAG | SEQ ID NO: 3631 | AGCCTCAGCTTCGCAAGT | SEQ ID NO: 5997 |
| rs6938690 | AATTAGCTAATCAGGTTTAGTTTTTTTCTA | SEQ ID NO: 1266 | AATTAGCTAATCAGGTTTAGTTTTTTTCTG | SEQ ID NO: 3632 | ATAGAAGTTTCATGTTCCTTGAAT | SEQ ID NO: 5998 |
| rs2532932 | CCAAGGTGTTGGGATTACAAGCATAAA | SEQ ID NO: 1267 | CCAAGGTGTTGGGATTACAAGCATAAG | SEQ ID NO: 3633 | ATTTTGGAGATGAAGACGTGT | SEQ ID NO: 5999 |
| rs1634725 | ACATATATGAAGAAAACTATGGACATTTTA | SEQ ID NO: 1268 | ACATATATGAAGAAAACTATGGACATTTTT | SEQ ID NO: 3634 | AAATGAGGCTATTATTAAATATCTAGAA | SEQ ID NO: 6000 |
| rs3117097 | AATCTCTGGAGTTTAACAAATGATTTT | SEQ ID NO: 1269 | AATCTCTGGAGTTTAACAAATGATTTC | SEQ ID NO: 3635 | ACATTTACATTGATCTATCTTTATATT | SEQ ID NO: 6001 |
| rs2621421 | CCTGAAGGAAGTGGTCACTAGACCAG | SEQ ID NO: 1270 | CCTGAAGGAAGTGGTCACTAGACCAC | SEQ ID NO: 3636 | ATGTAACCAAATACAGGATT | SEQ ID NO: 6002 |
| rs2076541 | AGTTAGTAAGAAAAATAAGATGAGGATGAA | SEQ ID NO: 1271 | AGTTAGTAAGAAAAATAAGATGAGGATGAG | SEQ ID NO: 3637 | ATAATATAGAATGAGGGATAAATGA | SEQ ID NO: 6003 |
| rs3094613 | GGCAAAAAAGTGAGACTCTGTTTCAAAT | SEQ ID NO: 1272 | GGCAAAAAAGTGAGACTCTGTTTCAAAA | SEQ ID NO: 3638 | ATAAAATAAAAAGGCCGGG | SEQ ID NO: 6004 |
| rs4711186 | CAGGTGAAATACAAAAAATTCATGAAAATT | SEQ ID NO: 1273 | CAGGTGAAATACAAAAAATTCATGAAAATG | SEQ ID NO: 3639 | CCTTTGGTACCAAACTCATACTA | SEQ ID NO: 6005 |
| rs3130165 | AGAAGCAATCGCACACCG | SEQ ID NO: 1274 | AGAAGCAATCGCACACCC | SEQ ID NO: 3640 | TGTCAGAAACTGGAGCCATAG | SEQ ID NO: 6006 |
| rs2395312 | GACCTTTATATCTTTCTAGAAACGTATCCT | SEQ ID NO: 1275 | GACCTTTATATCTTTCTAGAAACGTATCCG | SEQ ID NO: 3641 | CAAATTTATATGTATAGAGTTGGTCG | SEQ ID NO: 6007 |
| rs1627764 | AGGACCCGGGGAGCCGCT | SEQ ID NO: 1276 | AGGACCCGGGGAGCCGCG | SEQ ID NO: 3642 | AAGGAGGAGGGTCGGAC | SEQ ID NO: 6008 |
| rs204896 | GCGCTGCGTGGATGGCCA | SEQ ID NO: 1277 | GCGCTGCGTGGATGGCCG | SEQ ID NO: 3643 | CTTCACCGGTGAGGACTGT | SEQ ID NO: 6009 |
| rs7750714 | ACCGCCTTCGGGGCCTGA | SEQ ID NO: 1278 | ACCGCCTTCGGGGCCTGG | SEQ ID NO: 3644 | ACCCTGAGAGCCATGCCT | SEQ ID NO: 6010 |
| rs1054684 | TGGGTTATGTAAAGTGACAAATATTAAT | SEQ ID NO: 1279 | TGGGTTATGTAAAGTGACATTTATATTAAC | SEQ ID NO: 3645 | CAATAACAAACCCATTACATGA | SEQ ID NO: 6011 |
| rs7768931 | CCGAAGGCGGTGCATGGA | SEQ ID NO: 1280 | CCGAAGGCGGTGCATGGG | SEQ ID NO: 3646 | ATTGGGGATTCCCCATCT | SEQ ID NO: 6012 |
| rs2213573 | CAAATACTTGACTCAGTATTTTGAAGCTAA | SEQ ID NO: 1281 | CAAATACTTGACTCAGTATTTTGAAGCTAG | SEQ ID NO: 3647 | CTGGGTAAAGTATCTGATTGACA | SEQ ID NO: 6013 |
| rs241410 | TGGTAGGTAGAAATGAGGGACGTCATG | SEQ ID NO: 1282 | TGGTAGGTAGAAATGAGGGACGTCATC | SEQ ID NO: 3648 | ATCCCTGAATGGAATAAC | SEQ ID NO: 6014 |
| rs2523734 | ATGGATCACATATTGAAATGATAATAT | SEQ ID NO: 1283 | ATGGATCACATATTGAAATGATAATAG | SEQ ID NO: 3649 | TTGGAAATATAGGACTAAATAAAAAT | SEQ ID NO: 6015 |
| rs2163472 | TCTGCAGATAGTTTGAAAAAATATACATT | SEQ ID NO: 1284 | TCTGCAGATAGTTTGAAATTTATACATC | SEQ ID NO: 3650 | TATAACTGGTCTCA11ACACTCTGG | SEQ ID NO: 6016 |
| rs6930003 | CCCACCGAGCCCCGCCCT | SEQ ID NO: 1285 | CCCACCGAGCCCCGCCCC | SEQ ID NO: 3651 | GATTTTGGCTGAAAATGAAAC | SEQ ID NO: 6017 |
| rs4713193 | AACAAAACAAACAAACAAACAAACAAAA | SEQ ID NO: 1286 | AACAAAACAAACAAACAAACAAACAAAC | SEQ ID NO: 3652 | AAACATAGCATCACTTTTACCAG | SEQ ID NO: 6018 |
| rs1264321 | CTACTTACCAGCCCCCCTACACAT | SEQ ID NO: 1287 | CTACTTACCAGCCCCCCTACACAC | SEQ ID NO: 3653 | CACACTGACACACACACACA | SEQ ID NO: 6019 |
| rs204478 | AAATAACTTTTACTATAAGGGGAAAGATTT | SEQ ID NO: 1288 | AAATAACTTTTACTATAAGGGGAAAGATTA | SEQ ID NO: 3654 | AGATAGTTTTCCAGAACATGGTT | SEQ ID NO: 6020 |
| rs2301752 | CGCCGCCTGCTCATCTCCA | SEQ ID NO: 1289 | CGCCGCCTGCTCATCTCCG | SEQ ID NO: 3655 | GTACAACTGGCCCCACC | SEQ ID NO: 6021 |
| rs6901830 | CTTCCATTTTTGTCCCCTGGACA | SEQ ID NO: 1290 | CTTCCATTTTTGTCCCCTGGACG | SEQ ID NO: 3656 | ATGCTGTAGGTGTCAGC | SEQ ID NO: 6022 |
| rs3998159 | GACATGTAGGCATTATCTAATGGTCAAAT | SEQ ID NO: 1291 | GACATGTAGGCATTATCTAATGGTCAAAG | SEQ ID NO: 3657 | TACTTACAATTGTACATAAAAATAA | SEQ ID NO: 6023 |
| rs2246986 | AGGGAACAAATAATAAAGAAACAAGATA | SEQ ID NO: 1292 | AGGGAACAAATAATAAAGAAACAAGATG | SEQ ID NO: 3658 | AATTAGATGTAAAATAGATGAGAAAAA | SEQ ID NO: 6024 |
| rs3117113 | AGATCCTTAATAAGTACCTCAAAATGAAT | SEQ ID NO: 1293 | AGATCCTTAATAAGTACCTCTTAAATGAAG | SEQ ID NO: 3659 | TCCCTAATTTACAGAAGAGAAACT | SEQ ID NO: 6025 |
| rs2857128 | GACTACATCAGTAGCTTATAATTCTTTTGA | SEQ ID NO: 1294 | GACTACATCAGTAGCTTATAATTCTTTTGG | SEQ ID NO: 3660 | ATCAGGATCCCTTTTTATTC | SEQ ID NO: 6026 |
| rs2516643 | TGCCATGTTGCCTGAGATT | SEQ ID NO: 1295 | TGCCATGTTGCCTGAGATC | SEQ ID NO: 3661 | CTTGGCCTCAAAGTGCTG | SEQ ID NO: 6027 |
| rs3117103 | TTAAATGATTGAAGTATAATTTACAGACAA | SEQ ID NO: 1296 | TTAAATGATTGAAGTATAATTTACAGACAT | SEQ ID NO: 3662 | TATTTTTACAAGCACAGATTATAAGT | SEQ ID NO: 6028 |
| rs2233965 | GGTAAAAGTCACTGCAGCA | SEQ ID NO: 1297 | GGTAATTTGTCACTGCAGCC | SEQ ID NO: 3663 | CAGGAATTAGTCAGTATCTCATAAG | SEQ ID NO: 6029 |
| rs6916394 | ATGCCACACATAATTTATCGT | SEQ ID NO: 1298 | ATGCCACACATAATTTATCGC | SEQ ID NO: 3664 | TTAAAGTGTACAGATCAGTGGCT | SEQ ID NO: 6030 |
| rs2074485 | CCAGGGCCCCCAAGAGCA | SEQ ID NO: 1299 | CCAGGGCCCCCAAGAGCG | SEQ ID NO: 3665 | CAGGAGGAGGGTTCGG | SEQ ID NO: 6031 |
| rs1619893 | GTGAGACTCCATCTCAAAAAAAAAAAA | SEQ ID NO: 1300 | GTGAGACTCCATCTCAAAAAAAAAAAG | SEQ ID NO: 3666 | AAAAGAAATAGTCAGTTGAAACATT | SEQ ID NO: 6032 |
| rs4449615 | TCTGAATIATTTCAGATTACATACATAAAA | SEQ ID NO: 1301 | TCTGAATTATTTCAGATTACATACATTAAG | SEQ ID NO: 3667 | ATGACTTCATTAATAGGTAATATCACA | SEQ ID NO: 6033 |
| rs3115611 | TGGTCCCTCCCCCAATAGGTAGG | SEQ ID NO: 1302 | TGGTCCCTCCCCCAATAGGTAGC | SEQ ID NO: 3668 | ATTACAATTCGCATAACAATTCA | SEQ ID NO: 6034 |
| rs2301748 | CCCATGGCCCCGACGTCA | SEQ ID NO: 1303 | CCCATGGGCCCGACGTCG | SEQ ID NO: 3669 | CACCCTCTCAGCGGCTC | SEQ ID NO: 6035 |
| rs3129845 | GGATCTGTATTAGGTGCTCCAGAGCA | SEQ ID NO: 1304 | GGATCTGTATTAGGTGCTCCAGAGCG | SEQ ID NO: 3670 | TTGTACTAACAGGATATGTATATATAG | SEQ ID NO: 6036 |
| rs3130829 | TTTAACTCTAATAGTCTCCTTTGATTATGT | SEQ ID NO: 1305 | TTTAACTCTAATAGTCTCCTTTGATTATGC | SEQ ID NO: 3671 | AACTAAATATTGTCATTAATTTAAGGA | SEQ ID NO: 6037 |
| rs6457125 | TTGTCTGGGATGTTGCTCCTCAT | SEQ ID NO: 1306 | TTGTCTGGGATGTTGCTCCTCAC | SEQ ID NO: 3672 | TGTAAGATGTAAATGTGGATATGG | SEQ ID NO: 6038 |
| rs2571402 | ATCGGGTCCAAATGCAAGACCT | SEQ ID NO: 1307 | ATCGGGTCCAAATGCAAGACCA | SEQ ID NO: 3673 | AGGGAGCTAATAATTAGAGGGAT | SEQ ID NO: 6039 |
| rs2157605 | GAAAACACCCCCACTGTGTTGAGT | SEQ ID NO: 1308 | GATAACACCCCCACTGTGTTGAGC | SEQ ID NO: 3674 | GCAACAAAGTGGATATTAAGGAC | SEQ ID NO: 6040 |
| rs2275853 | AGCCACATCTCCTGTGGGATCTC | SEQ ID NO: 1309 | AGCCACATCTCCTGTGGGATCTG | SEQ ID NO: 3675 | CCAGGTTCTGTTTTTGTTCTAC | SEQ ID NO: 6041 |
| rs3132710 | GGCTGAATTGCTGGATGATTCTGT | SEQ ID NO: 1310 | GGCTGAATTGCTGGATGATTCTGC | SEQ ID NO: 3676 | CACACTTACAATGAGGTAAGGG | SEQ ID NO: 6042 |
| rs2654005 | AGATGCAGTGAAGGCACAACCTACT | SEQ ID NO: 1311 | AGATGCAGTGAAGGCACAACCTACC | SEQ ID NO: 3677 | CTACAGGCCCTTAGGAATTATTA | SEQ ID NO: 6043 |
| rs1011985 | TGTCCACTTGTGCCTCCCACTTC | SEQ ID NO: 1312 | TGTCCACTTGTGCCTCCCACTTG | SEQ ID NO: 3678 | TGGTAGTTATTCTTTTGTATGCAC | SEQ ID NO: 6044 |
| rs2075789 | GCTTCCCCAGCCCGGCCT | SEQ ID NO: 1313 | GCTTCCCCAGCCCGGCCC | SEQ ID NO: 3679 | CGAGGAGGAGGAAGTCGAG | SEQ ID NO: 6045 |
| rs2076182 | CAGAGCGAGGCTGGTGAGTGAA | SEQ ID NO: 1314 | CAGAGCGAGGCTGGTGAGTGAC | SEQ ID NO: 3680 | CAGGTCACGACCACCC | SEQ ID NO: 6046 |
| rs3132684 | CCCCATCTCTACCAAAAAACAGAAA | SEQ ID NO: 1315 | CCCCATCTCTACCAAAAAACAGAAG | SEQ ID NO: 3681 | GTGGGCTTGCACCTGTAGT | SEQ ID NO: 6047 |
| rs238877 | TTGTAGTAGGCAAGAAAAAGAAATTAAATGA | SEQ ID NO: 1316 | TTGTAGTAGGCAAGAAAAAGAAATAAATGG | SEQ ID NO: 3682 | AATACAAATGTACTTATTTTCAGATG | SEQ ID NO: 6048 |
| rs2072681 | GGGAGGGAAAAGGGTCCTATAGAAT | SEQ ID NO: 1317 | GGGAGGGAAAAGGGTCCTATAGAAC | SEQ ID NO: 3683 | AGGGAAGACAAAAATGTTTTATT | SEQ ID NO: 6049 |
| rs6919908 | GCCTGAACCTGAATCCCCTGTCA | SEQ ID NO: 1318 | GCCTGAACCTGAATCCCCTGTCG | SEQ ID NO: 3684 | AGATGTATGGGACACATCTCTT | SEQ ID NO: 6050 |
| rs2647045 | AGAATGCTGCAGATGGTGGCA | SEQ ID NO: 1319 | AGAATGCTGCAGATGGTGGCG | SEQ ID NO: 3685 | AATGTTCAATGTGAAAAAGGATA | SEQ ID NO: 6051 |
| rs1632986 | GGACACTGCATTCATTTCTTTTGGT | SEQ ID NO: 1320 | GGACACTGCATTCATTTCTTTTGGC | SEQ ID NO: 3686 | CACTTATTTCTTTTGGAATGGA | SEQ ID NO: 6052 |
| rs2395470 | CTTCTGCCTCCCTCATCCCTCA | SEQ ID NO: 1321 | CTTCTGCCTCCCTCATCCCTCG | SEQ ID NO: 3687 | CTCTCAATTCTGTTCTCAGCAC | SEQ ID NO: 6053 |
| rs915668 | AGCCCATCACATGCTCAATACATTC | SEQ ID NO: 1322 | AGCCCATCACATGCTCAATACATTG | SEQ ID NO: 3688 | AGTCAAAGAAACCTCATAGCAC | SEQ ID NO: 6054 |
| rs2295119 | GAGTTTGTGCAGACGCACAGAA | SEQ ID NO: 1323 | GAGTTTGTGCAGACGCACAGAC | SEQ ID NO: 3689 | CTCTGGGGAGTATATGTTTGAAT | SEQ ID NO: 6055 |
| rs2071536 | GGCGCGGAGAGTCCCAGA | SEQ ID NO: 1324 | GGCGCGGAGAGTCCCAGG | SEQ ID NO: 3690 | GTCCGAGTGCCAATGGCTA | SEQ ID NO: 6056 |
| rs3130827 | TCTTGTGTCATATGGCTTCATTGCTA | SEQ ID NO: 1325 | TCTTGTGTCATATGGCTTCATTGCTG | SEQ ID NO: 3691 | GGCTGTACTCAAGATCAAGTCT | SEQ ID NO: 6057 |
| rs3093556 | GTGGTGCGAGTGCGTGAATAT | SEQ ID NO: 1326 | GTGGTGCGAGTGCGTGAATAC | SEQ ID NO: 3692 | AGGACCCCATGGCAGT | SEQ ID NO: 6058 |
| rs3734818 | CGGGAGTCACAGATACATTGTCTGGA | SEQ ID NO: 1327 | CGGGAGTCACAGATACATTGTCTGGG | SEQ ID NO: 3693 | AAGTGAAACTTAGGAGCTTTGTG | SEQ ID NO: 6059 |
| rs4713270 | AGAAGCTGTAGGGAACAGGCTTGTA | SEQ ID NO: 1328 | AGAAGCTGTAGGGAACAGGCTTGTG | SEQ ID NO: 3694 | ATAAGGCTGCTAAGCAAGTCTC | SEQ ID NO: 6060 |
| rs1988565 | GAATGCTGCAGATGGTGGCAGA | SEQ ID NO: 1329 | GAATGCTGCAGATGGTGGCAGG | SEQ ID NO: 3695 | TGCTCAATGTGAAAAAGGATAG | SEQ ID NO: 6061 |
| rs1233333 | CTCCTCCGCGCAGGGGCT | SEQ ID NO: 1330 | CTCCTCCGCGCAGGGGCC | SEQ ID NO: 3696 | TTTCCCTCCTGACCCC | SEQ ID NO: 6062 |
| rs2074489 | GGACCTGGGACTTCGTCCTGATT | SEQ ID NO: 1331 | GGACCTGGGACTTCGTCCTGATC | SEQ ID NO: 3697 | CTCTTCTCCTACACCAAGCAT | SEQ ID NO: 6063 |
| rs1736981 | GGGCTGTTCAGTGTCACAACAAACAA | SEQ ID NO: 1332 | GGGCTGTTCAGTGTCACAACTTACTG | SEQ ID NO: 3698 | GACTGACCTGGATTAGTTTATGA | SEQ ID NO: 6064 |
| rs3129005 | AAAGATGGAGACATCTCAGCCCCT | SEQ ID NO: 1333 | AAAGATGGAGACATCTCAGCCCCG | SEQ ID NO: 3699 | CTCTCTGGAACAGGTAAGATG | SEQ ID NO: 6065 |
| rs3998357 | GGTGAGGACAAGGAGCAGGAGAAA | SEQ ID NO: 1334 | GGrGAGGACAAGGAGCAGGAGAAG | SEQ ID NO: 3700 | GACTCTAAAGCAGTGGAGGAG | SEQ ID NO: 6066 |
| rs3132645 | CATAGTAGATTCCTGATGGGTTTTCTCT | SEQ ID NO: 1335 | CATAGTAGATTCCTGATGGGTTTTCTCC | SEQ ID NO: 3701 | ATGATTAAATTATATATTGTCTATGT | SEQ ID NO: 6067 |
| rs1264548 | AGAAAATATATTCTGTATGATTTCAATGTT | SEQ ID NO: 1336 | AGAAAATATATTCTGTATGATTTCAATGTC | SEQ ID NO: 3702 | CTGAAATATGGTTTCTCCTGGTA | SEQ ID NO: 6068 |
| rs2428495 | GGGAAAGGCCTGGGTAATGGA | SEQ ID NO: 1337 | GGGAAAGGCCTGGGTAATGGG | SEQ ID NO: 3703 | ATTCTTTGATTGGGATGTTTC | SEQ ID NO: 6069 |
| rs3817824 | TGGGCAGAGGGGACACAACTAA | SEQ ID NO: 1338 | TGGGCAGAGGGGACACAACTAG | SEQ ID NO: 3704 | CTCTGGAGATTCTTTGATTTGG | SEQ ID NO: 6070 |
| rs1632948 | TCACAGAAAATTATAAAATGTAAAACACTA | SEQ ID NO: 1339 | TCACAGAAAATTATAAAATGTAAAACACTG | SEQ ID NO: 3705 | TTTATGTAACTAGTGTGCGTATCA | SEQ ID NO: 6071 |
| rs1736998 | ACCCCAATAACCACCCTTGGTTA | SEQ ID NO: 1340 | ACCCCAATAACCACCCTTGGTTT | SEQ ID NO: 3706 | TCAGGTTACCCCAGAACTCA | SEQ ID NO: 6072 |
| rs2535252 | GTCCCTTGAGGGACTTCAGATG | SEQ ID NO: 1341 | GTCCCTTGAGGGACTTCAGATC | SEQ ID NO: 3707 | AAGTCCCTTAGCTGCTC | SEQ ID NO: 6073 |
| rs1625485 | CGCAGGACCCGGGGAGCA | SEQ ID NO: 1342 | CGCAGGACCCGGGGAGCC | SEQ ID NO: 3708 | CGCAAGGAGGAGGGTC | SEQ ID NO: 6074 |
| rs1811197 | CTGGTAGGAATGCAAAAAATGTGGT | SEQ ID NO: 1343 | CTGGTAGGAATGCAAAAAATGTGGC | SEQ ID NO: 3709 | GCCAATTTGTAAAGTGGTATG | SEQ ID NO: 6075 |
| rs2524173 | CTGGTCCCAGGATTGATAGCAACA | SEQ ID NO: 1344 | CTGGTCCCAGGATTGATAGCAACG | SEQ ID NO: 3710 | GCCTATAACTGGTCTCCTGGT | SEQ ID NO: 6076 |
| rs6925580 | CTGCCATAGCTGGAGAGCCCTT | SEQ ID NO: 1345 | CTGCCATAGCTGGAGAGCCCTG | SEQ ID NO: 3711 | GGACCTTGCACTTTTCCTATT | SEQ ID NO: 6077 |
| rs3828903 | CACAATCTATTGTCTGGCACCACCT | SEQ ID NO: 1346 | CACAATCTATTGTCTGGCACCACCC | SEQ ID NO: 3712 | CATTTCTCTGGTGACATTTCC | SEQ ID NO: 6078 |
| rs2596450 | GAGTGGAAAGATGGTCAGAGAATGGA | SEQ ID NO: 1347 | GAGTGGAAAGATGGTCAGAGAATGGG | SEQ ID NO: 3713 | AGTGTGAGATAGAGAGTATGGAAG | SEQ ID NO: 6079 |
| rs6457042 | TCCCCACTGTCTACTCTGATAGCCT | SEQ ID NO: 1348 | TCCCCACTGTCTACTCTGATAGCCC | SEQ ID NO: 3714 | AAGGAAACAGGGACCTTAATC | SEQ ID NO: 6080 |
| rs1799908 | GGTCCCTCTGGACCTCAGGGT | SEQ ID NO: 1349 | GGTCCCTCTGGACCTCAGGGA | SEQ ID NO: 3715 | CTCTAGGATACCCAGGACCTC | SEQ ID NO: 6081 |
| rs8365 | GGAACTTTTGCCCACCTTTGAAGATG | SEQ ID NO: 1350 | GGAACTTTGCCCACCTTTGAAGATC | SEQ ID NO: 3716 | AATCAAGAAATCAGGGAAAGC | SEQ ID NO: 6082 |
| rs3823340 | GCACTGTCACTGCCTGGGGTAGA | SEQ ID NO: 1351 | GCACTGTCACTGCCTGGGGTAGG | SEQ ID NO: 3717 | CAAAAACAGAACCTGGTCAGAT | SEQ ID NO: 6083 |
| rs1611589 | AATTGCATTTGCAAAAATGTATCATCA | SEQ ID NO: 1352 | AATTGCATTTGCTTTAAATGTATCATCG | SEQ ID NO: 3718 | CATCATTATTATTTTTGAGACA | SEQ ID NO: 6084 |
| rs2071291 | GGGTATCCGGGCGCTACGTAA | SEQ ID NO: 1353 | GGGTATCCGGGCGCTACGTAG | SEQ ID NO: 3719 | CAGCGTGCCCACAGTC | SEQ ID NO: 6085 |
| rs1233387 | AGTGGAGCTTGGGGTCCAGCA | SEQ ID NO: 1354 | AGTGGAGCTTGGGGTCCAGCG | SEQ ID NO: 3720 | AGACAGCAGGATGATGAGTGT | SEQ ID NO: 6086 |
| rs6920823 | GCTCCCCTTTGCCTAAACCCTATT | SEQ ID NO: 1355 | GCTCCCCTTTGCCTAAACCCTATG | SEQ ID NO: 3721 | CCTCCTGTGCATCTGTACTC | SEQ ID NO: 6087 |
| rs2251731 | TGAAGAAATGAGGGAAGAGATAGGAAT | SEQ ID NO: 1356 | TGAAGAAATGAGGGAAGAGATAGGAAG | SEQ ID NO: 3722 | AAAGGGGAGAGAAAAAGTAAAG | SEQ ID NO: 6088 |
| rs6920830 | CCCCACAGCTTTGATGGCAGT | SEQ ID NO: 1357 | CCCCACAGCTTTGATGGCAGC | SEQ ID NO: 3723 | CCTCATCTTCAACTTTTGTGC | SEQ ID NO: 6089 |
| rs3873375 | TCTCCCTGAATCCTCAACGCTAATA | SEQ ID NO: 1358 | TCTCCCTGAATCCTCAACGCTAATG | SEQ ID NO: 3724 | GGACTGTCTCCTTCTCACCTC | SEQ ID NO: 6090 |
| rs1737004 | CACCACATCCTGAAAGATGGCAC | SEQ ID NO: 1359 | CACCACATCCTGAAAGATGGCAG | SEQ ID NO: 3725 | CATTCCTCAGAGGGTTTCAT | SEQ ID NO: 6091 |
| rs6457611 | TTCTTTGTGTTAGCAAACTTATTTATAGAT | SEQ ID NO: 1360 | TTCTTTGTGTTAGCAAACTTATTTATAGAC | SEQ ID NO: 3726 | ATTATCCATTAAAGATTATACTATCCA | SEQ ID NO: 6092 |
| rs2524088 | AATGGTAGACCAAAATCCAACCATTA | SEQ ID NO: 1361 | AATGGTAGACCAAAATCCAACCATTG | SEQ ID NO: 3727 | AAGTGAAGAAATGACACGACC | SEQ ID NO: 6093 |
| rs3749962 | CAGCCTGTCCTGGACAGTTCCT | SEQ ID NO: 1362 | CAGCCTGTCCTGGACAGTTTCCC | SEQ ID NO: 3728 | AGGGACAGTTTGACCACTTC | SEQ ID NO: 6094 |
| rs1737085 | ACCCAGGGGCCTCAGAAAGACT | SEQ ID NO: 1363 | ACCCAGGGGCCTCAGAAAGACG | SEQ ID NO: 3729 | GTTTGTCCGAAAAGTGAGAG | SEQ ID NO: 6095 |
| rs7382817 | TTCCACTTGGTCATGAAGAATGAA | SEQ ID NO: 1364 | TTCCACTTGGTCATGAAGAATGAT | SEQ ID NO: 3730 | TTTTTAATATACTGTTGAATGTGGT | SEQ ID NO: 6096 |
| rs2394413 | GCTTTCCTGTTTTTGTTTTTCTTCTTCTTT | SEQ ID NO: 1365 | GCTTTCCTGTTTTTGTTTTTCTTCTTCTTC | SEQ ID NO: 3731 | CCCTCCCCTTGTTTGTTTTTA | SEQ ID NO: 6097 |
| rs2073151 | CATGCCCTGCTGCACTCCA | SEQ ID NO: 1366 | CATGCCCTGCTGCACTCCG | SEQ ID NO: 3732 | AATGACTTCTCGCAGAACTT | SEQ ID NO: 6098 |
| rs1058026 | TTCTCAGGTCTTTATTTGCTCTCTCAA | SEQ ID NO: 1367 | TTCTCAGGTCTTTATTTGCTCTCTCAC | SEQ ID NO: 3733 | TTCCAGGAATTGACTTATTTAATT | SEQ ID NO: 6099 |
| rs2272873 | AGCCTCAACACATCCTCTTCCCTT | SEQ ID NO: 1368 | AGCCTCAACACATCCTCTTCCCTC | SEQ ID NO: 3734 | GGTTTTCTGATCCTGACCTG | SEQ ID NO: 6100 |
| rs1632933 | TTGTAGTGATGGGGACCTGATCCT | SEQ ID NO: 1369 | TTGTAGTGATGGGGACCTGATCCC | SEQ ID NO: 3735 | GTAATCACAGGTCAGGGGAAG | SEQ ID NO: 6101 |
| rs1632939 | GACTCAGAGGAGCTGGAGTCAGACT | SEQ ID NO: 1370 | GACTCAGAGGAGCTGGAGTCAGACC | SEQ ID NO: 3736 | CCACACACATTGAGTGTGAG | SEQ ID NO: 6102 |
| rs3132704 | TGCTATTCCATCACTGGGTATTCCTT | SEQ ID NO: 1371 | TGCTATTCCATCACTGGGTATTCCTC | SEQ ID NO: 3737 | CTACTCTTTTCATGTTTCATTTATTT | SEQ ID NO: 6103 |
| rs2523611 | GCCTCTGTGGTCACAGCTCCCA | SEQ ID NO: 1372 | GCCTCTGTGGTCACAGCTCCCG | SEQ ID NO: 3738 | TGGACAAGTTTCCACTGAAG | SEQ ID NO: 6104 |
| rs3823313 | TCTACAATGGCATTCAATAAAGTGCAT | SEQ ID NO: 1373 | TTTACAATGGCATTCAATAAAGTGCAG | SEQ ID NO: 3739 | TGTTCCTGGTTAAAAAAAAAA | SEQ ID NO: 6105 |
| rs2524083 | TCCTTTCTGTATATCTGGGTCACCATT | SEQ ID NO: 1374 | TCCTTTCTGTATATCTGGGTCACCATC | SEQ ID NO: 3740 | AGTATCATTTCCCTTCAGCCT | SEQ ID NO: 6106 |
| rs983561 | CCACTCCGAAGTCAGCACAGAGA | SEQ ID NO: 1375 | CCACTCCGAAGTCAGCACAGAGC | SEQ ID NO: 3741 | TTAGGATATAGGACATTCTGTTGC | SEQ ID NO: 6107 |
| rs915660 | CCCAAAATGCATGAAATTCCCTTC | SEQ ID NO: 1376 | CCCAAAATGCATGAAATTCCCTTG | SEQ ID NO: 3742 | TCTGACTGATAAACCCTCACTC | SEQ ID NO: 6108 |
| rs1269839 | CCCCTCTCCCCACTTATTCTCCTA | SEQ ID NO: 1377 | CCCCTCTCCCCACTTATTCTCCTC | SEQ ID NO: 3743 | TTGGAATCTTCAACTTCTGAAG | SEQ ID NO: 6109 |
| rs2050190 | TCTGAAATGCCCTGTCTATAGGGCA | SEQ ID NO: 1378 | TCTGAAATGCCCTGTCTATAGGGCG | SEQ ID NO: 3744 | TTAGAAGATGGTCCACCCTATTA | SEQ ID NO: 6110 |
| rs8408 | CAGTCACCCCCACTTCCCACT | SEQ ID NO: 1379 | CAGTCACCCCCACTTCCCACC | SEQ ID NO: 3745 | GCAGTCTTGTAGCTAGAACTTCTC | SEQ ID NO: 6111 |
| rs3115573 | CCCCCCACTTCTTGAAAAGACTTATT | SEQ ID NO: 1380 | CCCCCCACTTCTTGAAAAGACTTATC | SEQ ID NO: 3746 | TTTTAATCTTTCTAACCACAGCA | SEQ ID NO: 6112 |
| rs2230365 | CTGCTAAGCCGGTGTCCCTCT | SEQ ID NO: 1381 | CTGCTAAGCCGCTGTCCCTCC | SEQ ID NO: 3747 | CCATGGGAATAAAGAATAAGGAT | SEQ ID NO: 6113 |
| rs2284178 | CACCAACCAAGGGCTGCTGA | SEQ ID NO: 1382 | CACCAACCAAGGGGTGCTGG | SEQ ID NO: 3748 | CATAAAGAATGTTGAAAAGAGCTT | SEQ ID NO: 6114 |
| rs720467 | GAGGCTTCACCCAGTTAGCCCA | SEQ ID NO: 1383 | GAGGCTTCACCCAGTTAGCCCG | SEQ ID NO: 3749 | GAGCTCTAAGGCTGTTCTGATC | SEQ ID NO: 6115 |
| rs1842399 | TCCTCCACTAGCATTATCCCCAGG | SEQ ID NO: 1384 | TCCTCCACTAGCATTATCCCCAGC | SEQ ID NO: 3750 | CAATGACTTTTAATGGTAATATGTG | SEQ ID NO: 6116 |
| rs2857106 | CAATGGAAAGTGAACAAAAGGTTCCT | SEQ ID NO: 1385 | CAATGGAAAGTGAACAAAAGGTTCCC | SEQ ID NO: 3751 | GTCAAAGAATTTAACTCCTCGTG | SEQ ID NO: 6117 |
| rs1894552 | TGGGGTTAACTCCCAATAAATCCAT | SEQ ID NO: 1386 | TGGGGAAACTCCCAATAAATCCAG | SEQ ID NO: 3752 | GTAAATTGAAAATATTGTGAGTTGA | SEQ ID NO: 6118 |
| rs1048664 | TGCTGAATTGTCACTCATGGGCT | SEQ ID NO: 1387 | TGCTGAATTGTCACTCATGGGCC | SEQ ID NO: 3753 | GAGAGTAGGAGACTGGAGACC | SEQ ID NO: 6119 |
| rs2076529 | GTCAATGGCGTCACTCACCAGT | SEQ ID NO: 1388 | GTCAATGGCGTCACTCACCAGC | SEQ ID NO: 3754 | CAGTCCTCCCTCTGTACTCTG | SEQ ID NO: 6120 |
| rs2269422 | TCTTCTCCAAGGCCTCCCTCTTA | SEQ ID NO: 1389 | TCTTCTCCAAGGCCTCCCTCTTG | SEQ ID NO: 3755 | CTAAGAAAAAGCCTTCAACCC | SEQ ID NO: 6121 |
| rs3135338 | CTTTTCTCTGGACATATGCAGCATTA | SEQ ID NO: 1390 | CTTTTCTCTGGACATATGCAGCATTG | SEQ ID NO: 3756 | TTGGAAATCTTTTTTCATTAAGC | SEQ ID NO: 6122 |
| rs3129907 | GGTCAAATAAAAGAAGGAATGGCAAA | SEQ ID NO: 1391 | GGTCAAATAAAAGAAGGAATGGCAAG | SEQ ID NO: 3757 | TATTTACTCAGCTGACTGAGAATT | SEQ ID NO: 6123 |
| rs2621413 | TCAGAGTATCCTTTCTCCTTTGCAAT | SEQ ID NO: 1392 | TCAGAGTATCCTTTCTCCTTTGCAAG | SEQ ID NO: 3758 | AATTGCTCTATGTTGTATCTCCTT | SEQ ID NO: 6124 |
| rs3129848 | TGTGTTTGTGACCAGAAATAAGCCAT | SEQ ID NO: 1393 | TGTGTTTGTGACCAGAAATAAGCCAC | SEQ ID NO: 3759 | GGAACTTAAATCTTGTTCGCAT | SEQ ID NO: 6125 |
| rs2395253 | TCCTCTCCCCTTCTCACCTTGAA | SEQ ID NO: 1394 | TCCTCTCCCCTTCTCACCTTGAG | SEQ ID NO: 3760 | AGATAAAAATTCTCTAGGCAGGA | SEQ ID NO: 6126 |
| rs3135365 | TTTTATCATTGTTACCAGCAAGAGGGT | SEQ ID NO: 1395 | TTTATCATTGTTACCAGCAAGAGGGG | SEQ ID NO: 3761 | ATTTCTATACAAACTACTCCGTC | SEQ ID NO: 6127 |
| rs2395106 | AGGATCTCATTTGGCATCCTGCA | SEQ ID NO: 1396 | AGGATCTCATTTGGCATCCTGCG | SEQ ID NO: 3762 | TGCATTTAGCTGTCGAGTCTT | SEQ ID NO: 6128 |
| rs2285796 | CAGTAGGGAGATGACAGAGCATTGTAT | SEQ ID NO: 1397 | CAGTAGGGAGATGACAGAGCATTGTAG | SEQ ID NO: 3763 | TTACACTGAGCAGAGTAAACAGAT | SEQ ID NO: 6129 |
| rs2284177 | ATGTGGCCAGTGGCTACCGTATC | SEQ ID NO: 1398 | ATGTGGCCAGTGGCTACCGTATG | SEQ ID NO: 3764 | GACAGCACAGACAGGAAACAT | SEQ ID NO: 6130 |
| rs3115563 | CACAAGTATTTTGCAGCAGCTATCATT | SEQ ID NO: 1399 | CACAAGTATTTTGCAGCAGCTATCATA | SEQ ID NO: 3765 | AAAATGTTCCAACAAGCAATTAT | SEQ ID NO: 6131 |
| rs2074477 | GAGAGCTGGAGCTGAAGTTCCCT | SEQ ID NO: 1400 | GAGAGCTGGAGCTGAAGTTCCCC | SEQ ID NO: 3766 | CAGACCAGGAACTACCACAC | SEQ ID NO: 6132 |
| rs2301755 | AACAGACAGGGACATCAGGCAAT | SEQ ID NO: 1401 | AACAGACAGGGACATCAGGCAAC | SEQ ID NO: 3767 | TAGACCCTCCTCCCTTCCTAT | SEQ ID NO: 6133 |
| rs2057727 | ACCGTTGTGCCACTGGACGAT | SEQ ID NO: 1402 | ACCGTTGTGCCACTGGACGAC | SEQ ID NO: 3768 | CTACACAGGAGTACAAGGTGG | SEQ ID NO: 6134 |
| rs2269705 | AACCCCCCTTCTGAGACTTGGAT | SEQ ID NO: 1403 | AACCCCCCTTCTGAGACTTGGAC | SEQ ID NO: 3769 | CCTGATCCTGACTTCTGATC | SEQ ID NO: 6135 |
| rs2076177 | GGGAGATGGGTAAAGAGGGGAAT | SEQ ID NO: 1404 | GGGAGATGGGTAAAGAGGGGAAC | SEQ ID NO: 3770 | AGGGGTCATGTCTTTTCTCAG | SEQ ID NO: 6136 |
| rs2071285 | GTGGGAGGGAGCGTGAGGCT | SEQ ID NO: 1405 | GTGGGAGGGAGCGTGAGGCA | SEQ ID NO: 3771 | GACATAGCATCAGATTCTCAGC | SEQ ID NO: 6137 |
| rs1109146 | GGCTCTCCATGGGTTCTTAGAGATT | SEQ ID NO: 1406 | GGCTCTCCATGGGTTCTTAGAGATC | SEQ ID NO: 3772 | TTTGCAAGGGATATGAAGGATA | SEQ ID NO: 6136 |
| rs2395178 | AGGGAACTGAAAGTCATTTCTCAAGTC | SEQ ID NO: 1407 | AGGGAACTGAAAGTCATTTCTCAAGTG | SEQ ID NO: 3773 | CCCAAATTTAGTAATCTCACAAG | SEQ ID NO: 6139 |
| rs1978029 | GCTCTTCTCACCAAAACTTAAGGCTCT | SEQ ID NO: 1408 | GCTCTTCTCACCAAAACTTAAGGCTCC | SEQ ID NO: 3774 | CAGAGTGTCTAATAGAAGTGAACAT | SEQ ID NO: 6140 |
| rs406359 | ACAGCCTAAGGAGGGAAACAAAAAT | SEQ ID NO: 1409 | ACAGCCTAAGGAGGGAAACAAAAAC | SEQ ID NO: 3775 | CCAGTCAACACATAGTGTGTTG | SEQ ID NO: 6141 |
| rs2239529 | GGTCATGAGGGAACGGAAGCA | SEQ ID NO: 1410 | GGTCATGAGGGAACGGAAGCG | SEQ ID NO: 3776 | GGAAACACTATGTTGCCTCC | SEQ ID NO: 6142 |
| rs2269425 | TCAAGTAGTCCGTGTCTGGCAAGT | SEQ ID NO: 1411 | TCAAGTAGTCCGTGTCTGGCAAGC | SEQ ID NO: 3777 | AATGGCAATGCTAAGTGACC | SEQ ID NO: 6143 |
| rs2073048 | ATTAGAAAGGTTAAGGAACCGCTCT | SEQ ID NO: 1412 | ATTAGAAAGGTTAAGGAACCGCTCC | SEQ ID NO: 3778 | AAACAATTTCCTGGATGTTTAAA | SEQ ID NO: 6144 |
| rs3806155 | AACAACCCAGGTATGCTGGTATTAATAA | SEQ ID NO: 1413 | AACAACCCAGGTATGCTGGTATTAATAT | SEQ ID NO: 3779 | TTGAAGATGAAGAAATTAGGACTT | SEQ ID NO: 6145 |
| rs436845 | TCACTGTGGGTGGAGGTAATGGTAT | SEQ ID NO: 1414 | TCACTGTGGGTGGAGGTAATGGTAC | SEQ ID NO: 3780 | CTCCTCCTGGGGGTATTTTA | SEQ ID NO: 6146 |
| rs434841 | CAGCCCCAGTGCGCTCCA | SEQ ID NO: 1415 | CAGCCCCAGTGCCCTCCG | SEQ ID NO: 3781 | CCCAGTTACTAATCCCTACCC | SEQ ID NO: 6147 |
| rs1555115 | GGATGGCTGTCACACCTGAAAGTG | SEQ ID NO: 1416 | GGATGGCTGTCACACCTGAAAGTC | SEQ ID NO: 3782 | TCCAGGGTAAGGTTTTCAG | SEQ ID NO: 6148 |
| rs2284174 | AAACCTGCACATATACCCCAGAATT | SEQ ID NO: 1417 | AAACCTGCACATATACCCCAGAATC | SEQ ID NO: 3783 | AAAATAAAAGTTGGGGAGGG | SEQ ID NO: 6149 |
| rs2239806 | CCTCCTATGATGCTTGTGTGAAACTT | SEQ ID NO: 1418 | CCTCCTATGATGCTTGTGTGAAACTC | SEQ ID NO: 3784 | GTGTTCTAACTGTTTCATAATATCTG | SEQ ID NO: 6150 |
| rs1610714 | GGATGTGTTGAGGCTGGACCAA | SEQ ID NO: 1419 | GGATGTGTTGAGGCTGGACCAG | SEQ ID NO: 3785 | TGTCCTCCTGAGGTCTGTC | SEQ ID NO: 6151 |
| rs2071269 | AGAGCGCGCTCATCTTTTTCCT | SEQ ID NO: 1420 | AGAGCGCGCTCATCTTTTTCCG | SEQ ID NO: 3786 | CTCCCGAACTAGGGACAAAG | SEQ ID NO: 6152 |
| rs1264302 | ATGCCTCATTTGCCTCTCGCT | SEQ ID NO: 1421 | ATGCCTCATTTGCCTCTCGCC | SEQ ID NO: 3787 | CTTTTCGACCACCATTTTG | SEQ ID NO: 6153 |
| rs1265099 | ATCGCTGGGGCAGGGCTAT | SEQ ID NO: 1422 | ATCGCTGGGGCAGGGCTAC | SEQ ID NO: 3788 | GTACTGTGTTCCCTTCTGCC | SEQ ID NO: 6154 |
| rs926070 | TGGTCCACAGAATTTTTATAGTTGGA | SEQ ID NO: 1423 | TGGTCCACAGAATTTTTATAGTTGGG | SEQ ID NO: 3789 | TATTCTATTGTATGATTTACTTGCC | SEQ ID NO: 6155 |
| rs1150738 | GGTGGAGGATCCTGGCTACCAA | SEQ ID NO: 1424 | GGTGGAGGATCCTGGCTACCAC | SEQ ID NO: 3790 | CTGAATCCGATACCGCTTCT | SEQ ID NO: 6156 |
| rs2075015 | AGGCCTGGCCCATCTGCAGA | SEQ ID NO: 1425 | AGGCCTGGCCCATCTGCAGG | SEQ ID NO: 3791 | GAGCCAGGCTGGTAGTGATAC | SEQ ID NO: 6157 |
| rs1576 | CAACCTTGACAGATGCTCCAGCTC | SEQ ID NO: 1426 | CAACCTTGACAGATGCTCCAGCTG | SEQ ID NO: 3792 | AATCCCCAGATGAGCAGCTA | SEQ ID NO: 6158 |
| rs2074482 | CGGGCCAACAATTACTCACATTTT | SEQ ID NO: 1427 | CGGGCCAACAATTACTCACATTTG | SEQ ID NO: 3793 | GTTCATTGTCCACAGTGTCACT | SEQ ID NO: 6159 |
| rs2076523 | GTCTGAGCATCGCATCCAAGATA | SEQ ID NO: 1428 | GTCTGAGCATCGCATCCAAGATG | SEQ ID NO: 3794 | AGATGGGCTGTTCTATGCG | SEQ ID NO: 6160 |
| rs2395182 | TCTGTTAGACATAGTCTCAGATGCCTATTT | SEQ ID NO: 1429 | TCTGTTAGACATAGTCTCAGATGCCTATTG | SEQ ID NO: 3795 | ATTACCGAGAGAGTAGTCTGACTG | SEQ ID NO: 6161 |
| rs241429 | GAGTGCACTCTGTACTAGACACAATAGAAA | SEQ ID NO: 1430 | GAGTGCACTCTGTACTAGACACAATAGAAG | SEQ ID NO: 3796 | CTACAATAGAAGGGAAAAGATATTG | SEQ ID NO: 6162 |
| rs565448 | TGGATTATGTTGTCCATCTAAACTTTTC | SEQ ID NO: 1431 | TGGATTATGTTGTCCATCTAAACTTTTTG | SEQ ID NO: 3797 | AACTTAGATTTTACTTAAAAAATTGC | SEQ ID NO: 6163 |
| rs1383265 | TCTGAATCACCACCTGGAGAAAAGT | SEQ ID NO: 1432 | TCTGAATCACCACCTGGAGAAAAGC | SEQ ID NO: 3798 | ACTCACAAATCTTCAATATCCTC | SEQ ID NO: 6164 |
| rs1150771 | TCCAAACCCTGTTCCAAAGCCT | SEQ ID NO: 1433 | TCCAAACCCTGTTCCAAAGCCC | SEQ ID NO: 3799 | GTCACCTACATGCCTGTCTGT | SEQ ID NO: 6165 |
| rs2284189 | CCTGCATCCCATACCGTTTCCA | SEQ ID NO: 1434 | CCTGCATCCCATACCGTTTCCG | SEQ ID NO: 3800 | CACTAGTCTGAGCACCCTCTG | SEQ ID NO: 6166 |
| rs3213484 | CCTCGAGGGCCTCCACCT | SEQ ID NO: 1435 | CCTCGAGGGCCTCCACCA | SEQ ID NO: 3801 | CAGGTAATATTTCAGCCATGAT | SEQ ID NO: 6167 |
| rs2856716 | CAATTTTTCTGATTGCCTCATATCAATA | SEQ ID NO: 1436 | CAATTTTTCTGATTGCCTCATATCAATT | SEQ ID NO: 3802 | GGTTCATATATTTCTTCAGCCTT | SEQ ID NO: 6168 |
| rs2076526 | CAGGGTTGCTGTGAGGCTCAA | SEQ ID NO: 1437 | CAGGGTTGOTGTGAGGCTCAG | SEQ ID NO: 3803 | GTCATCCTTAGGTGAGGTGG | SEQ ID NO: 6169 |
| rs3830122 | TGCTTATTCCAAATTGAACCTCTTTAA | SEQ ID NO: 1438 | TGCTTATTCCAAATTGAACCTCTTTAC | SEQ ID NO: 3804 | TGAGACTCTAAAGATTCAGAGCTTT | SEQ ID NO: 6170 |
| rs1882 | CACTGGCTCCACTGAGGGCA | SEQ ID NO: 1439 | CACTGGCTCCACTGAGGGCG | SEQ ID NO: 3805 | CTAGACTCTACAGCCAGGCG | SEQ ID NO: 6171 |
| rs512559 | ACTTTGCTGGGACAGGGAGGCT | SEQ ID NO: 1440 | ACTTTGCTGGGACAGGGAGGCC | SEQ ID NO: 3806 | TCAGTAAGAGGCTCAAGGG | SEQ ID NO: 6172 |
| rs2857102 | GTTCCATGGACTCTTAGGAATTAGACTA | SEQ ID NO: 1441 | GTTCCATGGACTCTTAGGAATTAGACTC | SEQ ID NO: 3807 | TACTACTACTAAGCATTCACTGGTG | SEQ ID NO: 6173 |
| rs2856698 | CTGCCTGGCCCAGCCTTCTG | SEQ ID NO: 1442 | CTGCCTGGCCCAGCCTTCTC | SEQ ID NO: 3808 | ACAGTTGACTACCCTGGCC | SEQ ID NO: 6174 |
| rs204989 | TTAAGTATTCACTATGCATCAAGTGCTTT | SEQ ID NO: 1443 | TTAAGTATTCACTATGCATCAAGTGCTTC | SEQ ID NO: 3809 | TTACATTATTAATACATCAACCCTATG | SEQ ID NO: 6175 |
| rs419788 | CACCAGTGTGCAGTGACCTGATTT | SEQ ID NO: 1444 | CACCAGTGTGCAGTGACCTGATTC | SEQ ID NO: 3810 | ATGTCCCTATCCTACAGATCTTC | SEQ ID NO: 6176 |
| rs986475 | CCTGAACACTGTCATTCACCCAAT | SEQ ID NO: 1445 | CCTGAACACTGTCATTCACCCAAC | SEQ ID NO: 3811 | AATTACTATTTGACCCCAGAGTG | SEQ ID NO: 6177 |
| rs2763979 | CAATTCCTGGTTCACTCCTGGACA | SEQ ID NO: 1446 | CAATTCCTGGTTCAGTCCTGGACG | SEQ ID NO: 3812 | TGTGTAGAAGTAGGACCTCACAG | SEQ ID NO: 6178 |
| rs1062470 | TCTTTGGTGAAGTAGCCCACAGGA | SEQ ID NO: 1447 | TCTTTGGTGAAGTAGCCCACAGGG | SEQ ID NO: 3813 | AGATTTTACCCTTACTGTAGGTCA | SEQ ID NO: 6179 |
| rs2523668 | CTGCTTTCCAGGCTTGGAGTCTT | SEQ ID NO: 1448 | CTGCTTTCCAGGCTTGGAGTCTG | SEQ ID NO: 3814 | CCTGTAGTACATCAATTCCAACA | SEQ ID NO: 6180 |
| rs2524069 | AGAACGTGAACAGGCCAAGAAAGA | SEQ ID NO: 1449 | AGAACGTGAACAGGCCAAGAAAGT | SEQ ID NO: 3815 | GATAAAGAAATCTCAGCAAAGAAG | SEQ ID NO: 6181 |
| rs3135339 | ACCTGTGGAAGTTGTGTCCACCG | SEQ ID NO: 1450 | ACCTGTGGAAGTTGTGTCCACCC | SEQ ID NO: 3816 | AGCTTAGAATCCTCATGTCATCT | SEQ ID NO: 6182 |
| rs206776 | CCTAATGGCAAGGTCCCATAAGCTT | SEQ ID NO: 1451 | CCTAATGGCAAGGTCCCATAAGCTC | SEQ ID NO: 3817 | TGCACACATTTTTAGAAAAGATT | SEQ ID NO: 6183 |
| rs1237485 | ATTAATGGGAAGGCTGCACGGT | SEQ ID NO: 1452 | ATTAATGGGAAGGCTGCACGGC | SEQ ID NO: 3818 | ACCGATTCTTTGAAAAATACTTG | SEQ ID NO: 6184 |
| rs805290 | TTGTGCAGGGTTGTATCCCCAA | SEQ ID NO: 1453 | TTGTGCAGGGTTGTATCCCCAG | SEQ ID NO: 3819 | ATCTGATGCAGTGATTCAATAAA | SEQ ID NO: 6185 |
| rs805268 | CCACCTCGTTCCACCCTTTAGTTAT | SEQ ID NO: 1454 | CCACCTCGTTCCACCCTTTAGTTAG | SEQ ID NO: 3820 | CTGACTTTAGGCTACTTTTCCTT | SEQ ID NO: 6186 |
| rs1345228 | GGTCACCTCCAGTGGAGTGGTA | SEQ ID NO: 1455 | GGTCACCTCCAGTGGAGTGGTG | SEQ ID NO: 3821 | GTCAAGAAATGTAATTTGTCCTTT | SEQ ID NO: 6187 |
| rs7742523 | GCCATTGGGAATAGTGCTGCTAT | SEQ ID NO: 1456 | GCCATTGGGAATAGTGCTGCTAC | SEQ ID NO: 3822 | AGCATGAGTGTACAATTATCTGTG | SEQ ID NO: 6188 |
| rs1053924 | GGGGGGACGTAAGGGAAGAATAATA | SEQ ID NO: 1457 | GGGGGGACGTAAGGGAAGAATAATG | SEQ ID NO: 3823 | TTATCTGGCTGTGTTTTTGTTA | SEQ ID NO: 6189 |
| rs1270942 | ACTGTCATCTCCCCATTGCCTTT | SEQ ID NO: 1458 | ACTGTCATCTCCCCATTGCCTTC | SEQ ID NO: 3824 | TCACTCAGTAGATGTCCCCTAA | SEQ ID NO: 6190 |
| rs3129872 | CCAAGCTCTTGGCCTGAGTTGT | SEQ ID NO: 1459 | CCAAGCTCTTGGCCTGAGTTGA | SEQ ID NO: 3825 | CATCTTGAAGTCTAGGACTCCA | SEQ ID NO: 6191 |
| rs805256 | ACTCAATGAGCAGGTCCCTCACTAT | SEQ ID NO: 1460 | ACTCAATGAGCAGGTCCCTCACTAC | SEQ ID NO: 3826 | GACAAGCTCTAGACATGATCTTG | SEQ ID NO: 6192 |
| rs3116998 | GGGGTCTGTCCTCGAAGCAGA | SEQ ID NO: 1461 | GGGGTCTGTCCTCGAAGCAGG | SEQ ID NO: 3827 | ACTGGGACTAAAGAAACAGAAAA | SEQ ID NO: 6193 |
| rs2517484 | TCTGTCCCTCCTTTCTCTGAATCCT | SEQ ID NO: 1462 | TCTGTCCCTCCTTTCTCTGAATCCC | SEQ ID NO: 3828 | TACTTAAATCACTCCCTGGCTAC | SEQ ID NO: 6194 |
| rs886398 | TACCTGCTGCTCATCCAGCCAGA | SEQ ID NO: 1463 | TACCTGCTGCTCATCCAGCCAGG | SEQ ID NO: 3829 | CAACAGGAAGTTGTCAATGATAT | SEQ ID NO: 6195 |
| rs984778 | ACCGTCATCCCTACCCAAGGAT | SEQ ID NO: 1464 | ACCGTCATCCCTACCCAAGGAC | SEQ ID NO: 3830 | TGTTTGTGGTATGAGTGGTTTTA | SEQ ID NO: 6196 |
| rs1819784 | GGGACAGCAAACCAAGACAAAAAT | SEQ ID NO: 1465 | GGGACAGCAAACCAAGACAAAAAC | SEQ ID NO: 3831 | GTTAGTTGTCTCAAAGACAAAAAC | SEQ ID NO: 6197 |
| rs537757 | AAGGGAATGCCCAGTACCTACTGAA | SEQ ID NO: 1466 | AAAGGGAATGCCCAGTACCTACTGAT | SEQ ID NO: 3832 | TGTTTGCCTTTATCTCTTTCAG | SEQ ID NO: 6198 |
| rs213199 | CTGTTGCTCGTTCATTGCCCAA | SEQ ID NO: 1467 | CTGTTGCTCGTTCATTGCCCAG | SEQ ID NO: 3833 | AGAAACTGATAGAAGAACCTAGGG | SEQ ID NO: 6199 |
| rs7762289 | ACATCTTAGCGTCCAGATGACCCA | SEQ ID NO: 1468 | ACATCTTAGCGTCCAGATGACCCC | SEQ ID NO: 3834 | CATATAGGTGTCCTTTAGTATGGA | SEQ ID NO: 6200 |
| rs1264709 | TGGAGCTAGGATTCTGGACATGAAT | SEQ ID NO: 1469 | TGGAGCTAGGATTCTGGACATGAAA | SEQ ID NO: 3835 | AAGTCCTTCCAACTAAATGTATTT | SEQ ID NO: 6201 |
| rs2071352 | GGAAAATGGGACCAAGAGGTCCT | SEQ ID NO: 1470 | GGAAAATGGGACCAAGAGGTCCA | SEQ ID NO: 3836 | GATAACCTTGGACAGACAAGGT | SEQ ID NO: 6202 |
| rs2213584 | TAAGGCCAGTCTTCAGTGTCCAAT | SEQ ID NO: 1471 | TAAGGCCAGTCTTCAGTGTCCAAC | SEQ ID NO: 3837 | TCTTTGCTCATTAAATCTGGAAT | SEQ ID NO: 6203 |
| rs7382138 | CTATGCATGCATGTGGCCGTT | SEQ ID NO: 1472 | CTATGCATGCATGTGGCCGTC | SEQ ID NO: 3838 | GAACTTTCTGGAGTATATTAAGGC | SEQ ID NO: 6204 |
| rs660550 | CACTCTTGGAGTCCTGGGGGT | SEQ ID NO: 1473 | CACTCTTGGAGTCCTGGGGGG | SEQ ID NO: 3839 | GATGGAAAACAAAGCAATACTT | SEQ ID NO: 6205 |
| rs3094626 | CTGAGTTTGCAGTGGAGAGTGAAAT | SEQ ID NO: 1474 | CTGAGTTTGCAGTGGAGAGTGAAAC | SEQ ID NO: 3840 | GCATGATAGTTACAAAATTGTCAG | SEQ ID NO: 6206 |
| rs130078 | CTGGCTGTGGACCTCAGAAGGG | SEQ ID NO: 1475 | CTGGCTGTGGACCTCAGAAGGC | SEQ ID NO: 3841 | CTTGTTCCCCAACATATTTTCT | SEQ ID NO: 6207 |
| rs1610647 | CCCTTACGGGTGTGTCCCCTT | SEQ ID NO: 1476 | CCCTAACGGGTGTGTCCCCTC | SEQ ID NO: 3842 | TAAGATGTCGCAGACTTTAAGC | SEQ ID NO: 6208 |
| rs2071594 | TCACTGCTACAACATGCAAGGAACTC | SEQ ID NO: 1477 | TCACTGCTACAACATGCAAGGAACTG | SEQ ID NO: 3843 | GCTAGACTTTACAGAATGATTCCT | SEQ ID NO: 6209 |
| rs393603 | CCCTCACCCCATTGCAAGTGT | SEQ ID NO: 1478 | CCCTCACCCCATTGCAAGTGA | SEQ ID NO: 3844 | AGTAGAATTATCCAGCAATTCAG | SEQ ID NO: 6210 |
| rs1034325 | TGCACTCCTAAGCTGCATATGCTTT | SEQ ID NO: 1479 | TGCACTCCTAAGCTGCATATGCTTC | SEQ ID NO: 3845 | GGTGATACTCTAAGAAGCCCAC | SEQ ID NO: 6211 |
| rs971570 | CAGGAATGGAGAGAAGGGTAATGGTT | SEQ ID NO: 1480 | CAGGAACTGGAGAGAAGGGTAATGGTG | SEQ ID NO: 3846 | TACCTCTTATTGTGGAAACCTGT | SEQ ID NO: 6212 |
| rs3115635 | CCATGTATTTGACTTGCAGCAGGT | SEQ ID NO: 1481 | CCATGTATTTGACTTGCAGCAGGC | SEQ ID NO: 3847 | CACAACAAATGTCAGGTGAATTA | SEQ ID NO: 6213 |
| rs2524172 | CACCCCATGCTTTACAGAACTGAA | SEQ ID NO: 1482 | CACCCCATGCTTTACAGAACTGAG | SEQ ID NO: 3848 | TCCACAGTCATACTCATCTCTC | SEQ ID NO: 6214 |
| rs2523746 | TACCCCAGGCAGCAATTGTGCT | SEQ ID NO: 1483 | TACCCCAGGCAGCAATTGTGCC | SEQ ID NO: 3849 | AGTACTCTGATGCATCTCATGAT | SEQ ID NO: 6215 |
| rs1362069 | GTGGCTCCTTACAATTCTCTGTGGT | SEQ ID NO: 1484 | GTGGCTCCTTACAATTCTCTGTGGC | SEQ ID NO: 3850 | TTCTTCCAATGTAAGTAGTTCTTG | SEQ ID NO: 6216 |
| rs3129203 | CTCATCTCCACCCCCACCTGT | SEQ ID NO: 1485 | CTCATCTCCACCCCCACCTGC | SEQ ID NO: 3851 | CTGGTCCATTCAAAACTACAAA | SEQ ID NO: 6217 |
| rs3871248 | GGGCCAAGAGGAAATACTCAGTTTTA | SEQ ID NO: 1486 | GGGCCAAGAGGAAATACTCAGTTTTG | SEQ ID NO: 3852 | ATGTGTCAGATTTAAGGCCATAT | SEQ ID NO: 6218 |
| rs4248157 | TGTGTGTCTGGGAGTGAGAACTTCT | SEQ ID NO: 1487 | TGTGTGTCTGGGAGTGAGAACTTCC | SEQ ID NO: 3853 | AGTCTATCTAAGGAATGGAGGG | SEQ ID NO: 6219 |
| rs3130140 | GTCTGTCCTCAGCAGTCTTTCCCT | SEQ ID NO: 1488 | GTCTGTCCTCAGCAGTCTTTCCCC | SEQ ID NO: 3854 | CTGACCTATGACTTCTGGGAAT | SEQ ID NO: 6220 |
| rs2524100 | TGGGGAACACCATTGAAGTAAAATCA | SEQ ID NO: 1489 | TGGGGAACACCATTGAAGTAAAATCG | SEQ ID NO: 3855 | AAAAATATCTGAGCATATGCTTC | SEQ ID NO: 6221 |
| rs1800625 | GAACAGGAGAGAAACCTGTTTGGAA | SEQ ID NO: 1490 | GAACAGGAGAGAAACCTGTTTGGAG | SEQ ID NO: 3856 | TTCGTGAAAGAAAATCATTTTT | SEQ ID NO: 6222 |
| rs2239709 | CCAAGTCCTTCATTTTGTCCAGGA | SEQ ID NO: 1491 | CCAAGTCCTTCATTTTGTCCAGGG | SEQ ID NO: 3857 | TGTAGTAGTTACGTGACCCGAA | SEQ ID NO: 6223 |
| rs241439 | TGAATTCTTCAGCCTCCCTCTTGA | SEQ ID NO: 1492 | TGAATTCACAGCCTCCCTCTTGC | SEQ ID NO: 3858 | CAAGAGTCTTTGTTTGCAGAGA | SEQ ID NO: 6224 |
| rs6907291 | GGGAAATAATGCCCACAGGACTCA | SEQ ID NO: 1493 | GGGAAATAATGCCCACAGGACTCC | SEQ ID NO: 3859 | CTAGTCCTTTGAGTTGTGCTG | SEQ ID NO: 6225 |
| rs6917477 | CAGGCCAATCTGGAAGATAAAAAAGC | SEQ ID NO: 1494 | CAGGCCAATCTGGAAGATAAAAAAGG | SEQ ID NO: 3860 | AAGGTAGATTCAAATGTAGGAGG | SEQ ID NO: 6226 |
| rs6923504 | CTCCATCCAAAAGACACTTCTGCTC | SEQ ID NO: 1495 | CTCCATCCAAAAGACACTTCTGCTG | SEQ ID NO: 3861 | CTCCTATCATGCCACACTCTAC | SEQ ID NO: 6227 |
| rs6906021 | CTCCCAGACTCTGGCTTCATGAT | SEQ ID NO: 1496 | CTCCCAGACTCTGGCTTCATGAC | SEQ ID NO: 3862 | AAACTCAGAGACTGAGATGTAGAC | SEQ ID NO: 6228 |
| rs2849015 | TGCCAGGTAGGATGCTATCACAATAT | SEQ ID NO: 1497 | TGCCAGGTAGGATGCTATCACAATAC | SEQ ID NO: 3863 | CCTAATTAGAGATGGTAGGGGT | SEQ ID NO: 6229 |
| rs2517462 | GCTACTCCTCGAGCTGTCTGACCA | SEQ ID NO: 1498 | GCTACTCCTCGAGCTGTCTGACCG | SEQ ID NO: 3864 | GCCTCTATCTGTTTTCCTCTG | SEQ ID NO: 6230 |
| rs7382297 | AAATAGCTTTGCCCTGTCATTTCCA | SEQ ID NO: 1499 | AAATAGCTTTGCCCTGTCATTTCCC | SEQ ID NO: 3865 | AAGAAAGCACTGCAGTTACTCA | SEQ ID NO: 6231 |
| rs6457713 | CCACTATAACCTACGGGGGAAAAAAA | SEQ ID NO: 1500 | CCACTATAACCTACGGGGGAAAAAAG | SEQ ID NO: 3866 | AACATAACCATGAACCAACTAAT | SEQ ID NO: 6232 |
| rs1075496 | GCTTTTTGCAACTGCCTAACATGTGT | SEQ ID NO: 1501 | GCTTTTTGCAACTGCCTAACATGTGG | SEQ ID NO: 3867 | GTTCTATAAATCCTCTTCCCCA | SEQ ID NO: 6233 |
| rs3131083 | GGTCTCAGCGATGATCAAGATATCAT | SEQ ID NO: 1502 | GGTCTCAGCGATGATCAAGATATCAC | SEQ ID NO: 3868 | TTTTGACACCACATTCAATGTAT | SEQ ID NO: 6234 |
| rs2256965 | CATCACTTCCTAAGCTCCCAGGACT | SEQ ID NO: 1503 | CATCACTTCCTAAGCTCCCAGGACC | SEQ ID NO: 3869 | TCTCTATGCAGATGCAAGAGAC | SEQ ID NO: 6235 |
| rs3093662 | TGAATGCCTGGAAGGTGAATACACA | SEQ ID NO: 1504 | TGAATGCCTGGAAGGTGAATACACG | SEQ ID NO: 3870 | ATGAATGGAGAGAGAAAACCAG | SEQ ID NO: 6236 |
| rs2395305 | CAATCAATTAAATGGTTTGCTGCCT | SEQ ID NO: 1505 | CAATCAATTAAATGGTTTGCTGCCA | SEQ ID NO: 3871 | TTTTCTCTTTCTATCAATTCAAGTC | SEQ ID NO: 6237 |
| rs3763304 | TGCTCACTGGAATCTCCATGAGCT | SEQ ID NO: 1506 | TGCTCACTGGAATCTCCATGAGCC | SEQ ID NO: 3872 | CCAAAAGTGTCAGGTAACACAG | SEQ ID NO: 6238 |
| rs2227126 | CCACGAATCCAGTGTAGGACACAA | SEQ ID NO: 1507 | CCACGAATCCAGTGTAGGACAGAG | SEQ ID NO: 3873 | ACGCAGTACTGCTGCTACATT | SEQ ID NO: 6239 |
| rs261946 | CCTCTGGAAGCTACAGGTCTGTTTCA | SEQ ID NO: 1508 | CCTCTGGAAGCTACAGGTCTGTTTCG | SEQ ID NO: 3874 | ATGGATCTATTCTTTTTCCTGG | SEQ ID NO: 6240 |
| rs4987411 | CTCCGATCCTAGAGTCCATTCCCT | SEQ ID NO: 1509 | CTCCGATGCTAGACTCCATTCCCC | SEQ ID NO: 3875 | ATGTATTTCTTCGTGGGAAAC | SEQ ID NO: 6241 |
| rs1633017 | AGGACCACGAGGCCATGAAAAT | SEQ ID NO: 1510 | AGGACCACGAGGCCATGAAAAC | SEQ ID NO: 3876 | GCTTTGGCTACAAATAGGTCAT | SEQ ID NO: 6242 |
| rs7382061 | CTGGACTTGGCCTCACGGCTA | SEQ ID NO: 1511 | CTGGACTTGGCCTCACGGCTG | SEQ ID NO: 3877 | TATAGGGTTAAATGAGAGAATGGA | SEQ ID NO: 6243 |
| rs3129980 | CTGAGGCCATCAGCACTGCAG | SEQ ID NO: 1512 | CTGAGGCCATCAGCACTGCAC | SEQ ID NO: 3878 | CACCTGGAACAGTCTTGTTAAA | SEQ ID NO: 6244 |
| rs3734838 | GGATTTGTGGTCCTTGGATTCACA | SEQ ID NO: 1513 | GGATTTGTGGTCCTTGGATTCACG | SEQ ID NO: 3879 | CACACAAAACAGAGGAACTTC | SEQ ID NO: 6245 |
| rs3129073 | GGGGTATGCTATGGAATAGGGTGAT | SEQ ID NO: 1514 | GGGGTATGCTATGGAATAGGGTGAC | SEQ ID NO: 3880 | GAACTTTGTGGATGACATACCA | SEQ ID NO: 6246 |
| rs1063355 | CTGCTCAAGAGAAGCACATGAAAAA | SEQ ID NO: 1515 | CTGCTCAAGAGAAGCACATGAAAAC | SEQ ID NO: 3881 | ATTACCTGACTTTAGAGCTTTTTT | SEQ ID NO: 6247 |
| rs994321 | GCCCCAGATTGGGTGTCTAAGAAT | SEQ ID NO: 1516 | GCCCCAGATTCCGTGTCTAAGAAC | SEQ ID NO: 3882 | CAATGTCCCCTAGTTAATATATTCT | SEQ ID NO: 6248 |
| rs3129809 | TTTTCTGCCTCTCACTGGAATTTTA | SEQ ID NO: 1517 | TTTTCTGCCTCTCACTGGAATTTTG | SEQ ID NO: 3583 | CCCTACACTTTGAACTCATTTC | SEQ ID NO: 6249 |
| rs7766854 | GGCATGTCTCACATGATGGCAGA | SEQ ID NO: 1518 | GGCATGTCTCACATGATGGCAGG | SEQ ID NO: 3884 | AAGAGAAGAATGACAGCAAGC | SEQ ID NO: 6250 |
| rs3763288 | GCTGTGCAGTAATCTAGGCTGAAGGA | SEQ ID NO: 1519 | GCTGTGCAGTAATCTAGGCTGAAGGG | SEQ ID NO: 3885 | GGGGAAAGCCTAGACTAAGATT | SEQ ID NO: 6251 |
| rs630379 | TCCTACTAAGTGAAATGTGGCATGGT | SEQ ID NO: 1520 | TCCTACTAAGTGAAATGTGGCATGGG | SEQ ID NO: 3886 | TATGTCTTGTGACTATGATTTGTG | SEQ ID NO: 6252 |
| rs2844845 | GCAGACTACATGCAAAAGCTATGGTGT | SEQ ID NO: 1521 | GCAGACTACATGCAAAAGCTATGGTGA | SEQ ID NO: 3887 | ATCTGAAATTCTGGAGGTATCAG | SEQ ID NO: 6253 |
| rs3130666 | TTTTTTGGTTCCTCCGACTTGTCTA | SEQ ID NO: 1522 | TTTTTTGGTTCCTCCGACTTGTCTG | SEQ ID NO: 3888 | ATCTCTTGTAGCTCAGGTTCC | SEQ ID NO: 6254 |
| rs2021723 | GCTGGCCATCTCTCCAGGTCTA | SEQ ID NO: 1523 | GCTGGCCATCTCTCCAGGTCTG | SEQ ID NO: 3889 | AGCCAGAGAAGATGATCTGAA | SEQ ID NO: 6255 |
| rs1634718 | TGAAGGGCTACGTGGAGTGTAAGGA | SEQ ID NO: 1524 | TGAAGGGCTACGTGGAGTGTAAGGG | SEQ ID NO: 3890 | CTGGTAATTTTGGCATGTGTTA | SEQ ID NO: 6256 |
| rs1264539 | CATGCATCTGTTGACATAACTGCAT | SEQ ID NO: 1525 | CATGCATCTGTTGACATAACTGCAC | SEQ ID NO: 3891 | TTTTCTACCATTTCTATTCATG | SEQ ID NO: 6257 |
| rs2844748 | TAGAACCCTGCTGGGAGATTGCT | SEQ ID NO: 1526 | TAGAACCCTGCTGGGAGATTGCA | SEQ ID NO: 3892 | TCATTCTCCAGAACCTCTGAG | SEQ ID NO: 6258 |
| rs805292 | CCCCCACTCGGTCTTTCCAATA | SEQ ID NO: 1527 | CCCCCACTCGGTCTTTCCAATG | SEQ ID NO: 3893 | CTGCTCAGCAATAAGTGACTC | SEQ ID NO: 6259 |
| rs7741884 | CTGGTACCTGCGCATTGCAGA | SEQ ID NO: 1528 | CTGGTACCTGCGCATTGCAGC | SEQ ID NO: 3894 | TCTCCTTCTCATTCTTAAGGC | SEQ ID NO: 6260 |
| rs2245420 | TGAACACCACTGGAGAGTTTCAAAC | SEQ ID NO: 1529 | TGAACACCACTGGAGAGTTTCAAAG | SEQ ID NO: 3895 | ACTGTCACCAAACAGGTACTGAT | SEQ ID NO: 6261 |
| rs259940 | GCAGACACTGATGGTCTTCAGATCACA | SEQ ID NO: 1530 | GCAGACACTGATGGTCTTCAGATCACG | SEQ ID NO: 3896 | TTTTAGTAGCAAGAATGTAGACCA | SEQ ID NO: 6262 |
| rs2853935 | TCAAAGCCTGGGACATTTATTTGT | SEQ ID NO: 1531 | TCAAAGCCTGGGACATTTATTTGC | SEQ ID NO: 3897 | CTAAACAAGCAGTTATTCTTCATC | SEQ ID NO: 6263 |
| rs6911467 | GGTGGCACTTGTCTGCAGTCCTA | SEQ ID NO: 1532 | GGTGGCACTTGTCTGCAGTCCTG | SEQ ID NO: 3898 | CTACTCTGAAGACTGAGGCAG | SEQ ID NO: 6264 |
| rs3132712 | CCAAACCTGCTTCCATCTTTTGATT | SEQ ID NO: 1533 | CCAAACCTGCTTCCATCTTTTGATC | SEQ ID NO: 3899 | CTGGACACACATGTTCTTCCTA | SEQ ID NO: 6265 |
| rs3763317 | AAGGGTGTTGCTGATGCCTCCT | SEQ ID NO: 1534 | AAGGGTGTTGCTGATGCCTCCC | SEQ ID NO: 3900 | GACTCCTTTTGTGAAAAGCAG | SEQ ID NO: 6266 |
| rs2853939 | CCCTAGCCAGTCTTGAGTCTCATCATT | SEQ ID NO: 1535 | CCCTAGCCAGTCTTGAGTCTCATCATC | SEQ ID NO: 3901 | TACGATTTCAGAACTATTGTGAAT | SEQ ID NO: 6267 |
| rs605203 | GGGAGTGTACGGCAGTTAAGGTAGTTT | SEQ ID NO: 1536 | GGGAGTGTACGGCAGTTAAGGTAGTTG | SEQ ID NO: 3902 | CGAGATTATAAAGCGTTTTCTTA | SEQ ID NO: 6268 |
| rs6457594 | GGGGCCATTGAACCACACATACA | SEQ ID NO: 1537 | GGGGCCATTGATCCACACATACG | SEQ ID NO: 3903 | GTTTGTCACTGGCCATTCTAAT | SEQ ID NO: 6269 |
| rs2144014 | TTGTTCTTCCTCAAGAATGCCAATT | SEQ ID NO: 1538 | TTGTTCTTCCTCAAGAATGCCAATC | SEQ ID NO: 3904 | TTCTTAGGTCATTTAACATAATCCC | SEQ ID NO: 6270 |
| rs2534790 | CCACAAGACAGTTACCTAGGATGCCTT | SEQ ID NO: 1539 | CCACAAGACAGTTACCTAGGATGCCTG | SEQ ID NO: 3905 | CTCCTTTACACTTCTACTCCCC | SEQ ID NO: 6271 |
| rs986522 | TGTTTTCAGCAGGGGTAGTGGTC | SEQ ID NO: 1540 | TGTTTTCACCAGGGGTAGTGGTG | SEQ ID NO: 3906 | TTACTGACAAAGCAGAATGGAA | SEQ ID NO: 6272 |
| rs805264 | CCTCCACCATAGGCTTCCCCT | SEQ ID NO: 1541 | CCTCCACCATAGGCTTCCCCC | SEQ ID NO: 3907 | ATCTCAGTCCATACCTGCTTC | SEQ ID NO: 6273 |
| rs7757269 | CAACTCTTAATTGGCCCTCAAAACTTA | SEQ ID NO: 1542 | CAACTCTTAATTGGCCCTCAAAACTTG | SEQ ID NO: 3908 | CCTACTTGCTTCTATCTTCAGAA | SEQ ID NO: 6274 |
| rs916570 | GCAAGCTCCCAAAGCATATTCCT | SEQ ID NO: 1543 | GCAAGCTCCCAAAGCATATTCCC | SEQ ID NO: 3909 | TAGAAACTCTGGGGTCATGAA | SEQ ID NO: 6275 |
| rs3093668 | CAAGGGACTTCCAGAGATTCGGG | SEQ ID NO: 1544 | CAAGGGACTTCCAGAGATTCGGC | SEQ ID NO: 3910 | GTCTAGGCTTCAAATCACCCT | SEQ ID NO: 6276 |
| rs928824 | TTGTTGGGTGATATGGTAAATGGATA | SEQ ID NO: 1545 | TTGTTGGGTGATATGGTAAATGGATG | SEQ ID NO: 3911 | GTAGCTGTTTAAGATATTGCCAT | SEQ ID NO: 6277 |
| rs3132682 | CAACTGAAAAAGGCAGGGTTGAC | SEQ ID NO: 1546 | CAACTGAAAAAGGCAGGGTTGAG | SEQ ID NO: 3912 | CACAGCAAGAACACAAGTCAG | SEQ ID NO: 6278 |
| rs4248148 | CCTAAAACACTCCCTCGGGCTAT | SEQ ID NO: 1547 | CCTAAAACACTCCCTCGGGCTAC | SEQ ID NO: 3913 | CCTTTGTAGTCCCATCCTCTT | SEQ ID NO: 6279 |
| rs707928 | CTGTGTTTTCCAGCACAGCCCT | SEQ ID NO: 1548 | CTGTGTTTTCCAGCACAGCCCC | SEQ ID NO: 3914 | GTTTCAAACATTCAGGCTCAC | SEQ ID NO: 6280 |
| rs3130375 | TTTATGGGCAGAACATTTGAAAGAAT | SEQ ID NO: 1549 | TTTATGGGCAGAACATTTGAAAGAAG | SEQ ID NO: 3915 | TAAATACTCAACAGGGAGCAGA | SEQ ID NO: 6281 |
| rs3097644 | GCATAGTTTTGAGTGTGCCTCTTTC | SEQ ID NO: 1550 | GCATAGTTTTGAGTGTGCCTCTTTG | SEQ ID NO: 3916 | AGGATGTATTTGAGTTCCGTTAT | SEQ ID NO: 6282 |
| rs1233397 | TGGCTACAAGTTTATGCCCCAGTAA | SEQ ID NO: 1551 | TGGCTACAAGTTTATGCCCCAGTAG | SEQ ID NO: 3917 | AATCAGTTCCAGTGCTCTTCAT | SEQ ID NO: 6283 |
| rs2023371 | TGATCCAGCAGGAAAAAAGAGGAT | SEQ ID NO: 1552 | TGATCCAGCAGGAAAAAAGAGGAC | SEQ ID NO: 3918 | CTCTCTAGTGGGGACACTCCT | SEQ ID NO: 6284 |
| rs2071540 | CGCACTTTTCAGCTGCGGA | SEQ ID NO: 1553 | CGCACTTTTCAGCTGCGGG | SEQ ID NO: 3919 | GTCTCCTCTTTTATCATCCTCAA | SEQ ID NO: 6285 |
| rs4713603 | TTCAGTGTTTGTCTCTTGCAGAAACA | SEQ ID NO: 1554 | TTCAGTGTGTCTCTTGCAGAAACG | SEQ ID NO: 3920 | GTTTGCTCACTTTGACTACCATA | SEQ ID NO: 6286 |
| rs3094014 | CATGTGGGATCCACAACACTTTAATTT | SEQ ID NO: 1555 | CATGTGGGATCCACAACACTTTAATTC | SEQ ID NO: 3921 | TAGTTTGCCTCAGGGCTATTATA | SEQ ID NO: 6287 |
| rs2856726 | TCTAGATTCGTGCCTGAATGAAATTTA | SEQ ID NO: 1556 | TCTAGATTCGTGCCTGAATGAAATTTT | SEQ ID NO: 3922 | TGGGTTTATAAGTATGGGTCTAGT | SEQ ID NO: 6288 |
| rs2735005 | ACCTGGGCAATGAAGTCAACACTA | SEQ ID NO: 1557 | ACCTGGGCAATGAAGTCAACACTG | SEQ ID NO: 3923 | GAATATCATCAATTTATGGAATACTG | SEQ ID NO: 6289 |
| rs241414 | ATTCCACTGATCTTGATGTTGCAT | SEQ ID NO: 1558 | ATTCCACTGATCTTGATGTTGCAG | SEQ ID NO: 3924 | GAATTCCACATTATGATTATGCC | SEQ ID NO: 6290 |
| rs2516400 | GTGGGCAGTGGGTTTTGCCT | SEQ ID NO: 1559 | GTGGGCAGTGGGTTTTGCCC | SEQ ID NO: 3925 | AGTAAAAACTCAGATAAACAACACC | SEQ ID NO: 6291 |
| rs2517416 | CGCTCATCTCCATGGCTATCGAT | SEQ ID NO: 1560 | CGCTCATCTCCATGGCTATCGAG | SEQ ID NO: 3926 | ATACTGGTCTCCTCCAGAACC | SEQ ID NO: 6292 |
| rs3749971 | CCAAAAAGTGGAAGAAGTGTAGCTGGA | SEQ ID NO: 1561 | CCAAAAAGTGGAAGAAGTGTAGCTGGG | SEQ ID NO: 3927 | GATACAGCCTAGAAAAGATATAGCC | SEQ ID NO: 6293 |
| rs6928431 | GCTGGGAACAGGGAGAATAAGAGAAGT | SEQ ID NO: 1562 | GCTGGGAACAGGGAGAATAAGAGAAGA | SEQ ID NO: 3928 | GATTAGAACAATTCTTTATTCACCA | SEQ ID NO: 6294 |
| rs6911693 | GATTAGCATAGCCATTGTGGGAAACA | SEQ ID NO: 1563 | GATTAGCATAGCCATTGTGGGAAACG | SEQ ID NO: 3929 | TATGGAGGTTCTGCAAAAAA | SEQ ID NO: 6295 |
| rs1269852 | TCTGAGGCTCCCTTGCCCCG | SEQ ID NO: 1564 | TCTGAGGCTCCCTTGCCCCC | SEQ ID NO: 3930 | TTTCAGCTGTAGCCTCTTAGTT | SEQ ID NO: 6296 |
| rs241457 | AAATGAGTTGATGGAACACCTCCA | SEQ ID NO: 1565 | AAATGAGTTGATTTGGAACACCTCCC | SEQ ID NO: 3931 | AATTTCTTCAGAAGCATCCTTG | SEQ ID NO: 6297 |
| rs2077102 | GTGAGCAAGGGTCGGGGAT | SEQ ID NO: 1566 | GTGAGCAAGGGTCGGGGAG | SEQ ID NO: 3932 | TCTAGTGCGTAACAGTCTAGGG | SEQ ID NO: 6298 |
| rs1736995 | AGCACAGGGGCTTTGGGTTGT | SEQ ID NO: 1567 | AGCACAGGGGCTTTGGGTTGC | SEQ ID NO: 3933 | TTGATCAAAGTTGTATCTACAGAA | SEQ ID NO: 6299 |
| rs241413 | TTGATGCTTCCTAACCATGAATGTA | SEQ ID NO: 1568 | TTGATGCTTCCTAACCATGAATGTG | SEQ ID NO: 3934 | CTCTCCATCTGTGCAGTACTTC | SEQ ID NO: 6300 |
| rs1362074 | ATGAAGGACTGCCTTCAAATAATTCAT | SEQ ID NO: 1569 | ATGAAGGACTGCCTTCAAATAATTCAC | SEQ ID NO: 3935 | ATAACTTTCTTGAATGACCATGT | SEQ ID NO: 6301 |
| rs2524222 | TGAGATAGAAATGGTTCTTTGTGGCA | SEQ ID NO: 1570 | TGAGATAGAAATGGTTCTTTGTGGCG | SEQ ID NO: 3936 | TCACAATTTATTGGAGAATGGA | SEQ ID NO: 6302 |
| rs2857768 | AGGCGTTCGAATATTGTTTCCTCT | SEQ ID NO: 1571 | AGGCGTTCGAAAATTGTTTCCTCC | SEQ ID NO: 3937 | TCTCCATTCAACACTGAGGAC | SEQ ID NO: 6303 |
| rs2394390 | AGCAGGCCTCTCGAGGGCA | SEQ ID NO: 1572 | AGCAGGCCTCTCGAGGGCG | SEQ ID NO: 3938 | CCATGTTTAGATTCTGAGATGG | SEQ ID NO: 6304 |
| rs3218819 | CTGCAGCTCCGACTCTGAGCCA | SEQ ID NO: 1573 | CTGCAGCTCCGACTCTGAGCCG | SEQ ID NO: 3939 | GGATAGAAAGAGGATGAGGAG | SEQ ID NO: 6305 |
| rs2736176 | TTGTGAAATGCAGTACTGTTTACCCTCG | SEQ ID NO: 1574 | TTGTGAAATGCAGTACTGTTACCCTCC | SEQ ID NO: 3940 | TTGTCTCTTACTACCCGATTCTA | SEQ ID NO: 6306 |
| rs885933 | GCCAGTCATAGCTGTCAACAATCCA | SEQ ID NO: 1575 | GCCAGTCATAGCTGTCAACTATCCG | SEQ ID NO: 3941 | AGATTCTCATTATCTTTTTACACAGC | SEQ ID NO: 6307 |
| rs2517889 | AGGTACTCAACCCGCTCTGGGA | SEQ ID NO: 1576 | AGGTACTCAACCCGCTCTGGGG | SEQ ID NO: 3942 | TACAGCAGGAAAGCAGAGTGT | SEQ ID NO: 6308 |
| rs2207337 | CCAAAGATGAAATGTCCCAGGAGT | SEQ ID NO: 1577 | CCAAAGATGAAATGTCCCAGGAGA | SEQ ID NO: 3943 | TATTCTGAAAGAAATAATTTTGACTC | SEQ ID NO: 6309 |
| rs4713293 | CATGATCCTGGTGATTTCAAAGACTAT | SEQ ID NO: 1578 | CATGATCCTGGTGATTTCAAAGACTAC | SEQ ID NO: 3944 | TACATGATCATTTAACACCAAGG | SEQ ID NO: 6310 |
| rs2040410 | CCTCATTAGGGAGTTGTGAGGAATA | SEQ ID NO: 1579 | CCTCATTAGGCAGTTGTGAGGAATG | SEQ ID NO: 3945 | ACATATACTGTACTTGGAATGAAAA | SEQ ID NO: 6311 |
| rs1557611 | GAGGGGAGGAGAAAGAGCCTAGTGT | SEQ ID NO: 1580 | GAGGGGAGGAGAAAGAGCCTAGTGC | SEQ ID NO: 3946 | GATGTTCAGAAAAAAGGTATAGTT | SEQ ID NO: 6312 |
| rs3129942 | TGCTGAAAGTTTTTTGATCACTCACTA | SEQ ID NO: 1581 | TGCTGAAAGTTTTTTGATCACTCACTC | SEQ ID NO: 3947 | TAAAAATGTTTTGAGTATGCATTG | SEQ ID NO: 6313 |
| rs3094138 | AGAAAAGAACTTTGGTTGTGGTCAT | SEQ ID NO: 1582 | AGTAAAGAACTTTGGTTGTGGTCAC | SEQ ID NO: 3948 | GATGCATGAAACAAATAAACAAG | SEQ ID NO: 6314 |
| rs3129304 | CCGTAATCTGTATTGCTCATTTTGGTCT | SEQ ID NO: 1583 | CCGTAATCTGTATTGCTCATTTTGGTCC | SEQ ID NO: 3949 | GACATAAACTATGTGGTATGGTGA | SEQ ID NO: 6315 |
| rs2071288 | TGATCCTCCCACAGAGCCTGTAT | SEQ ID NO: 1584 | TGATCCTCCCACAGAGCCTGTAC | SEQ ID NO: 3950 | GAGACAGGGAAAATTGAGAGC | SEQ ID NO: 6316 |
| rs6921644 | CCAAGGTGCTATCTGGTCCTATAGCT | SEQ ID NO: 1585 | CCAAGGTGCTATCTGGTCCTATAGCG | SEQ ID NO: 3951 | GCTTCTACAGTTTCCTTCTAATATC | SEQ ID NO: 6317 |
| rs206777 | CGTGTGCTCTGCTGTTCCTTTAAT | SEQ ID NO: 1586 | CGTGTGCTCTGCTGTTCCTTTAAC | SEQ ID NO: 3952 | TGTGGAGACAAGAACCTTTTG | SEQ ID NO: 6318 |
| rs17719 | GGGCTGTCACGGAGCGACTA | SEQ ID NO: 1587 | GGGCTGTCACGGAGCGACTG | SEQ ID NO: 3953 | CGAGATCGCCTAGTATGTTCT | SEQ ID NO: 6319 |
| rs1024470 | TCTTGACCTAACATTTCCATGTCTGA | SEQ ID NO: 1588 | TCTTGACCTAACATTTCCATGTCTGG | SEQ ID NO: 3954 | AGTTTGTCCTACAAATAGACTTGC | SEQ ID NO: 6320 |
| rs620202 | TACAGCCCTATGGGGATGGGGA | SEQ ID NO: 1589 | TACAGCCCTATGGGGATGGGGC | SEQ ID NO: 3955 | TAAGCAAAATGGGTGGAGTTA | SEQ ID NO: 6321 |
| rs409558 | GCGCGCAGAATGCAAAGCA | SEQ ID NO: 1590 | GCGCGCAGAATGCAAAGCG | SEQ ID NO: 3956 | TAAGTACTTCTGCTACAGGGCT | SEQ ID NO: 6322 |
| rs3117330 | TGAAATTGAAAAGTTGAGGCATTTTTTT | SEQ ID NO: 1591 | TGAAATTGAAAAGTTGAGGCATTTTTTC | SEQ ID NO: 3957 | TCCTGAAATGGCTTATATTTAAGT | SEQ ID NO: 6323 |
| rs2071481 | CAGACAGGTTTCTCCTGAAACTCTTTCA | SEQ ID NO: 1592 | CAGACAGGTTTCTCCTGAAACTCTTTCG | SEQ ID NO: 3958 | TATACGCCATGTACTGTTCATATC | SEQ ID NO: 6324 |
| rs2071544 | CCGTCCATCGGCTTCTCATTTT | SEQ ID NO: 1593 | CCGTCCATCGGCTTCTCATTTC | SEQ ID NO: 3959 | TCCTATTCAACCCTGAGAGCT | SEQ ID NO: 6325 |
| rs362512 | TCTAGAATATTGCTTCAAAAATGCCAA | SEQ ID NO: 1594 | TCTAGAATATTGCTTCAAAAATGCCAG | SEQ ID NO: 3960 | CAAACTCAGTCAGAAAGCTAGTG | SEQ ID NO: 6326 |
| rs1056032 | TAAGCATGGTCAATTCCCCCTTCT | SEQ ID NO: 1595 | TAAGCATGGTCAATTCCCCCTTCC | SEQ ID NO: 3961 | CCAAAGGAAATGGAGAAAAG | SEQ ID NO: 6327 |
| rs4711185 | CATCCCAAACCCTTAATTATATGCATCT | SEQ ID NO: 1596 | CATCCCAAACCCTTAATTATATGCATCC | SEQ ID NO: 3962 | TTACAATGTCCTTCAGCTATGTT | SEQ ID NO: 6328 |
| rs6457204 | CAGCCCTTAGCAACCACTAATCTGTTA | SEQ ID NO: 1597 | CAGCCCTTAGCAACCACTAATCTGTTT | SEQ ID NO: 3963 | TGCCTTTACGGATTTGTCTAT | SEQ ID NO: 6329 |
| rs7451190 | CATGATTAGGTCTTTTCAACACTGCATA | SEQ ID NO: 1598 | CATGATTAGGTCTTTTCAACACTGCATG | SEQ ID NO: 3964 | TCCCAAACAATACTGTTTTACAA | SEQ ID NO: 6330 |
| rs474534 | GATACGGGTGGGAGCTGCAACA | SEQ ID NO: 1599 | GATACGGGTGGGAGCTGCAACG | SEQ ID NO: 3965 | CGTTCCCTTACCCTGACATAT | SEQ ID NO: 6331 |
| rs1480380 | CGCAGGTTCTTGGGGCAGAT | SEQ ID NO: 1600 | CGCAGGTTCTTGGGGCAGAC | SEQ ID NO: 3966 | GTTTTAACTGAAGTCTCCATTTTA | SEQ ID NO: 6332 |
| rs6914285 | TGTCAGAGGGACACAGGAGTCAA | SEQ ID NO: 1601 | TGTCAGAGGGACACAGGAGTCAG | SEQ ID NO: 3967 | CTGAAAGAGCTCCCTATGGAC | SEQ ID NO: 6333 |
| rs3129953 | AAACAAGCAGTGTTTTCCACAAAAAT | SEQ ID NO: 1602 | AAACAAGCAGTGTTTTCCACAAAAAC | SEQ ID NO: 3968 | TCATCCTGGACTCTAAAATGG | SEQ ID NO: 6334 |
| rs6927487 | GTAACCCAGACTGTGGCAACCAT | SEQ ID NO: 1603 | GTAACCCAGACTGTGGCAACCAC | SEQ ID NO: 3969 | CAACTCTCCATATACCCCAG | SEQ ID NO: 6335 |
| rs3095151 | CCAGGTTTGAGGGAGTGAGTGCT | SEQ ID NO: 1604 | CCAGGTTTGAGGGAGTGAGTGCG | SEQ ID NO: 3970 | GTCCTACAGACAGGGAAATG | SEQ ID NO: 6336 |
| rs1573298 | CCCTATCACCTTTGCCTTATTCCATTG | SEQ ID NO: 1605 | CCCTATCACCTTTGCCTTATTCCATTC | SEQ ID NO: 3971 | TTAGAAGCAAGTCACAGATCC | SEQ ID NO: 6337 |
| rs3104395 | GGAAATGCAACATTTGGACAGGA | SEQ ID NO: 1606 | GGAAATGCAACATTTGGACAGGG | SEQ ID NO: 3972 | ATGCCTGTTCTCACCTAGGT | SEQ ID NO: 6338 |
| rs2395316 | GGAAAGTTTCAACAATAGGATCCAACAA | SEQ ID NO: 1607 | GGAAAGTTTCAACAATAGGATCCAACAG | SEQ ID NO: 3973 | TAGAAGAAAGAACTTCATAGCTCA | SEQ ID NO: 6339 |
| rs1264707 | AGAGAAGCACAAGACAAGAGGCCA | SEQ ID NO: 1608 | AGAGAAGCACAAGACAAGAGGCCG | SEQ ID NO: 3974 | GAATAAGCAAGTCATGCATT | SEQ ID NO: 6340 |
| rs3095313 | TGAGAGTCTAGGGAGGCCCACAA | SEQ ID NO: 1609 | TGAGAGTCTAGGGAGGCCCACAG | SEQ ID NO: 3975 | AGGAAATGATGTTCGGAAAG | SEQ ID NO: 6341 |
| rs2735054 | CAAAAAGCAACATTCAAGATAACCCATA | SEQ ID NO: 1610 | CAAAAAGCAACATTCAAGATAACCCATG | SEQ ID NO: 3976 | CATATGAATGCAACCTTGTACA | SEQ ID NO: 6342 |
| rs866390 | TCTGTCCATAAACATTCTCCATCAT | SEQ ID NO: 1611 | TCTGTCCATATTTCATTCTCCATCAG | SEQ ID NO: 3977 | TCACTTTCCATTTTTTCCTTTT | SEQ ID NO: 6343 |
| rs2523721 | GGAAGCCCTGAATTTTGTCTCTGTCT | SEQ ID NO: 1612 | GGAAGCCCTGAATTTTGTCTCTGTCC | SEQ ID NO: 3978 | TCCTTAGGGTACTCAGGTGGT | SEQ ID NO: 6344 |
| rs2239600 | ACTCCCTTCTTCTCAAGAGATTTCCA | SEQ ID NO: 1613 | ACTCGCTTCTTCTCAAGAGATTTCCG | SEQ ID NO: 3979 | ATCTTCTCATGGTAATTGCTGA | SEQ ID NO: 6345 |
| rs2747466 | TGACTTTAATTCATTCCTGGGCATT | SEQ ID NO: 1614 | TGACTTTAATTCATTCCTGGGCATC | SEQ ID NO: 3980 | TGCCACTATAAAAAGTATTAAACC | SEQ ID NO: 6346 |
| rs1029239 | TCTTGGCGTCTGTGGTGAATAATC | SEQ ID NO: 1615 | TCTTGGCGTCTGTGGTGAATAATG | SEQ ID NO: 3981 | AATTGAGTATCATTTCTTTACTTTTG | SEQ ID NO: 6347 |
| rs2523676 | GCAAAGCTCCTGAGTGGAATTCCT | SEQ ID NO: 1616 | GCAAAGCTCCTGAGTGGAATTCCC | SEQ ID NO: 3982 | CTGCATTATAAGGGTGAGGCT | SEQ ID NO: 6348 |
| rs3757335 | CCAGTGAGGAAAGATCCCACCTTA | SEQ ID NO: 1617 | CCAGTGAGGAAAGATCCCACCTTC | SEQ ID NO: 3983 | GCAGCTACCAAGATTTTTGGT | SEQ ID NO: 6349 |
| rs760070 | CAGCACTTGGGATTGGGGTTAAA | SEQ ID NO: 1616 | CAGCACTTGGGATTGGGGTTAAG | SEQ ID NO: 3984 | CTTTAAAAACGGCTGTCAGGT | SEQ ID NO: 6350 |
| rs2442719 | GAGGCCAGGAGGGCTGGACA | SEQ ID NO: 1619 | GAGGCCAGGAGGGCTGGACG | SEQ ID NO: 3985 | AGAGAAACTGAGGTGAGGTCAG | SEQ ID NO: 6351 |
| rs1573297 | GAACCTGAATTCTGCCAACAACCT | SEQ ID NO: 1620 | GAACCTGAATTCTGCCAACAACCC | SEQ ID NO: 3986 | AGTGAGCTTGGAAGCAGATT | SEQ ID NO: 6352 |
| rs3132569 | ATAATGACATGAGCGAGCGCCAAT | SEQ ID NO: 1621 | ATAATGACATGAGCGAGCGCCAAC | SEQ ID NO: 3987 | TAATAGCCTTGGCAGTAAACG | SEQ ID NO: 6353 |
| rs2844523 | ACCCCGTCTGCTCTACCCCATT | SEQ ID NO: 1622 | ACCCCGTCTGCTCTACCCCATC | SEQ ID NO: 3988 | ACTACGTTTTCTCCCTCAGC | SEQ ID NO: 6354 |
| rs2077573 | AGGCAGGAGGAGAAGCAAGTTGTA | SEQ ID NO: 1623 | AGGCAGGAGGAGAAGCAAGTTGTG | SEQ ID NO: 3989 | GATCAGCTACATCTGGGCTT | SEQ ID NO: 6355 |
| rs2249168 | GAGCTCTCATGAGAAAGCCCAACT | SEQ ID NO: 1624 | GAGCTCTCATGAGAAAGCCCAACC | SEQ ID NO: 3990 | GGATGTGAAAACTTGGAATC | SEQ ID NO: 6356 |
| rs2844780 | GAATCTTGCACCTTAAACTCACACATCA | SEQ ID NO: 1625 | GAATCTTGCACCTTAAACTCACACATCC | SEQ ID NO: 3991 | AATCCACTATCTCTCTCATTCTTC | SEQ ID NO: 6357 |
| rs6457655 | TTAGTATGGCCACTAGGGAAAACATT | SEQ ID NO: 1626 | TTAGTATGGCCACTAGGGAAAACATG | SEQ ID NO: 3992 | TGAAGGTTCTTCAAAAAGTTAAA | SEQ ID NO: 6358 |
| rs7770024 | GAGGAGAGGAGGATGCACAGATGA | SEQ ID NO: 1627 | GAGGAGAGGAGGATGCACAGATGG | SEQ ID NO: 3993 | ACTATAACAGAAAGAGAAGAGAGAGA | SEQ ID NO: 6359 |
| rs458434 | GGGTCACAATTATTTAGGCAAATCACTT | SEQ ID NO: 1628 | GGGTCACAATTATTTAGGCAAATCACTG | SEQ ID NO: 3994 | ATCCCCATAATCAAAGTATTCC | SEQ ID NO: 6360 |
| rs204894 | TCATTTGAAACCGTCCAGATCAAT | SEQ ID NO: 1629 | TCATTTGAAACCGTCCAGATCAAC | SEQ ID NO: 3995 | TTATCCCCACCTCTGATGATT | SEQ ID NO: 6361 |
| rs6905949 | TGCTGGACTTGCACTAATTTGTACAGTT | SEQ ID NO: 1630 | TGCTGGAGTTGCACTAATTTGTACAGTC | SEQ ID NO: 3996 | ACTTCGTACTTTGACACACACC | SEQ ID NO: 6362 |
| rs3135376 | TGTTATTCTCGTGTGATGAGAATCTCAA | SEQ ID NO: 1631 | TGTTATTCTCGTGTGATGAGAATCTCAG | SEQ ID NO: 3997 | ACAGTGAGGTTTAGTAACAAGTCA | SEQ ID NO: 6363 |
| rs1018434 | TTGGGACTTGTGAGGTATCACTCAT | SEQ ID NO: 1632 | TTGGGACTTGTGAGGTATCACTCAC | SEQ ID NO: 3998 | TGGTGCTGGATTTTCTCATAG | SEQ ID NO: 6364 |
| rs204879 | AACTCCACCACAAAACATACTGCATT | SEQ ID NO: 1633 | AACTCCACCACAAAACATACTGCATC | SEQ ID NO: 3999 | CTCACTGTCTGCAGACATCTG | SEQ ID NO: 6365 |
| rs3129943 | CCATAAAACTGCCTGTGAAAGTAAAAA | SEQ ID NO: 1634 | CCATAAAACTGCCTGTGAAAGTAAAAG | SEQ ID NO: 4000 | TCTGAAGAATATTGAGCTCTGAA | SEQ ID NO: 6366 |
| rs7772569 | TCCTGCTTCATCCAGCCAGAATAT | SEQ ID NO: 1635 | TCCTGCTTCATCCAGCCAGAATAC | SEQ ID NO: 4001 | GCGAACCTTGTTCTATTTGCT | SEQ ID NO: 6367 |
| rs2248902 | TGGTCCATAGATTCATTTCTCACTCACT | SEQ ID NO: 1636 | TGGTCCATAGATTCATTTCTCACTCACC | SEQ ID NO: 4002 | CTTGGAGAAAACTATGGAAAAC | SEQ ID NO: 6368 |
| rs2517449 | TGTGTCATCTTCTCATGGGCCAA | SEQ ID NO: 1637 | TGTGTCATCTTCTCATGGGCCAG | SEQ ID NO: 4003 | GTGGTCCTTTTTGCATGTTTTA | SEQ ID NO: 6369 |
| rs2659448 | AGTGTCTCATGGAGAGGCCCTTA | SEQ ID NO: 1638 | AGTGTCTCATGGAGAGGCCCTTG | SEQ ID NO: 4004 | TTCCTGTAGCGAAAGGGTAGATA | SEQ ID NO: 6370 |
| rs2857694 | GGCGGGGCCTTCCAGTTATTAA | SEQ ID NO: 1639 | GGCGGGGCCTTCCAGTTATTAT | SEQ ID NO: 4005 | TTACGTCATGCAAATGAGGT | SEQ ID NO: 6371 |
| rs3132671 | CCTCCTCCTGCAGCTTTGCATAT | SEQ ID NO: 1640 | CCTCCTCCTGCAGCTTTGCATAC | SEQ ID NO: 4006 | CTGTATTTCTACAATAGCACTATCAC | SEQ ID NO: 6372 |
| rs2395301 | GCACTGTCCTACTCAGCAGCGAATA | SEQ ID NO: 1641 | GCACTGTCCTACTCAGCAGCGAATG | SEQ ID NO: 4007 | AGACCAGCTTGTGAGACATG | SEQ ID NO: 6373 |
| rs2516455 | GGAAAAATCTACTGGCAATGGAATCTT | SEQ ID NO: 1642 | GGAAAAATCTACTGGCAATGGAATCTG | SEQ ID NO: 4008 | CAGTTAGTTGTTAATTAACTGGAAA | SEQ ID NO: 6374 |
| rs2294472 | GCAAGCAGGTAATGGGAAAGTTTTTAA | SEQ ID NO: 1643 | GCAAGCAGGTAATGGGAAAGTTTTTAG | SEQ ID NO: 4009 | AATGCAAGGGAATAACACAGA | SEQ ID NO: 6375 |
| rs2077580 | CCCACTCATGGCCACAGCTTT | SEQ ID NO: 1644 | CCACTCATGGCCACAGCTTC | SEQ ID NO: 4010 | CTCTCCTTCCTCACAAGACC | SEQ ID NO: 6376 |
| rs1061783 | GGGGAACCACAGTTTCACCATCA | SEQ ID NO: 1645 | GGGGAACCACAGTTTCACCATCG | SEQ ID NO: 4011 | CCGCCCATtCTACTACTCAAC | SEQ ID NO: 6377 |
| rs3135024 | TTCAGCTCCTTGAGGCTGTGGA | SEQ ID NO: 1646 | TTCAGCTCCTTGAGGCTGTGGG | SEQ ID NO: 4012 | CAGAAACTTCTGGAAGCCACT | SEQ ID NO: 6378 |
| rs204994 | GTGGATCCCACTGTCACCCCA | SEQ ID NO: 1647 | GTGGATCCCACTGTCACCCCG | SEQ ID NO: 4013 | CTGACTGTTTTGCACACTTC | SEQ ID NO: 6379 |
| rs1059174 | CACCTAAACTGAGTCAAGCTGGCTAA | SEQ ID NO: 1648 | CACCTAAACTGAGTCAAGCTGGCTAG | SEQ ID NO: 4014 | TCCAAATATATGTATATCTTTTCACC | SEQ ID NO: 6380 |
| rs1233422 | CGTGATGTTCACTGTCTGCCAAGA | SEQ ID NO: 1649 | CGTGATGTTCACTGTCTGCCAAGG | SEQ ID NO: 4015 | TGATCAGCTGTAGGCATCAC | SEQ ID NO: 6381 |
| rs7772297 | GCTGGAAGTCATCGTCAGCAAAG | SEQ ID NO: 1650 | GCTGGAAGTCATCGTCAGCAAAC | SEQ ID NO: 4016 | AACATGGGAACAGATAACCTAA | SEQ ID NO: 6382 |
| rs3132613 | GATCATCTCCCCTGCTGGAAACG | SEQ ID NO: 1651 | GATCATCTCCCCTGCTGGAAACC | SEQ ID NO: 4017 | AATCAGAGATCTTTATTTTATTCAGT | SEQ ID NO: 6383 |
| rs2272592 | TCCATGTCGCCCACATTTTAAAT | SEQ ID NO: 1652 | TCCATGTCGCCCACACTTTAAAC | SEQ ID NO: 4018 | CCCATTGCGTAAAAACACTT | SEQ ID NO: 6384 |
| rs2523790 | GGAGAAGGTCCCTCCAGGAGAA | SEQ ID NO: 1653 | GGAGAAGGTCCCTCCAGGAGAC | SEQ ID NO: 4019 | TTCATGATGAGGAAAAACCC | SEQ ID NO: 6385 |
| rs2071476 | CATGAGACGGTTATTTTGAGAACCACA | SEQ ID NO: 1654 | CATGAGACGGTTATTTTGAGAACCACG | SEQ ID NO: 4020 | TCTATATGTGAACACAGTTTAAAAGC | SEQ ID NO: 6386 |
| rs2105903 | GCTTTTACACCATTGAAAAGTCAAAAAA | SEQ ID NO: 1655 | GCTTTTACACCATTGAAAAGTCAAAATAT | SEQ ID NO: 4021 | CCTAAGTCAAACCATCTTAAGTCA | SEQ ID NO: 6387 |
| rs7382555 | TGACAATAATTTGTTTGGCTAGGGTAA | SEQ ID NO: 1656 | TGACAATAATTTGTTTGGCTAGGGTAG | SEQ ID NO: 4022 | CAGAAATTGTTTACTCCAATCTAG | SEQ ID NO: 6388 |
| rs3115663 | AAACACAGTGAAAGAAACAGGAAAGAA | SEQ ID NO: 1657 | AAACACAGTGAAAGAAACAGGAAAGAG | SEQ ID NO: 4023 | ACAGAGAAAACATAACCCAGAAG | SEQ ID NO: 6389 |
| rs492899 | CAGGGCTGGGGGTGTGTGTAT | SEQ ID NO: 1658 | CAGGGCTGGGGGTGTGTGTAC | SEQ ID NO: 4024 | TAGAGCCTTTGCTGATCCTTT | SEQ ID NO: 6390 |
| rs707939 | GAGAGTGGGTGTAGCCTTCAGATGTA | SEQ ID NO: 1659 | GAGAGTGGGTGTAGCCTTCAGATGTC | SEQ ID NO: 4025 | TTTGGGGGAGATATTAGGCTTA | SEQ ID NO: 6391 |
| rs3094614 | CTCCTGCCGAACTTCGCCA | SEQ ID NO: 1660 | CTCCTGCGGAACTTCGOCG | SEQ ID NO: 4026 | CCAATGGGAACCTTAGTCTCT | SEQ ID NO: 6392 |
| rs2734990 | GCTAAGTACTTCCAGGAAACTCATTCAT | SEQ ID NO: 1661 | GCTAAGTACTTCCAGGAAACTCATTCAA | SEQ ID NO: 4027 | TGGTAAACATCATTAAGCACCTA | SEQ ID NO: 6393 |
| rs2273612 | ACCTAGGTCTTCCTGGGAGGGTA | SEQ ID NO: 1662 | ACCTAGCTCTTCCTGGGAGGGTG | SEQ ID NO: 4028 | CATAGGACCCAGAGTGAGGAG | SEQ ID NO: 6394 |
| rs438999 | AGTCACCTGGCCAGAAGGGATACT | SEQ ID NO: 1663 | AGTCACCTGGCCAGAAGGGATACC | SEQ ID NO: 4029 | AGTTGGAAACCCCATAAGG | SEQ ID NO: 6395 |
| rs206786 | GGGATGGGGAAAGAGAATGTGACA | SEQ ID NO: 1664 | GGGATGGGGAAAGAGAATGTGACC | SEQ ID NO: 4030 | AGAATTGAGATAGTAAGGATAATTTCA | SEQ ID NO: 6396 |
| rs362536 | GAAATGTCTGCAATATCTAGATTGGGAA | SEQ ID NO: 1665 | GAAATGTCTGCAATATCTAGATTGGGAG | SEQ ID NO: 4031 | ATATTTCTGAATGGAGTATGAGG | SEQ ID NO: 6397 |
| rs2523395 | GAGAGTGATGATGGCTACATAACATTGT | SEQ ID NO: 1666 | GAGAGTGATGATGGCTACATAACATTGC | SEQ ID NO: 4032 | AACCTACTAATTGTCACTGCATT | SEQ ID NO: 6398 |
| rs1064190 | ACCAGCCCTCCTCTGTGGGACT | SEQ ID NO: 1667 | ACCAGCCCTCCTCTGTGGGACG | SEQ ID NO: 4033 | AGAACTATCCTGTGGTCCTGC | SEQ ID NO: 6399 |
| rs3130267 | GGATGTTGTGCGCTGAGGGA | SEQ ID NO: 1668 | GGATGTTGTGCGCTGAGGGC | SEQ ID NO: 4034 | CTTTTTGGTGGTCTTGGTCTT | SEQ ID NO: 6400 |
| rs7762933 | AGGAAAGCGGAATGAAAGCAGGT | SEQ ID NO: 1669 | AGGAAAGCGGAATGAAAGCAGGC | SEQ ID NO: 4035 | AAAAGTGGCTGGGAGAAGAT | SEQ ID NO: 6401 |
| rs3094211 | CCCAGGCCCAACTTACCCCA | SEQ ID NO: 1670 | CGCAGGCCCAACTTACCCCG | SEQ ID NO: 4036 | GGTTCCTCCATGACTCTTTC | SEQ ID NO: 6402 |
| rs3129932 | TCTTCACATCCTGAAGTTGGTACTCTTC | SEQ ID NO: 1671 | TCTTCACATCCTGAAGTTGGTACTCTTG | SEQ ID NO: 4037 | CAATCATAATTACCCTAACTGTGT | SEQ ID NO: 6403 |
| rs2275855 | AAACCAAGGGCAGGATCAGGAA | SEQ ID NO: 1672 | AAACCAAGGGCAGGATCAGGAG | SEQ ID NO: 4038 | AACAAGGAAAGCAGATGTGG | SEQ ID NO: 6404 |
| rs2516399 | TTTTAATTCCAGGAAATAAATGCCCA | SEQ ID NO: 1673 | TTTTAATTCCAGGAAATAAATGCCCG | SEQ ID NO: 4039 | GACTCAGATATAAAAAGGAGAATCT | SEQ ID NO: 6405 |
| rs2857210 | AGAAGAGTAGAGTGGGCCCTTATGTACT | SEQ ID NO: 1674 | AGAAGAGTAGAGTGGGCCCTTATGTACC | SEQ ID NO: 4040 | TGAAGAAGTGAAGAATATAACCTG | SEQ ID NO: 6406 |
| rs7357044 | TGGAAATTAAATTTTTAGCCACTCAAT | SEQ ID NO: 1675 | TGGAAATTAAATTTTTAGCCACTCAAC | SEQ ID NO: 4041 | GAAAGTATATTAGCTTTGGGAGTG | SEQ ID NO: 6407 |
| rs592229 | CAGTGGCTCGTCTCCACCCA | SEQ ID NO: 1676 | CAGTGGCTCGTCTCCACCCC | SEQ ID NO: 4042 | TCCTCAGACAGCACACTCTC | SEQ ID NO: 6408 |
| rs2844773 | TGAAACCATCAAGGCAATGTGATAT | SEQ ID NO: 1677 | TGAAACCATCAAGGCAATGTGATAG | SEQ ID NO: 4043 | CTTACTTCCATAAAAGATATAAAACC | SEQ ID NO: 6409 |
| rs2523459 | GGTGGTGGGTTTTAATGAACATTTTCT | SEQ ID NO: 1678 | GGTGGTGGGTTTTAATGAACATTTTCG | SEQ ID NO: 4044 | TTTTAAAACTCTGTAACTATTTCGTT | SEQ ID NO: 6410 |
| rs2734962 | ACATATCGTTTTACCACAATTTCACCA | SEQ ID NO: 1679 | ACATATCGTTTTACCACAATTTCACCG | SEQ ID NO: 4045 | TTTCCTATTGATGTACATGTGG | SEQ ID NO: 6411 |
| rs2894046 | CCTGCTGCCTCCCCAAACTCT | SEQ ID NO: 1680 | CCTGCTGCCTCCCCAAACTCC | SEQ ID NO: 4046 | TCGTCCTCTTCTCCTTCAAC | SEQ ID NO: 6412 |
| rs15574 | GGGGTACATATGGGAGCCTGGA | SEQ ID NO: 1681 | GGGGTACATATGGGAGCCTGGG | SEQ ID NO: 4047 | TTGGGGAGTCAGCTCTGTAC | SEQ ID NO: 6413 |
| rs6919855 | ACATGCACAGTATTTGAAATCTGTGTA | SEQ ID NO: 1682 | ACATGCACAGTATTTGAAATCTGTGTG | SEQ ID NO: 4048 | ATTTACACATGCATACGTATGTTT | SEQ ID NO: 6414 |
| rs1362076 | CCAGCTCCATCCCGTCAATGA | SEQ ID NO: 1683 | CCAGCTCCATCCCGTCAATGC | SEQ ID NO: 4049 | CTATAGCCATCACAGCAGCAC | SEQ ID NO: 6415 |
| rs740864 | GCCCTGATGGCCACTGAGCT | SEQ ID NO: 1684 | GCCCTGATGGCCACTGAGCC | SEQ ID NO: 4050 | TGCTCATTCTCCTGACCATAG | SEQ ID NO: 6416 |
| rs6922512 | GTTTGGATACAAAGGACAGGAGACTAT | SEQ ID NO: 1685 | GTTTGGATACAAAGGACAGGAGACTAC | SEQ ID NO: 4051 | GGATGTCTGTTCTAAAAGAGAGTG | SEQ ID NO: 6417 |
| rs6926737 | GAACTTTATGAAATTATTCCTGCCTCAA | SEQ ID NO: 1686 | GAACTTTATGAAATTATTCCTGCCTCAG | SEQ ID NO: 4052 | AACCCTAATTTCATGAGACAATAT | SEQ ID NO: 6418 |
| rs2523747 | AATTCAGGCACTGCATTCCTCA | SEQ ID NO: 1687 | ATTCAGGCACTTTGCATTCCTCG | SEQ ID NO: 4053 | AGGACACTCCAGCAGCATAC | SEQ ID NO: 6419 |
| rs2023470 | TGGCTACAAGGAATGGGCAGTT | SEQ ID NO: 1688 | TGGCTTTACAAGGAATGGGCAGTG | SEQ ID NO: 4054 | TTTGCACATGTGCCATATTC | SEQ ID NO: 6420 |
| rs2239839 | GAGTCCAGGTTGACTGATGGGGT | SEQ ID NO: 1689 | GAGTCCAGGTTGACTGATGGGGG | SEQ ID NO: 4055 | GCATGAGAAGGAAAGCAAGA | SEQ ID NO: 6421 |
| rs3131050 | AGTAGGAAGGAATCTATGTCGGACTCA | SEQ ID NO: 1690 | AGTAGGAAGGAATCTATGTCGGACTCG | SEQ ID NO: 4056 | GCTGTTAGTAAGTTGTCTTTAATGG | SEQ ID NO: 6422 |
| rs2523984 | CTAGGGGCCAGCAGTTTAGGAGTA | SEQ ID NO: 1691 | CTAGGGGCCAGCAGTTTAGGAGTG | SEQ ID NO: 4057 | AAGTAAAACAATTTACAGATGTTAGA | SEQ ID NO: 6423 |
| rs3132965 | TGGGGTTGAAAAGAGGAGACCCA | SEQ ID NO: 1692 | TGGGGTTGAAAAGAGGAGACCCG | SEQ ID NO: 4058 | TAAGAGGTGGCTGAAGGAAAT | SEQ ID NO: 6424 |
| rs3115537 | CCTGCCTATTGCCTGCGG | SEQ ID NO: 1693 | CCTGCCAATTGCCTGCGC | SEQ ID NO: 4059 | TTGTGGTCTCTTCCACTTTCT | SEQ ID NO: 6425 |
| rs2647044 | TCATTATCCCCAGTTCAATGACTTTAAT | SEQ ID NO: 1694 | TCATTATCCCCAGTTCAATGACTTTAAC | SEQ ID NO: 4060 | GTAATATGCGTATGTTTTTGTATGT | SEQ ID NO: 6426 |
| rs1419640 | TGGTCAGGATATAAAATGGTACAACTGCT | SEQ ID NO: 1695 | TGGTCAGGATATAAAATGGTACAACTGCG | SEQ ID NO: 4061 | TGGAGAGAGTATAGTCGTTTCTT | SEQ ID NO: 6427 |
| rs8283 | ATTCCTGTGAACCCCCAGTCCT | SEQ ID NO: 1696 | ATTCCTGTGAACCCCCAGTCCC | SEQ ID NO: 4062 | GCTTCTTTGTTTGAGGGGAT | SEQ ID NO: 6428 |
| rs2517810 | CCCGCTCCTGAGTArCTCCCCG | SEQ ID NO: 1697 | CCCGCTCCTGAGTATCTCCCCC | SEQ ID NO: 4063 | TTCTTCAGAGGACTTCCCCT | SEQ ID NO: 6429 |
| rs2394694 | AACCCTGTGATCTTCAATGAGCCT | SEQ ID NO: 1698 | AACCCTGTGATCTTCAATGAGCCC | SEQ ID NO: 4064 | ACAAAAATAAGCAATGGGCA | SEQ ID NO: 6430 |
| rs2076483 | CCTCTCTTTCCCTCCTCCCCACT | SEQ ID NO: 1699 | CCTCTCTTTCCCTCCTCCCCACC | SEQ ID NO: 4065 | CTTCATCACCTCCTCTTCTCC | SEQ ID NO: 6431 |
| rs411337 | ATGGCAGGAAGGGTGGGGGA | SEQ ID NO: 1700 | ATGGCAGGAAGGGTGGGGGG | SEQ ID NO: 4066 | GTCTAGTTGGGCAAAAATTAG | SEQ ID NO: 6432 |
| rs6934993 | CCGGTAAGAATTTAGCAAGTGATGAAAT | SEQ ID NO: 1701 | CCGGTAAGAATTTAGCAAGTGATGAAAC | SEQ ID NO: 4067 | GGATAATGCCTAGTAGGTAATTAA | SEQ ID NO: 6433 |
| rs1245371 | CTTGTCCCTCCCCACCCCCA | SEQ ID NO: 1702 | CTTGTCCCTCCCCACCCCCG | SEQ ID NO: 4068 | ATCCTGCAAAAGAGGTACAAAG | SEQ ID NO: 6434 |
| rs6902723 | CCTCCTCCACTGTTATGTGTAACTATCAA | SEQ ID NO: 1703 | CCTCCTCCACTGTTATGTGTAACTATCAG | SEQ ID NO: 4069 | CTAATAGGAGGAACAAGAAAAAAA | SEQ ID NO: 6435 |
| rs3134942 | CGGGACAAATCCACACCCATT | SEQ ID NO: 1704 | CGGGACAAATCCACACCCATG | SEQ ID NO: 4070 | CCACCACAAACCTGTAGAGG | SEQ ID NO: 6436 |
| rs454212 | GAGCTAGTGGTTTGTCCAAGGCT | SEQ ID NO: 1705 | GAGCAAGTGGTTTGTCCAAGGCC | SEQ ID NO: 4071 | CATGGTTGCAGAGCTAGGAC | SEQ ID NO: 6437 |
| rs1362126 | AGGCGGTGTCTGGGGTTGGA | SEQ ID NO: 1706 | AGGCGGTGTCTGGGGTTGGG | SEQ ID NO: 4072 | GGCTCAGTATTGAGAATTCCC | SEQ ID NO: 6438 |
| rs2075701 | AGCACCTGCAAGAAAAAGGGTGA | SEQ ID NO: 1707 | AGCACCTGOAAGAAAAAGGGTGG | SEQ ID NO: 4073 | CATTGGACACACTGCTGTC | SEQ ID NO: 6439 |
| rs3130502 | CCATATGAGGCTGCCGGCA | SEQ ID NO: 1708 | CCATATGAGGCTGCCGGGG | SEQ ID NO: 4074 | ACAGAGGTGCACAGCTAGTG | SEQ ID NO: 6440 |
| rs1057373 | ACTCATCCTGGAGGCAGCTGCA | SEQ ID NO: 1709 | ACTCATCCTGGAGGCAGCTGCC | SEQ ID NO: 4075 | ACTCTGCAGCTGTGGTTCTC | SEQ ID NO: 6441 |
| rs1736936 | CCAOCACCCTCTCACTCCTGAAT | SEQ ID NO: 1710 | CCACCACCCTCTCACTCCTGAAC | SEQ ID NO: 4076 | GTGGCTCTAGCACTGTTCCAT | SEQ ID NO: 6442 |
| rs7763159 | CCCCAGTCCAAAGTGTTTTCTGCT | SEQ ID NO: 1711 | CCCCAGTCCAAAGTGTTTTCTGCC | SEQ ID NO: 4077 | CTTCACACAGTTATGATACTACTAACA | SEQ ID NO: 6443 |
| rs2517755 | AGGACCCACACCTGCTTTCCTT | SEQ ID NO: 1712 | AGGACCCACACCTGCTTTCCTC | SEQ ID NO: 4078 | TGTTTCCTGATCCTGTCCT | SEQ ID NO: 6444 |
| rs3131000 | ACACAGCAGAGCTGCCTGCAG | SEQ ID NO: 1713 | ACACAGCAGAGOTGCCTGCAC | SEQ ID NO: 4079 | ACTGTGGACGTCACCAAACT | SEQ ID NO: 6445 |
| rs1233364 | GGGAAATGCAACAGAGAATTAAATAAAGT | SEQ ID NO: 1714 | GGGAAATGCAACAGAGAATTAAATAAAGC | SEQ ID NO: 4080 | GTCTAAAAGAATCTGCTTTGTTG | SEQ ID NO: 6446 |
| rs2239804 | GAGCATCAAACTCTGGGGGGAA | SEQ ID NO: 1715 | GAGCATCAAACTCTGGGGGGAG | SEQ ID NO: 4061 | TAAGGCAGAGTACATGGTAATGA | SEQ ID NO: 6447 |
| rs3132668 | GGCAAACAGCACACCAGAACCA | SEQ ID NO: 1716 | GGCAAACAGCACACCAGAACCT | SEQ ID NO: 4082 | GAAGCAAAATCGTTTTTCC | SEQ ID NO: 6448 |
| rs2074464 | ACCTGCTCCTCCCACCTGATCA | SEQ ID NO: 1717 | ACCTGCTCCTCCCACCTGATCG | SEQ ID NO: 4083 | GGTCTCCCTCTTCrATGGC | SEQ ID NO: 6449 |
| rs2857107 | GAAAACAAAGGCCGCAGATTATAAT | SEQ ID NO: 1718 | GAAAACAAAGGCCGCAGARRAAAAC | SEQ ID NO: 4084 | GTCTTTCAAGAATTTTGAAAGTG | SEQ ID NO: 6450 |
| rs1362063 | GAATGTTGTTTAGCCAACTTCTCTGTAT | SEQ ID NO: 1719 | GAATGTTGTTTAGCCAACTTCTCTGTAC | SEQ ID NO: 4085 | AAAATATTTCTTTCACTGCAATTT | SEQ ID NO: 6451 |
| rs1536215 | CCAATCTCTGTTACTCTCACGTAATCCTC | SEQ ID NO: 1720 | CCAATCTCTGTTACTCTCACGTAATCCTG | SEQ ID NO: 4086 | ACATACCAGAAAACCAAGTTATATG | SEQ ID NO: 6452 |
| rs3135395 | TGGTGATGTCAAATGGTGACGAGA | SEQ ID NO: 1721 | TGGTGATGTCAAATGGTGACGAGC | SEQ ID NO: 4087 | ACTATAGCCATGAGAATTCAGTC | SEQ ID NO: 6453 |
| rs3757328 | GGGGCTTCCTCTCCTTCGACTT | SEQ ID NO: 1722 | GGGGCTTCCTCTCCTTCGACTC | SEQ ID NO: 4088 | TGCCAGATAAGTGAGGCTAG | SEQ ID NO: 6454 |
| rs3130982 | CAGCAGCTCCAGTTCCCAATCG | SEQ ID NO: 1723 | CAGCAGCTCCAGTTCCCAATCC | SEQ ID NO: 4089 | GTGGCAAAATCATCCTTCAG | SEQ ID NO: 6455 |
| rs2395269 | GCCATAAATTCTTGCACCTGGGA | SEQ ID NO: 1724 | GCCATAAATTCTTGCACCTGGGC | SEQ ID NO: 4090 | TGCTTCATGCTGGTACCTT | SEQ ID NO: 6456 |
| rs1264423 | TCATATGAGAATGTCTCTGTTCTGTCAA | SEQ ID NO: 1725 | TCATATGAGAATGTCTCTGTTCTGTCAG | SEQ ID NO: 4091 | CCTGATTTTTTTTGTTCTTCTTT | SEQ ID NO: 6457 |
| rs2532929 | GATGGAAACCTGGTCCCAACAT | SEQ ID NO: 1726 | GATGGAAACCTGGTCCCAACAC | SEQ ID NO: 4092 | TAACTCCTAGTCCCCTGGG | SEQ ID NO: 6458 |
| rs805303 | GTTAGGGCTTCACTGGCCACATT | SEQ ID NO: 1727 | GTTAGGGCTTCACTGGCCACATC | SEQ ID NO: 4093 | ACAGGGTTTGGCCTTCATAAT | SEQ ID NO: 6459 |
| rs2260000 | TATCAATCATCCTCCCACCCTCCCT | SEQ ID NO: 1728 | TATCAATCATCCTCCCACCCTCCCC | SEQ ID NO: 4094 | CTATCTCATAGGTCAAAGACTAAGTC | SEQ ID NO: 6460 |
| rs86567 | CATGCACCCAGCACAATGGT | SEQ ID NO: 1729 | CATGCACCCAGCACAATGGG | SEQ ID NO: 4095 | GGCCAGGGAAATAAAAGAAA | SEQ ID NO: 6461 |
| rs3117328 | GATGGACTGTTTCCTGGCTCTATATACA | SEQ ID NO: 1730 | GATGGACTGTTTCCTGGCTCTATATACC | SEQ ID NO: 4096 | ATATCTAACAGTCCCATAGAAGATG | SEQ ID NO: 6462 |
| rs4711203 | TCCAATCACCTCCCATGAGGTT | SEQ ID NO: 1731 | TCCAATCACCTCCCATGAGGTC | SEQ ID NO: 4097 | CTCCTTCAATACTGGGGATTAC | SEQ ID NO: 6463 |
| rs2763982 | AAAATTTTGTTTCTTGTCCCTTTCTTC | SEQ ID NO: 1732 | AAAATTTTGTTTCTTGTCCCTTTCTTG | SEQ ID NO: 4098 | TACTTTTCCTTTCTAGGAATGTAGA | SEQ ID NO: 6464 |
| rs3130349 | AGGGCCAGAGACCAGCTCCA | SEQ ID NO: 1733 | AGGGCCAGAGACCAGCTCCG | SEQ ID NO: 4099 | AGAGCAGAGGGGTGAGTCTT | SEQ ID NO: 6465 |
| rs2021365 | TGTTACCAATGGCAGAATTTCCTTCTT | SEQ ID NO: 1734 | TGTTACCAATGGCAGAATTTCCTTCTC | SEQ ID NO: 4100 | TTAAGGCTGAATAATATTGCATT | SEQ ID NO: 6466 |
| rs3117231 | GCAGCCCTCTGTGACCACAAGT | SEQ ID NO: 1735 | GCAGCCCTCTGTGACCACAAGC | SEQ ID NO: 4101 | CAAGTTGCACTCCCTTCTG | SEQ ID NO: 6467 |
| rs2021719 | CGCCCTCCAGGATCCAATAGGA | SEQ ID NO: 1736 | CGCCCTCCAGGATCCAATAGGG | SEQ ID NO: 4102 | ATAGGTTCAGATGCCTGGGT | SEQ ID NO: 6468 |
| rs5009557 | GCAACCATTGATACTAATTGCTTAGGTT | SEQ ID NO: 1737 | GCAACCATTGATACTAATTGCTTAGGTC | SEQ ID NO: 4103 | GTTAATTTATTGGGGATTGCT | SEQ ID NO: 6469 |
| rs1269556 | TGGGTCTCCTTGTTATCTTCTTTCAT | SEQ ID NO: 1738 | TGGGTCTCCTTGTTATCTTCTTTCAC | SEQ ID NO: 4104 | ATATTATGAAATTTTCTCTCAGAATT | SEQ ID NO: 6470 |
| rs3131785 | GGAAGGTCAATACCTGAGCCGTG | SEQ ID NO: 1739 | GGAAGGTCAATACCTGAGOCGTC | SEQ ID NO: 4105 | CTCACCTAGATGGCTGCAG | SEQ ID NO: 6471 |
| rs362521 | CCTCTGCCTTCTTCACACCCATTAT | SEQ ID NO: 1740 | CCTCTGCCTTCTTCACACCCATTAC | SEQ ID NO: 4106 | TTGTGGGAATGGATGAAAG | SEQ ID NO: 6472 |
| rs644774 | CTGACCTAGGTCTGTCTGCCCCA | SEQ ID NO: 1741 | CTGACCTAGGTCTGTCTGCCCCG | SEQ ID NO: 4107 | CCTCTGCAGTGATCGTGTT | SEQ ID NO: 6473 |
| rs558702 | GCCCCGTGGCTAACAGAGCTA | SEQ ID NO: 1742 | GCCCCGTGGCTAACAGAGCTG | SEQ ID NO: 4108 | TGGTAGCTATTACCCACGC | SEQ ID NO: 6474 |
| rs3132571 | AGTGCTGGAGGCTGACTGGCA | SEQ ID NO: 1743 | AGTGCTGGAGGCTGACTGGCG | SEQ ID NO: 4109 | GTGGAAGCACTCAACTCAGC | SEQ ID NO: 6475 |
| rs6932290 | CGGAGTGCAAGGGTGGAAACT | SEQ ID NO: 1744 | CGGAGTGCAAGGGTGGAAACC | SEQ ID NO: 4110 | GCTTTGGGAAAACTACCGT | SEQ ID NO: 6476 |
| rs429608 | GGTGGAGACGAGCCACTGGA | SEQ ID NO: 1745 | GGTGGAGACGAGCCACTGGG | SEQ ID NO: 4111 | AGTCAATCCTTGGCCTCTTC | SEQ ID NO: 6477 |
| rs2245738 | TGGGGCCCACAAGGAATTATCTT | SEQ ID NO: 1746 | TGGGGCCCACAAGGAATTATCTG | SEQ ID NO: 4112 | TGAGCAATTTGTGAGGGAG | SEQ ID NO: 6478 |
| rs1633034 | ATCAGGTAGTCTGAGGCCGCCT | SEQ ID NO: 1747 | ATCAGGTAGTCTGAGGCCGCCC | SEQ ID NO: 4113 | CTTTGTTCTTTTTGTGTAGGATTG | SEQ ID NO: 6479 |
| rs2524179 | TTTCTTCTGAGTTTTCACTAGCTGCAA | SEQ ID NO: 1748 | TTTCTTCTGAGTTTTCACTAGCTGCAG | SEQ ID NO: 4114 | TTTTCGTCAGAGACTTCATG | SEQ ID NO: 6480 |
| rs7776164 | TGAAAACATCTGACCCTTGAGTTTTA | SEQ ID NO: 1749 | TGAAAACATCTGACCCTTGAGTTTTC | SEQ ID NO: 4115 | TTCTGGGAATATATATTATAGGTTCT | SEQ ID NO: 6481 |
| rs3095226 | GAGTTTTGGTGGGGACATTCA | SEQ ID NO: 1750 | GAGTTTTGGTGGGGACATTCC | SEQ ID NO: 4116 | CTTTAGCACTAGGTATTCTGGTTT | SEQ ID NO: 6482 |
| rs6457044 | CATTGTGAGTCCATTGCTTGCAC | SEQ ID NO: 1751 | CATTGTGAGTCCATTGCTTGCAG | SEQ ID NO: 4117 | TCAGGGTTCAAGATATAGAGTCA | SEQ ID NO: 6483 |
| rs362515 | GAGCCTGGAGCAAGTGCCTAAGT | SEQ ID NO: 1752 | GAGCCTGGAGCAAGTGCCTAAGC | SEQ ID NO: 4118 | CCCAGGAACTAAGAAGAGTAAAA | SEQ ID NO: 6484 |
| rs3131934 | TCCACCATGGTCTCAAAGGCTACA | SEQ ID NO: 1753 | TCCACCATGGTCTCAAAGGCTACG | SEQ ID NO: 4119 | GCATAGTCCTTCCTGAAAAATAT | SEQ ID NO: 6485 |
| rs750332 | CTTGTGTGCTTGCTGGGATCATA | SEQ ID NO: 1754 | CTTGTGTGCTTGCTGGGATCATG | SEQ ID NO: 4120 | CAACCTGCTTACTTTTTCTCTTT | SEQ ID NO: 6486 |
| rs5009448 | CTGCCAAGGCAGGGTGGCT | SEQ ID NO: 1755 | CTGCCAAGGCAGGGTGGCC | SEQ ID NO: 4121 | CAGGCCACCTAAGGGTATAG | SEQ ID NO: 6487 |
| rs2508024 | TTCCTCTAAGGCCCAGTCACCAA | SEQ ID NO: 1756 | TTCCTCTAAGGCCCAGTCACCAG | SEQ ID NO: 4122 | GGTTTGGAAATTTTCACTACATA | SEQ ID NO: 6468 |
| rs3130275 | GGAAAGCTATAGGCTAATCTCACTGACAAA | SEQ ID NO: 1757 | GGAAAGCTATAGGCTAATCTCACTGACAAG | SEQ ID NO: 4123 | ATACCTGGAAAAATCCTAAATAAA | SEQ ID NO: 6489 |
| rs3131115 | GCTTTGGTTGCCACCACTAACTCA | SEQ ID NO: 1758 | GCTTTGGTTGCCACCACTAACTCG | SEQ ID NO: 4124 | CATGTGTAGACAGATGTCTTGAA | SEQ ID NO: 6490 |
| rs1131541 | CAGCCCTATAGCCACCCCAAGA | SEQ ID NO: 1759 | CAGCCCTATAGCCACCCCAAGT | SEQ ID NO: 4125 | TGCTCCATACTCTAACATCTAGC | SEQ ID NO: 6491 |
| rs7452206 | AACCCAAGGGCTATTCCTGCAT | SEQ ID NO: 1760 | AACCCAAGGGCTATTCCTGCAC | SEQ ID NO: 4126 | GAGCTAAGTAAGTAACAAAGGCC | SEQ ID NO: 6492 |
| rs2074470 | TTTGGACTTCGGACACCTCCAT | SEQ ID NO: 1761 | TTTGGACTTCGGACACCTCCAC | SEQ ID NO: 4127 | GTTGGAGAGGAGTAACTTTGTCT | SEQ ID NO: 6493 |
| rs7751953 | ACCCTCAAACCCCAGCTGACTT | SEQ ID NO: 1762 | ACCCTCAAACCCCAGCTGACTG | SEQ ID NO: 4128 | ATGCTTCATGTCTCCAAAATATA | SEQ ID NO: 6494 |
| rs2073148 | TAAGAGGCAAGGATGAGGCTGAGA | SEQ ID NO: 1763 | TAAGAGGCAAGGATGAGGCTGAGG | SEQ ID NO: 4129 | GCACAACCACGAAGATGAC | SEQ ID NO: 6495 |
| rs885910 | GAGGGACTAGGGTGTCCCCAAA | SEQ ID NO: 1764 | GAGGGACTAGGGTGTCCCCAAT | SEQ ID NO: 4130 | AACAGGTTAGATTCTCTCTTTGG | SEQ ID NO: 6496 |
| rs3094179 | CCCCAGGACCCCTGGCAA | SEQ ID NO: 1765 | CCCCAGGACCCCTGGCAG | SEQ ID NO: 4131 | GCTGTCTCATTACTGAGTGTGC | SEQ ID NO: 6497 |
| rs2301277 | AGCGGAGTGTAAGGCCAAGTGAA | SEQ ID NO: 1766 | AGCGGAGTGTAAGGCCAAGTGAG | SEQ ID NO: 4132 | GTAATTTATGTGCTCCTGAAAATT | SEQ ID NO: 6498 |
| rs2239885 | CAGAGGAGGAGCCCAACAGAATA | SEQ ID NO: 1767 | CAGAGGAGGAGCCCAACAGAATC | SEQ ID NO: 4133 | ACCAAACTTAACCAAGAATCAAC | SEQ ID NO: 6499 |
| rs356963 | TTTCACCATGGGTCAGCCCAC | SEQ ID NO: 1768 | TTTCACCATGGGTCAGCCCAG | SEQ ID NO: 4134 | CCTGGTAGGATGAGTGCAT | SEQ ID NO: 6500 |
| rs3132721 | TGGAAAGCAAGCACATATGCA | SEQ ID NO: 1769 | TGGAAAGCAAGCACATATGCG | SEQ ID NO: 4135 | TGAGTGGTCCACTGATATTACAT | SEQ ID NO: 6501 |
| rs928976 | CCAGGGGGAAGAGCAAAGGACT | SEQ ID NO: 1770 | CCAGGGGGAAGAGCAAAGGACC | SEQ ID NO: 4136 | GATCCTGGGAACTGGACAGT | SEQ ID NO: 6502 |
| rs3131784 | CTGTGGGTGCATTTGATGTGACT | SEQ ID NO: 1771 | CTGTGGGTGCATTTGATGTGACC | SEQ ID NO: 4137 | CTTGAATCTCTTCTGTTTTTTCC | SEQ ID NO: 6503 |
| rs3097652 | CACTTCAGTTGCAAAAGTCCTATTTCTTA | SEQ ID NO: 1772 | CACTTCAGTTGGAAAAGTCCTATTTCTTG | SEQ ID NO: 4138 | CTCATTTATTATGTTTCTTATTCCA | SEQ ID NO: 6504 |
| rs805304 | CATTCCGCCCTGCTAAGCCT | SEQ ID NO: 1773 | CATTCCGCCCTGCTAAGCCG | SEQ ID NO: 4139 | GCCCATTACATCCAGACTG | SEQ ID NO: 6505 |
| rs5017401 | TTCTGGGTCCACACGATGTTTTA | SEQ ID NO: 1774 | TTCTGGGTCCACACGATGTTTTC | SEQ ID NO: 4140 | TTTATATTTTCTTGTGTGTGGAAA | SEQ ID NO: 6506 |
| rs130067 | GGAGCGACAGAAGCTTCTGGAA | SEQ ID NO: 1775 | GGAGCGAOAGAAGCTTCTGGAC | SEQ ID NO: 4141 | GAATGTATCTGTGTGCAGACTTAG | SEQ ID NO: 6507 |
| rs2530716 | GGGGAGGGAGGACCCAAAGT | SEQ ID NO: 1776 | GGGGAGGGAGGACCCAAAGC | SEQ ID NO: 4142 | CTGGCCTCCCCTACTCAGTAT | SEQ ID NO: 6508 |
| rs635348 | TCAGCCTTTCCATCTTTGCTGTCC | SEQ ID NO: 1777 | TCAGCCTTTCCATCTTTGCTGTCG | SEQ ID NO: 4143 | AACAGTGCTTGTGGAATAAAAAT | SEQ ID NO: 6509 |
| rs464865 | TAAAAAAGAAAGGCAGGTCCGGA | SEQ ID NO: 1778 | TAAAAAAGAAAGGCAGGTCCGGG | SEQ ID NO: 4144 | GGTTTCTTTTTCTGGATTAGTTT | SEQ ID NO: 6510 |
| rs2844688 | TCATAAAGGCATGAGGTGGGACA | SEQ ID NO: 1779 | TCATAAAGGCATGAGGTGGGACG | SEQ ID NO: 4145 | AAGGAGCTTATGATTTTGTCTGT | SEQ ID NO: 6511 |
| rs1632938 | GCCTCCACTCAGGTCAGAACCA | SEQ ID NO: 1780 | GCCTCCACTCAGGTCAGAACCG | SEQ ID NO: 4146 | AGAGACTAGAACTTTCCAAGGAA | SEQ ID NO: 6512 |
| rs565876 | ACCATAGTAGGAAACCTGGATAGAGACA | SEQ ID NO: 1781 | ACCATAGTAGGAAACCTGGATAGAGACG | SEQ ID NO: 4147 | GGAAATTAGAATCAGGAATGTAGT | SEQ ID NO: 6513 |
| rs6911246 | CTATGGACTGAATTGTGTCCCCCT | SEQ ID NO: 1782 | CTATGGACTGAATTGTGTCCCCCC | SEQ ID NO: 4148 | CAAAATTCATATGTTGAAAATCTAA | SEQ ID NO: 6514 |
| rs3093557 | AGGGAGGGGGTGGGACAGA | SEQ ID NO: 1783 | AGGGAGGGGGTGGGACAGG | SEQ ID NO: 4149 | AGACCATCATCCCTAGGAGC | SEQ ID NO: 6515 |
| rs2516672 | CTTTGGGGTAGCTCCCTCTCAGA | SEQ ID NO: 1784 | CTTTGGGGTAGCTCCCTCTCAGG | SEQ ID NO: 4150 | TGTCCTGCAGGTCCTTACA | SEQ ID NO: 6516 |
| rs3095307 | GAGGCCAGCTGGGGCAAG | SEQ ID NO: 1785 | GAGGCCAGCTGGGGCAAC | SEQ ID NO: 4151 | TGAGGGACTCCATAAAGAGG | SEQ ID NO: 6517 |
| rs3814959 | CGGTTCCTCTCCAAACCTGGTT | SEQ ID NO: 1786 | CGGTTCCTCTCCAAACCTGGTC | SEQ ID NO: 4152 | CCCTTCTCACTGACTAGTGATC | SEQ ID NO: 6518 |
| rs1611493 | GCTGTTAGTCACTAAATTTGGGGGTAGTT | SEQ ID NO: 1787 | GCTGTAAGTCACTAAATTTGGGGGTAGTG | SEQ ID NO: 4153 | TTTACACAGCAACAGATAACTAATG | SEQ ID NO: 6519 |
| rs1233492 | GAATATTCTCCAATCTTGAAAATGGCT | SEQ ID NO: 1788 | GAATATTCTCCAATCTTGAAAATGGCC | SEQ ID NO: 4154 | ATAAGTCTTTAACTCTCTAGAGTTGC | SEQ ID NO: 6520 |
| rs6456890 | GGGGCATCAATAATTAATGCTTATTTATT | SEQ ID NO: 1789 | GGGGCATCAATAATTAATGCTTATTTATC | SEQ ID NO: 4155 | TAACAGACTCGTTGATGTGTATAA | SEQ ID NO: 6521 |
| rs2844680 | AATTCTCAGGGGAAATTTTTTACCCA | SEQ ID NO: 1790 | AATTCTCAGGGGAAATTTTTTACCCC | SEQ ID NO: 4156 | TCCATTATTATAAACTGTGATAATCTT | SEQ ID NO: 6522 |
| rs769177 | GTTACTCATCCCAGAAGCACCCAT | SEQ ID NO: 1791 | GTTACTCATCCCAGAAGCACCCAC | SEQ ID NO: 4157 | GGACCTAAAGTGTGAAAAAAAG | SEQ ID NO: 6523 |
| rs2844659 | GTAGAGGCAGAGGGGGCTGGA | SEQ ID NO: 1792 | GTAGAGGCAGAGGGGGCTGGG | SEQ ID NO: 4158 | TCTTACTCAAAACAGTGAGCTTC | SEQ ID NO: 6524 |
| rs2516698 | AACAGAATCCAAAGGCCGGCA | SEQ ID NO: 1793 | AACAGAATCCAAAGGCCGGCG | SEQ ID NO: 4159 | AACTGTAGGAAATGAGTGCAGTA | SEQ ID NO: 6525 |
| rs204890 | GGCCCCCTCTCGTTTGAGGA | SEQ ID NO: 1794 | GGCCCCCTCTCGTTTGAGGG | SEQ ID NO: 4160 | ATGAGTTGGTCTTCTCCCC | SEQ ID NO: 6526 |
| rs6457699 | TGATTGCCTGTCCAGCTGTTGAA | SEQ ID NO: 1795 | TGATTGCCTGTCCAGCTGTTGAG | SEQ ID NO: 4161 | GGTGGGTGAGAAAATCCAAG | SEQ ID NO: 6527 |
| rs2071787 | ACTGCGCTAAGGAGGCAACTGTC | SEQ ID NO: 1796 | ACTGCGCTAAGGAGGCAACTGTG | SEQ ID NO: 4162 | ACGGCTCTACCAAGATGATTAT | SEQ ID NO: 6528 |
| rs892666 | GGAAAATGGGACCAACGCTAAAAT | SEQ ID NO: 1797 | GGAAAATGGGACCAACGCTAAAAC | SEQ ID NO: 4163 | TCCCTCTCCCTCTTGTGAG | SEQ ID NO: 6529 |
| rs2517894 | GCACTCCTTTCCCACCGTTTTT | SEQ ID NO: 1798 | GCACTCCTTTCCCACCGTTTTC | SEQ ID NO: 4164 | CACCTCTCAATTCTGTTCTCAG | SEQ ID NO: 6530 |
| rs1264348 | CAGTGACAAAACAGGAAGAACAAAAAAA | SEQ ID NO: 1799 | CAGTGACAAAACAGGAAGAACAAAAAAC | SEQ ID NO: 4165 | ACATGTTAATATCTCAAGAAAGTATCC | SEQ ID NO: 6531 |
| rs2394734 | TCTTCATGCTATTTATTACTGTCTGCAA | SEQ ID NO: 1800 | TCTTCATGCTATTTATTACTGTCTGCAG | SEQ ID NO: 4166 | GATCTATTTATGGACTTTTGATGTG | SEQ ID NO: 6532 |
| rs3749950 | GTGAGTGGTGGGCACAGGACAC | SEQ ID NO: 1801 | GTGAGTGGTGGGCACAGGACAG | SEQ ID NO: 4167 | GGCTGATGTTGATGGAGTG | SEQ ID NO: 6533 |
| rs1264350 | CAGGGAAAGCTGAGTCATGATTCTA | SEQ ID NO: 1802 | CAGGGAAAGCTGAGTCATGATTCTG | SEQ ID NO: 4168 | AAAAATTAAGTCAGATGTGCTCA | SEQ ID NO: 6534 |
| rs2517407 | AACCTTTGGAGGAATGGTCAGACTA | SEQ ID NO: 1803 | AACCAAGGAGGAATGGTCAGACTG | SEQ ID NO: 4169 | TTTCCAAAGTGCTGTACCATTT | SEQ ID NO: 6535 |
| rs3117426 | GCAGCTACTGGAATGAATTAGAGTTTAT | SEQ ID NO: 1804 | GCAGCTACTGGAATGAATTAGAGTTTAC | SEQ ID NO: 4170 | TACATAACATATTTGCACATGTACC | SEQ ID NO: 6536 |
| rs3134767 | TTCTGCCAAAATGAAGCCAACATA | SEQ ID NO: 1805 | TTCTGCCAAAATGAAGCCAACATG | SEQ ID NO: 4171 | GATAAGGATATCAAAATGATTACCA | SEQ ID NO: 6537 |
| rs2535238 | GTGACCAGGGCCCTGAACTCT | SEQ ID NO: 1806 | GTGACCAGGGCCCTGAACTCG | SEQ ID NO: 4172 | AAGCAAAATTGTTCTCAATTGT | SEQ ID NO: 6538 |
| rs805282 | AGCACAGTCCCCAGGCCTCAA | SEQ ID NO: 1807 | AGCACAGTCCCCAGGCCTCAC | SEQ ID NO: 4173 | AGGGTTGGGGTTTGTTAAC | SEQ ID NO: 6539 |
| rs401618 | TCCTTCTTGCTGAGTAATGTTCTAGTTTTA | SEQ ID NO: 1808 | TCCTTCTTGCTGAGTAATGTTCTAGTTTTG | SEQ ID NO: 4174 | CCCAGCGTTAATATTTTAAGTCT | SEQ ID NO: 6540 |
| rs550605 | TGTTCTGCTCCTGCAGAGGTCAT | SEQ ID NO: 1809 | TGTTCTGCTCCTGCAGAGGTCAC | SEQ ID NO: 4175 | AGATCTTCAGCCAGGGATC | SEQ ID NO: 6541 |
| rs2021722 | GGCTTACTTTCCCCACGGAGCT | SEQ ID NO: 1810 | GGCTTACTTTCCCCACGGAGCC | SEQ ID NO: 4176 | AGCATCAGTGGTGCTCTGA | SEQ ID NO: 6542 |
| rs885950 | CAGAAAGACGCAGAGGAGGTGGA | SEQ ID NO: 1811 | CAGAAAGACGCAGAGGAGGTGGC | SEQ ID NO: 4177 | AGTGATTTGTGGAGGGGTG | SEQ ID NO: 6543 |
| rs2516489 | CCAAGGGCTCACCATTGACAGT | SEQ ID NO: 1812 | CCAAGGGCTCACCATTGACAGC | SEQ ID NO: 4178 | GTTGACCATGGCTGATAGTATG | SEQ ID NO: 6544 |
| rs2239842 | GAAAATGGGGATGGGGTGAAA | SEQ ID NO: 1813 | GAAAATGGGGATGGGGTGAAG | SEQ ID NO: 4179 | ACAGAGAGAGAATATCCCCTCT | SEQ ID NO: 6545 |
| rs3117235 | CCTTCATGGCCATAGCCACAA | SEQ ID NO: 1814 | CCTTCATGGCCATAGCCACAC | SEQ ID NO: 4180 | GTGAGAGGTTCAAGTGACTGAC | SEQ ID NO: 6546 |
| rs446145 | GCCTACCGAGTGTTGGCCAC | SEQ ID NO: 1815 | GCCTACCGAGTGTTGGCCAG | SEQ ID NO: 4181 | TTGCTCTTATTCTTAGGGAGTTT | SEQ ID NO: 6547 |
| rs2894260 | AGCTCAAGGCTCAGGGCCTTTA | SEQ ID NO: 1816 | AGCTCAAGGCTCAGGGCCTTTG | SEQ ID NO: 4182 | TGACCCCTTCTTCCAAATATAC | SEQ ID NO: 6548 |
| rs2143468 | CACAATATTCAGTATTTTGGCAGGATAA | SEQ ID NO: 1817 | CACAATATTCAGTATTTTGGCAGGATAT | SEQ ID NO: 4183 | TACATCGTAAGTTTTTTTATCTTC | SEQ ID NO: 6549 |
| rs3873260 | TTGAGTGCTATTTCTTTGTGGCATCA | SEQ ID NO: 1818 | TTGAGTGCTATTTCTTTGTGGCATCG | SEQ ID NO: 4184 | GACAAATTTACAATAATACATTTCCT | SEQ ID NO: 6550 |
| rs1050391 | CAGGCAAGGACTGCAGCTAGACA | SEQ ID NO: 1819 | CAGGCAAGGACTGCAGCTAGACG | SEQ ID NO: 4185 | AGAAGCAGAGCCAGATCCAG | SEQ ID NO: 6551 |
| rs3094001 | GGGCAGGATCTGGATGGGACA | SEQ ID NO: 1820 | GGGCAGGATCTGGATGGGACG | SEQ ID NO: 4186 | CTCTGACAACACCTCACCTAGT | SEQ ID NO: 6552 |
| rs715044 | GCTTACAGCTCAGGAATCCACCAAT | SEQ ID NO: 1821 | GCTTACAGCTCAGGAATCCACCAAG | SEQ ID NO: 4187 | TACAGATAAATACTTGGGATTCATC | SEQ ID NO: 6553 |
| rs1810640 | ACCAGGTGTGCCCTCATATGCTT | SEQ ID NO: 1822 | ACCAGGTGTGCCCTCATATGCTC | SEQ ID NO: 4188 | ATTCCAGAAGCTTCACAAGAC | SEQ ID NO: 6554 |
| rs887468 | TGACATTGCACTGTAATAATAAAGGTGTA | SEQ ID NO: 1823 | TGACATTGCACTGTAATAATAAAGGTGTG | SEQ ID NO: 4189 | GTGAAGTATCTTTTTATGGTGACTT | SEQ ID NO: 6555 |
| rs755714 | AACCTCATCCCACCTTGGCAAT | SEQ ID NO: 1824 | AACCTCATCCCACCTTGGCAAC | SEQ ID NO: 4190 | CTAAACCAAGGCCATCTACATT | SEQ ID NO: 6556 |
| rs1265777 | CCTTCAGGACTCACATCTTAGTCACAG | SEQ ID NO: 1825 | CCTTCAGGACTCACATCTTAGTCACAC | SEQ ID NO: 4191 | TAGAAGAAGTTAATGACTTATGTCTT | SEQ ID NO: 6557 |
| rs999265 | AGGATAGCCCTGCTAGGCAAGGA | SEQ ID NO: 1826 | AGGATAGCCCTGCTAGGCAAGGG | SEQ ID NO: 4192 | CAGTTAAAAAAAGTATAAATAAATTGA | SEQ ID NO: 6558 |
| rs2021729 | AGCCTCCCTTTCCCAGGCTTATT | SEQ ID NO: 1827 | AGCCTCCCTTTCCCAGGCTTATC | SEQ ID NO: 4193 | TCATCCAGCAGCTCCTTCT | SEQ ID NO: 6559 |
| rs7452076 | AAATAGCTCAGAGAATGCCAATGGT | SEQ ID NO: 1828 | AAATAGCTCAGAGAATGCCAATGGC | SEQ ID NO: 4194 | GAGGAAGGAAGGATGAGTAGAT | SEQ ID NO: 6560 |
| rs743399 | CCTCCCCCCACCAAGGAT | SEQ ID NO: 1829 | CCTCCCCCCACCAAGGAC | SEQ ID NO: 4195 | CAGTAAACCCACCACTCCAG | SEQ ID NO: 6561 |
| rs2256594 | GGGCGACTTTTCCCCCTTTAAAA | SEQ ID NO: 1830 | GGGCGACTTTTCCCCCTTAAAG | SEQ ID NO: 4196 | GTCTTGCAGCAATTTTTTTCT | SEQ ID NO: 6562 |
| rs1046080 | CATCAGCCCCAACACCAGAGAA | SEQ ID NO: 1831 | CATCAGCCCCAACACCAGAGAC | SEQ ID NO: 4197 | GAACCTGAAGAGCCAGCAC | SEQ ID NO: 6563 |
| rs2523807 | GTTTTTTTCAAAAACTGGCTATTTGAT | SEQ ID NO: 1832 | GTTTTTTTCAAAAACTGGCTATTTGAC | SEQ ID NO: 4198 | GGCATTCTATAATAAACTTAACATCT | SEQ ID NO: 6564 |
| rs3094647 | GCAGTGTCGACCTCCTGGGT | SEQ ID NO: 1833 | GCAGTGTCGACCTCCTGGGG | SEQ ID NO: 4199 | CAAGGGACCCACTGATCTC | SEQ ID NO: 6565 |
| rs421446 | GACGCACGAACCCCTCCCTT | SEQ ID NO: 1834 | GACGCACGAACCCCTCCCTC | SEQ ID NO: 4200 | AAAAAGACCCGGAGTGTCAC | SEQ ID NO: 6566 |
| rs6415110 | GCAACTGCTATACTACAGTAGTCAACCTGA | SEQ ID NO: 1835 | GCAACTGCTATACTACAGTAGTCAACCTGG | SEQ ID NO: 4201 | AACTGAGATGGCTAGCTACTAAG | SEQ ID NO: 6567 |
| rs2285805 | ACCCAGGGTCCTCGCCCCG | SEQ ID NO: 1836 | ACCCAGGGTCCTCGCCCCC | SEQ ID NO: 4202 | CATCCTTTGCTTTTCTCTCC | SEQ ID NO: 6568 |
| rs2242955 | CATAGCACAGGGAGGGTTTCCCT | SEQ ID NO: 1837 | CATAGCACAGGGAGGGTTTCCCC | SEQ ID NO: 4203 | GAGGCAACTCTAGCAGAATTG | SEQ ID NO: 6569 |
| rs29233 | GGCAGAATCACTCTCCAAAAAGACTTACG | SEQ ID NO: 1838 | GGCAGAATCACTCTCCAAAAAGACTTACC | SEQ ID NO: 4204 | GTGTTTATCTGAATATTTTCTTTTAGC | SEQ ID NO: 6570 |
| rs4711312 | AAGACGAGGCAAAGACACCTAGAAT | SEQ ID NO: 1839 | AAGACGAGGCAAAGACACCTAGAAC | SEQ ID NO: 4205 | CAAACTACAGACTGAATTTCTTTC | SEQ ID NO: 6571 |
| rs1264327 | CTGCCAGGCAAGCTACAGCCT | SEQ ID NO: 1840 | CTGCCAGGCAAGCTACAGCCC | SEQ ID NO: 4206 | TCATACCCTGGTGGGACAT | SEQ ID NO: 6572 |
| rs2261033 | TGCAACGTAGAAAGGCTTGATTATAAAT | SEQ ID NO: 1841 | TGCAAGGTAGAAAGGCTTGATTATAAAC | SEQ ID NO: 4207 | CATACCAGGCAGAAAAAAATTAT | SEQ ID NO: 6573 |
| rs805288 | CAAAGGGCTAGGCTCACACCCT | SEQ ID NO: 1842 | CAAAGGGCTAGGCTCACACCCC | SEQ ID NO: 4208 | CCTCTGTACCCCACCTCCT | SEQ ID NO: 6574 |
| rs3106189 | CGCGGTAAGGCTGGGGTA | SEQ ID NO: 1843 | CGCGGTAAGGCTGGGGTG | SEQ ID NO: 4209 | CCTGGTGAGAACTGGAGAG | SEQ ID NO: 6575 |
| rs2157338 | CCCAATATGCAATTAACAAAGAAGATAACA | SEQ ID NO: 1844 | CCCAATATGCAATTAACAAAGAAGATAACG | SEQ ID NO: 4210 | TAGAGAGTAATAGTGCACTGAAGAA | SEQ ID NO: 6576 |
| rs805299 | TCCCTGCCCCAATACCTAAAAAGTA | SEQ ID NO: 1845 | TCCCTGCCCCAATACCTAAAAAGTT | SEQ ID NO: 4211 | ACACACACATTCACACCTGTC | SEQ ID NO: 6577 |
| rs2074505 | CCCCGCACCCCACAGGAT | SEQ ID NO: 1846 | CCCCGCACCCCACAGGAC | SEQ ID NO: 4212 | CTAGTAGTGGTGAGTGGGCAAT | SEQ ID NO: 6578 |
| rs1042338 | CTGCCTCGGGAGTTCTGACCA | SEQ ID NO: 1847 | CTGCCTCGGGAGTTCTGACCG | SEQ ID NO: 4213 | GTGTTAAGCCTCTGTCTCTGG | SEQ ID NO: 6579 |
| rs1059510 | CGCACAGATTTTCCGAGTGAAT | SEQ ID NO: 1848 | CGCACAGATTTTCCGAGTGAAC | SEQ ID NO: 4214 | CTACTACAATCAGAGCGAGGC | SEQ ID NO: 6580 |
| rs4678 | GGAGGCTCATCCATCAGCTGCT | SEQ ID NO: 1849 | GGAGGCTCATCCATCAGCTGCC | SEQ ID NO: 4215 | CTTGTCCAGTTTTGACAATTC | SEQ ID NO: 6581 |
| rs396090 | CCTCCCATCCTCCAAAACTTCCTT | SEQ ID NO: 1850 | CCTCCCATCCTCCAAAACTTCCTC | SEQ ID NO: 4216 | AGTGAATCCTTCCACCACTC | SEQ ID NO: 6582 |
| rs1233419 | TTGTTGGAAAATTGCAAGTTGTATGA | SEQ ID NO: 1851 | TTGTTGGAAAATTGCAAGTTGTATGG | SEQ ID NO: 4217 | AATGCCAACCTAGAATTCTTTAC | SEQ ID NO: 6583 |
| rs1265063 | CACCTTCCTCAGAGCCCACCTTT | SEQ ID NO: 1852 | CACCTTCCTCAGAGCCCACCTTC | SEQ ID NO: 4218 | CCATCACCAAGCACACTC | SEQ ID NO: 6584 |
| rs3117223 | AGTGCTGAACATCACGTGGCTGT | SEQ ID NO: 1853 | AGTGCTGAACATCACGTGGCTGC | SEQ ID NO: 4219 | AGTCATTGAGGGTATTGCAGA | SEQ ID NO: 6585 |
| rs2269475 | GCCCCCGCAAATCACCCA | SEQ ID NO: 1854 | GCCCCCGCAAATCACCCG | SEQ ID NO: 4220 | TTCTCACCAATATCGCCATT | SEQ ID NO: 6586 |
| rs2235499 | AGTCCCTGCCTGTGTCCCCCA | SEQ ID NO: 1855 | AGTCCCTGCCTGTGTCCCCCC | SEQ ID NO: 4221 | CTCTTGACTGTCCCCCACTT | SEQ ID NO: 6587 |
| rs6457020 | TACTTGGATATAACACCAAAGATACAAA | SEQ ID NO: 1856 | TTTCTTGGATATAACACCAAAGATACAAC | SEQ ID NO: 4222 | ACAAAGAAAAAATTGATAAATGG | SEQ ID NO: 6588 |
| rs185819 | GTCACCCACAGGTTTCCCAGGT | SEQ ID NO: 1857 | GTCACCCACAGGTTTCCCAGGC | SEQ ID NO: 4223 | GTCACTCTGAGGCACTAGGAA | SEQ ID NO: 6589 |
| rs2273611 | GGGGGCCACAGGAAAGGCT | SEQ ID NO: 1858 | GGGGGCCACAGGAAAGGCC | SEQ ID NO: 4224 | GATGGATGGAGGTAGGGAG | SEQ ID NO: 6590 |
| rs1632879 | GGCTGGGTTGCTGGATGATTCT | SEQ ID NO: 1859 | GGCTGGGTTGCTGGATGATTCC | SEQ ID NO: 4225 | CTCACACAACGGACTAGG | SEQ ID NO: 6591 |
| rs3093551 | CCTCCCTGGGCCTCAGTTTCT | SEQ ID NO: 1860 | CCTCCCTGGGCCTCAGTTTCC | SEQ ID NO: 4226 | TCTCTGCTGAGCCAGAAGAT | SEQ ID NO: 6592 |
| rs130072 | GCCTTGGCATGCTCCCTCT | SEQ ID NO: 1881 | GCCTTGGCATGCTCCCTCC | SEQ ID NO: 4227 | CTCCTCTCTGTGTCTGAGAGTT | SEQ ID NO: 6593 |
| rs2295667 | CCTCCCAGCCACAGCTCGT | SEQ ID NO: 1862 | CCTCCCAGCCACAGCTCGG | SEQ ID NO: 4228 | TATCCCAAGCTGGGTAATGT | SEQ ID NO: 6594 |
| rs2517466 | CCCTGCCGCTTTCTGTGACT | SEQ ID NO: 1863 | CGCTGCCGCTTTCTGTGACC | SEQ ID NO: 4229 | CAGTGTTCCCCAAACCTTGT | SEQ ID NO: 6595 |
| rs28T724 | TTCAAAATAGTTGACAGCTGGTATGGT | SEQ ID NO: 1864 | TTCAAAATAGTTGACAGCTGGTATGGC | SEQ ID NO: 4230 | AAATAAAATTTTCCCAATTATGC | SEQ ID NO: 6596 |
| rs7769073 | GTGGGCACTGGGGGGAAGT | SEQ ID NO: 1865 | GTGGGCACTGGGGGGAAGA | SEQ ID NO: 4231 | GGGAGGGTGATATCAGCAC | SEQ ID NO: 6597 |
| rs3130780 | CCATAAAGAAGCTCTGTCAATTTTTAGCTT | SEQ ID NO: 1866 | CCATAAAGAAGCTCTGTCAATTTTTAGCTG | SEQ ID NO: 4232 | TCTTTGAAGATTTTTAAAACATTAATT | SEQ ID NO: 6598 |
| rs4713354 | GAGGAAAGGCGCTCTGGCCT | SEQ ID NO: 1867 | GAGGAAAGGCGCTCTGGCCG | SEQ ID NO: 4233 | GTCTATGACACAGCCCCTCTAT | SEQ ID NO: 6599 |
| rs2021720 | CTCCTGGTGGGCGACCGTG | SEQ ID NO: 1868 | CTCCTGGTGGGCGACCGTC | SEQ ID NO: 4234 | GACCAGAAATTAGATTTGAGTC | SEQ ID NO: 6600 |
| rs6456879 | TCAGTGAGCACATTTACTTCCAAGAGTT | SEQ ID NO: 1869 | TCAGTGAGCACATTTACTTCCAAGAGTA | SEQ ID NO: 4235 | ATCAATCACTTCCTAATATTAATAAA | SEQ ID NO: 6601 |
| rs6913528 | CAAAGCCGGTTTTGCTCACTGTT | SEQ ID NO: 1870 | CAAAGCCGGTTTTGCTCACTGTC | SEQ ID NO: 4236 | CTTGTTAGCACCCACTGAAT | SEQ ID NO: 6602 |
| rs6929434 | CCTCTTGGTAAAGTCAGCTGTAGGAGT | SEQ ID NO: 1871 | CCTCTTGGTAAAGTCAGCTGTAGGAGA | SEQ ID NO: 4237 | ATAGTAGCCAACATTCATCTAGTG | SEQ ID NO: 6603 |
| rs207T99 | GCGGGGTCTGGGTTTTCTTGTT | SEQ ID NO: 1872 | GCGGGGTCTGGGTTTTCTTGTC | SEQ ID NO: 4238 | CACTGGGAGTCAAGCC | SEQ ID NO: 6604 |
| rs518374 | AAATAGAAAAACAATCAATTGGGAAAAA | SEQ ID NO: 1873 | AAATAGAAAAACAATCAATTGGGAAAAC | SEQ ID NO: 4239 | GTGTTTGAAAGAGTATATCAATAAGA | SEQ ID NO: 6605 |
| rs446735 | GGTCATCCCAAGGGCTTTCCAA | SEQ ID NO: 1874 | GGTCATCCCAAGGGCTTCCAC | SEQ ID NO: 4240 | CAGTTCCTGAGCTCCATG | SEQ ID NO: 6606 |
| rs2075799 | CTGTCCCTGGGGCTGGAGACA | SEQ ID NO: 1575 | CTGTCCCTGGGGCTGGAGACG | SEQ ID NO: 4241 | GTGATGACTGCCCTGATAAAG | SEQ ID NO: 6607 |
| rs7757012 | GGGAGGCCTACCTGGGGCT | SEQ ID NO: 1676 | GGGAGGCCTACCTGGGGCC | SEQ ID NO: 4242 | CTGCAGTCCAAGTACGCTG | SEQ ID NO: 6608 |
| rs3093984 | CTGGATGTGCTCGAAGGTCCAT | SEQ ID NO: 1877 | CTGGATGTGCTCGAAGGTCCAC | SEQ ID NO: 4243 | GAGGGTTCATGTCTAGGTCC | SEQ ID NO: 6609 |
| rs805258 | GGAAAGAGATCCAGCCTGGCTT | SEQ ID NO: 1878 | GGAAAGAGATCCAGCCTGGCTC | SEQ ID NO: 4244 | CCTAAGTCAGCCTCTTCACC | SEQ ID NO: 6610 |
| rs2071351 | GCCACTCCAGGTAAGAGCCGAA | SEQ ID NO: 1879 | GCCACTCCAGGTAAGAGCCGAG | SEQ ID NO: 4245 | CTCAGGGAACAATTCCTAGG | SEQ ID NO: 6611 |
| rs6938130 | TGATAGATTAAATGTTTCTAGGAATTTGCT | SEQ ID NO: 1880 | TGATAGATTAAATGTTTCTAGGAATTTGCA | SEQ ID NO: 4246 | ATCTCACCTAGGTTTTCAAATTT | SEQ ID NO: 6612 |
| rs3130388 | TGGAAATATTCGATTGCTTAGAGTGACA | SEQ ID NO: 1881 | TGGAAATATTCGATTGCTTAGAGTGACC | SEQ ID NO: 4247 | ATGATTACATAACATCCTTTATTATC | SEQ ID NO: 6613 |
| rs1061815 | GGACATGGGGGTGGGGTGG | SEQ ID NO: 1882 | GGACATGGGGGTGGGGTGC | SEQ ID NO: 4248 | CTCCCCACCTCCTCACATTAT | SEQ ID NO: 6614 |
| rs241455 | TGAAAAACACTTAGCATAGCTCCTACTCA | SEQ ID NO: 1883 | TGAAAAACACTTAGCATAGCTCCTACTCC | SEQ ID NO: 4249 | ATTAAAACTCTATAAATGGTAGCTGT | SEQ ID NO: 6615 |
| rs3891288 | TCCATATTGTAATAATTGGCAGGATCA | SEQ ID NO: 1884 | TCCATATTGTAATAATTGGCAGGATCT | SEQ ID NO: 4250 | CTTCTTTTAAAGGTTAAATAATATTCA | SEQ ID NO: 6616 |
| rs1655938 | ACCTGGAGAACGGGAAGGAGAT | SEQ ID NO: 1885 | ACCTGGAGAACGGGAAGGAGAC | SEQ ID NO: 4251 | GGTACCAGGGAACACAAGAC | SEQ ID NO: 6617 |
| rs3131102 | CGCTTCCAGCGAGTGACAAGA | SEQ ID NO: 1886 | CGCTTCCAGCGAGTGACAAGG | SEQ ID NO: 4252 | AGCTGTTTCCTCTTGGAAAG | SEQ ID NO: 6616 |
| rs6457566 | CCAAGCGAAAAATTATGTATCATCTATACA | SEQ ID NO: 1887 | CCAAGCGAAAAATTATGTATCATCTATACG | SEQ ID NO: 4253 | ACATGTCAATTTTATTGTTAGAGC | SEQ ID NO: 6619 |
| rs204887 | CGCCCCTCGTGGAGGCCA | SEQ ID NO: 1888 | CGCCCCTCGTGGAGGCCG | SEQ ID NO: 4254 | ACAGGTGCATCTTGTATTTGC | SEQ ID NO: 6620 |
| rs2844725 | TCTTTCTTTTCGAAGCGCAGTACA | SEQ ID NO: 1889 | TCTTTCTTTTCGAAGCGCAGTTTCG | SEQ ID NO: 4255 | TCTTCTTGCTCAGGCTGG | SEQ ID NO: 6621 |
| rs2074491 | GGGTGGGTGACTGGGGACTTT | SEQ ID NO: 1890 | GGGTGGGTGACTGGGGACTTC | SEQ ID NO: 4256 | GCTGATTGGCTTCTCTAGAA | SEQ ID NO: 6622 |
| rs1233425 | CAGGCTGGAGCAGAGAGCCATC | SEQ ID NO: 1891 | CAGGCTGGAGCAGAGAGCCATG | SEQ ID NO: 4257 | CTCTCAGGTCATCACACACC | SEQ ID NO: 6623 |
| rs4569 | TTCTACTTTATTTCATATTCCCACCACA | SEQ ID NO: 1892 | TTCTACTTTATTTCATATTCCCACCACG | SEQ ID NO: 4258 | TAACGACTCCTTTAATTTAAACTAAA | SEQ ID NO: 6624 |
| rs2072109 | AAAGCCTCTCTGGCGGCATCG | SEQ ID NO: 1893 | AAAGCCTCTCTGGCGGCATCC | SEQ ID NO: 4259 | GGGGTATCAGAGGAAGGTAG | SEQ ID NO: 6625 |
| rs3130216 | GTGTGGGGCTATTGCACTTAGAATA | SEQ ID NO: 1894 | GTGTGGGGCTATTGCACTTAGAATG | SEQ ID NO: 4260 | GGAAAAATAATATGTAAACATTTTTG | SEQ ID NO: 6626 |
| rs3757340 | TCAGTAATAAGGTGAAATGGCCATAA | SEQ ID NO: 1895 | TCAGTAATAAGGTGAAATGGCCATAG | SEQ ID NO: 4261 | GAGAGAGAAAATCCAGAAAACAC | SEQ ID NO: 6627 |
| rs3116838 | AATTGAAGCTAAACATTTTTTCTTTAGGT | SEQ ID NO: 1896 | AATTGAAGCTAAACATTTTTTCTTTAGGC | SEQ ID NO: 4262 | AACATTTTGAAATGACTAAATTCA | SEQ ID NO: 6628 |
| rs7451419 | GGTCAATGGCCTGCCAGCA | SEQ ID NO: 1897 | GGTCAATGGCCTGCCAGCG | SEQ ID NO: 4263 | CTGTTGGCATGATCTTTAACC | SEQ ID NO: 6629 |
| rs1475865 | AGTTTGCCATCCACCGCCTA | SEQ ID NO: 1898 | AGTGCCATCCACCGCCTG | SEQ ID NO: 4264 | CCCAGGACATCATCATCTAC | SEQ ID NO: 6630 |
| rs1264315 | CACCTCAGGCAGACCAGGCA | SEQ ID NO: 1899 | CACCTCAGGCAGACCAGGCG | SEQ ID NO: 4265 | GTACATGTAAATCCCCTCCC | SEQ ID NO: 6631 |
| rs2269610 | GCCCTTCAAGTGGTGTGGGC | SEQ ID NO: 1900 | GCCCTTCAAGTGGTGTGGGG | SEQ ID NO: 4266 | ACCTTAAGTTGCCACCTTCT | SEQ ID NO: 6632 |
| rs3129012 | CTGGTGCAGGCCTGGGGCA | SEQ ID NO: 1901 | CTGGTGCAGGCCTGGGGGG | SEQ ID NO: 4267 | AGTACTTTAGCTTGTCTAGATT | SEQ ID NO: 6633 |
| rs3132935 | AACAGGATACAGGAGGAGGACTTAAGTA | SEQ ID NO: 1902 | AACAGGATACAGGAGGAGGACTTAAGTG | SEQ ID NO: 4268 | AATATTGAAATTCTTGGGTTAGG | SEQ ID NO: 6634 |
| rs2074473 | GACTGTGAGCCTGGGGTGCTA | SEQ ID NO: 1903 | GACTGTGAGCCTGGGGTGCTG | SEQ ID NO: 4269 | GCAAGGTCTACTGGGAAGTG | SEQ ID NO: 6635 |
| rs3130553 | GTGGGAAAGAGGAGTCGCT | SEQ ID NO: 1904 | GTGGGAAAGAGGAGTCGCC | SEQ ID NO: 4270 | CCACTGGAGAAGAGAGAGAAAG | SEQ ID NO: 6636 |
| rs2534672 | GCTTCACCAAGCCCCTTTCGC | SEQ ID NO: 1905 | GCTTCACCAAGCCCCTTTCGG | SEQ ID NO: 4271 | CTCTCACTGGCTTGTTCCTT | SEQ ID NO: 6637 |
| rs1573294 | GTTTTTGTGCCGAGCCCCCA | SEQ ID NO: 1906 | GTTTTTGTGCCGAGCCCCCG | SEQ ID NO: 4272 | GCTTCTTGATGGGCTCTG | SEQ ID NO: 6638 |
| rs7773534 | TGGGTATGAAAATGCAGTTAGATAGAAT | SEQ ID NO: 1907 | TGGGTATGAAAATGCAGTTAGATAGAAC | SEQ ID NO: 4273 | AAGAACTTCTAGTATTCAACAGTAAA | SEQ ID NO: 6639 |
| rs1737079 | TGTTCTACACTTAGAGTCAAAGCAAATTAT | SEQ ID NO: 1908 | TGTTCTACACTTAGAGTCAAAGCAAATTAC | SEQ ID NO: 4274 | TAACTGTAAGATGTTCAAAGATGAA | SEQ ID NO: 6640 |
| rs805297 | AGCAAAGACTTGCACATGGTAAT | SEQ ID NO: 1909 | AGCAAAGACTTGCACATGGTAAG | SEQ ID NO: 4275 | ATTAATAGTAATGTTTGCAAAGCA | SEQ ID NO: 6641 |
| rs805298 | CGGGGGTGGAGGCATTACCT | SEQ ID NO: 1910 | CGGGGGTGGAGGCATTACCG | SEQ ID NO: 4276 | GTGCCCTCTTCTCTTAACCTG | SEQ ID NO: 6642 |
| rs3749951 | AGTCCCCCACTCCCCCAGCTG | SEQ ID NO: 1911 | AGTCCCCCACTCCCCCAGCTC | SEQ ID NO: 4277 | CTTCTTGGCCTCTTCCAG | SEQ ID NO: 6643 |
| rs3094208 | GGAGGGGGCACTGGGGTAA | SEQ ID NO: 1912 | GGAGGGGGCACTGGGGTAG | SEQ ID NO: 4278 | GGAGGAGTGGACTAGAAGGTG | SEQ ID NO: 6644 |
| rs805263 | CTCCCCAGTTTCTGGGACACCT | SEQ ID NO: 1913 | CTCCCCAGTTTCTGGGACACCG | SEQ ID NO: 4279 | CTTGGTGAGTGAAACTTGGG | SEQ ID NO: 6645 |
| rs3135330 | AAAAGTACAAGCGGCCCCGTT | SEQ ID NO: 1914 | AAAAGTACAAGCGGCCCCGTC | SEQ ID NO: 4280 | CAATGACACAGCAACTTTGC | SEQ ID NO: 6646 |
| rs734181 | CACCGTCCCACCCACACCG | SEQ ID NO: 1915 | CACCGTCCCACCCACAGCC | SEQ ID NO: 4281 | GTGTCAGAAACTGGAGCCATA | SEQ ID NO: 6647 |
| rs1050673 | TTCCCCAAGTTAGACAGGAAGAGATA | SEQ ID NO: 1916 | TTCCCCAAGTTAGACAGGAAGAGATG | SEQ ID NO: 4282 | GAAGGCTAGTGCTTGGAGAG | SEQ ID NO: 6648 |
| rs3129948 | GAAAACTGGAAGGTTAGGTAAAACTGTGT | SEQ ID NO: 1917 | GAAAACTGGAAGGTTAGGTATAACTGTGG | SEQ ID NO: 4283 | ACACGATGTGTAAGTCCTAGAAG | SEQ ID NO: 6649 |
| rs2517436 | AAAAAATGATAAGTATGCAAGGCAATAGA | SEQ ID NO: 1918 | AAAAAATGATAAGTATGCAAGGCAATAGG | SEQ ID NO: 4264 | ATGTTAATTTGATTTAATAATTTCAA | SEQ ID NO: 6650 |
| rs2242664 | GTTGGTCCATGGAAAGCAGCGT | SEQ ID NO: 1919 | GTTGGTCCATGGAAAGCAGCGC | SEQ ID NO: 4285 | CCAGAGCTGGAAGGGAGAG | SEQ ID NO: 6651 |
| rs7761801 | ATTGGTGCAGACACCCTGCTTT | SEQ ID NO: 1920 | ATTGGTGCAGACACCCTGCTTC | SEQ ID NO: 4286 | AACCAGTGATGGGTGACAA | SEQ ID NO: 6652 |
| rs2077022 | CCGAGATCACGTCACTGCACTCTAC | SEQ ID NO: 1921 | CCGAGATCACGTCACTGCACTCTAG | SEQ ID NO: 4287 | ATGCCGTGTCAAAAAAAATAA | SEQ ID NO: 6653 |
| rs7743662 | ATGCCAGGGTTGCAGTGAGCTAGT | SEQ ID NO: 1922 | ATGCCAGGGTTGCAGTGAGCTAGC | SEQ ID NO: 4288 | TATAGATGTCTGATGATAGTTGGG | SEQ ID NO: 6654 |
| rs1264319 | GTGGGAATCCGCCCCACTCT | SEQ ID NO: 1923 | GTGGGAATCCGCCCCACTCC | SEQ ID NO: 4269 | AATGGCTCTGGTAAGACCTG | SEQ ID NO: 6655 |
| rs204990 | CAATCTTCCTACCTCAGCCTCTCATTT | SEQ ID NO: 1924 | CAATCTTCCTACCTCAGCCTCTCATTG | SEQ ID NO: 4290 | TCCATATCTATATCTCTTATGCCC | SEQ ID NO: 6656 |
| rs2736158 | CTTCCAGCCTCCGCTCCTGG | SEQ ID NO: 1925 | CTTCCAGCCTCCGCTCCTGC | SEQ ID NO: 4291 | CCTCACAACAGTCACAGTGG | SEQ ID NO: 6657 |
| rs13409 | GATAAGTGTGTCTATCTACTGTGTCCCAA | SEQ ID NO: 1926 | GATAAGTGTGTCTATCTACTGTGTCCCAG | SEQ ID NO: 4292 | CTTCTTTATTTAAGAAAAAAGTGAT | SEQ ID NO: 6658 |
| rs3130057 | GGGGGACATTAAATTTTTTAAATTAGGTGA | SEQ ID NO: 1927 | GGGGGACATAAAATTTTTAAATTAGGTGC | SEQ ID NO: 4293 | CTCTAAAGTTAGTTTTAGTTTGGA | SEQ ID NO: 6659 |
| rs2857127 | GCTTCTTGGTGTTCTCAACAATTTTAT | SEQ ID NO: 1928 | GCTTCTTGGTGTTCTCAACAATTTTAC | SEQ ID NO: 4294 | CAAAAGAATTATAAGCTACTGATGT | SEQ ID NO: 6660 |
| rs7887 | AGGCTCCAGGGAGTCGGGGT | SEQ ID NO: 1929 | AGGCTCCAGGGAGTCGGGGG | SEQ ID NO: 4295 | GTTTCTTCACTACGAGGGGT | SEQ ID NO: 6661 |
| rs2535277 | CCTCAGTGTCTTCCCTGTGCAGTA | SEQ ID NO: 1930 | CCTCAGTGTCTTCCCTGTGCAGTG | SEQ ID NO: 4296 | GGGGTTGTTGTGAGGATTT | SEQ ID NO: 6662 |
| rs2395296 | TCTTCTTATACAGCTGTTACACATGCACA | SEQ ID NO: 1931 | TCTTCTTATACAGCTGTTACACATGCACG | SEQ ID NO: 4297 | CATACACACTCTCTTGAGCATG | SEQ ID NO: 6663 |
| rs2294583 | TGTCTCTAAGATTGTTTCTTAGCTGTAAA | SEQ ID NO: 1932 | TGTCTCTAAGATTGTTTCTTAGCTGTAAT | SEQ ID NO: 4298 | TGAGGATAAAGCAATTAAACTTT | SEQ ID NO: 6664 |
| rs3129681 | GGAAGTACAGAAACCTATAGAAGGGTATCA | SEQ ID NO: 1933 | GGAAGTACAGAAACCTATAGAAGGGTATCG | SEQ ID NO: 4299 | AATAGGGTCTAACTTAATTTGAGAT | SEQ ID NO: 6665 |
| rs3130141 | CCAAGAGGACGCAGCACTGGA | SEQ ID NO: 1934 | CCAAGAGGACGCAGCACTGGC | SEQ ID NO: 4300 | GCTTCAGGAGGTGGCTGT | SEQ ID NO: 6666 |
| rs2285042 | GCACGGATGTCTGCCTCCTGT | SEQ ID NO: 1935 | GCACGGATGTCTGCCTCCTGC | SEQ ID NO: 4301 | AGTGTGTGCCCCAGTTTAC | SEQ ID NO: 6667 |
| rs5027663 | TTCTCCATGTCCTTGGCTATATTTGT | SEQ ID NO: 1936 | TTCTCCATGTCCTTGGCTATATTTGA | SEQ ID NO: 4302 | ATTCCAGACTTAAACTTAAGATATA | SEQ ID NO: 6668 |
| rs2294478 | TCCCTGGTCCCTTCATTTTCTGA | SEQ ID NO: 1937 | TCCCTGGTCCCTTCATTTTCTGC | SEQ ID NO: 4303 | CCTCACAGTGTGAATAAACT | SEQ ID NO: 6669 |
| rs6457710 | TCCTTTTCTTTTAATGTCATTGTTTGA | SEQ ID NO: 1938 | TCCTTTTCTTTTAATGTCATTGTTTGG | SEQ ID NO: 4304 | AAATTATTGGGATGTGTTTTCTT | SEQ ID NO: 6670 |
| rs204900 | CATACCTGTGTTGGCCCTGACAGA | SEQ ID NO: 1939 | CATACCTGTGTTGGCCCTGACAGC | SEQ ID NO: 4305 | GCTGGGTAGCTGACTGC | SEQ ID NO: 6671 |
| rs5018660 | GCATTTAATAATAGCATCATCTTCACAGGA | SEQ ID NO: 1940 | GCAAAAATAATAGCATCATCTTCACAGGG | SEQ ID NO: 4306 | AATAATGACTTCTTAGCACTGACC | SEQ ID NO: 6672 |
| rs6906587 | CCGCATCTCCTGGAGTTGGC | SEQ ID NO: 1941 | CCGCATCTCCTGGAGTTGGG | SEQ ID NO: 4307 | GAGCTAGTGCTTCCTGCTG | SEQ ID NO: 6673 |
| rs2242653 | GGAAGGGAAGGGTCCCGTGAA | SEQ ID NO: 1942 | GGAAGGGAAGGGTCCCGTGAG | SEQ ID NO: 4308 | GGCCGTGTTCAGTCCTTCT | SEQ ID NO: 6674 |
| rs444921 | TTGTTGCCCACTTAGGGGCTGT | SEQ ID NO: 1943 | TTGTTGCCCACTTAGGGGCTGC | SEQ ID NO: 4309 | CAGAGGGGCAGAGGTTTAG | SEQ ID NO:.6675 |
| rs2187820 | TGTTCTGGGTACTAATCCTTTTCATA | SEQ ID NO: 1944 | TGTTCTGGGTACTAATCCTTTTCATG | SEQ ID NO: 4310 | TCTTTATCCAGTTTACCAAAAAA | SEQ ID NO: 6676 |
| rs2071281 | CACTGCCCACCGGGCTTT | SEQ ID NO: 1945 | CACTGCCCACCGGGCTTC | SEQ ID NO: 4311 | GTGATGTCAACGAGTGCTTC | SEQ ID NO: 6677 |
| rs3116820 | ACCCTAAGTGGTCTCAATTGTTTACTAA | SEQ ID NO: 1946 | ACCCTAAGTGGTCTCAATTGTTTACTAG | SEQ ID NO: 4312 | AGTTATTTTACATACACTATTCTTGG | SEQ ID NO: 6678 |
| rs241407 | CGGAGTCTCGGAACGCCTGT | SEQ ID NO: 1947 | CGGAGTCTCGGAACGCCTGC | SEQ ID NO: 4313 | TGGAAGAAGAGTTCCGGC | SEQ ID NO: 6679 |
| rs6457443 | AAGCAATGGCAATTAAAAGTAGAGTAA | SEQ ID NO: 1948 | AAGCAATGGCAATTAAAAGTAGAGTAG | SEQ ID NO: 4314 | AGTAAATAACCAGTACTAAGTTCTTCC | SEQ ID NO: 6680 |
| rs668558 | TGCCACCTTCTGTACGAGTTTGTTTT | SEQ ID NO: 1949 | TGCCACCTTCTGTACGAGTTTGAAG | SEQ ID NO: 4315 | TTGCTTGGCGAAAACACTAC | SEQ ID NO: 6681 |
| rs2516675 | GAGAATGGATACTTGAGGATCAGTATAGTT | SEQ ID NO: 1950 | GAGAATGGATACTTGAGGATCAGTATAGTC | SEQ ID NO: 4316 | CTAGATGAAGAGGACTACATACATTT | SEQ ID NO: 6682 |
| rs909726 | CAAAGGGTCTCATTCAGCTCACCT | SEQ ID NO: 1951 | CAAAGGGTCTCATTCAGCTCACCC | SEQ ID NO: 4317 | ACTTTTAATACTTAAATAACGACAAT | SEQ ID NO: 6683 |
| rs4842463 | TGATTGAGATGATATTTTTAAACAAAAAAA | SEQ ID NO: 1952 | TGATTGAGATGATATTTTTAAACAAAAAAG | SEQ ID NO: 4318 | TTTTGAAAATTAAGAACAGAAAAC | SEQ ID NO: 6684 |
| rs3020644 | AGAAGTCTTTTCAATACCTTTTTGAACAAT | SEQ ID NO: 1953 | AGAAGTCTTTTCAATACCTTTTTGAACAAC | SEQ ID NO: 4319 | GACCAATAAATAAATGAACATGA | SEQ ID NO: 6685 |
| rs2260801 | GGCAGTGGCTGCGATTCCA | SEQ ID NO: 1954 | GGCAGTGGCTGCGATTCCG | SEQ ID NO: 4320 | GGAACTGAGGGGACAGAAT | SEQ ID NO: 6666 |
| rs1736976 | TGCACTAGTTCCTAACCTATGTAACCTACT | SEQ ID NO: 1955 | TGCACTAGTTCCTAACCTATGTAACCTACC | SEQ ID NO: 4321 | TCTATGATCAAGAGTAGATTAAGAGA | SEQ ID NO: 6687 |
| rs1362065 | AAATAACATTTATTGCATGCTCATTA | SEQ ID NO: 1956 | AAATAACATTTATTGCATGCTCATTG | SEQ ID NO: 4322 | GTATAAGTTATTGTTCTGAGTTCTTCA | SEQ ID NO: 6688 |
| rs3094188 | TCAATGAACTCTAAACTTCCTTCCAACTA | SEQ ID NO: 1957 | TCAATGAACTCTAAACTTCCTTCCAACTC | SEQ ID NO: 4323 | GACATAAAATTGCTGCCC | SEQ ID NO: 6689 |
| rs7764777 | ACCGCAACCGGGTTCCTGA | SEQ ID NO: 1958 | ACCGCAACCGGGTTCCTGC | SEQ ID NO: 4324 | CCTAGCTACTCCCCACAGA | SEQ ID NO: 6690 |
| rs4959117 | TCTTAAGTCTAATTAAGCCAAACAAACTAT | SEQ ID NO: 1959 | TCTTAAGTCTAATTAAGCCAAACAAACTAA | SEQ ID NO: 4325 | AATAGCAATTTAATTAGCAAGCTA | SEQ ID NO: 6691 |
| rs2844621 | AGGACACTTCAAATGGTGTAGTTTCAT | SEQ ID NO: 1960 | AGGACACTTCAAATGGTGTAGTTTCAC | SEQ ID NO: 4326 | TAAACTATCATTATGCTATTATTATTG | SEQ ID NO: 6692 |
| rs3099840 | CTGAAGCCCAGAGAGCCAAGCT | SEQ ID NO: 1961 | CTGAAGCCCAGAGAGCCAAGCC | SEQ ID NO: 4327 | CTGGGACTTGGCTCTTACC | SEQ ID NO: 6693 |
| rs2073525 | GCGGGGCGGGCCTACTGT | SEQ ID NO: 1962 | GCGGGGCGGGCCTACTGC | SEQ ID NO: 4328 | CTGTGTTCCGTACGACAATAT | SEQ ID NO: 6694 |
| rs3094693 | CTTCTCTCCGAGGCTCGCGGA | SEQ ID NO: 1963 | CTTCTCTCCGAGGCTCGCGGC | SEQ ID NO: 4329 | TGAGAAACCTTCCGCTCC | SEQ ID NO: 6695 |
| rs7759272 | GATGAATAGTTGCACTTTAATTTTCAT | SEQ ID NO: 1964 | GATGAATAGTTGCAGTTTAATTTTCAC | SEQ ID NO: 4330 | TGCTCTAAATTTATTAGTGTTATTGT | SEQ ID NO: 6696 |
| rs6911350 | TCTTTCTAGTGCTGTGTCTCATTGTGAG | SEQ ID NO: 1965 | TCTTTCTAGTGCTGTGTCTCATTGTGAC | SEQ ID NO: 4331 | TATATATTTCTTTCTAGGACTTGAAA | SEQ ID NO: 6697 |
| rs2070600 | GTGGCTCGTGTCCTTCCCAACA | SEQ ID NO: 1966 | GTGGCTCGTGTCCTTCCCAACG | SEQ ID NO: 4332 | CTCCCTCTTCCTTCCGG | SEQ ID NO: 6698 |
| rs2269428 | CTCCCACCCCTGAACCCCA | SEQ ID NO: 1967 | CTCCCACCCCTGAACCCCC | SEQ ID NO: 4333 | GAGCTGACCGTGACAGATG | SEQ ID NO: 6699 |
| rs2302399 | ATCAGTCAGGAGGCCGTGCAGT | SEQ ID NO: 1968 | ATCAGTCAGGAGGCCGTGCAGC | SEQ ID NO: 4334 | CGAGATGGGCTCGTCTC | SEQ ID NO: 6700 |
| rs1633098 | TGGTAGTTTGTTATAGAGGTTTGCGTA | SEQ ID NO: 1969 | TGGTAGTTTGTTATAGAGGTTTGCGTC | SEQ ID NO: 4335 | TTTTATTTTGCCATGAGGAT | SEQ ID NO: 6701 |
| rs555007 | GGCTGCTCAGCTGCAGGAAT | SEQ ID NO: 1970 | GGCTGCTCAGCTGCAGGTAC | SEQ ID NO: 4336 | GAGAGTGGCCAGATGGAGAC | SEQ ID NO: 6702 |
| rs2508042 | TGTTCTCAACTGTGTTAACTTATTGAAA | SEQ ID NO: 1971 | TGTTCTCAACTGTGTTAACTTATTGAAG | SEQ ID NO: 4337 | ACATTGTTATTAAAGGTATTTACAAGA | SEQ ID NO: 6703 |
| rs3130653 | TTTCTTTAGTTAGATTAGGCTAGGCTGTT | SEQ ID NO: 1972 | TTTCTTTAGTTAGATTAGGCTAGGCTGTG | SEQ ID NO: 4338 | AAACAAAATTTCAATAACTGGCT | SEQ ID NO: 6704 |
| rs1705003 | CAGAGCCAGCCAATCCCGA | SEQ ID NO: 1973 | CAGAGCCAGCCAATCCCGG | SEQ ID NO: 4339 | CACCTCCTGATGAAGGAGG | SEQ ID NO: 6705 |
| rs764554 | CATTTACCAGGAGAAACCATATTTCAGT | SEQ ID NO: 1974 | CATTTACCAGGAGAAACCATATTTCAGG | SEQ ID NO: 4340 | ACATTGAAATCATACAGAATATATTTT | SEQ ID NO: 6706 |
| rs2532942 | ATCTCGGTTGCCCCCTACCCT | SEQ ID NO: 1975 | ATCTCGGTTGCCCCCTACCCC | SEQ ID NO: 4341 | GTGAGTCCCAAGCACCTT | SEQ ID NO: 6707 |
| rs213207 | CACAACTAACTACACATTCTGACTTAAGGA | SEQ ID NO: 1976 | CACAACTAACTACACATTCTGACTTAAGGG | SEQ ID NO: 4342 | AAATAAAAGAACGTTATGTATCTA | SEQ ID NO: 6708 |
| rs2428494 | GGGGGTGGGTGGGGCAGA | SEQ ID NO: 1977 | GGGGGTGGGTGGGGCAGT | SEQ ID NO: 4343 | GATTCTTTGATTGGGATGAAC | SEQ ID NO: 6709 |
| rs1799910 | CTGCACTCCATCTCGGCCAGTT | SEQ ID NO: 1978 | CTGCACTCCATCTCGGCCAGTC | SEQ ID NO: 4344 | CTTCTCACCCTGTGGGAC | SEQ ID NO: 6710 |
| rs2071480 | CTTTGCGCGCGGCGCTAACT | SEQ ID NO: 1979 | CTTTGCGCGCGGCGCTAACG | SEQ ID NO: 4345 | GTGTAGGGCAGATCTGCC | SEQ ID NO: 6711 |
| rs1079687 | GGCCCCTCAGTGAGGGGT | SEQ ID NO: 1980 | GGCCCCTCAGTGAGGGGC | SEQ ID NO: 4346 | AAGAGTCCAGCTCTTTCCTTT | SEQ ID NO: 6712 |
| rs970902 | AGGGGCTGAGTGGGGGAAT | SEQ ID NO: 1981 | AGGGGCTGAGTGGGGGAAC | SEQ ID NO: 4347 | CAGCTTCCTTCCTGGGGT | SEQ ID NO: 6713 |
| rs6900438 | CAGACATGAGTTACTGCGCCCCA | SEQ ID NO: 1982 | CAGACATGAGTTACTGCGCCCCG | SEQ ID NO: 4348 | CTGAAATTCTTTTTTTAATTTAAAA | SEQ ID NO: 6714 |
| rs213213 | GGGAGAGGCCCTTGCCACA | SEQ ID NO: 1983 | GGGAGAGGCCCTTGCCACG | SEQ ID NO: 4349 | AGTGATTGGGTTCAGGCAG | SEQ ID NO: 6715 |
| rs410909 | CTGTCCCACAACGCTCACTCAGT | SEQ ID NO: 1984 | CTGTCCCACAACGCTCACTCAGG | SEQ ID NO: 4350 | GTGTGTGTGTGCCTGTGTGT | SEQ ID NO: 6716 |
| rs1052486 | GGCCAGGGGCTGGAGGGT | SEQ ID NO: 1985 | GGCCAGGGGCTGGAGGGC | SEQ ID NO: 4351 | CTTCTCCCACCATCACTGT | SEQ ID NO: 6717 |
| rs1150738 | AGTTGTTGGTGGAAGCGGGAGA | SEQ ID NO: 1986 | AGTTGTTGGTGGAAGCGGGAGG | SEQ ID NO: 4352 | GAATGGCACGTCTGGAG | SEQ ID NO: 6718 |
| rs130076 | GAGGTCTCCCGCAGGAGCCA | SEQ ID NO: 1987 | GAGGTCTCCCGCAGGAGCCG | SEQ ID NO: 4353 | ACCTCCTCCTCCAGCC | SEQ ID NO: 6719 |
| rs2395174 | CCTCCCAAATCTCATAATACTGAGTTTCA | SEQ ID NO: 1988 | CCTCCCAAATCTCATAATACTGAGTTTCC | SEQ ID NO: 4354 | GTTCAGAAACCTGGGGCAG | SEQ ID NO: 6720 |
| rs2252223 | GACCCCCGGAAACGTCCCA | SEQ ID NO: 1989 | GACCCCCGGAAACGTCCCG | SEQ ID NO: 4355 | GAGGACCGCCAGGAAC | SEQ ID NO: 6721 |
| rs1264303 | CACTCCCCTTGTTCCATCTTTCCT | SEQ ID NO: 1990 | CACTCCCCTTGTTCCATCTTTCCC | SEQ ID NO: 4356 | TCTCTAATTAGCACAGGTCTGTT | SEQ ID NO: 6722 |
| rs2240061 | CCCArCAAGAAGAGCCTCACGAT | SEQ ID NO: 1991 | CCCATCAAGAAGAGCCTCACGAC | SEQ ID NO: 4357 | ACAATAAACATTTACAAGCCAGA | SEQ ID NO: 6723 |
| rs1345229 | TTCTTCTGAATGTCCCACCCACTT | SEQ ID NO: 1992 | TTCTTOTGAATGTCCCACCCACTC | SEQ ID NO: 4358 | ACTATACATTGCTTAAATGCAGTG | SEQ ID NO: 6724 |
| rs2395185 | ACAGCAGAATTCTCCAGGGAAGAA | SEQ ID NO: 1993 | ACAGCAGAATTCTCCAGGGAAGAC | SEQ ID NO: 4359 | AATTTTTGGTTTCATGTAGTGTA | SEQ ID NO: 6725 |
| rs3130283 | GGTAGAACTGCCCTCATAAGCAGGT | SEQ ID NO: 1994 | GGTAGAACTGCCCTCATAAGCAGGG | SEQ ID NO: 4360 | ACATATACTTTTGGTCACCCTTT | SEQ ID NO: 6726 |
| rs2257914 | TGACATCTGAGAAATGGGATGGAAT | SEQ ID NO: 1995 | TGACATCTGAGAAATGGGATGGAAG | SEQ ID NO: 4361 | GTGACCATGTTTTATTTTCTTGT | SEQ ID NO: 6727 |
| rs916920 | GGATGGGAGCTCCCTGTACAGGA | SEQ ID NO: 1996 | GGATGGGAGCTCCCTGTACAGGG | SEQ ID NO: 4362 | CAGAGGTCAAAGGTTAGTATCAG | SEQ ID NO: 6728 |
| rs2249059 | CAAGCCCTGTTCTTCCTCTCCCA | SEQ ID NO: 1997 | CAAGCCCTGTTCTTCCTCTCCCG | SEQ ID NO: 4363 | TGAATTCTAAATCCTTAACATCCA | SEQ ID NO: 6729 |
| rs2267640 | CCATACTCTACTGGCACAAGGCAT | SEQ ID NO: 1998 | CCATACTCTACTGGCACAAGGCAC | SEQ ID NO: 4364 | GGGGAATTTTCTACCATTTTTA | SEQ ID NO: 6730 |
| rs1150765 | AGTATGGGAGGCAGGCAGCAA | SEQ ID NO: 1999 | AGTATGGGAGGCAGGCAGCAG | SEQ ID NO: 4365 | GTCTTGTGGAGACCTTACTCATT | SEQ ID NO: 6731 |
| rs3749967 | AACAGCTTTGGGTATAGTCTCGGGT | SEQ ID NO: 2000 | AACAGCTTTGGGTATAGTCTCGGGG | SEQ ID NO: 4366 | GAGACTGCCATATCTTTCTGTTT | SEQ ID NO: 6732 |
| rs3817963 | CTTAAGCACACCTTTCAGCCCACA | SEQ ID NO: 2001 | CTTAAGCACACCTTTCAGCCCACG | SEQ ID NO: 4367 | AAGAGTTTTTGAGGAAAATCAG | SEQ ID NO: 6733 |
| rs2051549 | CCATGGAGCTCTTGGGAATCTCA | SEQ ID NO: 2002 | CCATGGAGCTCTTGGGAATCTCG | SEQ ID NO: 4368 | AGACAGAGTTATCCACATAAATTTG | SEQ ID NO: 6734 |
| rs1894408 | TGCACAGTATAGGCAAATCAGGGG | SEQ ID NO: 2003 | TGCACAGTATAGGCAAATCAGGGC | SEQ ID NO: 4369 | ACATTTCATGCTGAAAGATGAT | SEQ ID NO: 6735 |
| rs3130315 | GCTGCAAAGATATTGGGTTTTGGTTA | SEQ ID NO: 2004 | GCTGCAAAGATATTGGGTTTTGGTTG | SEQ ID NO: 4370 | AGAGTGAGATGCTGATGTGTAAG | SEQ ID NO: 6736 |
| rs761187 | CAAAATTCACTTGTGCCCTATGGAGA | SEQ ID NO: 2005 | CAAAATTCACTTGTGCCCTATGGAGG | SEQ ID NO: 4371 | GAAGAATAGCAAGAGACCAAATC | SEQ ID NO: 6737 |
| rs3749966 | GGGCTCCCTGTCCTTGTGGTAT | SEQ ID NO: 2006 | GGGCTCCCTGTCCTTGTGGTAC | SEQ ID NO: 4372 | CTCATCTCACTGATTTTTAGTTGG | SEQ ID NO: 6738 |
| rs2395153 | TGGTTGGACCCAGTAACCCAGAC | SEQ ID NO: 2007 | TGGTTGGACCCAGTAACCCAGAG | SEQ ID NO: 4373 | GGGGACAGACTTTTCTTGTTAG | SEQ ID NO: 6739 |
| rs1150735 | CAAAGCCAGCAATGTCAGGCT | SEQ ID NO: 2008 | CAAAGCCAGCAATGTCAGGCC | SEQ ID NO: 4374 | ATCACTTCTCATGCTGCTATCT | SEQ ID NO: 6740 |
| rs7843 | GTCACATGCTGCCTGAGTCACAT | SEQ ID NO: 2009 | GTCACATGCTGCCTGAGTCACAC | SEQ ID NO: 4375 | TACATCTTATTTCTGCCTCTAGTTT | SEQ ID NO: 6741 |
| rs2074478 | CGTGAATTCTTCCCAATTTAGCCTA | SEQ ID NO: 2010 | CGTGAATTCTTCCCAATTTAGCCTG | SEQ ID NO: 4376 | CTCCTTAAACCTCTTCCTCATT | SEQ ID NO: 6742 |
| rs204993 | CAGGACATCAACATCCTCCTCACT | SEQ ID NO: 2011 | CAGGACATCAACATCCTCCTCACC | SEQ ID NO: 4377 | CTCTTCTCCCTCCTCTGTAGAC | SEQ ID NO: 6743 |
| rs2301271 | GGGGTTATATCGTCTTCCCTCCCA | SEQ ID NO: 2012 | GGGGTTATATCCTCTTCCCTCCCG | SEQ ID NO: 4378 | GTTCTTTAAGTCCTTAAGCACCC | SEQ ID NO: 674-4 |
| rs2284168 | CAGGGACACTTTATTGCTCACCCT | SEQ ID NO: 2013 | CAGGGACACTTTATTGCTCACCCC | SEQ ID NO: 4379 | TATGTGAAAGTTTGTCATAGCTAAG | SEQ ID NO: 6745 |
| rs11244 | CCCCTGAGACCTTACTCCTTCCAGT | SEQ ID NO: 2014 | CCCCTGAGACCTTACTCCTTCCAGC | SEQ ID NO: 4380 | CCAAATCATTTACTTTTCTGTG | SEQ ID NO: 6746 |
| rs1264583 | TGCAGGGTGTTTGTAAACAGCAGT | SEQ ID NO: 2015 | TGCAGGGTGTTTGTAAACAGCAGC | SEQ ID NO: 4381 | CAATTTACACCGGATATTAGG | SEQ ID NO: 6747 |
| rs2074474 | CGGATGTGACCCTGGACCCT | SEQ ID NO: 2016 | CGGATGTGACCCTGGACCCC | SEQ ID NO: 4382 | AGACAGCTCATCCTAACCTAGTC | SEQ ID NO: 6748 |
| rs2213571 | GGTTTAACCCAGGATCTCACTGAAAAC | SEQ ID NO: 2017 | GGTTTAACCCAGGATCTCACTGAAAAG | SEQ ID NO: 4383 | GATCCTACTACAGCAGTTTCTTG | SEQ ID NO: 6749 |
| rs204991 | CTGCCCCTTTGTCTGCACTAAACTA | SEQ ID NO: 2018 | CTGCCCCTTGTCTGCACTAAACTG | SEQ ID NO: 4384 | AAGTTTCCATATGAACCCTT | SEQ ID NO: 6750 |
| rs3129881 | TGGAAAATATACCTGGGGACTGAAA | SEQ ID NO: 2019 | TGGAAAATATACCTGGGGACTGAAG | SEQ ID NO: 4385 | ACTCCAAAAATTAAGACTGAGACC | SEQ ID NO: 6751 |
| rs1049623 | TCGACAGOCCTGAAGATCTGGTT | SEQ ID NO: 2020 | TCGACAGCCCTCAAGATCTGGTC | SEQ ID NO: 4386 | GTCTTGATTTCCCCCTTAATG | SEQ ID NO: 6752 |
| rs974962 | GATGGATTCACCCCATCTCCCA | SEQ ID NO: 2021 | GATGGATTCACCCCATCTCCCG | SEQ ID NO: 4387 | ATAAAGTTTACTTGTTTTACATTCCA | SEQ ID NO: 6753 |
| rs8512 | GCAAGGGTGTGAGATGTTCCCT | SEQ ID NO: 2022 | GCAAGGGTGTGAGATGTTCCCC | SEQ ID NO: 4388 | TGCTGTAAATGCAGGTCTCTT | SEQ ID NO: 6754 |
| rs2071287 | CTTGCATTATTCTTTCCCGCTCTCT | SEQ ID NO: 2023 | CTTGCATTATTCTTCCCGCTCTCC | SEQ ID NO: 4389 | TCAAGCAAACTCTTGGGTTAA | SEQ ID NO: 6755 |
| rs1058318 | GGACATTGGATTCATTTCCTCAAAATA | SEQ ID NO: 2024 | GGACATTGGATTCAAACCTCAAAATG | SEQ ID NO: 4390 | GGAGAGAAAGGAGAAAAATCAG | SEQ ID NO: 6756 |
| rs2269702 | GGGTGAGAGCTACTTTTTCTACCTCCTA | SEQ ID NO: 2025 | GGGTGAGAGCTACTTTTTCTACCTCCTG | SEQ ID NO: 4391 | TCCACAAGTCATCTCTATATCTTC | SEQ ID NO: 6757 |
| rs2286404 | AGGAGCAAGTGAGTCCTGTTGTAAT | SEQ ID NO: 2026 | AGGAGCTTTGTGAGTCCTGTTGTAAC | SEQ ID NO: 4392 | CTTTTAGACACTTTATATATCAAGGG | SEQ ID NO: 6758 |
| rs2269707 | ATCAGCTCCCAGCCCCACCT | SEQ ID NO: 2027 | ATCAGCTCCCAGCCCGACCC | SEQ ID NO: 4393 | CCAAAGTCCCAGGTTTACAAG | SEQ ID NO: 6759 |
| rs2269424 | GTCCCACTACCCTCATCCCCAT | SEQ ID NO: 2028 | GTCCGACTACCCTCATCCCCAC | SEQ ID NO: 4394 | TAGAAGTATCTTTCCAACCCTCT | SEQ ID NO: 6760 |
| rs2040409 | GCCAAAGACCAGCAGGAGCATA | SEQ ID NO: 2029 | GCCAAAGACCAGCAGGAGCATC | SEQ ID NO: 4395 | AAGCAATGAACAAGTTGACTTTA | SEQ ID NO: 6761 |
| rs241424 | GGGAGAAGCATACCCATCCTGAGA | SEQ ID NO: 2030 | GGGAGAAGCATACCCATCCTGAGG | SEQ ID NO: 4396 | AGTGACCCTGGAGAGATACTTT | SEQ ID NO: 6762 |
| rs2854275 | CGTGGTTCTGGCTCCACATTTA | SEQ ID NO: 2031 | CGTGGTTCTGGCTCCACATTTC | SEQ ID NO: 4397 | CAGGAAGATACCACCAAAGTTA | SEQ ID NO: 6763 |
| rs3880457 | GAGTCAGCCTCAGGGTCTGTTCTA | SEQ ID NO: 2032 | GAGTCAGCCTCAGGGTGTGTTCTG | SEQ ID NO: 4398 | GATCTCCTTTAGACCTTAACTGTT | SEQ ID NO: 6764 |
| rs4711206 | CTTTTCTCTCCACATCCCAGCCT | SEQ ID NO: 2033 | CTTTTCTCTCCACATCCCAGCCC | SEQ ID NO: 4399 | CTAATCCAGCTCTTACAATCTCA | SEQ ID NO: 6765 |
| rs1233495 | TGGGAAAGTTGGAGAACCATCCT | SEQ ID NO: 2034 | TGGGAAAGTTGGAGAACCATCCC | SEQ ID NO: 4400 | ATGGGCTAATGTAAACAAATGTA | SEQ ID NO: 6766 |
| rs3130055 | CCCTATTCGTTTTTGAACCGGTCA | SEQ ID NO: 2035 | CCCTATTCGTTTTTGAACCGGTCG | SEQ ID NO: 4401 | TTAAGGACACCTGTACTGAGAAG | SEQ ID NO: 6767 |
| rs2071593 | TTGGTAGCCTCGATGTCCTCCTT | SEQ ID NO: 2036 | TTGGTAGCCTCGATGTCCTCCTC | SEQ ID NO: 4402 | GTGAAACTGGGATGATAATAATG | SEQ ID NO: 6768 |
| rs7758397 | CCAGCCAGCAAGTCAATCAGAAC | SEQ ID NO: 2037 | CCAGGCAGCAAGTCAATCAGAAG | SEQ ID NO: 4403 | CTTCACCCCATCTACAATATTTT | SEQ ID NO: 6769 |
| rs3130453 | TCCAGGGGAACATAGATGGCTGA | SEQ ID NO: 2038 | TCCAGGGGAACATAGATGGCTGG | SEQ ID NO: 4404 | GACAGAATCTAGAGCCTTCAAAT | SEQ ID NO: 6770 |
| rs2242657 | CATCAGTCAGGTATTGGGGTGCTT | SEQ ID NO: 2039 | CATCAGTCAGGTATTGGGGTGCTC | SEQ ID NO: 4405 | CTACCCTGACTTAACTAGCTCCT | SEQ ID NO: 6771 |
| rs3130605 | ACTGTGCCTGCTTGCCATTAATCA | SEQ ID NO: 2040 | ACTGTGCCTGCTTGCCATTAATCG | SEQ ID NO: 4406 | AGATAAGGCACAACCCTATTACT | SEQ ID NO: 6772 |
| rs3093559 | GGAGGAGGCTGAAGGAGAAAGAAA | SEQ ID NO: 2041 | GGAGGAGGCTGAAGGAGAAAGAAG | SEQ ID NO: 4407 | GGAGAATATTAGAGAGAGATGGG | SEQ ID NO: 6773 |
| rs2235498 | AGGAAGCATCAGATGGGTGTCGA | SEQ ID NO: 2042 | AGGAAGCATCAGATGGGTGTCGG | SEQ ID NO: 4408 | GGAGACTGAAATAACAACACAAG | SEQ ID NO: 6774 |
| rs3135034 | CATTCAGTGCTGGAGACCAGCTA | SEQ ID NO: 2043 | CATTCAGTGCTGGAGACCAGCTG | SEQ ID NO: 4409 | TGGGATTTTGTGAGTATCTTTAG | SEQ ID NO: 6775 |
| rs3129952 | TTTTTCGCTTGGATCGGAAAGTG | SEQ ID NO: 2044 | TTTTTCGCTTGGATCGGAAAGTC | SEQ ID NO: 4410 | AAAATTTTTTTCCTGACTCAATT | SEQ ID NO: 6776 |
| rs6457602 | AATGCATCACACTAGCCCACAGG | SEQ ID NO: 2045 | AATGCATCACACAAGCCCACAGG | SEQ ID NO: 4411 | TTATTTACAACTCAGCTGGAAG | SEQ ID NO: 6777 |
| rs2523451 | TTCCCCCATGATCCCTAGCCTAT | SEQ ID NO: 2046 | TTCCCCCATGATCCCTAGCCTAC | SEQ ID NO: 4412 | CTTTTCTGTGGATGAAAATGTAA | SEQ ID NO: 6778 |
| rs497239 | GCCAGTCAGAACTGCCCTCAGA | SEQ ID NO: 2047 | GCCAGTCAGAACTGCCCTCAGG | SEQ ID NO: 4413 | AGACCACAGAGTCTACTCCTCTC | SEQ ID NO: 6779 |
| rs4959050 | CAGAGAATTGATAATTGCCCGGCA | SEQ ID NO: 2048 | CAGAGAATTGATAATTGCCCGGCG | SEQ ID NO: 4414 | TTTCATTTTCTTACAACCATGA | SEQ ID NO: 6780 |
| rs714289 | AGGTGATCCTCTTGCTTGCCTCTA | SEQ ID NO: 2049 | AGGTGATCCTCTTGCTTGCCTCTG | SEQ ID NO: 4415 | CTTGACATTTTTCAAACACAATC | SEQ ID NO: 6781 |
| rs447250 | ATGCTCCTGCCCTAAAGTGCCA | SEQ ID NO: 2050 | ATGCTCCTGCCCTAAAGTGCCG | SEQ ID NO: 4416 | TGCTATTGTTTGCAAGTTTAATT | SEQ ID NO: 6782 |
| rs2844775 | CAGGCTATTTGAGGTTGAGGCAAA | SEQ ID NO: 2051 | CAGGCTATTTGAGGTTGAGGCAAG | SEQ ID NO: 4417 | TCACATAAGTACGAGTGTCTCATT | SEQ ID NO: 6783 |
| rs3135002 | AAAAAAGGGAAAGCGGCAGCTA | SEQ ID NO: 2052 | AAAAAAGGGAAAGCGGCAGCTC | SEQ ID NO: 4418 | CTTGAATTTCTAAAGTGCAATGT | SEQ ID NO: 6784 |
| rs3077 | CAGCAATTCAGTCAGCCACTGGA | SEQ ID NO: 2053 | CAGCAATTCAGTCAGCCACTGGG | SEQ ID NO: 4419 | TAGTTTTCACATGAAGTGAGAAGA | SEQ ID NO: 6785 |
| rs3094205 | AAGGGACAGGCTGCTTGGATCA | SEQ ID NO: 2054 | AAGGGACAGGCTGCTTGGATCG | SEQ ID NO: 4420 | GATTGTAGATGACCTACAACACC | SEQ ID NO: 6786 |
| rs6927088 | CACTTCCAGAACTCCTCTTCCCAG | SEQ ID NO: 2055 | CACTTCCAGAACTCCTCTTCCCAC | SEQ ID NO: 4421 | CTGAAACTTCCTACCCACTAGAC | SEQ ID NO: 6787 |
| rs2857174 | TTGAGAAAAATGCATTTGTCCCGT | SEQ ID NO: 2056 | TTGAGAAAAATGCATTTGTCCCGC | SEQ ID NO: 4422 | GTTGGAGAATTCGTTAAGTTTGT | SEQ ID NO: 6788 |
| rs181997 | GCAGAGAAAAGCCTGCACTGGA | SEQ ID NO: 2057 | GCAGAGAAAAGCCTGCACTGGG | SEQ ID NO: 4423 | ATCTCTAGAAAAGGAACTGGAAG | SEQ ID NO: 6789 |
| rs261950 | GCAGCTGAGAGTGAGTTCACACCT | SEQ ID NO: 2058 | GCAGCTGAGAGTGAGTTCACACCA | SEQ ID NO: 4424 | GTACTCTAAAATGAGGAGGTGCT | SEQ ID NO: 6790 |
| rs3117329 | CCTGGTGATCTGGTTGCTTTGACA | SEQ ID NO: 2059 | CCTGGTGATCTGGTTGCTTTGACG | SEQ ID NO: 4425 | ATTCCTAGTGTCTGAAAATGAAAG | SEQ ID NO: 6791 |
| rs3128931 | ACTCTCCCTTTTTGCAGGGTCAT | SEQ ID NO: 2060 | ACTCTCCCTTTTTGCAGGGTCAC | SEQ ID NO: 4426 | CTACAAATCATAAGCATGTCATCT | SEQ ID NO: 6792 |
| rs6904456 | CTGTCTTCCCCCATTCTTCACCT | SEQ ID NO: 2061 | CTGTCTTCCCCCATTCTTCACCC | SEQ ID NO: 4427 | AATCCTAATTCTCCTTCAGAATC | SEQ ID NO: 6793 |
| rs4713216 | CCCCTATTAACGCTGACTTGGGA | SEQ ID NO: 2062 | CCCCTATTAACGCTGACTTGGGG | SEQ ID NO: 4428 | AGGAAAGGAAAGTTGTAACAGAA | SEQ ID NO: 6794 |
| rs3097671 | CAGAGTCACCAACCCCAGTACCAC | SEQ ID NO: 2063 | CAGAGTCACCAACCCCAGTACCAG | SEQ ID NO: 4429 | GTGCACAGCTAAGAAAATGAGTA | SEQ ID NO: 6795 |
| rs3135368 | TTTCCCAAGGTTAGGGACATGCT | SEQ ID NO: 2064 | TTTCCCAAGGTTAGGGACATGCA | SEQ ID NO: 4430 | GAAAGAAAAAATCATCAAATGAC | SEQ ID NO: 6796 |
| rs1264323 | CTGCTTGGTATTTCCCTGAGGCTA | SEQ ID NO: 2065 | CTGCTTGGTATTTCCCTGAGGCTG | SEQ ID NO: 4431 | TGAATTTGACCATTAAAAAAAAT | SEQ ID NO: 6797 |
| rs3094073 | CCTGGAAATAGGGTAAAAGCTGCAT | SEQ ID NO: 2066 | CCTGGAAATAGGGTAAAAGCTGCAC | SEQ ID NO: 4432 | GGCTTGAAAATTCATCTCTTAAT | SEQ ID NO: 6798 |
| rs2523995 | TCCTTGACCCCTCCACAATCTACA | SEQ ID NO: 2067 | TCCTTGACCCCTCCACAATCTACG | SEQ ID NO: 4433 | TCCTGTCGTATCTCATGAAATAG | SEQ ID NO: 6799 |
| rs3131635 | CCACAGTCAGGTTGTTTGATGGGA | SEQ ID NO: 2068 | CCACAGTCAGGTTGTTTGATGGGG | SEQ ID NO: 4434 | ATGAAAAGAGACAGCAGAAGTTT | SEQ ID NO: 6800 |
| rs1610724 | TCCTCCTCTTCTCACCTGCTTGTA | SEQ ID NO: 2069 | TCCTCCTCTTCTCACCTGCTTGTG | SEQ ID NO: 4435 | CTGGACACATACTATTCAAACATC | SEQ ID NO: 6801 |
| rs4713337 | CACCAGTTTTAAGTGCACAACCCAA | SEQ ID NO: 2070 | CACCAGTTTTAAGTGCACAACCCAG | SEQ ID NO: 4436 | TCTCACAAAGTAGACACACTTGA | SEQ ID NO: 6802 |
| rs2855442 | AGGGGACTAGGGAACTCCCCTTA | SEQ ID NO: 2071 | AGGGGACTAGGGAACTCCCCTTG | SEQ ID NO: 4437 | AGGCTTGACTTTCTGTGTATGT | SEQ ID NO: 6803 |
| rs885915 | TGGCACAAGGTCCTATTTGGAGT | SEQ ID NO: 2072 | TGGCACAAGGTCCTATTTGGAGC | SEQ ID NO: 4438 | ATAACAGCAGCTTCTTAATGAAA | SEQ ID NO: 6804 |
| rs241440 | GCACTTGTCCCTCCTTGTGTGTTA | SEQ ID NO: 2073 | GCACTTGTCCCTCCTTGTGTGTTG | SEQ ID NO: 4439 | CTGTGTCACTTGTATCTGAGGA | SEQ ID NO: 6805 |
| rs879882 | GTCCCTTGAAGGGGAAGTAGGGA | SEQ ID NO: 2074 | GTCCCTTGAAGGGGAAGTAGGGG | SEQ ID NO: 4440 | CAACCCCTTAGTCTGTTAGATG | SEQ ID NO: 6806 |
| rs6937061 | CCTCATTTrCAGTTCTGGGATGGA | SEQ ID NO: 2075 | CCTCATTTTCAGTTCTGGGATGGG | SEQ ID NO: 4441 | ACTCTAATCTCTCTAAATCATGCC | SEQ ID NO: 6807 |
| rs2023372 | CCCTTCATGCTTATGTCTCCCCA | SEQ ID NO: 2076 | CCCTTCATGCTTATGTCTCCCCG | SEQ ID NO: 4442 | TCCTGCTTTATACAAGTAATGTGT | SEQ ID NO: 6808 |
| rs1610601 | TTTACAAGCTGCATCCCATCTCAT | SEQ ID NO: 2077 | TTTACAAGCTGCATCCCATCTCAG | SEQ ID NO: 4443 | TAAGGAGGTGATGTAACTGAGTT | SEQ ID NO: 6809 |
| rs29228 | CTTACGGCCATCTCGGACAAAAA | SEQ ID NO: 2078 | CTTACGCCCATCTCGGACAAAAG | SEQ ID NO: 4444 | AGAAATGTATGTCTCAGTTGTGTT | SEQ ID NO: 6810 |
| rs7454372 | GTTGAACATCATGGTTGCCGAT | SEQ ID NO: 2079 | GTTGAACATCATGGTTGCCGAC | SEQ ID NO: 4445 | GTCATAAATAACAGTTGGAACAAA | SEQ ID NO: 6811 |
| rs707937 | GAACACTGTGGGCCCTCAAGTTG | SEQ ID NO: 2050 | GAACACTGTGGGCCCTCAAGTTC | SEQ ID NO: 4446 | ATGGAAATATAAAGGGCAGATC | SEQ ID NO: 6812 |
| rs7341251 | GGGATAGATGAAAGGGGCCATCT | SEQ ID NO: 2081 | GGGATAGATGAAAGGGGCCATCC | SEQ ID NO: 4447 | GAAAGGAAAAGATATCTGCTTG | SEQ ID NO: 6813 |
| rs1800750 | TTTTTCCTGCATCCTGTCTGGAAA | SEQ ID NO: 2082 | TTTTTCCTGCATCCTGTCTGGAAG | SEQ ID NO: 4448 | TAGAAGGAAACAGACCACAGAC | SEQ ID NO: 6814 |
| rs2394518 | CCCTCATGGTCATGAGCTCCATT | SEQ ID NO: 2083 | CCCTCATGGTCATGAGCTCCATA | SEQ ID NO: 4449 | TTGTTCTCATACCTCTCATCCT | SEQ ID NO: 6815 |
| rs2301225 | GCAATGTTCACACAAAAAATGCCT | SEQ ID NO: 2084 | GCAATGTTCACACAATTAAATGCCC | SEQ ID NO: 4450 | TCCAGAGATCTTACAGGTTTCTA | SEQ ID NO: 6816 |
| rs1264459 | TGGAAACGGGATTACAATGAAGCA | SEQ ID NO: 2085 | TGGAAACGGGATTACAATGAAGCG | SEQ ID NO: 4451 | GAGGTCTTTTTGAAGTCATG | SEQ ID NO: 6817 |
| rs7773955 | GGCACTGATGGTACCTCATTAGGCA | SEQ ID NO: 2086 | GGCACTGATGGTACCTCATTAGGCG | SEQ ID NO: 4452 | TTGTGAGGAATGTACATATACTGC | SEQ ID NO: 6818 |
| rs15912 | TCTTCTGTGTCACACACAGCCGTG | SEQ ID NO: 2087 | TCTTCTGTGTCACACACAGCCCTC | SEQ ID NO: 4453 | ATACTCCTCCACCTGAGATACC | SEQ ID NO: 6819 |
| rs1419675 | CAGTTGCCATACTGAACAGCCATTT | SEQ ID NO: 2088 | CAGTTGCCATACTGAACAGCCATTG | SEQ ID NO: 4454 | ATACAGGCTCTCTATATTCCAATG | SEQ ID NO: 6820 |
| rs7450074 | CTGCTTGCTCTAAACCCAGCAAT | SEQ ID NO: 2089 | CTGCTTGCTCTAAACCCAGCTAC | SEQ ID NO: 4455 | AGTTACTCCCTGCTTGGATAAT | SEQ ID NO: 6821 |
| rs437179 | CCAAATCCAGAGGCTCCAACAT | SEQ ID NO: 2090 | CCAAATCCAGAGGCTCCAACAG | SEQ ID NO: 4456 | CAGCTAAGGCTTAGTAGTCCAG | SEQ ID NO: 6822 |
| rs3132486 | TCCTTGTTGCACTGGTTAGGATGA | SEQ ID NO: 209T | TCCTTGTTGCACTGGTTAGGATGG | SEQ ID NO: 4457 | TGTTAGGTGTTTAAACAAGAATGA | SEQ ID NO: 6823 |
| rs2071286 | GAGTGGGGTGGGGTGAAATGT | SEQ ID NO: 2092 | GAGTGGGGTGGGGTGAAATGC | SEQ ID NO: 4458 | TTGAGTGTAGCTGGATTAAGTGT | SEQ ID NO: 6824 |
| rs2395029 | GACACATACTGTCCAATTCCCCTGT | SEQ ID NO: 2093 | GACACATACTGTCCAATTCCCCTGG | SEQ ID NO: 4459 | GCAGCTGTAATGTGTAGTTCAAT | SEQ ID NO: 6825 |
| rs7765791 | TGCAGTTCACTGACTCTCCTGCATA | SEQ ID NO: 2094 | TGCAGTTCACTGACTCTCCTGCATG | SEQ ID NO: 4460 | CATAGCCCTTATATTTGCTCTTC | SEQ ID NO: 6826 |
| rs3094576 | AAAGTCTCCCAGCTGCTCCTTCTT | SEQ ID NO: 2095 | TAAGTCTCCCAGCTGCTCCTTCTG | SEQ ID NO: 4461 | ATCTCCTTTATTGGTTTCTCCTC | SEQ ID NO: 6827 |
| rs3819269 | AGCTCAGACCTTGGCCATGAACA | SEQ ID NO: 2096 | AGCTCAGACCTTGGCCATGAACG | SEQ ID NO: 4462 | CAGGAATTTCTTGAAGGAAGAT | SEQ ID NO: 6828 |
| rs2074471 | CTGCACAATAGAAGCCCCCCTT | SEQ ID NO: 2097 | CTGCACAATAGAAGCCCCCCTC | SEQ ID NO: 4463 | CTCTTTGCACTTTCTAATCAGC | SEQ ID NO: 6829 |
| rs6929716 | AGTGAGGCCAGATTTTCCTCTTGTA | SEQ ID NO: 2098 | AGTGAGGCCAGATTTTCCTCTTGTG | SEQ ID NO: 4464 | TTTAGCCAAAACAACATATTGTT | SEQ ID NO: 6830 |
| rs2523608 | CTCGTGTTTTCCTGATCCTGCCT | SEQ ID NO: 2099 | CTCGTGTTTCCTGATCCTGCCC | SEQ ID NO: 4465 | GGGTCTGTAGTCATACTTCTGG | SEQ ID NO: 6831 |
| rs1264540 | CGCTTCACCCCAGAAGCAGTG | SEQ ID NO: 2100 | CGCTTCACCCCAGAAGCAGTC | SEQ ID NO: 4466 | GACTCCTATTCTTCAATACCAGG | SEQ ID NO: 6832 |
| rs3116996 | AACAATGGGGAGGCGTTTCTCT | SEQ ID NO: 2101 | AACAATGGGGAGGCGTTTCTCA | SEQ ID NO: 4467 | TGTATGAATTATTAGCTCAGGAGG | SEQ ID NO: 6833 |
| rs3130933 | GAGCATTGGATATTCCCATCCCT | SEQ ID NO: 2102 | GAGOATTGGATATTCCCATCCCC | SEQ ID NO: 4468 | CCTCAGTAACTGAAGGACAAAC | SEQ ID NO: 6834 |
| rs6940867 | AGTTGTGTGGCAGAAGGGGAACT | SEQ ID NO: 2103 | AGTTGTGTGGCAGAAGGGGAACC | SEQ ID NO: 4469 | CTGTTTCCTAACTTCTGGATAAG | SEQ ID NO: 6835 |
| rs2535250 | GCAACTTGGGAGAGTGTGAGCTTCA | SEQ ID NO: 2104 | GCAACTTGGGAGAGTGTGAGCTTCG | SEQ ID NO: 4470 | AGTCAGACAGGTCTAGGCTATTAG | SEQ ID NO: 6836 |
| rs6903608 | CCGTTTGGATTCCCAGGAGACA | SEQ ID NO: 2105 | CCGTTTGGATTCCCAGGAGACG | SEQ ID NO: 4471 | CACAGATGAGACACAAGGTAAG | SEQ ID NO: 6837 |
| rs3117435 | TGAGAATGGTGGTTCTTCCATCACG | SEQ ID NO: 2106 | TGAGAATGGTGGTTCTTCCATCACC | SEQ ID NO: 4472 | TATAGCTATTTCCACATCCATTT | SEQ ID NO: 6838 |
| rs1264425 | GGGTCTTGGGTTAGAGCCAAAATGG | SEQ ID NO: 2107 | GGGTCTTGGGTTAGAGCCAAAATGC | SEQ ID NO: 4473 | TTTGAGGATGAGTATTTTTTTTTT | SEQ ID NO: 6839 |
| rs3873342 | CTACTGGCTGGGGGATCACCAA | SEQ ID NO: 2108 | CTAGTGGCTGGGGGATCACCAG | SEQ ID NO: 4474 | TGTTCTACCATGAACAGGTCTAC | SEQ ID NO: 6840 |
| rs1265059 | CCCACAGAACCACAGACTTTGAAC | SEQ ID NO: 2109 | CCCACAGAACCACAGATTTTGAAG | SEQ ID NO: 4475 | GTTGCTGAGTAAATAGCAGACC | SEQ ID NO: 6841 |
| rs6911634 | AAAAATGGAGGAGAAAACAACCCAA | SEQ ID NO: 2110 | AAAAATGGAGGAGAAAACAACCCAG | SEQ ID NO: 4476 | GCGTAGAAGGACTATACAGTACT | SEQ ID NO: 6842 |
| rs464921 | TTTGCCATCACTGCCATTAAGGTA | SEQ ID NO: 2TTT | TTTGCCATCACTGCCATTAAGGTG | SEQ ID NO: 4477 | GTGAAGTAGGGTAAGGAATAGGG | SEQ ID NO: 6843 |
| rs4713221 | ATAGTCAGCCCACCCGCTCAACT | SEQ ID NO: 2112 | ATAGTCAGCCCACCCGCTCAACA | SEQ ID NO: 4478 | AGCTTCCATAGTCTTGTCTATTTG | SEQ ID NO: 6844 |
| rs382544 | GGCCCCTATCACTTCCATATGTGT | SEQ ID NO: 2113 | GGCCCCTATCACTTCCATATGTGC | SEQ ID NO: 4479 | CTGGACACCTCTAAGATCATAGG | SEQ ID NO: 6845 |
| rs3130977 | GAAACTCCAAGTCTGGAATCAGCAA | SEQ ID NO: 2114 | GAAACTCCAAGTCTGGAATCAGCAG | SEQ ID NO: 4480 | AATGTATTACATTGACCAGAAAGG | SEQ ID NO: 6846 |
| rs2395352 | GTCTGTTGGGGCTTAGTGCATTTA | SEQ ID NO: 2115 | GTCTGTTGGGGCTTAGTGCATTTG | SEQ ID NO: 4481 | CCTTTCATTAAAGTCCAAAACA | SEQ ID NO: 6847 |
| rs984802 | GCTGTGCGACTGGGCAATATTTTT | SEQ ID NO: 2116 | GCTGTGCGACTGGGCAATATTTTG | SEQ ID NO: 4482 | TTCACTCTCTAGCCTCATTTTC | SEQ ID NO: 6848 |
| rs1049633 | CTGAGAATCTGGGGGTGAGGAA | SEQ ID NO: 2117 | CTGAGAATCTGGGGGTGAGGAG | SEQ ID NO: 4483 | ACAAGAAGGAGAGGAAAATGTT | SEQ ID NO: 6849 |
| rs6923005 | ACACTGGACCCATTTTGCAGATAGA | SEQ ID NO: 2118 | ACACTGGACCCATTTTGCAGATAGG | SEQ ID NO: 4484 | AAACTAGGGTGGGTCTAAGGTAT | SEQ ID NO: 6850 |
| rs2001181 | TGGGCCGTTCAGAGTGTCATCA | SEQ ID NO: 2119 | TGGGCCGTTCAGAGTGTCATCG | SEQ ID NO: 4485 | TTACCATGACTGACCTGAATTT | SEQ ID NO: 6851 |
| rs2071541 | CCCTAACTGCATCACCTTTTCCCTT | SEQ ID NO: 2120 | CCCTAACTGCATCACCTTTTCCCTC | SEQ ID NO: 4486 | AGCTTTTTAATTCCTATGAGGC | SEQ ID NO: 6852 |
| rs3807039 | TGACTTGGCCAAGTTACACCTCAGT | SEQ ID NO: 212T | TGACTTGGCCAAGTTACACCTCAGG | SEQ ID NO: 4487 | GTGAGGTCTGTAGCAGGATAGA | SEQ ID NO: 6853 |
| rs6918223 | TGAGGGTTGGAAAAGCTTGTCTGA | SEQ ID NO: 2122 | TGAGGGTTGGAAAAGCTTGTCTGC | SEQ ID NO: 4488 | TTAATGACAAACCCAGAGACTG | SEQ ID NO: 6854 |
| rs3130501 | CCCAGTCATCCTCAGTTTCTTTCAT | SEQ ID NO: 2123 | CCCAGTCATCCTCAGTTTCTTTCAC | SEQ ID NO: 4489 | TCCTACATCCTATCCTTAAGAAAC | SEQ ID NO: 6855 |
| rs3869092 | AGGGAGATCAGCTTGCTCACAGT | SEQ ID NO: 2124 | AGGGAGATCAGCTTGCTCACAGC | SEQ ID NO: 4490 | GGCCATCTTTTAATCCATATTT | SEQ ID NO: 6856 |
| rs1736920 | CCATGAGAGATGCAAAGTGCTTGAA | SEQ ID NO: 2125 | CCATGAGAGATGCAAAGTGCTTGAG | SEQ ID NO: 4491 | TTTCTGACTCTTCCTTTCAGAC | SEQ ID NO: 6857 |
| rs3823418 | CCGTAGGCACAAAAATGGTAAAGGA | SEQ ID NO: 2126 | CCGTAGGCACAAAAATGGTAAAGGG | SEQ ID NO: 4492 | TTCAAGAAGAAATGCGTAGAAC | SEQ ID NO: 6858 |
| rs2844802 | CAGGAGACTAAGACGTGTCCTTTGCT | SEQ ID NO: 2127 | CAGGAGACTAAGACGTGTCCTTTGCA | SEQ ID NO: 4493 | TGTAGACTCCATATCTTGCACTT | SEQ ID NO: 6859 |
| rs2394412 | GCCCCATCAAGCCGACTTCTACT | SEQ ID NO: 2128 | GCCCCATCAAGCCGACTTCTACC | SEQ ID NO: 4494 | GTGACTCATTACCCAGTCATGT | SEQ ID NO: 6860 |
| rs439844 | TAGATCTGGGGTTGGGGATGGA | SEQ ID NO: 2129 | AAGATCTGGGGTTGGGGATGGG | SEQ ID NO: 4495 | ACAGAGAGTATGAGGGACTTGA | SEQ ID NO: 6861 |
| rs3763309 | CCTCCCAACTCCTCCTGCAAAGA | SEQ ID NO: 2130 | CCTCCCAACTCCTCCTGCAAAGC | SEQ ID NO: 4496 | TTTCTTTACCACTGTTTTCTACAC | SEQ ID NO: 6862 |
| rs7383312 | CTCCAGTTCTTTGCAAATGACATGA | SEQ ID NO: 2131 | CTCCAGTTCTTTGCAAATGACATGG | SEQ ID NO: 4497 | GGCTGAATAGTACTCCACATACA | SEQ ID NO: 6863 |
| rs1633021 | AGCAGGGAACAGGAAACTCAGTCTT | SEQ ID NO: 2132 | AGCAGGGAACAGGAAACTCAGTCTC | SEQ ID NO: 4498 | ACGACTGTCAGAAACTGAATTC | SEQ ID NO: 6864 |
| rs1367727 | ACAGCCACTAGTCACATGTGGCTAT | SEQ ID NO: 2133 | ACAGCCACTAGTCACATGTGGCTAC | SEQ ID NO: 4499 | GAGAACTTAAAAATGTGGCTAGTG | SEQ ID NO: 6865 |
| rs2269423 | CAAGATAGGGGTGAAGAGGTGGAAA | SEQ ID NO: 2134 | CAAGATAGGGGTGAAGAGGTGGAAC | SEQ ID NO: 4500 | AGAGAAGGTTAAACCCTCACAG | SEQ ID NO: 6866 |
| rs29269 | CTTCAGGCCTACCTAGCCCAGGT | SEQ ID NO: 2135 | CTTCAGGCCTACCTAGCCCAGGC | SEQ ID NO: 4501 | GGGTTAAAGCAGATAAACGAAG | SEQ ID NO: 6867 |
| rs2894054 | TGCTCCTATACCCTGGTCAACATTA | SEQ ID NO: 2136 | TGCTCCTATACCCTGGTCAACATTG | SEQ ID NO: 4502 | GGTTGTCAATCCTTAATTTCTTT | SEQ ID NO: 6868 |
| rs2534674 | CTGGGACCATAGTCCCCTTCAATA | SEQ ID NO: 2137 | CTGGGACCATAGTCCCCTTCAATG | SEQ ID NO: 4503 | GACAGATCTACAAGGACTCTCTACA | SEQ ID NO: 6869 |
| rs6915177 | CGTCTCAGCCCGTGAAAAAAACTT | SEQ ID NO: 2138 | CGTCTCAGCCCGTGAAAAITAACTC | SEQ ID NO: 4504 | CAGGTGATCAGTAGTGAGTCATG | SEQ ID NO: 6870 |
| rs4713559 | CCTTGTGAATCCCATCCTGGAAT | SEQ ID NO: 2139 | CCTTGTGAATCCCATCCTGGAAG | SEQ ID NO: 4505 | GAAGGTAAGATTGAGATTTGTTAGA | SEQ ID NO: 6871 |
| rs7382836 | GCCTCCTTAATGCCAGTGGTAACAT | SEQ ID NO: 2140 | GCCTCCTTAATGCCAGTGGTAACAG | SEQ ID NO: 4506 | AATACAGCCCCAGAATATCATT | SEQ ID NO: 6872 |
| rs4071986 | GACTCAATCTTCCTTCCTCTTGCCT | SEQ ID NO: 2141 | GACTCAATCTTCCTTCCTCTTGCCG | SEQ ID NO: 4507 | AGTGTTCCTCACTTAGTCTTGG | SEQ ID NO: 6873 |
| rs735883 | GGGTCTCTGCCCTTGTCTTTGCT | SEQ ID NO: 2142 | GGGTCTCTGCCCTTGTCTTTGCC | SEQ ID NO: 4508 | CTTCTTCTATCTCTACTCCTTGGG | SEQ ID NO: 6874 |
| rs3793127 | CTGCTTCCTTCTTTACTTCCCTGGA | SEQ ID NO: 2143 | CTGCTTCGTTCTTTACTTCCCTGGG | SEQ ID NO: 4509 | GGCTGTTTCCTAATACCTTTTT | SEQ ID NO: 6875 |
| rs2301269 | CACACACCTACATCCTGCCAAGAT | SEQ ID NO: 2144 | CACACACCTACATCCTGCCAAGAA | SEQ ID NO: 4510 | ATATTACTTGCCCAATCTGTCC | SEQ ID NO: 6876 |
| rs2844685 | AGAACACAGGCTCTGCTGTCAAGT | SEQ ID NO: 2145 | AGAACACAGGCTCTGCTGTCAAGC | SEQ ID NO: 4511 | GACCAGAATTAGAGCCTAGCTC | SEQ ID NO: 6877 |
| rs6908943 | CAGATGCACCTATTTCATGGTTCAGA | SEQ ID NO: 2146 | CAGATGCACCTATTTCATGGTTCAGG | SEQ ID NO: 4512 | AGGATTTAAACAGCTGAAGAACT | SEQ ID NO: 6878 |
| rs7383578 | AGGAACAGGACCCCTGCCAAA | SEQ ID NO: 2147 | AGGAACAGGACCCCTGCCAAG | SEQ ID NO: 4513 | CTACTAAATCATAGAGCCACTGG | SEQ ID NO: 6879 |
| rs1633088 | CCTCCTCAGGAAGGTCGGGACA | SEQ ID NO: 2148 | CCTCCTCAGGTAGGTCGGGACG | SEQ ID NO: 4514 | CAGACCTGTGTGTTAAACATTG | SEQ ID NO: 6880 |
| rs3871247 | TGAGGAAAAGAGAATGATGGGAAAA | SEQ ID NO: 2149 | TGAGGAAAAGAGAATGATGGGAAAG | SEQ ID NO: 4515 | GTTATTTTCCAACAGGAGTCTTAC | SEQ ID NO: 6881 |
| rs461338 | TGCCTCTGAGTCATTGTGTTTTTCAA | SEQ ID NO: 2150 | TGCCTCTGAGTCATTGTGTTTTTCAG | SEQ ID NO: 4516 | GTGATTTCTTTTCTGTAGCTTTTT | SEQ ID NO: 6882 |
| rs3807031 | GTTCTGGCAGTTGACTTTCCTGACA | SEQ ID NO: 2151 | GTTCTGGCAGTTGACTTTCCTGACC | SEQ ID NO: 4517 | ACTCCTGAAATCTGATACAAAC | SEQ ID NO: 6883 |
| rs6911709 | ACGTTGAAGCAGCCTTGCATTCT | SEQ ID NO: 2152 | ACGTTGAAGCAGCCTTGCATTCC | SEQ ID NO: 4518 | GGGATAAATTTCACTTTGTCGT | SEQ ID NO: 6884 |
| rs2524005 | AGGGGGCTTGACCTTCAAGGAA | SEQ ID NO: 2153 | AGGGGGCTTGACCTTCAAGGAG | SEQ ID NO: 4519 | TATGGGAAAGCTTAATAAAGGG | SEQ ID NO: 6885 |
| rs1736991 | GCATTCTGTGGGGCTGTCTAAAAC | SEQ ID NO: 2154 | GCATTCTGTGGGGCTGTCTAAAAG | SEQ ID NO: 4520 | TAATTGCCTCTCTAAGATAAAAAGG | SEQ ID NO: 6886 |
| rs4713422 | AGGGGAATGGGGGTGCTTAGAC | SEQ ID NO: 2155 | AGGGGAATGGGGGTGCTTAGAG | SEQ ID NO: 4521 | AGAGGAGGGTGAATCTAACAAA | SEQ ID NO: 6887 |
| rs6921921 | TCATGTTTTTTTGGCCACATGTACA | SEQ ID NO: 2156 | TCATGTTTTTTTGGCCACATGTACG | SEQ ID NO: 4522 | CTTATTTTGTGAAATGTCTCTTCA | SEQ ID NO: 6888 |
| rs7453137 | TTTTAGTCCCTCTGCTTCCCTCTTT | SEQ ID NO: 2157 | TTTTAGTCCCTCTGCTTCCCTCTTC | SEQ ID NO: 4523 | AATAATTTGTCAGGCCTTTTAAA | SEQ ID NO: 6889 |
| rs1801823 | GAAGCCTTTGCTTGGCCCTTA | SEQ ID NO: 2158 | GAAGCCTTTGCTTGGCCCTTG | SEQ ID NO: 4524 | ATGTTAGGATATGGCCAAGAAT | SEQ ID NO: 6890 |
| rs498240 | GCAGGCACATCAGAATAGGAGAGAT | SEQ ID NO: 2159 | GCAGGCACATCAGAATAGGAGAGAC | SEQ ID NO: 4525 | GATGGCTATATTTTGTAAGCCC | SEQ ID NO: 6891 |
| rs6906730 | CCCTGTGAAAATGAGATTGCATTCT | SEQ ID NO: 2160 | CCCTGTGAAAATGAGATTGCATTCC | SEQ ID NO: 4526 | TAAATCAGAATAGCACATTCACC | SEQ ID NO: 6892 |
| rs2057726 | GTGTTTCCTGCACTCATCTTGTGAT | SEQ ID NO: 2161 | GTGTTTCCTGCACTCATCTTGTGAC | SEQ ID NO: 4527 | GAGTTCTATTTGCTCCTCAGATG | SEQ ID NO: 6893 |
| rs2523989 | CTGCTTGGTCTTCAGGGAATCAAT | SEQ ID NO: 2162 | CTGCTTGGTCTTCAGGGAATCAAC | SEQ ID NO: 4528 | AGCTTCTTGAGATCGTTCAACT | SEQ ID NO: 6894 |
| rs2071788 | CCAACGGCTCTACCAAGATGATTATA | SEQ ID NO: 2163 | CCAACGGCTCTACCAAGATGATTATG | SEQ ID NO: 4529 | GGCATTTTAATAAGGATTACGT | SEQ ID NO: 6895 |
| rs4713214 | CCCAGGCAGAGTATTCAAAATTTTGT | SEQ ID NO: 2164 | CCCAGGCAGAGTATTCAAAATTTTGA | SEQ ID NO: 4530 | ACTAAAACAGCTCAGTGAGATACC | SEQ ID NO: 6896 |
| rs29273 | CTGAGCAGGGTCTTTGGAATCAG | SEQ ID NO: 2165 | CTGAGCAGGGTCTTTGGAATCAC | SEQ ID NO: 4531 | AATCTCATGTTGGCTTGTTACAT | SEQ ID NO: 6897 |
| rs2517912 | TCAGCCAACACATCTAGCTTAGCATA | SEQ ID NO: 2166 | TCAGCCAACACATCTAGCTTAGCATG | SEQ ID NO: 4532 | TGTCAGTAACTTTGGTTTGGATAC | SEQ ID NO: 6898 |
| rs1265156 | CCCTCCCATCTACCCACCCCA | SEQ ID NO: 2167 | CCCTCCCATCTACCCACCCCC | SEQ ID NO: 4533 | TTACTTGACATGGGAATGTAGAC | SEQ ID NO: 6899 |
| rs4386816 | CATTTGGGTGCGCTAAATCTTTATCT | SEQ ID NO: 2168 | CATTTGGGTGCGCTAAATCTTTATCC | SEQ ID NO: 4534 | AATAAATAAATGGAAACTTCCTTT | SEQ ID NO: 6900 |
| rs2075702 | CAGTGAATGCCCCATGTAAAAACAT | SEQ ID NO: 2169 | CAGTGAATGCCCCATGTAAAAACAC | SEQ ID NO: 4535 | ATGATAAAACAGCAAAGCACTG | SEQ ID NO: 6901 |
| rs1264616 | AGGTCAGAAGGGTGTTTGCTGTTAT | SEQ ID NO: 2170 | AGGTCAGAAGGGTGTTTGCTGTTAC | SEQ ID NO: 4536 | ACTCTTGCTCTCACTTCTACCTG | SEQ ID NO: 6902 |
| rs1419633 | TGATGCTTTGGCTCTTGGAGTATCA | SEQ ID NO: 217T | TGATGCTTTGGCTCTTGGAGTATCG | SEQ ID NO: 4537 | TGTCAACTCAGACAACTGTACTTG | SEQ ID NO: 6903 |
| rs389883 | GCTGCCAAAAAGCTGACTCTTCTTT | SEQ ID NO: 2172 | GCTGCCAAAAAGCTGACTCTTCTTG | SEQ ID NO: 4538 | TAATAAAATGACCCAAGTTTGTATT | SEQ ID NO: 6904 |
| rs2523921 | CCCTGGATTTGGAGATCTCTCCTACA | SEQ ID NO: 2173 | CCCTGGATTTGGAGATCTCTCCTACG | SEQ ID NO: 4539 | CTGCTTAAGCTTCTTCAGCTATC | SEQ ID NO: 6905 |
| rs209474 | CCAAATTAAACCCACCCTTCTAAGAA | SEQ ID NO: 2174 | CCAAATTAAACCCACCCTTCAAAGAG | SEQ ID NO: 4540 | AACCTCAGTCCCCTTATTATTG | SEQ ID NO: 6906 |
| rs2532921 | AACTGGGGCACCTGTTCAATTAGTG | SEQ ID NO: 2175 | AACTGGGGCACCTGTTCAATTAGTC | SEQ ID NO: 4541 | TGATGCCATACTCTGGTAAGAG | SEQ ID NO: 6907 |
| rs2207338 | TGGGTGAGGGGAGATTATATAGTGCA | SEQ ID NO: 2176 | TGGGTGAGGGGAGATTATATAGTGCC | SEQ ID NO: 4542 | ATGTTAAATTTTGCATCATGGTA | SEQ ID NO: 6908 |
| rs2844829 | AAAGTGCACAATGCAGTGACAATTT | SEQ ID NO: 2177 | AAAGTGCACAATGCAGTGACAATTC | SEQ ID NO: 4543 | TATGTTCACAATGTTATGTAACCA | SEQ ID NO: 6909 |
| rs1035798 | TCCACTGGCCTCATTTCCACAT | SEQ ID NO: 2178 | TCCACTGGCCTCATTTCCACAC | SEQ ID NO: 4544 | CAGAGTTTGAGAACCTTCACAAT | SEQ ID NO: 6910 |
| rs2857597 | AACCTCAAAGAATGCAGGCAGGT | SEQ ID NO: 2179 | AACCTCAAAGAATGCAGGCAGGA | SEQ ID NO: 4545 | AAAAACACATGTCCAAACAAG | SEQ ID NO: 6911 |
| rs2747421 | AGCCTGGGCTAGGTGTCTGCCG | SEQ ID NO: 2180 | AGCCTGGGCTAGGTGTCTGCCC | SEQ ID NO: 4546 | CTGTTCTACCCTGCTTCTAGAA | SEQ ID NO: 6912 |
| rs2246818 | GATTGCTTTGATGCTGGGTCAAA | SEQ ID NO: 2181 | GATTGCTTTGATGCTGGGTCAAG | SEQ ID NO: 4547 | CATTGAGACAGACCTCTCCTT | SEQ ID NO: 6913 |
| rs2855812 | GGTGGGCAGGTGACAGGGTA | SEQ ID NO: 2182 | GGTGGGCAGGTGACAGGGTC | SEQ ID NO: 4548 | CAAAGAGTAAGTCCATCTTTTCA | SEQ ID NO: 6914 |
| rs6457259 | GGAATGTTAAATGGTTCAGCCTCTGT | SEQ ID NO: 2183 | GGAATGTTAAATGGTTCAGCCTCTGC | SEQ ID NO: 4549 | GTAAACAGTTTGATGGTTCCTC | SEQ ID NO: 6915 |
| rs2071799 | CAGACACCAAGGGCTCTTATGAATCT | SEQ ID NO: 2184 | CAGACACCAAGGGCTCTTATGAATCC | SEQ ID NO: 4550 | ATCCTGAAAGGAAGGTAAGATT | SEQ ID NO: 6916 |
| rs7754054 | TGAGATTTGTGTGGGGACATAAAACG | SEQ ID NO: 2185 | TGAGATTTGTGTGGGGACATAAAACC | SEQ ID NO: 4551 | AACCATATCAGGCTCTATTCTGT | SEQ ID NO: 6917 |
| rs1049526 | TGATCTGGAGTTACCTGAGGCCAT | SEQ ID NO: 2186 | TGATCTGGAGTTACCTGAGGCCAC | SEQ ID NO: 4552 | GCTGCCCTATTCACTTCTAAG | SEQ ID NO: 6918 |
| rs2284191 | TGAGTGTGGCTCTTCCATAACTGTTA | SEQ ID NO: 2187 | TGAGTGTGGCTCTTCCATAACTGTTG | SEQ ID NO: 4553 | CTAGTTTTCTGGAAGTTAGGGAT | SEQ ID NO: 6919 |
| rs1655944 | ACAGAGCCTTGGGTGCTGCAGA | SEQ ID NO: 2188 | ACAGAGCCTTGGGTGCTGCAGG | SEQ ID NO: 4554 | AAAACAGTTTGAGACAGAGAAAG | SEQ ID NO: 6920 |
| rs7451258 | CCACTGCACCCAAACTTCCTTTG | SEQ ID NO: 2189 | CCACTGCACCCAAACTTCCTTTC | SEQ ID NO: 4555 | TTTTATTTAAAAACAGCAACAACA | SEQ ID NO: 6921 |
| rs2844665 | GGAGGGAAAGCCCTACCACCT | SEQ ID NO: 2190 | GGAGGGAAAGCCCTACCACCC | SEQ ID NO: 4556 | CCTACTGTTTCCTGAAGATTCC | SEQ ID NO: 6922 |
| rs3763364 | TGTTGCATAGGAAGGCTCTGGGT | SEQ ID NO: 2191 | TGTTGCATAGGAAGGCTCTGGGA | SEQ ID NO: 4557 | AGGAAGGATATTTAAAAACCTAATT | SEQ ID NO: 6923 |
| rs3128982 | CTGATCTCTGTTTCCTTTGCCGAT | SEQ ID NO: 2192 | CTGATCTCTGTTTCCTTTGCCGAC | SEQ ID NO: 4558 | AGACTGATTTTCTCTGCTTGG | SEQ ID NO: 6924 |
| rs6457358 | GTTTCACTTCTCCCAACCTGCGA | SEQ ID NO: 2193 | GTTTCACTTCTCCCAACCTGCGT | SEQ ID NO: 4559 | GGGTCCTTCTTCCTGAATACT | SEQ ID NO: 6925 |
| rs7758512 | GTTTGGACAGTGTGTGCCCAGAT | SEQ ID NO: 2194 | GTTTGGACAGTGTGTGCCCAGAG | SEQ ID NO: 4560 | CCACTTGGTGAGAAGAAGATT | SEQ ID NO: 6926 |
| rs3115673 | CAAATGTGTATGGAAGGGTGGACTA | SEQ ID NO: 2195 | CAAATGTGTATGGAAGGGTGGACTC | SEQ ID NO: 4561 | AAATGAACTACAGGAAAATACATG | SEQ ID NO: 6927 |
| rs7029 | CTTCCCCTTCCTGGGATCTGCT | SEQ ID NO: 2196 | CTTCCCCTTCCTGGGATCTGCC | SEQ ID NO: 4562 | AGATGCAGATCCTGAAGTTTTT | SEQ ID NO: 6928 |
| rs1041885 | CACCAAAAATTCCACCCAAGATCT | SEQ ID NO: 2197 | CACCAAAAATTCCACCCAAGATCA | SEQ ID NO: 4563 | CTAAATAGTTTATTGTGTTCAGAAA | SEQ ID NO: 6929 |
| rs259939 | CCCAGCCCAGACATTTCTCCTA | SEQ ID NO: 2198 | CCCAGCCCAGACATTTCTCCTG | SEQ ID NO: 4564 | ATTTCCAACTTGCATATCCAT | SEQ ID NO: 6930 |
| rs3093993 | TGTGACAGTAACAGAAAGTGCCCAGA | SEQ ID NO: 2199 | TGTGACAGTAACAGAAAGTGCCCAGC | SEQ ID NO: 4565 | GGGTCTCTGACACTTAGTAATGT | SEQ ID NO: 6931 |
| rs6457595 | GACCCTCATCAGAAATCCTGACGT | SEQ ID NO: 2200 | GACCCTCATCAGAAATCCTGACGA | SEQ ID NO: 4566 | CTAGCCCTCTTCTGTTTTTTC | SEQ ID NO: 6932 |
| rs3128844 | TGCTTTAGTGAGCTCAGCTTCTCTGG | SEQ ID NO: 2201 | TGCTTTAGTGAGCTCAGCTTCTCTGC | SEQ ID NO: 4567 | AACTTCATCTGTGAAGCTGTCT | SEQ ID NO: 6933 |
| rs3094212 | ATGGAGAAGGGGCAGGAACAAA | SEQ ID NO: 2202 | ATGGAGAAGGGGCAGGAACAAG | SEQ ID NO: 4568 | AGGTCTAAAAGAAAGGACCCT | SEQ ID NO: 6934 |
| rs1264695 | CAGGTTGGAAGACCTCCCCAA | SEQ ID NO: 2203 | CAGGTTGGAAGACCTCCCCAG | SEQ ID NO: 4569 | TTTAAACCTTGCTACAAAGTATTC | SEQ ID NO: 6935 |
| rs2516393 | CGTGGGGTTCATGATTTAGATCACAT | SEQ ID NO: 2204 | CGTGGGGTTCATGATTTAGATCACAG | SEQ ID NO: 4570 | ATTGAAGTCATTTATTATCGGC | SEQ ID NO: 6936 |
| rs209146 | TCGCCTTCATATTCAATCTGTGTTCT | SEQ ID NO: 2205 | TCGCCTTCATATTCAATCTGTGTTCC | SEQ ID NO: 4571 | GCTAGACCAAACTGTTCTCAACT | SEQ ID NO: 6937 |
| rs4713243 | CATCAAAGACTGGAGCGACGTTC | SEQ ID NO: 2206 | CATCAAAGACTGGAGCGACGTTG | SEQ ID NO: 4572 | CTGAAATGATACAAGACCAGC | SEQ ID NO: 6938 |
| rs532385 | TGGGAAATGACCCTTTTTCCTACAA | SEQ ID NO: 2207 | TGGGAAATGACCCTTTTTCCTACAG | SEQ ID NO: 4573 | ATTTCATCAAATAGAGATTCATTTT | SEQ ID NO: 6939 |
| rs1610628 | CCCTAGTGAAAGCTACAGATTTGCCT | SEQ ID NO: 2208 | CCCTAGTGAAAGCTACAGATTTGCCG | SEQ ID NO: 4574 | AGTTCTAGAAACTGCTCCCTTC | SEQ ID NO: 6940 |
| rs8111 | TGGGAATTTAGTCCCCAGGTGGA | SEQ ID NO: 2209 | TGGGAATTTAGTCCCCAGGTGGG | SEQ ID NO: 4575 | CAAGGGAAGTTTTTTCATTTT | SEQ ID NO: 6941 |
| rs2242660 | TGATGAGGGAAGGGCATATGCTTA | SEQ ID NO: 2210 | TGATGAGGGAAGGGCATATGCTTG | SEQ ID NO: 4576 | CACTGCTGAGATAGCTCTGTT | SEQ ID NO: 6942 |
| rs3115592 | TGATGTGGTAGGTGGAAATGGTTAAA | SEQ ID NO: 2211 | TGATGTGGTAGGTGGAAATGGTTAAC | SEQ ID NO: 4577 | TCAATCTAGGAACAAGATTTTTC | SEQ ID NO: 6943 |
| rs3135029 | ACGTGATCTGGCCCTTGCCA | SEQ ID NO: 2212 | ACGTGATCTGGCCCTTGCCC | SEQ ID NO: 4578 | AATTTGTAGCTTATCACAACATCC | SEQ ID NO: 6944 |
| rs3869109 | CCGCTCTGTAAACACCAACTCCCA | SEQ ID NO: 2213 | CCGCTCTGTAAACACCAACTCCCG | SEQ ID NO: 4579 | CAGTTCCAATCTGTAACTTGC | SEQ ID NO: 6945 |
| rs2066951 | GATTTGATGCAGCAGCCTTATGAT | SEQ ID NO: 2214 | GATTTGATGCAGCAGCCTTATGAG | SEQ ID NO: 4580 | CAGAACAGGCTGGGTTACTAT | SEQ ID NO: 6946 |
| rs2282851 | GCACAGTCCCTCCCACGTCT | SEQ ID NO: 2215 | GCACAGTCCCTCCCACGTCC | SEQ ID NO: 4581 | AGCCTAGAAAAGCTTCTGACTC | SEQ ID NO: 6947 |
| rs2517713 | CCAGGGATTTCTGGCTCGCT | SEQ ID NO: 2216 | CCAGGGATTTCTGGCTCGCG | SEQ ID NO: 4582 | CTCATATTCCCTTACAGGTGTG | SEQ ID NO: 6948 |
| rs7770505 | AGTGGAGACGCAATCTCCCTTGA | SEQ ID NO: 2217 | AGTGGAGACGCAATCTCCCTTGG | SEQ ID NO: 4583 | AGACGTTAGTCTCCATCTCTAACG | SEQ ID NO: 6949 |
| rs3117294 | CTCTCCCCAAGCAGAACCCATTT | SEQ ID NO: 2218 | CTCTCCCCAAGCAGAACCCATTG | SEQ ID NO: 4584 | TCCTACACAGGTGTCATCAGA | SEQ ID NO: 6950 |
| rs2857218 | TCCTAACACCGTGGTAGAATCCTTC | SEQ ID NO: 2219 | TCCTAACACCGTGGTAGAATCCTTG | SEQ ID NO: 4585 | TGAAATAGATTAAATTCCCCAAG | SEQ ID NO: 6951 |
| rs2242656 | CCCAAAACAGAACAGGCTGAAATA | SEQ ID NO: 2220 | CCCAAAACAGAACAGGCTGAAATG | SEQ ID NO: 4586 | AGATAACTCTTTCCCTCCCTG | SEQ ID NO: 6952 |
| rs2275454 | GAGGAACCTCCTAGTCTTGGAACCTT | SEQ ID NO: 2221 | GAGGAACCTCCTAGTCTTGGAACCTC | SEQ ID NO: 4587 | AGAAGTTTCCAGAAATGTGTGA | SEQ ID NO: 6953 |
| rs3130050 | CATTGGTCAGGTCTATGCTGTGTTT | SEQ ID NO: 2222 | CATTGGTCAGGTCTATGCTGTGTTC | SEQ ID NO: 4588 | TAAAGGGTAGAAATCAGAACCC | SEQ ID NO: 6954 |
| rs7745413 | CAATCTGTACCTGCAGAAGCCTGATAT | SEQ ID NO: 2223 | CAATCTGTACCTGCAGAAGCCTGATAC | SEQ ID NO: 4589 | GCTAAAGAATGAATGGAATTACTC | SEQ ID NO: 6955 |
| rs2894066 | CCCACTCCCACCTTGTTCTACCT | SEQ ID NO: 2224 | CCCACTCCCACCTTGTTCTACCC | SEQ ID NO: 4590 | ACTCATCTATTTTGTTCTACCTTTT | SEQ ID NO: 6956 |
| rs3213469 | CCACAGGAGGCATCCTGAAGGG | SEQ ID NO: 2225 | CCACAGGAGGCATCCTGAAGGC | SEQ ID NO: 4591 | ACAGGGACTCTGTCTTCACTT | SEQ ID NO: 6957 |
| rs406936 | GTGGCCACAGGAACATGGACAA | SEQ ID NO: 2226 | GTGGCCACAGGAACATGGACAG | SEQ ID NO: 4592 | TGTTGAAACACTAGGTGGAGAA | SEQ ID NO: 6958 |
| rs3916765 | GGGACCACACTCTTGCCTTCCTAA | SEQ ID NO: 2227 | GGGACCACACTCTTGCCTTCCTAG | SEQ ID NO: 4593 | ACTCACGTACCATTTTCAAGC | SEQ ID NO: 6959 |
| rs3873257 | GTGATCAGAATGAGTCAGGGTGGT | SEQ ID NO: 2228 | GTGATCAGAATGAGTCAGGGTGGA | SEQ ID NO: 4594 | CAGGTAACTGGAATGAGTCAG | SEQ ID NO: 6960 |
| rs453658 | CAGCAGAAAGATTTCTGTCCATCACA | SEQ ID NO: 2229 | CAGCAGAAAGATTTCTGTCCATCACG | SEQ ID NO: 4595 | AAACTTTCATCCATTACATAACC | SEQ ID NO: 6961 |
| rs660594 | CCAGTGACACCAAGGCACTCTACAA | SEQ ID NO: 2230 | CCAGTGACACCAAGGCACTCTACAG | SEQ ID NO: 4596 | GCAAGTATTGCTTTGTTTTCC | SEQ ID NO: 6962 |
| rs1008403 | AAAACAGAGAGAGAGGTGGGAGGAA | SEQ ID NO: 2231 | AAAACAGAGAGAGAGGTGGGAGGAG | SEQ ID NO: 4597 | GGAGAAGAGTATTCTAGGAGGAA | SEQ ID NO: 6963 |
| rs763009 | TTGAGAGTTCTCTGGGAAGCATAGAA | SEQ ID NO: 2232 | TTGAGAGTTCTCTGGGAAGCATAGAG | SEQ ID NO: 4598 | ATTTATTCTGCTCTTGATGAGG | SEQ ID NO: 6964 |
| rs3130649 | CAGAGGACATCACTCCAGGACTTGA | SEQ ID NO: 2233 | CAGAGGACATCACTCCAGGACTTGT | SEQ ID NO: 4599 | TGTAGCCTCTGAAAGTAGAGGC | SEQ ID NO: 6965 |
| rs7752270 | GAATTTAGTGAAATCACCAGAGCCTGA | SEQ ID NO: 2234 | GAATTTAGTGAAATCACCAGAGCCTGG | SEQ ID NO: 4600 | AAATAGGAAAATGAGTTCAAAGG | SEQ ID NO: 6966 |
| rs659445 | TTGTGGCATGGCCTAGAAAACAA | SEQ ID NO: 2235 | TTGTGGCATGGCCTAGATAACAG | SEQ ID NO: 4601 | CAAGCAAAAGGCAAGATAAGA | SEQ ID NO: 6967 |
| rs7753474 | GCCAATGTGACAAGCTCTTTGTTTT | SEQ ID NO: 2236 | GCCAATGTGACAAGCTCTTTGTTTC | SEQ ID NO: 4602 | GTTGGAGTGTTTGCACAATTTA | SEQ ID NO: 6968 |
| rs3130762 | CCCTGTGGTTCAGCTTCTTGAACC | SEQ ID NO: 2237 | CCCTGTGGTTCAGCTTCTTGAACG | SEQ ID NO: 4603 | TGTGATAGACACACCTGCAAC | SEQ ID NO: 6969 |
| rs3094127 | CTGAGAAAAGCAGTGATATGCCCTA | SEQ ID NO: 2238 | CTGAGAAAAGCAGTGATATGCCCTG | SEQ ID NO: 4604 | AATTAATTAACTGTCAATAGGCTG | SEQ ID NO: 6970 |
| rs7754598 | TTTGCATTATGAAAATCCAGGTCAA | SEQ ID NO: 2239 | TTTGCATTATGAAAATCCAGGTCAG | SEQ ID NO: 4605 | TCTTCTTTCTTCTCCCAATTGT | SEQ ID NO: 6971 |
| rs7774255 | TGTCAAGTAACTCAATTCCTCAGCAA | SEQ ID NO: 2240 | TGTCAAGTAACTCAATTCCTCAGCAT | SEQ ID NO: 4606 | GAGGTACTGAAATGAGATGAATTT | SEQ ID NO: 6972 |
| rs465506 | AGGCTTCAATGGAGCGCAAAAA | SEQ ID NO: 2241 | AGGCTTCAATGGAGCGCAAAAG | SEQ ID NO: 4607 | TCCTAAATTTGGTGAGAAATG | SEQ ID NO: 6973 |
| rs2853961 | ATTCGGGGTCCTTGACTTCCCA | SEQ ID NO: 2242 | ATTCGGGGTCCTTGACTTCCCG | SEQ ID NO: 4608 | AACAACAGTAAGAGTAAGCAAGTAA | SEQ ID NO: 6974 |
| rs3815101 | CCTCGCCTTTTACCCTTCCCTA | SEQ ID NO: 2243 | CCTCGCCTTTTACCCTTCCCTG | SEQ ID NO: 4609 | CTCCTTTCCTCTCCTCAGAGAT | SEQ ID NO: 6975 |
| rs2517892 | TGAAAATGGACAGACTTAAAACCCAAT | SEQ ID NO: 2244 | TGAAAATGGACAGACTTAAACCCAAC | SEQ ID NO: 4610 | AGGTCAACAATGACATTAAACG | SEQ ID NO: 6976 |
| rs2187689 | GGAGTACAGTGACCTGAACATGGCTT | SEQ ID NO: 2245 | GGAGTACAGTGACCTGAACATGGCTC | SEQ ID NO: 4611 | CTGTATCCCTAACCTGCTGTG | SEQ ID NO: 6977 |
| rs6901092 | AGAGGCTCTAGTAGCAGCTTTGCAA | SEQ ID NO: 2246 | AGAGGCTCTAGTAGCAGCTTTGCAG | SEQ ID NO: 4612 | CTTGCAAAGTCCAAGATTTCT | SEQ ID NO: 6978 |
| rs2857605 | CAGTTGGCATGGTAGAACTTCTAGGAT | SEQ ID NO: 2247 | CAGTTGGCATGGTAGAACTTCTAGGAC | SEQ ID NO: 4613 | CTGAAATAGTTTGTGGAGACACA | SEQ ID NO: 6979 |
| rs2516676 | CGTGGGGTAGGAGAAACAGGTATAACC | SEQ ID NO: 2248 | CGTGGGGTAGGAGAAACAGGTATAACG | SEQ ID NO: 4614 | TTGACTAGAGAATGGATACTTGAG | SEQ ID NO: 6980 |
| rs6922756 | GCTCAGGAGTGAAGCTACAGAGCTTT | SEQ ID NO: 2249 | GCTCAGGAGTGAAGCTACAGAGCTTG | SEQ ID NO: 4615 | CGGTCAGTGTTATAGCTCTTAAG | SEQ ID NO: 6981 |
| rs2074475 | AAAGGAAAAGAGGCTAGAAGGGAAGA | SEQ ID NO: 2250 | AAAGGAAAAGAGGCTAGAAGGGAAGG | SEQ ID NO: 4616 | AAAAAAAGGATCAACGAA4ATA | SEQ ID ND: 6982 |
| rs1264432 | TTTTAACTTCTGTGATCCCTGTAGCCA | SEQ ID NO: 2251 | TTTTAACTTCTGTGATCCCTGTAGCCG | SEQ ID NO: 4617 | TCATATGTGCTGTTAATGGAATT | SEQ ID NO: 6983 |
| rs2267633 | GGGGAGCCACATGTGAGAAAGTATA | SEQ ID NO: 2252 | GGGGAGCCACATGTGAGAAAGTATG | SEQ ID NO: 4618 | AAGGGCAAGTAAGATGGAAAG | SEQ ID NO: 6984 |
| rs2517819 | GAGGGTCCGCCGTGCTCTT | SEQ ID NO: 2253 | GAGGGTCCGCCGTGCTCTC | SEQ ID NO: 4619 | GACACAATTAAGGGATGAAATCT | SEQ ID NO: 6985 |
| rs3130637 | CACTTTCTATCTCATTTCATGAGGCCA | SEQ ID NO: 2254 | CACTTTCTATCTCATTTCATGAGGCCG | SEQ ID NO: 4620 | GATTCCATGAGGGAATACTTTC | SEQ ID NO: 6986 |
| rs3116837 | TCTGAGGCCTTATTGTAACAAAATGGT | SEQ ID NO: 2255 | TCTGAGGCCTTATTGTAACAAAATGGC | SEQ ID NO: 4621 | ATCTTTTTATCAGACCCAATTATT | SEQ ID NO: 6987 |
| rs3793125 | CTCACATGCAAGGGGAGGGA | SEQ ID NO: 2256 | CTCACATGCAAGGGGAGGGG | SEQ ID NO: 4622 | CTGAGTCAGGATAGAGATGCCT | SEQ ID NO: 6988 |
| rs757257 | TCTCATGTGGTGGTTGAGAAAACTGA | SEQ ID NO: 2257 | TCTCATGTGGTGGTTGAGAAAACTGG | SEQ ID NO: 4623 | ACACAGTTAAATGAACTTGTCTAAC | SEQ ID NO: 6989 |
| rs3117038 | TACCTGGAGGCTGCCATGACACT | SEQ ID NO: 2258 | TACCTGGAGGCTGCCATGACACC | SEQ ID NO: 4624 | GTAAACGTGGTGACAAGGAAA | SEQ ID NO: 6990 |
| rs2523589 | TTCTAGAATGTAATGGACGTGCAGTCT | SEQ ID NO: 2259 | TTCTAGAATGTAATGGACGTGCAGTCG | SEQ ID NO: 4625 | ATGTTATGTAGTCCTTCGTTTGG | SEQ ID NO: 6991 |
| rs2747479 | GTTTTTCAAATCTTGCTAGTGAGCCT | SEQ ID NO: 2260 | GTTTTTCAAATCTTGCTAGTGAGCCG | SEQ ID NO: 4626 | TTGCTAATTAAAAGTGATCTTCTG | SEQ ID NO: 6992 |
| rs3130244 | TCTCTGAGGAGAGCTTGGGAAGGA | SEQ ID NO: 2261 | TCTCTGAGGAGAGCTTGGGAAGGC | SEQ ID NO: 4627 | TGTTCATGGTCTCAGGCTCTAT | SEQ ID NO: 6993 |
| rs7357041 | CCAGACGTTTATCTTGCTGAGGCTT | SEQ ID NO: 2262 | CCAGACGTTTATCTTGCTGAGGCTC | SEQ ID NO: 4628 | GTGCTCTCATCCAGAGAATG | SEQ ID NO: 6994 |
| rs4713504 | TTAAGAGGGAGCCAAAGTGAGGTAC | SEQ ID NO: 2263 | TTAAGAGGGAGCCAAAGTGAGGTAG | SEQ ID NO: 4629 | CAAGAGAGGGATCCTTCTTTC | SEQ ID NO: 6995 |
| rs2187580 | CCCAAATGCCTATCAATGAAAGACTAGA | SEQ ID NO: 2264 | CCCAAATGCCTATCAATGAAAGACTAGG | SEQ ID NO: 4630 | AAAGAAAATGTAGTATGCAGCC | SEQ ID NO: 6996 |
| rs3132945 | GGACAAGAAATGCCACATTTTAAAAAT | SEQ ID NO: 2265 | GGACAAGAAATGCCACATTTTAAAAAC | SEQ ID NO: 4631 | AAAATTATCAAACTGGATTAAATGG | SEQ ID NO: 6997 |
| rs3998751 | GAAACTGTGGGAGATGATGAATATTTGT | SEQ ID NO: 2266 | GAAACTGTGGGAGATGATGAATATTTGC | SEQ ID NO: 4632 | GTTTTGAGCTGCTAAGTTTTACA | SEQ ID NO: 6998 |
| rs2860580 | AAGCAAACCCGTCCTTCTTCAT | SEQ ID NO: 2267 | AAGCAAACCCGTCCTTCTTCAC | SEQ ID NO: 4633 | TGGTGACAGGAGAGAGAAATG | SEQ ID NO: 6999 |
| rs7757916 | CTTGGGGGAAGCATCAGGAGATA | SEQ ID NO: 2268 | CTTGGGGGAAGCATCAGGAGATG | SEQ ID NO: 4634 | ACTTGAAAAAACTCTTTTTACTAAGA | SEQ ID NO: 7000 |
| rs3130014 | TGAATTTGTCTAGGTGTTAGATTGGCT | SEQ ID NO: 2269 | TGAATTTGTCTAGGTGTTAGATTGGCC | SEQ ID NO: 4635 | CAGTTCTCTACCACTTCCTGTTA | SEQ ID NO: 7001 |
| rs3830076 | ACTGGGTACTACATCTCCCAGGCA | SEQ ID NO: 2270 | ACTGGGTACTACATCTCCCAGGCG | SEQ ID NO: 4636 | TTTATCACTGGAGCCTGTTTG | SEQ ID NO: 7002 |
| rs2158280 | AACAAATTTGATAAAATCCAACACCCA | SEQ ID NO: 2271 | AACAAATTTGATAAAATCCAACACCCG | SEQ ID NO: 4637 | TTATGATAAAAACTCTCAGCAAGC | SEQ ID NO: 7003 |
| rs1233391 | CCGCCTGGACACCGAGCTT | SEQ ID NO: 2272 | CCGCCTGGACACCGAGCTC | SEQ ID NO: 4638 | TGAAGTTAGCTGGAAGTACCAG | SEQ ID NO: 7004 |
| rs2844477 | AATCCATGAGGTTCACATGGACTTAT | SEQ ID NO: 2273 | AATCCATGAGGTTCACATGGACTTAC | SEQ ID NO: 4639 | GTTACACAAATTAGAAACAAATGTT | SEQ ID NO: 7005 |
| rs7453097 | GAGCGAAACCGCCTCTCAAACT | SEQ ID NO: 2274 | GAGCGAAACCGCCTCTCAAACA | SEQ ID NO: 4640 | ACAAAAAAAAACTTTGGGAGG | SEQ ID NO: 7006 |
| rs3130816 | CTCTCTCTCCCTCGTGAGAAAGAA | SEQ ID NO: 2275 | CTCTCTCTCCCTCGTGAGAAAGAG | SEQ ID NO: 4641 | TTTTTGAAAGGGAAACAACTG | SEQ ID NO: 7007 |
| rs4151648 | CTCAGCCAGGGCTGCATGCA | SEQ ID NO: 2276 | CTCAGCCAGGGCTGCATGCG | SEQ ID NO: 4642 | AAGAGATTGATGTGAAAGTCTCG | SEQ ID NO: 7008 |
| rs3131090 | TCTTTGAGACTCAAATTTGCCATTTA | SEQ ID NO: 2277 | TCTTTGAGACTCAAATTTGCCATTTG | SEQ ID NO: 4643 | TAGTGCGTGCATTTAGACAAG | SEQ ID NO: 7009 |
| rs1063318 | ACGTCGCTGTCGAAGCGCA | SEQ ID NO: 2278 | ACGTCGCTGTCGAAGCGCG | SEQ ID NO: 4644 | GTACTCCTCTCGGTTATAGATGTA | SEQ ID NO: 7010 |
| rs3130660 | CAAAACATTTTCATCAACTGATCAACAA | SEQ ID NO: 2279 | CAAAACATTTTCATCAACTGATCAACAT | SEQ ID NO: 4645 | GAGCCTTGCTTTATATGTGTTT | SEQ ID NO: 7011 |
| rs1264549 | GTCAAAGATCAAGGCCTGGGAAA | SEQ ID NO: 2280 | GTCAAAGATCAAGGCCTGGGAAG | SEQ ID NO: 4646 | GTCAGTGTATGGTGAGGGCT | SEQ ID NO: 7012 |
| rs2299851 | CATCTTATATAAGAGCCAACATGTGCAT | SEQ ID NO: 2281 | CATCTTATATAAGAGCCAACATGTGCAC | SEQ ID NO: 4647 | CACTAGGTCCTGAAGATTTGTATC | SEQ ID NO: 7013 |
| rs2844682 | TCATGACCATCTCTATGGCCAATT | SEQ ID NO: 2282 | TCATGACCATCTCTATGGCCAATC | SEQ ID NO: 4648 | CTTGGAGACCTCAGAGAAGC | SEQ ID NO: 7014 |
| rs2051625 | TTTCCTAATGGCCCTGATCCTGTT | SEQ ID NO: 2283 | TTTCCTAATGGCCCTGATCCTGTC | SEQ ID NO: 4649 | GCTTCTTCTCCTTCCGAGAT | SEQ ID NO: 7015 |
| rs2178899 | TGTCCCTTAATCTTGTGTGAACTTTTTT | SEQ ID NO: 2284 | TGTCCCTTAATCTTGTGTGAACTTTTTA | SEQ ID NO: 4650 | AATTAATACTTCCTTGGAACACTG | SEQ ID NO: 7016 |
| rs3130389 | GGAGAGTGATCTATTGGTTAGGAGCACA | SEQ ID NO: 2285 | GGAGAGTGATCTATTGGTTAGGAGCACG | SEQ ID NO: 4651 | AATAAAAGATATTGGAAATATTCGA | SEQ ID NO: 7017 |
| rs3749981 | TTGGGGTTTTGAGCCGCAGTA | SEQ ID NO: 2286 | TTGGGGTTTTGAGCCGCAGTG | SEQ ID NO: 4652 | CTGCCTCTGTTCAAGTATCCA | SEQ ID NO: 7018 |
| rs1233386 | TTATTTTAAGGAAGTGAAACCCCTTCA | SEQ ID NO: 2287 | TTATTTTAAGGAAGTGAAACCCCTTCG | SEQ ID NO: 4653 | CTGGGGGATCATTTAGATATAGT | SEQ ID NO: 7019 |
| rs2142234 | CCCGGAGCAGCCCTGATT | SEQ ID NO: 2288 | CCCGGAGCAGCCCTGATC | SEQ ID NO: 4654 | GTGAGAGGAGAGAAGACCAAG | SEQ ID NO: 7020 |
| rs2747419 | TGACAGTGTTGAAAGATTCCAGTTAAA | SEQ ID NO: 2289 | TGACAGTGTTGAAAGATTCCAGTTAAC | SEQ ID NO: 4655 | TTTTATTTGCTTCTAATTTGAAAA | SEQ ID NO: 7021 |
| rs356971 | GGAAGTTGTGGTCTGCTATTGGTTATAAT | SEQ ID NO: 2290 | GGAAGTTGTGGTCTGCTATTGGTTATAAG | SEQ ID NO: 4656 | AGTCACAGATCATTATGCTTTCT | SEQ ID NO: 7022 |
| rs1079541 | GCCAGTGATTATTCCACTCTCTACTGA | SEQ ID NO: 2291 | GCCAGTGATTATTCCACTCTCTACTGC | SEQ ID NO: 4657 | TTTAATTGGAGATCTACATTAAGCT | SEQ ID NO: 7023 |
| rs2273192 | GCCTGCTGCCCAGCCACT | SEQ ID NO: 2292 | GCCTGCTGCCCAGCCACC | SEQ ID NO: 4658 | CACCTGCTAGTCTTCGATGTAG | SEQ ID NO: 7024 |
| rs1015466 | AGAAGAGAACTAAATGGACAGAGGGAT | SEQ ID NO: 2293 | AGAAGAGAACTAAATGGACAGAGGGAA | SEQ ID NO: 4659 | CTCAAATTTTTTTGAGATTAAAATT | SEQ ID NO: 7025 |
| rs4947324 | GAAAAGACTGTGGTCCGGTGGA | SEQ ID NO: 2294 | GAAAAGACTGTGGTCCGGTGGG | SEQ ID NO: 4660 | CCAGGTTTCAGGGCTTATTT | SEQ ID NO: 7026 |
| rs2071295 | TTAGTTCTCCCGGGCCAGCCA | SEQ ID NO: 2295 | TTAGTTCTCCCGGGCCAGCCG | SEQ ID NO: 4661 | TGCAGAATAAGAAAGGGAAC | SEQ ID NO: 7027 |
| rs3130010 | AGGCCATCGCCAATCATTGTATAT | SEQ ID NO: 2296 | AGGCCATCGCCAATCATTGTATAC | SEQ ID NO: 4662 | ACAAATGCTCCTTCTGGGATTA | SEQ ID NO: 7028 |
| rs2517424 | GAGTTGTCTGCATTTCCCCCAA | SEQ ID NO: 2297 | GAGTTGTCTGCATTTCCCCCAT | SEQ ID NO: 4663 | AATGACGAGAAGCTTCCTG | SEQ ID NO: 7029 |
| rs3093986 | GCCAACTTTCAATGTTAATTAGTTGTGGT | SEQ ID NO: 2298 | GCCAACTTTCAATGTTAATTAGTTGTGGA | SEQ ID NO: 4664 | TGTTTAACCATATACTGCATAGTTT | SEQ ID NO: 7030 |
| rs2006752 | AGTCATTTTTCCACATCATTTGACTAT | SEQ ID NO: 2299 | AGTCATTTTTCCACATCATTTGACTAC | SEQ ID NO: 4665 | ACTGTCAGTTTAAATTGCTCTAGG | SEQ ID NO: 7031 |
| rs2530699 | TCAACATGCTCCCTTAAGTAATTCTGAT | SEQ ID NO: 2300 | TCAACATGCTGCCTTAAGTAATTCTGAA | SEQ ID NO: 4666 | TAAGTGTGTTCTGAATATTATTCTGA | SEQ ID NO: 7032 |
| rs707917 | CAGCCCTCAGGTGAGTGGGAC | SEQ ID NO: 2301 | CAGCCCTCAGGTGAGTGGGAG | SEQ ID NO: 4667 | CCTTCAAGAAATCCACAGC | SEQ ID NO: 7033 |
| rs6531 | GAACATCAATGGATCGGTGTGAA | SEQ ID NO: 2302 | GAACATCAATGGATCGGTGTGAG | SEQ ID NO: 4668 | AGGAGGCAATGAGGAGTTC | SEQ ID NO: 7034 |
| rs2206042 | CAGCTCACAAAACAGAATAGTTCAGTA | SEQ ID NO: 2303 | CAGCTCACAAAACAGAATAGTTCAGTC | SEQ ID NO: 4669 | CACATTTGTTGCTCTTTTTATTAT | SEQ ID NO: 7035 |
| rs6457452 | AGCTGCTGCGAGGGTCCGT | SEQ ID NO: 2304 | AGCTGCTGCGAGGGTCCGC | SEQ ID NO: 4670 | TCGTCTTTCGAGAGTGACTC | SEQ ID NO: 7036 |
| rs1362064 | CACCAATATCATCCCCCCAAAATT | SEQ ID NO: 2305 | CACCAATATCATCCCCCCAAAATG | SEQ ID NO: 4671 | ATTATAGGTATGAGCCATTCCAC | SEQ ID NO: 7037 |
| rs4424066 | AAAGTGGACACACCATGACTGGAA | SEQ ID NO: 2306 | AAAGTGGACACACCATGACTGGAG | SEQ ID NO: 4672 | AAGAACAAGCAATAACGTAACAG | SEQ ID NO: 7038 |
| rs3094110 | CCCTCCTACCCCAACTGCTAGAAA | SEQ ID NO: 2307 | CCCTCCTACCCCAACTGCTAGAAG | SEQ ID NO: 4673 | GAGGGAGAGATACAGATGAAATAA | SEQ ID NO: 7039 |
| rs2242668 | CCTGTTCCAACAAGTCTGTTGGGA | SEQ ID NO: 2308 | CCTGTTCCAACAAGTCTGTTGGGG | SEQ ID NO: 4674 | GGAACCTCTTTAATTTGAGAAAT | SEQ ID NO: 7040 |
| rs6910451 | CGTAGGCACCCAAGGGAATCTA | SEQ ID NO: 2309 | CGTAGGCACCCAAGGGAATCTG | SEQ ID NO: 4675 | TGTTCTGTGGTTTGCGAAAT | SEQ ID NO: 7041 |
| rs3823355 | CCCACCGGGGTCAAGGAACT | SEQ ID NO: 2310 | CCCACCGGGGTCAAGGAACC | SEQ ID NO: 4676 | AAAGAGGATGAGGGTCAAGG | SEQ ID NO: 7042 |
| rs3130667 | CCAGCTGCTTGCCCAGGTCA | SEQ ID NO: 2311 | CCAGCTGCTTGCCCAGGTCG | SEQ ID NO: 4677 | TGGGGACCATTATGAGTCAG | SEQ ID NO: 7043 |
| rs2071293 | CTTGCTGGAGATGCTGCAGAGA | SEQ ID NO: 2312 | CTTGCTGGAGATGCTGCAGAGG | SEQ ID NO: 4678 | CTCAGTGCCCAGTGTCAAG | SEQ ID NO: 7044 |
| rs1063478 | CCCAAATCCTTCCACAGGGAT | SEQ ID NO: 2313 | CCCAAATCCTTCCACAGGGAC | SEQ ID NO: 4679 | GAATGATGCTGCCAGTTCAC | SEQ ID NO: 7045 |
| rs2242654 | TGTTATCACAACTGTGCAGCCTCCT | SEQ ID NO: 2314 | TGTTATCACAACTGTGCAGCCTCCC | SEQ ID NO: 4680 | TTCTTCAGTTCTTCCTGCATAC | SEQ ID NO: 7046 |
| rs2394994 | CAAATGGCGGCTCCATCTTCT | SEQ ID NO: 2315 | CAAATGGCGGCTCCATCTTCA | SEQ ID NO: 4681 | TTTTCCTTTCCAACCACGT | SEQ ID NO: 7047 |
| rs3130805 | TGCATGATTATATGTGTTTGCAAAAAA | SEQ ID NO: 2316 | TGCATGATTATATGTGTTTGCAAAAAG | SEQ ID NO: 4682 | CAAGTAATTATATGCTAAAAAAGAGA | SEQ ID NO: 7048 |
| rs2269429 | GCCCGGTGTCCACCGTGA | SEQ ID NO: 2317 | GCCCGGTGTCCACCGTGG | SEQ ID NO: 4683 | CGTGACTGGTGAGTAGTGCTT | SEQ ID NO: 7049 |
| rs3132587 | ACCAGTAACGGTCGCGACCCA | SEQ ID NO: 2318 | ACCAGTAACGGTCGCGACCCG | SEQ ID NO: 4684 | GAAAAAGAGCTTATTTGCTGATT | SEQ ID NO: 7050 |
| rs2300825 | TTTCTAGACAGTTGCCTTATCAAATACCA | SEQ ID NO: 2319 | TTTCTAGACAGTTGCCTTATCAAATACCG | SEQ ID NO: 4685 | CTATGTTACGTCATTTAATCTCACA | SEQ ID NO: 7051 |
| rs7744304 | CAGCCAACAAACATAGGATAAAATTCT | SEQ ID NO: 2320 | CAGCCAACAAACATAGGAAAAAATTCG | SEQ ID NO: 4686 | AACAGCACTAATTATTAGAGATATGC | SEQ ID NO: 7052 |
| rs1233388 | AAATTAATTAGGATGACACATGATGGATA | SEQ ID NO: 2321 | AAATTAATTAGGATGACACATGATGGATG | SEQ ID NO: 4687 | ATAAGAAACAATTGGCTATTGTT | SEQ ID NO: 7053 |
| rs4711279 | ACTTTCTTCCGCCTGGCCAGAG | SEQ ID NO: 2322 | ACTTTCTTCCGCCTGGCCAGAC | SEQ ID NO: 4688 | TTTTGTTTTTCAATTTTTTCAGAAA | SEQ ID NO: 7054 |
| rs6915800 | CCCATCCTTACCGGAGTGTGCA | SEQ ID NO: 2323 | CCCATCCTTACCGGAGTGTGCG | SEQ ID NO: 4689 | AGAGATTAAGGGGAAGGTAGG | SEQ ID NO: 7055 |
| rs11908 | GGGGCAGATGGGATGCTCAA | SEQ ID NO: 2324 | GGGGCAGATGGGATGCTCAG | SEQ ID NO: 4690 | GTGCAAAACAATAAGTCTCCTTA | SEQ ID NO: 7056 |
| rs1059288 | GGCTCCTCGAGGACAGGGACT | SEQ ID NO: 2325 | GGCTCCTCGAGGACAGGGACC | SEQ ID NO: 4691 | CTGTCTGTCCACTGCTGTAAC | SEQ ID NO: 7057 |
| rs3025657 | AAGCACTTGCTTATATGTTATTCTCTCCA | SEQ ID NO: 2326 | AAGCACTTGCTTATATGTTATTCTCTCCC | SEQ ID NO: 4692 | GAATTGAATTTACTCCTCAAAGTAA | SEQ ID NO: 7058 |
| rs213209 | TCATTCAGTGTCATAAGTCAGTGCAT | SEQ ID NO: 2327 | TCATTCAGTGTCATAAGTCAGTGCAC | SEQ ID NO: 4693 | TTTATTAAAAGCAGAGCTTCATG | SEQ ID NO: 7059 |
| rs7766900 | TCTTTGCTTTCACTATCCTATGGCCT | SEQ ID NO: 2328 | TCTTTGCTTTCACTATCCTATGGCCC | SEQ ID NO: 4694 | TTTATAGTTGGCTGATAAATATGTG | SEQ ID NO: 7060 |
| rs3130268 | TCACAGCTTTGCCTCTTTTTTTTTTTT | SEQ ID NO: 2329 | TCACAGCTTTGCCTCTTTTTTTTTTTG | SEQ ID NO: 4695 | TGTATTTATTCCAGACCTTTCTG | SEQ ID NO: 7061 |
| rs3749952 | CCCTAGCAGGGAGCTGAGCTA | SEQ ID NO: 2330 | CCCTAGCAGGGAGCTGAGCAC | SEQ ID NO: 4696 | ATCCCAACAAACTGGGGTT | SEQ ID NO: 7062 |
| rs6456951 | TCTTAACTGAAATCCAAGTTCCAATAAA | SEQ ID NO: 2331 | TCTTAACTGAAATCCAAGTTCCAATAAT | SEQ ID NO: 4697 | ATCAAGAGTATAACATTCCTCTTT | SEQ ID NO: 7063 |
| rs2523974 | AGGCAACAACCTTGATCTTGGACTT | SEQ ID NO: 2332 | AGGCAACAACCTTGATCTTGGACTG | SEQ ID NO: 4698 | GAAAATAAATTGCTGTTGCTAGA | SEQ ID NO: 7064 |
| rs6904236 | GGAACCGCACCAACTGGCA | SEQ ID NO: 2333 | GGAACCGCACCAACTGGCG | SEQ ID NO: 4699 | AGCTGCTGAAATTCCTGAATA | SEQ ID NO: 7065 |
| rs4713518 | TGATTTCTTAAGGTAAGTTTCACATTCCTA | SEQ ID NO: 2334 | TGATTTCTTAAGGTAAGTTTCACATTCCTG | SEQ ID NO: 4700 | TTTTATATTGCCACTATCTTAGAAG | SEQ ID NO: 7066 |
| rs2267646 | CAGAGGCCTTTTCCCCTCCCA | SEQ ID NO: 2335 | CAGAGGCCTTTTCCCCTCCCC | SEQ ID NO: 4701 | CTCCCAAATCTGCTTTTCTC | SEQ ID NO: 7067 |
| rs502626 | AAAATATTGATAATCTCAGCTCAAAATAAT | SEQ ID NO: 2336 | AAAATATTGAAAATCTCAGCTCAAAATAAC | SEQ ID NO: 4702 | CACAGAAGATATTGAAAATATGAAA | SEQ ID NO: 7068 |
| rs6468 | CCCCGGGCTTCTTGCACT | SEQ ID NO: 2337 | CCCCGGGCTTCTTGCACC | SEQ ID NO: 4703 | ATCTATCTGCTTGGCCTGACT | SEQ ID NO: 7069 |
| rs3129110 | TTGCTGTGCAGCATATTCTTATATACATA | SEQ ID NO: 2338 | TTGCTGTGCAGCATATTCTTATATACATG | SEQ ID NO: 4704 | TCAATTATCTTTTCTTATTTTAACTC | SEQ ID NO: 7070 |
| rs6456947 | CACAAAAGAAATAAATAAAAGGCATCCAA | SEQ ID NO: 2339 | CACAAAAGAAATAAATAAAAGGCATCCAG | SEQ ID NO: 4705 | CTAAAAAGGAAGAAGTAAAATTTTC | SEQ ID NO: 7071 |
| rs3129154 | CATGTTTTTCAAGAGTCAAGATTACAATAT | SEQ ID NO: 2340 | CATCTTTTTCTAGAGTCAAGATTACAATAC | SEQ ID NO: 4706 | TGAGTTATTAAAAGTTTTTCAAAACA | SEQ ID NO: 7072 |
| rs1009382 | AGAGGCTCCTCGGGGGGCT | SEQ ID NO: 2341 | AGAGGCTCCTCGGGGGGCC | SEQ ID NO: 4707 | CTCTGTGCCTGGTTCTGTAG | SEQ ID NO: 7073 |
| rs1043618 | CCCTGCTCTCTGTCGGCTCC | SEQ ID NO: 2342 | CCCTGCTCTCTGTCGGCTCG | SEQ ID NO: 4708 | CTCTGAGATTGGGGGCTG | SEQ ID NO: 7074 |
| rs7754402 | AGAAATTTGACAAAAGATTACAGGAACT | SEQ ID NO: 2343 | AGAAATTTGACAAAAGATTACAGGAACC | SEQ ID NO: 4709 | AAAAACATAAGTGACTTGATGGA | SEQ ID NO: 7075 |
| rs2269706 | GAGGCCCAAGGCAACAGTTCT | SEQ ID NO: 2344 | GAGGCCCAAGGCAACAGTTCC | SEQ ID NO: 4710 | AGAGGCCTGTGGAAGATG | SEQ ID NO: 7076 |
| rs4713505 | GCATGGTGCTATAAAATTGATGATGAA | SEQ ID NO: 2345 | GCATGGTGCTATATTTAAGATGATGATG | SEQ ID NO: 4711 | TTATTATTATTTATAGAGTCTTGCTCT | SEQ ID NO: 7077 |
| rs205000 | CCAAGAACACAGGCCTCCCCG | SEQ ID NO: 2346 | CCAAGAACACAGGCCTCCCCC | SEQ ID NO: 4712 | TTCTTCAGCTCTCTGAAGTTTC | SEQ ID NO: 7078 |
| rs2071278 | GGAAGGGACAACATGTAAGCTCAGA | SEQ ID NO: 2347 | GGAAGGGACAACATGTAAGCTCAGG | SEQ ID NO: 4713 | AGAATCTAACCTGAGGTGTT | SEQ ID NO: 7079 |
| rs477005 | TCCCAATTCCCTAGTTGTTTTTTCTA | SEQ ID NO: 2348 | TCCCAATTCCCTAGTTGTTTTTTCTG | SEQ ID NO: 4714 | GGTACCATAAAGACTTCTAGCAGA | SEQ ID NO: 7080 |
| rs535586 | CCGGGCAGAACCTAACTCCTCT | SEQ ID NO: 2349 | CCGGGCAGAACCTAACTCCTCC | SEQ ID NO: 4715 | ACTAGAAAAAGATCAGAAAAATTGA | SEQ ID NO: 7081 |
| rs1052248 | CATCTCGAGCCTCCGTTCAAAA | SEQ ID NO: 2350 | CATCTCGAGCCTCCGTTCAAAT | SEQ ID NO: 4716 | GATCATCATCAAAACTTATGTGG | SEQ ID NO: 7082 |
| rs621701 | GGGGACAAGGCAACATACCTCAA | SEQ ID NO: 2351 | GGGGACAAGGCAACATACCTCAG | SEQ ID NO: 4717 | TTCCTTATCCATAAAATAGGGG | SEQ ID NO: 7063 |
| rs2072679 | TCCCTCCCTCCACATATACATGCA | SEQ ID NO: 2352 | TCCCTCCCTCCACATATACATGCG | SEQ ID NO: 4718 | TTCTAAGAGAAGGAAATCTTTCTC | SEQ ID NO: 7084 |
| rs805257 | CCGTGAGAAAAGGAGGAGCCCA | SEQ ID NO: 2353 | CCGTGAGAAAAGGAGGAGCCCG | SEQ ID NO: 4719 | TTCTTGCTTGAGAATGATAAACT | SEQ ID NO: 7085 |
| rs469064 | TCACAAGGACCAGAGCTGTTTAGCA | SEQ ID NO: 2354 | TCACAAGGACCAGAGCTGTTTAGCC | SEQ ID NO: 4720 | CAAGGTGTTGATTAAACTTCAG | SEQ ID NO: 7086 |
| rs1264517 | TCATTGTCACAACCTAGCTGGAACT | SEQ ID NO: 2355 | TCATTGTCACAACCTAGCTGGAACA | SEQ ID NO: 4721 | TACATGAAGTGTCTACAATTGGG | SEQ ID NO: 7087 |
| rs211457 | GACAGCATGAACAGAAGTGAGGACA | SEQ ID NO: 2358 | GACAGCATGAACAGAAGTGAGGACG | SEQ ID NO: 4722 | AACCTAAAAGTGGGAATAACCTC | SEQ ID NO: 7088 |
| rs537160 | CCCAACGATCCTGCTGTTCAACT | SEQ ID NO: 2357 | CCCAACGATCCTGCTGTTCAACC | SEQ ID NO: 4723 | TTGACTACAAAGTGGTCCTCC | SEQ ID NO: 7089 |
| rs2075600 | TGTGATGATAGGGTTACACATCTGCTT | SEQ ID NO: 2358 | TGTGATGATAGGGTTACACATCTGCTC | SEQ ID NO: 4724 | AATTCCTTTCTCTTATGATCTAAC | SEQ ID NO: 7090 |
| rs707926 | CAATGTCATCGATCCCCTGGAT | SEQ ID NO: 2359 | CAATGTCATCGATCCCCTGGAC | SEQ ID NO: 4725 | TCATCTATGGAATCTCCCTG | SEQ ID NO: 7091 |
| rs387608 | TGTATTTTTTTTAACAAGCAGCCCAT | SEQ ID NO: 2360 | TGTATTTTTTTTAACAAGCAGCCCAC | SEQ ID NO: 4726 | TAATTTTGACACAGGCATAATAAC | SEQ ID NO: 7092 |
| rs2071888 | ATGGGACCTTCTGGCTGCCTAC | SEQ ID NO: 2361 | ATGGGACCTTCTGGCTGCCTAG | SEQ ID NO: 4727 | GTTCAACCCTTTCAGGAGG | SEQ ID NO: 7093 |
| rs2073524 | GCGCTGAGTCTGAGGGGAAA | SEQ ID NO: 2362 | GCGCTGAGTCTGAGGGGAAT | SEQ ID NO: 4728 | CTGTGGTACCTTCCTGTCC | SEQ ID NO: 7094 |
| rs805306 | TGGTGGGGCTCTACTCACCAGT | SEQ ID NO: 2363 | TGGTGGGGCTCTACTCACCAGC | SEQ ID NO: 4729 | CTTTGACCTCTCCCATTCTTC | SEQ ID NO: 7095 |
| rs4S6416 | GCCCTGGGAGCAGGGAAACA | SEQ ID NO: 2364 | GCCCTGGGAGCAGGGAAACG | SEQ ID NO: 4730 | CATGGTCAGGTTACTGGGG | SEQ ID NO: 7096 |
| rs652888 | CTGGGGCGAGGAGGCAGA | SEQ ID NO: 2365 | CTGGGGCGAGGAGGCAGG | SEQ ID NO: 4731 | GTCAGCTCAACCCCATGAT | SEQ ID NO: 7097 |
| rs2075798 | TAGGCCTCGTCATCCTCGTCCA | SEQ ID NO: 2366 | TAGGCCTCGTCATCCTCGTCCC | SEQ ID NO: 4732 | CTCAGTCTCCGGAGTGATTG | SEQ ID NO: 7098 |

1. A method comprising:
(a) providing the identity of the nucleotide for each of a set of single nucleotide polymorphisms (SNPs) in the major histocompatability complex (MHC) region in a population of individuals;
(b) providing the HLA type for said individuals; and
(c) identifying a SNP haplotype in said population that correlates with said HLA type, wherein said SNP haplotype comprises said SNPs in said MHC region.

2. The method of claim 1, wherein said HLA type is an HLA serotype.

3. The method of claim 1, wherein linkage disequilibrium is used to correlate said SNP haplotype in said population to said HLA type.

4. The method of claim 1, wherein said set of SNPs comprises at least one SNP selected from the group consisting of the SNPs shown on Table 1.

5. The method of claim 1, wherein said SNP haplotype comprise at least one SNP that occurs outside of the translated region(s) of an HLA gene.

6. The method of claim 1, wherein said MHC region consists essentially of human chromosome 6 between the Ret Finger Protein (RFP) gene and the MLN gene.

7. The method of claim 1, wherein said method further comprises identifying said SNP haplotype as predictive of transplantation compatibility.

8. The method of claim 1, wherein said method further comprises identifying said SNP haplotype as predictive of transfusion compatibility.

9. The method of claim 1, wherein said method further comprises identifying said SNP haplotype as predictive of an immunological or inflammatory condition.

10. The method of claim 9, wherein said immunological or inflammatory condition is selected from the group consisting of inflammatory bowel disease (IBD), ulcerative colitis, Crohn's disease, rheumatoid arthritis, diabetes, type I diabetes mellitus (IDDM), myasthenia gravis, vitiligo, Graves' disease, Hashimoto's disease, Addison's disease, autoimmune gastritis and autoimmune hepatitis, rheumatoid disease, systemic lupus erythematosus (SLE), progressive systemic sclerosis and variants, polymyositis, dermatomyositis, pernicious anemia including some of autoimmune gastritis, primary biliary cirrhosis, autoimmune thrombocytopenia, Sjogren's syndrome, multiple sclerosis (MS), Reiter's syndrome, and psoriasis.

11. The method of claim 1, wherein said haplotype comprises at least 4 SNPs.

12. The method of claim 1, wherein said population of individuals comprises individuals having said HLA type and individuals having a different HLA type.

13. A method for predicting the HLA type of an individual, said method comprising:
(a) providing the identity of the nucleotide for each of a set of SNPs in one or more nucleic acids from said individual that correlate with an HLA type; and
(b) predicting the HLA type of said individual based on a SNP haplotype comprising said set of SNPs.

14. The method of claim 13, wherein said SNPs that correlate with HLA type are identified by a method comprising:
(a) providing the identity of the nucleotide for each of a set of single nucleotide polymorphisms (SNPs) in the major histocompatability complex (MHC) region in a population of individuals;
(b) providing said HLA type for said individuals; and
(c) identifying a SNP haplotype in said population that correlates with said HLA type, wherein said SNP haplotype comprises said SNPs in said MHC region.

15. The method of claim 13, wherein said HLA type is an HLA serotype.

16. The method of claim 13, wherein said set of SNPs comprises at least one SNP selected from the group consisting of the SNPs shown on Table 1.

17. The method of claim 13, wherein said SNP haplotype comprises at least one SNP that occurs outside of the translated region(s) of an HLA gene.

18. The method of claim 13, wherein said haplotype comprises at least 4 SNPs.

19. A method of identifying an SNP haplotype that correlates with an allelic variant of an MHC gene, said method comprising:
(a) providing the identity of the nucleotide for each of a set of single nucleotide polymorphisms (SNPs) in the major histocompatability complex (MHC) region in a population of individuals;
(b) providing the presence or absence of said allelic variant of an MHC gene in said individuals; and
(c) identifying a SNP haplotype in said population that correlates with said allelic variant of an MHC gene, wherein said SNP haplotype comprises said set of SNPs.

20. The method of claim 19, wherein linkage disequilibrium is used to correlate said SNP haplotype in said population to said allelic variant of an MHC gene.

21. The method of claim 19, wherein said set of SNPs comprises at least one SNP selected from the group consisting of the SNPs shown on Table 1.

22. The method of claim 19, wherein said SNP haplotype comprises at least two SNPs that occur outside of the coding region of an HLA gene.

23. The method of claim 19, wherein said MHC region consists essentially of human chromosome 6 between the Ret Finger Protein (RFP) gene and the MLN gene.

24. The method of claim 19, wherein said SNP haplotype comprises at least 4 SNPs.

25. The method of claim 19, wherein said allelic variant of said MHC gene comprises a genetic marker in a translated region of said MHC gene.

26. The method of claim 19, wherein said set of SNPs comprises a SNP that is outside of the translated region(s) of an MHC gene.

27. A method for determining the presence or absence of an allelic variant of an MHC gene in an individual, said method comprising:
(a) providing the identity of the nucleotide for each of a set of SNPs in one or more nucleic acids from said individual that correlate with said allelic variant of an MHC gene; and
(b) determining presence or absence of an allelic variant of an MHC gene in said individual based on a SNP haplotype comprising said set of SNPs.

28. The method of claim 27, wherein said SNPs that correlate with said allelic variant of an MHC gene are identified by a method comprising:
(a) providing the identity of the nucleotide for each of a set of single nucleotide polymorphisms (SNPs) in the major histocompatability complex (MHC) region in a population of individuals;
(b) providing the presence or absence of said allelic variant of an MHC gene in said individuals; and
(c) identifying a SNP haplotype in said population that correlates with said MHC genotype, wherein said SNP haplotype comprises wherein said SNP haplotype comprises said set of SNPs.

29. The method of claim 27, wherein said set of SNPs comprises at least one SNP selected from the group consisting of the SNPs shown on Table 1.

30. The method of claim 27, wherein said SNP haplotype comprises at least 4 SNPs.

31. The method of claim 27, wherein said allelic variant of said MHC gene comprises a genetic marker in a translated region of said MHC gene.

32. The method of claim 27 or 28, wherein said set of SNPs comprises a SNP that is outside of the translated region(s) of said MHC gene

33. A method for identifying a SNP haplotype that correlates with susceptibility to a disease or condition, said method comprising:
(a) providing the identity of the nucleotide for each of a set of single nucleotide polymorphism (SNPs) in the major histocompatability complex (MHC) region in a population of individuals;
(b) providing susceptibility to said disease or condition for said individuals; and
(c) identifying a SNP haplotype in said population that correlates with said susceptibility to said disease, wherein said SNP haplotype comprises said SNPs in the MHC region.

34. The method of claim 33, wherein linkage disequilibrium is used to correlate said SNP haplotype in said population to said susceptibility to said disease.

35. The method of claim 33, wherein said set of SNPs comprises at least one SNP selected from the group consisting of the SNPs shown on Table 1.

36. The method of claim 33, wherein said SNP haplotype comprise at least one SNP that occurs outside of the translated region(s) of an HLA gene.

37. The method of claim 33, wherein said MHC region consists essentially of human chromosome 6 between the Ret Finger Protein (RFP) gene and the MLN gene.

38. The method of claim 33, wherein said disease or condition comprises an immunological or inflammatory condition.

39. The method of claim 38, wherein said immunological or inflammatory condition is selected from the group consisting of inflammatory bowel disease (IBD), ulcerative colitis, Crohn's disease, rheumatoid arthritis, diabetes, type I diabetes mellitus (IDDM), myasthenia gravis, vitiligo, Graves' disease, Hashimoto's disease, Addison's disease, autoimmune gastritis and autoimmune hepatitis, rheumatoid disease, systemic lupus erythematosus (SLE), progressive systemic sclerosis and variants, polymyositis, dermatomyositis, pernicious anemia including some of autoimmune gastritis, primary biliary cirrhosis, autoimmune thrombocytopenia, Sjogren's syndrome, multiple sclerosis (MS), Reiter's syndrome, and psoriasis.

40. The method of claim 33, wherein said haplotype comprises at least 4 SNPs.

41. The method of claim 33, wherein said population of individuals comprises individuals having said disease or condition and individuals that do not have said disease or condition.

42. A method for determining the susceptibility of an individual to a disease or condition, said method comprising:
(a) providing the identity of a set of SNPs in one or more nucleic acids from said individual that correlate with susceptibility to said disease or condition; and
(b) determining said susceptibility of said individual to said disease or condition based on a SNP haplotype comprising said set of SNPs.

43. The method of claim 42, wherein said SNPs that correlate with susceptibility to said disease or condition are identified by a method comprising the steps of:
(a) providing the identity of the nucleotide for each of a set of single nucleotide polymorphism (SNPs) in the major histocompatability complex (MHC) region in a population of individuals;
(b) providing susceptibility to said disease or condition for said individuals; and
(c) identifying a SNP haplotype in said population that correlates with said susceptibility to said disease or condition, wherein said SNP haplotype comprises said SNPs in the MHC region.

44. The method of claim 42, wherein said set of SNPs comprises at least one SNP selected from the group consisting of the SNPs shown on Table 1.

45. The method of claim 42, wherein said SNP haplotype comprise at least one SNP that occurs outside of the translated region(s) of an HLA gene.

46. The method of claim 42, wherein said disease or condition comprises an immunological or inflammatory condition.

47. The method of claim 46, wherein said immunological or inflammatory condition is selected from the group consisting of inflammatory bowel disease (IBD), ulcerative colitis, Crohn's disease, rheumatoid arthritis, diabetes, type I diabetes mellitus (IDDM), myasthenia gravis, vitiligo, Graves' disease, Hashimoto's disease, Addison's disease, autoimmune gastritis and autoimmune hepatitis, rheumatoid disease, systemic lupus erythematosus (SLE), progressive systemic sclerosis and variants, polymyositis, dermatomyositis, pernicious anemia including some of autoimmune gastritis, primary biliary cirrhosis, autoimmune thrombocytopenia, Sjogren's syndrome, multiple sclerosis (MS), Reiter's syndrome, and psoriasis.

48. The method of claim 40, wherein said haplotype comprises at least 4 SNPs.
